(19) United States
(12) Patent Application Publication
(10) Pub. No.: US 2018/0310561 A1
(43) Pub. Date: Nov. 1, 2018

(54) Pyridine Compounds for Combating Phytopathogenic Fungi

(71) Applicant: BASF SE, Ludwigshafen (DE)

(72) Inventors: Wassilios GRAMMENOS, Ludwigshafen (DE); Christian Harald WINTER, Ludwigshafen (DE); Bernd MUELLER, Frankenthal (DE); Antje WOLF, Ludwigshafen (DE); Ana ESCRIBANO CUESTA, Mannheim (DE); Erica CAMBEIS, Sale (GB); Jan Klaas LOHMANN, Lambsheim (DE); Thomas GROTE, Wachenheim (DE); Manuel KRETSCHMER, Washington, DC (US); Nadine RIEDIGER, Schifferstadt (DE); Ian Robert CRAIG, Ludwigshafen (DE); Christine WIEBE, Mannheim (DE); Violeta TERTERYAN-SEISER, Mannheim (DE); Andreas KOCH, Birkenheide (DE); Marcus FEHR, Speyer (DE)

(21) Appl. No.: 15/769,856

(22) PCT Filed: Oct. 5, 2016
(86) PCT No.: PCT/EP2016/073720

§ 371 Date: Apr. 20, 2018

(30) Foreign Application Priority Data
Oct. 23, 2015 (EP) 15191271.4

(51) Int. Cl. A01N 43/42; A01N 43/90; C07D 495/04; C07D 401/14

(52) U.S. Cl. CPC A01N 43/42; C07D 401/14; C07D 495/04; A01N 43/90

(57) ABSTRACT

The present invention relates to Compounds of formula I

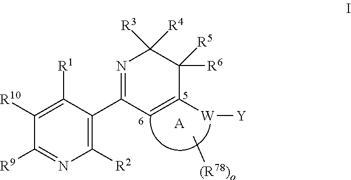

wherein the variables are defined as given in the description and claims. The invention further relates to uses and composition for compounds of formula I.

The present invention relates to pyridine compounds and the N-oxides and the salts thereof for combating phytopathogenic fungi, and to the use and methods for combating phytopathogenic fungi and to seeds coated with at least one such compound. The invention also relates to processes for preparing these compounds, intermediates, processes for preparing such inter-mediates, and to compositions comprising at least one compound I.

In many cases, in particular at low application rates, the fungicidal activity of the known fungicidal compounds is unsatisfactory. Based on this, it was an object of the present invention to provide compounds having improved activity and/or a broader activity spectrum against phytopathogenic harmful fungi.

Surprisingly, this object is achieved by the use of the inventive pyridine compounds of formula I having favorable fungicidal activity against phytopathogenic fungi.

Accordingly, the present invention relates to the compounds of formula I

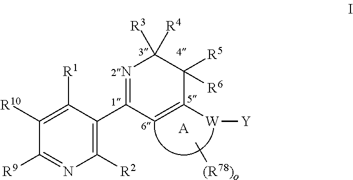

wherein
R¹ is in each case independently selected from H, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein
   R^{x} is C₁-C₄-alkyl, C₁-C₄-haloalkyl, unsubstituted aryl or aryl that is substituted with substituents R^{x1} independently selected from C₁-C₄-alkyl;
   wherein the acyclic moieties of R¹ are unsubstituted or substituted by groups R^{1a} which independently of one another are selected from:
   R^{1a} halogen, OH, CN, C₁-C₆-alkoxy, C₃-C₆-cycloalkyl, C₃-C₆-halocycloalkyl, C₁-C₄-haloalkoxy, C₁-C₆-alkylthio and phenoxy, wherein the phenyl group is unsubstituted or substituted by substituents R^{11a} selected from the group consisting of halogen, OH, C₁-C₄-alkyl, C₁-C₄-haloalkyl, C₁-C₄-alkoxy and C₁-C₄-haloalkoxy;
   wherein the cycloalkyl, heteroaryl and aryl moieties of R¹ are unsubstituted or substituted by groups R^{1b} which independently of one another are selected from:
   R^{1b} halogen, OH, CN, C₁-C₄-alkyl, C₁-C₄-alkoxy, C₁-C₄-haloalkyl, C₃-C₆-cycloalkyl, C₃-C₆-halocycloalkyl, C₁-C₄-haloalkoxy and C₁-C₆-alkylthio;
R² is in each case independently selected from the substituents as defined for R¹, wherein the possible substituents for R² are R^{2a} and R^{2b}, respectively, which correspond to R^{1a} and R^{1b}, respectively;
R³, R⁴ are independently selected from halogen, OH, CN, NO₂, SH, C₁-C₆-alkylthio, C₁-C₆-haloalkylthio, NH₂, NH(C₁-C₄-alkyl), N(C₁-C₄-alkyl)₂, NH—SO₂—R^{x}, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₃-C₆-cycloalkyl, C₃-C₆-halocycloalkyl, C₁-C₆-haloalkoxy, CH(═O), C(═O)C₁-C₆-alkyl, C(═O)OC₁-C₆-alkyl, C(═O)NHC₁-C₆-alkyl, C(═O)N(C₁-C₆-alkyl)₂, CR^{x}═NR^{x}, saturated or partially unsaturated three-, four-, five-, six-, seven-, eight-, nine-, or ten-membered carbocycle or heterocycle, wherein in each case one or two CH₂ groups of the carbo- and heterocycle may be replaced by a group independently se-lected from C(═O) and C(═S), five- or six-membered heteroaryl and aryl; wherein the heterocycle and the heteroaryl contain independently one, two, three or four heteroatoms selected from N, O and S; or
R³, R⁴ together with the carbon atom to which they are bound form saturated, partially unsaturated three-, four-, five-, six-, seven-, eight-, nine-, or ten-membered carbo- or heterocycle, wherein the heterocycle contains one, two, three or four heteroatoms selected from N, O and S, wherein the heteroatom N may carry one substituent selected from C₁-C₄-alkyl, C₁-C₄-haloalkyl and SO₂Ph, wherein Ph is unsubstituted phenyl or phenyl that is substituted by one, two or three substituents selected from C₁-C₄-alkyl, halogen, C₁-C₄-haloalkyl, C₁-C₄-alkoxy and C₁-C₄-haloalkoxy, and CN; and wherein the heteroatom S may be in the form of its oxide SO or SO₂, and wherein the carbo- or heterocycle is unsubstituted or carries one, two, three or four substituents R³⁴ independently selected from halogen, OH, CN, NO₂, SH, NH₂, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₄-alkoxy-C₁-C₄-alkyl, phenyl and phenoxy, wherein the phenyl groups are unsubstituted or carry one, two, three, four or five substituents R^{34a} selected from the group consisting of halogen, OH, C₁-C₄-alkyl, C₁-C₄-haloalkyl, C₁-C₄-alkoxy, C₁-C₄-haloalkoxy and CN; and wherein in each case one or two CH₂ groups of the carbo- or heterocycle may be replaced by a group independently selected from C(═O) and C(═S); and
   wherein the acyclic moieties of R³ and R⁴ are independently unsubstituted or substituted by groups R^{1a} or R^{4a}, respectively, which independently of one another are selected from:
   R^{3a}, R^{4a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, C₁-C₆-alkoxy, C₃-C₆-cycloalkyl, C₃-C₆-halocycloalkyl, C₁C₄-haloalkoxy, C₁-C₆-alkylthio, CH(═O), C(═O)C₁-C₆-alkyl, C(═O)OC₁-C₆-alkyl, C(═O)NHC₁-C₆-alkyl, C(═O)N(C₁-C₆-alkyl)₂, CR^{x}═NR^{x}, a saturated or partially unsaturated three-, four-, five-, six-, seven-, eight-, nine-, or ten-membered carbo- or heterocycle, wherein in each case one or two CH₂ groups of the carbo- and heterocycle may be replaced by a group independently selected from C(═O) and C(═S), an aryl and phenoxy, wherein the aryl and phenyl groups are independently unsubstituted or substituted with substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkoxy alkyl)₂, NH—SO₂—R^{x}, C₁-C₆-alkylthio, C₁-C₄-alkyl, C₁-C₄-haloalkyl, C₁-C₄-alkoxy and C₁-C₄-haloalkoxy;
   wherein the carbocyclic, heterocyclic, heteroaryl and aryl moieties of R³ and R⁴ are independently unsubstituted or substituted with identical or different groups R^{3b} or R^{4b}, respectively, which independently of one another are selected from:
R^{3b}, R^{4b} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-haloalkyl, C₃-C₆-cycloalkyl, C₃-C₆-halocycloalkyl, C₁-C₄-haloalkoxy, C₁-C₆-alkylthio, C₁-C₆-haloalkylthio, C₁-C₄-alkoxy-C₁-C₄-haloalkyl, phenyl and phenoxy, wherein the phenyl groups are unsubstituted or carry one, two, three, four or five substituents selected from the group consisting of halogen, OH, C₁-C₄-alkyl, C₁-C₄-haloalkyl, C₁-C₄-alkoxy and C₁-C₄-haloalkoxy;
   and wherein R^{x} is as defined above; and
R⁵ is H;
R⁶ is H;
ring A-W—Y is selected from below group:

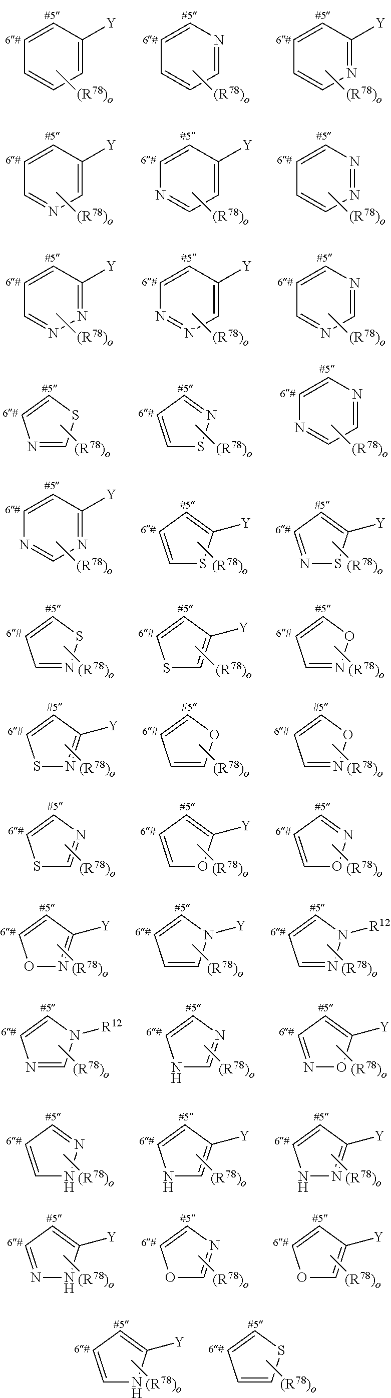

wherein, the positions of the rings marked with “#” represents the connection points (carbon atoms 5″ and 6″ in formula I) with the remaining skeleton of the compounds of formula I.; wherein
o is 0, 1, 2 or 3; and
R⁷⁸ are independently selected from halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, CH(═O), C(═O)C₁-C₆-alkyl, C(═O)NH(C₁-C₆-alkyl), CR′═NOR″, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₂-C₆-alkenyloxy, C₂-C₆-alkynyloxy, C₃-C₆-cycloalkyl, C₃-C₆-cycloalkenyl, three-, four-, five- or six-membered saturated or partially unsaturated heterocycle, five- or six-membered heteroaryl and phenyl; wherein the heterocycle or heteroaryl contains one, two or three heteroatoms selected from N, O and S; wherein R′ and R″ are independently selected from H, C₁-C₄-alkyl, C₂-C₆-alkenyl, C₂-C₆-alkynyl, C₃-C₆-cycloalkyl, saturated or partially unsaturated three-, four-, five-, six-, seven-, eight-, nine-, or ten-membered heterocycle, five- or six-membered heteroaryl or aryl; wherein the heterocycle or heteroaryl contains one, two or three heteroatoms selected from N, O and S, and wherein R′ and R″ are independently unsubstituted or substituted by R″' which is independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₃-C₆-cycloalkyl, C₃-C₆-halogencycloalkyl and phenyl; and wherein R^{x} is defined above; and
   wherein the acyclic moieties of R⁷⁸ are unsubstituted or substituted by R^{78a} which independently of one another are selected from:
   R^{78a} halogen, OH, CN, C₁-C₆-alkoxy, C₃-C₆-cycloalkyl, C₃-C₆-cycloalkenyl, C₃-C₆-halocycloalkyl, C₃-C₆-halocycloalkenyl, C₁-C₄-haloalkoxy, C₁-C₆-alkylthio, five- or six-membered heteroaryl, phenyl and phenoxy, wherein the heteroaryl, phenyl and phenoxy group is unsubstituted or substituted by ^{R78aa} selected from the group consisting of halogen, OH, C₁-C₄-alkyl, C₁-C₄-haloalkyl, C₁-C₄-alkoxy and C₁C₄-haloalkoxy;
   wherein the alicyclic, phenyl, heterocyclic and heteroaryl moieties of R⁷⁸ are unsubstituted or substituted by R^{78b} which independently of one another are selected from:
   R^{78b} halogen, OH, CN, C₁-C₄-alkyl, C₁-C₄-alkoxy, C₁-C₄-haloalkyl, C₃-C₆-cycloalkyl, C₃-C₆-halocycloalkyl, C₁-C₄-haloalkoxy, and C₁-C₆-alkylthio;
Y is independently selected from F, Cl, Br, I, cyano, nitro, C₁-C₆-alkyl, C₁-C₆-haloalkyl, C₂-C₆-alkenyl, C₁-C₆-haloalkoxy, C₂-C₆-alkynyl, C₃-C₆-cycloalkyl, C₃-C₆-cycloalkenyl, —O(Y¹), —S(O)_{z}(Y²), —N(Y³)(Y⁴), C₁-C₆-haloalkyl, C₂-C₆-haloalkenyl, C₂-C₆-haloalkynyl, —CO(Y⁵), —C(Y⁶)═NO(Y⁷); wherein the acyclic moieties of Y are unsubstituted or substituted by R^{78a} and the alicyclic, phenyl and heteroaryl moieties of Y are unsubstituted or substituted by R^{78b};
   z is 0, 1 or 2;
   Y¹ is selected from H, C₁-C₆-alkyl, C₂-C₆-alkenyl, C₂-C₆-alkynyl, cycloalkyl, cycloalkenyl, C₁-C₆-haloalkyl, C₂-C₆-haloalkenyl, C₂-C₆-haloalkynyl, phenyl and heteroaryl;
   Y² is selected from H, C₁-C₆-alkyl, C₂-C₆-alkenyl, C₂-C₆-alkynyl, cycloalkyl, cycloalkenyl, C₁-C₆-haloalkyl, C₂-C₆-haloalkenyl, C₂-C₆-haloalkynyl, phenyl and heteroaryl;
   Y³, Y⁴ are selected from H, C₁-C₆-alkyl, C₂-C₆-alkenyl, C₂-C₆-alkynyl, cycloalkyl, cycloalkenyl, C₁-C₆-haloalkyl, C₂-C₆-haloalkenyl, C₂-C₆-haloalkynyl, phenyl, heteroaryl and CO(Y³¹);

Y³¹ is selected from H, C₁-C₆-alkyl, C₂-C₆-alkenyl, C₂-C₆-alkynyl, cycloalkyl, cycloalkenyl, C₁-C₆-haloalkyl, C₂-C₆-haloalkenyl, C₂-C₆-haloalkynyl, phenyl, heteroaryl, -0(Y³¹¹) and —N(Y³¹²)(Y³¹³);
Y³¹¹ is selected from C₁-C₆-alkyl, C₂-C₆-alkenyl, C₂-C₆-alkynyl, cycloalkyl, cycloalkenyl, C₁-C₆-haloalkyl, C₂-C₆-haloalkenyl, C₂-C₆-haloalkynyl, phenyl and heteroaryl;
Y³¹², Y³¹³ are selected from H, C₁-C₆-alkyl, C₂-C₆-alkenyl, C₂-C₆-alkynyl, cycloalkyl, cycloalkenyl, C₁-C₆-haloalkyl, C₂-C₆-haloalkenyl, C₂-C₆-haloalkynyl, phenyl and heteroaryl;
Y⁵ is selected from H, C₁-C₆-alkyl, C₂-C₆-alkenyl, C₂-C₆-alkynyl, cycloalkyl, cycloalkenyl, C₁-C₆-haloalkyl, C₂-C₆-haloalkenyl, C₂-C₆-haloalkynyl, phenyl and heteroaryl;
Y⁶ is selected from H, C₁-C₆-alkyl, C₂-C₆-alkenyl, C₂-C₆-alkynyl, cycloalkyl, cycloalkenyl, C₁-C₆-haloalkyl, C₂-C₆-haloalkenyl, C₂-C₆-haloalkynyl, phenyl and heteroaryl;
Y⁷ is selected from H, C₁-C₆-alkyl, C₂-C₆-alkenyl, C₂-C_{6—}alkynyl, cycloalkyl, cycloalkenyl, C₁-C₆-haloalkyl, C₂-C₆-haloalkenyl, C₂-C₆-haloalkynyl, phenyl and heteroaryl;
R⁹, R¹⁰ are independently selected from H, halogen, CN, NO₂, N(R⁹¹)(R⁹²), S(R⁹³), S(O)_{z94}(R⁹⁴), O(R⁹⁵), C₁-C₆-alkyl, C₂-C₆-alkenyl, C₂-C₆-alkynyl, cycloalkyl, CO—(R⁹⁶) and CS—(R⁹⁶);
   R⁹¹, R⁹² are independently selected from H, alkyl, alkenyl, alkynyl, cycloalkyl, CO—R(⁹¹¹), and S(O)_{z91}R⁹¹²;
   R⁹¹¹ is H or R⁹¹²;
   R⁹¹² is independently selected from alkyl, alkenyl, alkynyl, cycloalkyl, O—R⁹¹¹¹, and N(R⁹¹¹²)(R⁹¹¹³);
   R⁹¹¹¹ is alkyl, alkenyl, alkynyl alkenyl, or cycloalkyl;
   R⁹¹¹², R⁹¹¹³ are independently selected from H, alkyl, alkenyl, alkynyl, and cycloalkyl;
   z⁹¹ is 1 or 2;
   R⁹³ is H, alkyl, alkenyl, alkynyl cycloalkyl;
   R⁹⁴ is independently selected from alkyl, alkenyl, alkynyl, cycloalkyl, O—R⁹⁴¹, N(R⁹⁴²)(R⁹⁴³);
   R⁹⁴¹ is independently selected from alkyl, alkenyl, alkynyl, cycloalkyl;
   R⁹⁴², R⁹⁴³ are independently selected from H or R⁹⁴¹;
   z⁹⁴ is 1 or 2;
   R⁹⁵ is independently selected from H, alkyl, alkenyl, alkynyl, cycloalkyl, carbonyl-R⁹⁵¹, S(O)_{z95}R⁹⁵²;
   R⁹⁵¹ is H or R⁹⁵²;
   R⁹⁵² is independently selected from alkyl, alkenyl, alkynyl, cycloalkyl, O—R⁹⁵²¹, N(R⁹⁵²²)(R⁹⁵²³);
   R⁹⁵²¹ is independently selected from alkyl, alkenyl, alkynyl, cycloalkyl;
   R9522, R⁹⁵²³ is independently selected from H and R⁹⁵²¹;
   z⁹⁵ is 1 or 2;
   R⁹⁶ is independently selected from H, alkyl, alkenyl, alkynyl, cycloalkyl, O—R⁹⁶¹, N(R⁹⁶²)(R⁹⁶³);
   R⁹⁶¹ is independently selected from H, alkyl, alkenyl, alkynyl, cycloalkyl;
   R^{962,} R⁹⁶³ are independently selected from H, alkyl, alkenyl, alkynyl, cycloalkyl;
   wherein the acyclic moieties of R⁹, R¹⁰ are unsubstituted or substituted by identical or different groups R⁹a, wherein:
   R^{9a} independently of one another are selected from: halogen, OH, CN, C₁-C₆-alkoxy, alkenyloxy, alkynyloxy, C₃-C₆-cycloalkyl, C₃-C₆-halogencycloalkyl, C₁-C₄-haloalkoxy, C₁-C₆-alkylthio, C(═O) and C(═S); or

R⁹ & R¹⁹ together with the carbon atoms to which they are bound form a five- , six-, or seven-membered carbo- and heterocyclic or heteroaromatic ring; wherein the heterocyclic or heteroaromatic ring contains 1, 2, 3 or 4 heteroatoms selected from N, O and S, wherein N may carry one substituent R^{N} selected from C₁-C₄-alkyl, C₁-C₄-haloalkyl and SO₂Ph, wherein Ph is unsubstituted or substituted by substituents selected from C₁-C₄-alkyl, and wherein S may be in the form of its oxide SO or SO₂; and wherein in each case one or two CH₂ groups of the carbo- or heterocycle may be replaced by a group independently selected from C(═O) and C(═S); and wherein the carbo- and heterocyclic or heteroaromatic ring is substituent by (R¹¹)ₘ, wherein m is 0, 1, 2, 3 or 4;
R¹¹ is in each case independently selected from halogen, OH, CN, NO2, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
   R^{x} is C₁-C₄-alkyl, C₁-C₄-haloalkyl, aryl which is unsubstituted or substituted by substituents R^{x1} independently selected from C₁-C₄-alkyl;
      wherein the acyclic moieties of R¹¹ are unsubstituted or substituted with identical or different groups R¹¹a which independently of one another are selected from:
   R^{11a} halogen, OH, CN, C₁-C₆-alkoxy, C₃-C₆-cycloalkyl, C₃-C₆-halogencycloalkyl, C₁-C₄-haloalkoxy, C₁-C₆-alkylthio and phenoxy, wherein the phenyl group is un-substituted or unsubstituted or substituted with R^{11a} selected from the group consisting of halogen, OH, C₁-C₄-alkyl, C₁-C₄-haloalkyl, C₁-C₄-alkoxy and C₁-C₄-haloalkoxy;
      wherein the cycloalkyl, heteroaryl and aryl moieties of R¹¹ unsubstituted or substituted with identical or different groups R^{11b} which independently of one another are selected from:
      R^{11b} halogen, OH, CN, C₁-C₄-alkyl, C₁-C₄-alkoxy, C₁-C₄-haloalkyl, C₃-C₆-cycloalkyl, C₃-C₆-halogencycloalkyl, C₁C₄-haloalkoxy, and C₁-C₆-alkylthio;
   wherein if the A is phenyl
   Y is not F.
   and the N-oxides and the agriculturally acceptable salts thereof.

The numbering of the ring members in the compounds of the present invention is as given in formula I above:

Compounds of formula I can be accessed e.g. starting from alcohols of type II with nitriles of type III in the presence of an acid in an organic solvent (see for example US 2008/0275242 or WO2005/070917). Preferably, sulfuric acid or a sulfonic acid, in particular triflic acid, are used as acid. Most suitable solvents are hydrocarbons, preferably benzene or dichloromethane. In the following schemes, the optionally substituted phenyl or heteroaryl formed by W together with the partially substituted pyridine ring to which it is attached is sketched by a semicircle named “A”.

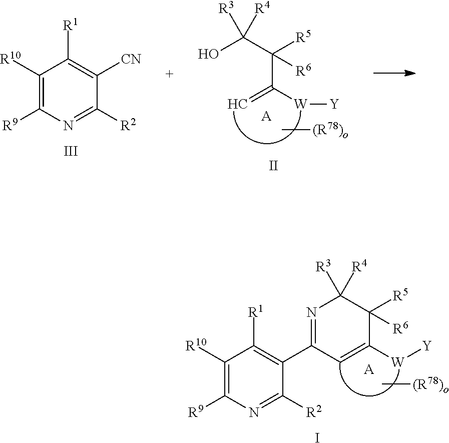

Depending on the nature of the starting materials, the reaction is performed at a temperature from −40° C. to 200° C., in particular from −10° C. to 120° C., more specifically from 0° C. to 100° C., even more specifically from room or ambient temperature (about 23° C.) to 80° C. Nitriles of type III are either commercially available or can be prepared by a skilled person from the corresponding halides following literature precedures (see, for example Journal of Organic Chemistry, 76(2), 665-668; 2011; Angewandte Chemie, International Edition, 52(38), 10035-10039; 2013; WO2004/013094).

Alcohols of type II can be prepared as described below. A skilled person will realize that compounds of type IIIb can be reacted with organometallic reagents, preferably alkyl Grignard or alkyl-Lithium reagents, in ethereal solvents, preferably THF at low temperatures and under inert conditions to furnish compounds of type II.

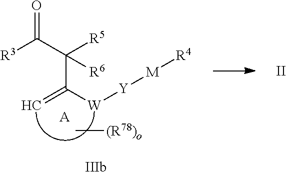

Alternatively, alcohols of type II can be prepared from epoxydes IIIa and compounds VI (see below):

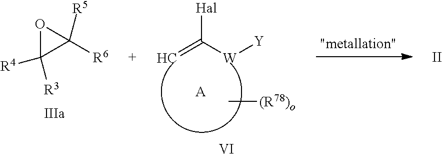

The metallation reaction may preferably be carried out using Lithium-organic compounds, such as for example n-butyl lithium, sec-butyl lithium or tert-butyl lithium to result in an exchange of halogen by lithium. Also suitable is the reaction with magnesium resulting in the formation of the respective Grignard reagents. A further possibility is the use of other Grignard reagents such as isopropyl-magnesium-bromide instead of Mg.

A typical preparation of compounds of type IIIb can be achieved by reacting compounds of type IV with organometallic reagents, preferably alkyl Grignard or alkyl-Lithium reagents, in ethereal solvents, preferably THF at low temperatures and under inert conditions to furnish compounds of type III as previously reported (see for example WO2012051036; WO2011042918).

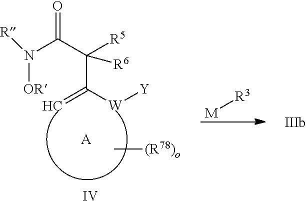

Compounds of type IV can be accessed by reacting a carbonyl compound of type V, preferably a carboxylic acid(X═OH) or an acid chloride (X═Cl), with NH(OR′)R″, wherein R′ and R″ are selected from (C₁-C₄)-alkyl, most preferably being methyl, in an organic solvent, preferably THF or dichloromethane. Typically the reaction is performed in a range between 0° C. and ambient temperature in the presence of an organic base, preferably N(C₂H₅)₃ or pyridine (see e.g. US 20130324506; Tetrahedron: Asymmetry, 17(4), 508-511; 2006). If X═OH, the addition of an activating reagent, preferably a carbodiimide, may be preferred (see for example ChemMedChem, 7(12), 2101-2112; 2012; 2011038204; Journal of Organic Chemistry, 76(1), 164-169; 2011).

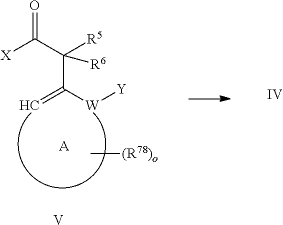

If required, compounds of type V can be prepared from the corresponding aryl halides of type VI (Hal is halogen, preferably Br or I). As described (Tetrahedron, 68(9), 2113-2120; 2012; Chemical Communications (Cambridge, United Kingdom), 49(60), 6767-6769; 2013), aryl halides will react with compounds of type VII in the presence of a transition metal catalyst, preferably a copper(I) salt, in an organic solvent, preferably DMF or DMSO, at elevated temperatures. Typically a base, preferably potassium phosphate, is added.

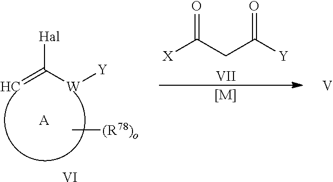

If appropriate, compounds of type II can be prepared as follows. A known or commercially available compound of type VIII can be reacted with an organometallic reagent of type IX, preferably a Grignard or an organolithium reagent, readily prepared by a skilled person. Preferably, the reaction is performed in a temperature range from −78° C. to room temperature under inert conditions in an ethereal solvent.

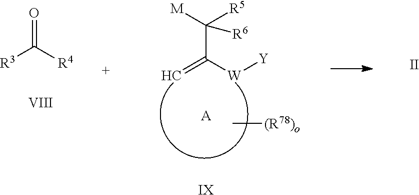

Alternatively compounds I in which R⁵ stands for hydrogen can be accessed by reacting a nitrile III with an olefin II^{#} under acidic conditions as described elsewhere (U.S. Pat. No. 7,632,783, B2, page 60, method A).

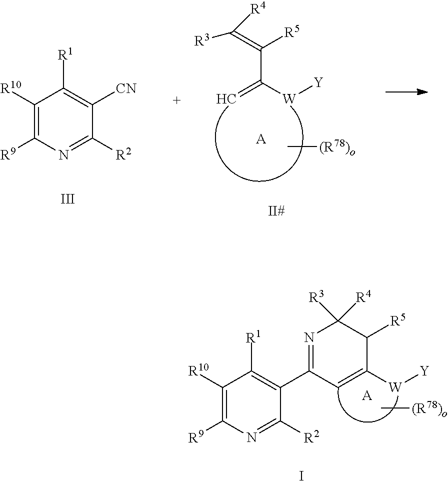

Alternatively compounds I can be prepared via intramolecular reaction of amide X when A is an electron-rich carbon- or heterocycle. The intramolecular cyclization will take place in the presence of a dehydrating agent in an organic solvent (WO 2008143263, Synthetic Communications 2007, 37, 1331-1338.). Preferably, phosphoryl chloride (POCl₃), POCl₃/P₂O₅, H₃PO₄/P₂O₅, SnCl₄ or BF₃ are used as dehydrating agent. Most suitable solvents are hydrocarbons, preferably benzene, toluene or acetonitrile.

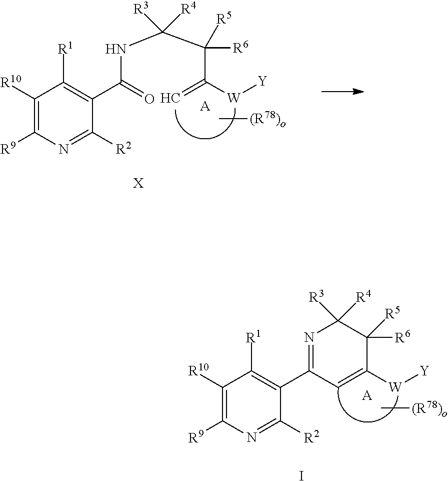

Depending on the nature of starting materials, the reaction is performed at temperature from −40° C. to 200° C., in particular from −10° C. to 120° C., more specifically from 0° C. to 100° C., even more specifically from room temperature to 100° C.

Amides of type X can accessed by reacting a carbonyl of type XI, preferably a carboxylic acid (X═OH) or an acid chloride (X═Cl), with an amines of type XII in an organic solvent, preferably THF or dichloromethane. Typically the reaction is performed in a range between 0° C. and room temperature in the presence of an organic base, preferably N(C₂H₅)₃ or pyridine (see e.g. WO 8303968). If X═OH, the addition of an activating agent, preferably a carbodiimide or acid chloride, may be preferred (see e.g Bioorganic & Medicinal Chemistry, 2010, 18, 3088-3115).

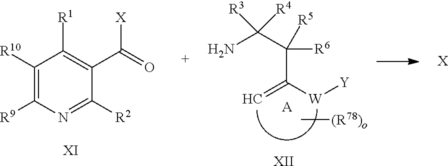

If required, compounds of type XII can be synthesized from the correspond nitriles. As de-scribed Synlett. 2007, 4 652-654 or Tetrahedron 2012, 68, 2696-2703, nitriles will react with organometallic agents, preferably Grignard or Lithium reagent, in ethereal solvents, preferably THF at low temperature and under inert conditions to furnish compounds of type XII. The synthesis of compounds of type XII can take place in two steps or one pot.

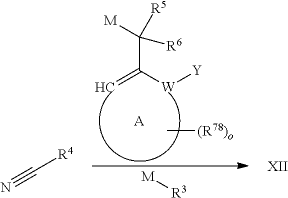

The N-oxides may be prepared from the inventive compounds according to conventional oxidation methods, e.g. by treating compounds I with an organic peracid such as metachloroperbenzoic acid (cf. WO 03/64572 or J. Med. Chem. 38(11), 1892-903, 1995); or with inorganic oxidizing agents such as hydrogen peroxide (cf. J. Heterocyc. Chem. 18(7), 1305-8, 1981) or oxone (cf. J. Am. Chem. Soc. 123(25), 5962-5973, 2001). The oxidation may lead to pure mono-N-oxides or to a mixture of different N-oxides, which can be separated by conventional methods such as chromatography.

In the following, the intermediate compounds are further described. A skilled person will readily understand that the preferences for the substituents, also in particular the ones given in the tables below for the respective substituents, given herein in connection with compounds I apply for the intermediates accordingly. Thereby, the substituents in each case have independently of each other or more preferably in combination the meanings as defined herein.

The intermediate compounds of formula X are novel. Consequently, one aspect of the present invention relates to compounds of formula X:

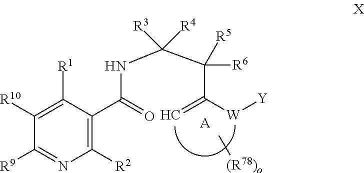

The compounds of formula X have fungicidal activity and the details below referring to the compounds I also apply to compounds X.

Particular embodiments of the compounds IIA are the following compounds

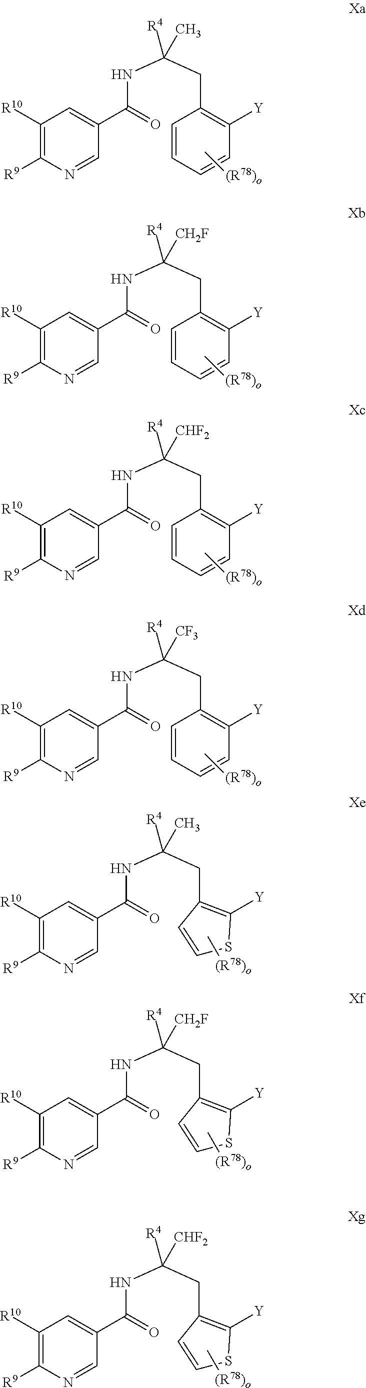

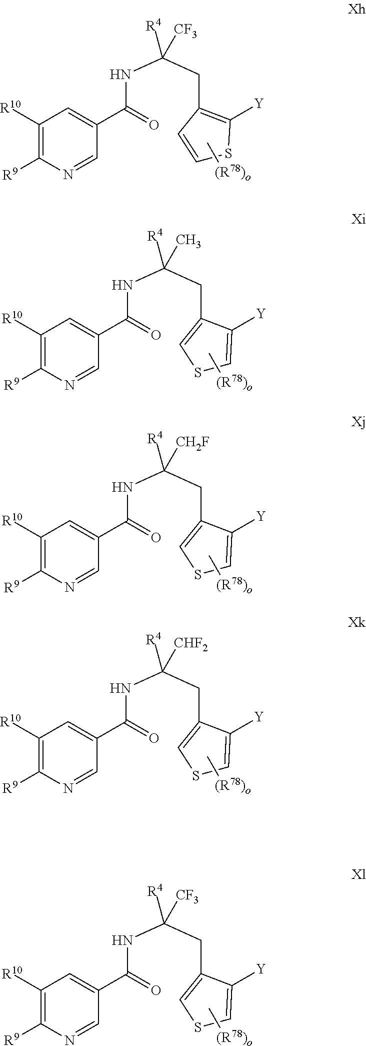

If the synthesis yields mixtures of isomers, a separation is generally not necessarily required since in some cases the individual isomers can be interconverted during work-up for use or during application (e.g. under the action of light, acids or bases). Such conversions may also take place after use, e.g. in the treatment of plants in the treated plant, or in the harmful fungus to be controlled.

In the following, the intermediate compounds are further described. A skilled person will readily understand that the preferences for the substituents, also in particular the ones given in the tables below for the respective substituents, given herein in connection with compounds I apply for the intermediates accordingly. Thereby, the substituents in each case have independently of each other or more preferably in combination the meanings as defined herein.

In the definitions of the variables given above, collective terms are used which are generally representative for the substituents in question. The term “Cₙ-Cₘ” indicates the number of carbon atoms possible in each case in the substituent or substituent moiety in question.

The term “halogen” refers to fluorine, chlorine, bromine and iodine.

The term “C₁-C₆-alkyl” refers to a straight-chained or branched saturated hydrocarbon group having 1 to 6 carbon atoms, e.g. methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Likewise, the term “C₂-C₄-alkyl” refers to a straight-chained or branched alkyl group having 2 to 4 carbon atoms, such as ethyl, propyl (n-propyl), 1-methylethyl (iso-propoyl), butyl, 1-methylpropyl (sec.-butyl), 2-methylpropyl (iso-butyl), 1,1-dimethylethyl (tert.-butyl). The term “C₁-C₆-haloalkyl” refers to an alkyl group having 1 or 6 carbon atoms as defined above, wherein some or all of the hydrogen atoms in these groups may be replaced by halogen atoms as mentioned above. Examples are “C₁-C₂-haloalkyl” groups such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl or pentafluoroethyl. The term “C₁-C₆-hydroxyalkyl” refers to an alkyl group having 1 or 6 carbon atoms as defined above, wherein some or all of the hydrogen atoms in these groups may be replaced by OH groups.

The term “C₁-C₄-alkoxy-C₁-C₄-alkyl” refers to alkyl having 1 to 4 carbon atoms (as defined above), wherein one hydrogen atom of the alkyl radical is replaced by a C₁-C₄-alkoxy group (as defined above). Likewise, the term “C₁-C₆-alkoxy-C₁-C₄-alkyl” refers to alkyl having 1 to 4 carbon atoms (as defined above), wherein one hydrogen atom of the alkyl radical is replaced by a C₁-C₆-alkoxy group (as defined above).

The term “C₂-C₆-alkenyl” refers to a straight-chain or branched unsaturated hydrocarbon radical having 2 to 6 carbon atoms and a double bond in any position. Examples are “C₂-C₄-alkenyl” groups, such as ethenyl, 1-propenyl, 2-propenyl (allyl), 1-methylethenyl, 1-butenyl, 2-butenyl, 3-butenyl, 1-methyl-1-propenyl, 2-methyl-1-propenyl, 1-methyl-2-propenyl, 2-methyl-2-propenyl. The term “C₂-C₆-alkynyl” refers to a straight-chain or branched unsaturated hydrocarbon radical having 2 to 6 carbon atoms and containing at least one triple bond. Examples are “C₂-C₄-alkynyl” groups, such as ethynyl, prop-1-ynyl, prop-2-ynyl (propargyl), but-1-ynyl, but-2-ynyl, but-3-ynyl, 1-methyl-prop-2-ynyl.

The term “C₁-C₆-alkoxy” refers to a straight-chain or branched alkyl group having 1 to 6 carbon atoms which is bonded via an oxygen, at any position in the alkyl group. Examples are “C₁-C₄-alkoxy” groups, such as methoxy, ethoxy, n-propoxy, 1-methylethoxy, butoxy, 1-methyl-prop-oxy, 2-methylpropoxy or 1,1-di-methylethoxy.

The term “C₁-C₆-haloalkoxy” refers to a C₁-C₆-alkoxy radical as defined above, wherein some or all of the hydrogen atoms in these groups may be replaced by halogen atoms as mentioned above. Examples are “C₁-C₄-haloalkoxy” groups, such as OCH₂F, OCHF₂, OCF₃, OCH₂Cl, OCHCl₂, OCCl₃, chlorofluoromethoxy, dichlorofluoromethoxy, chlorodifluoromethoxy, 2-fluoro-ethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoro-propoxy, 2 chloropropoxy, 3-chloropropoxy, 2,3-dichloropropoxy, 2-bromo'propoxy, 3 bromopropoxy, 3,3,3-trifluoropropoxy, 3,3,3-trichloropropoxy, OCH₂—C₂F₅, OCF₂-C₂F₅, 1-fluoromethyl-2-fluoroethoxy, 1-chloromethyl-2-chloroethoxy, 1-bromomethyl-2-bromo-ethoxy, 4-fluorobutoxy, 4-chlorobutoxy, 4-bromobutoxy or nonafluorobutoxy.

The term “C₂-C₆-alkenyloxy” refers to a straight-chain or branched alkenyl group having 2 to 6 carbon atoms which is bonded via an oxygen, at any position in the alkenyl group. Examples are “C₂-C₄-alkenyloxy” groups.

The term “C₂-C₆-alkynyloxy” refers to a straight-chain or branched alkynyl group having 2 to 6 carbon atoms which is bonded via an oxygen, at any position in the alkynyl group. Examples are “C₂-C₄-alkynyloxy” groups.

The term “C₃-C₆-cycloalkyl” refers to monocyclic saturated hydrocarbon radicals having 3 to 6 carbon ring members, such as cyclopropyl, cyclobutyl, cyclopentyl, cyclohexyl. Accordingly, a saturated three-, four-, five-, six-, seven-, eight-, nine or ten-membered carbocyclyl or carbocycle is a “C₃-C₁₀-cycloalkyl”.

The term “C₃-C₆-cycloalkenyl” refers to a monocyclic partially unsaturated 3-, 4- 5- or 6-membered carbocycle having 3 to 6 carbon ring members and at least one double bond, such as cyclopentenyl, cyclopentadienyl, cyclohexadienyl. Accordingly, a partially unsaturated three-, four-, five-, six-, seven-, eight-, nine or ten-membered carbocyclyl or carbocycle is a “C₃-C₁₀-cycloalkenyl”.

The term “C₃-C₆-cycloalkyl-C₁-C₄-alkyl” refers to alkyl having 1 to 4 carbon atoms (as defined above), wherein one hydrogen atom of the alkyl radical is replaced by a cycloalkyl radical having 3 to 8 carbon atoms (as defined above).

The term “C₁-C₆-alkylthio” as used herein refers to straight-chain or branched alkyl groups having 1 to 6 carbon atoms (as defined above) bonded via a sulfur atom. Accordingly, the term “C₁-C₆-haloalkylthio” as used herein refers to straight-chain or branched haloalkyl group having 1 to 6 carbon atoms (as defined above) bonded through a sulfur atom, at any position in the haloalkyl group.

The term “C(═O)—C₁-C₆-alkyl” refers to a radical which is attached through the carbon atom of the group C(═O) as indicated by the number valence of the carbon atom. The number of valence of carbon is 4, that of nitrogen is 3. Likewise the following terms are to be construed: NH(C₁-C₄-alkyl), N(C₁-C₄-alkyl)₂, NH(C₃-C₆-cycloalkyl), N(C₃-C₆-cycloalkyl)₂, C(═O)—NH(C₁-C₆-alkyl), C(═O)-N(C₁-C₆-alkyl)₂.

The term “saturated or partially unsaturated three-, four-, five-, six-, seven-, eight-, nine or ten-membered heterocyclyl or heterocycle, wherein the heterocyclyl or heterocycle contains 1, 2, 3 or 4 heteroatoms selected from N, O and S” is to be understood as meaning both saturated and partially unsaturated heterocycles, wherein the ring member atoms of the heterocycle include besides carbon atoms 1, 2, 3 or 4 heteroatoms independently selected from the group of O, N and S. For example:

a 3- or 4-membered saturated heterocycle which contains 1 or 2 heteroatoms from the group consisting of O, N and S as ring members such as oxirane, aziridine, thiirane, oxetane, azetidine, thiethane, [1,2]dioxetane, [1,2]dithietane, [1,2]diazetidine; and a 5- or 6-membered saturated or partially unsaturated heterocycle which contains 1, 2 or 3 heteroatoms from the group consisting of 0, N and S as ring members such as 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydro-pyranyl, 2-tetrahydrothienyl, 3-hexahydropyridazinyl, 4-hexahydropyridazinyl, 2-hexahydro-pyrimidinyl, 4-hexahydropyrimidinyl, 5-hexahydropyrimidinyl, 2-piperazinyl, 1,3,5-hexahydro-triazin-2-yl and 1,2,4-hexahydrotriazin-3-yl and also the corresponding -ylidene radicals; and a 7-membered saturated or partially unsaturated heterocycle such as tetra- and hexahydroaze-pinyl, such as 2,3,4,5-tetrahydro[1H]azepin-1-,-2-,-3-,-4-,-5-,-6- or -7-yl, 3,4,5,6-tetra-hydro[2H]azepin-2-,-3-,-4-,-5-,-6- or -7-yl, 2,3,4,7-tetrahydro[1H]azepin-1-,-2-,-3-,-4-,-5-,-6- or -7-yl, 2,3,6,7-tetrahydro[1H]azepin-1-,-2-,-3-,-4-,-5-,-6- or -7-yl, hexahydroazepin-1-,-2-,-3- or -4-yl, tetra- and hexahydrooxepinyl such as 2,3,4,5-tetrahydro[1H]oxepin-2-,-3-,-4-,-5-,-6- or -7-yl, 2,3,4,7-tetrahydro[1H]oxepin-2-,-3-,-4-,-5-,-6- or -7-yl, 2,3,6,7-tetrahydro[1H]oxepin-2-, -3-,-4-,-5-, -6- or -7-yl, hexahydroazepin-1-,-2-,-3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding -ylidene radicals.

The term “substituted” refers to substitued with 1, 2, 3 or up to the maximum possible number of substituents.

term “5-or 6-membered heteroaryl” or “5-or 6-membered heteroaromatic” refers to aromatic ring systems incuding besides carbon atoms, 1, 2, 3 or 4 heteroatoms independently selected from the group consisting of N, O and S, for example, a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl; or

a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

Agriculturally acceptable salts of the inventive compounds encompass especially the salts of those cations or the acid addition salts of those acids whose cations and anions, respectively, have no adverse effect on the fungicidal action of said compounds. Suitable cations are thus in particular the ions of the alkali metals, preferably sodium and potassium, of the alkaline earth metals, preferably calcium, magnesium and barium, of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium. Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting such inventive compound with an acid of the corresponding anion, preferably of hydrochloric acid, hydrobromic acid, sulfuric acid, phosphoric acid or nitric acid. The inventive compounds can be present in atropisomers arising from restricted rotation about a single bond of asymmetric groups. They also form part of the subject matter of the present invention.

Depending on the substitution pattern, the compounds of formula I and their N-oxides may have one or more centers of chirality, in which case they are present as pure enantiomers or pure diastereomers or as enantiomer or diastereomer mixtures. Both, the pure enantiomers or diastereomers and their mixtures are subject matter of the present invention.

In the following, particular embodiments of the inventive compounds are described. Therein, specific meanings of the respective substituents are further detailled, wherein the meanings are in each case on their own but also in any combination with one another, particular embodiments of the present invention.

Furthermore, in respect of the variables, generally, the embodiments of the compounds I also apply to the intermediates.

R¹ according to the invention is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein

R^{x} is C₁-C₄-alkyl, C₁-C₄-haloalkyl, unsubstituted aryl or aryl that is substituted by one, two, three, four or five substituents R^{x1} independently selected from C₁-C₄-alkyl;

wherein the acyclic moieties of R¹ are unsubstituted or substituted with identical or different groups R^{1a} which independently of one another are selected from:

R^{1a} halogen, OH, CN, C₁-C₆-alkoxy, C₃-C₆-cycloalkyl, C₃-C₆-halocycloalky, C₁-C₄-haloalkoxy, C₁-C₆-alkylthio and phenoxy, wherein the phenyl group is unsubstituted or unsubstituted or substituted with R^{11a} selected from the group consisting of halogen, OH, C₁-C₄-alkyl, C₁-C₄-haloalkyl, C₁-C₄-alkoxy and C₁-C₄-haloalkoxy;

wherein the cycloalkyl, heteroaryl and aryl moieties of R¹ are unsubstituted or substituted with identical or different groups R^{1b} which independently of one another are selected from:

R^{1b} halogen, OH, CN, C₁-C₄-alkyl, C₁-C₄-alkoxy, C₁-C₄-haloalkyl, C₃-C₆-cycloalkyl, C₃-C₆-halocycloalkyl, C₁-C₄-haloalkoxy and C₁-C₆-alkylthio.

For every R¹ that is present in the inventive compounds, the following embodiments and preferences apply independently of the meaning of any other R¹ that may be present in the ring.

In one embodiment of formula I, R¹ is hydrogen.

In another embodiment of formula I, R¹ is halogen, in particular Br, F or Cl, more specifically F or Cl.

In still another embodiment of formula I, R¹ is OH.

In still another embodiment of formula I, R¹ is CN.

In still another embodiment of formula I R¹ is NH₂, NH(C₁-C₄-alkyl), N(C₁-C₄-alkyl)₂ or NH—SO₂-R^{x}, wherein R^{x} is C₁-C₄-alkyl, C₁-C₄-haloalkyl, unsubstituted aryl or aryl that is substituted by one, two, three, four or five substituents R^{x1} independently selected from C₁-C₄-alkyl.

In still another embodiment of formula I, R¹ is C₁-C₆-alkyl, in particular C₁-C₄-alkyl, such as CH₃.

In still another embodiment of formula I, R¹ is C₁-C₆-alkyl, in particular C₁-C₄-alkyl, such as CH₂CH₃.

In still another embodiment of formula I, R¹ is C₁-C₆-haloalkyl, in particular C₁-C₄-haloalkyl, such as CF₃, CHF₂, CH₂F, CCl₃, CHCl₂ or CH₂Cl.

In still another embodiment of formula I, R¹ is C₂-C₆-alkenyl or C₂-C₆-haloalkenyl, in particular C₂-C₄-alkenyl or C₂-C₄-haloalkenyl, such as CH═CH₂.

In still another embodiment of formula I, R¹ is C₂-C₆-alkynyl or C₂-C₆-haloalkynyl, in particular C₂-C₄-alkynyl or C₂-C₄-haloalkynyl, such as C≡CH.

In still another embodiment of formula I, R¹ is C₁-C₆-alkoxy, in particular C₁-C₄-alkoxy, more specifically C₁-C₂-alkoxy such as OCH₃ or OCH₂CH₃.

In still another embodiment of formula I, R¹ is C₁-C₆-haloalkoxy, in particular C₁-C₄-haloalkoxy, more specifically C₁-C₂-haloalkoxy such as OCF₃, OCHF₂, OCH₂F, OCCl₃, OCHCl₂ or OCH₂Cl, in particular OCF₃, OCHF₂, OCCl₃ or OCHCl₂.

In still another embodiment of formula I R¹ is C₃-C₆-cycloalkyl, in particular cyclopropyl. In still another embodiment of formula I, R¹ is C₃-C₆-cycloalkyl, for example cyclopropyl, substituted by one, two, three or up to the maximum possible number of identical or different groups

R^{1b} as defined and preferably herein.

In still another embodiment of formula I, R¹ is C₃-C₆-halocycloalkyl. In a special embodiment R¹ is fully or partially haloated cyclopropyl.

In still another embodiment of formula I, R¹ is unsubstituted aryl or aryl that is substituted by one, two, three or four R^{1b}, as defined herein. In particular, R¹ is unsubstituted phenyl or phenyl that is substituted by one, two, three or four R^{1b}, as defined herein.

In still another embodiment of formula I, R¹ is unsubstituted 5- or 6-membered heteroaryl.

According to still a further embodiment, R¹ is 5- or 6-membered heteroaryl that is substituted by one, two or three R^{1b}, as defined herein.

In still another embodiment of formula I, R¹ is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy and C₃-C₆-cycloalkyl; wherein the acyclic moieties of R¹ are not further substituted or carry one, two, three, four or five identical or different groups R^{1a} as defined below and wherein the cycloalkyl moieties of R¹ are not further substituted or carry one, two, three, four or five identical or different groups R^{1b} as defined below.

In still another embodiment of formula I, R¹ is independently selected from hydrogen, halogen, OH, C₁-C₆-alkyl, C₁-C₆-haloalkyl, C₁-C₆-alkoxy and C₁-C₆-haloalkoxy, in particular independently selected from F, Cl, Br, CN, OH, C₁-C₄-alkyl, C₁-C₄-haloalkyl, C₁-C₄-alkoxy and C₁-C₄-haloalkoxy. R^{1a} are the possible substituents for the acyclic moieties of R¹.

R^{1a} according to the invention is independently selected from halogen, OH, CN, C₁-C₆-alkoxy, C₃-C₆-cycloalkyl, C₃-C₆-halocycloalky, C₁-C₄-haloalkoxy, C₁-C₆-alkylthio and phenoxy, wherein the phenyl group is unsubstituted or unsubstituted or substituted with R^{11a} selected from the group consisting of halogen, OH, C₁-C₄-alkyl, C₁-C₄-haloalkyl, C₁-C₄-alkoxy and C₁-C₄-haloalkoxy, in particular selected from halogen, C₁-C₂-alkyl, C₁-C₂-haloalkyl, C₁-C₂-alkoxy and C₁-C₂-haloalkoxy, more specifically selected from halogen, such as F, Cl and Br. According to one embodiment R^{1a} is independently selected from halogen, OH, CN, C₁-C₂-alkoxy, C₃-C₆-cycloalkyl, C₃-C₆-halocycloalkyl and C₁-C₂-haloalkoxy. Specifically, R^{1a} is independently selected from F, Cl, OH, CN, C₁-C₂-alkoxy, cyclopropyl, 1-F-cyclopropyl, 1-Cl-cyclopropyl and C₁-C₂-haloalkoxy.

In one embodiment R^{1a} is independently selected from halogen, such as F, Cl, Br and I, more specifically F, Cl and Br.

In still another embodiment of formula I, R^{1a} is independently selected from OH, C₃-C₆-cycloalkyl, C₃-C₆-halocycloalky and C₁-C₂-haloalkoxy. Specifically, R^{1a} is independently selected from OH, cyclopropyl and C₁-C₂-haloalkoxy.

R^{1b} are the possible substituents for the cycloalkyl, heteroaryl and aryl moieties of R¹.

R^{1b} according to the invention is independently selected from halogen, OH, CN, C₁-C₄-alkyl, C₁-C₄-alkoxy, C₁-C₄-haloalkyl, C₃-C₆-cycloalkyl, C₃-C₆-halocycloalkyl and C₁-C₄-haloalkoxy.

According to one embodiment thereof R^{1b} is independently selected from halogen, CN, C₁-C₂-alkyl, C₁-C₂-alkoxy, C₁-C₂-haloalkyl, C₃-C₆-cycloalkyl, C₃-C₆-halocycloalkyl and C₁-C₂-haloalkoxy. Specifically, R^{1b} is independently selected from F, Cl, OH, CN, CH₃, OCH₃, cyclopropyl, 1-F-cyclopropyl, 1-Cl-cyclopropyl and halomethoxy.

In still another embodiment thereof R^{1b} is independently selected from C₁-C₂-alkyl, C₁-C₂-alkoxy, C₁-C₂-haloalkyl, C₃-C₆-cycloalkyl, C₃-C₆-halocycloalkyl, and C₁-C₂-haloalkoxy. Specifically, R^{1b} is independently selected from OH, CH₃, OCH₃, cyclopropyl, 1-F-cyclopropyl, 1-Cl-cyclopropyl and halomethoxy, more specifically independently selected from OH, CH₃, OCH₃, cyclopropyl, 1-F-cyclopropyl, 1-Cl-cyclopropyl and OCHF₂.

In still another embodiment R^{1b} is independently selected from halogen, such as F, Cl, Br and I, more specifically F, Cl and Br.

Particularly preferred embodiments of R¹ according to the invention are in Table P1 below, wherein each line of lines P1-1 to P1-16 corresponds to one particular embodiment of the invention. Thereby, for every R¹ that is present in the inventive compounds, these specific embodiments and preferences apply independently of the meaning of any other R¹ that may be present in the ring:

TABLE P1
“Ts” in the table stands for the tosylgroup SO₂-(p-CH₃)phenyl.

| No. | R¹ |
|---|---|
| P1-1 | H |
| P1-2 | Cl |
| P1-3 | F |
| P1-4 | Br |
| P1-5 | OH |
| P1-6 | CN |
| P1-7 | NO₂ |
| P1-8 | CH₃ |
| P1-9 | CH₂CH₃ |
| P1-10 | CF₃ |
| P1-11 | CHF₂ |
| P1-12 | OCH₃ |
| P1-13 | OCH₂CH₃ |
| P1-14 | OCF₃ |
| P1-15 | OCHF₂ |
| P1-16 | NH-Ts |

R² according to the invention is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein R^{x} is C₁-C₄-alkyl, C₁-C₄-haloalkyl, unsubstituted aryl or aryl that is substituted by one, two, three, four or five substituents Rx² independently selected from C₁-C₄-alkyl; wherein the acyclic moieties of R² are unsubstituted or substituted with identical or different groups Rea which independently of one another are selected from:

R^{2a} halogen, OH, CN, C₁-C₆-alkoxy, C₃-C₆-cycloalkyl, C₃-C₆-halocycloalkyl, C₁-C₄-haloalkoxy, C₁-C₆-alkylthio and phenoxy, wherein the phenyl group is unsubstituted or unsubstituted or substituted with R^{2a} selected from the group consisting of halogen, OH, C₁-C₄-alkyl, C₁-C₄-haloalkyl, C₁-C₄-alkoxy and C₁-C₄-haloalkoxy;

wherein the cycloalkyl, heteroaryl and aryl moieties of R² are unsubstituted or substituted with identical or different groups R^{2b} which independently of one another are selected from:

R^{2b} halogen, OH, CN, C₁-C₄-alkyl, C₁-C₄-alkoxy, C₁-C₄-haloalkyl, C₃-C₆-cycloalkyl, C₃-C₆-halocycloalkyl, C₁-C₄-haloalkoxy and C₁-C₆-alkylthio.

For every R² that is present in the inventive compounds, the following embodiments and preferences apply independently of the meaning of the other R² that may be present in the ring. In one embodiment of formula I, R² is halogen, in particular Br, F or Cl, more specifically F or Cl.

In still another embodiment of formula I, R² is hydrogen.

In still another embodiment of formula I, R² is OH.

In still another embodiment of formula I, R² is CN.

According to a further specific embodiment R² is NH₂, NH(C₁-C₄-alkyl), N(C₁-C₄-alkyl)₂ or NH—SO₂—R^{x}, wherein R^{x} is C₁-C₄-alkyl, C₁-C₄-haloalkyl, unsubstituted aryl or aryl that is substituted by one, two, three, four or five substituents Rx² independently selected from C₁-C₄-alkyl.

In still another embodiment of formula I, R² is C₁-C₆-alkyl, in particular C₁-C₄-alkyl, such as CH₃.

In still another embodiment of formula I, R² is C₁-C₆-alkyl, in particular C₁-C₄-alkyl, such as CH₂CH₃.

In still another embodiment of formula I, R² is C₁-C₆-haloalkyl, in particular C₁-C₄-haloalkyl, such as CF₃, CHF₂, CH₂F, CCl₃, CHCl₂ or CH₂Cl.

According to still a further embodiment, R² is C₂-C₆-alkenyl or C₂-C₆-haloalkenyl, in particular C₂-C₄-alkenyl or C₂-C₄-haloalkenyl, such as CH═CH₂.

According to still a further embodiment, R² is C₂-C₆-alkynyl or C₂-C₆-haloalkynyl, in particular C₂-C₄-alkynyl or C₂-C₄-haloalkynyl, such as C≡CH.

In still another embodiment of formula I, R² is C₁-C₆-alkoxy, in particular C₁-C₄-alkoxy, more specifically C₁-C₂-alkoxy such as OCH₃ or OCH₂CH₃.

In still another embodiment of formula I, R² is C₁-C₆-haloalkoxy, in particular C₁-C₄-haloalkoxy, more specifically C₁-C₂-haloalkoxy such as OCF₃, OCHF₂, OCH₂F, OCCl₃, OCHCl₂ or OCH₂Cl, in particular OCF₃, OCHF₂, OCCl₃ or OCHCl₂.

According to a further specific embodiment R² is C₃-C₆-cycloalkyl, in particular cyclopropyl.

In a further specific embodiment, R² is C₃-C₆-cycloalkyl, for example cyclopropyl, substituted by one, two, three or up to the maximum possible number of identical or different groups R^{2b} as defined and preferably herein.

In still another embodiment of formula I, R² is C₃-C₆-halocycloalkyl. In a special embodiment R² is fully or partially haloated cyclopropyl.

In still another embodiment of formula I, R² is unsubstituted aryl or aryl that is substituted by one, two, three or four R^{2b}, as defined herein. In particular, R² is unsubstituted phenyl or phenyl that is substituted by one, two, three or four R^{2b}, as defined herein.

In still another embodiment of formula I, R² is unsubstituted 5- or 6-membered heteroaryl.

According to still a further embodiment, R² is 5- or 6-membered heteroaryl that is substituted by one, two or three R²b, as defined herein.

In still another embodiment of formula I, R² is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy and C₃-C₆-cycloalkyl; wherein the acyclic moieties of R² are not further substituted or carry one, two, three, four or five identical or different groups R²a as defined below and wherein the cycloalkyl moieties of R² are not further substituted or carry one, two, three, four or five identical or different groups R²b as defined below.

In still another embodiment of formula I, R² is independently selected from hydrogen, halogen, OH, C₁-C₆-alkyl, C₁-C₆-haloalkyl, C₁-C₆-alkoxy and C₁-C₆-haloalkoxy, in particular independently selected from F, Cl, Br, CN, OH, C₁-C₄-alkyl, C₁-C₄-haloalkyl, C₁-C₄-alkoxy and C₁-C₄-halo-alkoxy.

R²a are the possible substituents for the acyclic moieties of R².

R²a according to the invention is independently selected from halogen, OH, CN, C₁-C₆-alkoxy, C₃-C₆-cycloalkyl, C₃-C₆-halocycloalky, C₁-C₄-haloalkoxy, C₁-C₆-alkylthio and phenoxy, wherein the phenyl group is unsubstituted or unsubstituted or substituted with Rea selected from the group consisting of halogen, OH, C₁-C₄-alkyl, C₁-C₄-haloalkyl, C₁-C₄-alkoxy and C₁-C₄-haloalkoxy, in particular selected from halogen, C₁-C₂-alkyl, C₁-C₂-haloalkyl, C₁-C₂-alkoxy and C₁-C₂-haloalkoxy, more specifically selected from halogen, such as F, Cl and Br.

According to one embodiment R²a is independently selected from halogen, OH, CN, C₁-C₂-alkoxy, C₃-C₆-cycloalkyl, C₃-C₆-halocycloalky and C₁-C₂-haloalkoxy. Specifically, Rea is independently selected from F, Cl, OH, CN, C₁-C₂-alkoxy, cyclopropyl, 1-F-cyclopropyl, 1-Cl-cyclopropyl and C₁-C₂-haloalkoxy.

In one embodiment R^{2a} is independently selected from halogen, such as F, Cl, Br and I, more specifically F, Cl and Br.

In still another embodiment of formula I, R^{2a} is independently selected from OH, C₃-C₆-cycloalkyl, C₃-C₆-halocycloalky and C₁-C₂-haloalkoxy. Specifically, Rea is independently selected from OH, cyclopropyl and C₁-C₂-haloalkoxy.

R^{2b} are the possible substituents for the cycloalkyl, heteroaryl and aryl moieties of R².

R^{2b} according to the invention is independently selected from halogen, OH, CN, C₁-C₄-alkyl, C₁-C₄-alkoxy, C₁-C₄-haloalkyl, C₃-C₆-cycloalkyl, C₃-C₆-halocycloalkyl and C₁-C₄-haloalkoxy.

According to one embodiment thereof R^{2b} is independently selected from halogen, CN, C₁-C₂-alkyl, C₁-C₂-alkoxy, C₁-C₂-haloalkyl, C₃-C₆-cycloalkyl, C₃-C₆-halocycloalkyl and C₁-C₂-haloalkoxy. Specifically, R^{2b} is independently selected from F, Cl, OH, CN, CH₃, OCH₃, cyclopropyl, 1-F-cyclopropyl, 1-Cl-cyclopropyl and halomethoxy.

In still another embodiment thereof R²b is independently selected from C₁-C₂-alkyl, C₁-C₂-alkoxy, C₁-C₂-haloalkyl, C₃-C₆-cycloalkyl, C₃-C₆-halocycloalkyl and C₁-C₂-haloalkoxy. Specifically, R^{2b} is independently selected from OH, CH₃, OCH₃, cyclopropyl, 1-F-cyclopropyl, 1-Cl-cyclopropyl and halomethoxy, more specifically independently selected from OH, CH₃, OCH₃, cyclopropyl, 1-F-cyclopropyl, 1-Cl-cyclopropyl and OCH F₂.

In still another embodiment R²b is independently selected from halogen, such as F, Cl, Br and I, more specifically F, Cl and Br.Particularly preferred embodiments of R² according to the invention are in Table P2 below, wherein each line of lines P2-1 to P2-16 corresponds to one particular embodiment of the invention. Thereby, for every R² that is present in the inventive compounds, these specific embodiments and preferences apply independently of the meaning of any other R² that may be present in the ring:

TABLE P2
“Ts” in the table stands for the tosylgroup SO₂-(p-CH₃)phenyl.

| No. | R² |
|---|---|
| P2-1 | H |
| P2-2 | Cl |
| P2-3 | F |
| P2-4 | Br |
| P2-5 | OH |
| P2-6 | CN |
| P2-7 | NO₂ |
| P2-8 | CH₃ |
| P2-9 | CH₂CH₃ |
| P2-10 | CF₃ |
| P2-11 | CHF₂ |
| P2-12 | OCH₃ |
| P2-13 | OCH₂CH₃ |
| P2-14 | OCF₃ |
| P2-15 | OCHF₂ |
| P2-16 | NH-Ts |

R³, R⁴ are independently selected from halogen, OH, CN, NO₂, SH, C₁-C₆-alkylthio, C₁-C₆-haloalkylthio, NH₂, NH(C₁-C₄-alkyl), N(C₁-C₄-alkyl)₂, NH—SO₂—R^{x}, C₁-C₆-alkyl, C₂-C₆-alkenyl, C₁-C₆-haloalkyl, C₂-C₆-alkynyl, C₂-C₆-haloalkynyl, C₁-C₆-alkoxy, C₁-C₆-haloalkoxy, CH(═O), C(═O)C₁-C₆-alkyl, C(═O)OC₁-C₆-alkyl, C(═O)NHC₁-C₆-alkyl, C(═O)N(C₁-C₆-alkyl)₂, CR^{x}═NR^{x}, a saturated or partially unsaturated three-, four-, five-, six-, seven-, eight-, nine-, or ten-membered carbocycle or heterocycle, wherein in each case one or two CH₂ groups of the carbo- and heterocycle may be replaced by a group independently selected from C(═O) and C(═S), five- or six-membered heteroaryl and aryl; wherein the heterocycle and the heteroaryl contain independently one, two, three or four heteroatoms selected from N, O and S;

wherein the acyclic moieties of R³ and R⁴ are independently unsubstituted or substituted with identical or different groups R^{1a} or R^{4a}, respectively, which independently of one another are selected from:

R^{3a}, R^{4a} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, C₁-C₆-alkoxy, C₃-C₆-cycloalkyl, C₃-C₆-halocycloalkyl, C₁-C₄-haloalkoxy, C₁-C₆-alkylthio, CH(═O), C(═O)C₁-C₆-alkyl, C(═O)OC₁-C₆-alkyl, C(═O)N alkyl, C(═O)N(C₁-C₆-alkyl)₂, CR^{x}═NR^{x}, a saturated or partially unsaturated three-, four-, five-, six-, seven-, eight-, nine-, or ten-membered carbocycle or heterocycle, wherein in each case one or two CH₂ groups of the carbo- and heterocycle may be replaced by a group independently selected from C(═O) and C(═S), aryl and phenoxy, wherein the aryl and phenyl groups are independently unsubstituted or substituted with substituents selected from the group consisting of halogen, OH, CN, NO₂, SH, NH₂, NH(C₁-C₄-alkyl), N(C₁-C₄-alkyl)2, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-haloalkyl, C₁-C₄-alkoxy and C₁-C₄-haloalkoxy;

wherein the carbocyclic, heterocyclic, heteroaryl and aryl moieties of R³ and R⁴ are independently unsubstituted or substituted with identical or different groups R^{3b} or R^{4b}, respectively, which independently of one another are selected from:

R^{3b}, R^{4b} halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-haloalkyl, C₃-C₆-cycloalkyl, C₃-C₆-halocycloalkyl, C₁-C₄-haloalkoxy, C₁-C₆-alkylthio, C₁-C₆-haloalkylthio, C₁-C₄-alkoxy-C₁-C₄-alkyl, phenyl and phenoxy, wherein the phenyl groups are unsubstituted or substituted with substituents selected from the group consisting of halogen, OH, C1-C₄-alkyl, C₁-C₄-haloalkyl, C₁-C₄-alkoxy and C₁-C₄-haloalkoxy;

and wherein R^{x} is as defined above;

In still another embodiment of formula I,R³ is selected from halogen, OH, CN, SH, C₁-C₆-alkylthio, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy and C₁-C₆-haloalkoxy, in particular halogen, OH, CN, C₁-C₄-alkylthio, C₁-C₆-alkyl, C₂-C₆-alkenyl, C₂-C₆-alkynyl, C₁-C₆-alkoxy and C₁-C₆-haloalkoxy, wherein R^{x} is defined below; and wherein the acyclic moieties of R³ are unsubstituted or substituted with identical or different groups R^{3a} as defined below.

In still another embodiment of formula I, R³ is selected from halogen, OH, CN, SH, C₁-C₆-alkylthio, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy and C₁-C₆-haloalkoxy, in particular halogen, OH, CN, C₁-C₄-alkylthio, C₁-C₆-alkyl, C₂-C₆-alkenyl, C₂-C₆-alkynyl, C₁-C₆-alkoxy and C₁-C₆-haloalkoxy, wherein R^{x} is defined below; and wherein the acyclic moieties of R³ are unsubstituted or substituted with identical or different groups R^{3a} as defined below.

In still another embodiment of formula I, R³ is C₁-C₆-alkyl, such as CH₃, C₂H₅, n-propyl, i-propyl, n-butyl, i-butyl, tert-butyl, n-pentyl or i-pentyl.

In still another embodiment of formula I, R³ is C₁-C₆-alkyl, in particular C₁-C₄-alkyl, more specifically C₁-C₂-alkyl such as CH₃,

In still another embodiment of formula I, R³ is C₁-C₆-haloalkyl, in particular C₁-C₄-haloalkyl, more specifically C₁-C₂-haloalkyl, such as CF₃, CCl₃, FCH₂, ClCH₂, F₂CH, Cl₂CH, CF₃CH₂, CCl₃CH₂ or CF₂CH F₂.

In still another embodiment of formula I, R³ is C₁-C₆-haloalkyl, in particular C₁-C₄-haloalkyl, more specifically C₁-C₂-haloalkyl such as FCH₂.

In still another embodiment of formula I, R³ is C₁-C₆-haloalkyl, in particular C₁-C₄-haloalkyl, more specifically C₁-C₂-haloalkyl such as F₂CH.

In still another embodiment of formula I, R³ is C₁-C₆-haloalkyl, in particular C₁-C₄-haloalkyl, more specifically C₁-C₂-haloalkyl such as CF₃.

In still another embodiment of formula I, R³ is phenyl-C₁-C₆-alkyl, such as phenyl-CH₂, wherein the phenyl moiety in each case is unsubstituted or substituted by one, two or three identical or different groups R^{3b} which independently of one another are selected from halogen, C₁-C₂-alkyl, C₁-C₂-alkoxy, C₁-C₂-haloalkyl and C₁-C₂-haloalkoxy, in particular F, Cl, Br, CH₃, OCH₃, CF₃ and OCF₃.

In still another embodiment of formula I, R³ is aryl, in particular phenyl, wherein the aryl or phenyl moiety in each case is unsubstituted or substituted by identical or different groups R^{3b} which independently of one another are selected from halogen, C₁-C₂-alkyl, C₁-C₂-alkoxy, C₁-C₂-haloalkyl and C₁-C₂-haloalkoxy, in particular F, Cl, Br, CH₃, OCH₃, CF₃ and OCF₃. In one embodiment, R³ is unsubstituted phenyl. In another embodiment, R³ is phenyl, that is substituted by one, two or three, in particular one, halogen, in particular selected from F, Cl and Br, more specifically selected from F and Cl.

In still another embodiment of formula I, R³ is a partially unsaturated three-, four-, five-, six-, seven-, eight-, nine-, or ten-membered carbocycle or heterocycle, in particular three-, four-, five- or six-membered, wherein the heterocycle contains one, two, three or four heteroatoms selected from N, O and S, and wherein the carbocycle and heterocycle are unsubstituted or substituted with substituents R^{3b} as defined below. According to one embodiment thereof, the carbocycle or heterocycle is unsubstituted.

According to still a further embodiment, R³ is a saturated three-, four-, five-, six-, seven-, eight-, nine-, or ten-membered carbocycle or heterocycle, in particular three-, four-, five- or six-membered, wherein the heterocycle contains one, two, three or four heteroatoms selected from N, O and S, and wherein the carbocycle and heterocycle are unsubstituted or substituted with substituents R^{3b} as defined below. According to one embodiment thereof, the carbocycle or heterocycle is unsubstituted.

In still another embodiment of formula I, R³ is a partially unsaturated three-, four-, five-, six-, seven-, eight-, nine-, or ten-membered heterocycle, in particular three-, four-, five- or six-membered, wherein the heterocycle contains one, two, three or four heteroatoms selected from N, O and S, and wherein the heterocycle is unsubstituted or substituted bysubstituents R^{3b} as defined below. According to one embodiment thereof, the heterocycle is unsubstituted.

In still another embodiment of formula I, R³ is a saturated three-, four-, five-, six-, seven-, eight-, nine-, or ten-membered heterocycle, in particular three-, four-, five- or six-membered, wherein the heterocycle contains one, two, three or four heteroatoms selected from N, O and S, and wherein the heterocycle is unsubstituted or substituted bysubstituents R^{3b} as defined below.

According to one embodiment thereof, the heterocycle is unsubstituted.

In still another embodiment of formula I, in the embodiments of R³ described above, the heterocycle contains preferably one, two or three, more specifically one or two heteroatoms selected from N, O and S. More specifically, the hetereocycle contains one heteroatom selected from N, O and S. In particular, the heterocycle contains one or two, in particular one O.

In one embodiment, R³ is a 4-membered saturated heterocycle which contains 1 or 2 heteroatoms, in particular 1 heteroatom, from the group consisting of N, O and S, as ring members. In one embodiment, the heterocycle contains one O as heteroatom. For example, the formed heterocycle is oxetane. According to one embodiment thereof, the heterocycle is unsubstituted, i.e. it does not carry any substituent R^{3b}. In still another embodiment of formula I, it is substituted by R^{3b}.

In still another embodiment of formula I, R³ is a 5-membered saturated heterocycle which contains 1, 2 or 3, in particular 1 or 2, heteroatoms from the group consisting of N, O and S, as ring members. In one embodiment, the heterocycle contains one 0 as heteroatom. According to one embodiment thereof, the heterocycle is unsubstituted, i.e. it does not carry any substituent R^{3b}.

In still another embodiment of formula I, it is substituted by R^{3b}.

In still another embodiment of formula I, R³ is a 6-membered saturated heterocycle which contains 1, 2 or 3, in particular 1 or 2, heteroatoms from the group consisting of N, O and S as ring members. According to one embodiment thereof, the heterocycle is unsubstituted, i.e. it does not carry any substituent R^{3b}. In still another embodiment of formula I, it is substituted by R^{3b}.

According to one specific embodiment thereof, said 6-membered saturated heterocycle contains 1 or 2, in particular 1, heteroatom(s) O. According to one embodiment thereof, the respective 6-membered heterocycle is unsubstituted, i.e. it does not carry any substituent R^{3b}. In still another embodiment of formula I, it is substituted by R^{3b}.

In still another embodiment of formula I, R³ is a partially unsaturated three-, four-, five-, six-, seven-, eight-, nine-, or ten-membered carbocycle, in particular three-, four-, five- or six-membered, wherein the carbocycle is unsubstituted or substituted by substituents R^{3b} as defined below. According to one embodiment thereof, the carbocycle is unsubstituted. In still another embodiment of formula I, R³ is a saturated three-, four-, five-, six-, seven-, eight-, nine-, or ten-membered carbocycle, in particular three-, four-, five- or six-membered, wherein the carbocycle is unsubstituted or substituted bysubstituents R^{3b} as defined below. According to one embodiment thereof, the carbocycle is unsubstituted.

In one embodiment, R³ is a 3-membered saturated carbocycle. According to one embodiment thereof, the carbocycle is unsubstituted, i.e. it does not carry any substituent R^{3b}. In still another embodiment of formula I, it is substituted by R^{3b}.

In one embodiment, R³ is a 4-membered saturated carbocycle. According to one embodiment thereof, the carbocycle is unsubstituted, i.e. it does not carry any substituent R^{3b}. In still another embodiment of formula I, it is substituted by R^{3b}.

In one embodiment, R³ is a 5-membered saturated carbocycle. According to one embodiment thereof, the carbocycle is unsubstituted, i.e. it does not carry any substituent R^{3b}. In still another embodiment of formula I, it is substituted by R^{3b}.

In one embodiment, R³ is a 6-membered saturated carbocycle. According to one embodiment thereof, the carbocycle is unsubstituted, i.e. it does not carry any substituent R^{3b}. In still another embodiment of formula I, it is substituted by R^{3b}.

In still another embodiment of formula I, R³ is selected from C₁-C₆-alkyl, C₁-C₆-haloalkyl, phenyl-C₁-C₆-alkyl, halogenphenyl-C₁-C₆-alkyl, phenyl, halogenphenyl and three-, four-, five- or six-membered carbocycle, wherein the carbocycle is unsubstituted or is substituted by substituents R^{3b} as defined below. According to one embodiment thereof, the carbocycle is unsubstituted. In a particular embodiment, R³ is selected from C₁-C₆-alkyl, C₁-C₆-haloalkyl, phenyl-CH₂, halogenphenyl-CH₂, phenyl, halogenphenyl and three-, four-, five- or six-membered carbocycle, wherein the carbocycle is unsubstituted or it is substituted by substituents R^{3b} as defined below.

In still another embodiment of formula I, R³ is selected from C₁-C₆-alkyl, C₁-C₆-haloalkyl, phenyl-C₁-C₆-alkyl, halogenphenyl-C₁-C₆-alkyl, phenyl, halogenphenyl and three-, four-, five- or six-membered carbocycle, wherein the carbocycle is unsubstituted or substituted by R^{3b} as defined below. According to one embodiment thereof, the carbocycle is unsubstituted. In a particular embodiment, R³ is selected from C₁-C₆-alkyl, C₁-C₆-haloalkyl, phenyl-CH₂, halogenphenyl-CH₂, phenyl, halogenphenyl and three-, four-, five- or six-membered carbocycle, wherein the carbocycle is unsubstituted or it is substituted by substituents R^{3b} as defined below.

Particularly preferred embodiments of R³ according to the invention are in Table P3 below, wherein each line of lines P3-1 to P3-33 corresponds to one particular embodiment of the invention. The connection point to the carbon atom, to which R³ is bound is marked with “#” in the drawings.

TABLE P3
(py = pyridyl):

| No. | R³ |
|---|---|
| P3-1 | CN |
| P3-2 | CH₃ |
| P3-3 | C₂H₅ |
| P3-4 | CH₂CH₂CH₃ |
| P3-5 | CH(CH₃)₂ |
| P3-6 | CH₂CH₂CH₂CH₃ |
| P3-7 | CH₂CH(CH₃)₂ |
| P3-8 | C(CH₃)₃ |
| P3-9 | CH₂CH₂CH₂CH₂CH₃ |
| P3-10 | CH₂CH₂CH(CH₃)₂ |
| P3-11 | CF₃ |
| P3-12 | CHF₃ |
| P3-13 | CHCl₂ |
| P3-14 | CH₂F |
| P3-15 | CH₂Cl |
| P3-16 | CH₂CF₃ |
| P3-17 | CH₂CCl₃ |
| P3-18 | CF₂CHF₂ |
| P3-19 | C₆H₅ |
| P3-20 | 4-Cl—C₆H₄ |
| P3-21 | 4-F—C₆H₄ |
| P3-22 | CH₂—C₆H₅ |
| P3-23 | 3-py |
| P3-24 | 2-py |
| P3-25 | 4-py |
| P3-26 | 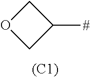 |
| P3-27 | 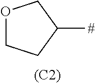 |
| P3-28 | 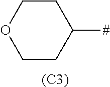 |
| P3-29 | 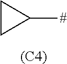 |
| P3-30 | 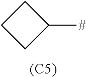 |
| P3-31 | 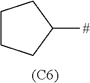 |
| P3-32 | 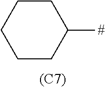 |
| P3-33 | 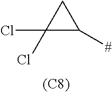 |

In still another embodiment of formula I,R⁴ is selected from halogen, OH, CN, SH, C₁-C₆-alkylthio, C₁-C₆-haloalkylthio, NH₂, NH(C₁-C₄-alkyl), N(C₁-C₄-alkyl)₂, NH—SO₂—R^{x}, C₁-C₆-alkyl, I, C₁-C₆-haloalkyl, C₂-C₆-alkenyl, C₂-C₆-haloalkenyl, C₂-C₆-alkynyl, C₂-C₆-haloalkynyl, C₁-C₆-alkoxy and C₁-C₆-haloalkoxy, CH(═O), C(═O)C₁-C₆-alkyl, C(═O)OC₁-C₆-alkyl, C(═O)NHC₁-C₆-alkyl, C(═O)N(C₁-C₆-alkyl)₂, CR^{x}═NR^{x}, in particular hydrogen, halogen, OH, CN, C₁-C₄-alkylthio, C₁-C₆-alkyl, C₂-C₆-alkenyl, C₂-C₆-alkynyl, C₁-C₆-alkoxy and C₁-C₆-haloalkoxy,more preferably CN, C₁-C₆-alkyl, C₂-C₆-alkenyl, C₂-C₆-alkynyl, C₁-C₆-alkoxy and C₁-C₆-haloalkoxy; wherein R^{x} is defined below; and wherein the acyclic moieties of R⁴ are unsubstituted or substituted with identical or different groups R^{4a} as defined below.

In still another embodiment of formula I, R⁴ is selected from halogen, OH, CN, SH, C₁-C₆-alkylthio, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy and C₁-C₆-haloalkoxy, in particular halogen, OH, CN, C₁-C₄-alkylthio, Cr C₆-alkyl, C₂-C₆-alkenyl, C₂-C₆-alkynyl, C₁-C₆-alkoxy and C₁-C₆-haloalkoxy, wherein R^{x} is defined below; and wherein the acyclic moieties of R⁴ are unsubstituted or substituted with identical or different groups R^{4a} as defined below.

In still another embodiment of formula I, R⁴ is C₁-C₆-alkyl, such as CH₃, C₂H₅, n-propyl, i-propyl, n-butyl, i-butyl, tert-butyl, n-pentyl or i-pentyl.

In still another embodiment of formula I, R⁴ is C₁-C₆-alkyl, in particular C₁-C₄-alkyl, more specifically C₁-C₂-alkyl such as CH₃.

In still another embodiment of formula I, R⁴ is C₁-C₆-alkyl, in particular C₁-C₄-alkyl, more specifically C₁-C₂-alkyl such as CH₂CH₃.

In still another embodiment of formula I, R⁴ is C₁-C₆-haloalkyl, in particular C₁-C₄-haloalkyl, more specifically C₁-C₂-haloalkyl, such as CF₃, CCl₃, FCH₂, ClCH₂, F₂CH, Cl₂CH, CF₃CH₂, CCl₃CH₂ or CF₂CH F₂.

In still another embodiment of formula I, R⁴ is C₁-C₆-haloalkyl, in particular C₁-C₄-haloalkyl, more specifically C₁-C₂-haloalkyl such as FCH₂.

In still another embodiment of formula I, R⁴ is C₁-C₆-haloalkyl, in particular C₁-C₄-haloalkyl, more specifically C₁-C₂-haloalkyl such as F₂CH.

In still another embodiment of formula I, R⁴ is C₁-C₆-haloalkyl, in particular C₁-C₄-haloalkyl, more specifically C₁-C₂-haloalkyl such as CF₃.

According to a further specific embodiment of formula I, R⁴ is CH(═O), C(═O)C₁-C₆-alkyl, C(═O)O(C₁-C₆-alkyl), C(═O)NH(C₁-C₆-alkyl) or C(═O)N(C₁-C₆-alkyl)₂, wherein alkyl is CH₃, C₂H₅, n-propyl, i-propyl, n-butyl, i-butyl, tert-butyl, n-pentyl or i-pentyl.

According to a further specific embodiment of formula I, R⁴ is C₁-C₄-alkyl-CH(═O), C₁-C₄-alkyl-C(═O)C₁-C₆-alkyl, C₁-C₄-alkyl-C(═O)O(C₁-C₆-alkyl), C₁-C₄-alkyl-C(═O)NH(C₁-C₆-alkyl) or C₁-C₄-alkyl-C(═O)N(C₁-C₆-alkyl)₂, especially CH₂CH(═O), CH₂C(═O)C₁-C₆-alkyl, CH₂C(═O)O(C₁-C₆-alkyl), CH₂C(═O)NH(C₁-C₆-alkyl) or CH₂C(═O)N(C₁-C₆-alkyl)₂ wherein alkyl is CH₃, C₂H₅, n-propyl, i-propyl, n-butyl, i-butyl, tert-butyl, n-pentyl or i-pentyl.

In still another embodiment of formula I, R⁴ is CR^{x}═NR^{x}, such as C(CH₃)═N—OCH₃, C(CH₃)═N—OCF₃ and C(CH₃)═N—OCH₂CH₃,

In still another embodiment of formula I, R⁴ is C₁-C₆-alkylOH, in particular C₁-C₄-haloalkylOH, more specifically HO—C₁-C₂-alkyl, such as CH₂OH.

In still another embodiment of formula I, R⁴ is CN.

In still another embodiment of formula I, R⁴ is C₁-C₆-alkyl-CN, in particular C₁-C₄-haloalkyl-CN, more specifically C₁-C₂-alkyl-CN, such as CH₂CN.

In still another embodiment of formula I, R⁴ is C₁-C₆-alkylthio, in particular C₁-C₃-alkylthio, more specifically C₁-C₃-alkylthio, such as CH₂SCH₃ or CH₂SCH₂CH₃.

In still another embodiment of formula I, R⁴ is C₁-C₆-alkylNH(C₁-C₄-alkyl), in particular C₁-C₄-alkylNH(C₁-C₃-alkyl), more specifically C₁-C₂-alkylNH(C₁-C₂-alkyl), such as CH₂NHCH₃.

In still another embodiment of formula I, R⁴ is C₁-C₆-alkylN(C₁-C₄-alkyl)2, in particular C₁-C₄-alkylN(C₁-C₃-alkyl)₂, more specifically C₁-C₂-alkylN(C₁-C₂-alkyl)₂, such as CH₂N(CH₃)₃.

In still another embodiment of formula I, R⁴ is C₁-C₆-alkoxy, in particular C₁-C₄-alkoxy, more specifically C₁-C₂-alkoxy, such as OCH₃, OCH₂CH₃.

According to a further specific embodiment of formula I, R⁴ is C₁-C₆-alkyl-C₁-C₆-alkoxy, in particular C₁-C₄-alkyl-C₁-C₄-alkoxy, more specifically C₁-C₂-alkyl-C₁-C₂-alkoxy, such as CH₂OCH₃ or CH₂OCH₂CH₃.

According to a further specific embodiment of formula I, R⁴ is C₂-C₆-alkenyloxy, in particular C₂-C₄-alkenyloxy, more specifically C₁-C₂-alkenyloxy such as OCH═CH₂, OCH₂CH═CH₂OC(CH₃)CH═CH₂, CH₂OCH═CH₂, or CH₂OCH₂CH═CH₂. According to a further specific embodiment of formula I, R⁴ is C₂-C₆-alkynyloxy, in particular C₂-C₄-alkynyloxy, more specifically C₁-C₂-alkynyloxy such as OC≡CH.

In still another embodiment of formula I, R⁴ is C₁-C₆-haloalkoxy, in particular C₁-C₄-haloalkoxy, more specifically C₁-C₃-haloalkoxy, such as OCH₂F, OCHF₂, OCF₃, CH₂OCH₂CF₃, CH₂OCH₂CH₂F, CH₂OCH₂CHF₂, CH₂OCF₂CF₃, CH₂OCF₂CH₂F, CH₂OCF₂CHF₂, OCCl₃ or OCHCl₂.

According to a further specific embodiment of formula I, R⁴ is C₁-C₆-alkyl-C₁-C₆-halogenalkoxy, in particular C₁-C₄-alkyl-C₁-C₄-halogenalkoxy, more specifically C₁-C₂-alkyl-C₁-C₂-halogenalkoxy such as CH₂OCF₃, CH₂OCHF₂, CH₂OCH₂F, CH₂OCCl₃, CH₂OCHCl₂ or CH₂OCH₂Cl, in particular CH₂OCF₃, CH₂OCHF₂, CH₂OCCl₃ or CH₂OCHCl₂.

In still another embodiment of formula I, R⁴ is C₁-C₆-alkenyl, in particular C₁-C₄-alkenyl, more specifically C₁-C₂-alkenyl, such as CH═CH₂.

In still another embodiment of formula I, R⁴ is C₁-C₆-alkenyl, in particular C₁-C₄-alkenyl, more specifically C₁-C₃-alkenyl, such as CH₂CH═CH₂ and C(CH₃)═CH₂.

In still another embodiment of formula I, R⁴ is C₁-C₆-haloalkenyl, in particular C₁-C₄-haloalkenyl, more specifically C₁-C₂-haloalkenyl, such as CH═CCl₂, CCl═CCl₂, CH═CF₂ and CF═CF₂.

In still another embodiment of formula I, R⁴ is C₁-C₆-haloalkenyl, in particular C₁-C₄-haloalkenyl, more specifically C₁-C₃-haloalkenyl, such as CH₂—CH═CCl₂, CH₂—CCl=CCl₂, CH₂—CH═CF₂, CH₂—CF═CF₂, CCl₂—CH═CCl₂, CCl₂—CCl=CCl₂, CF₂—CH═CF₂ and CF₂—CF═CF₂.

According to still a further embodiment of formula I, R⁴ is C₂-C₆-cycloalkenyl, in particular C₂-C₄-cycloalkenyl, such as CH═CH₂—CPr.

In still another embodiment of formula I, R⁴ is C₁-C₆-alkynyl, in particular C₁-C₄-alkynyl, more specifically C₁-C₂-alkynyl, such as C≡CH.

In still another embodiment of formula I, R⁴ is C₁-C₆-alkynyl, in particular C₁-C₄-alkynyl, more specifically C₁-C₃-alkynyl, such as CH₂—C≡CH.

In still another embodiment of formula I, R⁴ is C₁-C₆-alkynyl, in particular C₁-C₄-alkynyl, such as CH₂—C≡C—CH₃.

In still another embodiment of formula I, R⁴ is C₁-C₆-haloalkynyl, in particular C₁-C₄-haloalkynyl, more specifically C₁-C₂-haloalkynyl, such as C≡CCl.

In still another embodiment of formula I, R⁴ is C₁-C₆-haloalkynyl, in particular C₁-C₄-haloalkynyl, more specifically C₁-C₃-haloalkynyl, such as CH₂—C≡CCl and C≡C—CF₃.

In still another embodiment of formula I, R⁴ is C₁-C₆-haloalkynyl, in particular C₁-C₄-haloalkynyl, more specifically C₁-C₄-haloalkynyl, such as CH₂—C≡C—CF₃.

In still another embodiment of formula I, R⁴ is C₁-C₆-cycloalkynyl, in particular C₁-C₄-cycloalkynyl, more specifically C₁-C₂-cycloalkynyl, such as C≡C—CPr.

In still another embodiment of formula I, R⁴ is C₁-C₆-cycloalkynyl, in particular C₁-C₄-cycloalkynyl, more specifically C₁-C₃-cycloalkynyl, such as CH₂—C≡C—CPr.

According to still another embodiment of formula I, R⁴ is selected from C₁-C₆-alkyl which is substituted, a saturated three-, four-, five-, six-, seven-, eight-, nine-, or ten-membered carbocycle, in particular three-, four-, five- or six-membered, wherein the carbocycle is unsubstituted or substituted by substituents Rob as defined below. According to one embodiment thereof, the carbocycle is unsubstituted.

According to one embodiment, R⁴ is selected from C₁-C₆-alkyl which is substituted, a 3-membered saturated carbocycle. According to one embodiment thereof, the carbocycle is unsubstituted, i.e. it does not carry any substituent R^{4b}. According to still another embodiment of formula I, it is substituted by R^{4b}.

According to one embodiment, R⁴ is selected from C₁-C₆-alkyl which is substituted, a 4-membered saturated carbocycle. According to one embodiment thereof, the carbocycle is unsubstituted, i.e. it does not carry any substituent R^{4b}. According to still another embodiment of formula I, it is substituted by R^{4b}.

According to one embodiment, R⁴ is selected from C₁-C₆-alkyl which is substituted, a 5-membered saturated carbocycle. According to one embodiment thereof, the carbocycle is unsubstituted, i.e. it does not carry any substituent R^{4b}. According to still another embodiment of formula I, it is substituted by R^{4b}.

According to one embodiment, R⁴ is selected from C₁-C₆-alkyl which is substituted, a 6-membered saturated carbocycle. According to one embodiment thereof, the carbocycle is unsubstituted, i.e. it does not carry any substituent R^{4b}. According to still another embodiment of formula I, it is substituted by R^{4b}.

According to a further specific embodiment of formula I, R⁴ is C₁-C₆-alkyl-NH(C₁-C₄-alkyl) or C₁-C₆-alkyl-N(C₁-C₄-alkyl)₂, wherein alkyl is CH₃, C₂H₅, n-propyl, i-propyl, n-butyl, i-butyl, tert-butyl, n-pentyl or i-pentyl.

In still another embodiment of formula I, R⁴ is selected from C₁-C₆-alkyl which is substituted, a C₃-C₆-cycloalkyl, in particular C₃-C₄-cycloalkyl, more specifically C₃-C₄-cycloalkyl, such as CH₂CPr.

In still another embodiment of formula I, R⁴ is selected from C₁-C₆-alkyl which is substituted, a C₃-C₆-halocycloalkyl, in particular C₃-C₄-halocycloalkyl, more specifically C₃-C₄-halocycloalkyl-C₁-C₂-alkyl, such as CH₂—C₃H₂F₂ or CH₂—C₃H₂Cl₂.

According to a further specific embodiment of formula I, R⁴ is C₁-C₆-alkylheterocycle, especially CH₂ substituted by a 4-membered saturated heterocycle which contains 1 or 2 heteroatoms, in particular 1 heteroatom, from the group consisting of N, O and S, as ring members. According to one embodiment, the heterocycle contains one 0 as heteroatom. For example, the formed heterocycle is oxetane. According to one embodiment thereof, the heterocycle is unsubstituted, i.e. it does not carry any substituent R^{4b}. According to still another embodiment of formula I, it is substituted by R^{4b}.

According to a further specific embodiment of formula I, R⁴ is C₁-C₆-alkylheterocycle, especially CH₂ substituted by a 5-membered saturated heterocycle which contains 1, 2 or 3, in particular 1 or 2, heteroatoms from the group consisting of N, O and S, as ring members. According to one embodiment, the heterocycle contains one O as heteroatom. According to one embodiment thereof, the heterocycle is unsubstituted, i.e. it does not carry any substituent R^{4b}. According to still another embodiment of formula I, it is substituted by R^{4b}.

According to a further specific embodiment of formula I, R⁴ is C₁-C₆-alkylheterocycle, especially CH₂ subsitited by a 6-membered saturated heterocycle which contains 1, 2 or 3, in particular 1 or 2, heteroatoms from the group consisting of N, O and S as ring members. According to one embodiment thereof, the heterocycle is unsubstituted, i.e. it does not carry any substituent R^{4b}.

According to still another embodiment of formula I, it is substituted by R^{4b}. According to one specific embodiment thereof, said 6-membered saturated heterocycle contains 1 or 2, in particu-lar 1, heteroatom(s) O. According to one embodiment thereof, the respective 6-membered heterocycle is unsubstituted, i.e. it does not carry any substituent R^{4b}. According to still another embodiment of formula I, it is substituted by R^{4b}.

According to a further specific embodiment of formula I, R⁴ is C₁-C₆-alkylheterocycle, especially CH₂ substituted by a 5-membered saturated heterocycle which contains one N as ring member and optionally one or two groups CH₂ are replaced by C(═O).

In still another embodiment of formula I, R⁴ is phenyl-C₁-C₆-alkyl, such as phenyl-CH₂, wherein the phenyl moiety in each case is unsubstituted or substituted by one, two or three identical or different groups R^{4b} which independently of one another are selected from CN, halogen, C₁-C₂-alkyl, C₁-C₂-alkoxy, C₁-C₂-haloalkyl and C₁-C₂-haloalkoxy, in particular CN, F, Cl, Br, CH₃, CHF₂, OCH₃, OCHF₂, CF₃ and OCF₃.

In still another embodiment of formula I, R⁴ is unsubstituted phenyl-CH₂. In still another embodiment of formula I, R⁴ is phenyl-CH₂, that is substituted by one, two or three, in particular one, halogen, in particular selected from F, Cl and Br, more specifically selected from F and Cl.

In still another embodiment of formula I, R⁴ is aryl, in particular phenyl, wherein the aryl or phenyl moiety in each case is unsubstituted or substituted by identical or different groups R^{4b} which independently of one another are selected from CN, halogen, C₁-C₂-alkyl, C₁-C₂-alkoxy, C₁-C₂-haloalkyl and C₁-C₂-haloalkoxy, in particular CN, F, Cl, Br, CH₃, OCH₃, OCHF₂, CF₃, CHF₂ and OCF₃. In one embodiment, R⁴ is unsubstituted phenyl. In another embodiment, R⁴ is phenyl, that is substituted by one, two or three, in particular one, halogen, in particular selected from F, Cl and Br, more specifically selected from F and Cl.

In still another embodiment of formula I, R⁴ is unsubstituted phenyl.

In still another embodiment of formula I, R⁴ is phenyl, that is substituted by one, two or three, in particular one, halogen, in particular selected from F, Cl and Br, more specifically selected from F and Cl.

In still another embodiment of formula I, R⁴ is a partially unsaturated three-, four-, five-, six-, seven-, eight-, nine-, or ten-membered carbocycle or heterocycle, in particular three-, four-, five- or six-membered, wherein the heterocycle contains one, two, three or four heteroatoms selected from N, O and S, and wherein the carbocycle and heterocycle are unsubstituted or substituted with substituents R^{4b} as defined below. According to one embodiment thereof, the carbocycle or heterocycle is unsubstituted.

According to still a further embodiment, R⁴ is a saturated three-, four-, five-, six-, seven-, eight-, nine-, or ten-membered carbocycle or heterocycle, in particular three-, four-, five- or six-membered, wherein the heterocycle contains one, two, three or four heteroatoms selected from N, O and S, and wherein the carbocycle and heterocycle are unsubstituted or substituted with substituents R^{4b} as defined below. According to one embodiment thereof, the carbocycle or heterocycle is unsubstituted.

In still another embodiment of formula I, R⁴ is a partially unsaturated three-, four-, five-, six-, seven-, eight-, nine-, or ten-membered heterocycle, in particular three-, four-, five- or six-membered, wherein the heterocycle contains one, two, three or four heteroatoms selected from N, O and S, and wherein the heterocycle is unsubstituted or substituted by substituents R^{4b} as defined below. According to one embodiment thereof, the heterocycle is unsubstituted.

In still another embodiment of formula I, R⁴ is a saturated three-, four-, five-, six-, seven-, eight-, nine-, or ten-membered heterocycle, in particular three-, four-, five- or six-membered, wherein the heterocycle contains one, two, three or four heteroatoms selected from N, O and S, and wherein the heterocycle is unsubstituted or substituted by substituents R^{4b} as defined below.

According to one embodiment thereof, the heterocycle is unsubstituted.

In still another embodiment of formula I, in the embodiments of R⁴ described above, the heterocycle contains preferably one, two or three, more specifically one or two heteroatoms selected from N, O and S. More specifically, the hetereocycle contains one heteroatom selected from N, O and S. In particular, the heterocycle contains one or two, in particular one O.

In one embodiment, R⁴ is a 4-membered saturated heterocycle which contains 1 or 2 heteroatoms, in particular 1 heteroatom, from the group consisting of N, O and S, as ring members. In one embodiment, the heterocycle contains one O as heteroatom. For example, the formed heterocycle is oxetane. According to one embodiment thereof, the heterocycle is unsubstituted, i.e. it does not carry any substituent R^{4b}. In still another embodiment of formula I, it is substituted by R^{4b}.

In still another embodiment of formula I, R⁴ is a 5-membered saturated heterocycle which contains 1, 2 or 3, in particular 1 or 2, heteroatoms from the group consisting of N, O and S, as ring members. In one embodiment, the heterocycle contains one O as heteroatom. According to one embodiment thereof, the heterocycle is unsubstituted, i.e. it does not carry any substituent R^{4b}.

In still another embodiment of formula I, it is substituted by R^{4b}.

In still another embodiment of formula I, R⁴ is a 6-membered saturated heterocycle which contains 1, 2 or 3, in particular 1 or 2, heteroatoms from the group consisting of N, O and S as ring members. According to one embodiment thereof, the heterocycle is unsubstituted, i.e. it does not carry any substituent R^{4b}. In still another embodiment of formula I, it is substituted by R^{4b}.

According to one specific embodiment thereof, said 6-membered saturated heterocycle contains 1 or 2, in particular 1, heteroatom(s) O. According to one embodiment thereof, the respective 6-membered heterocycle is unsubstituted, i.e. it does not carry any substituent R^{4b}. In still another embodiment of formula I, it is substituted by R^{4b}.

In still another embodiment of formula I, R⁴ is a partially unsaturated three-, four-, five-, six-, seven-, eight-, nine-, or ten-membered carbocycle, in particular three-, four-, five- or six-membered, wherein the carbocycle is unsubstituted or substituted by substituents R^{4b} as defined below. According to one embodiment thereof, the carbocycle is unsubstituted.

In still another embodiment of formula I, R⁴ is a saturated three-, four-, five-, six-, seven-, eight-, nine-, or ten-membered carbocycle, in particular three-, four-, five- or six-membered, wherein the carbocycle is unsubstituted or substituted by substituents R^{4b} as defined below. According to one embodiment thereof, the carbocycle is unsubstituted.

In one embodiment, R⁴ is a 3-membered saturated carbocycle. According to one embodiment thereof, the carbocycle is unsubstituted, i.e. it does not carry any substituent R^{4b}. In still another embodiment of formula I, it is substituted by R^{4b}.

In one embodiment, R⁴ is a 4-membered saturated carbocycle. According to one embodiment thereof, the carbocycle is unsubstituted, i.e. it does not carry any substituent R^{4b}. In still another embodiment of formula I, it is substituted by R^{4b}.

In one embodiment, R⁴ is a 5-membered saturated carbocycle. According to one embodiment thereof, the carbocycle is unsubstituted, i.e. it does not carry any substituent R^{4b}. In still another embodiment of formula I, it is substituted by R^{4b}.

In one embodiment, R⁴ is a 6-membered saturated carbocycle. According to one embodiment thereof, the carbocycle is unsubstituted, i.e. it does not carry any substituent R^{4b}. In still another embodiment of formula I, it is substituted by R^{4b}.

In still another embodiment of formula I, R⁴ is five- or six-membered heteroaryl; wherein the heteroaryl contains independently one, two, three or four heteroatoms selected from N, O and S, and wherein the heterocycle are unsubstituted or substituted with substituents R^{4b} as defined below. According to one embodiment thereof, the heteroaryl in unsubtituted.

In still another embodiment of formula I, in the embodiment of R⁴ described above, the heteroaryl in substituted with substituents R^{4b} as defined below.

In still another embodiment of formula I, in the embodiment of R⁴ described above, the heteroaryl contains preferably one, two or three heteroatoms selected from N, O and S. More specifically, the heteroaryl contains one heteroatom selected from N, O and S. In particular the heteoaryl contains one or two, in particular one N.

In still another embodiment of formula I, in the embodiment of R⁴ described above, the 5-membered heteoaryl, in particular one N. According to one embodiment thereof, the heteroaryl is unsubtituted, i.e. it does not carry any substituyent R^{4b}.

In still another embodiment of formula I, in the embodiment of R⁴ described above, the 5-membered heteoaryl, in particular one N. According to one embodiment thereof, the heteroaryl is substituted by R^{4b}.ln still another embodiment of formula I, in the embodiment of R⁴ described above, the 5-membered heteoaryl contains one or two heteroatoms from N, O and S, in particular two N. According to one embodiment thereof, the heteroaryl is unsubtituted, i.e. it does not carry any substituyent R^{4b}.

In still another embodiment of formula I, in the embodiment of R⁴ described above, the 5-membered heteoaryl contains one or two heteroatoms from N, O and S, in particular two N. According to one embodiment thereof, the heteroaryl is substituted by R^{4b}.ln still another embodiment of formula I, in the embodiment of R⁴ described above, the 5-membered heteoaryl contains one or two heteroatoms from N, O and S in particu-lar one S. According to one embodiment thereof, the heteroaryl is unsubtituted, i.e. it does not carry any substituent.

In still another embodiment of formula I, in the embodiment of R⁴ described above, the 5-membered heteoaryl contains one or two heteroatoms from N, O and S in particular one S. According to one embodiment thereof, the heteroaryl is substituted by R^{4b}.

In still another embodiment of formula I, in the embodiment of R⁴ described above, the 5-membered heteoaryl contains one or two heteroatoms from N, O and S in particular one N and one S. According to one embodiment thereof, the heteroaryl is unsubtituted, i.e. it does not carry any substituyent R^{4b}.

In still another embodiment of formula I, in the embodiment of R⁴ described above, the 5-membered heteoaryl contains one or two heteroatoms from N, O and S in particular one N and one S. According to one embodiment thereof, the heteroaryl is substituted by R^{4b}.

In still another embodiment of formula I, in the embodiment of R⁴ described above, the 5-membered heteoaryl contains one or two heteroatoms from N, O and S in particular one N and one O. According to one embodiment thereof, the heteroaryl is unsubtituted, i.e. it does not carry any substituyent R^{4b}.

In still another embodiment of formula I, in the embodiment of R⁴ described above, the 5-membered heteoaryl contains one or two heteroatoms from N, O and S in particular one N and one O. According to one embodiment thereof, the heteroaryl is substituted by R^{4b}

In still another embodiment of formula I, in the embodiment of R⁴ described above, the 5-membered heteoaryl contains three heteroatoms from N, O and S, in particular three N. According to one embodiment thereof, the heteroaryl is unsubtituted, i.e. it does not carry any substituent R^{4b}.

In still another embodiment of formula I, in the embodiment of R⁴ described above, the 5-membered heteoaryl contains three heteroatoms from N, O and S, in particular three N. According to one embodiment thereof, the heteroaryl is substituted by R^{4b}

In still another embodiment of formula I, in the embodiment of R⁴ described above, the 5-membered heteoaryl contains three heteroatoms from N, O and S, in particular two N and one O. According to one embodiment thereof, the heteroaryl is unsubtituted, i.e. it does not carry any substituyent R^{4b}.

In still another embodiment of formula I, in the embodiment of R⁴ described above, the 5-membered heteoaryl contains three heteroatoms from N, O and S, in particular two N and one O. According to one embodiment thereof, the heteroaryl is substituted by R^{4b}.

In still another embodiment of formula I, in the embodiment of R⁴ described above, the 6-membered heteoaryl, in particular one N. According to one embodiment thereof, the heteroaryl is unsubtituted, i.e. it does not carry any substituyent R^{4b}.

In still another embodiment of formula I, in the embodiment of R⁴ described above, the 6-membered heteoaryl, in particular one N. According to one embodiment thereof, the heteroaryl is substituted by R^{4b}.

In still another embodiment of formula I, in the embodiment of R⁴ described above, the 6-membered heteoaryl contains one or two heteroatoms from N, O and S, in particular two N. According to one embodiment thereof, the heteroaryl is unsubtituted, i.e. it does not carry any substituent R^{4b}.

In still another embodiment of formula I, in the embodiment of R⁴ described above, the 6-membered heteoaryl contains one or two heteroatoms from N, O and S, in particular two N. According to one embodiment thereof, the heteroaryl is substituted by R^{4b}.

According to still another embodiment of formula I, R⁴ is a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl.

According to still another embodiment of formula I, R⁴ is a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

In still another embodiment of formula I, R⁴ is selected from C₁-C₆-alkyl, C₁-C₆-haloalkyl, phenyl-C₁-C₆-alkyl, halophenyl-C₁-C₆-alkyl, phenyl, halophenyl and three-, four-, five- or six-membered carbocycle, wherein the carbocycle is unsubstituted or substituted bysubstituents R^{4b} as defined below. According to one embodiment thereof, the carbocycle is unsubstituted. In a particular embodiment, R⁴ is selected from C₁-C₆-alkyl, C₁-C₆-haloalkyl, phenyl-CH₂, halo-phenyl-CH₂, phenyl, halophenyl and three-, four-, five- or six-membered carbocycle, wherein the carbocycle is unsubstituted or substituted bysubstituents R^{4b} as defined below.

In still another embodiment of formula I, R³, R⁴ together with the carbon atom to which they are bound form saturated, partially unsaturated three-, four-, five-, six-, seven-, eight-, nine-, or ten-membered carbocycle or heterocycle; wherein the heterocycle contains one, two, three or four heteroatoms selected from N, O and S, wherein the heteroatom N may carry one substituent selected from C₁-C₄-alkyl, C₁-C₄-haloalkyl and SO₂Ph, wherein Ph is unsubstituted phenyl or phenyl that is substituted by one, two or three substituents selected from C₁-C₄-alkyl, halogen, C₁-C₄-haloalkyl, C₁-C₄-alkoxy and C₁-C₄-haloalkoxy, and CN; and wherein the heteroatom S may be in the form of its oxide SO or SO₂, and wherein the carbocycle or heterocycle is unsubstituted or carries one, two, three or four substituents R³⁴ independently selected from halogen, OH, CN, NO₂, SH, NH₂, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₄-alkoxy-C₁-C₄-alkyl, phenyl and phenoxy, wherein the phenyl groups are unsubstituted or carry one, two, three, four or five substituents R^{34a} selected from the group consisting of halogen, OH, C₁-C₄-alkyl, C₁-C₄-haloalkyl, C₁-C₄-alkoxy, C₁-C₄-haloalkoxy and CN; and wherein in each case one or two CH₂ groups of the carbo- or heterocycle may be replaced by a group independently selected from C(═O) and C(═S); and

In one embodiment, R³ and R⁴ form a 3-membered saturated carbocycle. According to one embodiment thereof, the carbocycle is unsubstituted, i.e. it does not carry any substituent R^{4b}. In still another embodiment of formula I, it is substituted by R^{4b}.

In one embodiment, R³ and R⁴ form a 4-membered saturated carbocycle. According to one embodiment thereof, the carbocycle is unsubstituted, i.e. it does not carry any substituent R^{4b}. In still another embodiment of formula I, it is substituted by R^{4b}.

In one embodiment, R³ and R⁴ form a 5-membered saturated carbocycle. According to one embodiment thereof, the carbocycle is unsubstituted, i.e. it does not carry any substituent R^{4b}. In still another embodiment of formula I, it is substituted by R^{4b}.

In one embodiment, R³ and R⁴ form a 6-membered saturated carbocycle. According to one embodiment thereof, the carbocycle is unsubstituted, i.e. it does not carry any substituent R^{4b}. In still another embodiment of formula I, it is substituted by R^{4b}.

In one embodiment, R³ and R⁴ form a 7-membered saturated carbocycle. According to one embodiment thereof, the carbocycle is unsubstituted, i.e. it does not carry any substituent R^{4b}. In still another embodiment of formula I, it is substituted by R^{4b}.

In one embodiment, R³ and R⁴ form a 3-membered saturated heterocycle. According to one embodiment thereof, the heterocycle is unsubstituted, i.e. it does not carry any substituent R^{4b}.

In still another embodiment of formula I, it is substituted by R^{4b}.

According to one particular embodiment, R³ and R⁴ together form a 4-membered saturated heterocycle which contains 1 or 2 heteroatoms, in particular 1 heteroatom, from the group consisting of NH, NR^{N}, O, S, S(═O) and S(═O)₂, as ring members, wherein R^{N} is defined and preferably defined above. In one embodiment, the heterocycle contains one O as heteroatom. For example, the formed heterocycle is oxetane. According to one embodiment thereof, the heterocycle is unsubstituted, i.e. it does not carry any substituent R³⁴. According to a further embodiment, it carries one, two, three or four R³⁴.

According to a further particular embodiment, R³ and R⁴ together form a 5-membered saturated heterocycle which contains 1, 2 or 3, in particular 1 or 2, heteroatoms from the group consisting of NH, NR^{N}, O, S, S(═O) and S(═O)₂, as ring members, wherein R^{N} is as defined and preferably defined above. According to one embodiment thereof, the heterocycle is unsubstituted, i.e. it does not carry any substituent R³⁴. According to a further embodiment, it carries one, two, three or four R³⁴.

According to a further particular embodiment, R³ and R⁴ together form a 6-membered saturated heterocycle which contains 1, 2 or 3, in particular 1 or 2, heteroatoms from the group consisting of NH, NR^{N}, O, S, S(═O) and S(═O)₂, as ring members, wherein R^{N} is as defined and preferably defined below. According to one embodiment thereof, the heterocycle is unsubstituted, i.e. it does not carry any substituent R³⁴.

According to a further particular embodiment, R³ and R⁴ together form a 7-membered saturated heterocycle which contains 1, 2 or 3, in particular 1 or 2, heteroatoms from the group consisting of NH, NR^{N}, O, S, S(═O) and S(═O)₂, as ring members, wherein R^{N} is as defined and preferably defined below. According to one embodiment thereof, the heterocycle is unsubstituted, i.e. it does not carry any substituent R³⁴.

According to one further embodiment R³ together with R⁴ and with the carbon atom to which they are bound form a saturated three-, four-, five-, six-, seven-, eight-, nine-, or ten-membered, in particular three-, four-, five- or six-membered carbocycle, more specifically five- or six-membered carbocycle, that is unsubstituted or carries one, two, three or four substituents R³⁴ as defined below. According to one embodiment thereof, R³ and R⁴ form a cyclopropyl, that is unsubstituted or carries one, two, three or four substituents R³⁴ as defined below. According to a further embodiment thereof, R³ and R⁴ form a cyclobutyl, that is unsubstituted or carries one, two, three or four substituents R³⁴ as defined below. According to still a further embodiment thereof, R³ and R⁴ form a cyclopentyl, that is unsubstituted or carries one, two, three or four substituents R³⁴ as defined below. According to still a further embodiment thereof, R³ and R⁴ form a cyclohexyl, that is unsubstituted or carries one, two, three or four substituents R³⁴ as defined below. According to still a further embodiment thereof, R³ and R⁴ form a cycloheptyl, that is unsubstituted or carries one, two, three or four substituents R³⁴ as defined below.

R³⁴ are the possible substituents for the carbo- or heterocycle formed by R³ and R⁴ and are independently selected from halogen, OH, CN, NO₂, SH, NH₂, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₄-alkoxy-C₁-C₄-alkyl, phenyl and phenoxy, wherein the phenyl groups are unsubstituted or carry one, two, three, four or five substituents R³⁴a selected from the group consisting of halogen, OH, C₁-C₄-alkyl, C₁-C₄-haloalkyl, C₁-C₄-alkoxy and C₁-C₄-haloalkoxy; and wherein in each case one or two CH₂ groups of the carbo- or heterocycle may be replaced by a group independently selected from C(═O) and C(═S).

In one preferred embodiment, R³⁴ is in each case independently selected from halogen, OH, CN, SH, C₁-C₆-alkyl, C₁-C₆-haloalkyl, C₁-C₆-alkoxy, C₁-C₆-haloalkoxy and C₁-C₆-alkylthio. In one further preferred embodiment, R³⁴ is in each case independently selected from halogen, C₁-C₆-alkyl and C₁-C₆-haloalkyl. In one further particular embodiment, R³⁴ is in each case independently selected from C₁-C₆-alkyl, such as methyl and ethyl.

R^{N} is the substituent of the heteroatom NR^{N} that is contained in the heterocycle formed by R³ and R⁴ in some of the inventive compounds. R^{N} is selected from C₁-C₄-alkyl, C₁-C₄-haloalkyl and SO₂Ph, wherein Ph is unsubstituted phenyl or phenyl that is substituted by one, two or three substituents selected from C₁-C₄-alkyl. In one preferred embodiment, R^{N} is in each case independently selected from C₁-C₂-alkyl, C₁-C₂-haloalkyl and SO₂Ph, wherein Ph is unsubstituted phenyl or phenyl that is substituted by one methyl substituents. In one particular embodiment, R^{N} is in each case independently selected from C₁-C₂-alkyl, more particularly methyl. In one particular embodiment, R^{N} is in each case independently selected from SO₂Ph, wherein Ph is unsubstituted phenyl or phenyl that is substituted by one methyl.

Particularly preferred embodiments of R⁴ according to the invention are in Table P4 below, wherein each line of lines P4-1 to P4-127 corresponds to one particular embodiment of the invention, wherein P4-1 to P4-127 are also in any combination with one another a preferred embodiment of the present invention. The connection point to the carbon atom, to which R⁴ is bound is marked with “#” in the drawings.

TABLE P4
(py = pyridyl):

| No. | R⁴ |
|---|---|
| P4-1 | CH₃ |
| P4-2 | C₂H₅ |
| P4-3 | CH₂CH₂CH₃ |
| P4-4 | CH(CH₃)₂ |
| P4-5 | CH₂CH₂CH₂CH₃ |
| P4-6 | CH₂CH(CH₃)₂ |
| P4-7 | C(CH₃)₃ |
| P4-8 | CH₂CH₂CH₂CH₂CH₃ |
| P4-9 | CH₂CH₂CH(CH₃)₂ |
| P4-10 | CF₃ |
| P4-11 | CHF₂ |
| P4-12 | CHCl₂ |
| P4-13 | CH₂F |
| P4-14 | CH₂Cl |
| P4-15 | CH₂CF₃ |
| P4-16 | CH₂CCl₃ |
| P4-17 | CF₂CHF₂ |
| P4-18 | C₆H₅ |
| P4-19 | 4-Cl—C₆H₄ |
| P4-20 | 4-F—C₆H₄ |
| P4-21 | CH₂—C₆H₅ |
| P4-22 | 3-py |
| P4-23 | 2-py |
| P4-24 | 4-py |
| P4-25 | 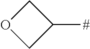 |
| P4-26 | 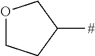 |
| P4-27 | 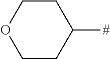 |
| P4-28 | 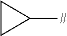 |
| P4-29 | 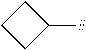 |
| P4-30 | 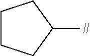 |
| P4-31 | 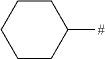 |
| P4-32 | 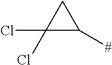 |
| P4-33 | CH₂OCF₃ |
| P4-34 | CN |
| P4-35 | CH₂OMe |
| P4-36 | CH₂OCH₂F |
| P4-37 | CH₂OCHF₂ |
| P4-38 | CHO |
| P4-39 | COMe |
| P4-40 | CO₂Me |
| P4-41 | CH₂CN |
| P4-42 | CH₂SMe |
| P4-43 | CH₂CHO |
| P4-44 | CH₂COMe |
| P4-45 | CH₂CO₂Me |
| P4-46 | CH₂NMe₂ |
| P4-47 | 4-OMe—C₆H₄ |
| P4-48 | 3-OMe—C₆H₄ |
| P4-49 | 2-OMe—C₆H₄ |
| P4-50 | 3-F—C₆H₄ |
| P4-51 | 2-F—C₆H₄ |
| P4-52 | 2,4-F₂—C₆H₃ |
| P4-53 | 3-Cl—C₆H₄ |
| P4-54 | 2-Cl—C₆H₄ |
| P4-55 | 2,4-Cl₂—C₆H₃ |
| P4-56 | CH₂—C₆H₄-4-F |
| P4-57 | CH₂—C₆H₄-4-Cl |
| P4-58 | CH₂—C₆H₃-2,4-Cl₂ |
| P4-59 | 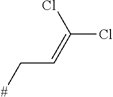 |
| P4-60 | 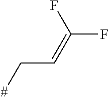 |
| P4-61 | 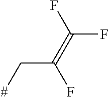 |
| P4-62 | 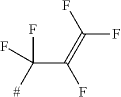 |
| P4-63 | 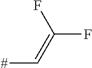 |
| P4-64 | 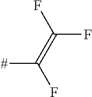 |
| P4-65 | 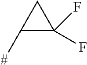 |
| P4-66 | 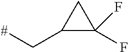 |
| P4-67 | 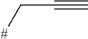 |
| P4-68 | 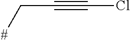 |
| P4-69 | 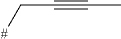 |
| P4-70 | 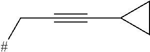 |
| P4-71 | 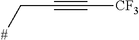 |
| P4-72 | 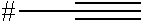 |
| P4-73 | 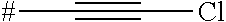 |
| P4-74 | 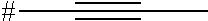 |
| P4-75 | 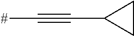 |
| P4-76 | 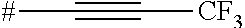 |
| P4-77 | 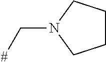 |
| P4-78 | 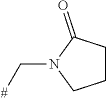 |
| P4-79 | 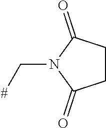 |
| P4-80 | 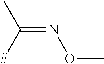 |
| P4-81 | 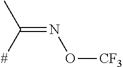 |
| P4-82 | 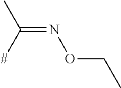 |
| P4-83 | 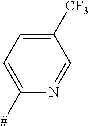 |
| P4-84 | 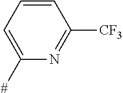 |
| P4-85 | 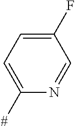 |
| P4-86 | 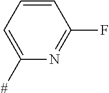 |
| P4-87 | 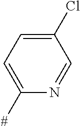 |
| P4-88 | 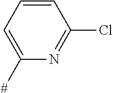 |
| P4-89 | 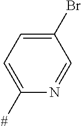 |
| P4-90 | 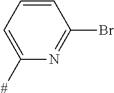 |
| P4-91 | 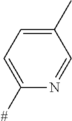 |
| P4-92 | 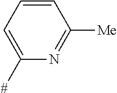 |
| P4-93 | 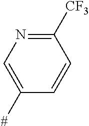 |
| P4-94 | 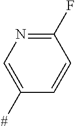 |
| P4-95 | 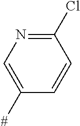 |
| P4-96 | 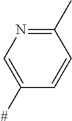 |
| P4-97 | 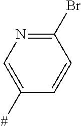 |
| P4-98 | 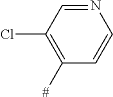 |
| P4-99 | 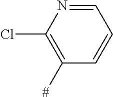 |
| P4-100 | 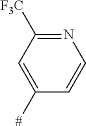 |
| P4-101 | 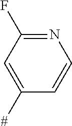 |
| P4-102 | 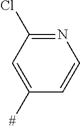 |
| P4-103 | 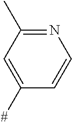 |
| P4-104 | 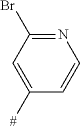 |
| P4-105 | 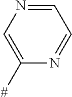 |
| P4-106 | 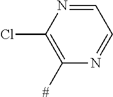 |
| P4-107 | 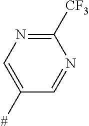 |
| P4-108 | 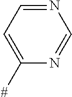 |
| P4-109 | 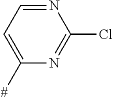 |
| P4-110 | 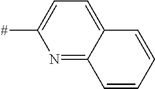 |
| P4-111 | 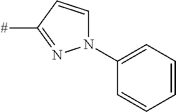 |
| P4-112 | 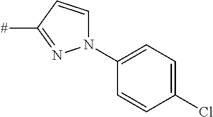 |
| P4-113 | 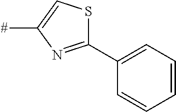 |
| P4-114 | 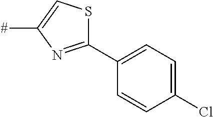 |
| P4-115 | 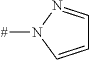 |
| P4-116 | 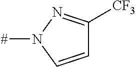 |
| P4-117 | 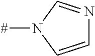 |
| P4-118 | 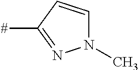 |
| P4-119 | 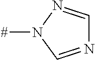 |
| P4-120 | 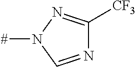 |
| P4-121 | 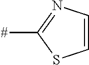 |
| P4-122 | 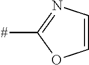 |
| P4-123 | 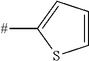 |
| P4-124 | 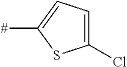 |
| P4-125 | 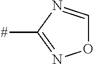 |
| P4-126 | 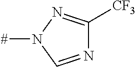 |
| P4-127 | 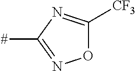 |

Particularly preferred embodiments of combinations of R³ and R⁴ according to the invention are in Table P34 below, wherein each line of lines P34-1 to P34-487 corresponds to one particular embodiment of the invention, wherein P34-1 to P34-487 are also in any combination with one another a preferred embodiment of the present invention. The carbon atom, to which R³ and R⁴ are bound is marked with * in the drawings. “Ts” in the drawings stands for the tosylgroup SO₂-(p-CH₃)phenyl.

TABLE P34
(py = pyridyl):

| No. | R³ | R⁴ |
|---|---|---|
| P34-1 | CH₃ | CH₃ |
| P34-2 | CH₃ | C₂H₅ |
| P34-3 | CH₃ | CH₂CH₂CH₃ |
| P34-4 | CH₃ | CH(CH₃)₂ |
| P34-5 | CH₃ | CH₂CH₂CH₂CH₃ |
| P34-6 | CH₃ | CH₂CH(CH₃)₂ |
| P34-7 | CH₃ | C(CH₃)₃ |
| P34-8 | CH₃ | CH₂CH₂CH₂CH₂CH₃ |
| P34-9 | CH₃ | CH₂CH₂CH(CH₃)₂ |
| P34-10 | CH₃ | CF₃ |
| P34-11 | CH₃ | CHF₂ |
| P34-12 | CH₃ | CHCl₂ |
| P34-13 | CH₃ | CH₂F |
| P34-14 | CH₃ | CH₂Cl |
| P34-15 | CH₃ | CH₂CF₃ |
| P34-16 | CH₃ | CH₂CCl₃ |
| P34-17 | CH₃ | CF₂CHF₂ |
| P34-18 | CH₃ | C₆H₅ |
| P34-19 | CH₃ | 4-Cl—C₆H₄ |
| P34-20 | CH₃ | 4-F—C₆H₄ |
| P34-21 | CH₃ | CH₂—C₆H₅ |
| P34-22 | CH₃ | 3-py |
| P34-23 | CH₃ | 2-py |
| P34-24 | CH₃ | 4-py |
| P34-25 | CH₃ | 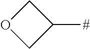 |
| P34-26 | CH₃ | 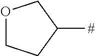 |
| P34-27 | CH₃ | 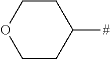 |
| P34-28 | CH₃ | 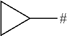 |
| P34-29 | CH₃ | 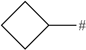 |
| P34-30 | CH₃ | 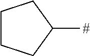 |
| P34-31 | CH₃ | 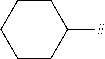 |
| P34-32 | CH₃ | 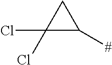 |
| P34-33 | CH₃ | CH₂OCF₃ |
| P34-34 | CH₃ | CN |
| P34-35 | CH₃ | CH₂OMe |
| P34-36 | CH₃ | CH₂OCH₂F |
| P34-37 | CH₃ | CH₂OCHF₂ |
| P34-38 | CH₃ | CHO |
| P34-39 | CH₃ | COMe |
| P34-40 | CH₃ | CO₂Me |
| P34-41 | CH₃ | CH₂CN |
| P34-42 | CH₃ | CH₂SMe |
| P34-43 | CH₃ | CH₂CHO |
| P34-44 | CH₃ | CH₂COMe |
| P34-45 | CH₃ | CH₂CO₂Me |
| P34-46 | CH₃ | CH₂NMe₂ |
| P34-47 | CH₃ | 4-OMe—C₆H₄ |
| P34-48 | CH₃ | 3-OMe—C₆H₄ |
| P34-49 | CH₃ | 2-OMe—C₆H₄ |
| P34-50 | CH₃ | 3-F—C₆H₄ |
| P34-51 | CH₃ | 2-F—C₆H₄ |
| P34-52 | CH₃ | 2,4-F₂—C₆H₃ |
| P34-53 | CH₃ | 3-Cl—C₆H₄ |
| P34-54 | CH₃ | 2-Cl—C₆H₄ |
| P34-55 | CH₃ | 2,4-Cl₂—C₆H₃ |
| P34-56 | CH₃ | CH₂—C₆H₄-4-F |
| P34-57 | CH₃ | CH₂—C₆H₄-4-Cl |
| P34-58 | CH₃ | CH₂—C₆H₃-2,4-Cl₂ |
| P34-59 | CH₃ | 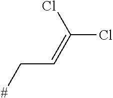 |
| P34-60 | CH₃ | 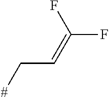 |
| P34-61 | CH₃ | 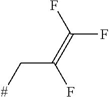 |
| P34-62 | CH₃ | 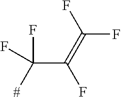 |
| P34-63 | CH₃ | 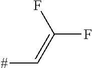 |
| P34-64 | CH₃ | 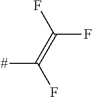 |
| P34-65 | CH₃ | 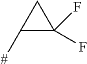 |
| P34-66 | CH₃ | 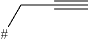 |
| P34-67 | CH₃ | 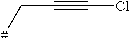 |
| P34-68 | CH₃ | 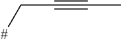 |
| P34-69 | CH₃ | 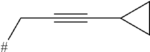 |
| P34-70 | CH₃ | 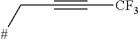 |
| P34-71 | CH₃ | 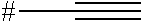 |
| P34-72 | CH₃ | 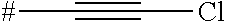 |
| P34-73 | CH₃ | 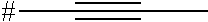 |
| P34-74 | CH₃ | 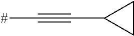 |
| P34-75 | CH₃ | 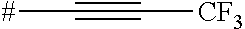 |
| P34-76 | CH₃ | 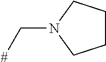 |
| P34-77 | CH₃ | 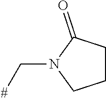 |
| P34-78 | CH₃ | 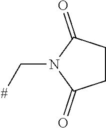 |
| P34-79 | CH₃ | 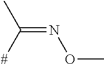 |
| P34-80 | CH₃ | 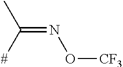 |
| P34-81 | CH₃ | 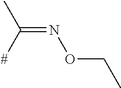 |
| P34-82 | CH₃ | 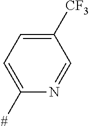 |
| P34-83 | CH₃ | 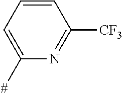 |
| P34-84 | CH₃ | 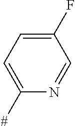 |
| P34-85 | CH₃ | 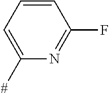 |
| P34-86 | CH₃ | 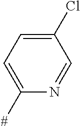 |
| P34-87 | CH₃ | 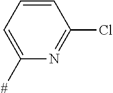 |
| P34-88 | CH₃ | 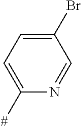 |
| P34-89 | CH₃ | 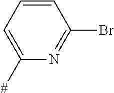 |
| P34-90 | CH₃ | 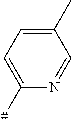 |
| P34-91 | CH₃ | 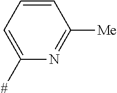 |
| P34-92 | CH₃ | 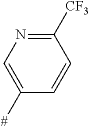 |
| P34-93 | CH₃ | 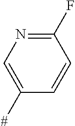 |
| P34-94 | CH₃ | 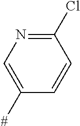 |
| P34-95 | CH₃ | 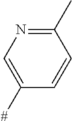 |
| P34-96 | CH₃ | 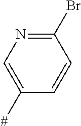 |
| P34-97 | CH₃ | 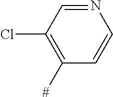 |
| P34-98 | CH₃ | 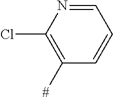 |
| P34-99 | CH₃ | 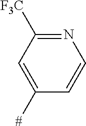 |
| P34-100 | CH₃ | 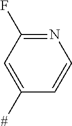 |
| P34-101 | CH₃ | 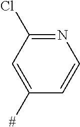 |
| P34-102 | CH₃ | 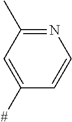 |
| P34-103 | CH₃ | 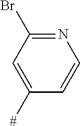 |
| P34-104 | CH₃ | 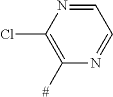 |
| P34-105 | CH₃ | 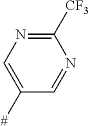 |
| P34-106 | CH₃ | 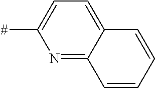 |
| P34-107 | CH₃ | 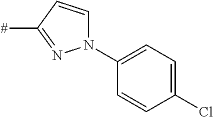 |
| P34-108 | CH₃ | 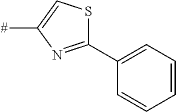 |
| P34-109 | CH₃ | 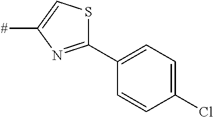 |
| P34-110 | CH₃ | 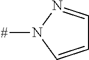 |
| P34-111 | CH₃ | 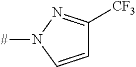 |
| P34-112 | CH₃ | 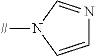 |
| P34-113 | CH₃ | 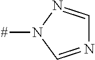 |
| P34-114 | CH₃ | 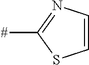 |
| P34-115 | CH₃ | 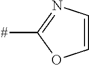 |
| P34-116 | CH₃ | 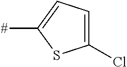 |
| P34-117 | CH₃ | 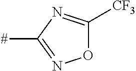 |
| P34-118 | CH₂F | CH₃ |
| P34-119 | CH₂F | C₂H₅ |
| P34-120 | CH₂F | CH₂CH₂CH₃ |
| P34-121 | CH₂F | CH(CH₃)₂ |
| P34-122 | CH₂F | CH₂CH₂CH₂CH₃ |
| P34-123 | CH₂F | CH₂CH(CH₃)₂ |
| P34-124 | CH₂F | C(CH₃)₃ |
| P34-125 | CH₂F | CH₂CH₂CH₂CH₂CH₃ |
| P34-126 | CH₂F | CH₂CH₂CH(CH₃)₂ |
| P34-127 | CH₂F | CF₃ |
| P34-128 | CH₂F | CHF₂ |
| P34-129 | CH₂F | CHCl₂ |
| P34-130 | CH₂F | CH₂F |
| P34-131 | CH₂F | CH₂Cl |
| P34-132 | CH₂F | CH₂CF₃ |
| P34-133 | CH₂F | CH₂CCl₃ |
| P34-134 | CH₂F | CF₂CHF₂ |
| P34-135 | CH₂F | C₆H₅ |
| P34-136 | CH₂F | 4-Cl—C₆H₄ |
| P34-137 | CH₂F | 4-F—C₆H₄ |
| P34-138 | CH₂F | CH₂—C₆H₅ |
| P34-139 | CH₂F | 3-py |
| P34-140 | CH₂F | 2-py |
| P34-141 | CH₂F | 4-py |
| P34-142 | CH₂F | 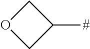 |
| P34-143 | CH₂F | 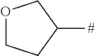 |
| P34-144 | CH₂F | 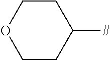 |
| P34-145 | CH₂F | 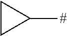 |
| P34-146 | CH₂F | 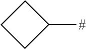 |
| P34-147 | CH₂F | 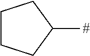 |
| P34-148 | CH₂F | 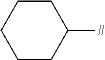 |
| P34-149 | CH₂F | 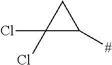 |
| P34-150 | CH₂F | CH₂OCF₃ |
| P34-151 | CH₂F | CN |
| P34-152 | CH₂F | CH₂OMe |
| P34-153 | CH₂F | CH₂OCH₂F |
| P34-154 | CH₂F | CH₂OCHF₂ |
| P34-155 | CH₂F | CHO |
| P34-156 | CH₂F | COMe |
| P34-157 | CH₂F | CO₂Me |
| P34-158 | CH₂F | CH₂CN |
| P34-159 | CH₂F | CH₂SMe |
| P34-160 | CH₂F | CH₂CHO |
| P34-161 | CH₂F | CH₂COMe |
| P34-162 | CH₂F | CH₂CO₂Me |
| P34-163 | CH₂F | CH₂NMe₂ |
| P34-164 | CH₂F | 4-OMe—C₆H₄ |
| P34-165 | CH₂F | 3-OMe—C₆H₄ |
| P34-166 | CH₂F | 2-OMe—C₆H₄ |
| P34-167 | CH₂F | 3-F—C₆H₄ |
| P34-168 | CH₂F | 2-F—C₆H₄ |
| P34-169 | CH₂F | 2,4-F₂—C₆H₃ |
| P34-170 | CH₂F | 3-Cl—C₆H₄ |
| P34-171 | CH₂F | 2-Cl—C₆H₄ |
| P34-172 | CH₂F | 2,4-Cl₂—C₆H₃ |
| P34-173 | CH₂F | CH₂—C₆H₄-4-F |
| P34-174 | CH₂F | CH₂—C₆H₄-4-Cl |
| P34-175 | CH₂F | CH₂—C₆H₃-2,4-Cl₂ |
| P34-176 | CH₂F | 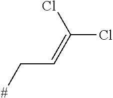 |
| P34-177 | CH₂F | 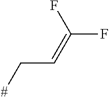 |
| P34-178 | CH₂F | 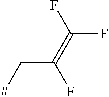 |
| P34-179 | CH₂F | 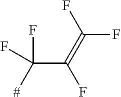 |
| P34-180 | CH₂F | 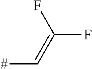 |
| P34-181 | CH₂F | 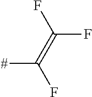 |
| P34-182 | CH₂F | 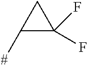 |
| P34-183 | CH₂F | 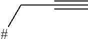 |
| P34-184 | CH₂F | 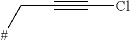 |
| P34-185 | CH₂F | 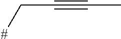 |
| P34-186 | CH₂F | 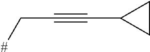 |
| P34-187 | CH₂F | 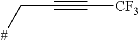 |
| P34-188 | CH₂F | 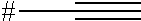 |
| P34-189 | CH₂F | 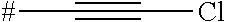 |
| P34-190 | CH₂F | 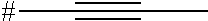 |
| P34-191 | CH₂F | 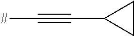 |
| P34-192 | CH₂F | 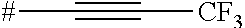 |
| P34-193 | CH₂F | 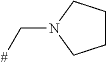 |
| P34-194 | CH₂F | 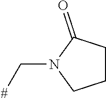 |
| P34-195 | CH₂F | 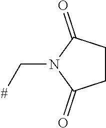 |
| P34-196 | CH₂F | 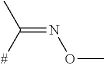 |
| P34-197 | CH₂F | 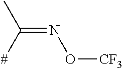 |
| P34-198 | CH₂F | 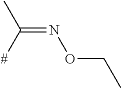 |
| P34-199 | CH₂F | 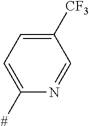 |
| P34-200 | CH₂F | 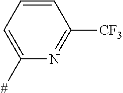 |
| P34-201 | CH₂F | 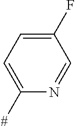 |
| P34-202 | CH₂F | 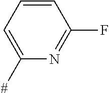 |
| P34-203 | CH₂F | 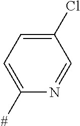 |
| P34-204 | CH₂F | 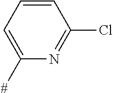 |
| P34-205 | CH₂F | 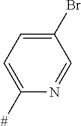 |
| P34-206 | CH₂F | 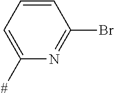 |
| P34-207 | CH₂F | 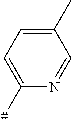 |
| P34-208 | CH₂F | 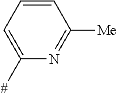 |
| P34-209 | CH₂F | 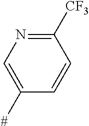 |
| P34-210 | CH₂F | 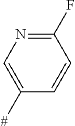 |
| P34-211 | CH₂F | 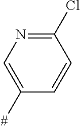 |
| P34-212 | CH₂F | 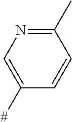 |
| P34-213 | CH₂F | 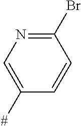 |
| P34-214 | CH₂F | 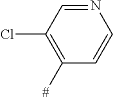 |
| P34-215 | CH₂F | 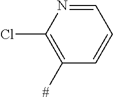 |
| P34-216 | CH₂F | 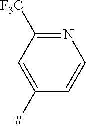 |
| P34-217 | CH₂F | 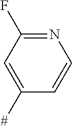 |
| P34-218 | CH₂F | 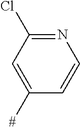 |
| P34-219 | CH₂F | 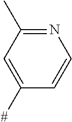 |
| P34-220 | CH₂F | 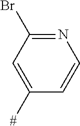 |
| P34-221 | CH₂F | 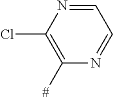 |
| P34-222 | CH₂F | 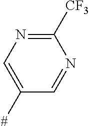 |
| P34-223 | CH₂F | 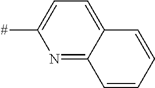 |
| P34-224 | CH₂F | 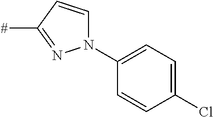 |
| P34-225 | CH₂F | 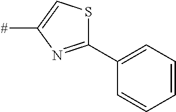 |
| P34-226 | CH₂F | 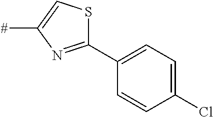 |
| P34-227 | CH₂F | 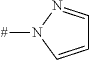 |
| P34-228 | CH₂F | 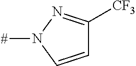 |
| P34-229 | CH₂F | 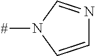 |
| P34-230 | CH₂F | 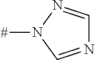 |
| P34-231 | CH₂F | 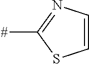 |
| P34-232 | CH₂F | 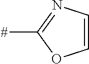 |
| P34-233 | CH₂F | 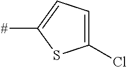 |
| P34-234 | CH₂F | 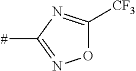 |
| P34-235 | CHF₂ | CH₃ |
| P34-236 | CHF₂ | C₂H₅ |
| P34-237 | CHF₂ | CH₂CH₂CH₃ |
| P34-238 | CHF₂ | CH(CH₃)₂ |
| P34-238 | CHF₂ | CH₂CH₂CH₂CH₃ |
| P34-240 | CHF₂ | CH₂CH(CH₃)₂ |
| P34-241 | CHF₂ | C(CH₃)₃ |
| P34-242 | CHF₂ | CH₂CH₂CH₂CH₂CH₃ |
| P34-243 | CHF₂ | CH₂CH₂CH(CH₃)₂ |
| P34-244 | CHF₂ | CF₃ |
| P34-245 | CHF₂ | CHF₂ |
| P34-246 | CHF₂ | CHCl₂ |
| P34-247 | CHF₂ | CH₂F |
| P34-248 | CHF₂ | CH₂Cl |
| P34-249 | CHF₂ | CH₂CF₃ |
| P34-250 | CHF₂ | CH₂CCl₃ |
| P34-251 | CHF₂ | CF₂CHF₂ |
| P34-252 | CHF₂ | C₆H₅ |
| P34-253 | CHF₂ | 4-Cl—C₆H₄ |
| P34-254 | CHF₂ | 4-F—C₆H₄ |
| P34-255 | CHF₂ | CH₂—C₆H₅ |
| P34-256 | CHF₂ | 3-py |
| P34-257 | CHF₂ | 2-py |
| P34-258 | CHF₂ | 4-py |
| P34-259 | CHF₂ | 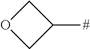 |
| P34-260 | CHF₂ | 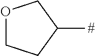 |
| P34-261 | CHF₂ | 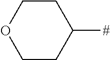 |
| P34-262 | CHF₂ | 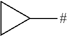 |
| P34-263 | CHF₂ | 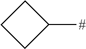 |
| P34-264 | CHF₂ | 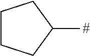 |
| P34-265 | CHF₂ | 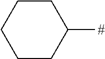 |
| P34-266 | CHF₂ | 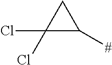 |
| P34-267 | CHF₂ | CH₂OCF₃ |
| P34-268 | CHF₂ | CN |
| P34-269 | CHF₂ | CH₂OMe |
| P34-270 | CHF₂ | CH₂OCH₂F |
| P34-271 | CHF₂ | CH₂OCHF₂ |
| P34-272 | CHF₂ | CHO |
| P34-273 | CHF₂ | COMe |
| P34-274 | CHF₂ | CO₂Me |
| P34-275 | CHF₂ | CH₂CN |
| P34-276 | CHF₂ | CH₂SMe |
| P34-277 | CHF₂ | CH₂CHO |
| P34-278 | CHF₂ | CH₂COMe |
| P34-279 | CHF₂ | CH₂CO₂Me |
| P34-280 | CHF₂ | CH₂NMe₂ |
| P34-281 | CHF₂ | 4-OMe—C₆H₄ |
| P34-282 | CHF₂ | 3-OMe—C₆H₄ |
| P34-283 | CHF₂ | 2-OMe—C₆H₄ |
| P34-284 | CHF₂ | 3-F—C₆H₄ |
| P34-285 | CHF₂ | 2-F—C₆H₄ |
| P34-286 | CHF₂ | 2,4-F₂—C₆H₃ |
| P34-287 | CHF₂ | 3-Cl—C₆H₄ |
| P34-288 | CHF₂ | 2-Cl—C₆H₄ |
| P34-289 | CHF₂ | 2,4-Cl₂—C₆H₃ |
| P34-290 | CHF₂ | CH₂—C₆H₄-4-F |
| P34-291 | CHF₂ | CH₂—C₆H₄-4-Cl |
| P34-292 | CHF₂ | CH₂—C₆H₃-2,4-Cl₂ |
| P34-293 | CHF₂ | 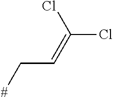 |
| P34-294 | CHF₂ | 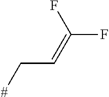 |
| P34-295 | CHF₂ | 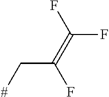 |
| P34-296 | CHF₂ | 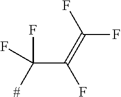 |
| P34-297 | CHF₂ | 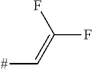 |
| P34-298 | CHF₂ | 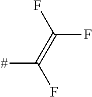 |
| P34-299 | CHF₂ | 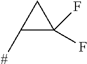 |
| P34-300 | CHF₂ | 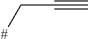 |
| P34-301 | CHF₂ | 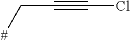 |
| P34-302 | CHF₂ | 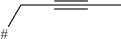 |
| P34-303 | CHF₂ | 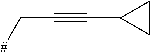 |
| P34-304 | CHF₂ | 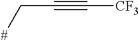 |
| P34-305 | CHF₂ | 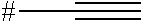 |
| P34-306 | CHF₂ | 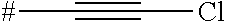 |
| P34-307 | CHF₂ | 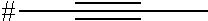 |
| P34-308 | CHF₂ | 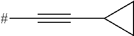 |
| P34-309 | CHF₂ | 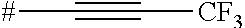 |
| P34-310 | CHF₂ | 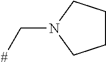 |
| P34-311 | CHF₂ | 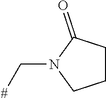 |
| P34-312 | CHF₂ | 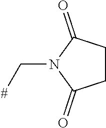 |
| P34-313 | CHF₂ | 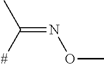 |
| P34-314 | CHF₂ | 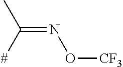 |
| P34-315 | CHF₂ | 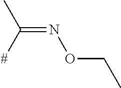 |
| P34-316 | CHF₂ | 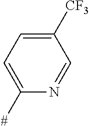 |
| P34-317 | CHF₂ | 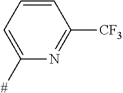 |
| P34-318 | CHF₂ | 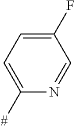 |
| P34-319 | CHF₂ | 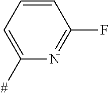 |
| P34-320 | CHF₂ | 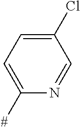 |
| P34-321 | CHF₂ | 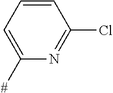 |
| P34-322 | CHF₂ | 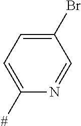 |
| P34-323 | CHF₂ | 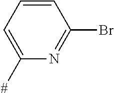 |
| P34-324 | CHF₂ | 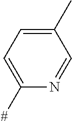 |
| P34-325 | CHF₂ | 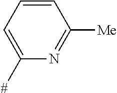 |
| P34-326 | CHF₂ | 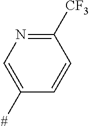 |
| P34-327 | CHF₂ | 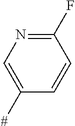 |
| P34-328 | CHF₂ | 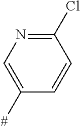 |
| P34-329 | CHF₂ | 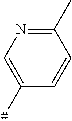 |
| P34-330 | CHF₂ | 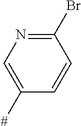 |
| P34-331 | CHF₂ | 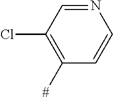 |
| P34-332 | CHF₂ | 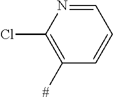 |
| P34-333 | CHF₂ | 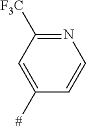 |
| P34-334 | CHF₂ | 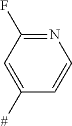 |
| P34-335 | CHF₂ | 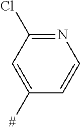 |
| P34-336 | CHF₂ | 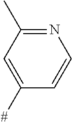 |
| P34-337 | CHF₂ | 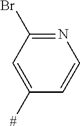 |
| P34-338 | CHF₂ | 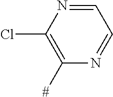 |
| P34-339 | CHF₂ | 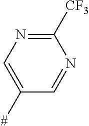 |
| P34-340 | CHF₂ | 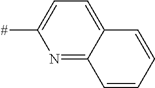 |
| P34-341 | CHF₂ | 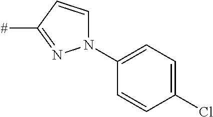 |
| P34-342 | CHF₂ | 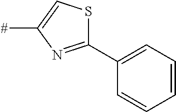 |
| P34-343 | CHF₂ | 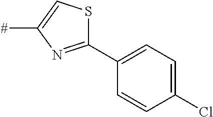 |
| P34-344 | CHF₂ | 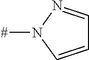 |
| P34-345 | CHF₂ | 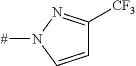 |
| P34-346 | CHF₂ | 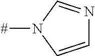 |
| P34-347 | CHF₂ | 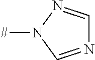 |
| P34-348 | CHF₂ | 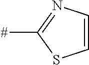 |
| P34-349 | CHF₂ | 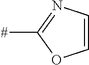 |
| P34-350 | CHF₂ | 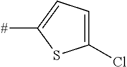 |
| P34-351 | CHF₂ | 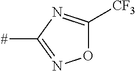 |
| P34-352 | CF₃ | CH₃ |
| P34-353 | CF₃ | C₂H₅ |
| P34-354 | CF₃ | CH₂CH₂CH₃ |
| P34-355 | CF₃ | CH(CH₃)₂ |
| P34-356 | CF₃ | CH₂CH₂CH₂CH₃ |
| P34-357 | CF₃ | CH₂CH(CH₃)₂ |
| P34-358 | CF₃ | C(CH₃)₃ |
| P34-359 | CF₃ | CH₂CH₂CH₂CH₂CH₃ |
| P34-360 | CF₃ | CH₂CH₂CH(CH₃)₂ |
| P34-361 | CF₃ | CF₃ |
| P34-362 | CF₃ | CHF₂ |
| P34-363 | CF₃ | CHCl₂ |
| P34-364 | CF₃ | CH₂F |
| P34-365 | CF₃ | CH₂Cl |
| P34-366 | CF₃ | CH₂CF₃ |
| P34-367 | CF₃ | CH₂CCl₃ |
| P34-368 | CF₃ | CF₂CHF₂ |
| P34-369 | CF₃ | C₆H₅ |
| P34-370 | CF₃ | 4-Cl—C₆H₄ |
| P34-371 | CF₃ | 4-F—C₆H₄ |
| P34-372 | CF₃ | CH₂—C₆H₅ |
| P34-373 | CF₃ | 3-py |
| P34-374 | CF₃ | 2-py |
| P34-375 | CF₃ | 4-py |
| P34-376 | CF₃ | 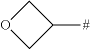 |
| P34-377 | CF₃ | 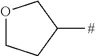 |
| P34-378 | CF₃ | 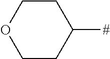 |
| P34-379 | CF₃ | 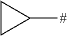 |
| P34-380 | CF₃ | 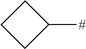 |
| P34-381 | CF₃ | 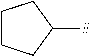 |
| P34-382 | CF₃ | 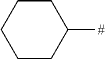 |
| P34-383 | CF₃ | 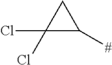 |
| P34-384 | CF₃ | CH₂OCF₃ |
| P34-385 | CF₃ | CN |
| P34-386 | CF₃ | CH₂OMe |
| P34-387 | CF₃ | CH₂OCH₂F |
| P34-388 | CF₃ | CH₂OCHF₂ |
| P34-389 | CF₃ | CHO |
| P34-390 | CF₃ | COMe |
| P34-391 | CF₃ | CO₂Me |
| P34-392 | CF₃ | CH₂CN |
| P34-393 | CF₃ | CH₂SMe |
| P34-394 | CF₃ | CH₂CHO |
| P34-395 | CF₃ | CH₂COMe |
| P34-396 | CF₃ | CH₂CO₂Me |
| P34-397 | CF₃ | CH₂NMe₂ |
| P34-398 | CF₃ | 4-OMe—C₆H₄ |
| P34-399 | CF₃ | 3-OMe—C₆H₄ |
| P34-400 | CF₃ | 2-OMe—C₆H₄ |
| P34-401 | CF₃ | 3-F—C₆H₄ |
| P34-402 | CF₃ | 2-F—C₆H₄ |
| P34-403 | CF₃ | 2,4-F₂—C₆H₃ |
| P34-404 | CF₃ | 3-Cl—C₆H₄ |
| P34-405 | CF₃ | 2-Cl—C₆H₄ |
| P34-406 | CF₃ | 2,4-Cl₂—C₆H₃ |
| P34-407 | CF₃ | CH₂—C₆H₄-4-F |
| P34-408 | CF₃ | CH₂—C₆H₄-4-Cl |
| P34-409 | CF₃ | CH₂—C₆H₃-2,4-Cl₂ |
| P34-410 | CF₃ | 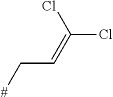 |
| P34-411 | CF₃ | 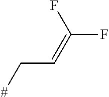 |
| P34-412 | CF₃ | 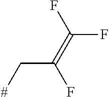 |
| P34-413 | CF₃ | 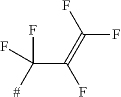 |
| P34-414 | CF₃ | 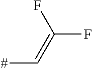 |
| P34-415 | CF₃ | 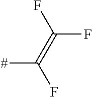 |
| P34-416 | CF₃ | 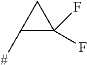 |
| P34-417 | CF₃ | 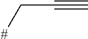 |
| P34-418 | CF₃ | 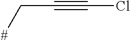 |
| P34-419 | CF₃ | 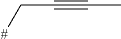 |
| P34-420 | CF₃ | 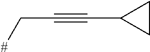 |
| P34-421 | CF₃ | 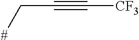 |
| P34-422 | CF₃ | 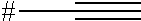 |
| P34-423 | CF₃ | 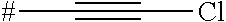 |
| P34-424 | CF₃ | 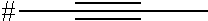 |
| P34-425 | CF₃ | 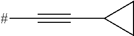 |
| P34-426 | CF₃ | 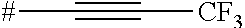 |
| P34-427 | CF₃ | 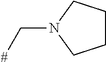 |
| P34-428 | CF₃ | 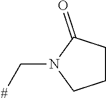 |
| P34-429 | CF₃ | 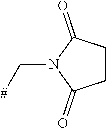 |
| P34-430 | CF₃ | 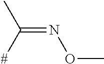 |
| P34-431 | CF₃ | 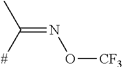 |
| P34-432 | CF₃ | 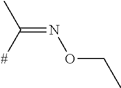 |
| P34-433 | CF₃ | 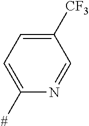 |
| P34-434 | CF₃ | 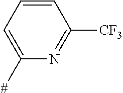 |
| P34-435 | CF₃ | 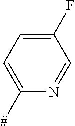 |
| P34-436 | CF₃ | 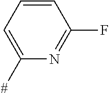 |
| P34-437 | CF₃ | 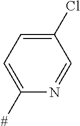 |
| P34-438 | CF₃ | 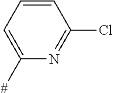 |
| P34-439 | CF₃ | 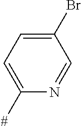 |
| P34-440 | CF₃ | 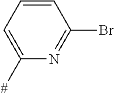 |
| P34-441 | CF₃ | 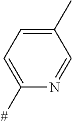 |
| P34-442 | CF₃ | 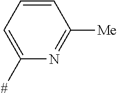 |
| P34-443 | CF₃ | 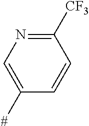 |
| P34-444 | CF₃ | 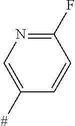 |
| P34-445 | CF₃ | 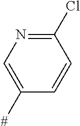 |
| P34-446 | CF₃ | 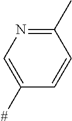 |
| P34-447 | CF₃ | 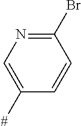 |
| P34-448 | CF₃ | 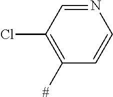 |
| P34-449 | CF₃ | 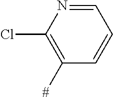 |
| P34-450 | CF₃ | 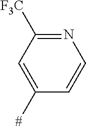 |
| P34-451 | CF₃ | 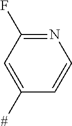 |
| P34-452 | CF₃ | 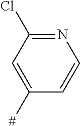 |
| P34-453 | CF₃ | 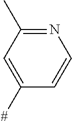 |
| P34-454 | CF₃ | 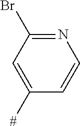 |
| P34-455 | CF₃ | 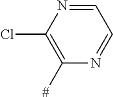 |
| P34-456 | CF₃ | 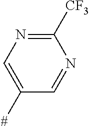 |
| P34-457 | CF₃ | 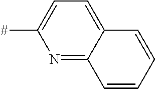 |
| P34-458 | CF₃ | 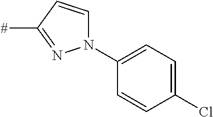 |
| P34-459 | CF₃ | 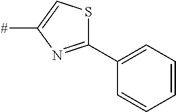 |
| P34-460 | CF₃ | 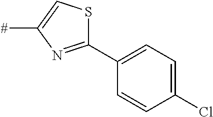 |
| P34-461 | CF₃ | 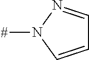 |
| P34-462 | CF₃ | 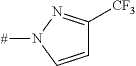 |
| P34-463 | CF₃ | 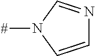 |
| P34-464 | CF₃ | 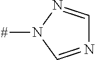 |
| P34-465 | CF₃ | 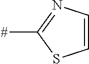 |
| P34-466 | CF₃ | 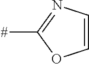 |
| P34-467 | CF₃ | 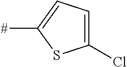 |
| P34-468 | CF₃ | 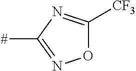 |

| | |
|---|---|
| P34-469 | 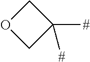 |
| P34-470 | 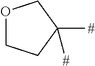 |
| P34-471 | 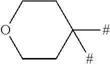 |
| P34-472 | 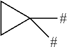 |
| P34-473 | 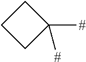 |
| P34-474 | 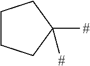 |
| P34-475 | 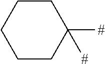 |
| P34-476 | 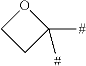 |
| P34-477 | 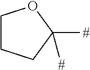 |
| P34-478 | 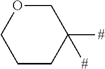 |
| P34-479 | 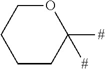 |
| P34-480 | 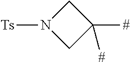 |
| P34-481 | 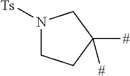 |
| P34-482 | 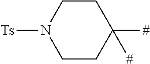 |
| P34-483 | 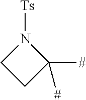 |
| P34-484 | 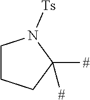 |
| P34-485 | 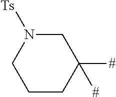 |
| P34-486 | 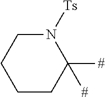 |
| P34-487 | 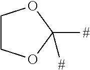 |

R^{x} in the substituent NH—SO₂—R^{x} is in each case independently selected from C₁-C₄-alkyl, C₁-C₄-haloalkyl, unsubstituted aryl and aryl that is substituted by one, two, three, four or five substituents R^{x1} independently selected from C₁-C₄-alkyl. In particular, R^{x} is in each case independently selected from C₁-C₄-alkyl and phenyl that is substituted by one, two or three R^{x1} independently selected from C₁-C₂-alkyl, more specifically R^{x} is in each case independently selected from C₁-C₄-alkyl and phenyl that is substituted by one CH₃, more specifically SO₂—R^{x} is the tosyl group (“Ts”).

R^{3a} are the possible substituents for the the acyclic moieties of R³ and the R^{3a} are in each case independently selected from halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, C₁-C₆-alkoxy, C₃-C₆-cycloalkyl, C₃-C₆-halocycloalkyl, C₁-C₄-haloalkoxy, C₁-C₆-alkylthio, CH(═O), C(═O)C₁-C₆-alkyl, C(═O)OC₁-C₆-alkyl, C(═O)NHC₁-C₆-alkyl, C(═O)N(C₁-C₆-alkyl)₂, CR^{x}═NR^{x}, a saturated or partially unsaturated three-, four-, five-, six-, seven-, eight-, nine-, or ten-membered carbocycle or heterocycle, wherein in each case one or two CH₂ groups of the carbo- and heterocycle may be replaced by a group independently selected from C(═O) and C(═S), aryl and phenoxy, wherein the aryl and phenyl groups are independently unsubstituted or substituted with substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-haloalkyl, C₁-C₄-alkoxy and C₁-C₄-haloalkoxy.

In one preferred embodiment, R^{3a} is in each case independently selected from halogen, OH, CN, C₁-C₆-alkoxy, C₁-C₆-haloalkoxy, phenyl and halogenphenyl, wherein the halogenphenyl is substituted by halogen selected from the group consisting of F, Cl and Br. In one further preferred embodiment, R^{3a} is in each case independently selected from halogen, phenyl and halogenphenyl, wherein the halogenphenyl is substituted by halogen selected from the group consisting of F, Cl and Br, in particular selected from F and Cl.

In one further preferred embodiment, R^{3a} is in each case independently selected from halogen, CN, C₃-C₆-cycloalkyl, C₃-C₆-halocycloalky, C₁-C₄-haloalkoxy, C₁-C₆-alkylthio, phenyl, wherein the phenyl is substituted by halogen selected from the group consisting of F, Cl and Br or by C₁-C₄-alkyl, C₁-C₄-haloalkyl, C₁-C₄-alkoxy and C₁-C₄-haloalkoxy. In one further preferred embodiment, R^{3a} is in each case independently selected from halogen and phenyl wherein the phenyl is substituted by halogen selected from the group consisting of F, Cl and Br, in particular selected from F and Cl.

R^{3b} are the possible substituents for the carbocycle, heterocycle, heteroaryl and aryl moieties and are independently selected from halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-haloalkyl, C₃-C₆-cycloalkyl, C₃-C₆-halocycloalky, C₁-C₄-haloalkoxy, C₁-C₆-alkylthio, C₁-C₆-haloalkylthio, C₁-C₄-alkoxy-C₁-C₄-alkyl, phenyl and phenoxy, wherein the phenyl groups are unsubstituted or substituted with substituents selected from the group consisting of halogen, OH, C₁-C₄-alkyl, C₁-C₄-haloalkyl, C₁-C₄-alkoxy and C₁-C₄-haloalkoxy.

In one preferred embodiment, R^{3b} is in each case independently selected from halogen, OH, CN, SH, C₁-C₆-alkyl, C₁-C₆-haloalkyl, C₁-C₆-alkoxy, C₁-C₆-haloalkoxy and C₁-C₆-alkylthio. In one further preferred embodiment, R^{3b} is in each case independently selected from halogen, C₁-C₆-alkoxy, C₁-C₆-haloalkoxy and C₁-C₆-haloalkyl. In one further particular embodiment, R^{3b} is in each case independently selected from C₁-C₆-alkyl, such as methyl and ethyl. In one further particular embodiment, R^{3b} is in each case independently selected from halogen, such as F, Cl and Br.

R^{4a} are the possible substituents for the the acyclic moieties of R⁴ and the R^{4a} are in each case independently selected from halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, C₁-C₆-alkoxy, C₃-C₆-cycloalkyl, C₃-C₆-halo-cycloalkyl, C₁-C₄-haloalkoxy, C₁-C₆-alkylthio, CH(═O), C(═O)C₁-C₆-alkyl, C(═O)OC₁-C₆-alkyl, C(═O)NHC₁-C₆-alkyl, C(═O)N(C₁-C₆-alkyl)₂, CR^{x}═NR^{x}, a saturated or partially unsaturated three-, four-, five-, six-, seven-, eight-, nine-, or ten-membered carbocycle or heterocycle, wherein in each case one or two CH₂ groups of the carbo- and heterocycle may be replaced by a group independently selected from C(═O) and C(═S), aryl and phenoxy, wherein the aryl and phenyl groups are independently unsubstituted or substituted with substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-haloalkyl, C₁-C₄-alkoxy and C₁-C₄-haloalkoxy.

In one preferred embodiment, R^{4a} is in each case independently selected from halogen, OH, CN, C₁-C₆-alkoxy, C₁-C₆-haloalkoxy, phenyl and halogenphenyl, wherein the halogenphenyl is substituted by halogen selected from the group consisting of F, Cl and Br. In one further preferred embodiment, R^{4a} is in each case independently selected from halogen, phenyl and halogenphenyl, wherein the halogenphenyl is substituted by halogen selected from the group consisting of F, Cl and Br, in particular selected from F and Cl.

In one preferred embodiment, R^{4a} is halogen selected from the group consisting of F, Cl and Br, in particular selected from F and Cl.

In one preferred embodiment, R^{4a} is OH.

In one preferred embodiment, R^{4a} is CN.

In still another embodiment of formula I, R^{4a} is C₁-C₆-haloalkyl, in particular C₁-C₄-haloalkyl, more specifically C₁-C₂-haloalkyl such as FCH₂, F₂CH, and CF₃,

In still another embodiment of formula I, R^{4a} is CH(═O).

In still another embodiment of formula I, R^{4a} is C(═O)C₁-C₆-alkyl, in particular C(═O)C₁-C₄-alkyl more specifically C(═O)C₁-C₂-alkyl such as COCH₃,

In still another embodiment of formula I, R^{4a} is C(═O)OC₁-C₆-alkyl, in particular C(═O)OC₁-C₄-alkyl more specifically C(═O)OC₁-C₂-alkyl such as CO₂CH₃.

In still another embodiment of formula I, R^{4a} is C(═O)NHC₁-C₆-alkyl, in particular C(═O)NHC₁-C₄-alkyl more specifically C(═O)NHC₁-C₂-alkyl such as CONHCH₃.

In still another embodiment of formula I, R^{4a} is CR^{x}═NR^{x}, such as C(CH₃)═N—OCH₃, C(CH₃)═N—OCF₃ and C(CH₃)═N—OCH₂CH₃,

In one preferred embodiment, R^{4a} is C₃-C₆-cycloalkyl, in particularly C₃-C₄-cycloalkyl, in particular CPr.

In one preferred embodiment, R^{4a} is C₃-C₆-halocycloalkyl, in particularly C₃-C₄-halocycloalkyl, more specifically C₃-halocycloalkyl such as C₃H₂Cl₂ and C₃H₂F₂.

In one preferred embodiment, R^{4a} is C₁-C₄-alkoxy, in particularly C₁-C₃-alkoxy, more specifically C₁-C₂-alkoxy, such as OCH₃.

In one preferred embodiment, R^{4a} is C₁-C₄-haloalkoxy, in particularly C₁-C₃-haloalkoxy, more specifically C₁-C₂-haloalkoxy, such as OCH₂F OCHF₂ and OCF₃.

In one preferred embodiment, R^{4a} is C₁-C₆-alkylthio, in particularly C₁-C₃-alkylthio, more specifically C₁-C₂-alkylthio, such as SCH₃. In still another embodiment of formula I, R⁴ is a partially unsaturated three-, four-, five-, six-, seven-, eight-, nine-, or ten-membered carbocycle or heterocycle, in particular three-, four-, five- or six-membered, wherein the heterocycle contains one, two, three or four heteroatoms selected from N, O and S, and wherein the carbocycle and heterocycle are unsubstituted or substituted with substituents R^{4b} as defined below. According to one embodiment thereof, the carbocycle or heterocycle is unsubstituted.

According to still a further embodiment, R⁴ is a saturated three-, four-, five-, six-, seven-, eight-, nine-, or ten-membered carbocycle or heterocycle, in particular three-, four-, five- or six-membered, wherein the heterocycle contains one, two, three or four heteroatoms selected from N, O and S, and wherein the carbocycle and heterocycle are unsubstituted or substituted with substituents R^{4b} as defined below. According to one embodiment thereof, the carbocycle or heterocycle is unsubstituted.

In still another embodiment of formula I, R⁴ is a partially unsaturated three-, four-, five-, six-, seven-, eight-, nine-, or ten-membered heterocycle, in particular three-, four-, five- or six-membered, wherein the heterocycle contains one, two, three or four heteroatoms selected from N, O and S, and wherein the heterocycle is unsubstituted or substituted by substituents R^{4b} as defined below. According to one embodiment thereof, the heterocycle is unsubstituted.

In still another embodiment of formula I, R⁴ is a saturated three-, four-, five-, six-, seven-, eight-, nine-, or ten-membered heterocycle, in particular three-, four-, five- or six-membered, wherein the heterocycle contains one, two, three or four heteroatoms selected from N, O and S, and wherein the heterocycle is unsubstituted or substituted by substituents R^{4b} as defined below.

According to one embodiment thereof, the heterocycle is unsubstituted. In still another embodiment of formula I, in the embodiments of R⁴ described above, the heterocycle contains preferably one, two or three, more specifically one or two heteroatoms selected from N, O and S. More specifically, the hetereocycle contains one heteroatom selected from N, O and S. In particular, the heterocycle contains one or two, in particular one O.

In one embodiment, R⁴ is a 4-membered saturated heterocycle which contains 1 or 2 heteroatoms, in particular 1 heteroatom, from the group consisting of N, O and S, as ring members. In one embodiment, the heterocycle contains one O as heteroatom. For example, the formed heterocycle is oxetane. According to one embodiment thereof, the heterocycle is unsubstituted, i.e. it does not carry any substituent R^{4b}. In still another embodiment of formula I, it is substituted by R^{4b}.

In still another embodiment of formula I, R^{4a} is a 5-membered saturated heterocycle which contains 1, 2 or 3, in particular 1 or 2, heteroatoms from the group consisting of N, O and S, as ring members. In one embodiment, the heterocycle contains one O as heteroatom. According to one embodiment thereof, the heterocycle is unsubstituted, i.e. it does not carry any substituent R^{4b}.

In still another embodiment of formula I, it is substituted by R^{4b}.

In still another embodiment of formula I, R⁴ is a 6-membered saturated heterocycle which contains 1, 2 or 3, in particular 1 or 2, heteroatoms from the group consisting of N, O and S as ring members. According to one embodiment thereof, the heterocycle is unsubstituted, i.e. it does not carry any substituent R^{4b}. In still another embodiment of formula I, it is substituted by R^{4b}.

According to one specific embodiment thereof, said 6-membered saturated heterocycle contains 1 or 2, in particular 1, heteroatom(s) O. According to one embodiment thereof, the respective 6-membered heterocycle is unsubstituted, i.e. it does not carry any substituent R^{4b}. In still another embodiment of formula I, it is substituted by R^{4b}.

In still another embodiment of formula I, R^{4a} is a partially unsaturated three-, four-, five-, six-, seven-, eight-, nine-, or ten-membered carbocycle, in particular three-, four-, five- or six-membered, wherein the carbocycle is unsubstituted or substituted by substituents R^{4b} as defined below. According to one embodiment thereof, the carbocycle is unsubstituted.

In still another embodiment of formula I, R^{4a} is a saturated three-, four-, five-, six-, seven-, eight-, nine-, or ten-membered carbocycle, in particular three-, four-, five- or six-membered, wherein the carbocycle is unsubstituted or substituted by substituents R^{4b} as defined below. According to one embodiment thereof, the carbocycle is unsubstituted.

In one embodiment, R^{4a} is a 3-membered saturated carbocycle. According to one embodiment thereof, the carbocycle is unsubstituted, i.e. it does not carry any substituent R^{4b}. In still another embodiment of formula I, it is substituted by R^{4b}.

In one embodiment, R^{4a} is a 4-membered saturated carbocycle. According to one embodiment thereof, the carbocycle is unsubstituted, i.e. it does not carry any substituent R^{4b}. In still another embodiment of formula I, it is substituted by R^{4b}.

In one embodiment, R^{4a} is a 5-membered saturated carbocycle. According to one embodiment thereof, the carbocycle is unsubstituted, i.e. it does not carry any substituent R^{4b}. In still another embodiment of formula I, it is substituted by R^{4b}.

In one embodiment, R^{4a} is a 6-membered saturated carbocycle. According to one embodiment thereof, the carbocycle is unsubstituted, i.e. it does not carry any substituent R^{4b}. In still another embodiment of formula I, it is substituted by R^{4b}.

In one further preferred embodiment, R^{4a} is in each case independently selected from halogen, CN, C₃-C₆-cycloalkyl, C₃-C₆-halocycloalky, C₁-C₄-haloalkoxy, C₁-C₆-alkylthio, phenyl, carbo- and heterocycle; wherein the phenyl is substituted by halogen selected from the group consisting of F, Cl and Br or by C₁-C₄-alkyl, C₁-C₄-haloalkyl, C₁-C₄-alkoxy and C₁-C₄-haloalkoxy. In one further preferred embodiment, R^{4a} is in each case independently selected from halogen and phenyl wherein the phenyl is substituted by halogen selected from the group consisting of F, Cl and Br, in particular selected from F and Cl.

R^{4b} are the possible substituents for the carbocycle, heterocycle, heteroaryl and aryl moieties and are independently selected from halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-haloalkyl, C₃-C₆-cycloalkyl, C₃-C₆-halocycloalky, C₁-C₄-haloalkoxy, C₁-C₆-alkylthio, C₁-C₆-haloalkylthio, C₁-C₄-alkoxy-C₁-C₄-alkyl, phenyl and phenoxy, wherein the phenyl groups are unsubstituted or substituted with substituents selected from the group consisting of halogen, OH, C₁-C₄-alkyl, C₁-C₄-alkoxy-C₁-C₄-alkyl, and C₁-C₄-haloalkoxy.

In one preferred embodiment, R^{4b} is in each case independently selected from halogen, OH, CN, SH, C₁-C₆-alkyl, C₁-C₆-haloalkyl, C₁-C₆-alkoxy, C₁-C₆-haloalkoxy and C₁-C₆-alkylthio. In one further preferred embodiment, R^{4b} is in each case independently selected from halogen, C₁-C₆-alkkoxy and C₁-C₆-haloalkyl, C₁-C₆-haloalkoxy, such as OCH₃, OCH₂F, OCHF₂ and OCF₃ In one further particular embodiment, R^{4b} is in each case independently selected from C₁-C₆-alkyl, such as methyl and ethyl. In one further particular embodiment, R^{4b} is in each case independently selected from halogen, such as F, Cl and Br.

R⁵ is H.

R⁶ is H.

The group W together with the partially saturated pyridine ring it is attached forms ring A as shown in structure I-A below

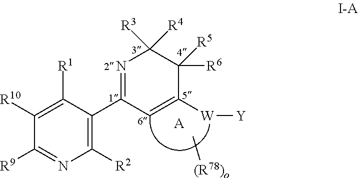

Wherein the ring A-W—Y is selected from below groups:

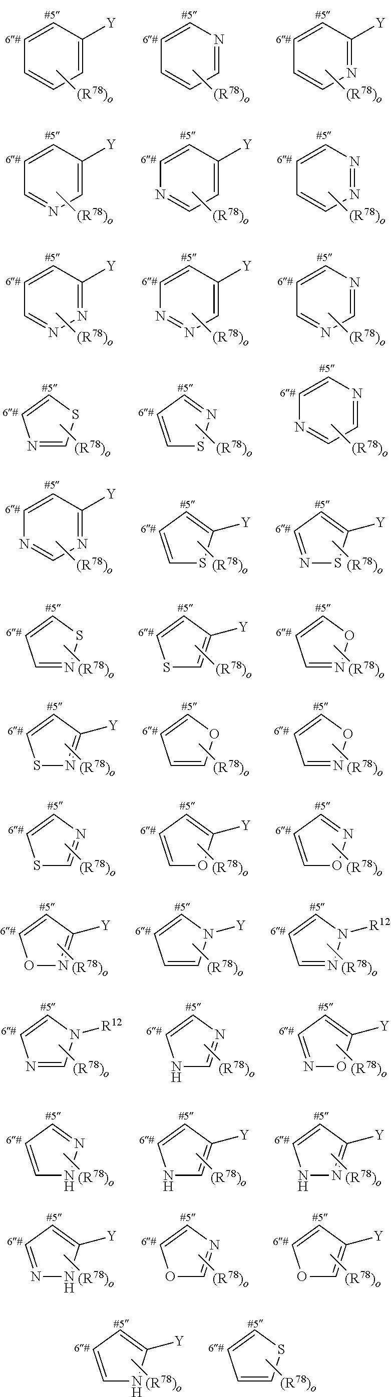

wherein, the positions of the rings marked with “#” represents the connection points (carbon atoms 5″ and 6″ in formula I) with the remaining skeleton of the compounds of formula and wherein the ring A-W—Y is substituted with (R⁷⁸)ₒ, wherein o is 0, 1, 2 or 3; and R⁷⁸ are independently selected from halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, CH(═O), C(═O)C₁-C₆-alkyl, C(═O)NH(C₁-C₆-alkyl), CR′═NOR″, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₂-C₆-alkenyloxy, C₂-C₆-alkynyloxy, C₃-C₆-cycloalkyl, C₃-C₆-cycloalkenyl, three-, four-, five- or six-membered saturated or partially unsaturated heterocycle, five- or six-membered heteroaryl and phenyl; wherein the heterocycle or heteroaryl contains one, two or three heteroatoms selected from N, O and S; wherein R′ and R″ are independently selected from H, C₁-C₄-alkyl, C₂-C₆-alkenyl, C₂-C₆-alkynyl, C₃-C₆-cycloalkyl, saturated or partially unsaturated three-, four-, five-, six-, seven-, eight-, nine-, or ten-membered heterocycle, five- or six-membered heteroaryl or aryl; wherein the heterocycle or heteroaryl contains one, two or three heteroatoms selected from N, O and S, and wherein R′ and/or R″ are independently unsubstituted or substituted with R′″ independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₃-C₆-cycloalkyl, C₃-C₆-halocycloalky and phenyl; and wherein R^{x} is defined above; and wherein the acyclic moieties of R⁷⁸ are unsubstituted or substituted with identical or different groups R^{78b} which independently of one another are selected from:

R^{78a} halogen, OH, CN, C₁-C₆-alkoxy, C₃-C₆-cycloalkyl, C₃-C₆-cycloalkenyl, C₃-C₆-halocycloalkyl, C₃-C₆-halocycloalkenyl, C₁-C₄-haloalkoxy, C₁-C₆-alkylthio, five- or six-membered heteroaryl, phenyl and phenoxy, wherein the heterorayl, phenyl and phenoxy group is unsubstituted or substituted with R^{78aa} selected from the group consisting of halogen, OH, C₁-C₄-alkyl, C₁-C₄-haloalkyl, C₁-C₄-alkoxy and C₁-C₄-haloalkoxy;

wherein the alicyclic, phenyl, heterocyclic and heteroaryl moieties of R⁷⁸ are unsubstituted or substituted with identical or different groups R^{78b} which independently of one another are selected from:

R^{78b} halogen, OH, CN, C₁-C₄-alkyl, C₁-C₄-alkoxy, C₁-C₄-haloalkyl, C₃-C₆-cycloalkyl, C₃-C₆-halo-cycloalkyl, C₁-C₄-haloalkoxy and C₁-C₆-alkylthio.

Y is selected from F, Cl, Br, I, cyano, nitro, C₁-C₆-alkyl, C₁-C₆-haloalkyl, C₂-C₆-alkenyl, C₁-C₆-haloalkoxy, C₂-C₆-alkynyl, C₃-C₆-cycloalkyl, C₃-C₆-cycloalkenyl, —O(Y¹), —S(O), (Y²), —N(Y³)(Y⁴), C₁-C₆-haloalkyl, C₂-C₆-haloalkenyl, C₂-C₆-haloalkynyl, —CO(Y⁵), —C(Y⁶)═NO(Y⁷); wherein the acyclic moieties of Y are unsubstituted or substituted by Rma and the alicyclic, phenyl and heteroaryl moieties of Y are unsubstituted or substituted by R^{78b}.

In one embodiment of formula I, Y is F, Cl, Br, I preferably F.

In one embodiment of formula I, Y is F, Cl, Br, I preferably Cl.

In one embodiment of formula I, Y is F, Cl, Br, I preferably Br.

In one embodiment of formula I, Y is CN.

In one embodiment of formula I, Y is OH.

In one embodiment of formula I, Y is NO₂.

In still another embodiment of formula I, Y is C₁-C₆-alkyl, such as CH₃, C₂H₅, n-propyl, i-propyl, n-butyl, i-butyl, tert-butyl, n-pentyl or i-pentyl, preferably methyl.

In still another embodiment of formula I, Y is C₁-C₆-alkyl, such as CH₃.

In still another embodiment of formula I, Y is C₁-C₆-haloalkyl, in particular C₁-C₄-haloalkyl, more specifically C₁-C₂-haloalkyl, such as CF₃, CCl₃, FCH₂, ClCH₂, F₂CH, Cl₂CH, CF₃CH₂, CCl₃CH₂ or CF₂CHF₂ preferably FCH₂, F₂CH and CF₃.

In still another embodiment of formula I, Y is C₂-C₆-alkenyl, such as CH═CH₂.

In still another embodiment of formula I, Y is C₂-C₆-haloalkenyl, in particular C₂-C₄-haloalkenyl, more specifically C₂-C₃-haloalkenyl.

According to still a further embodiment of formula I, Y is C₂-C₆-alkynyl, in particular C₂-C₄-alkynyl, more specifically C₂-C₃-alkynyl, such as C≡CH.

According to still a further embodiment of formula I, Y is C₂-C₆-haloalkynyl, in particular C₂-C₄-haloalkynyl, more specifically C₂-C₃-haloalkynyl.

In still another embodiment of formula I, Y is cycloalkyl such as cyclopropyl, cyclobutyl, cyclopentyl, cyclohexyl, cycloheptyl, preferably cyclopropyl.

In still another embodiment of formula I, Y is O(Y¹), wherein Y¹ is C₁-C₆-alkyl, in particular C₁-C₄-alkyl, more specifically C₁-C₂-alkyl, such as OCH₃ or OCH₂CH₃.

In still another embodiment of formula I, Y is CN, C₁-C₆-alkyl, C₁-C₆-haloalkyl, C₂-C₆-haloalkenyl C₁-C₆-alkoxy or C₁-C₆-haloalkoxy.

In still another embodiment of formula I, Y is C₁-C₆-alkyl, C₂-C₆-alkenyl, C₂-C₆-alkynyl.

In still another embodiment of formula I, Y is O(Y¹), wherein Y¹ is C₁-C₆-haloalkyl, in particular C₁-C₄-haloalkyl, more specifically C₁-C₂-haloalkyl, such as OCH₂F, OCHF₂, OCF₃ or OCH₂CF₃.

In still another embodiment of formula I, Y is O(Y¹), wherein Y¹ is C₁-C₆-alkenyl, in particular C₁-C₄-alkenyl, more specifically C₁-C₃-alkenyl, such as OCH═CH₂ or OCH₂CH═CH₂.

In still another embodiment of formula I, Y is O(Y¹), wherein Y¹ is C₁-C₆-alkynyl, in particular C₁-C₄-alkynyl, more specifically C₁-C₃-alkynyl, such as OC≡CH or OCH₂C≡CH.

In still another embodiment of formula I, Y is O(Y¹), wherein Y¹ is C₃-C₆-cycloalkyl, in particular C₃-C₄-cycloalky, more specifically cyclopropyl such as OCPr or OCH₂CPr.

In still another embodiment of formula I, Y is O(Y¹), wherein Y¹ is C₁-C₆-haloalkenyl, in particular C₁-C₄-haloalkenyl, more specifically C₁-C₃-haloalkenyl, such as OCH═CHF, OCH═CHCl, OCH═CF₂, OCH═CCl₂, OCH₂CH═CHF, OCH₂CH═CHCl, OCH₂CH═CF₂, OCH₂CH═CCl₂. OCH₂CF═CF₂, OCH₂CCl═CCl₂. OCF₂CF═CF₂ orO CCl₂CCl═CCl₂.

In still another embodiment of formula I, Y is O(Y¹), wherein Y¹ is C₁-C₆-haloalkynyl, in particular C₁-C₄-haloalkynyl, more specifically C₁-C₃-haloalkynyl, such as OC≡CCl or OCH₂ C≡CCl.

In still another embodiment of formula I, Y is O(Y¹), wherein Y¹ is is aryl, in particular phenyl, wherein the aryl or phenyl moiety in each case is unsubstituted or substituted by identical or different groups R^{78b} which independently of one another are selected from halogen, C₁-C₂-alkyl, C₁-C₂-alkoxy, C₁-C₂-halogenalkyl and C₁-C₂-halogenalkoxy, in particular CN, F, Cl, Br, CH₃, CHF₂, OCH₃, OCHF₂, CF₃ and OCF₃. According to one embodiment, R⁷⁸ is unsubstituted phenyl. According to another embodiment, R⁷⁸ is phenyl, that is substituted by one, two or three, in particular one, halogen, in particular selected from F, Cl and Br, more specifically selected from F and Cl.

In still another embodiment of formula I, Y is O(Y¹), wherein Y¹ is a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-thiol-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl.

In still another embodiment of formula I Y is S(O)z(Y²) such as S(O)z-C₁-C₆-alkyl such as SCH₃, S(═O) CH₃, S(O)₂CH₃.

In still another embodiment of formula I, Y is N(Y³)(Y⁴) such as NH₂.

In still another embodiment of formula I, Y is N(Y³)(Y⁴) such as NH(C₁-C₄-alkyl), in particular NH(CH₃), NH(C₂H₅).

In still another embodiment of formula I, Y is N(Y³)(Y⁴) such as, N(C₁-C₄-alkyl)₂, in particular NH(CH₃)₂, NH(C₂H₅)₂.

In still another embodiment of formula I, Y is N(Y³)(Y⁴) such as, NH(C(═O)(C₁-C₄-alkyl), in particular NH(C(═O)(CH₃), NH(C(═O)(C₂H₅).

In still another embodiment of formula I, Y is N(Y³)(Y⁴) such as N(C(═O)(C₁-C₄-alkyl)₂, in particular N(C(═O)(CH₃)₂, N(C(═O)(C₂H₅)₂.

In a further specific embodiment of formula I, Y is N(Y³)(Y⁴) such as NH—SO₂—R^{x} such as NH—SO₂—CH₃, NH—SO₂—CH₂—CH₃, NH—SO₂—CF₃ or NH—SO₂-Ts.

In a further specific embodiment of formula I, Y is CO(Y⁵) such as CH(═O), C(═O)C₁-C₆-alkyl, C(═O)O(C₁-C₆-alkyl) or C(═O)NH(C₁-C₆-alkyl), wherein alkyl is CH₃, C₂H₅, n-propyl, i-propyl, n-butyl, i-butyl, tert-butyl, n-pentyl or i-pentyl.

In a further specific embodiment of formula I, Y is C(Y⁶)═NO(Y⁷) such as C(CH₃)═NOCH₃, C(CH₃)═NOCH₂CH₃ or C(CH₃)═NOCF₃.

Wherein if ring A represents phenyl ring Y does not represent F.

Wherein if ring A represents phenyl ring Y does not represent halogen.

According to the invention, there can be zero, one, two or three R⁷⁸ present, namely for o is 0, 1, 2 or 3.

In still another embodiment of formula 1₇o is 0.

In still another embodiment of formula I, o is 1.

In still another embodiment of formula I, o is 2 or 3. According to one specific embodiment thereof, o is 2, In still another embodiment of formula I, o is 3.

For every R⁷⁸ that is present in the compounds of formula I, the following embodiments and preferences apply independently of the meaning of any other R⁷⁸ that may be present in the ring. Furthermore, the particular embodiments and preferences given herein for R⁷⁸ apply independently for each of o=1, o=2 and o=3.

According to one embodiment of formula I, R⁷⁸ is selected from the group consisting of halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, C₁-C₆-alkoxy, C₁-C₆-halogenalkoxy.

In one embodiment of formula I, R⁷⁸ is halogen, in particular F, Cl, Br or I, more specifically F, Cl or Br, in particular F or Cl.

In still another embodiment of formula I, R⁷⁸ is F.

In still another embodiment of formula I, R⁷⁸ is Cl.

In still another embodiment of formula I, R⁷⁸ is Br.

In still another embodiment of formula I, R⁷⁸ is OH.

In still another embodiment of formula I, R⁷⁸ is CN.

According to still another embodiment of formula I, R⁷⁸ is NO₂.

According to still another embodiment of formula I, R⁷⁸ is SH.

According to still another embodiment of formula I, R⁷⁸ is NH₂.

According to still another embodiment of formula I, R⁷⁸ is NH(C₁-C₄-alkyl), in particular NH(CH₃), NH(C₂H₅).

According to still another embodiment of formula I, R⁷⁸ is N(C₁-C₄-alkyl)₂, in particular NH(CH₃)2, NH(C₂H₅)₂.

According to still another embodiment of formula I, R⁷⁸ is NH(C(═O)(C₁-C₄-alkyl), in particular NH(C(═O)(CH₃), NH(C(═O)(C₂H₅).

According to still another embodiment of formula I, R⁷⁸ is N(C(═O)(C₁-C₄-alkyl)₂, in particular N(C(═O)(CH₃)₂, N(C(═O)(C₂H₅)₂.

According to a further specific embodiment of formula I, R⁷⁸ is NH—SO₂—R^{x} such as NH—SO₂—CH₃, NH—SO₂—CH₂—CH₃, NH—SO₂—CF₃ or NH—SO₂-Ts.

According to a further specific embodiment of formula I, R⁷⁸ is CH(═O), C(═O)C₁-C₆-alkyl, C(═O)O(C₁-C₆-alkyl) or C(═O)NH(C₁-C₆-alkyl), wherein alkyl is CH₃, C₂H₅, n-propyl, i-propyl, n-butyl, i-butyl, tert-butyl, n-pentyl or i-pentyl.

According to a further specific embodiment of formula I, R⁷⁸ is CR′═NOR″ such as C(CH₃)═NOCH₃, C(CH₃)═NOCH₂CH₃ or C(CH₃)═NOCF₃.

In still another embodiment of formula I, R⁷⁸ is C₁-C₆-alkyl, in particular C₁-C₄-alkyl, such as CH₃, or C₂H₅, in particular CH₃.

In still another embodiment of formula I, R⁷⁸ is C₁-C₆-haloalkyl, in particular C₁-C₄-haloalkyl, such as CF₃, CHF₂, CH₂F, CCl₃, CHCl₂ and CH₂Cl.

According to still a further embodiment, R⁷⁸ is C₂-C₆-alkenyl, in particular C₂-C₄-alkenyl, such as CH═CH₂ or CH₂ CH═CH₂.

According to still a further embodiment, R⁷⁸ is C₃-C₆-cycloalkyl-C₂-C₆-alkenyl, in particular C₃-C₆-cycloalkyl-C₂-C₄-alkenyl, more specifically C₃-C₆-cycloalkyl-C₂-C₃-alkenyl, such as C₃H₅-CH═CH₂.

In still another embodiment of formula I, R⁷⁸ is C₂-C₆-haloalkenyl, in particular C₂-C₄-haloalkenyl, more specifically C₂-C₃-haloalkenyl, such as CH═CHF, CH═CHCl, CH═CF₂, CH═CCl₂, CH₂CH═CHF, CH₂CH═CHCl, CH₂CH═CF₂, CH₂CH═CCl₂. CH₂CF═CF₂, CH₂CCl═CCl₂. CF₂CF═CF₂ or CCl₂CCl═CCl₂.

According to still a further embodiment of formula I, R⁷⁸ is C₂-C₆-alkynyl, in particular C₂-C₄-alkynyl, more specifically C₂-C₃-alkynyl, such as C≡CH or CH₂C≡CH.

According to still a further embodiment of formula I, R⁷⁸ is C₂-C₆-haloalkynyl, in particular C2-C₄-haloalkynyl, more specifically C₂-C₃-haloalkynyl, such as C≡CCl or CH₂C≡CCl.

In still another embodiment of formula I, R⁷⁸ is C₁-C₆-alkoxy, in particular C₁-C₄-alkoxy, more specifically C₁-C₂-alkoxy such as OCH₃ or OCH₂CH₃.

In still another embodiment of formula I, R⁷⁸ is C₁-C₆-haloalkoxy, in particular C₁-C₄-haloalkoxy, more specifically C₁-C₂-haloalkoxy such as OCF₃, OCHF₂, OCH₂F, OCCl₃, OCHCl₂, OCH₂Cl and OCF₂CHF₂, in particular OCF₃, OCHF₂ and OCF₂CHF₂.

According to a further specific embodiment of formula I, R⁷⁸ is C₂-C₆-alkenyloxy, in particular C₂-C₄-alkenyloxy, more specifically C₁-C₂-alkenyloxy such as OCH═CH₂, OCH₂CH═CH₂.

According to a further specific embodiment of formula I, R⁷⁸ is C₂-C₆-alkynyloxy, in particular C₂-C₄-alkynyloxy, more specifically C₁-C₂-alkynyloxy such as OC≡CH

According to still another embodiment of formula I R⁷⁸ is C₃-C₆-cycloalkyl, in particular cyclopropyl.

According to still another embodiment of formula I, R⁷⁸ is C₃-C₆-halocycloalkyl. In a special embodiment R¹ is fully or partially halogenated cyclopropyl.

According to still another embodiment of formula I R⁷⁸ is C₃-C₆-cycloalkenyl, in particular cyclopropenyl.

According to one embodiment, R⁷⁸ is a 3-membered saturated carbocycle. According to one embodiment thereof, the carbocycle is unsubstituted, i.e. it does not carry any substituent R^{78b}.

According to still another embodiment of formula I, it is substituted by R^{78b}.

According to one embodiment, R⁷⁸ is a 4-membered saturated carbocycle. According to one embodiment thereof, the carbocycle is unsubstituted, i.e. it does not carry any substituent R^{78b}.

According to still another embodiment of formula I, it is substituted by R^{78b}.

According to one embodiment, R⁷⁸ is a 5-membered saturated carbocycle. According to one embodiment thereof, the carbocycle is unsubstituted, i.e. it does not carry any substituent R^{78b}.

According to still another embodiment of formula I, it is substituted by R^{78b}.

According to one embodiment, R⁷⁸ is a 6-membered saturated carbocycle. According to one embodiment thereof, the carbocycle is unsubstituted, i.e. it does not carry any substituent R^{78b}.

According to still another embodiment of formula I, it is substituted by R^{78b}.

According to still another embodiment of formula I, R⁷⁸ is a partially unsaturated three-, four-, five-, six-, seven-, eight-, nine-, or ten-membered heterocycle, in particular three-, four-, five- or six-membered, wherein the heterocycle contains one, two, three or four heteroatoms selected from N, O and S, and wherein the heterocycle is unsubstituted or substituted by substituents R^{78b} as defined below. According to one embodiment thereof, the heterocycle is unsubstituted.

According to still another embodiment of formula I, R⁷⁸ is a saturated three-, four-, five-, six-, seven-, eight-, nine-, or ten-membered heterocycle, in particular three-, four-, five- or six-membered, wherein the heterocycle contains one, two, three or four heteroatoms selected from N, O and S, and wherein the heterocycle is unsubstituted or substituted by substituents R^{78b} as defined below. According to one embodiment thereof, the heterocycle is unsubstituted.

According to still another embodiment of formula I, in the embodiments of R⁷⁸ described above, the heterocycle contains preferably one, two or three, more specifically one or two heteroatoms selected from N, O and S. More specifically, the hetereocycle contains one heteroatom selected from N, O and S. In particular, the heterocycle contains one or two, in particular one O.

According to one embodiment, R⁷⁸ is a 4-membered saturated heterocycle which contains 1 or 2 heteroatoms, in particular 1 heteroatom, from the group consisting of N, O and S, as ring members. According to one embodiment, the heterocycle contains one O as heteroatom. For example, the formed heterocycle is oxetane. According to one embodiment thereof, the heterocycle is unsubstituted, i.e. it does not carry any substituent R^{78b}. According to still another embodiment of formula I, it is substituted by R⁷⁸b.

According to still another embodiment of formula I, R⁷⁸ is a 5-membered saturated heterocycle which contains 1, 2 or 3, in particular 1 or 2, heteroatoms from the group consisting of N, O and S, as ring members. According to one embodiment, the heterocycle contains one O as heteroatom. According to one embodiment thereof, the heterocycle is unsubstituted, i.e. it does not carry any substituent R^{78b}. According to still another embodiment of formula I, it is substituted by R^{78b}.

According to still another embodiment of formula I, R⁷⁸ is a 6-membered saturated heterocycle which contains 1, 2 or 3, in particular 1 or 2, heteroatoms from the group consisting of N, O and S as ring members. According to one embodiment thereof, the heterocycle is unsubstituted, i.e. it does not carry any substituent R^{78b}. According to still another embodiment of formula I, it is substituted by R^{78b}. According to one specific embodiment thereof, said 6-membered saturated heterocycle contains 1 or 2, in particular 1, heteroatom(s) O. According to one embodiment thereof, the respective 6-membered heterocycle is unsubstituted, i.e. it does not carry any substituent R^{78b}. According to still another embodiment of formula I, it is substituted by R^{78b}.

According to still another embodiment of formula I, R⁷⁸ is phenyl-C₁-C₆-alkyl, such as phenyl-CH₂, wherein the phenyl moiety in each case is unsubstituted or substituted by one, two or three identical or different groups R^{78b} which independently of one another are selected from halogen, C₁-C₂-alkyl, C₁-C₂-alkoxy, C₁-C₂-halogenalkyl and C₁-C₂-halogenalkoxy, in particular CN, F, Cl, Br, CH₃, OCH₃, CHF₂, CF₃ OCHF₂, and OCF₃.

In still another embodiment of formula I, R⁷⁸ is unsubstituted phenyl or phenyl that is substituted by one, two, three or four R^{78b}, as defined and preferably herein. In particular, R⁷⁸ is unsubstituted phenyl or phenyl that is substituted by one, two, three or four R^{78b}, as defined herein. In one embodiment R⁷⁸ is unsubstituted phenyl.

According to still another embodiment of formula I, R⁷⁸ is a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl.

In still another embodiment of formula I, R⁷⁸ is in each case independently selected from halogen, CN, C₁-C₆-alkyl, C₂-C₆-alkenyl, C₂-C₆-alkynyl, C₁-C₆-alkoxy, C₃-C₆-alkenyloxy, C₃-C₆-alkynyloxy, C₃-C₆-cycloalkyl, three-, four-, five- or six-membered saturated or partially unsaturated heterocycle, five- or six-membered heteroaryl and phenyl; wherein the heterocycle or heteroaryl contains one, two or three heteroatoms selected from N, O and S; and wherein the acyclic moieties of R⁷⁸ are unsubstituted or substituted with identical or different groups R^{78a} as defined and preferably defined herein, and wherein the heterocyclic, alicyclic, phenyl and heteroaryl moieties of R⁷⁸ are unsubstituted or substituted with identical or different groups R^{78b} as defined and preferably defined herein.

In still another embodiment of formula I, R⁷⁸ is in each case independently selected from halogen, CN, C₁-C₆-alkyl, C₂-C₆-alkenyl, C₂-C₆-alkynyl, C₁-C₆-alkoxy, C₁-C₆-haloalkoxy, C₃-C₆-alkenyloxy, C₃-C₆-alkynyloxy, C₃-C₆-cycloalkyl, three-, four-, five- or six-membered saturated or partially unsaturated heterocycle, five- or six-membered heteroaryl and phenyl; wherein the heterocycle or heteroaryl contains one, two or three heteroatoms selected from N, O and S; and wherein the acyclic moieties of R⁷⁸ are unsubstituted or substituted with identical or different groups R^{78a} as defined and preferably defined herein, and wherein the heterocyclic, alicyclic, phenyl and heteroaryl moieties of R⁷⁸ are unsubstituted or substituted with identical or different groups R^{78b} as defined and preferably defined herein. Accordingto one specific embodiment, the aliphatic and cyclic moieties of R⁷⁸ are unsubstituted, according to another embodiment, the acyclic moieties of R⁷⁸ substituted with identical or different groups R^{78a} as defined and preferably defined herein.

In still another embodiment of formula I, R⁷⁸ is in each case independently selected from halogen, CN, C₁-C₆-alkyl, C₂-C₆-alkenyl, C₂-C₆-alkynyl, C₁-C₆-alkoxy, C₃-C₆-alkenyloxy, C₃-C₆-alkynyloxy and C₃-C₆-cycloalkyl, wherein the acyclic moieties of R⁷⁸ are unsubstituted or substituted with identical or different groups ^{R78a} as defined and preferably defined herein, and wherein the cycloalkyl moieties of R⁷⁸ are unsubstituted or substituted with identical or different groups R^{78b} as defined and preferably defined herein.

In still another embodiment of formula I, R⁷⁸ is in each case independently selected from halogen, CN, C₁-C₆-alkyl, C₂-C₆-alkenyl, C₂-C₆-alkynyl, C₁-C₆-alkoxy, C₁-C₆-haloalkoxy, C₃-C₆-alkenyloxy, C₃-C₆-alkynyloxy and C₃-C₆-cycloalkyl, wherein the acyclic moieties of R⁷⁸ are unsubstituted or substituted with identical or different groups R^{78a} as defined and preferably defined herein, and wherein the cycloalkyl moieties of R⁷⁸ are unsubstituted or substituted with identical or different groups R^{78b} as defined and preferably defined herein. Accordingto one specific embodiment, the aliphatic and cyclic moieties of R⁷⁸ are not further substituted, according to another embodiment, the acyclic moieties of R⁷⁸ carry one, two, three or four identical or dif-ferent groups R^{78a} as defined and preferably defined herein.

In still another embodiment of formula I, R⁷⁸ is in each case independently selected from halogen, C₁-C₆-alkyl and C₁-C₆-alkoxy, wherein the acyclic moieties of R⁷⁸ are unsubstituted or substituted with identical or different groups R^{78a} defined and preferably defined herein.

In still another embodiment of formula I, R⁷⁸ is in each case independently selected from halogen, C₁-C₆-alkyl, C₁-C₆-alkoxy and C₁-C₆-haloalkoxy, wherein the acyclic moieties of R⁷⁸ are unsubstituted or substituted with identical or different groups R^{78a} defined and preferably defined herein. Accordingto one specific embodiment, the aliphatic and cyclic moieties of R⁷⁸ are not further substituted, according to another embodiment, the acyclic moieties of R⁷⁸ carry one, two, three or four identical or different groups ^{R78a} as defined and preferably defined herein.

R^{78a} are the possible substituents for the acyclic moieties of R⁷⁸. R^{78a} is independently selected from halogen, OH, CN, C₁-C₆-alkoxy, C₃-C₆-cycloalkyl, C₃-C₆-cycloalkenyl, C₃-C₆-halocycloalkyl, C₃-C₆-halocyhalocycloalkenyl, C₁-C₄-haloalkoxy, C₁-C₆-alkylthio, five- or six-membered heteroaryl, phenyl and phenoxy, wherein the heteroraryl, phenyl and phenoxy group is unsubstituted or unsubstituted or substituted with R^{78aa} selected from the group consisting of halogen, OH, C₁-C₄-alkyl, C₁-C₄-haloalkyl, C₁-C₄-alkoxy and C₁-C₄-haloalkoxy.

According to one embodiment R^{78a} is independently selected from halogen, C₁-C₆-alkoxy, C₃-C₆-cycloalkyl, C₃-C₆-halocycloalkyl and C₁-C₄-haloalkoxy. Specifically, R^{78a} is independently selected from F, Cl, Br, I, C₁-C₂-alkoxy, cyclopropyl, 1-F-cyclopropyl, 1-Cl-cyclopropyl and C₁-C₂-haloalkoxy.

In still another embodiment of formula I, R^{78a} is independently halogen, in particular selected from F, Cl, Br and I, more specifically F, Cl and Br.

R^{78b} are the possible substituents for the cycloalkyl, heterocyclyl, heteroaryl and phenyl moieties of R⁷⁸. R^{78b} according to the invention is independently selected from halogen, OH, CN, C₁-C₄-alkyl, C₁-C₄-alkoxy, C₁-C₄-haloalkyl, C₃-C₆-cycloalkyl, C₃-C₆-halocycloalky, C₁-C₄-haloalkoxy and C₁-C₆-alkylthio.

According to one embodiment thereof R^{78b} is independently selected from halogen, CN, C₁-C₄-alkyl, C₁-C₄-alkoxy, C₁-C₄-haloalkyl and C₁-C₄-haloalkoxy, in particular halogen, C₁-C₄-alkyl and C₁-C₄-alkoxy. Specifically, R^{78b} is independently selected from F, Cl, CN, CH₃, OCH₃ and halogenmethoxy.

Particularly preferred embodiments of the ring A-W—Y, optionally substituted by (R⁷⁸)ₒ, according to the invention are in Table P78 below, wherein each line of lines P78-1 to P78-38 corresponds to one particular embodiment of the invention, wherein P78-1 to P78-38 are also in any combination with one another a preferred embodiment of the present invention. Thereby, the positions of the pheny or heteroaryls marked with “#” represents the connection points (carbon atoms 5″ and 6″ in formula I) with the remaining skeleton of the compounds of formula I:

TABLE P78

| No. | A |
|---|---|
| P78-1 | 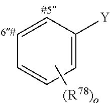 |
| P78-2 | 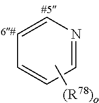 |
| P78-3 | 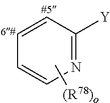 |
| P78-4 | 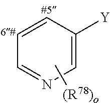 |
| P78-5 | 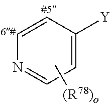 |
| P78-6 | 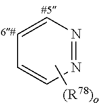 |
| P78-7 | 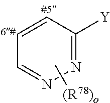 |
| P78-8 | 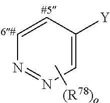 |
| P78-9 | 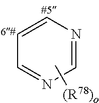 |
| P78-10 | 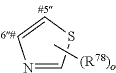 |
| P78-11 | 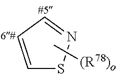 |
| P78-12 | 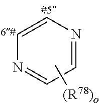 |
| P78-13 | 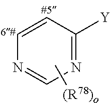 |
| P78-14 | 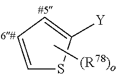 |
| P78-15 | 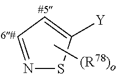 |
| P78-16 | 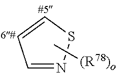 |
| P78-17 | 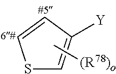 |
| P78-18 | 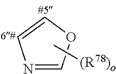 |
| P78-19 | 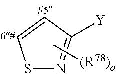 |
| P78-20 | 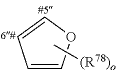 |
| P78-21 | 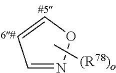 |
| P78-22 | 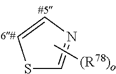 |
| P78-23 | 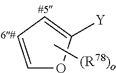 |
| P78-24 | 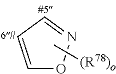 |
| P78-25 | 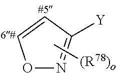 |
| P78-26 | 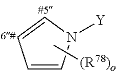 |
| P78-27 | 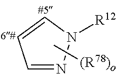 |
| P78-28 | 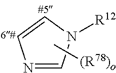 |
| P78-29 | 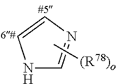 |
| P78-30 | 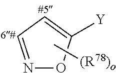 |
| P78-31 | 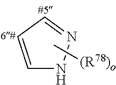 |
| P78-32 | 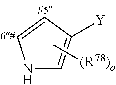 |
| P78-33 | 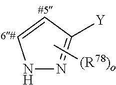 |
| P78-34 | 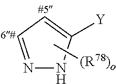 |
| P78-35 | 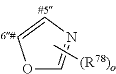 |
| P78-36 | 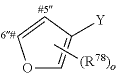 |
| P78-37 | 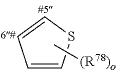 |
| P78-38 | 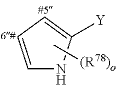 |

Preferred embodiments of the formula I are the following compounds I.A, I.B, I.C, I.D, I.E, I.F, I.G, I.H, I.I, I.J, I.K and I.Ka. In these formulae, the substituents R¹, R², R³, R⁴, R⁵, R⁶, R9, R¹⁰, R⁷⁸ and o are independently as defined for formula I:

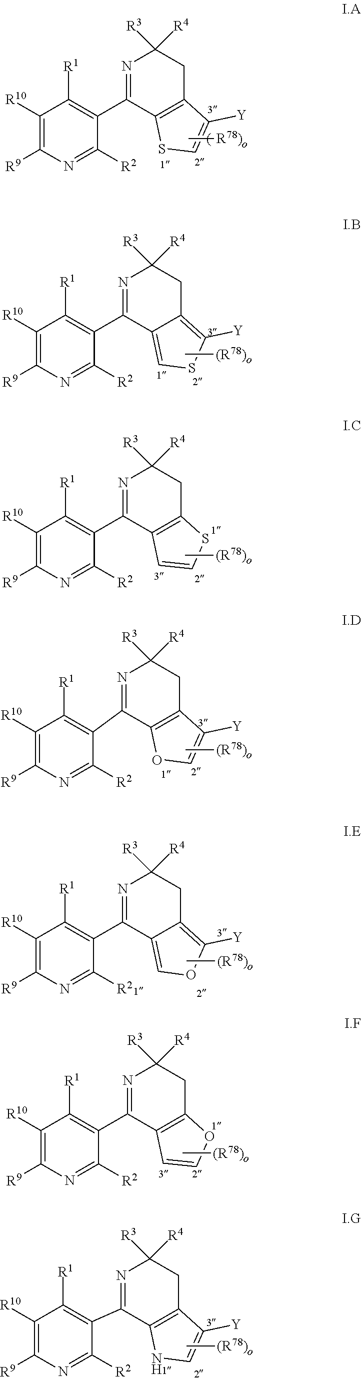

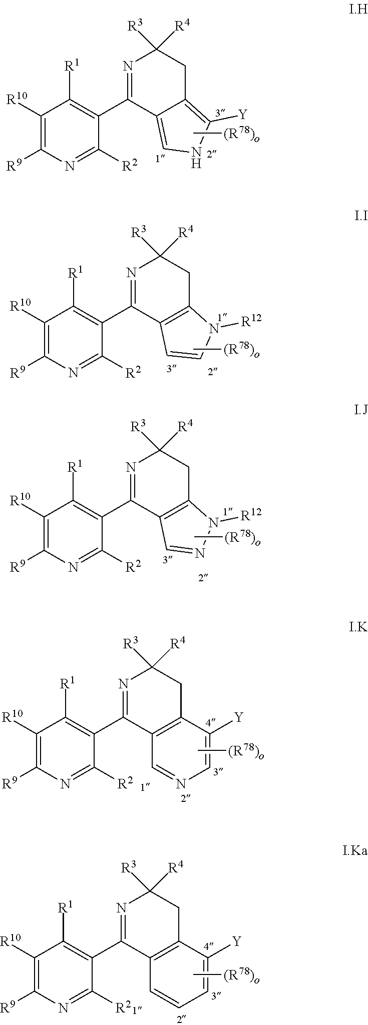

In still another embodiment of formula I, o in each of the formulae I.A, I.B, I.C, I.D, I.E, I.F, I.G, I.H, I.I, I.J I.K and I.Ka, respectively, is 0, i.e. the heteroaryl or phenyl group is not substituted.

These compounds are named I.A.1, I.B1, I.C.1, I.D.1, I.E.1, I.F.1, I.G.1, I.H.1, I.I.1, I.J.1 and I.K.1, I.Ka.1 respectively.

Further preferred compounds I are the following compounds I.L, I.M, I.N, I.O, I.P, I.Q, I.R, I.S, I.T and I.U. In these formulae, the substituents R¹, R², R³, R⁴, R⁵, R⁶, R⁹, R¹⁰, R⁷⁸ and o are independently as defined or preferably defined herein:

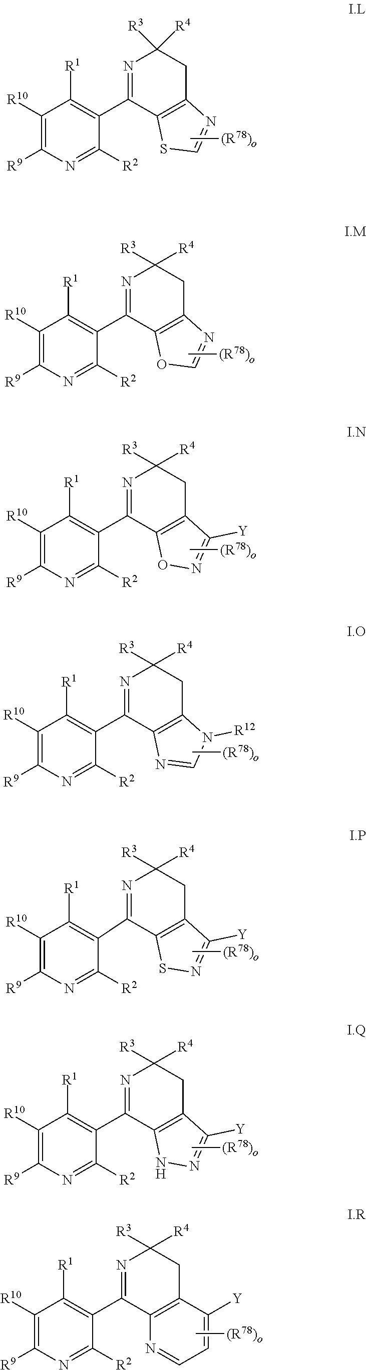

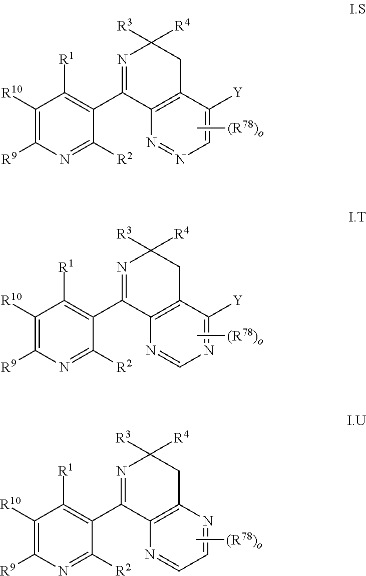

In still another embodiment of formula I,o in each of the formulae I.L, I.M, I.N, 1.0, I.P, I.Q, I.R, I.S, I.T and I.U, respectively, is 0, i.e. the heteroaryl is not substituted. These compounds are named I.L.1, I.M.1, I.N.1, 1.0.1, I.P.1, I.Q.1, I.R.1, I.S.1, I.T.1 and I.U.1, respectively.

In particular with a view to their use. In still another embodiment of formula I, preference is given to the compounds of the formulae I.A, I.B, I.C, I.D, I.E, I.F, I.G, I.H, I.I, I.J, I.K and I.Ka that are compiled in the Tables 1a to 80a, Tables 1b to 80b, Tables 1c to 15c, Tables 1d to 80d, Tables 1e to 80e, Tables 1f to 15f, Tables 1g to 90g, Tables 1h to 100h, Tables 1i to 90i, Tables 1j to 48j , Tables 1k to 150k, Tables 1Ka to 248ka. Each of the groups mentioned for a substituent in the tables is furthermore per se, independently of the combination in which it is mentioned, a particularly preferred aspect of the substituent in question.

Table 1a Compounds of formula I.A in which o is 0, Y is Br and the meaning for the combination of R¹, R², R³, R⁴, R⁹ and R¹⁰ for each individual compound corresponds in each case to one line of Table B.

Table 2a Compounds of formula I.A in which o is 1, R⁷⁸ is 2″-F, Y is Br and the meaning for the combination of R¹, R², R³, R⁴, R⁹ and R¹⁰ for each individual compound corresponds in each case to one line of Table B.

Table 3a Compounds of formula I.A in which o is 1, R⁷⁸ is 2″-Cl, Y is Br and the meaning for the combination of R¹, R², R³, R⁴, R⁹ and R¹⁰ for each individual compound corresponds in each case to one line of Table B.

Table 4a Compounds of formula I.A in which o is 1, R⁷⁸ is 2″-Br, Y is Br and the meaning for the combination of R¹, R², R³, R⁴, R⁹ and R¹⁰ for each individual compound corresponds in each case to one line of Table B.

Table 5a Compounds of formula I.A in which o is 1, R⁷⁸ is 2″-CH₃, Y is Br and the meaning for the combination of R¹, R², R³, R⁴, R⁹ and R¹⁰ for each individual compound corresponds in each case to one line of Table B.

Table 6a Compounds of formula I.A in which o is 1, R⁷⁸ is 2″-OCH₃, Y is Br and the meaning for the combination of R¹, R², R³, R⁴, R⁹ and R¹⁰ for each individual compound corresponds in each case to one line of Table B.

Table 7a Compounds of formula I.A in which o is 1, R⁷⁸ is 2″-OCHF₂, Y is Br and the meaning for the combination of R¹, R², R³, R⁴, R⁹ and R¹⁰ for each individual compound corresponds in each case to one line of Table B.

Table 8a Compounds of formula I.A in which o is 1, R⁷⁸ is 2″-C₆H₅, Y is Br and the meaning for the combination of R¹, R², R³, R⁴, R⁹ and R¹⁰ for each individual compound corresponds in each case to one line of Table B.

Table 9a Compounds of formula I.A in which o is 0, Y is CH₃ and the meaning for the combination of R¹, R², R³, R⁴, R⁹ and R¹⁰ for each individual compound corresponds in each case to one line of Table B.

Table 10a Compounds of formula I.A in which o is 1, R⁷⁸ is 2″-F, Y is CH₃ and the meaning for the combination of R¹, R², R³, R⁴, R⁹ and R¹⁰ for each individual compound corresponds in each case to one line of Table B.

Table 11a Compounds of formula I.A in which o is 1, R⁷⁸ is 2″-Cl, Y is CH₃ and the meaning for the combination of R¹, R², R³, R⁴, R⁹ and R¹⁰ for each individual compound corresponds in each case to one line of Table B.

Table 12a Compounds of formula I.A in which o is 1, R⁷⁸ is 2″-Br, Y is CH₃ and the meaning for the combination of R¹, R², R³, R⁴, R⁹ and R¹⁰ for each individual compound corresponds in each case to one line of Table B.

Table 13a Compounds of formula I.A in which o is 1, R⁷⁸ is 2″-CH₃, Y is CH₃ and the meaning for the combination of R¹, R², R³, R⁴, R⁹ and R¹⁰ for each individual compound corresponds in each case to one line of Table B.

Table 14a Compounds of formula I.A in which o is 1, R⁷⁸ is 2″-OCH₃, Y is CH₃ and the meaning for the combination of R¹, R², R³, R⁴, R⁹ and R¹⁰ for each individual compound corresponds in each case to one line of Table B.

Table 15a Compounds of formula I.A in which o is 1, R⁷⁸ is 2″-OCHF₂, Y is CH₃ and the meaning for the combination of R¹, R², R³, R⁴, R⁹ and R¹⁰ for each individual compound corresponds in each case to one line of Table B.

Table 16a Compounds of formula I.A in which o is 1, R⁷⁸ is 2″-C₆H₅, Y is CH₃ and the meaning for the combination of R¹, R², R³, R⁴, R⁹ and R¹⁰ for each individual compound corresponds in each case to one line of Table B.

Table 17a Compounds of formula I.A in which o is 0, Y is CF₃ and the meaning for the combination of R¹, R², R³, R⁴, R⁹ and R¹⁰ for each individual compound corresponds in each case to one line of Table B.

Table 18a Compounds of formula I.A in which o is 1, R⁷⁸ is 2″-F, Y is CF₃ and the meaning for the combination of R¹, R², R³, R⁴, R⁹ and R¹⁰ for each individual compound corresponds in each case to one line of Table B.

Table 19a Compounds of formula I.A in which o is 1, R⁷⁸ is 2″-Cl, Y is CF₃ and the meaning for the combination of R¹, R², R³, R⁴, R⁹ and R¹⁰ for each individual compound corresponds in each case to one line of Table B.

Table 20a Compounds of formula I.A in which o is 1, R⁷⁸ is 2″-Br, Y is CF₃ and the meaning for the combination of R¹, R², R³, R⁴, R⁹ and R¹⁰ for each individual compound corresponds in each case to one line of Table B.

Table 21a Compounds of formula I.A in which o is 1, R⁷⁸ is 2″-CH₃, Y is CF₃ and the meaning for the combination of R¹, R², R³, R⁴, R⁹ and R¹⁰ for each individual compound corresponds in each case to one line of Table B.

Table 22a Compounds of formula I.A in which o is 1, R⁷⁸ is 2″-OCH₃, Y is CF₃ and the meaning for the combination of R¹, R², R³, R⁴, R⁹ and R¹⁰ for each individual compound corresponds in each case to one line of Table B.

Table 23a Compounds of formula I.A in which o is 1, R⁷⁸ is 2″-OCHF₂, Y is CF₃ and the meaning for the combination of R¹, R², R³, R⁴, R⁹ and R¹⁰ for each individual compound corre-sponds in each case to one line of Table B.

Table 24a Compounds of formula I.A in which o is 1, R⁷⁸ is 2″-C₆H₅, Y is CF₃ and the mean-ing for the combination of R¹, R², R³, R⁴, R⁹ and R¹⁰ for each individual compound corresponds in each case to one line of Table B.

Table 25a Compounds of formula I.A in which o is 0, Y is CH(CH₃)₂ and the meaning for the combination of R¹, R², R³, R⁴, R⁹ and R¹⁰ for each individual compound corresponds in each case to one line of Table B.

Table 26a Compounds of formula I.A in which o is 1, R⁷⁸ is 2″-F, Y is CH(CH₃)₂ and the meaning for the combination of R¹, R², R³, R⁴, R⁹ and R¹⁰ for each individual compound corresponds in each case to one line of Table B.

Table 27a Compounds of formula I.A in which o is 1, R⁷⁸ is 2″-Cl, Y is CH(CH₃)₂ and the meaning for the combination of R¹, R², R³, R⁴, R⁹ and R¹⁰ for each individual compound corresponds in each case to one line of Table B.

Table 28a Compounds of formula I.A in which o is 1, R⁷⁸ is 2″-Br, Y is CH(CH₃)₂ and the meaning for the combination of R¹, R², R³, R⁴, R⁹ and R¹⁰ for each individual compound corresponds in each case to one line of Table B.

Table 29a Compounds of formula I.A in which o is 1, R⁷⁸ is 2″-CH₃, Y is CH(CH₃)₂ and the meaning for the combination of R¹, R², R³, R⁴, R⁹ and R¹⁰ for each individual compound corresponds in each case to one line of Table B.

Table 30a Compounds of formula I.A in which o is 1, R⁷⁸ is 2″-OCH₃, Y is CH(CH₃)₂ and the meaning for the combination of R¹, R², R³, R⁴, R⁹ and R¹⁰ for each individual compound corresponds in each case to one line of Table B.

Table 31a Compounds of formula I.A in which o is 1, R⁷⁸ is 2″-OCHF₂, Y is CH(CH₃)₂ and the meaning for the combination of R¹, R², R³, R⁴, R⁹ and R¹⁰ for each individual compound corresponds in each case to one line of Table B.

Table 32a Compounds of formula I.A in which o is 1, R⁷⁸ is 2″-C₆H₅, Y is CH(CH₃)₂ and the meaning for the combination of R¹, R², R³, R⁴, R⁹ and R¹⁰ for each individual compound corresponds in each case to one line of Table B.

Table 33a Compounds of formula I.A in which o is 0, Y is C₂H₅ and the meaning for the combination of R¹, R², R³, R⁴, R⁹ and R¹⁰ for each individual compound corresponds in each case to one line of Table B.

Table 34a Compounds of formula I.A in which o is 1, R⁷⁸ is 2″-F, Y is C₂H₅ and the meaning for the combination of R¹, R², R³, R⁴, R⁹ and R¹⁰ for each individual compound corresponds in each case to one line of Table B.

Table 35a Compounds of formula I.A in which o is 1, R⁷⁸ is 2″-Cl, Y is C₂H₅ and the meaning for the combination of R¹, R², R³, R⁴, R⁹ and R¹⁰ for each individual compound corresponds in each case to one line of Table B.

Table 36a Compounds of formula I.A in which o is 1, R⁷⁸ is 2″-Br, Y is C₂H₅ and the meaning for the combination of R¹, R², R³, R⁴, R⁹ and R¹⁰ for each individual compound corresponds in each case to one line of Table B.

Table 37a Compounds of formula I.A in which o is 1, R⁷⁸ is 2″-CH₃, Y is C₂H₅ and the meaning for the combination of R¹, R², R³, R⁴, R⁹ and R¹⁰ for each individual compound corresponds in each case to one line of Table B.

Table 38a Compounds of formula I.A in which o is 1, R⁷⁸ is 2″-OCH₃, Y is C₂H₅ and the meaning for the combination of R¹, R², R³, R⁴, R⁹ and R¹⁰ for each individual compound corresponds in each case to one line of Table B.

Table 39a Compounds of formula I.A in which o is 1, R⁷⁸ is 2″-OCHF₂, Y is C₂H₅ and the meaning for the combination of R¹, R², R³, R⁴, R⁹ and R¹⁰ for each individual compound corresponds in each case to one line of Table B.

Table 40a Compounds of formula I.A in which o is 1, R⁷⁸ is 2″-C₆H₅, Y is C₂H₅ and the meaning for the combination of R¹, R², R³, R⁴, R⁹ and R¹⁰ for each individual compound corresponds in each case to one line of Table B.

Table 41a Compounds of formula I.A in which o is 0, Y is CN and the meaning for the combination of R¹, R², R³, R⁴, R⁹ and R¹⁰ for each individual compound corresponds in each case to one line of Table B.

Table 42a Compounds of formula I.A in which o is 1, R⁷⁸ is 2″-F, Y is CN and the meaning for the combination of R¹, R², R³, R⁴, R⁹ and R¹⁰ for each individual compound corresponds in each case to one line of Table B.

Table 43a Compounds of formula I.A in which o is 1, R⁷⁸ is 2″-Cl, Y is CN and the meaning for the combination of R¹, R², R³, R⁴, R⁹ and R¹⁰ for each individual compound corresponds in each case to one line of Table B.

Table 44a Compounds of formula I.A in which o is 1, R⁷⁸ is 2″-Br, Y is CN and the meaning for the combination of R¹, R², R³, R⁴, R⁹ and R¹⁰ for each individual compound corresponds in each case to one line of Table B.

Table 45a Compounds of formula I.A in which o is 1, R⁷⁸ is 2″-CH₃, Y is CN and the meaning for the combination of R¹, R², R³, R⁴, R⁹ and R¹⁰ for each individual compound corresponds in each case to one line of Table B.

Table 46a Compounds of formula I.A in which o is 1, R⁷⁸ is 2″-OCH₃, Y is CN and the meaning for the combination of R¹, R², R³, R⁴, R⁹ and R¹⁰ for each individual compound corresponds in each case to one line of Table B.

Table 47a Compounds of formula I.A in which o is 1, R⁷⁸ is 2″-OCHF₂, Y is CN and the meaning for the combination of R¹, R², R³, R⁴, R⁹ and R¹⁰ for each individual compound corresponds in each case to one line of Table B.

Table 48a Compounds of formula I.A in which o is 1, R⁷⁸ is 2″-C₆H₅, Y is CN and the meaning for the combination of R¹, R², R³, R⁴, R⁹ and R¹⁰ for each individual compound corresponds in each case to one line of Table B.

Table 49a Compounds of formula I.A in which o is 0, Y is CHF₂ and the meaning for the combination of R¹, R², R³, R⁴, R⁹ and R¹⁰ for each individual compound corresponds in each case to one line of Table B.

Table 50a Compounds of formula I.A in which o is 1, R⁷⁸ is 2″-F, Y is CHF₂ and the meaning for the combination of R¹, R², R³, R⁴, R⁹ and R¹⁰ for each individual compound corresponds in each case to one line of Table B.

Table 51a Compounds of formula I.A in which o is 1, R⁷⁸ is 2″-Cl, Y is CHF₂ and the meaning for the combination of R¹, R², R³, R⁴, R⁹ and R¹⁰ for each individual compound corresponds in each case to one line of Table B.

Table 52a Compounds of formula I.A in which o is 1, R⁷⁸ is 2″-Br, Y is CHF₂ and the meaning for the combination of R¹, R², R³, R⁴, R⁹ and R¹⁰ for each individual compound corresponds in each case to one line of Table B.

Table 53a Compounds of formula I.A in which o is 1, R⁷⁸ is 2″-CH₃, Y is CHF₂ and the meaning for the combination of R¹, R², R³, R⁴, R⁹ and R¹⁰ for each individual compound corresponds in each case to one line of Table B.

Table 54a Compounds of formula I.A in which o is 1, R⁷⁸ is 2″-OCH₃, Y is CHF₂ and the meaning for the combination of R¹, R², R³, R⁴, R⁹ and R¹⁰ for each individual compound corresponds in each case to one line of Table B.

Table 55a Compounds of formula I.A in which o is 1, R⁷⁸ is 2″-OCHF₂, Y is CHF₂ and the meaning for the combination of R¹, R², R³, R⁴, R⁹ and R¹⁰ for each individual compound corresponds in each case to one line of Table B.

Table 56a Compounds of formula I.A in which o is 1, R⁷⁸ is 2″-C₆H₅, Y is CHF₂ and the meaning for the combination of R¹, R², R³, R⁴, R⁹ and R¹⁰ for each individual compound corresponds in each case to one line of Table B.

Table 57a Compounds of formula I.A in which o is 0, Y is OCHF₂ and the meaning for the combination of R¹, R², R³, R⁴, R⁹ and R¹⁰ for each individual compound corresponds in each case to one line of Table B.

Table 58a Compounds of formula I.A in which o is 1, R⁷⁸ is 2″-F, Y is OCHF₂ and the meaning for the combination of R¹, R², R³, R⁴, R⁹ and R¹⁰ for each individual compound corresponds in each case to one line of Table B.

Table 59a Compounds of formula I.A in which o is 1, R⁷⁸ is 2″-Cl, Y is OCHF₂ and the meaning for the combination of R¹, R², R³, R⁴, R⁹ and R¹⁰ for each individual compound corresponds in each case to one line of Table B.

Table 60a Compounds of formula I.A in which o is 1, R⁷⁸ is 2″-Br, Y is OCHF₂ and the meaning for the combination of R¹, R², R³, R⁴, R⁹ and R¹⁰ for each individual compound corresponds in each case to one line of Table B.

Table 61a Compounds of formula I.A in which o is 1, R⁷⁸ is 2″-CH₃, Y is OCHF₂ and the meaning for the combination of R¹, R², R³, R⁴, R⁹ and R¹⁰ for each individual compound corresponds in each case to one line of Table B.

Table 62a Compounds of formula I.A in which o is 1, R⁷⁸ is 2″-OCH₃, Y is OCHF₂ and the meaning for the combination of R¹, R², R³, R⁴, R⁹ and R¹⁰ for each individual compound corresponds in each case to one line of Table B.

Table 63a Compounds of formula I.A in which o is 1, R⁷⁸ is 2″-OCHF₂, Y is OCHF₂ and the meaning for the combination of R¹, R², R³, R⁴, R⁹ and R¹⁰ for each individual compound corresponds in each case to one line of Table B.

Table 64a Compounds of formula I.A in which o is 1, R⁷⁸ is 2″-C₆H₅, Y is OCHF₂ and the meaning for the combination of R¹, R², R³, R⁴, R⁹ and R¹⁰ for each individual compound corresponds in each case to one line of Table B.

Table 65a Compounds of formula I.A in which o is 0, Y is Cl and the meaning for the combination of R¹, R², R³, R⁴, R⁹ and R¹⁰ for each individual compound corresponds in each case to one line of Table B.

Table 66a Compounds of formula I.A in which o is 1, R⁷⁸ is 2″-F, Y is Cl and the meaning for the combination of R¹, R², R³, R⁴, R⁹ and R¹⁰ for each individual compound corresponds in each case to one line of Table B.

Table 67a Compounds of formula I.A in which o is 1, R⁷⁸ is 2″-Cl, Y is Cl and the meaning for the combination of R¹, R², R³, R⁴, R⁹ and R¹⁰ for each individual compound corresponds in each case to one line of Table B.

Table 68a Compounds of formula I.A in which o is 1, R⁷⁸ is 2″-Br, Y is Cl and the meaning for the combination of R¹, R², R³, R⁴, R⁹ and R¹⁰ for each individual compound corresponds in each case to one line of Table B.

Table 69a Compounds of formula I.A in which o is 1, R⁷⁸ is 2″-CH₃, Y is Cl and the meaning for the combination of R¹, R², R³, R⁴, R⁹ and R¹⁰ for each individual compound corresponds in each case to one line of Table B.

Table 70a Compounds of formula I.A in which o is 1, R⁷⁸ is 2″-OCH₃, Y is Cl and the meaning for the combination of R¹, R², R³, R⁴, R⁹ and R¹⁰ for each individual compound corresponds in each case to one line of Table B.

Table 71a Compounds of formula I.A in which o is 1, R⁷⁸ is 2″-OCHF₂, Y is Cl and the meaning for the combination of R¹, R², R³, R⁴, R⁹ and R¹⁰ for each individual compound corresponds in each case to one line of Table B.

Table 72a Compounds of formula I.A in which o is 1, R⁷⁸ is 2″-C₆H₅, Y is Cl and the meaning for the combination of R¹, R², R³, R⁴, R⁹ and R¹⁰ for each individual compound corresponds in each case to one line of Table B.

Table 73a Compounds of formula I.A in which o is 0, Y is F and the meaning for the combination of R¹, R², R³, R⁴, R⁹ and R¹⁰ for each individual compound corresponds in each case to one line of Table B.

Table 74a Compounds of formula I.A in which o is 1, R⁷⁸ is 2″-F, Y is F and the meaning for the combination of R¹, R², R³, R⁴, R⁹ and R¹⁰ for each individual compound corresponds in each case to one line of Table B.

Table 75a Compounds of formula I.A in which o is 1, R⁷⁸ is 2″-Cl, Y is F and the meaning for the combination of R¹, R², R³, R⁴, R⁹ and R¹⁰ for each individual compound corresponds in each case to one line of Table B.

Table 76a Compounds of formula I.A in which o is 1, R⁷⁸ is 2″-Br, Y is F and the meaning for the combination of R¹, R², R³, R⁴, R⁹ and R¹⁰ for each individual compound corresponds in each case to one line of Table B.

Table 77a Compounds of formula I.A in which o is 1, R⁷⁸ is 2″-CH₃, Y is F and the meaning for the combination of R¹, R², R³, R⁴, R⁹ and R¹⁰ for each individual compound corresponds in each case to one line of Table B.

Table 78a Compounds of formula I.A in which o is 1, R⁷⁸ is 2″-OCH₃, Y is F and the meaning for the combination of R¹, R², R³, R⁴, R⁹ and R¹⁰ for each individual compound corresponds in each case to one line of Table B.

Table 79a Compounds of formula I.A in which o is 1, R⁷⁸ is 2″-OCHF₂, Y is F and the meaning for the combination of R¹, R², R³, R⁴, R⁹ and R¹⁰ for each individual compound corresponds in each case to one line of Table B.

Table 80a Compounds of formula I.A in which o is 1, R⁷⁸ is 2″-C₆H₅, Y is F and the meaning for the combination of R¹, R², R³, R⁴, R⁹ and R¹⁰ for each individual compound corresponds in each case to one line of Table B.

Table 1 b Compounds of formula I.B in which o is 0, Y is Br and the meaning for the combination of R¹, R², R³, R⁴, R⁹ and R¹⁰ for each individual compound corresponds in each case to one line of Table B.

Table 2b Compounds of formula I.B in which o is 1, R⁷⁸ is 1″-F, Y is Br and the meaning for the combination of R¹, R², R³, R⁴, R⁹ and R¹⁰ for each individual compound corresponds in each case to one line of Table B.

Table 3b Compounds of formula I.B in which o is 1, R⁷⁸ is 1″-Cl, Y is Br and the meaning for the combination of R¹, R², R³, R⁴, R⁹ and R¹⁰ for each individual compound corresponds in each case to one line of Table B.

Table 4b Compounds of formula I.B in which o is 1, R⁷⁸ is 1″-Br, Y is Br and the meaning for the combination of R¹, R², R³, R⁴, R⁹ and R¹⁰ for each individual compound corresponds in each case to one line of Table B.

Table 5b Compounds of formula I.B in which o is 1, R⁷⁸ is 1″-CH₃, Y is Br and the meaning for the combination of R¹, R², R³, R⁴, R⁹ and R¹⁰ for each individual compound corresponds in each case to one line of Table B.

Table 6b Compounds of formula I.B in which o is 1, R⁷⁸ is 1″-OCH₃, Y is Br and the meaning for the combination of R¹, R², R³, R⁴, R⁹ and R¹⁰ for each individual compound corresponds in each case to one line of Table B.

Table 7b Compounds of formula I.B in which o is 1, R⁷⁸ is 1″-OCHF₂, Y is Br and the meaning for the combination of R¹, R², R³, R⁴, R⁹ and R¹⁰ for each individual compound corresponds in each case to one line of Table B.

Table 8b Compounds of formula I.B in which o is 1, R⁷⁸ is 1″-C₆H₅, Y is Br and the meaning for the combination of R¹, R², R³, R⁴, R⁹ and R¹⁰ for each individual compound corresponds in each case to one line of Table B.

Table 9b Compounds of formula I.B in which o is 0, Y is CH₃ and the meaning for the combination of R¹, R², R³, R⁴, R⁹ and R¹⁰ for each individual compound corresponds in each case to one line of Table B.

Table 10b Compounds of formula I.B in which o is 1, R⁷⁸ is 1″-F, Y is CH₃ and the meaning for the combination of R¹, R², R³, R⁴, R⁹ and R¹⁰ for each individual compound corresponds in each case to one line of Table B.

Table 11b Compounds of formula I.B in which o is 1, R⁷⁸ is 1″-Cl, Y is CH₃ and the meaning for the combination of R¹, R², R³, R⁴, R⁹ and R¹⁰ for each individual compound corresponds in each case to one line of Table B.

Table 12b Compounds of formula I.B in which o is 1, R⁷⁸ is 1″-Br, Y is CH₃ and the meaning for the combination of R¹, R², R³, R⁴, R⁹ and R¹⁰ for each individual compound corresponds in each case to one line of Table B.

Table 13b Compounds of formula I.B in which o is 1, R⁷⁸ is 1″-CH₃, Y is CH₃ and the meaning for the combination of R¹, R², R³, R⁴, R⁹ and R¹⁰ for each individual compound corresponds in each case to one line of Table B.

Table 14b Compounds of formula I.B in which o is 1, R⁷⁸ is 1″-OCH₃, Y is CH₃ and the meaning for the combination of R¹, R², R³, R⁴, R⁹ and R¹⁰ for each individual compound corresponds in each case to one line of Table B.

Table 15b Compounds of formula I.B in which o is 1, R⁷⁸ is 1″-OCHF₂, Y is CH₃ and the meaning for the combination of R¹, R², R³, R⁴, R⁹ and R¹⁰ for each individual compound corresponds in each case to one line of Table B.

Table 16b Compounds of formula I.B in which o is 1, R⁷⁸ is 1″-C₆H₅, Y is CH₃ and the meaning for the combination of R¹, R², R³, R⁴, R⁹ and R¹⁰ for each individual compound corresponds in each case to one line of Table B.

Table 17b Compounds of formula I.B in which o is 0, Y is CF₃ and the meaning for the combination of R¹, R², R³, R⁴, R⁹ and R¹⁰ for each individual compound corresponds in each case to one line of Table B.

Table 18b Compounds of formula I.B in which o is 1, R⁷⁸ is 1″-F, Y is CF₃ and the meaning for the combination of R¹, R², R³, R⁴, R⁹ and R¹⁰ for each individual compound corresponds in each case to one line of Table B.

Table 19b Compounds of formula I.B in which o is 1, R⁷⁸ is 1″-Cl, Y is CF₃ and the meaning for the combination of R¹, R², R³, R⁴, R⁹ and R¹⁰ for each individual compound corresponds in each case to one line of Table B.

Table 20b Compounds of formula I.B in which o is 1, R⁷⁸ is 1″-Br, Y is CF₃ and the meaning for the combination of R¹, R², R³, R⁴, R⁹ and R¹⁰ for each individual compound corresponds in each case to one line of Table B.

Table 21b Compounds of formula I.B in which o is 1, R⁷⁸ is 1″-CH₃, Y is CF₃ and the meaning for the combination of R¹, R², R³, R⁴, R⁹ and R¹⁰ for each individual compound corresponds in each case to one line of Table B.

Table 22b Compounds of formula I.B in which o is 1, R⁷⁸ is 1″-OCH₃, Y is CF₃ and the meaning for the combination of R¹, R², R³, R⁴, R⁹ and R¹⁰ for each individual compound corresponds in each case to one line of Table B.

Table 23b Compounds of formula I.B in which o is 1, R⁷⁸ is 1″-OCHF₂, Y is CF₃ and the meaning for the combination of R¹, R², R³, R⁴, R⁹ and R¹⁰ for each individual compound corresponds in each case to one line of Table B.

Table 24b Compounds of formula I.B in which o is 1, R⁷⁸ is 1″-C₆H₅, Y is CF₃ and the meaning for the combination of R¹, R², R³, R⁴, R⁹ and R¹⁰ for each individual compound corresponds in each case to one line of Table B.

Table 25b Compounds of formula I.B in which o is 0, Y is CH(CH₃)₂ and the meaning for the combination of R¹, R², R³, R⁴, R⁹ and R¹⁰ for each individual compound corresponds in each case to one line of Table B.

Table 26b Compounds of formula I.B in which o is 1, R⁷⁸ is 1″-F, Y is CH(CH₃)₂ and the meaning for the combination of R¹, R², R³, R⁴, R⁹ and R¹⁰ for each individual compound corresponds in each case to one line of Table B.

Table 27b Compounds of formula I.B in which o is 1, R⁷⁸ is 1″-Cl, Y is CH(CH₃)₂ and the meaning for the combination of R¹, R², R³, R⁴, R⁹ and R¹⁰ for each individual compound corresponds in each case to one line of Table B.

Table 28b Compounds of formula I.B in which o is 1, R⁷⁸ is 1″-Br, Y is CH(CH₃)₂ and the meaning for the combination of R¹, R², R³, R⁴, R⁹ and R¹⁰ for each individual compound corresponds in each case to one line of Table B.

Table 29b Compounds of formula I.B in which o is 1, R⁷⁸ is 1″-CH₃, Y is CH(CH₃)₂ and the meaning for the combination of R¹, R², R³, R⁴, R⁹ and R¹⁰ for each individual compound corresponds in each case to one line of Table B.

Table 30b Compounds of formula I.B in which o is 1, R⁷⁸ is 1″-OCH₃, Y is CH(CH₃)₂ and the meaning for the combination of R¹, R², R³, R⁴, R⁹ and R¹⁰ for each individual compound corresponds in each case to one line of Table B.

Table 31b Compounds of formula I.B in which o is 1, R⁷⁸ is 1″-OCHF₂, Y is CH(CH₃)₂ and the meaning for the combination of R¹, R², R³, R⁴, R⁹ and R¹⁰ for each individual compound corresponds in each case to one line of Table B.

Table 32b Compounds of formula I.B in which o is 1, R⁷⁸ is 1″-C₆H₅, Y is CH(CH₃)₂ and the meaning for the combination of R¹, R², R³, R⁴, R⁹ and R¹⁰ for each individual compound corresponds in each case to one line of Table B.

Table 33b Compounds of formula I.B in which o is 0, Y is C₂H₅ and the meaning for the combination of R¹, R², R³, R⁴, R⁹ and R¹⁰ for each individual compound corresponds in each case to one line of Table B.

Table 34b Compounds of formula I.B in which o is 1, R⁷⁸ is 1″-F, Y is C₂H₅ and the meaning for the combination of R¹, R², R³, R⁴, R⁹ and R¹⁰ for each individual compound corresponds in each case to one line of Table B.

Table 35b Compounds of formula I.B in which o is 1, R⁷⁸ is 1″-Cl, Y is C₂H₅ and the meaning for the combination of R¹, R², R³, R⁴, R⁹ and R¹⁰ for each individual compound corresponds in each case to one line of Table B.

Table 36b Compounds of formula I.B in which o is 1, R⁷⁸ is 1″-Br, Y is C₂H₅ and the meaning for the combination of R¹, R², R³, R⁴, R⁹ and R¹⁰ for each individual compound corresponds in each case to one line of Table B.

Table 37b Compounds of formula I.B in which o is 1, R⁷⁸ is 1″-CH₃, Y is C₂H₅ and the meaning for the combination of R¹, R², R³, R⁴, R⁹ and R¹⁰ for each individual compound corresponds in each case to one line of Table B.

Table 38b Compounds of formula I.B in which o is 1, R⁷⁸ is 1″-OCH₃, Y is C₂H₅ and the meaning for the combination of R¹, R², R³, R⁴, R⁹ and R¹⁰ for each individual compound corresponds in each case to one line of Table B.

Table 39b Compounds of formula I.B in which o is 1, R⁷⁸ is 1″-OCHF₂, Y is C₂H₅ and the meaning for the combination of R¹, R², R³, R⁴, R⁹ and R¹⁰ for each individual compound corresponds in each case to one line of Table B.

Table 40b Compounds of formula I.B in which o is 1, R⁷⁸ is 1″-C₆H₅, Y is C₂H₅ and the meaning for the combination of R¹, R², R³, R⁴, R⁹ and R¹⁰ for each individual compound corresponds in each case to one line of Table B.

Table 41b Compounds of formula I.B in which o is 0, Y is CHF₂ and the meaning for the combination of R¹, R², R³, R⁴, R⁹ and R¹⁰ for each individual compound corresponds in each case to one line of Table B.

Table 42b Compounds of formula I.B in which o is 1, R⁷⁸ is 1″-F, Y is CHF₂ and the meaning for the combination of R¹, R², R³, R⁴, R⁹ and R¹⁰ for each individual compound corresponds in each case to one line of Table B.

Table 43b Compounds of formula I.B in which o is 1, R⁷⁸ is 1″-Cl, Y is CHF₂ and the meaning for the combination of R¹, R², R³, R⁴, R⁹ and R¹⁰ for each individual compound corresponds in each case to one line of Table B.

Table 44b Compounds of formula I.B in which o is 1, R⁷⁸ is 1″-Br, Y is CHF₂ and the meaning for the combination of R¹, R², R³, R⁴, R⁹ and R¹⁰ for each individual compound corresponds in each case to one line of Table B.

Table 45b Compounds of formula I.B in which o is 1, R⁷⁸ is 1″-CH₃, Y is CHF₂ and the meaning for the combination of R¹, R², R³, R⁴, R⁹ and R¹⁰ for each individual compound corresponds in each case to one line of Table B.

Table 46b Compounds of formula I.B in which o is 1, R⁷⁸ is 1″-OCH₃, Y is CHF₂ and the meaning for the combination of R¹, R², R³, R⁴, R⁹ and R¹⁰ for each individual compound corresponds in each case to one line of Table B.

Table 47b Compounds of formula I.B in which o is 1, R⁷⁸ is 1″-OCHF₂, Y is CHF₂ and the meaning for the combination of R¹, R², R³, R⁴, R⁹ and R¹⁰ for each individual compound corresponds in each case to one line of Table B.

Table 48b Compounds of formula I.B in which o is 1, R⁷⁸ is 1″-C₆H₅, Y is CHF₂ and the meaning for the combination of R¹, R², R³, R⁴, R⁹ and R¹⁰ for each individual compound corresponds in each case to one line of Table B.

Table 49b Compounds of formula I.B in which o is 0, Y is CN and the meaning for the combination of R¹, R², R³, R⁴, R⁹ and R¹⁰ for each individual compound corresponds in each case to one line of Table B.

Table 50b Compounds of formula I.B in which o is 1, R⁷⁸ is 1″-F, Y is CN and the meaning for the combination of R¹, R², R³, R⁴, R⁹ and R¹⁰ for each individual compound corresponds in each case to one line of Table B.

Table 51b Compounds of formula I.B in which o is 1, R⁷⁸ is 1″-Cl, Y is CN and the meaning for the combination of R¹, R², R³, R⁴, R⁹ and R¹⁰ for each individual compound corresponds in each case to one line of Table B.

Table 52b Compounds of formula I.B in which o is 1, R⁷⁸ is 1″-Br, Y is CN and the meaning for the combination of R¹, R², R³, R⁴, R⁹ and R¹⁰ for each individual compound corresponds in each case to one line of Table B.

Table 53b Compounds of formula I.B in which o is 1, R⁷⁸ is 1″-CH₃, Y is CN and the meaning for the combination of R¹, R², R³, R⁴, R⁹ and R¹⁰ for each individual compound corresponds in each case to one line of Table B.

Table 54b Compounds of formula I.B in which o is 1, R⁷⁸ is 1″-OCH₃, Y is CN and the meaning for the combination of R¹, R², R³, R⁴, R⁹ and R¹⁰ for each individual compound corresponds in each case to one line of Table B.

Table 55b Compounds of formula I.B in which o is 1, R⁷⁸ is 1″-OCHF₂, Y is CN and the meaning for the combination of R¹, R², R³, R⁴, R⁹ and R¹⁰ for each individual compound corresponds in each case to one line of Table B.

Table 56b Compounds of formula I.B in which o is 1, R⁷⁸ is 1″-C₆H₅, Y is CN and the meaning for the combination of R¹, R², R³, R⁴, R⁹ and R¹⁰ for each individual compound corresponds in each case to one line of Table B.

Table 57b Compounds of formula I.B in which o is 0, Y is OCHF₂ and the meaning for the combination of R¹, R², R³, R⁴, R⁹ and R¹⁰ for each individual compound corresponds in each case to one line of Table B.

Table 58b Compounds of formula I.B in which o is 1, R⁷⁸ is 1″-F, Y is OCHF₂ and the meaning for the combination of R¹, R², R³, R⁴, R⁹ and R¹⁰ for each individual compound corresponds in each case to one line of Table B.

Table 59b Compounds of formula I.B in which o is 1, R⁷⁸ is 1″-Cl, Y is OCHF₂ and the meaning for the combination of R¹, R², R³, R⁴, R⁹ and R¹⁰ for each individual compound corresponds in each case to one line of Table B.

Table 60b Compounds of formula I.B in which o is 1, R⁷⁸ is 1″-Br, Y is OCHF₂ and the meaning for the combination of R¹, R², R³, R⁴, R⁹ and R¹⁰ for each individual compound corresponds in each case to one line of Table B.

Table 61b Compounds of formula I.B in which o is 1, R⁷⁸ is 1″-CH₃, Y is OCHF₂ and the meaning for the combination of R¹, R², R³, R⁴, R⁹ and R¹⁰ for each individual compound corresponds in each case to one line of Table B.

Table 62b Compounds of formula I.B in which o is 1, R⁷⁸ is 1″-OCH₃, Y is OCHF₂ and the meaning for the combination of R¹, R², R³, R⁴, R⁹ and R¹⁰ for each individual compound corresponds in each case to one line of Table B.

Table 63b Compounds of formula I.B in which o is 1, R⁷⁸ is 1″-OCHF₂, Y is OCHF₂ and the meaning for the combination of R¹, R², R³, R⁴, R⁹ and R¹⁰ for each individual compound corresponds in each case to one line of Table B.

Table 64b Compounds of formula I.B in which o is 1, R⁷⁸ is 1″-C₆H₅, Y is OCHF₂ and the meaning for the combination of R¹, R², R³, R⁴, R⁹ and R¹⁰ for each individual compound corresponds in each case to one line of Table B.

Table 65b Compounds of formula I.B in which o is 0, Y is Cl and the meaning for the combination of R¹, R², R³, R⁴, R⁹ and R¹⁰ for each individual compound corresponds in each case to one line of Table B.

Table 66b Compounds of formula I.B in which o is 1, R⁷⁸ is 1″-F, Y is Cl and the meaning for the combination of R¹, R², R³, R⁴, R⁹ and R¹⁰ for each individual compound corresponds in each case to one line of Table B.

Table 67b Compounds of formula I.B in which o is 1, R⁷⁸ is 1″-Cl, Y is Cl and the meaning for the combination of R¹, R², R³, R⁴, R⁹ and R¹⁰ for each individual compound corresponds in each case to one line of Table B.

Table 68b Compounds of formula I.B in which o is 1, R⁷⁸ is 1″-Br, Y is Cl and the meaning for the combination of R¹, R², R³, R⁴, R⁹ and R¹⁰ for each individual compound corresponds in each case to one line of Table B.

Table 69b Compounds of formula I.B in which o is 1, R⁷⁸ is 1″-CH₃, Y is Cl and the meaning for the combination of R¹, R², R³, R⁴, R⁹ and R¹⁰ for each individual compound corresponds in each case to one line of Table B.

Table 70b Compounds of formula I.B in which o is 1, R⁷⁸ is 1″-OCH₃, Y is Cl and the meaning for the combination of R¹, R², R³, R⁴, R⁹ and R¹⁰ for each individual compound corresponds in each case to one line of Table B.

Table 71b Compounds of formula I.B in which o is 1, R⁷⁸ is 1″-OCHF₂, Y is Cl and the meaning for the combination of R¹, R², R³, R⁴, R⁹ and R¹⁰ for each individual compound corresponds in each case to one line of Table B.

Table 72b Compounds of formula I.B in which o is 1, R⁷⁸ is 1″-C₆H₅, Y is Cl and the meaning for the combination of R¹, R², R³, R⁴, R⁹ and R¹⁰ for each individual compound corresponds in each case to one line of Table B.

Table 73b Compounds of formula I.B in which o is 0, Y is F and the meaning for the combination of R¹, R², R³, R⁴, R⁹ and R¹⁰ for each individual compound corresponds in each case to one line of Table B.

Table 74b Compounds of formula I.B in which o is 1, R⁷⁸ is 1″-F, Y is F and the meaning for the combination of R¹, R², R³, R⁴, R⁹ and R¹⁰ for each individual compound corresponds in each case to one line of Table B.

Table 75b Compounds of formula I.B in which o is 1, R⁷⁸ is 1″-Cl, Y is F and the meaning for the combination of R¹, R², R³, R⁴, R⁹ and R¹⁰ for each individual compound corresponds in each case to one line of Table B.

Table 76b Compounds of formula I.B in which o is 1, R⁷⁸ is 1″-Br, Y is F and the meaning for the combination of R¹, R², R³, R⁴, R⁹ and R¹⁰ for each individual compound corresponds in each case to one line of Table B.

Table 77b Compounds of formula I.B in which o is 1, R⁷⁸ is 1″-CH₃, Y is F and the meaning for the combination of R¹, R², R³, R⁴, R⁹ and R¹⁰ for each individual compound corresponds in each case to one line of Table B.

Table 78b Compounds of formula I.B in which o is 1, R⁷⁸ is 1″-OCH₃, Y is F and the meaning for the combination of R¹, R², R³, R⁴, R⁹ and R¹⁰ for each individual compound corresponds in each case to one line of Table B.

Table 79b Compounds of formula I.B in which o is 1, R⁷⁸ is 1″-OCHF₂, Y is F and the meaning for the combination of R¹, R², R³, R⁴, R⁹ and R¹⁰ for each individual compound corresponds in each case to one line of Table B.

Table 80b Compounds of formula I.B in which o is 1, R⁷⁸ is 1″-C₆H₅, Y is F and the meaning for the combination of R¹, R², R³, R⁴, R⁹ and R¹⁰ for each individual compound corresponds in each case to one line of Table B.

Table 1c Compounds of formula I.C in which o is 0, and the meaning for the combination of R¹, R², R³, R⁴, R⁹ and R¹⁰ for each individual compound corresponds in each case to one line of Table B.

Table 2c Compounds of formula I.C in which o is 1, R⁷⁸ is 2″-F and the meaning for the combination of R¹, R², R³, R⁴, R⁹ and R¹⁰ for each individual compound corresponds in each case to one line of Table B.

Table 3c Compounds of formula I.C in which o is 1, R⁷⁸ is 2″-Cl and the meaning for the combination of R¹, R², R³, R⁴, R⁹ and R¹⁰ for each individual compound corresponds in each case to one line of Table B.

Table 4c Compounds of formula I.C in which o is 1, R⁷⁸ is 2″-Br and the meaning for the combination of R¹, R², R³, R⁴, R⁹ and R¹⁰ for each individual compound corresponds in each case to one line of Table B.

Table 5c Compounds of formula I.C in which o is 1, R⁷⁸ is 2″-CH₃ and the meaning for the combination of R¹, R², R³, R⁴, R⁹ and R¹⁰ for each individual compound corresponds in each case to one line of Table B.

Table 6c Compounds of formula I.C in which o is 1, R⁷⁸ is 2″-OCH₃ and the meaning for the combination of R¹, R², R³, R⁴, R⁹ and R¹⁰ for each individual compound corresponds in each case to one line of Table B.

Table 7c Compounds of formula I.C in which o is 1, R⁷⁸ is 2″-OCHF₂ and the meaning for the combination of R¹, R², R³, R⁴, R⁵ R⁶, R⁹ and R¹⁰ for each individual compound corresponds in each case to one line of Table B.

Table 8c Compounds of formula I.C in which o is 1, R⁷⁸ is 2″-C₆H₅ and the meaning for the combination of R¹, R², R³, R⁴, R⁹ and R¹⁰ for each individual compound corresponds in each case to one line of Table B.

Table 9c Compounds of formula I.C in which o is 1, R⁷⁸ is 3″-F and the meaning for the combination of R¹, R², R³, R⁴, R⁹ and R¹⁰ for each individual compound corresponds in each case to one line of Table B.

Table 10c Compounds of formula I.C in which o is 1, R⁷⁸ is 3″-Cl and the meaning for the combination of R¹, R², R³, R⁴, R⁹ and R¹⁰ for each individual compound corresponds in each case to one line of Table B.

Table 11c Compounds of formula I.C in which o is 1, R⁷⁸ is 3″-Br and the meaning for the combination of R¹, R², R³, R⁴, R⁹ and R¹⁰ for each individual compound corresponds in each case to one line of Table B.

Table 12c Compounds of formula I.C in which o is 1, R⁷⁸ is 3″-CH₃ F and the meaning for the combination of R¹, R², R³, R⁴, R⁹ and R¹⁰ for each individual compound corresponds in each case to one line of Table B.

Table 13c Compounds of formula I.C in which o is 1, R⁷⁸ is 3″-OCH₃ and the meaning for the combination of R¹, R², R³, R⁴, R⁹ and R¹⁰ for each individual compound corresponds in each case to one line of Table B.

Table 14c Compounds of formula I.C in which o is 1, R⁷⁸ is 3″-OCHF₂ and the meaning for the combination of R¹, R², R³, R⁴, R⁹ and R¹⁰ for each individual compound corresponds in each case to one line of Table B.

Table 15c Compounds of formula I.C in which o is 1, R⁷⁸ is 3″-C₆H₅ and the meaning for the combination of R¹, R², R³, R⁴, R⁹ and R¹⁰ for each individual compound corresponds in each case to one line of Table B.

Table 1d Compounds of formula I.D in which o is 0, Y is Br and the meaning for the combination of R¹, R², R³, R⁴, R⁹ and R¹⁰ for each individual compound corresponds in each case to one line of Table B.

Table 2d Compounds of formula I.D in which o is 1, R⁷⁸ is 2″-F, Y is Br and the meaning for the combination of R¹, R², R³, R⁴, R⁹ and R¹⁰ for each individual compound corresponds in each case to one line of Table B.

Table 3d Compounds of formula I.D in which o is 1, R⁷⁸ is 2″-Cl, Y is Br and the meaning for the combination of R¹, R², R³, R⁴, R⁹ and R¹⁰ for each individual compound corresponds in each case to one line of Table B.

Table 4d Compounds of formula I.D in which o is 1, R⁷⁸ is 2″-Br, Y is Br and the meaning for the combination of R¹, R², R³, R⁴, R⁹ and R¹⁰ for each individual compound corresponds in each case to one line of Table B.

Table 5d Compounds of formula I.D in which o is 1, R⁷⁸ is 2″-CH₃, Y is Br and the meaning for the combination of R¹, R², R³, R⁴, R⁹ and R¹⁰ for each individual compound corresponds in each case to one line of Table B.

Table 6d Compounds of formula I.D in which o is 1, R⁷⁸ is 2″-OCH₃, Y is Br and the meaning for the combination of R¹, R², R³, R⁴, R⁹ and R¹⁰ for each individual compound corresponds in each case to one line of Table B.

Table 7d Compounds of formula I.D in which o is 1, R⁷⁸ is 2″-OCHF₂, Y is Br and the meaning for the combination of R¹, R², R³, R⁴, R⁹ and R¹⁰ for each individual compound corresponds in each case to one line of Table B. Table 8d Compounds of formula I.D in which o is 1, R⁷⁸ is 2″-C₆H₅, Y is Br and the meaning for the combination of R¹, R², R³, R⁴, R⁹ and R¹⁰ for each individual compound corresponds in each case to one line of Table B.

Table 9d Compounds of formula I.D in which o is 0, Y is CH₃ and the meaning for the combination of R¹, R², R³, R⁴, R⁹ and R¹⁰ for each individual compound corresponds in each case to one line of Table B.

Table 10d Compounds of formula I.D in which o is 1, R⁷⁸ is 2″-F, Yis CH₃ and the meaning for the combination of R¹, R², R³, R⁴, R⁹ and R¹⁰ for each individual compound corresponds in each case to one line of Table B.

Table 11d Compounds of formula I.D in which o is 1, R⁷⁸ is 2″-Cl, Yis CH₃ and the meaning for the combination of R¹, R², R³, R⁴, R⁹ and R¹⁰ for each individual compound corresponds in each case to one line of Table B.

Table 12d Compounds of formula I.D in which o is 1, R⁷⁸ is 2″-Br, Yis CH₃ and the meaning for the combination of R¹, R², R³, R⁴, R⁹ and R¹⁰ for each individual compound corresponds in each case to one line of Table B.

Table 13d Compounds of formula I.D in which o is 1, R⁷⁸ is 2″-CH₃, Yis CH₃ and the meaning for the combination of R¹, R², R³, R⁴, R⁹ and R¹⁰ for each individual compound corresponds in each case to one line of Table B.

Table 14d Compounds of formula I.D in which o is 1, R⁷⁸ is 2″-OCH₃, Yis CH₃ and the meaning for the combination of R¹, R², R³, R⁴, R⁹ and R¹⁰ for each individual compound corresponds in each case to one line of Table B.

Table 15d Compounds of formula I.D in which o is 1, R⁷⁸ is 2″-OCHF₂, Yis CH₃ and the meaning for the combination of R¹, R², R³, R⁴, R⁹ and R¹⁰ for each individual compound corresponds in each case to one line of Table B.

Table 16d Compounds of formula I.D in which o is 1, R⁷⁸ is 2″-C₆H₅, Yis CH₃ and the meaning for the combination of R¹, R², R³, R⁴, R⁹ and R¹⁰ for each individual compound corresponds in each case to one line of Table B.

Table 17d Compounds of formula I.D in which o is 0, Yis CF₃ and the meaning for the combination of R¹, R², R³, R⁴, R⁹ and R¹⁰ for each individual compound corresponds in each case to one line of Table B.

Table 18d Compounds of formula I.D in which o is 1, R⁷⁸ is 2″-F, Yis CF₃ and the meaning for the combination of R¹, R², R³, R⁴, R⁹ and R¹⁰ for each individual compound corresponds in each case to one line of Table B.

Table 19d Compounds of formula I.D in which o is 1, R⁷⁸ is 2″-Cl, Yis CF₃ and the meaning for the combination of R¹, R², R³, R⁴, R⁹ and R¹⁰ for each individual compound corresponds in each case to one line of Table B.

Table 20d Compounds of formula I.D in which o is 1, R⁷⁸ is 2″-Br, Yis CF₃ and the meaning for the combination of R¹, R², R³, R⁴, R⁹ and R¹⁰ for each individual compound corresponds in each case to one line of Table B.

Table 21d Compounds of formula I.D in which o is 1, R⁷⁸ is 2″-CH₃, Yis CF₃ and the meaning for the combination of R¹, R², R³, R⁴, R⁹ and R¹⁰ for each individual compound corresponds in each case to one line of Table B.

Table 22d Compounds of formula I.D in which o is 1, R⁷⁸ is 2″-OCH₃, Y is CF₃ and the meaning for the combination of R¹, R², R³, R⁴, R⁹ and R¹⁰ for each individual compound corresponds in each case to one line of Table B.

Table 23d Compounds of formula I.D in which o is 1, R⁷⁸ is 2″-OCHF₂, Y is CF₃ and the meaning for the combination of R¹, R², R³, R⁴, R⁹ and R¹⁰ for each individual compound corresponds in each case to one line of Table B.

Table 24d Compounds of formula I.D in which o is 1, R⁷⁸ is 2″-C₆H₅, Y is CF₃ and the meaning for the combination of R¹, R², R³, R⁴, R⁹ and R¹⁰ for each individual compound corresponds in each case to one line of Table B.

Table 25d Compounds of formula I.D in which o is 0, Y is CH(CH₃)₂ and the meaning for the combination of R¹, R², R³, R⁴, R⁹ and R¹⁰ for each individual compound corresponds in each case to one line of Table B.

Table 26d Compounds of formula I.D in which o is 1, R⁷⁸ is 2″-F, Y is CH(CH₃)₂ and the meaning for the combination of R¹, R², R³, R⁴, R⁹ and R¹⁰ for each individual compound corresponds in each case to one line of Table B.

Table 27d Compounds of formula I.D in which o is 1, R⁷⁸ is 2″-Cl, Y is CH(CH₃)₂ and the meaning for the combination of R¹, R², R³, R⁴, R⁹ and R¹⁰ for each individual compound corresponds in each case to one line of Table B.

Table 28d Compounds of formula I.D in which o is 1, R⁷⁸ is 2″-Br, Yis CH(CH₃)₂ and the meaning for the combination of R¹, R², R³, R⁴, R⁹ and R¹⁰ for each individual compound corresponds in each case to one line of Table B.

Table 29d Compounds of formula I.D in which o is 1, R⁷⁸ is 2″-CH₃, Y is CH(CH₃)₂ and the meaning for the combination of R¹, R², R³, R⁴, R⁹ and R¹⁰ for each individual compound corresponds in each case to one line of Table B.

Table 30d Compounds of formula I.D in which o is 1, R⁷⁸ is 2″-OCH₃, Y is CH(CH₃)₂ and the meaning for the combination of R¹, R², R³, R⁴, R⁹ and R¹⁰ for each individual compound corresponds in each case to one line of Table B.

Table 31d Compounds of formula I.D in which o is 1, R⁷⁸ is 2″-OCHF₂, Yis CH(CH₃)₂ and the meaning for the combination of R¹, R², R³, R⁴, R⁹ and R¹⁰ for each individual compound corresponds in each case to one line of Table B.

Table 32d Compounds of formula I.D in which o is 1, R⁷⁸ is 2″-C₆H₅, Y is CH(CH₃)₂ and the meaning for the combination of R¹, R², R³, R⁴, R⁹ and R¹⁰ for each individual compound corresponds in each case to one line of Table B.

Table 33d Compounds of formula I.D in which o is 0, Y is C₂H₅ and the meaning for the combination of R¹, R², R³, R⁴, R⁹ and R¹⁰ for each individual compound corresponds in each case to one line of Table B.

Table 34d Compounds of formula I.D in which o is 1, R⁷⁸ is 2″-F, Y is C₂H₅ and the meaning for the combination of R¹, R², R³, R⁴, R⁹ and R¹⁰ for each individual compound corresponds in each case to one line of Table B.

Table 35d Compounds of formula I.D in which o is 1, R⁷⁸ is 2″-Cl, Y is C₂H₅ and the meaning for the combination of R¹, R², R³, R⁴, R⁹ and R¹⁰ for each individual compound corresponds in each case to one line of Table B.

Table 36d Compounds of formula I.D in which o is 1, R⁷⁸ is 2″-Br, Yis C₂H₅ and the meaning for the combination of R¹, R², R³, R⁴, R⁹ and R¹⁰ for each individual compound corresponds in each case to one line of Table B.

Table 37d Compounds of formula I.D in which o is 1, R⁷⁸ is 2″-CH₃, Y is C₂H₅ and the meaning for the combination of R¹, R², R³, R⁴, R⁹ and R¹⁰ for each individual compound corresponds in each case to one line of Table B.

Table 38d Compounds of formula I.D in which o is 1, R⁷⁸ is 2″-OCH₃, Y is C₂H₅ and the meaning for the combination of R¹, R², R³, R⁴, R⁹ and R¹⁰ for each individual compound corresponds in each case to one line of Table B.

Table 39d Compounds of formula I.D in which o is 1, R⁷⁸ is 2″-OCHF₂, Y is C₂H₅ and the meaning for the combination of R¹, R², R³, R⁴, R⁹ and R¹⁰ for each individual compound corresponds in each case to one line of Table B.

Table 40d Compounds of formula I.D in which o is 1, R⁷⁸ is 2″-C₆H₅, Y is C₂H₅ and the meaning for the combination of R¹, R², R³, R⁴, R⁹ and R¹⁰ for each individual compound corresponds in each case to one line of Table B.

Table 41d Compounds of formula I.D in which o is 0, Yis CHF₂ and the meaning for the combination of R¹, R², R³, R⁴, R⁹ and R¹⁰ for each individual compound corresponds in each case to one line of Table B.

Table 42d Compounds of formula I.D in which o is 1, R⁷⁸ is 2″-F, Y is CHF₂ and the meaning for the combination of R¹, R², R³, R⁴, R⁹ and R¹⁰ for each individual compound corresponds in each case to one line of Table B.

Table 43d Compounds of formula I.D in which o is 1, R⁷⁸ is 2″-Cl, Yis CHF₂ and the meaning for the combination of R¹, R², R³, R⁴, R⁹ and R¹⁰ for each individual compound corresponds in each case to one line of Table B.

Table 44d Compounds of formula I.D in which o is 1, R⁷⁸ is 2″-Br, Yis CHF₂ and the meaning for the combination of R¹, R², R³, R⁴, R⁹ and R¹⁰ for each individual compound corresponds in each case to one line of Table B.

Table 45d Compounds of formula I.D in which o is 1, R⁷⁸ is 2″-CH₃, Y is CHF₂ and the meaning for the combination of R¹, R², R³, R⁴, R⁹ and R¹⁰ for each individual compound corresponds in each case to one line of Table B.

Table 46d Compounds of formula I.D in which o is 1, R⁷⁸ is 2″-OCH₃, Y is CHF₂ and the meaning for the combination of R¹, R², R³, R⁴, R⁹ and R¹⁰ for each individual compound corresponds in each case to one line of Table B.

Table 47d Compounds of formula I.D in which o is 1, R⁷⁸ is 2″-OCHF₂, Y is CHF₂ and the meaning for the combination of R¹, R², R³, R⁴, R⁹ and R¹⁰ for each individual compound corresponds in each case to one line of Table B.

Table 48d Compounds of formula I.D in which o is 1, R⁷⁸ is 2″-C₆H₅, Y is CHF₂ and the meaning for the combination of R¹, R², R³, R⁴, R⁹ and R¹⁰ for each individual compound corresponds in each case to one line of Table B.

Table 49d Compounds of formula I.D in which o is 0, Y is CN and the meaning for the combination of R¹, R², R³, R⁴, R⁹ and R¹⁰ for each individual compound corresponds in each case to one line of Table B.

Table 50d Compounds of formula I.D in which o is 1, R⁷⁸ is 2″-F, Y is CN and the meaning for the combination of R¹, R², R³, R⁴, R⁹ and R¹⁰ for each individual compound corresponds in each case to one line of Table B.

Table 51d Compounds of formula I.D in which o is 1, R⁷⁸ is 2″-Cl, Yis CN and the meaning for the combination of R¹, R², R³, R⁴, R⁹ and R¹⁰ for each individual compound corresponds in each case to one line of Table B.

Table 52d Compounds of formula I.D in which o is 1, R⁷⁸ is 2″-Br, Yis CN and the meaning for the combination of R¹, R², R³, R⁴, R⁹ and R¹⁰ for each individual compound corresponds in each case to one line of Table B.

Table 53d Compounds of formula I.D in which o is 1, R⁷⁸ is 2″-CH₃, Y is CN and the meaning for the combination of R¹, R², R³, R⁴, R⁹ and R¹⁰ for each individual compound corresponds in each case to one line of Table B.

Table 54d Compounds of formula I.D in which o is 1, R⁷⁸ is 2″-OCH₃, Y is CN and the meaning for the combination of R¹, R², R³, R⁴, R⁹ and R¹⁰ for each individual compound corresponds in each case to one line of Table B.

Table 55d Compounds of formula I.D in which o is 1, R⁷⁸ is 2″-OCHF₂, Y is CN and the meaning for the combination of R¹, R², R³, R⁴, R⁹ and R¹⁰ for each individual compound corresponds in each case to one line of Table B.

Table 56d Compounds of formula I.D in which o is 1, R⁷⁸ is 2″-C₆H₅, Y is CN and the meaning for the combination of R¹, R², R³, R⁴, R⁹ and R¹⁰ for each individual compound corresponds in each case to one line of Table B.

Table 57d Compounds of formula I.D in which o is 0, Yis OCHF₂ and the meaning for the combination of R¹, R², R³, R⁴, R⁹ and R¹⁰ for each individual compound corresponds in each case to one line of Table B.

Table 58d Compounds of formula I.D in which o is 1, R⁷⁸ is 2″-F, Y is OCHF₂ and the meaning for the combination of R¹, R², R³, R⁴, R⁹ and R¹⁰ for each individual compound corresponds in each case to one line of Table B.

Table 59d Compounds of formula I.D in which o is 1, R⁷⁸ is 2″-Cl, Y is OCHF₂ and the meaning for the combination of R¹, R², R³, R⁴, R⁹ and R¹⁰ for each individual compound corresponds in each case to one line of Table B.

Table 60d Compounds of formula I.D in which o is 1, R⁷⁸ is 2″-Br, Yis OCHF₂ and the meaning for the combination of R¹, R², R³, R⁴, R⁹ and R¹⁰ for each individual compound corresponds in each case to one line of Table B.

Table 61d Compounds of formula I.D in which o is 1, R⁷⁸ is 2″-CH₃, Yis OCHF₂ and the meaning for the combination of R¹, R², R³, R⁴, R⁹ and R¹⁰ for each individual compound corresponds in each case to one line of Table B.

Table 62d Compounds of formula I.D in which o is 1, R⁷⁸ is 2″-OCH₃, Y is OCHF₂ and the meaning for the combination of R¹, R², R³, R⁴, R⁹ and R¹⁰ for each individual compound corresponds in each case to one line of Table B.

Table 63d Compounds of formula I.D in which o is 1, R⁷⁸ is 2″-OCHF₂, Y is OCHF₂ and the meaning for the combination of R¹, R², R³, R⁴, R⁹ and R¹⁰ for each individual compound corresponds in each case to one line of Table B.

Table 64d Compounds of formula I.D in which o is 1, R⁷⁸ is 2″-C₆H₅, Y is OCHF₂ and the meaning for the combination of R¹, R², R³, R⁴, R⁹ and R¹⁰ for each individual compound corresponds in each case to one line of Table B.

Table 65d Compounds of formula I.D in which o is 0, Y is Cl and the meaning for the combination of R¹, R², R³, R⁴, R⁹ and R¹⁰ for each individual compound corresponds in each case to one line of Table B.

Table 66d Compounds of formula I.D in which o is 1, R⁷⁸ is 2″-F, Y is Cl and the meaning for the combination of R¹, R², R³, R⁴, R⁹ and R¹⁰ for each individual compound corresponds in each case to one line of Table B.

Table 67d Compounds of formula I.D in which o is 1, R⁷⁸ is 2″-Cl, Y is Cl and the meaning for the combination of R¹, R², R³, R⁴, R⁹ and R¹⁰ for each individual compound corresponds in each case to one line of Table B.

Table 68d Compounds of formula I.D in which o is 1, R⁷⁸ is 2″-Br, Y is Cl and the meaning for the combination of R¹, R², R³, R⁴, R⁹ and R¹⁰ for each individual compound corresponds in each case to one line of Table B.

Table 69d Compounds of formula I.D in which o is 1, R⁷⁸ is 2″-CH₃, Y is Cl and the meaning for the combination of R¹, R², R³, R⁴, R⁹ and R¹⁰ for each individual compound corresponds in each case to one line of Table B.

Table 70d Compounds of formula I.D in which o is 1, R⁷⁸ is 2″-OCH₃, Y is Cl and the meaning for the combination of R¹, R², R³, R⁴, R⁹ and R¹⁰ for each individual compound corresponds in each case to one line of Table B.

Table 71d Compounds of formula I.D in which o is 1, R⁷⁸ is 2″-OCHF₂, Y is Cl and the meaning for the combination of R¹, R², R³, R⁴, R⁹ and R¹⁰ for each individual compound corresponds in each case to one line of Table B.

Table 72d Compounds of formula I.D in which o is 1, R⁷⁸ is 2″-C₆H₅, Y is Cl and the meaning for the combination of R¹, R², R³, R⁴, R⁹ and R¹⁰ for each individual compound corresponds in each case to one line of Table B.

Table 73d Compounds of formula I.D in which o is 0, Y is F and the meaning for the combination of R¹, R², R³, R⁴, R⁹ and R¹⁰ for each individual compound corresponds in each case to one line of Table B.

Table 74d Compounds of formula I.D in which o is 1, R⁷⁸ is 2″-F, Y is F and the meaning for the combination of R¹, R², R³, R⁴, R⁹ and R¹⁰ for each individual compound corresponds in each case to one line of Table B.

Table 75d Compounds of formula I.D in which o is 1, R⁷⁸ is 2″-Cl, Y is F and the meaning for the combination of R¹, R², R³, R⁴, R⁹ and R¹⁰ for each individual compound corresponds in each case to one line of Table B.

Table 76d Compounds of formula I.D in which o is 1, R⁷⁸ is 2″-Br, Y is F and the meaning for the combination of R¹, R², R³, R⁴, R⁹ and R¹⁰ for each individual compound corresponds in each case to one line of Table B.

Table 77d Compounds of formula I.D in which o is 1, R⁷⁸ is 2″-CH₃, Y is F and the meaning for the combination of R¹, R², R³, R⁴, R⁹ and R¹⁰ for each individual compound corresponds in each case to one line of Table B.

Table 78d Compounds of formula I.D in which o is 1, R⁷⁸ is 2″-OCH₃, Y is F and the meaning for the combination of R¹, R², R³, R⁴, R⁹ and R¹⁰ for each individual compound corresponds in each case to one line of Table B.

Table 79d Compounds of formula I.D in which o is 1, R⁷⁸ is 2″-OCHF₂, Y is F and the meaning for the combination of R¹, R², R³, R⁴, R⁹ and R¹⁰ for each individual compound corresponds in each case to one line of Table B.

Table 80d Compounds of formula I.D in which o is 1, R⁷⁸ is 2″-C₆H₅, Y is F and the meaning for the combination of R¹, R², R³, R⁴, R⁹ and R¹⁰ for each individual compound corresponds in each case to one line of Table B.

Table 1e Compounds of formula I.E in which o is 0, Y is Br and the meaning for the combination of R¹, R², R³, R⁴, R⁹ and R¹⁰ for each individual compound corresponds in each case to one line of Table B.

Table 2e Compounds of formula I.E in which o is 1, R⁷⁸ is 1″-F, Y is Br and the meaning for the combination of R¹, R², R³, R⁴, R⁹ and R¹⁰ for each individual compound corresponds in each case to one line of Table B.

Table 3e Compounds of formula I.E in which o is 1, R⁷⁸ is 1″-Cl, Y is Br and the meaning for the combination of R¹, R², R³, R⁴, R⁹ and R¹⁰ for each individual compound corresponds in each case to one line of Table B.

Table 4e Compounds of formula I.E in which o is 1, R⁷⁸ is 1″-Br, Y is Br and the meaning for the combination of R¹, R², R³, R⁴, R⁹ and R¹⁰ for each individual compound corresponds in each case to one line of Table B.

Table 5e Compounds of formula I.E in which o is 1, R⁷⁸ is 1″-CH₃, Y is Br and the meaning for the combination of R¹, R², R³, R⁴, R⁹ and R¹⁰ for each individual compound corresponds in each case to one line of Table B.

Table 6e Compounds of formula I.E in which o is 1, R⁷⁸ is 1″-OCH₃, Y is Br and the meaning for the combination of R¹, R², R³, R⁴, R⁹ and R¹⁰ for each individual compound corresponds in each case to one line of Table B.

Table 7e Compounds of formula I.E in which o is 1, R⁷⁸ is 1″-OCHF₂, Y is Br and the meaning for the combination of R¹, R², R³, R⁴, R⁹ and R¹⁰ for each individual compound corresponds in each case to one line of Table B.

Table 8e Compounds of formula I.E in which o is 1, R⁷⁸ is 1″-C₆H₅, Y is Br and the meaning for the combination of R¹, R², R³, R⁴, R⁹ and R¹⁰ for each individual compound corresponds in each case to one line of Table B.

Table 9e Compounds of formula I.E in which o is 0, Y is CH₃ and the meaning for the combination of R¹, R², R³, R⁴, R⁹ and R¹⁰ for each individual compound corresponds in each case to one line of Table B.

Table 10e Compounds of formula I.E in which o is 1, R⁷⁸ is 1″-F, Y is CH₃ and the meaning for the combination of R¹, R², R³, R⁴, R⁹ and R¹⁰ for each individual compound corresponds in each case to one line of Table B.

Table 11e Compounds of formula I.E in which o is 1, R⁷⁸ is 1″-Cl, Y is CH₃ and the meaning for the combination of R¹, R², R³, R⁴, R⁹ and R¹⁰ for each individual compound corresponds in each case to one line of Table B.

Table 12e Compounds of formula I.E in which o is 1, R⁷⁸ is 1″-Br, Y is CH₃ and the meaning for the combination of R¹, R², R³, R⁴, R⁹ and R¹⁰ for each individual compound corresponds in each case to one line of Table B.

Table 13e Compounds of formula I.E in which o is 1, R⁷⁸ is 1″-CH₃, Y is CH₃ and the meaning for the combination of R¹, R², R³, R⁴, R⁹ and R¹⁰ for each individual compound corresponds in each case to one line of Table B.

Table 14e Compounds of formula I.E in which o is 1, R⁷⁸ is 1″-OCH₃, Y is CH₃ and the meaning for the combination of R¹, R², R³, R⁴, R⁹ and R¹⁰ for each individual compound corresponds in each case to one line of Table B.

Table 15e Compounds of formula I.E in which o is 1, R⁷⁸ is 1″-OCHF₂, Y is CH₃ and the meaning for the combination of R¹, R², R³, R⁴, R⁹ and R¹⁰ for each individual compound corresponds in each case to one line of Table B.

Table 16e Compounds of formula I.E in which o is 1, R⁷⁸ is 1″-C₆H₅, Y is CH₃ and the meaning for the combination of R¹, R², R³, R⁴, R⁹ and R¹⁰ for each individual compound corresponds in each case to one line of Table B.

Table 17e Compounds of formula I.E in which o is 0, Y is CF₃ and the meaning for the combination of R¹, R², R³, R⁴, R⁹ and R¹⁰ for each individual compound corresponds in each case to one line of Table B.

Table 18e Compounds of formula I.E in which o is 1, R⁷⁸ is 1″-F, Y is CF₃ and the meaning for the combination of R¹, R², R³, R⁴, R⁹ and R¹⁰ for each individual compound corresponds in each case to one line of Table B.

Table 19e Compounds of formula I.E in which o is 1, R⁷⁸ is 1″-Cl, Y is CF₃ and the meaning for the combination of R¹, R², R³, R⁴, R⁹ and R¹⁰ for each individual compound corresponds in each case to one line of Table B.

Table 20e Compounds of formula I.E in which o is 1, R⁷⁸ is 1″-Br, Y is CF₃ and the meaning for the combination of R¹, R², R³, R⁴, R⁹ and R¹⁰ for each individual compound corresponds in each case to one line of Table B.

Table 21e Compounds of formula I.E in which o is 1, R⁷⁸ is 1″-CH₃, Y is CF₃ and the meaning for the combination of R¹, R², R³, R⁴, R⁹ and R¹⁰ for each individual compound corresponds in each case to one line of Table B.

Table 22e Compounds of formula I.E in which o is 1, R⁷⁸ is 1″-OCH₃, Y is CF₃ and the meaning for the combination of R¹, R², R³, R⁴, R⁹ and R¹⁰ for each individual compound corresponds in each case to one line of Table B.

Table 23e Compounds of formula I.E in which o is 1, R⁷⁸ is 1″-OCHF₂, Y is CF₃ and the meaning for the combination of R¹, R², R³, R⁴, R⁹ and R¹⁰ for each individual compound corresponds in each case to one line of Table B.

Table 24e Compounds of formula I.E in which o is 1, R⁷⁸ is 1″-C₆H₅, Y is CF₃ and the meaning for the combination of R¹, R², R³, R⁴, R⁹ and R¹⁰ for each individual compound corresponds in each case to one line of Table B.

Table 25e Compounds of formula I.E in which o is 0, Y is CH(CH₃)₂ and the meaning for the combination of R¹, R², R³, R⁴, R⁹ and R¹⁰ for each individual compound corresponds in each case to one line of Table B.

Table 26e Compounds of formula I.E in which o is 1, R⁷⁸ is 1″-F, Y is CH(CH₃)₂ and the meaning for the combination of R¹, R², R³, R⁴, R⁹ and R¹⁰ for each individual compound corresponds in each case to one line of Table B.

Table 27e Compounds of formula I.E in which o is 1, R⁷⁸ is 1″-Cl, Y is CH(CH₃)₂ and the meaning for the combination of R¹, R², R³, R⁴, R⁹ and R¹⁰ for each individual compound corresponds in each case to one line of Table B.

Table 28e Compounds of formula I.E in which o is 1, R⁷⁸ is 1″-Br, Y is CH(CH₃)₂ and the meaning for the combination of R¹, R², R³, R⁴, R⁹ and R¹⁰ for each individual compound corresponds in each case to one line of Table B.

Table 29e Compounds of formula I.E in which o is 1, R⁷⁸ is 1″-CH₃, Y is CH(CH₃)₂ and the meaning for the combination of R¹, R², R³, R⁴, R⁹ and R¹⁰ for each individual compound corresponds in each case to one line of Table B.

Table 30e Compounds of formula I.E in which o is 1, R⁷⁸ is 1″-OCH₃, Y is CH(CH₃)₂ and the meaning for the combination of R¹, R², R³, R⁴, R⁹ and R¹⁰ for each individual compound corresponds in each case to one line of Table B.

Table 31e Compounds of formula I.E in which o is 1, R⁷⁸ is 1″-OCHF₂, Y is CH(CH₃)₂ and the meaning for the combination of R¹, R², R³, R⁴, R⁹ and R¹⁰ for each individual compound corresponds in each case to one line of Table B.

Table 32e Compounds of formula I.E in which o is 1, R⁷⁸ is 1″-C₆H₅, Y is CH(CH₃)₂ and the meaning for the combination of R¹, R², R³, R⁴, R⁹ and R¹⁰ for each individual compound corresponds in each case to one line of Table B.

Table 33e Compounds of formula I.E in which o is 0, Y is C₂H₅ and the meaning for the combination of R¹, R², R³, R⁴, R⁹ and R¹⁰ for each individual compound corresponds in each case to one line of Table B.

Table 34e Compounds of formula I.E in which o is 1, R⁷⁸ is 1″-F, Y is C₂H₅ and the meaning for the combination of R¹, R², R³, R⁴, R⁹ and R¹⁰ for each individual compound corresponds in each case to one line of Table B.

Table 35e Compounds of formula I.E in which o is 1, R⁷⁸ is 1″-Cl, Y is C₂H₅ and the meaning for the combination of R¹, R², R³, R⁴, R⁹ and R¹⁰ for each individual compound corresponds in each case to one line of Table B.

Table 36e Compounds of formula I.E in which o is 1, R⁷⁸ is 1″-Br, Y is C₂H₅ and the meaning for the combination of R¹, R², R³, R⁴, R⁹ and R¹⁰ for each individual compound corresponds in each case to one line of Table B.

Table 37e Compounds of formula I.E in which o is 1, R⁷⁸ is 1″-CH₃, Y is C₂H₅ and the meaning for the combination of R¹, R², R³, R⁴, R⁹ and R¹⁰ for each individual compound corresponds in each case to one line of Table B.

Table 38e Compounds of formula I.E in which o is 1, R⁷⁸ is 1″-OCH₃, Y is C₂H₅ and the meaning for the combination of R¹, R², R³, R⁴, R⁹ and R¹⁰ for each individual compound corresponds in each case to one line of Table B.

Table 39e Compounds of formula I.E in which o is 1, R⁷⁸ is 1″-OCHF₂, Y is C₂H₅ and the meaning for the combination of R¹, R², R³, R⁴, R⁹ and R¹⁰ for each individual compound corresponds in each case to one line of Table B.

Table 40e Compounds of formula I.E in which o is 1, R⁷⁸ is 1″-C₆H₅, Y is C₂H₅ and the meaning for the combination of R¹, R², R³, R⁴, R⁹ and R¹⁰ for each individual compound corresponds in each case to one line of Table B.

Table 41e Compounds of formula I.E in which o is 0, Y is CHF₂ and the meaning for the combination of R¹, R², R³, R⁴, R⁹ and R¹⁰ for each individual compound corresponds in each case to one line of Table B.

Table 42e Compounds of formula I.E in which o is 1, R⁷⁸ is 1″-F, Y is CHF₂ and the meaning for the combination of R¹, R², R³, R⁴, R⁹ and R¹⁰ for each individual compound corresponds in each case to one line of Table B.

Table 43e Compounds of formula I.E in which o is 1, R⁷⁸ is 1″-Cl, Y is CHF₂ and the meaning for the combination of R¹, R², R³, R⁴, R⁹ and R¹⁰ for each individual compound corresponds in each case to one line of Table B.

Table 44e Compounds of formula I.E in which o is 1, R⁷⁸ is 1″-Br, Y is CHF₂ and the meaning for the combination of R¹, R², R³, R⁴, R⁹ and R¹⁰ for each individual compound corresponds in each case to one line of Table B.

Table 45e Compounds of formula I.E in which o is 1, R⁷⁸ is 1″-CH₃, Y is CHF₂ and the meaning for the combination of R¹, R², R³, R⁴, R⁹ and R¹⁰ for each individual compound corresponds in each case to one line of Table B.

Table 46e Compounds of formula I.E in which o is 1, R⁷⁸ is 1″-OCH₃, Y is CHF₂ and the meaning for the combination of R¹, R², R³, R⁴, R⁹ and R¹⁰ for each individual compound corresponds in each case to one line of Table B.

Table 47e Compounds of formula I.E in which o is 1, R⁷⁸ is 1″-OCHF₂, Yis CHF₂ and the meaning for the combination of R¹, R², R³, R⁴, R⁹ and R¹⁰ for each individual compound corresponds in each case to one line of Table B.

Table 48e Compounds of formula I.E in which o is 1, R⁷⁸ is 1″-C₆H₅, Yis CHF₂ and the meaning for the combination of R¹, R², R³, R⁴, R⁹ and R¹⁰ for each individual compound corresponds in each case to one line of Table B.

Table 49e Compounds of formula I.E in which o is 0, Y is CN and the meaning for the combination of R¹, R², R³, R⁴, R⁹ and R¹⁰ for each individual compound corresponds in each case to one line of Table B.

Table 50e Compounds of formula I.E in which o is 1, R⁷⁸ is 1″-F, Yis CN and the meaning for the combination of R¹, R², R³, R⁴, R⁹ and R¹⁰ for each individual compound corresponds in each case to one line of Table B.

Table 51e Compounds of formula I.E in which o is 1, R⁷⁸ is 1″-Cl, Yis CN and the meaning for the combination of R¹, R², R³, R⁴, R⁹ and R¹⁰ for each individual compound corresponds in each case to one line of Table B.

Table 52e Compounds of formula I.E in which o is 1, R⁷⁸ is 1″-Br, Yis CN and the meaning for the combination of R¹, R², R³, R⁴, R⁹ and R¹⁰ for each individual compound corresponds in each case to one line of Table B.

Table 53e Compounds of formula I.E in which o is 1, R⁷⁸ is 1″-CH₃, Yis CN and the meaning for the combination of R¹, R², R³, R⁴, R⁹ and R¹⁰ for each individual compound corresponds in each case to one line of Table B.

Table 54e Compounds of formula I.E in which o is 1, R⁷⁸ is 1″-OCH₃, Yis CN and the meaning for the combination of R¹, R², R³, R⁴, R⁹ and R¹⁰ for each individual compound corresponds in each case to one line of Table B.

Table 55e Compounds of formula I.E in which o is 1, R⁷⁸ is 1″-OCHF₂, Yis CN and the meaning for the combination of R¹, R², R³, R⁴, R⁹ and R¹⁰ for each individual compound corresponds in each case to one line of Table B.

Table 56e Compounds of formula I.E in which o is 1, R⁷⁸ is 1″-C₆H₅, Yis CN and the meaning for the combination of R¹, R², R³, R⁴, R⁹ and R¹⁰ for each individual compound corresponds in each case to one line of Table B.

Table 57e Compounds of formula I.E in which o is 0, Y is OCHF₂ and the meaning for the combination of R¹, R², R³, R⁴, R⁹ and R¹⁰ for each individual compound corresponds in each case to one line of Table B.

Table 58e Compounds of formula I.E in which o is 1, R⁷⁸ is 1″-F, Yis OCHF₂ and the meaning for the combination of R¹, R², R³, R⁴, R⁹ and R¹⁰ for each individual compound corresponds in each case to one line of Table B.

Table 59e Compounds of formula I.E in which o is 1, R⁷⁸ is 1″-Cl, Yis OCHF₂ and the meaning for the combination of R¹, R², R³, R⁴, R⁹ and R¹⁰ for each individual compound corresponds in each case to one line of Table B.

Table 60e Compounds of formula I.E in which o is 1, R⁷⁸ is 1″-Br, Yis OCHF₂ and the meaning for the combination of R¹, R², R³, R⁴, R⁹ and R¹⁰ for each individual compound corresponds in each case to one line of Table B.

Table 61e Compounds of formula I.E in which o is 1, R⁷⁸ is 1″-CH₃, Yis OCHF₂ and the meaning for the combination of R¹, R², R³, R⁴, R⁹ and R¹⁰ for each individual compound corresponds in each case to one line of Table B.

Table 62e Compounds of formula I.E in which o is 1, R⁷⁸ is 1″-OCH₃, Yis OCHF₂ and the meaning for the combination of R¹, R², R³, R⁴, R⁹ and R¹⁰ for each individual compound corresponds in each case to one line of Table B.

Table 63e Compounds of formula I.E in which o is 1, R⁷⁸ is 1″-OCHF₂, Yis OCHF₂ and the meaning for the combination of R¹, R², R³, R⁴, R⁹ and R¹⁰ for each individual compound corresponds in each case to one line of Table B.

Table 64e Compounds of formula I.E in which o is 1, R⁷⁸ is 1″-C₆H₅, Yis OCHF₂ and the meaning for the combination of R¹, R², R³, R⁴, R⁹ and R¹⁰ for each individual compound corresponds in each case to one line of Table B.

Table 65e Compounds of formula I.E in which o is 0, Y is Cl and the meaning for the combination of R¹, R², R³, R⁴, R⁹ and R¹⁰ for each individual compound corresponds in each case to one line of Table B.

Table 66e Compounds of formula I.E in which o is 1, R⁷⁸ is 1″-F, Y is Cl and the meaning for the combination of R¹, R², R³, R⁴, R⁹ and R¹⁰ for each individual compound corresponds in each case to one line of Table B.

Table 67e Compounds of formula I.E in which o is 1, R⁷⁸ is 1″-Cl, Y is Cl and the meaning for the combination of R¹, R², R³, R⁴, R⁹ and R¹⁰ for each individual compound corresponds in each case to one line of Table B.

Table 68e Compounds of formula I.E in which o is 1, R⁷⁸ is 1″-Br, Y is Cl and the meaning for the combination of R¹, R², R³, R⁴, R⁹ and R¹⁰ for each individual compound corresponds in each case to one line of Table B.

Table 69e Compounds of formula I.E in which o is 1, R⁷⁸ is 1″-CH₃, Y is Cl and the meaning for the combination of R¹, R², R³, R⁴, R⁹ and R¹⁰ for each individual compound corresponds in each case to one line of Table B.

Table 70e Compounds of formula I.E in which o is 1, R⁷⁸ is 1″-OCH₃, Y is Cl and the meaning for the combination of R¹, R², R³, R⁴, R⁹ and R¹⁰ for each individual compound corresponds in each case to one line of Table B.

Table 71e Compounds of formula I.E in which o is 1, R⁷⁸ is 1″-OCHF₂, Y is Cl and the meaning for the combination of R¹, R², R³, R⁴, R⁹ and R¹⁰ for each individual compound corresponds in each case to one line of Table B.

Table 72e Compounds of formula I.E in which o is 1, R⁷⁸ is 1″-C₆H₅, Y is Cl and the meaning for the combination of R¹, R², R³, R⁴, R⁹ and R¹⁰ for each individual compound corresponds in each case to one line of Table B.

Table 73e Compounds of formula I.E in which o is 0, Y is F and the meaning for the combination of R¹, R², R³, R⁴, R⁹ and R¹⁰ for each individual compound corresponds in each case to one line of Table B.

Table 74e Compounds of formula I.E in which o is 1, R⁷⁸ is 1″-F, Y is F and the meaning for the combination of R¹, R², R³, R⁴, R⁹ and R¹⁰ for each individual compound corresponds in each case to one line of Table B.

Table 75e Compounds of formula I.E in which o is 1, R⁷⁸ is 1″-Cl, Y is F and the meaning for the combination of R¹, R², R³, R⁴, R⁹ and R¹⁰ for each individual compound corresponds in each case to one line of Table B.

Table 76e Compounds of formula I.E in which o is 1, R⁷⁸ is 1″-Br, Y is F and the meaning for the combination of R¹, R², R³, R⁴, R⁹ and R¹⁰ for each individual compound corresponds in each case to one line of Table B.

Table 77e Compounds of formula I.E in which o is 1, R⁷⁸ is 1″-CH₃, Y is F and the meaning for the combination of R¹, R², R³, R⁴, R⁹ and R¹⁰ for each individual compound corresponds in each case to one line of Table B.

Table 78e Compounds of formula I.E in which o is 1, R⁷⁸ is 1″-OCH₃, Y is F and the meaning for the combination of R¹, R², R³, R⁴, R⁹ and R¹⁰ for each individual compound corresponds in each case to one line of Table B.

Table 79e Compounds of formula I.E in which o is 1, R⁷⁸ is 1″-OCHF₂, Y is F and the meaning for the combination of R¹, R², R³, R⁴, R⁹ and R¹⁰ for each individual compound corresponds in each case to one line of Table B.

Table 80e Compounds of formula I.E in which o is 1, R⁷⁸ is 1″-C₆H₅, Y is F and the meaning for the combination of R¹, R², R³, R⁴, R⁹ and R¹⁰ for each individual compound corresponds in each case to one line of Table B.

Table 1f Compounds of formula I.F in which o is 0 and the meaning for the combination of R¹, R², R³, R⁴, R⁹ and R¹⁰ for each individual compound corresponds in each case to one line of Table B.

Table 2f Compounds of formula I.F in which o is 1, R⁷⁸ is 2″-F and the meaning for the combination of R¹, R², R³, R⁴, R⁹ and R¹⁰ for each individual compound corresponds in each case to one line of Table B.

Table 3f Compounds of formula I.F in which o is 1, R⁷⁸ is 2″-Cl and the meaning for the combination of R¹, R², R³, R⁴, R⁹ and R¹⁰ for each individual compound corresponds in each case to one line of Table B.

Table 4f Compounds of formula I.F in which o is 1, R⁷⁸ is 2″-Br and the meaning for the combination of R¹, R², R³, R⁴, R⁹ and R¹⁰ for each individual compound corresponds in each case to one line of Table B.

Table 5f Compounds of formula I.F in which o is 1, R⁷⁸ is 2″-CH₃ and the meaning for the combination of R¹, R², R³, R⁴, R⁹ and R¹⁰ for each individual compound corresponds in each case to one line of Table B.

Table 6f Compounds of formula I.F in which o is 1, R⁷⁸ is 2″-OCH₃ and the meaning for the combination of R¹, R², R³, R⁴, R⁹ and R¹⁰ for each individual compound corresponds in each case to one line of Table B.

Table 7f Compounds of formula I.F in which o is 1, R⁷⁸ is 2″-OCH F₂ and the meaning for the combination of R¹, R², R³, R⁴, R⁹ and R¹⁰ for each individual compound corresponds in each case to one line of Table B.

Table 8f Compounds of formula I.F in which o is 1, R⁷⁸ is 2″-C₆H₅ and the meaning for the combination of R¹, R², R³, R⁴, R⁹ and R¹⁰ for each individual compound corresponds in each case to one line of Table B.

Table 9f Compounds of formula I.F in which o is 1, R⁷⁸ is 3″-F and the meaning for the combination of R¹, R², R³, R⁴, R⁹ and R¹⁰ for each individual compound corresponds in each case to one line of Table B.

Table 10f Compounds of formula I.F in which o is 1, R⁷⁸ is 3″-Cl and the meaning for the combination of R¹, R², R³, R⁴, R⁹ and R¹⁰ for each individual compound corresponds in each case to one line of Table B.

Table 11f Compounds of formula I.F in which o is 1, R⁷⁸ is 3″-Br and the meaning for the combination of R¹, R², R³, R⁴, R⁹ and R¹⁰ for each individual compound corresponds in each case to one line of Table B.

Table 12f Compounds of formula I.F in which o is 1, R⁷⁸ is 3″-CH₃ and the meaning for the combination of R¹, R², R³, R⁴, R⁹ and R¹⁰ for each individual compound corresponds in each case to one line of Table B.

Table 13f Compounds of formula I.F in which o is 1, R⁷⁸ is 3″-OCH₃ and the meaning for the combination of R¹, R², R³, R⁴, R⁹ and R¹⁰ for each individual compound corresponds in each case to one line of Table B.

Table 14f Compounds of formula I.F in which o is 1, R⁷⁸ is 3″-OCH F₂ and the meaning for the combination of R¹, R², R³, R⁴, R⁹ and R¹⁰ for each individual compound corresponds in each case to one line of Table B.

Table 15f Compounds of formula I.F in which o is 1, R⁷⁸ is 3″-C₆H₅ and the meaning for the combination of R¹, R², R³, R⁴, R⁹ and R¹⁰ for each individual compound corresponds in each case to one line of Table B.

Table 1g Compounds of formula I.G in which o is 0, Yis Br and the meaning for the combination of R¹, R², R³, R⁴, R⁹ and R¹⁰ for each individual compound corresponds in each case to one line of Table B.

Table 2g Compounds of formula I.G in which o is 1, R⁷⁸ is 2″-F, Y is Br and the meaning for the combination of R¹, R², R³, R⁴, R⁹ and R¹⁰ for each individual compound corresponds in each case to one line of Table B.

Table 3g Compounds of formula I.G in which o is 1, R⁷⁸ is 2″-Cl, Y is Br and the meaning for the combination of R¹, R², R³, R⁴, R⁹ and R¹⁰ for each individual compound corresponds in each case to one line of Table B.

Table 4g Compounds of formula I.G in which o is 1, R⁷⁸ is 2″-Br, Y is Br and the meaning for the combination of R¹, R², R³, R⁴, R⁹ and R¹⁰ for each individual compound corresponds in each case to one line of Table B.

Table 5g Compounds of formula I.G in which o is 1, R⁷⁸ is 2″-CH₃, Yis Br and the meaning for the combination of R¹, R², R³, R⁴, R⁹ and R¹⁰ for each individual compound corresponds in each case to one line of Table B.

Table 6g Compounds of formula I.G in which o is 1, R⁷⁸ is 2″-OCH₃, Y is Br and the meaning for the combination of R¹, R², R³, R⁴, R⁹ and R¹⁰ for each individual compound corresponds in each case to one line of Table B.

Table 7g Compounds of formula I.G in which o is 1, R⁷⁸ is 2″-C₆H₅, Y is Br and the meaning for the combination of R¹, R², R³, R⁴, R⁹ and R¹⁰ for each individual compound corresponds in each case to one line of Table B.

Table 8g Compounds of formula I.G in which o is 1, R⁷⁸ is 1″-CH₃, Yis Br and the meaning for the combination of R¹, R², R³, R⁴, R⁹ and R¹⁰ for each individual compound corresponds in each case to one line of Table B.

Table 9g Compounds of formula I.G in which o is 1, R⁷⁸ is 1″-C₆H₅, Y is Br and the meaning for the combination of R¹, R², R³, R⁴, R⁹ and R¹⁰ for each individual compound corresponds in each case to one line of Table B.

Table 10g Compounds of formula I.G in which o is 0, Y is CH₃ and the meaning for the combination of R¹, R², R³, R⁴, R⁹ and R¹⁰ for each individual compound corresponds in each case to one line of Table B.

Table 11g Compounds of formula I.G in which o is 1, R⁷⁸ is 2″-F, Y is CH₃ and the meaning for the combination of R¹, R², R³, R⁴, R⁹ and R¹⁰ for each individual compound corresponds in each case to one line of Table B.

Table 12g Compounds of formula I.G in which o is 1, R⁷⁸ is 2″-Cl, Y is CH₃ and the meaning for the combination of R¹, R², R³, R⁴, R⁹ and R¹⁰ for each individual compound corresponds in each case to one line of Table B.

Table 13g Compounds of formula I.G in which o is 1, R⁷⁸ is 2″-Br, Y is CH₃ and the meaning for the combination of R¹, R², R³, R⁴, R⁹ and R¹⁰ for each individual compound corresponds in each case to one line of Table B.

Table 14g Compounds of formula I.G in which o is 1, R⁷⁸ is 2″-CH₃, Y is CH₃ and the meaning for the combination of R¹, R², R³, R⁴, R⁹ and R¹⁰ for each individual compound corresponds in each case to one line of Table B.

Table 15g Compounds of formula I.G in which o is 1, R⁷⁸ is 2″-OCH₃, Y is CH₃ and the meaning for the combination of R¹, R², R³, R⁴, R⁹ and R¹⁰ for each individual compound corresponds in each case to one line of Table B.

Table 16g Compounds of formula I.G in which o is 1, R⁷⁸ is 2″-C₆H₅, Y is CH₃ and the meaning for the combination of R¹, R², R³, R⁴, R⁹ and R¹⁰ for each individual compound corresponds in each case to one line of Table B.

Table 17g Compounds of formula I.G in which o is 1, R⁷⁸ is 1″-CH₃, Y is CH₃ and the meaning for the combination of R¹, R², R³, R⁴, R⁹ and R¹⁰ for each individual compound corresponds in each case to one line of Table B.

Table 18g Compounds of formula I.G in which o is 1, R⁷⁸ is 1″-C₆H₅, Y is CH₃ and the meaning for the combination of R¹, R², R³, R⁴, R⁹ and R¹⁰ for each individual compound corresponds in each case to one line of Table B.

Table 19g Compounds of formula I.G in which o is 0, Y is CF₃ and the meaning for the combination of R¹, R², R³, R⁴, R⁹ and R¹⁰ for each individual compound corresponds in each case to one line of Table B.

Table 20g Compounds of formula I.G in which o is 1, R⁷⁸ is 2″-F, Y is CF₃ and the meaning for the combination of R¹, R², R³, R⁴, R⁹ and R¹⁰ for each individual compound corresponds in each case to one line of Table B.

Table 21g Compounds of formula I.G in which o is 1, R⁷⁸ is 2″-Cl, Y is CF₃ and the meaning for the combination of R¹, R², R³, R⁴, R⁹ and R¹⁰ for each individual compound corresponds in each case to one line of Table B.

Table 22g Compounds of formula I.G in which o is 1, R⁷⁸ is 2″-Br, Y is CF₃ and the meaning for the combination of R¹, R², R³, R⁴, R⁹ and R¹⁰ for each individual compound corresponds in each case to one line of Table B.

Table 23g Compounds of formula I.G in which o is 1, R⁷⁸ is 2″-CH₃, Y is CF₃ and the meaning for the combination of R¹, R², R³, R⁴, R⁹ and R¹⁰ for each individual compound corresponds in each case to one line of Table B.

Table 24g Compounds of formula I.G in which o is 1, R⁷⁸ is 2″-OCH₃, Y is CF₃ and the meaning for the combination of R¹, R², R³, R⁴, R⁹ and R¹⁰ for each individual compound corresponds in each case to one line of Table B.

Table 25g Compounds of formula I.G in which o is 1, R⁷⁸ is 2″-C₆H₅, Y is CF₃ and the meaning for the combination of R¹, R², R³, R⁴, R⁹ and R¹⁰ for each individual compound corresponds in each case to one line of Table B.

Table 26g Compounds of formula I.G in which o is 1, R⁷⁸ is 1″-CH₃, Y is CF₃ and the meaning for the combination of R¹, R², R³, R⁴, R⁹ and R¹⁰ for each individual compound corresponds in each case to one line of Table B.

Table 27g Compounds of formula I.G in which o is 1, R⁷⁸ is 1″-C₆H₅, Y is CF₃ and the meaning for the combination of R¹, R², R³, R⁴, R⁹ and R¹⁰ for each individual compound corresponds in each case to one line of Table B.

Table 28g Compounds of formula I.G in which o is 0, Y is CH(CH₃)₂ and the meaning for the combination of R¹, R², R³, R⁴, R⁹ and R¹⁰ for each individual compound corresponds in each case to one line of Table B.

Table 29g Compounds of formula I.G in which o is 1, R⁷⁸ is 2″-F, Y is CH(CH₃)₂ and the meaning for the combination of R¹, R², R³, R⁴, R⁹ and R¹⁰ for each individual compound corresponds in each case to one line of Table B.

Table 30g Compounds of formula I.G in which o is 1, R⁷⁸ is 2″-Cl, Y is CH(CH₃)₂ and the meaning for the combination of R¹, R², R³, R⁴, R⁹ and R¹⁰ for each individual compound corresponds in each case to one line of Table B.

Table 31g Compounds of formula I.G in which o is 1, R⁷⁸ is 2″-Br, Y is CH(CH₃)₂ and the meaning for the combination of R¹, R², R³, R⁴, R⁹ and R¹⁰ for each individual compound corresponds in each case to one line of Table B.

Table 32g Compounds of formula I.G in which o is 1, R⁷⁸ is 2″-CH₃, Y is CH(CH₃)₂ and the meaning for the combination of R¹, R², R³, R⁴, R⁹ and R¹⁰ for each individual compound corresponds in each case to one line of Table B.

Table 33g Compounds of formula I.G in which o is 1, R⁷⁸ is 2″-OCH₃, Y is CH(CH₃)₂ and the meaning for the combination of R¹, R², R³, R⁴, R⁹ and R¹⁰ for each individual compound corresponds in each case to one line of Table B.

Table 34g Compounds of formula I.G in which o is 1, R⁷⁸ is 2″-C₆H₅, Y is CH(CH₃)₂ and the meaning for the combination of R¹, R², R³, R⁴, R⁹ and R¹⁰ for each individual compound corresponds in each case to one line of Table B.

Table 35g Compounds of formula I.G in which o is 1, R⁷⁸ is 1″-CH₃, Y is CH(CH₃)₂ and the meaning for the combination of R¹, R², R³, R⁴, R⁹ and R¹⁰ for each individual compound corresponds in each case to one line of Table B.

Table 36g Compounds of formula I.G in which o is 1, R⁷⁸ is 1″-C₆H₅, Y is CH(CH₃)₂ and the meaning for the combination of R¹, R², R³, R⁴, R⁹ and R¹⁰ for each individual compound corresponds in each case to one line of Table B.

Table 37g Compounds of formula I.G in which o is 0, Y is C₂H₅ and the meaning for the combination of R¹, R², R³, R⁴, R⁹ and R¹⁰ for each individual compound corresponds in each case to one line of Table B.

Table 38g Compounds of formula I.G in which o is 1, R⁷⁸ is 2″-F, Y is C₂H₅ and the meaning for the combination of R¹, R², R³, R⁴, R⁹ and R¹⁰ for each individual compound corresponds in each case to one line of Table B.

Table 39g Compounds of formula I.G in which o is 1, R⁷⁸ is 2″-Cl, Y is C₂H₅ and the meaning for the combination of R¹, R², R³, R⁴, R⁹ and R¹⁰ for each individual compound corresponds in each case to one line of Table B.

Table 40g Compounds of formula I.G in which o is 1, R⁷⁸ is 2″-Br, Y is C₂H₅ and the meaning for the combination of R¹, R², R³, R⁴, R⁹ and R¹⁰ for each individual compound corresponds in each case to one line of Table B.

Table 41g Compounds of formula I.G in which o is 1, R⁷⁸ is 2″-CH₃, Y is C₂H₅ and the meaning for the combination of R¹, R², R³, R⁴, R⁹ and R¹⁰ for each individual compound corresponds in each case to one line of Table B.

Table 42g Compounds of formula I.G in which o is 1, R⁷⁸ is 2″-OCH₃, Y is C₂H₅ and the meaning for the combination of R¹, R², R³, R⁴, R⁹ and R¹⁰ for each individual compound corresponds in each case to one line of Table B.

Table 43g Compounds of formula I.G in which o is 1, R⁷⁸ is 2″-C₆H₅, Y is C₂H₅ and the meaning for the combination of R¹, R², R³, R⁴, R⁹ and R¹⁰ for each individual compound corresponds in each case to one line of Table B.

Table 44g Compounds of formula I.G in which o is 1, R⁷⁸ is 1″-CH₃, Y is C₂H₅ and the meaning for the combination of R¹, R², R³, R⁴, R⁹ and R¹⁰ for each individual compound corresponds in each case to one line of Table B.

Table 45g Compounds of formula I.G in which o is 1, R⁷⁸ is 1″-C₆H₅, Y is C₂H₅ and the meaning for the combination of R¹, R², R³, R⁴, R⁹ and R¹⁰ for each individual compound corresponds in each case to one line of Table B.

Table 46g Compounds of formula I.G in which o is 0, Y is CHF₂ and the meaning for the combination of R¹, R², R³, R⁴, R⁹ and R¹⁰ for each individual compound corresponds in each case to one line of Table B.

Table 47g Compounds of formula I.G in which o is 1, R⁷⁸ is 2″-F, Y is CHF₂ and the meaning for the combination of R¹, R², R³, R⁴, R⁹ and R¹⁰ for each individual compound corresponds in each case to one line of Table B.

Table 48g Compounds of formula I.G in which o is 1, R⁷⁸ is 2″-Cl, Y is CHF₂ and the meaning for the combination of R¹, R², R³, R⁴, R⁹ and R¹⁰ for each individual compound corresponds in each case to one line of Table B.

Table 49g Compounds of formula I.G in which o is 1, R⁷⁸ is 2″-Br, Y is CHF₂ and the meaning for the combination of R¹, R², R³, R⁴, R⁹ and R¹⁰ for each individual compound corresponds in each case to one line of Table B.

Table 50g Compounds of formula I.G in which o is 1, R⁷⁸ is 2″-CH₃, Y is CHF₂ and the meaning for the combination of R¹, R², R³, R⁴, R⁹ and R¹⁰ for each individual compound corresponds in each case to one line of Table B.

Table 51g Compounds of formula I.G in which o is 1, R⁷⁸ is 2″-OCH₃, Y is CHF₂ and the meaning for the combination of R¹, R², R³, R⁴, R⁹ and R¹⁰ for each individual compound corresponds in each case to one line of Table B.

Table 52g Compounds of formula I.G in which o is 1, R⁷⁸ is 2″-C₆H₅, Y is CHF₂ and the meaning for the combination of R¹, R², R³, R⁴, R⁹ and R¹⁰ for each individual compound corresponds in each case to one line of Table B.

Table 53g Compounds of formula I.G in which o is 1, R⁷⁸ is 1″-CH₃, Y is CHF₂ and the meaning for the combination of R¹, R², R³, R⁴, R⁹ and R¹⁰ for each individual compound corresponds in each case to one line of Table B.

Table 54g Compounds of formula I.G in which o is 1, R⁷⁸ is 1″-C₆H₅, Y is CHF₂ and the meaning for the combination of R¹, R², R³, R⁴, R⁹ and R¹⁰ for each individual compound corresponds in each case to one line of Table B.

Table 55g Compounds of formula I.G in which o is 0, Y is CN and the meaning for the combination of R¹, R², R³, R⁴, R⁹ and R¹⁰ for each individual compound corresponds in each case to one line of Table B.

Table 56g Compounds of formula I.G in which o is 1, R⁷⁸ is 2″-F, Y is CN and the meaning for the combination of R¹, R², R³, R⁴, R⁹ and R¹⁰ for each individual compound corresponds in each case to one line of Table B.

Table 57g Compounds of formula I.G in which o is 1, R⁷⁸ is 2″-Cl, Y is CN and the meaning for the combination of R¹, R², R³, R⁴, R⁹ and R¹⁰ for each individual compound corresponds in each case to one line of Table B.

Table 58g Compounds of formula I.G in which o is 1, R⁷⁸ is 2″-Br, Y is CN and the meaning for the combination of R¹, R², R³, R⁴, R⁹ and R¹⁰ for each individual compound corresponds in each case to one line of Table B.

Table 59g Compounds of formula I.G in which o is 1, R⁷⁸ is 2″-CH₃, Y is CN and the meaning for the combination of R¹, R², R³, R⁴, R⁹ and R¹⁰ for each individual compound corresponds in each case to one line of Table B.

Table 60g Compounds of formula I.G in which o is 1, R⁷⁸ is 2″-OCH₃, Y is CN and the meaning for the combination of R¹, R², R³, R⁴, R⁹ and R¹⁰ for each individual compound corresponds in each case to one line of Table B.

Table 61g Compounds of formula I.G in which o is 1, R⁷⁸ is 2″-C₆H₅, Y is CN and the meaning for the combination of R¹, R², R³, R⁴, R⁹ and R¹⁰ for each individual compound corresponds in each case to one line of Table B.

Table 62g Compounds of formula I.G in which o is 1, R⁷⁸ is 1″-CH₃, Y is CN and the meaning for the combination of R¹, R², R³, R⁴, R⁹ and R¹⁰ for each individual compound corresponds in each case to one line of Table B.

Table 63g Compounds of formula I.G in which o is 1, R⁷⁸ is 1″-C₆H₅, Y is CN and the meaning for the combination of R¹, R², R³, R⁴, R⁹ and R¹⁰ for each individual compound corresponds in each case to one line of Table B.

Table 64g Compounds of formula I.G in which o is 0, Y is OCHF₂ and the meaning for the combination of R¹, R², R³, R⁴, R⁹ and R¹⁰ for each individual compound corresponds in each case to one line of Table B.

Table 65g Compounds of formula I.G in which o is 1, R⁷⁸ is 2″-F, Y is OCHF₂ and the meaning for the combination of R¹, R², R³, R⁴, R⁹ and R¹⁰ for each individual compound corresponds in each case to one line of Table B.

Table 66g Compounds of formula I.G in which o is 1, R⁷⁸ is 2″-Cl, Y is OCHF₂ and the meaning for the combination of R¹, R², R³, R⁴, R⁹ and R¹⁰ for each individual compound corresponds in each case to one line of Table B.

Table 67g Compounds of formula I.G in which o is 1, R⁷⁸ is 2″-Br, Y is OCH F₂ and the meaning for the combination of R¹, R², R³, R⁴, R⁹ and R¹⁰ for each individual compound corresponds in each case to one line of Table B.

Table 68g Compounds of formula I.G in which o is 1, R⁷⁸ is 2″-CH₃, Y is OCHF₂ and the meaning for the combination of R¹, R², R³, R⁴, R⁹ and R¹⁰ for each individual compound corresponds in each case to one line of Table B.

Table 69g Compounds of formula I.G in which o is 1, R⁷⁸ is 2″-OCH₃, Y is OCH F₂ and the meaning for the combination of R¹, R², R³, R⁴, R⁹ and R¹⁰ for each individual compound corresponds in each case to one line of Table B.

Table 70g Compounds of formula I.G in which o is 1, R⁷⁸ is 2″-C₆H₅, Y is OCHF₂ and the meaning for the combination of R¹, R², R³, R⁴, R⁹ and R¹⁰ for each individual compound corresponds in each case to one line of Table B.

Table 71g Compounds of formula I.G in which o is 1, R⁷⁸ is 1″-CH₃, Y is OCHF₂ and the meaning for the combination of R¹, R², R³, R⁴, R⁹ and R¹⁰ for each individual compound corresponds in each case to one line of Table B.

Table 72g Compounds of formula I.G in which o is 1, R⁷⁸ is 1″-C₆H₅, Y is OCHF₂ and the meaning for the combination of R¹, R², R³, R⁴, R⁹ and R¹⁰ for each individual compound corresponds in each case to one line of Table B.

Table 73g Compounds of formula I.G in which o is 0, Y is Cl and the meaning for the combination of R¹, R², R³, R⁴, R⁹ and R¹⁰ for each individual compound corresponds in each case to one line of Table B.

Table 74g Compounds of formula I.G in which o is 1, R⁷⁸ is 2″-F, Y is Cl and the meaning for the combination of R¹, R², R³, R⁴, R⁹ and R¹⁰ for each individual compound corresponds in each case to one line of Table B.

Table 75g Compounds of formula I.G in which o is 1, R⁷⁸ is 2″-Cl, Y is Cl and the meaning for the combination of R¹, R², R³, R⁴, R⁹ and R¹⁰ for each individual compound corresponds in each case to one line of Table B.

Table 76g Compounds of formula I.G in which o is 1, R⁷⁸ is 2″-Br, Y is Cl and the meaning for the combination of R¹, R², R³, R⁴, R⁹ and R¹⁰ for each individual compound corresponds in each case to one line of Table B.

Table 77g Compounds of formula I.G in which o is 1, R⁷⁸ is 2″-CH₃, Y is Cl and the meaning for the combination of R¹, R², R³, R⁴, R⁹ and R¹⁰ for each individual compound corresponds in each case to one line of Table B.

Table 78g Compounds of formula I.G in which o is 1, R⁷⁸ is 2″-OCH₃, Y is Cl and the meaning for the combination of R¹, R², R³, R⁴, R⁹ and R¹⁰ for each individual compound corresponds in each case to one line of Table B.

Table 79g Compounds of formula I.G in which o is 1, R⁷⁸ is 2″-C₆H₅, Y is Cl and the meaning for the combination of R¹, R², R³, R⁴, R⁹ and R¹⁰ for each individual compound corresponds in each case to one line of Table B.

Table 80g Compounds of formula I.G in which o is 1, R⁷⁸ is 1″-CH₃, Y is Cl and the meaning for the combination of R¹, R², R³, R⁴, R⁹ and R¹⁰ for each individual compound corresponds in each case to one line of Table B.

Table 81g Compounds of formula I.G in which o is 1, R⁷⁸ is 1″-C₆H₅, Y is Cl and the meaning for the combination of R¹, R², R³, R⁴, R⁹ and R¹⁰ for each individual compound corresponds in each case to one line of Table B.

Table 82g Compounds of formula I.G in which o is 0, Y is F and the meaning for the combination of R¹, R², R³, R⁴, R⁹ and R¹⁰ for each individual compound corresponds in each case to one line of Table B.

Table 83g Compounds of formula I.G in which o is 1, R⁷⁸ is 2″-F, Y is F and the meaning for the combination of R¹, R², R³, R⁴, R⁹ and R¹⁰ for each individual compound corresponds in each case to one line of Table B.

Table 84g Compounds of formula I.G in which o is 1, R⁷⁸ is 2″-Cl, Y is F and the meaning for the combination of R¹, R², R³, R⁴, R⁹ and R¹⁰ for each individual compound corresponds in each case to one line of Table B.

Table 85g Compounds of formula I.G in which o is 1, R⁷⁸ is 2″-Br, Y is F and the meaning for the combination of R¹, R², R³, R⁴, R⁹ and R¹⁰ for each individual compound corresponds in each case to one line of Table B.

Table 86g Compounds of formula I.G in which o is 1, R⁷⁸ is 2″-CH₃, Y is F and the meaning for the combination of R¹, R², R³, R⁴, R⁹ and R¹⁰ for each individual compound corresponds in each case to one line of Table B.

Table 87g Compounds of formula I.G in which o is 1, R⁷⁸ is 2″-OCH₃, Y is F and the meaning for the combination of R¹, R², R³, R⁴, R⁹ and R¹⁰ for each individual compound corresponds in each case to one line of Table B.

Table 88g Compounds of formula I.G in which o is 1, R⁷⁸ is 2″-C₆H₅, Y is F and the meaning for the combination of R¹, R², R³, R⁴, R⁹ and R¹⁰ for each individual compound corresponds in each case to one line of Table B.

Table 89g Compounds of formula I.G in which o is 1, R⁷⁸ is 1″-CH₃, Y is F and the meaning for the combination of R¹, R², R³, R⁴, R⁹ and R¹⁰ for each individual compound corresponds in each case to one line of Table B.

Table 90g Compounds of formula I.G in which o is 1, R⁷⁸ is 1″-C₆H₅, Y is F and the meaning for the combination of R¹, R², R³, R⁴, R⁹ and R¹⁰ for each individual compound corresponds in each case to one line of Table B.

Table 1 h Compounds of formula I.H in which o is 0, Y is Br and the meaning for the combination of R¹, R², R³, R⁴, R⁹ and R¹⁰ for each individual compound corresponds in each case to one line of Table B.

Table 2h Compounds of formula I.H in which o is 1, R⁷⁸ is 1″-F, Y is Br and the meaning for the combination of R¹, R², R³, R⁴, R⁹ and R¹⁰ for each individual compound corresponds in each case to one line of Table B.

Table 3h Compounds of formula I.H in which o is 1, R⁷⁸ is 1″-Cl, Y is Br and the meaning for the combination of R¹, R², R³, R⁴, R⁹ and R¹⁰ for each individual compound corresponds in each case to one line of Table B.

Table 4h Compounds of formula I.H in which o is 1, R⁷⁸ is 1″-Br, Y is Br and the meaning for the combination of R¹, R², R³, R⁴, R⁹ and R¹⁰ for each individual compound corresponds in each case to one line of Table B.

Table 5h Compounds of formula I.H in which o is 1, R⁷⁸ is 1″-CH₃, Y is Br and the meaning for the combination of R¹, R², R³, R⁴, R⁹ and R¹⁰ for each individual compound corresponds in each case to one line of Table B.

Table 6h Compounds of formula I.H in which o is 1, R⁷⁸ is 1″-OCH₃, Y is Br and the meaning for the combination of R¹, R², R³, R⁴, R⁹ and R¹⁰ for each individual compound corresponds in each case to one line of Table B.

Table 7h Compounds of formula I.H in which o is 1, R⁷⁸ is 1″-OCHF₂, Y is Br and the meaning for the combination of R¹, R², R³, R⁴, R⁹ and R¹⁰ for each individual compound corresponds in each case to one line of Table B.

Table 8h Compounds of formula I.H in which o is 1, R⁷⁸ is 1″-C₆H₅, Y is Br and the meaning for the combination of R¹, R², R³, R⁴, R⁹ and R¹⁰ for each individual compound corresponds in each case to one line of Table B.

Table 9h Compounds of formula I.H in which o is 1, R⁷⁸ is 2″-CH₃, Y is Br and the meaning for the combination of R¹, R², R³, R⁴, R⁹ and R¹⁰ for each individual compound corresponds in each case to one line of Table B.

Table 10h Compounds of formula I.H in which o is 1, R⁷⁸ is 2″-C₆H₅, Y is Br and the meaning for the combination of R¹, R², R³, R⁴, R⁹ and R¹⁰ for each individual compound corresponds in each case to one line of Table B.

Table 11h Compounds of formula I.H in which o is 0, Y is CH₃ and the meaning for the combination of R¹, R², R³, R⁴, R⁹ and R¹⁰ for each individual compound corresponds in each case to one line of Table B.

Table 12h Compounds of formula I.H in which o is 1, R⁷⁸ is 1″-F, Y is CH₃ and the meaning for the combination of R¹, R², R³, R⁴, R⁹ and R¹⁰ for each individual compound corresponds in each case to one line of Table B.

Table 13h Compounds of formula I.H in which o is 1, R⁷⁸ is 1″-Cl, Y is CH₃ and the meaning for the combination of R¹, R², R³, R⁴, R⁹ and R¹⁰ for each individual compound corresponds in each case to one line of Table B.

Table 14h Compounds of formula I.H in which o is 1, R⁷⁸ is 1″-Br, Y is CH₃ and the meaning for the combination of R¹, R², R³, R⁴, R⁹ and R¹⁰ for each individual compound corresponds in each case to one line of Table B.

Table 15h Compounds of formula I.H in which o is 1, R⁷⁸ is 1″-CH₃, Y is CH₃ and the meaning for the combination of R¹, R², R³, R⁴, R⁹ and R¹⁰ for each individual compound corresponds in each case to one line of Table B.

Table 16h Compounds of formula I.H in which o is 1, R⁷⁸ is 1″-OCH₃, Y is CH₃ and the meaning for the combination of R¹, R², R³, R⁴, R⁹ and R¹⁰ for each individual compound corresponds in each case to one line of Table B.

Table 17h Compounds of formula I.H in which o is 1, R⁷⁸ is 1″-OCHF₂, Y is CH₃ and the meaning for the combination of R¹, R², R³, R⁴, R⁹ and R¹⁰ for each individual compound corresponds in each case to one line of Table B.

Table 18h Compounds of formula I.H in which o is 1, R⁷⁸ is 1″-C₆H₅, Y is CH₃ and the meaning for the combination of R¹, R², R³, R⁴, R⁹ and R¹⁰ for each individual compound corresponds in each case to one line of Table B.

Table 19h Compounds of formula I.H in which o is 1, R⁷⁸ is 2″-CH₃, Y is CH₃ and the meaning for the combination of R¹, R², R³, R⁴, R⁹ and R¹⁰ for each individual compound corresponds in each case to one line of Table B.

Table 20h Compounds of formula I.H in which o is 1, R⁷⁸ is 2″-C₆H₅, Y is CH₃ and the meaning for the combination of R¹, R², R³, R⁴, R⁹ and R¹⁰ for each individual compound corresponds in each case to one line of Table B.

Table 21h Compounds of formula I.H in which o is 0, Y is CF₃ and the meaning for the combination of R¹, R², R³, R⁴, R⁹ and R¹⁰ for each individual compound corresponds in each case to one line of Table B.

Table 22h Compounds of formula I.H in which o is 1, R⁷⁸ is 1″-F, Y is CF₃ and the meaning for the combination of R¹, R², R³, R⁴, R⁹ and R¹⁰ for each individual compound corresponds in each case to one line of Table B.

Table 23h Compounds of formula I.H in which o is 1, R⁷⁸ is 1″-Cl, Y is CF₃ and the meaning for the combination of R¹, R², R³, R⁴, R⁹ and R¹⁰ for each individual compound corresponds in each case to one line of Table B.

Table 24h Compounds of formula I.H in which o is 1, R⁷⁸ is 1″-Br, Y is CF₃ and the meaning for the combination of R¹, R², R³, R⁴, R⁹ and R¹⁰ for each individual compound corresponds in each case to one line of Table B.

Table 25h Compounds of formula I.H in which o is 1, R⁷⁸ is 1″-CH₃, Y is CF₃ and the meaning for the combination of R¹, R², R³, R⁴, R⁹ and R¹⁰ for each individual compound corresponds in each case to one line of Table B.

Table 26h Compounds of formula I.H in which o is 1, R⁷⁸ is 1″-OCH₃, Y is CF₃ and the meaning for the combination of R¹, R², R³, R⁴, R⁹ and R¹⁰ for each individual compound corresponds in each case to one line of Table B.

Table 27h Compounds of formula I.H in which o is 1, R⁷⁸ is 1″-OCHF₂, Y is CF₃ and the meaning for the combination of R¹, R², R³, R⁴, R⁹ and R¹⁰ for each individual compound corresponds in each case to one line of Table B.

Table 28h Compounds of formula I.H in which o is 1, R⁷⁸ is 1″-C₆H₅, Y is CF₃ and the meaning for the combination of R¹, R², R³, R⁴, R⁹ and R¹⁰ for each individual compound corresponds in each case to one line of Table B.

Table 29h Compounds of formula I.H in which o is 1, R⁷⁸ is 2″-CH₃, Y is CF₃ and the meaning for the combination of R¹, R², R³, R⁴, R⁹ and R¹⁰ for each individual compound corresponds in each case to one line of Table B.

Table 30h Compounds of formula I.H in which o is 1, R⁷⁸ is 2″-C₆H₅, Y is CF₃ and the meaning for the combination of R¹, R², R³, R⁴, R⁹ and R¹⁰ for each individual compound corresponds in each case to one line of Table B.

Table 31h Compounds of formula I.H in which o is 0, Y is CH(CH₃)₂ and the meaning for the combination of R¹, R², R³, R⁴, R⁹ and R¹⁰ for each individual compound corresponds in each case to one line of Table B.

Table 32h Compounds of formula I.H in which o is 1, R⁷⁸ is 1″-F, Y is CH(CH₃)₂ and the meaning for the combination of R¹, R², R³, R⁴, R⁹ and R¹⁰ for each individual compound corresponds in each case to one line of Table B.

Table 33h Compounds of formula I.H in which o is 1, R⁷⁸ is 1″-Cl, Y is CH(CH₃)₂ and the meaning for the combination of R¹, R², R³, R⁴, R⁹ and R¹⁰ for each individual compound corresponds in each case to one line of Table B.

Table 34h Compounds of formula I.H in which o is 1, R⁷⁸ is 1″-Br, Y is CH(CH₃)₂ and the meaning for the combination of R¹, R², R³, R⁴, R⁹ and R¹⁰ for each individual compound corresponds in each case to one line of Table B.

Table 35h Compounds of formula I.H in which o is 1, R⁷⁸ is 1″-CH₃, Y is CH(CH₃)₂ and the meaning for the combination of R¹, R², R³, R⁴, R⁹ and R¹⁰ for each individual compound corresponds in each case to one line of Table B.

Table 36h Compounds of formula I.H in which o is 1, R⁷⁸ is 1″-OCH₃, Y is CH(CH₃)₂ and the meaning for the combination of R¹, R², R³, R⁴, R⁹ and R¹⁰ for each individual compound corresponds in each case to one line of Table B.

Table 37h Compounds of formula I.H in which o is 1, R⁷⁸ is 1″-OCHF₂, Yis CH(CH₃)₂ and the meaning for the combination of R¹, R², R³, R⁴, R⁹ and R¹⁰ for each individual compound corresponds in each case to one line of Table B.

Table 38h Compounds of formula I.H in which o is 1, R⁷⁸ is 1″-C₆H₅, Yis CH(CH₃)₂ and the meaning for the combination of R¹, R², R³, R⁴, R⁹ and R¹⁰ for each individual compound corresponds in each case to one line of Table B.

Table 39h Compounds of formula I.H in which o is 1, R⁷⁸ is 2″-CH₃, Y is CH(CH₃)₂ and the meaning for the combination of R¹, R², R³, R⁴, R⁹ and R¹⁰ for each individual compound corresponds in each case to one line of Table B.

Table 40h Compounds of formula I.H in which o is 1, R⁷⁸ is 2″-C₆H₅, Yis CH(CH₃)₂ and the meaning for the combination of R¹, R², R³, R⁴, R⁹ and R¹⁰ for each individual compound corresponds in each case to one line of Table B.

Table 41h Compounds of formula I.H in which o is 0, Y is C₂H₅ and the meaning for the combination of R¹, R², R³, R⁴, R⁹ and R¹⁰ for each individual compound corresponds in each case to one line of Table B.

Table 42h Compounds of formula I.H in which o is 1, R⁷⁸ is 1″-F, Yis C₂H₅ and the meaning for the combination of R¹, R², R³, R⁴, R⁹ and R¹⁰ for each individual compound corresponds in each case to one line of Table B.

Table 43h Compounds of formula I.H in which o is 1, R⁷⁸ is 1″-Cl, Yis C₂H₅ and the meaning for the combination of R¹, R², R³, R⁴, R⁹ and R¹⁰ for each individual compound corresponds in each case to one line of Table B.

Table 44h Compounds of formula I.H in which o is 1, R⁷⁸ is 1″-Br, Yis C₂H₅ and the meaning for the combination of R¹, R², R³, R⁴, R⁹ and R¹⁰ for each individual compound corresponds in each case to one line of Table B.

Table 45h Compounds of formula I.H in which o is 1, R⁷⁸ is 1″-CH₃, Y is C₂H₅ and the meaning for the combination of R¹, R², R³, R⁴, R⁹ and R¹⁰ for each individual compound corresponds in each case to one line of Table B.

Table 46h Compounds of formula I.H in which o is 1, R⁷⁸ is 1″-OCH₃, Yis C₂H₅ and the meaning for the combination of R¹, R², R³, R⁴, R⁹ and R¹⁰ for each individual compound corresponds in each case to one line of Table B.

Table 47h Compounds of formula I.H in which o is 1, R⁷⁸ is 1″-OCHF₂, Yis C₂H₅ and the meaning for the combination of R¹, R², R³, R⁴, R⁹ and R¹⁰ for each individual compound corresponds in each case to one line of Table B.

Table 48h Compounds of formula I.H in which o is 1, R⁷⁸ is 1″-C₆H₅, Yis C₂H₅ and the meaning for the combination of R¹, R², R³, R⁴, R⁹ and R¹⁰ for each individual compound corresponds in each case to one line of Table B.

Table 49h Compounds of formula I.H in which o is 1, R⁷⁸ is 2″-CH₃, Y is C₂H₅ and the meaning for the combination of R¹, R², R³, R⁴, R⁹ and R¹⁰ for each individual compound corresponds in each case to one line of Table B.

Table 50h Compounds of formula I.H in which o is 1, R⁷⁸ is 2″-C₆H₅, Y is C₂H₅ and the meaning for the combination of R¹, R², R³, R⁴, R⁹ and R¹⁰ for each individual compound corresponds in each case to one line of Table B.

Table 51h Compounds of formula I.H in which o is 0, Y is CHF₂ and the meaning for the combination of R¹, R², R³, R⁴, R⁹ and R¹⁰ for each individual compound corresponds in each case to one line of Table B.

Table 52h Compounds of formula I.H in which o is 1, R⁷⁸ is 1″-F, Yis CHF₂ and the meaning for the combination of R¹, R², R³, R⁴, R⁹ and R¹⁰ for each individual compound corresponds in each case to one line of Table B.

Table 53h Compounds of formula I.H in which o is 1, R⁷⁸ is 1″-Cl, Yis CHF₂ and the meaning for the combination of R¹, R², R³, R⁴, R⁹ and R¹⁰ for each individual compound corresponds in each case to one line of Table B.

Table 54h Compounds of formula I.H in which o is 1, R⁷⁸ is 1″-Br, Yis CHF₂ and the meaning for the combination of R¹, R², R³, R⁴, R⁹ and R¹⁰ for each individual compound corresponds in each case to one line of Table B.

Table 55h Compounds of formula I.H in which o is 1, R⁷⁸ is 1″-CH₃, Y is CHF₂ and the meaning for the combination of R¹, R², R³, R⁴, R⁹ and R¹⁰ for each individual compound corresponds in each case to one line of Table B.

Table 56h Compounds of formula I.H in which o is 1, R⁷⁸ is 1″-OCH₃, Yis CHF₂ and the meaning for the combination of R¹, R², R³, R⁴, R⁹ and R¹⁰ for each individual compound corresponds in each case to one line of Table B.

Table 57h Compounds of formula I.H in which o is 1, R⁷⁸ is 1″-OCHF₂, Yis CHF₂ and the meaning for the combination of R¹, R², R³, R⁴, R⁹ and R¹⁰ for each individual compound corresponds in each case to one line of Table B.

Table 58h Compounds of formula I.H in which o is 1, R⁷⁸ is 1″-C₆H₅, Yis CHF₂ and the meaning for the combination of R¹, R², R³, R⁴, R⁹ and R¹⁰ for each individual compound corresponds in each case to one line of Table B.

Table 59h Compounds of formula I.H in which o is 1, R⁷⁸ is 2″-CH₃, Y is CHF₂ and the meaning for the combination of R¹, R², R³, R⁴, R⁹ and R¹⁰ for each individual compound corresponds in each case to one line of Table B.

Table 60h Compounds of formula I.H in which o is 1, R⁷⁸ is 2″-C₆H₅, Y is CHF₂ and the meaning for the combination of R¹, R², R³, R⁴, R⁹ and R¹⁰ for each individual compound corresponds in each case to one line of Table B.

Table 61h Compounds of formula I.H in which o is 0, Yis CN and the meaning for the combination of R¹, R², R³, R⁴, R⁹ and R¹⁰ for each individual compound corresponds in each case to one line of Table B.

Table 62h Compounds of formula I.H in which o is 1, R⁷⁸ is 1″-F, Yis CN and the meaning for the combination of R¹, R², R³, R⁴, R⁹ and R¹⁰ for each individual compound corresponds in each case to one line of Table B.

Table 63h Compounds of formula I.H in which o is 1, R⁷⁸ is 1″-Cl, Yis CN and the meaning for the combination of R¹, R², R³, R⁴, R⁹ and R¹⁰ for each individual compound corresponds in each case to one line of Table B.

Table 64h Compounds of formula I.H in which o is 1, R⁷⁸ is 1″-Br, Yis CN and the meaning for the combination of R¹, R², R³, R⁴, R⁹ and R¹⁰ for each individual compound corresponds in each case to one line of Table B.

Table 65h Compounds of formula I.H in which o is 1, R⁷⁸ is 1″-CH₃, Y is CN and the meaning for the combination of R¹, R², R³, R⁴, R⁹ and R¹⁰ for each individual compound corresponds in each case to one line of Table B.

Table 66h Compounds of formula I.H in which o is 1, R⁷⁸ is 1″-OCH₃, Yis CN and the meaning for the combination of R¹, R², R³, R⁴, R⁹ and R¹⁰ for each individual compound corresponds in each case to one line of Table B.

Table 67h Compounds of formula I.H in which o is 1, R⁷⁸ is 1″-OCHF₂, Yis CN and the meaning for the combination of R¹, R², R³, R⁴, R⁹ and R¹⁰ for each individual compound corresponds in each case to one line of Table B.

Table 68h Compounds of formula I.H in which o is 1, R⁷⁸ is 1″-C₆H₅, Yis CN and the meaning for the combination of R¹, R², R³, R⁴, R⁹ and R¹⁰ for each individual compound corresponds in each case to one line of Table B.

Table 69h Compounds of formula I.H in which o is 1, R⁷⁸ is 2″-CH₃, Y is CN and the meaning for the combination of R¹, R², R³, R⁴, R⁹ and R¹⁰ for each individual compound corresponds in each case to one line of Table B.

Table 70h Compounds of formula I.H in which o is 1, R⁷⁸ is 2″-C₆H₅, Y is CN and the meaning for the combination of R¹, R², R³, R⁴, R⁹ and R¹⁰ for each individual compound corresponds in each case to one line of Table B.

Table 71h Compounds of formula I.H in which o is 0, Yis OCHF₂ and the meaning for the combination of R¹, R², R³, R⁴, R⁹ and R¹⁰ for each individual compound corresponds in each case to one line of Table B.

Table 72h Compounds of formula I.H in which o is 1, R⁷⁸ is 1″-F, Yis OCHF₂ and the meaning for the combination of R¹, R², R³, R⁴, R⁹ and R¹⁰ for each individual compound corresponds in each case to one line of Table B.

Table 73h Compounds of formula I.H in which o is 1, R⁷⁸ is 1″-Cl, Yis OCHF₂ and the meaning for the combination of R¹, R², R³, R⁴, R⁹ and R¹⁰ for each individual compound corresponds in each case to one line of Table B.

Table 74h Compounds of formula I.H in which o is 1, R⁷⁸ is 1″-Br, Yis OCHF₂ and the meaning for the combination of R¹, R², R³, R⁴, R⁹ and R¹⁰ for each individual compound corresponds in each case to one line of Table B.

Table 75h Compounds of formula I.H in which o is 1, R⁷⁸ is 1″-CH₃, Y is OCHF₂ and the meaning for the combination of R¹, R², R³, R⁴, R⁹ and R¹⁰ for each individual compound corresponds in each case to one line of Table B.

Table 76h Compounds of formula I.H in which o is 1, R⁷⁸ is 1″-OCH₃, Yis OCHF₂ and the meaning for the combination of R¹, R², R³, R⁴, R⁹ and R¹⁰ for each individual compound corresponds in each case to one line of Table B.

Table 77h Compounds of formula I.H in which o is 1, R⁷⁸ is 1″-OCHF₂, Yis OCHF₂ and the meaning for the combination of R¹, R², R³, R⁴, R⁹ and R¹⁰ for each individual compound corresponds in each case to one line of Table B.

Table 78h Compounds of formula I.H in which o is 1, R⁷⁸ is 1″-C₆H₅, Yis OCHF₂ and the meaning for the combination of R¹, R², R³, R⁴, R⁹ and R¹⁰ for each individual compound corresponds in each case to one line of Table B.

Table 79h Compounds of formula I.H in which o is 1, R⁷⁸ is 2″-CH₃, Y is OCHF₂ and the meaning for the combination of R¹, R², R³, R⁴, R⁹ and R¹⁰ for each individual compound corresponds in each case to one line of Table B.

Table 80h Compounds of formula I.H in which o is 1, R⁷⁸ is 2″-C₆H₅, Y is OCHF₂ and the meaning for the combination of R¹, R², R³, R⁴, R⁹ and R¹⁰ for each individual compound corresponds in each case to one line of Table B.

Table 81h Compounds of formula I.H in which o is 0, Y is Cl and the meaning for the combination of R¹, R², R³, R⁴, R⁹ and R¹⁰ for each individual compound corresponds in each case to one line of Table B.

Table 82h Compounds of formula I.H in which o is 1, R⁷⁸ is 1″-F, Y is Cl and the meaning for the combination of R¹, R², R³, R⁴, R⁹ and R¹⁰ for each individual compound corresponds in each case to one line of Table B.

Table 83h Compounds of formula I.H in which o is 1, R⁷⁸ is 1″-Cl, Y is Cl and the meaning for the combination of R¹, R², R³, R⁴, R⁹ and R¹⁰ for each individual compound corresponds in each case to one line of Table B.

Table 84h Compounds of formula I.H in which o is 1, R⁷⁸ is 1″-Br, Y is Cl and the meaning for the combination of R¹, R², R³, R⁴, R⁹ and R¹⁰ for each individual compound corresponds in each case to one line of Table B.

Table 85h Compounds of formula I.H in which o is 1, R⁷⁸ is 1″-CH₃, Y is Cl and the meaning for the combination of R¹, R², R³, R⁴, R⁹ and R¹⁰ for each individual compound corresponds in each case to one line of Table B.

Table 86h Compounds of formula I.H in which o is 1, R⁷⁸ is 1″-OCH₃, Y is Cl and the meaning for the combination of R¹, R², R³, R⁴, R⁹ and R¹⁰ for each individual compound corresponds in each case to one line of Table B.

Table 87h Compounds of formula I.H in which o is 1, R⁷⁸ is 1″-OCHF₂, Y is Cl and the meaning for the combination of R¹, R², R³, R⁴, R⁹ and R¹⁰ for each individual compound corresponds in each case to one line of Table B.

Table 88h Compounds of formula I.H in which o is 1, R⁷⁸ is 1″-C₆H₅, Y is Cl and the meaning for the combination of R¹, R², R³, R⁴, R⁹ and R¹⁰ for each individual compound corresponds in each case to one line of Table B.

Table 89h Compounds of formula I.H in which o is 1, R⁷⁸ is 2″-CH₃, Y is Cl and the meaning for the combination of R¹, R², R³, R⁴, R⁹ and R¹⁰ for each individual compound corresponds in each case to one line of Table B.

Table 90h Compounds of formula I.H in which o is 1, R⁷⁸ is 2″-C₆H₅, Y is Cl and the meaning for the combination of R¹, R², R³, R⁴, R⁹ and R¹⁰ for each individual compound corresponds in each case to one line of Table B.

Table 91h Compounds of formula I.H in which o is 0, Y is F and the meaning for the combination of R¹, R², R³, R⁴, R⁹ and R¹⁰ for each individual compound corresponds in each case to one line of Table B.

Table 92h Compounds of formula I.H in which o is 1, R⁷⁸ is 1″-F, Y is F and the meaning for the combination of R¹, R², R³, R⁴, R⁹ and R¹⁰ for each individual compound corresponds in each case to one line of Table B.

Table 93h Compounds of formula I.H in which o is 1, R⁷⁸ is 1″-Cl, Y is F and the meaning for the combination of R¹, R², R³, R⁴, R⁹ and R¹⁰ for each individual compound corresponds in each case to one line of Table B.

Table 94h Compounds of formula I.H in which o is 1, R⁷⁸ is 1″-Br, Y is F and the meaning for the combination of R¹, R², R³, R⁴, R⁹ and R¹⁰ for each individual compound corresponds in each case to one line of Table B.

Table 95h Compounds of formula I.H in which o is 1, R⁷⁸ is 1″-CH₃, Y is F and the meaning for the combination of R¹, R², R³, R⁴, R⁹ and R¹⁰ for each individual compound corresponds in each case to one line of Table B.

Table 96h Compounds of formula I.H in which o is 1, R⁷⁸ is 1″-OCH₃, Y is F and the meaning for the combination of R¹, R², R³, R⁴, R⁹ and R¹⁰ for each individual compound corresponds in each case to one line of Table B.

Table 97h Compounds of formula I.H in which o is 1, R⁷⁸ is 1″-OCHF₂, Y is F and the meaning for the combination of R¹, R², R³, R⁴, R⁹ and R¹⁰ for each individual compound corresponds in each case to one line of Table B.

Table 98h Compounds of formula I.H in which o is 1, R⁷⁸ is 1″-C₆H₅, Y is F and the meaning for the combination of R¹, R², R³, R⁴, R⁹ and R¹⁰ for each individual compound corresponds in each case to one line of Table B.

Table 99h Compounds of formula I.H in which o is 1, R⁷⁸ is 2″-CH₃, Y is F and the meaning for the combination of R¹, R², R³, R⁴, R⁹ and R¹⁰ for each individual compound corresponds in each case to one line of Table B.

Table 100h Compounds of formula I.H in which o is 1, R⁷⁸ is 2″-C₆H₅, Y is F and the meaning for the combination of R¹, R², R³, R⁴, R⁹ and R¹⁰ for each individual compound corresponds in each case to one line of Table B.

Table 1i Compounds of formula I.I in which o is 0, R¹² is H and the meaning for the combination of R¹, R², R³, R⁴, R⁹ and R¹⁰ for each individual compound corresponds in each case to one line of Table B.

Table 2i Compounds of formula I.I in which o is 1, R⁷⁸ is 2″-F, R¹² is H and the meaning for the combination of R¹, R², R³, R⁴, R⁹ and R¹⁰ for each individual compound corresponds in each case to one line of Table B.

Table 3i Compounds of formula I.I in which o is 1, R⁷⁸ is 2″-C1, R¹² is H and the meaning for the combination of R¹, R², R³, R⁴, R⁹ and R¹⁰ for each individual compound corresponds in each case to one line of Table B.

Table 4i Compounds of formula I.I in which o is 1, R⁷⁸ is 2″-Br, R¹² is H and the meaning for the combination of R¹, R², R³, R⁴, R⁹ and R¹⁰ for each individual compound corresponds in each case to one line of Table B.

Table 5i Compounds of formula I.I in which o is 1, R⁷⁸ is 2″-CH₃, R¹² is H and the meaning for the combination of R¹, R², R³, R⁴, R⁹ and R^{10 6} for each individual compound corresponds in each case to one line of Table B.

Table 6i Compounds of formula I.I in which o is 1, R⁷⁸ is 2″-OCH₃, R¹² is H and the meaning for the combination of R¹, R², R³, R⁴, R⁹ and R¹⁰ for each individual compound corresponds in each case to one line of Table B.

Table 7i Compounds of formula I.I in which o is 1, R⁷⁸ is 2″-OCHF₂, R¹² is H and the meaning for the combination of R¹, R², R³, R⁴, R⁹ and R¹⁰ for each individual compound corresponds in each case to one line of Table B.

Table 8i Compounds of formula I.I in which o is 1, R⁷⁸ is 2″-C₆H₅, R¹² is H and the meaning for the combination of R¹, R², R³, R⁴, R⁹ and R¹⁰ for each individual compound corresponds in each case to one line of Table B.

Table 9i Compounds of formula I.I in which o is 1, R⁷⁸ is 3″-F, R¹² is H and the meaning for the combination of R¹, R², R³, R⁴, R⁹ and R¹⁰ for each individual compound corresponds in each case to one line of Table B.

Table 10i Compounds of formula I.I in which o is 1, R⁷⁸ is 3″-C1, R¹² is H and the meaning for the combination of R¹, R², R³, R⁴, R⁵ R⁶, R⁹ and R¹⁰ for each individual compound corresponds in each case to one line of Table B.

Table 11i Compounds of formula I.I in which o is 1, R⁷⁸ is 3″-Br, R¹² is H and the meaning for the combination of R¹, R², R³, R⁴, R⁹ and R¹⁰ for each individual compound corresponds in each case to one line of Table B.

Table 12i Compounds of formula I.I in which o is 1, R⁷⁸ is 3″-CH₃, R¹² is H and the meaning for the combination of R¹, R², R³, R⁴, R⁹ and R¹⁰ for each individual compound corresponds in each case to one line of Table B.

Table 13i Compounds of formula I.I in which o is 1, R⁷⁸ is 3″-OCH₃, R¹² is H and the meaning for the combination of R¹, R², R³, R⁴, R⁹ and R¹⁰ for each individual compound corresponds in each case to one line of Table B.

Table 14i Compounds of formula I.I in which o is 1, R⁷⁸ is 3″-OCHF₂, R¹² is H and the meaning for the combination of R¹, R², R³, R⁴, R⁹ and R¹⁰ for each individual compound corresponds in each case to one line of Table B.

Table 15i Compounds of formula I.I in which o is 1, R⁷⁸ is 3″-C₆H₅, R¹² is H and the meaning for the combination of R¹, R², R³, R⁴, R⁹ and R¹⁰ for each individual compound corresponds in each case to one line of Table B.

Table 16i Compounds of formula I.I in which o is 0, R¹² is CH₃ and the meaning for the combination of R¹, R², R³, R⁴, R⁹ and R¹⁰ for each individual compound corresponds in each case to one line of Table B.

Table 17i Compounds of formula I.I in which o is 1, R⁷⁸ is 2″-F, R¹² is CH₃ and the meaning for the combination of R¹, R², R³, R⁴, R⁹ and R¹⁰ for each individual compound corresponds in each case to one line of Table B.

Table 18i Compounds of formula I.I in which o is 1, R⁷⁸ is 2″-C1, R¹² is CH₃ and the meaning for the combination of R¹, R², R³, R⁴, R⁹ and R¹⁰ for each individual compound corresponds in each case to one line of Table B.

Table 19i Compounds of formula I.I in which o is 1, R⁷⁸ is 2″-Br, R¹² is CH₃ and the meaning for the combination of R¹, R², R³, R⁴, R⁹ and R¹⁰ for each individual compound corresponds in each case to one line of Table B.

Table 20i Compounds of formula I.I in which o is 1, R⁷⁸ is 2″-CH₃, R¹² is CH₃ and the meaning for the combination of R¹, R², R³, R⁴, R⁹ and R¹⁰ for each individual compound corresponds in each case to one line of Table B.

Table 21i Compounds of formula I.I in which o is 1, R⁷⁸ is 2″-OCH₃, R¹² is CH₃ and the meaning for the combination of R¹, R², R³, R⁴, R⁹ and R¹⁰ for each individual compound corresponds in each case to one line of Table B.

Table 22i Compounds of formula I.I in which o is 1, R⁷⁸ is 2″-OCHF₂, R¹² is CH₃ and the meaning for the combination of R¹, R², R³, R⁴, R⁹ and R¹⁰ for each individual compound corresponds in each case to one line of Table B.

Table 23i Compounds of formula I.I in which o is 1, R⁷⁸ is 2″-C₆H₅, R¹² is CH₃ and the meaning for the combination of R¹, R², R³, R⁴, R⁹ and R¹⁰ for each individual compound corresponds in each case to one line of Table B.

Table 24i Compounds of formula I.I in which o is 1, R⁷⁸ is 3″-F, R¹² is CH₃ and the meaning for the combination of R¹, R², R³, R⁴, R⁹ and R¹⁰ for each individual compound corresponds in each case to one line of Table B.

Table 25i Compounds of formula I.I in which o is 1, R⁷⁸ is 3″-C1, R¹² is CH₃ and the meaning for the combination of R¹, R², R³, R⁴, R⁵ R⁶ R⁹ and R¹⁰ for each individual compound corresponds in each case to one line of Table B.

Table 26i Compounds of formula I.I in which o is 1, R⁷⁸ is 3″-Br, R¹² is CH₃ and the mean-ing for the combination of R¹, R², R³, R⁴, R⁹ and R¹⁰ for each individual compound corresponds in each case to one line of Table B.

Table 27i Compounds of formula I.I in which o is 1, R⁷⁸ is 3″-CH₃, R¹² is CH₃ and the meaning for the combination of R¹, R², R³, R⁴, R⁹ and R¹⁰ for each individual compound corresponds in each case to one line of Table B.

Table 28i Compounds of formula I.I in which o is 1, R⁷⁸ is 3″-OCH₃, R¹² is CH₃ and the meaning for the combination of R¹, R², R³, R⁴, R⁹ and R¹⁰ for each individual compound corresponds in each case to one line of Table B.

Table 29i Compounds of formula I.I in which o is 1, R⁷⁸ is 3″-OCHF₂, R¹² is CH₃ and the meaning for the combination of R¹, R², R³, R⁴, R⁹ and R¹⁰ for each individual compound corresponds in each case to one line of Table B.

Table 30i Compounds of formula I.I in which o is 1, R⁷⁸ is 3″-C₆H₅, R¹² is CH₃ and the meaning for the combination of R¹, R², R³, R⁴, R⁹ and R¹⁰ for each individual compound corresponds in each case to one line of Table B.

Table 31i Compounds of formula I.I in which o is 0, R¹² is CF₃ and the meaning for the combination of R¹, R², R³, R⁴, R⁹ and R¹⁰ for each individual compound corresponds in each case to one line of Table B.

Table 32i Compounds of formula I.I in which o is 1, R⁷⁸ is 2″-F, R¹² is CF₃ and the meaning for the combination of R¹, R², R³, R⁴, R⁹ and R¹⁰ for each individual compound corresponds in each case to one line of Table B.

Table 33i Compounds of formula I.I in which o is 1, R⁷⁸ is 2″-C1, R¹² is CF₃ and the meaning for the combination of R¹, R², R³, R⁴, R⁹ and R¹⁰ for each individual compound corresponds in each case to one line of Table B.

Table 34i Compounds of formula I.I in which o is 1, R⁷⁸ is 2″-Br, R¹² is CF₃ and the meaning for the combination of R¹, R², R³, R⁴, R⁹ and R¹⁰ for each individual compound corresponds in each case to one line of Table B.

Table 35i Compounds of formula I.I in which o is 1, R⁷⁸ is 2″-CH₃, R¹² is CF₃ and the meaning for the combination of R¹, R², R³, R⁴, R⁹ and R¹⁰ for each individual compound corresponds in each case to one line of Table B.

Table 36i Compounds of formula I.I in which o is 1, R⁷⁸ is 2″-OCH₃, R¹² is CF₃ and the meaning for the combination of R¹, R², R³, R⁴, R⁹ and R¹⁰ for each individual compound corresponds in each case to one line of Table B.

Table 37i Compounds of formula I.I in which o is 1, R⁷⁸ is 2″-OCHF₂, R¹² is CF₃ and the meaning for the combination of R¹, R², R³, R⁴, R⁹ and R¹⁰ for each individual compound corresponds in each case to one line of Table B.

Table 38i Compounds of formula I.I in which o is 1, R⁷⁸ is 2″-C₆H₅, R¹² is CF₃ and the meaning for the combination of R¹, R², R³, R⁴, R⁹ and R¹⁰ for each individual compound corresponds in each case to one line of Table B.

Table 39i Compounds of formula I.I in which o is 1, R⁷⁸ is 3″-F, R¹² is CF₃ and the meaning for the combination of R¹, R², R³, R⁴, R⁹ and R¹⁰ for each individual compound corresponds in each case to one line of Table B.

Table 40i Compounds of formula I.I in which o is 1, R⁷⁸ is 3″-Cl, R¹² is CF₃ and the meaning for the combination of R¹, R², R³, R⁴, R⁵ R⁶, R⁹ and R¹⁰ for each individual compound corresponds in each case to one line of Table B.

Table 41i Compounds of formula I.I in which o is 1, R⁷⁸ is 3″-Br, R¹² is CF₃ and the meaning for the combination of R¹, R², R³, R⁴, R⁹ and R¹⁰ for each individual compound corresponds in each case to one line of Table B.

Table 42i Compounds of formula I.I in which o is 1, R⁷⁸ is 3″-CH₃, R¹² is CF₃ and the meaning for the combination of R¹, R², R³, R⁴, R⁹ and R¹⁰ for each individual compound corresponds in each case to one line of Table B.

Table 43i Compounds of formula I.I in which o is 1, R⁷⁸ is 3″-OCH₃, R¹² is CF₃ and the meaning for the combination of R¹, R², R³, R⁴, R⁹ and R¹⁰ for each individual compound corresponds in each case to one line of Table B.

Table 44i Compounds of formula I.I in which o is 1, R⁷⁸ is 3″-OCHF₂, R¹² is CF₃ and the meaning for the combination of R¹, R², R³, R⁴, R⁹ and R¹⁰ for each individual compound corresponds in each case to one line of Table B.

Table 45i Compounds of formula I.I in which o is 1, R⁷⁸ is 3″-C₆H₅, R¹² is CF₃ and the meaning for the combination of R¹, R², R³, R⁴, R⁹ and R¹⁰ for each individual compound corresponds in each case to one line of Table B.

Table 46i Compounds of formula I.I in which o is 0, R¹² is CH(CH₃)₂ and the meaning for the combination of R¹, R², R³, R⁴, R⁹ and R¹⁰ for each individual compound corresponds in each case to one line of Table B.

Table 47i Compounds of formula I.I in which o is 1, R⁷⁸ is 2″-F, R¹² is CH(CH₃)₂ and the meaning for the combination of R¹, R², R³, R⁴, R⁹ and R¹⁰ for each individual compound corresponds in each case to one line of Table B.

Table 48i Compounds of formula I.I in which o is 1, R⁷⁸ is 2″-C1, R¹² is CH(CH₃)₂ and the meaning for the combination of R¹, R², R³, R⁴, R⁹ and R¹⁰ for each individual compound corresponds in each case to one line of Table B.

Table 49i Compounds of formula I.I in which o is 1, R⁷⁸ is 2″-Br, R¹² is CH(CH₃)₂ and the meaning for the combination of R¹, R², R³, R⁴, R⁹ and R¹⁰ for each individual compound corresponds in each case to one line of Table B.

Table 50i Compounds of formula I.I in which o is 1, R⁷⁸ is 2″-CH₃, R¹² is CH(CH₃)₂ and the meaning for the combination of R¹, R², R³, R⁴, R⁹ and R^{10 6} for each individual compound corresponds in each case to one line of Table B.

Table 51i Compounds of formula I.I in which o is 1, R⁷⁸ is 2″-OCH₃, R¹² is CH(CH₃)₂ and the meaning for the combination of R¹, R², R³, R⁴, R⁹ and R¹⁰ for each individual compound corresponds in each case to one line of Table B.

Table 52i Compounds of formula I.I in which o is 1, R⁷⁸ is 2″-OCHF₂, R¹² is CH(CH₃)₂ and the meaning for the combination of R¹, R², R³, R⁴, R⁹ and R¹⁰ for each individual compound corresponds in each case to one line of Table B.

Table 53i Compounds of formula I.I in which o is 1, R⁷⁸ is 2″-C₆H₅, R¹² is CH(CH₃)₂ and the meaning for the combination of R¹, R², R³, R⁴, R⁹ and R¹⁰ for each individual compound corresponds in each case to one line of Table B.

Table 54i Compounds of formula I.I in which o is 1, R⁷⁸ is 3″-F, R¹² is CH(CH₃)₂ and the meaning for the combination of R¹, R², R³, R⁴, R⁹ and R¹⁰ for each individual compound corresponds in each case to one line of Table B.

le 55i Compounds of formula I.I in which o is 1, R⁷⁸ is 3″-C1, R¹² is CH(CH₃)₂ and the meaning for the combination of R¹, R², R³, R⁴, R⁵ R⁶, R⁹ and R¹⁰ for each individual compound corresponds in each case to one line of Table B.

le 56i Compounds of formula I.I in which o is 1, R⁷⁸ is 3″-Br, R¹² is CH(CH₃)₂ and the meaning for the combination of R¹, R², R³, R⁴, R⁹ and R¹⁰ for each individual compound corre-sponds in each case to one line of Table B.

le 57i Compounds of formula I.I in which o is 1, R⁷⁸ is 3″-CH₃, R¹² is CH(CH₃)₂ and the meaning for the combination of R¹, R², R³, R⁴, R⁹ and R¹⁰ for each individual compound corresponds in each case to one line of Table B.

Table 58i Compounds of formula I.I in which o is 1, R⁷⁸ is 3″-OCH₃, R¹² is CH(CH₃)₂ and the meaning for the combination of R¹, R², R³, R⁴, R⁹ and R¹⁰ for each individual compound corresponds in each case to one line of Table B.

Table 59i Compounds of formula I.I in which o is 1, R⁷⁸ is 3″-OCHF₂, R¹² is CH(CH₃)₂ and the meaning for the combination of R¹, R², R³, R⁴, R⁹ and R¹⁰ for each individual compound corresponds in each case to one line of Table B.

Table 60i Compounds of formula I.I in which o is 1, R⁷⁸ is 3″-C₆H₅, R¹² is CH(CH₃)₂ and the meaning for the combination of R¹, R², R³, R⁴, R⁹ and R¹⁰ for each individual compound corresponds in each case to one line of Table B.

Table 61i Compounds of formula I.I in which o is 0, R¹² is C₂H₅ and the meaning for the combination of R¹, R², R³, R⁴, R⁹ and R¹⁰ for each individual compound corresponds in each case to one line of Table B.

Table 62i Compounds of formula I.I in which o is 1, R⁷⁸ is 2″-F, R¹² is C₂H₅ and the meaning for the combination of R¹, R², R³, R⁴, R⁹ and R¹⁰ for each individual compound corresponds in each case to one line of Table B.

Table 63i Compounds of formula I.I in which o is 1, R⁷⁸ is 2″-C1, R¹² is C₂H₅ and the meaning for the combination of R¹, R², R³, R⁴, R⁹ and R¹⁰ for each individual compound corresponds in each case to one line of Table B.

Table 64i Compounds of formula I.I in which o is 1, R⁷⁸ is 2″-Br, R¹² is C₂H₅ and the meaning for the combination of R¹, R², R³, R⁴, R⁹ and R¹⁰ for each individual compound corresponds in each case to one line of Table B.

Table 65i Compounds of formula I.I in which o is 1, R⁷⁸ is 2″-CH₃, R¹² is C₂H₅ and the meaning for the combination of R¹, R², R³, R⁴, R⁹ and R^{10 6} for each individual compound corresponds in each case to one line of Table B.

Table 66i Compounds of formula I.I in which o is 1, R⁷⁸ is 2″-OCH₃, R¹² is C₂H₅ and the meaning for the combination of R¹, R², R³, R⁴, R⁹ and R¹⁰ for each individual compound corresponds in each case to one line of Table B.

Table 67i Compounds of formula I.I in which o is 1, R⁷⁸ is 2″-OCHF₂, R¹² is C₂H₅ and the meaning for the combination of R¹, R², R³, R⁴, R⁹ and R¹⁰ for each individual compound corresponds in each case to one line of Table B.

Table 68i Compounds of formula I.I in which o is 1, R⁷⁸ is 2″-C₆H₅, R¹² is C₂H₅ and the meaning for the combination of R¹, R², R³, R⁴, R⁹ and R¹⁰ for each individual compound corresponds in each case to one line of Table B.

Table 69i Compounds of formula I.I in which o is 1, R⁷⁸ is 3″-F, R¹² is C₂H₅ and the meaning for the combination of R¹, R², R³, R⁴, R⁹ and R¹⁰ for each individual compound corresponds in each case to one line of Table B.

Table 70i Compounds of formula I.I in which o is 1, R⁷⁸ is 3″-C1, R¹² is C₂H₅ and the meaning for the combination of R¹, R², R³, R4, R5 R6, R⁹ and R¹⁰ for each individual compound corresponds in each case to one line of Table B.

Table 71i Compounds of formula I.I in which o is 1, R⁷⁸ is 3″-Br, R¹² is C₂H₅ and the meaning for the combination of R¹, R², R³, R⁴, R⁹ and R¹⁰ for each individual compound corresponds in each case to one line of Table B.

Table 72i Compounds of formula I.I in which o is 1, R⁷⁸ is 3″-CH₃, R¹² is C₂H₅ and the meaning for the combination of R¹, R², R³, R⁴, R⁹ and R¹⁰ for each individual compound corresponds in each case to one line of Table B.

Table 73i Compounds of formula I.I in which o is 1, R⁷⁸ is 3″-OCH₃, R¹² is C₂H₅ and the meaning for the combination of R¹, R², R³, R⁴, R⁹ and R¹⁰ for each individual compound corresponds in each case to one line of Table B.

Table 74i Compounds of formula I.I in which o is 1, R⁷⁸ is 3″-OCHF₂, R¹² is C₂H₅ and the meaning for the combination of R¹, R², R³, R⁴, R⁹ and R¹⁰ for each individual compound corresponds in each case to one line of Table B.

Table 75i Compounds of formula I.I in which o is 1, R⁷⁸ is 3″-C6H₅, R¹² is C₂H₅ and the meaning for the combination of R¹, R², R³, R⁴, R⁹ and R¹⁰ for each individual compound corresponds in each case to one line of Table B.

Table 76i Compounds of formula I.I in which o is 0, R¹² is CHF₂ and the meaning for the combination of R¹, R², R³, R⁴, R⁹ and R¹⁰ for each individual compound corresponds in each case to one line of Table B.

Table 77i Compounds of formula I.I in which o is 1, R⁷⁸ is 2″-F, R¹² is CHF₂ and the meaning for the combination of R¹, R², R³, R⁴, R⁹ and R¹⁰ for each individual compound corresponds in each case to one line of Table B.

Table 78i Compounds of formula I.I in which o is 1, R⁷⁸ is 2″-C1, R¹² is CHF₂ and the meaning for the combination of R¹, R², R³, R⁴, R⁹ and R¹⁰ for each individual compound corresponds in each case to one line of Table B.

Table 79i Compounds of formula I.I in which o is 1, R⁷⁸ is 2″-Br, R¹² is CHF₂ and the meaning for the combination of R¹, R², R³, R⁴, R⁹ and R¹⁰ for each individual compound corresponds in each case to one line of Table B.

Table 80i Compounds of formula I.I in which o is 1, R⁷⁸ is 2″-CH₃, R¹² is CHF₂ and the meaning for the combination of R¹, R², R³, R⁴, R⁹ and R¹⁰ ⁶ for each individual compound corresponds in each case to one line of Table B.

Table 81i Compounds of formula I.I in which o is 1, R⁷⁸ is 2″-OCH3, R¹² is CHF₂ and the meaning for the combination of R¹, R², R³, R⁴, R⁹ and R¹⁰ for each individual compound corresponds in each case to one line of Table B.

Table 82i Compounds of formula I.I in which o is 1, R⁷⁸ is 2″-OCHF₂, R¹² is CHF₂ and the meaning for the combination of R¹, R², R³, R⁴, R⁹ and R¹⁰ for each individual compound corresponds in each case to one line of Table B.

Table 83i Compounds of formula I.I in which o is 1, R⁷⁸ is 2″-C₆H₅, R¹² is CHF₂ and the meaning for the combination of R¹, R², R³, R⁴, R⁹ and R¹⁰ for each individual compound corresponds in each case to one line of Table B.

Table 84i Compounds of formula I.I in which o is 1, R⁷⁸ is 3″-F, R¹² is CHF₂ and the meaning for the combination of R¹, R², R³, R⁴, R⁹ and R¹⁰ for each individual compound corresponds in each case to one line of Table B.

Table 85i Compounds of formula I.I in which o is 1, R⁷⁸ is 3″-Cl, R¹² is CHF₂ and the meaning for the combination of R¹, R², R³, R⁴, R⁵ R⁶, R⁹ and R¹⁰for each individual compound corresponds in each case to one line of Table B.

Table 86i Compounds of formula I.I in which o is 1, R⁷⁸ is 3″-Br, R¹² is CHF₂ and the meaning for the combination of R¹, R², R³, R⁴, R⁹ and R¹⁰ for each individual compound corresponds in each case to one line of Table B.

Table 87i Compounds of formula I.I in which o is 1, R⁷⁸ is 3″-CH₃, R¹² is CHF₂ and the meaning for the combination of R¹, R², R³, R⁴, R⁹ and R¹⁰ for each individual compound corresponds in each case to one line of Table B.

Table 88i Compounds of formula I.I in which o is 1, R⁷⁸ is 3″-OCH₃, R¹² is CHF₂ and the meaning for the combination of R¹, R², R³, R⁴, R⁹ and R¹⁰ for each individual compound corresponds in each case to one line of Table B.

Table 89i Compounds of formula I.I in which o is 1, R⁷⁸ is 3″-OCHF₂, R¹² is CHF₂ and the meaning for the combination of R¹, R², R³, R⁴, R⁹ and R¹⁰ for each individual compound corresponds in each case to one line of Table B.

Table 90i Compounds of formula I.I in which o is 1, R⁷⁸ is 3″-C₆H₅, R¹² is CHF₂ and the meaning for the combination of R¹, R², R³, R⁴, R⁹ and R¹⁰ for each individual compound corresponds in each case to one line of Table B.

Table 1j Compounds of formula I.J in which o is 0, R¹² is H and the meaning for the combination of R¹, R², R³, R⁴, R⁹ and R¹⁰ for each individual compound corresponds in each case to one line of Table B.

Table 2j Compounds of formula I.J in which o is 1, R⁷⁸ is 3″-F, R¹² is H and the meaning for the combination of R¹, R², R³, R⁴, R⁹ and R¹⁰ for each individual compound corresponds in each case to one line of Table B.

Table 3j Compounds of formula I.J in which o is 1, R⁷⁸ is 3″-C1, R¹² is H and the meaning for the combination of R¹, R², R³, R⁴, R⁹ and R¹⁰ for each individual compound corresponds in each case to one line of Table B.

Table 4j Compounds of formula I.J in which o is 1, R⁷⁸ is 3″-Br, R¹² is H and the meaning for the combination of R¹, R², R³, R⁴, R⁹ and R¹⁰ for each individual compound corresponds in each case to one line of Table B.

Table 5j Compounds of formula I.J in which o is 1, R⁷⁸ is 3″-CH₃, R¹² is H and the meaning for the combination of R¹, R², R³, R⁴, R⁹ and R¹⁰ for each individual compound corresponds in each case to one line of Table B.

Table 6j Compounds of formula I.J in which o is 1, R⁷⁸ is 3″-OCH₃, R¹² is H and the meaning for the combination of R¹, R², R³, R⁴, R⁹ and R¹⁰ for each individual compound corresponds in each case to one line of Table B.

Table 7j Compounds of formula I.J in which o is 1, R⁷⁸ is 3″-OCHF₂, R¹² is H and the meaning for the combination of R¹, R², R³, R⁴, R⁹ and R¹⁰ for each individual compound corresponds in each case to one line of Table B.

Table 8j Compounds of formula I.J in which o is 1, R⁷⁸ is 3″-C₆H₅, R¹² is H and the meaning for the combination of R1_{,} R², R³, R⁴, R⁹ and R¹⁰ for each individual compound corresponds in each case to one line of Table B.

Table 9j Compounds of formula I.J in which o is 0, R¹² is CH₃ and the meaning for the combination of R1_{,} R², R³, R⁴, R⁹ and R¹⁰ for each individual compound corresponds in each case to one line of Table B.

Table 10j Compounds of formula I.J in which o is 1, R⁷⁸ is 3″-F, R¹² is CH₃ and the meaning for the combination of R1_{,} R², R³, R⁴, R⁹ and R¹⁰ for each individual compound corresponds in each case to one line of Table B.

Table 11j Compounds of formula I.J in which o is 1, R⁷⁸ is 3″-Cl, R¹² is CH₃ and the meaning for the combination of R1_{,} R², R³, R⁴, R⁹ and R¹⁰ for each individual compound corresponds in each case to one line of Table B.

Table 12j Compounds of formula I.J in which o is 1, R⁷⁸ is 3″-Br, R¹² is CH₃ and the meaning for the combination of R¹, R², R³, R⁴, R⁹ and R¹⁰ for each individual compound corresponds in each case to one line of Table B.

Table 13j Compounds of formula I.J in which o is 1, R⁷⁸ is 3″-CH₃, R¹² is CH₃ and the meaning for the combination of R¹, R², R³, R⁴, R⁹ and R¹⁰ for each individual compound corresponds in each case to one line of Table B.

Table 14j Compounds of formula I.J in which o is 1, R⁷⁸ is 3″-OCH₃, R¹² is CH₃ and the meaning for the combination of R¹, R², R³, R⁴, R⁹ and R¹⁰ for each individual compound corresponds in each case to one line of Table B.

Table 15j Compounds of formula I.J in which o is 1, R⁷⁸ is 3″-OCHF₂, R¹² is CH₃ and the meaning for the combination of R¹, R², R³, R⁴, R⁹ and R¹⁰ for each individual compound corresponds in each case to one line of Table B.

Table 16j Compounds of formula I.J in which o is 1, R⁷⁸ is 3″-C₆H₅, R¹² is CH₃ and the meaning for the combination of R¹, R², R³, R⁴, R⁹ and R¹⁰ for each individual compound corresponds in each case to one line of Table B.

Table 17j Compounds of formula I.J in which o is 0, R¹² is CF₃ and the meaning for the combination of R¹, R², R³, R⁴, R⁹ and R¹⁰ for each individual compound corresponds in each case to one line of Table B.

Table 18j Compounds of formula I.J in which o is 1, R⁷⁸ is 3″-F, R¹² is CF₃ and the meaning for the combination of R¹, R², R³, R⁴, R⁹ and R¹⁰ for each individual compound corresponds in each case to one line of Table B.

Table 19j Compounds of formula I.J in which o is 1, R⁷⁸ is 3″-Cl, R¹² is CF₃ and the meaning for the combination of R¹, R², R³, R⁴, R⁹ and R¹⁰ for each individual compound corresponds in each case to one line of Table B.

Table 20j Compounds of formula I.J in which o is 1, R⁷⁸ is 3″-Br, R¹² is CF₃ and the meaning for the combination of R¹, R², R³, R⁴, R⁹ and R¹⁰ for each individual compound corresponds in each case to one line of Table B.

Table 21j Compounds of formula I.J in which o is 1, R⁷⁸ is 3″-CH₃, R¹² is CF₃ and the meaning for the combination of R¹, R², R³, R⁴, R⁹ and R¹⁰ for each individual compound corresponds in each case to one line of Table B.

Table 22j Compounds of formula I.J in which o is 1, R⁷⁸ is 3″-OCH₃, R¹² is CF₃ and the meaning for the combination of R¹, R², R³, R⁴, R⁹ and R¹⁰ for each individual compound corresponds in each case to one line of Table B.

Table 23j Compounds of formula I.J in which o is 1, R⁷⁸ is 3″-OCHF₂, R¹² is CF₃ and the meaning for the combination of R¹, R², R³, R⁴, R⁹ and R¹⁰ for each individual compound corresponds in each case to one line of Table B.

Table 24j Compounds of formula I.J in which o is 1, R⁷⁸ is 3″-C₆H₅, R¹² is CF₃ and the meaning for the combination of R¹, R², R³, R⁴, R⁹ and R¹⁰ for each individual compound corresponds in each case to one line of Table B.

Table 25j Compounds of formula I.J in which o is 0, R¹² is CH(CH₃)₂ and the meaning for the combination of R¹, R², R³, R⁴, R⁹ and R¹⁰ for each individual compound corresponds in each case to one line of Table B.

Table 26j Compounds of formula I.J in which o is 1, R⁷⁸ is 3″-F, R¹² is CH(CH₃)₂ and the meaning for the combination of R¹, R², R³, R⁴, R⁹ and R¹⁰ for each individual compound corresponds in each case to one line of Table B.

Table 27j Compounds of formula I.J in which o is 1, R⁷⁸ is 3″-Cl, R¹² is CH(CH₃)₂ and the meaning for the combination of R¹, R², R³, R⁴, R⁹ and R¹⁰ for each individual compound corresponds in each case to one line of Table B.

Table 28j Compounds of formula I.J in which o is 1, R⁷⁸ is 3″-Br, R¹² is CH(CH₃)₂ and the meaning for the combination of R¹, R², R³, R⁴, R⁹ and R¹⁰ for each individual compound corresponds in each case to one line of Table B.

Table 29j Compounds of formula I.J in which o is 1, R⁷⁸ is 3″-CH₃, R¹² is CH(CH₃)₂ and the meaning for the combination of R¹, R², R³, R⁴, R⁹ and R¹⁰ for each individual compound corresponds in each case to one line of Table B.

Table 30j Compounds of formula I.J in which o is 1, R⁷⁸ is 3″-OCH₃, R¹² is CH(CH₃)₂ and the meaning for the combination of R¹, R², R³, R⁴, R⁹ and R¹⁰ for each individual compound corresponds in each case to one line of Table B.

Table 31j Compounds of formula I.J in which o is 1, R⁷⁸ is 3″-OCHF₂, R¹² is CH(CH₃)₂ and the meaning for the combination of R¹, R², R³, R⁴, R⁹ and R¹⁰ for each individual compound corresponds in each case to one line of Table B.

Table 32j Compounds of formula I.J in which o is 1, R⁷⁸ is 3″-C₆H₅, R¹² is CH(CH₃)₂ and the meaning for the combination of R¹, R², R³, R⁴, R⁹ and R¹⁰ for each individual compound corresponds in each case to one line of Table B.

Table 33j Compounds of formula I.J in which o is 0, R¹² is C₂H₅ and the meaning for the combination of R¹, R², R³, R⁴, R⁹ and R¹⁰ for each individual compound corresponds in each case to one line of Table B.

Table 34j Compounds of formula I.J in which o is 1, R⁷⁸ is 3″-F, R¹² is C₂H₅ and the meaning for the combination of R¹, R², R³, R⁴, R⁹ and R¹⁰ for each individual compound corresponds in each case to one line of Table B.

Table 35j Compounds of formula I.J in which o is 1, R⁷⁸ is 3″-Cl, R¹² is C₂H₅ and the meaning for the combination of R¹, R², R³, R⁴, R⁹ and R¹⁰ for each individual compound corresponds in each case to one line of Table B.

Table 36j Compounds of formula I.J in which o is 1, R⁷⁸ is 3″-Br, R¹² is C₂H₅ and the meaning for the combination of R¹, R², R³, R⁴, R⁹ and R¹⁰ for each individual compound corresponds in each case to one line of Table B.

Table 37j Compounds of formula I.J in which o is 1, R⁷⁸ is 3″-CH₃, R¹² is C₂H₅ and the meaning for the combination of R¹, R², R³, R⁴, R⁹ and R¹⁰ for each individual compound corresponds in each case to one line of Table B.

Table 38j Compounds of formula I.J in which o is 1, R⁷⁸ is 3″-OCH₃, R¹² is C₂H₅ and the meaning for the combination of R¹, R², R³, R⁴, R⁹ and R¹⁰ for each individual compound corresponds in each case to one line of Table B.

Table 39j Compounds of formula I.J in which o is 1, R⁷⁸ is 3″-OCHF₂, R¹² is C₂H₅ and the meaning for the combination of R¹, R², R³, R⁴, R⁹ and R¹⁰ for each individual compound corresponds in each case to one line of Table B.

Table 40j Compounds of formula I.J in which o is 1, R⁷⁸ is 3″-C₆H₅, R¹² is C₂H₅ and the meaning for the combination of R¹, R², R³, R⁴, R⁹ and R¹⁰ for each individual compound corresponds in each case to one line of Table B.

Table 41j Compounds of formula I.J in which o is 0, R¹² is CHF₂ and the meaning for the combination of R¹, R², R³, R⁴, R⁹ and R¹⁰ for each individual compound corresponds in each case to one line of Table B.

Table 42j Compounds of formula I.J in which o is 1, R⁷⁸ is 3″-F, R¹² is CHF₂ and the meaning for the combination of R¹, R², R³, R⁴, R⁹ and R¹⁰ for each individual compound corresponds in each case to one line of Table B.

Table 43j Compounds of formula I.J in which o is 1, R⁷⁸ is 3″-Cl, R¹² is CHF₂ and the meaning for the combination of R¹, R², R³, R⁴, R⁹ and R¹⁰ for each individual compound corresponds in each case to one line of Table B.

Table 44j Compounds of formula I.J in which o is 1, R⁷⁸ is 3″-Br, R¹² is CHF₂ and the meaning for the combination of R¹, R², R³, R⁴, R⁹ and R¹⁰ for each individual compound corresponds in each case to one line of Table B.

Table 45j Compounds of formula I.J in which o is 1, R⁷⁸ is 3″-CH₃, R¹² is CHF₂ and the meaning for the combination of R¹, R², R³, R⁴, R⁹ and R¹⁰ for each individual compound corresponds in each case to one line of Table B.

Table 46j Compounds of formula I.J in which o is 1, R⁷⁸ is 3″-OCH₃, R¹² is CHF₂ and the meaning for the combination of R¹, R², R³, R⁴, R⁹ and R¹⁰ for each individual compound corresponds in each case to one line of Table B.

Table 47j Compounds of formula I.J in which o is 1, R⁷⁸ is 3″-OCHF₂, R¹² is CHF₂ and the meaning for the combination of R¹, R², R³, R⁴, R⁹ and R¹⁰ for each individual compound corresponds in each case to one line of Table B.

Table 48j Compounds of formula I.J in which o is 1, R⁷⁸ is 3″-C₆H₅, R¹² is CHF₂ and the meaning for the combination of R¹, R², R³, R⁴, R⁹ and R¹⁰ for each individual compound corresponds in each case to one line of Table B.

Table 1k Compounds of formula I.K in which o is 0, Y is Br and the meaning for the combination of R¹, R², R³, R⁴, R⁹ and R¹⁰ for each individual compound corresponds in each case to one line of Table B.

Table 2k Compounds of formula I.K in which o is 1, R⁷⁸ is 1″-F, Y is Br and the meaning for the combination of R¹, R², R³, R⁴, R⁹ and R¹⁰ for each individual compound corresponds in each case to one line of Table B.

Table 3k Compounds of formula I.K in which o is 1, R⁷⁸ is 1″-Cl, Y is Br and the meaning for the combination of R¹, R², R³, R⁴, R⁹ and R¹⁰ for each individual compound corresponds in each case to one line of Table B.

Table 4k Compounds of formula I.K in which o is 1, R⁷⁸ is 1″-Br, Y is Br and the meaning for the combination of R¹, R², R³, R⁴, R⁹ and R¹⁰ for each individual compound corresponds in each case to one line of Table B.

Table 5k Compounds of formula I.K in which o is 1, R⁷⁸ is 1″-CH₃, Y is Br and the meaning for the combination of R¹, R², R³, R⁴, R⁹ and R¹⁰ for each individual compound corresponds in each case to one line of Table B.

Table 6k Compounds of formula I.K in which o is 1, R⁷⁸ is 1″-OCH₃, Y is Br and the meaning for the combination of R¹, R², R³, R⁴, R⁹ and R¹⁰ for each individual compound corresponds in each case to one line of Table B.

Table 7k Compounds of formula I.K in which o is 1, R⁷⁸ is 1″-OCHF₂, Y is Br and the meaning for the combination of R¹, R², R³, R⁴, R⁹ and R¹⁰ for each individual compound corresponds in each case to one line of Table B.

Table 8k Compounds of formula I.K in which o is 1, R⁷⁸ is 1″-C₆H₅, Y is Br and the meaning for the combination of R¹, R², R³, R⁴, R⁹ and R¹⁰ for each individual compound corresponds in each case to one line of Table B.

Table 9k Compounds of formula I.K in which o is 1, R⁷⁸ is 3″-F, Y is Br and the meaning for the combination of R¹, R², R³, R⁴, R⁹ and R¹⁰ for each individual compound corresponds in each case to one line of Table B.

Table 10k Compounds of formula I.K in which o is 1, R⁷⁸ is 3″-Cl, Y is Br and the meaning for the combination of R¹, R², R³, R⁴, R⁹ and R¹⁰ for each individual compound corresponds in each case to one line of Table B.

Table 11k Compounds of formula I.K in which o is 1, R⁷⁸ is 3″-Br, Y is Br and the meaning for the combination of R¹, R², R³, R⁴, R⁹ and R¹⁰ for each individual compound corresponds in each case to one line of Table B.

Table 12k Compounds of formula I.K in which o is 1, R⁷⁸ is 3″-CH₃, Y is Br and the meaning for the combination of R¹, R², R³, R⁴, R⁹ and R¹⁰ for each individual compound corresponds in each case to one line of Table B.

Table 13k Compounds of formula I.K in which o is 1, R⁷⁸ is 3″-OCH₃, Y is Br and the meaning for the combination of R¹, R², R³, R⁴, R⁹ and R¹⁰ for each individual compound corresponds in each case to one line of Table B.

Table 14k Compounds of formula I.K in which o is 1, R⁷⁸ is 3″-OCHF₂, Y is Br and the meaning for the combination of R¹, R², R³, R⁴, R⁹ and R¹⁰ for each individual compound corresponds in each case to one line of Table B.

Table 15k Compounds of formula I.K in which o is 1, R⁷⁸ is 3″-C₆H₅, Y is Br and the meaning for the combination of R¹, R², R³, R⁴, R⁹ and R¹⁰ for each individual compound corresponds in each case to one line of Table B.

Table 16k Compounds of formula I.K in which o is 0, Y is CH₃ and the meaning for the combination of R¹, R², R³, R⁴, R⁹ and R¹⁰ for each individual compound corresponds in each case to one line of Table B.

Table 17k Compounds of formula I.K in which o is 1, R⁷⁸ is 1″-F, Y is CH₃ and the meaning for the combination of R¹, R², R³, R⁴, R⁹ and R¹⁰ for each individual compound corresponds in each case to one line of Table B.

Table 18k Compounds of formula I.K in which o is 1, R⁷⁸ is 1″-Cl, Y is CH₃ and the meaning for the combination of R¹, R², R³, R⁴, R⁹ and R¹⁰ for each individual compound corresponds in each case to one line of Table B.

Table 19k Compounds of formula I.K in which o is 1, R⁷⁸ is 1″-Br, Y is CH₃ and the meaning for the combination of R¹, R², R³, R⁴, R⁹ and R¹⁰ for each individual compound corresponds in each case to one line of Table B.

Table 20k Compounds of formula I.K in which o is 1, R⁷⁸ is 1″-CH₃, Y is CH₃ and the meaning for the combination of R¹, R², R³, R⁴, R⁹ and R¹⁰ for each individual compound corresponds in each case to one line of Table B.

Table 21k Compounds of formula I.K in which o is 1, R⁷⁸ is 1″-OCH₃, Y is CH₃ and the meaning for the combination of R¹, R², R³, R⁴, R⁹ and R¹⁰ for each individual compound corresponds in each case to one line of Table B.

Table 22k Compounds of formula I.K in which o is 1, R⁷⁸ is 1″-OCHF₂, Y is CH₃ and the meaning for the combination of R¹, R², R³, R⁴, R⁹ and R¹⁰ for each individual compound corresponds in each case to one line of Table B.

Table 23k Compounds of formula I.K in which o is 1, R⁷⁸ is 1″-C₆H₅, Y is CH₃ and the meaning for the combination of R¹, R², R³, R⁴, R⁹ and R¹⁰ for each individual compound corresponds in each case to one line of Table B.

Table 24k Compounds of formula I.K in which o is 1, R⁷⁸ is 3″-F, Y is CH₃ and the meaning for the combination of R¹, R², R³, R⁴, R⁹ and R¹⁰ for each individual compound corresponds in each case to one line of Table B.

Table 25k Compounds of formula I.K in which o is 1, R⁷⁸ is 3″-Cl, Y is CH₃ and the meaning for the combination of R¹, R², R³, R⁴, R⁹ and R¹⁰ for each individual compound corresponds in each case to one line of Table B.

Table 26k Compounds of formula I.K in which o is 1, R⁷⁸ is 3″-Br, Y is CH₃ and the meaning for the combination of R¹, R², R³, R⁴, R⁹ and R¹⁰ for each individual compound corresponds in each case to one line of Table B.

Table 27k Compounds of formula I.K in which o is 1, R⁷⁸ is 3″-CH₃, Y is CH₃ and the meaning for the combination of R¹, R², R³, R⁴, R⁹ and R¹⁰ for each individual compound corresponds in each case to one line of Table B.

Table 28k Compounds of formula I.K in which o is 1, R⁷⁸ is 3″-OCH₃, Y is CH₃ and the meaning for the combination of R¹, R², R³, R⁴, R⁹ and R¹⁰ for each individual compound corresponds in each case to one line of Table B.

Table 29k Compounds of formula I.K in which o is 1, R⁷⁸ is 3″-OCHF₂, Y is CH₃ and the meaning for the combination of R¹, R², R³, R⁴, R⁹ and R¹⁰ for each individual compound corresponds in each case to one line of Table B.

6Table 30k Compounds of formula I.K in which o is 1, R⁷⁸ is 3″-C₆H₅, Y is CH₃ and the meaning for the combination of R¹, R², R³, R⁴, R⁹ and R¹⁰ for each individual compound corresponds in each case to one line of Table B.

Table 31k Compounds of formula I.K in which o is 0, Y is CF₃ and the meaning for the combination of R¹, R², R³, R⁴, R⁹ and R¹⁰ for each individual compound corresponds in each case to one line of Table B.

Table 32k Compounds of formula I.K in which o is 1, R⁷⁸ is 1″-F, Y is CF₃ and the meaning for the combination of R¹, R², R³, R⁴, R⁹ and R¹⁰ for each individual compound corresponds in each case to one line of Table B.

Table 33k Compounds of formula I.K in which o is 1, R⁷⁸ is 1″-Cl, Y is CF₃ and the meaning for the combination of R¹, R², R³, R⁴, R⁹ and R¹⁰ for each individual compound corresponds in each case to one line of Table B.

Table 34k Compounds of formula I.K in which o is 1, R⁷⁸ is 1″-Br, Y is CF₃ and the meaning for the combination of R¹, R², R³, R⁴, R⁹ and R¹⁰ for each individual compound corresponds in each case to one line of Table B.

Table 35k Compounds of formula I.K in which o is 1, R⁷⁸ is 1″-CH₃, Y is CF₃ and the meaning for the combination of R¹, R², R³, R⁴, R⁹ and R¹⁰ for each individual compound corresponds in each case to one line of Table B.

Table 36k Compounds of formula I.K in which o is 1, R⁷⁸ is 1″-OCH₃, Y is CF₃ and the meaning for the combination of R¹, R², R³, R⁴, R⁹ and R¹⁰ for each individual compound corresponds in each case to one line of Table B.

Table 37k Compounds of formula I.K in which o is 1, R⁷⁸ is 1″-OCHF₂, Y is CF₃ and the meaning for the combination of R¹, R², R³, R⁴, R⁹ and R¹⁰ for each individual compound corresponds in each case to one line of Table B.

Table 38k Compounds of formula I.K in which o is 1, R⁷⁸ is 1″-C₆H₅, Y is CF₃ and the meaning for the combination of R¹, R², R³, R⁴, R⁹ and R¹⁰ for each individual compound corresponds in each case to one line of Table B.

Table 39k Compounds of formula I.K in which o is 1, R⁷⁸ is 3″-F, Y is CF₃ and the meaning for the combination of R¹, R², R³, R⁴, R⁹ and R¹⁰ for each individual compound corresponds in each case to one line of Table B.

Table 40k Compounds of formula I.K in which o is 1, R⁷⁸ is 3″-Cl, Y is CF₃ and the meaning for the combination of R¹, R², R³, R⁴, R⁹ and R¹⁰ for each individual compound corresponds in each case to one line of Table B.

Table 41k Compounds of formula I.K in which o is 1, R⁷⁸ is 3″-Br, Y is CF₃ and the meaning for the combination of R¹, R², R³, R⁴, R⁹ and R¹⁰ for each individual compound corresponds in each case to one line of Table B.

Table 42k Compounds of formula I.K in which o is 1, R⁷⁸ is 3″-CH₃, Y is CF₃ and the meaning for the combination of R¹, R², R³, R⁴, R⁹ and R¹⁰ for each individual compound corresponds in each case to one line of Table B.

Table 43k Compounds of formula I.K in which o is 1, R⁷⁸ is 3″-OCH₃, Y is CF₃ and the meaning for the combination of R¹, R², R³, R⁴, R⁹ and R¹⁰ for each individual compound corresponds in each case to one line of Table B.

Table 44k Compounds of formula I.K in which o is 1, R⁷⁸ is 3″-OCHF₂, Y is CF₃ and the meaning for the combination of R¹, R², R³, R⁴, R⁹ and R¹⁰ for each individual compound corresponds in each case to one line of Table B.

Table 45k Compounds of formula I.K in which o is 1, R⁷⁸ is 3″-C₆H₅, Y is CF₃ and the meaning for the combination of R¹, R², R³, R⁴, R⁹ and R¹⁰ for each individual compound corresponds in each case to one line of Table B.

Table 46k Compounds of formula I.K in which o is 0, Y is CH(CH₃)₂ and the meaning for the combination of R¹, R², R³, R⁴, R⁹ and R¹⁰ for each individual compound corresponds in each case to one line of Table B.

Table 47k Compounds of formula I.K in which o is 1, R⁷⁸ is 1″-F, Y is CH(CH₃)₂ and the meaning for the combination of R¹, R², R³, R⁴, R⁹ and R¹⁰ for each individual compound corresponds in each case to one line of Table B.

Table 48k Compounds of formula I.K in which o is 1, R⁷⁸ is 1″-Cl, Y is CH(CH₃)₂ and the meaning for the combination of R¹, R², R³, R⁴, R⁹ and R¹⁰ for each individual compound corresponds in each case to one line of Table B.

Table 49k Compounds of formula I.K in which o is 1, R⁷⁸ is 1″-Br, Y is CH(CH₃)₂ and the meaning for the combination of R¹, R², R³, R⁴, R⁹ and R¹⁰ for each individual compound corresponds in each case to one line of Table B.

Table 50k Compounds of formula I.K in which o is 1, R⁷⁸ is 1″-CH₃, Y is CH(CH₃)₂ and the meaning for the combination of R¹, R², R³, R⁴, R⁹ and R¹⁰ for each individual compound corresponds in each case to one line of Table B.

Table 51k Compounds of formula I.K in which o is 1, R⁷⁸ is 1″-OCH₃, Y is CH(CH₃)₂ and the meaning for the combination of R¹, R², R³, R⁴, R⁹ and R¹⁰ for each individual compound corresponds in each case to one line of Table B.

Table 52k Compounds of formula I.K in which o is 1, R⁷⁸ is 1″-OCHF₂, Y is CH(CH₃)₂ and the meaning for the combination of R¹, R², R³, R⁴, R⁹ and R¹⁰ for each individual compound corresponds in each case to one line of Table B.

Table 53k Compounds of formula I.K in which o is 1, R⁷⁸ is 1″-C₆H₅, Y is CH(CH₃)₂ and the meaning for the combination of R¹, R², R³, R⁴, R⁹ and R¹⁰ for each individual compound corresponds in each case to one line of Table B.

Table 54k Compounds of formula I.K in which o is 1, R⁷⁸ is 3″-F, Y is CH(CH₃)₂ and the meaning for the combination of R¹, R², R³, R⁴, R⁹ and R¹⁰ for each individual compound corresponds in each case to one line of Table B.

Table 55k Compounds of formula I.K in which o is 1, R⁷⁸ is 3″-Cl, Y is CH(CH₃)₂ and the meaning for the combination of R¹, R², R³, R⁴, R⁹ and R¹⁰ for each individual compound corresponds in each case to one line of Table B.

Table 56k Compounds of formula I.K in which o is 1, R⁷⁸ is 3″-Br, Y is CH(CH₃)₂ and the meaning for the combination of R¹, R², R³, R⁴, R⁹ and R¹⁰ for each individual compound corresponds in each case to one line of Table B.

Table 57k Compounds of formula I.K in which o is 1, R⁷⁸ is 3″-CH₃, Y is CH(CH₃)₂ and the meaning for the combination of R¹, R², R³, R⁴, R⁹ and R¹⁰ for each individual compound corresponds in each case to one line of Table B.

Table 58k Compounds of formula I.K in which o is 1, R⁷⁸ is 3″-OCH₃, Y is CH(CH₃)₂ and the meaning for the combination of R¹, R², R³, R⁴, R⁹ and R¹⁰ for each individual compound corresponds in each case to one line of Table B.

Table 59k Compounds of formula I.K in which o is 1, R⁷⁸ is 3″-OCHF₂, Y is CH(CH₃)₂ and the meaning for the combination of R¹, R², R³, R⁴, R⁹ and R¹⁰ for each individual compound corresponds in each case to one line of Table B.

Table 60k Compounds of formula I.K in which o is 1, R⁷⁸ is 3″-C₆H₅, Y is CH(CH₃)₂ and the meaning for the combination of R¹, R², R³, R⁴, R⁹ and R¹⁰ for each individual compound corresponds in each case to one line of Table B.

Table 61k Compounds of formula I.K in which o is 0, Y is C₂H₅ and the meaning for the combination of R¹, R², R³, R⁴, R⁹ and R¹⁰ for each individual compound corresponds in each case to one line of Table B.

Table 62k Compounds of formula I.K in which o is 1, R⁷⁸ is 1″-F, Y is C₂H₅ and the meaning for the combination of R¹, R², R³, R⁴, R⁹ and R¹⁰ for each individual compound corresponds in each case to one line of Table B.

Table 63k Compounds of formula I.K in which o is 1, R⁷⁸ is 1″-Cl, Y is C₂H₅ and the meaning for the combination of R¹, R², R³, R⁴, R⁹ and R¹⁰ for each individual compound corresponds in each case to one line of Table B.

Table 64k Compounds of formula I.K in which o is 1, R⁷⁸ is 1″-Br, Y is C₂H₅ and the meaning for the combination of R¹, R², R³, R⁴, R⁹ and R¹⁰ for each individual compound corresponds in each case to one line of Table B.

Table 65k Compounds of formula I.K in which o is 1, R⁷⁸ is 1″-CH₃, Y is C₂H₅ and the meaning for the combination of R¹, R², R³, R⁴, R⁹ and R¹⁰ for each individual compound corresponds in each case to one line of Table B.

Table 66k Compounds of formula I.K in which o is 1, R⁷⁸ is 1″-OCH₃, Y is C₂H₅ and the meaning for the combination of R¹, R², R³, R⁴, R⁹ and R¹⁰ for each individual compound corresponds in each case to one line of Table B.

Table 67k Compounds of formula I.K in which o is 1, R⁷⁸ is 1″-OCHF₂, Y is C₂H₅ and the meaning for the combination of R¹, R², R³, R⁴, R⁹ and R¹⁰ for each individual compound corresponds in each case to one line of Table B.

Table 68k Compounds of formula I.K in which o is 1, R⁷⁸ is 1″-C₆H₅, Y is C₂H₅ and the meaning for the combination of R¹, R², R³, R⁴, R⁹ and R¹⁰ for each individual compound corresponds in each case to one line of Table B.

Table 69k Compounds of formula I.K in which o is 1, R⁷⁸ is 3″-F, Y is C₂H₅ and the meaning for the combination of R¹, R², R³, R⁴, R⁹ and R¹⁰ for each individual compound corresponds in each case to one line of Table B.

Table 70k Compounds of formula I.K in which o is 1, R⁷⁸ is 3″-Cl, Y is C₂H₅ and the meaning for the combination of R¹, R², R³, R⁴, R⁹ and R¹⁰ for each individual compound corresponds in each case to one line of Table B.

Table 71k Compounds of formula I.K in which o is 1, R⁷⁸ is 3″-Br, Y is C₂H₅ and the meaning for the combination of R¹, R², R³, R⁴, R⁹ and R¹⁰ for each individual compound corresponds in each case to one line of Table B.

Table 72k Compounds of formula I.K in which o is 1, R⁷⁸ is 3″-CH₃, Y is C₂H₅ and the meaning for the combination of R¹, R², R³, R⁴, R⁹ and R¹⁰ for each individual compound corresponds in each case to one line of Table B.

Table 73k Compounds of formula I.K in which o is 1, R⁷⁸ is 3″-OCH₃, Y is C₂H₅ and the meaning for the combination of R¹, R², R³, R⁴, R⁹ and R¹⁰ for each individual compound corresponds in each case to one line of Table B.

Table 74k Compounds of formula I.K in which o is 1, R⁷⁸ is 3″-OCHF₂, Y is C₂H₅ and the meaning for the combination of R¹, R², R³, R⁴, R⁹ and R¹⁰ for each individual compound corresponds in each case to one line of Table B.

Table 75k Compounds of formula I.K in which o is 1, R⁷⁸ is 3″-C₆H₅, Y is C₂H₅ and the meaning for the combination of R¹, R², R³, R⁴, R⁹ and R¹⁰ for each individual compound corresponds in each case to one line of Table B.

Table 76k Compounds of formula I.K in which o is 0, Y is CHF₂ and the meaning for the combination of R¹, R², R³, R⁴, R⁹ and R¹⁰ for each individual compound corresponds in each case to one line of Table B.

Table 77k Compounds of formula I.K in which o is 1, R⁷⁸ is 1″-F, Y is CHF₂ and the meaning for the combination of R¹, R², R³, R⁴, R⁹ and R¹⁰ for each individual compound corresponds in each case to one line of Table B.

Table 78k Compounds of formula I.K in which o is 1, R⁷⁸ is 1″-Cl, Y is CHF₂ and the meaning for the combination of R¹, R², R³, R⁴, R⁹ and R¹⁰ for each individual compound corresponds in each case to one line of Table B.

Table 79k Compounds of formula I.K in which o is 1, R⁷⁸ is 1″-Br, Y is CHF₂ and the meaning for the combination of R¹, R², R³, R⁴, R⁹ and R¹⁰ for each individual compound corresponds in each case to one line of Table B.

Table 80k Compounds of formula I.K in which o is 1, R⁷⁸ is 1″-CH₃, Y is CHF₂ and the meaning for the combination of R¹, R², R³, R⁴, R⁹ and R¹⁰ for each individual compound corresponds in each case to one line of Table B.

Table 81k Compounds of formula I.K in which o is 1, R⁷⁸ is 1″-OCH₃, Y is CHF₂ and the meaning for the combination of R¹, R², R³, R⁴, R⁹ and R¹⁰ for each individual compound corresponds in each case to one line of Table B.

Table 82k Compounds of formula I.K in which o is 1, R⁷⁸ is 1″-OCHF₂, Y is CHF₂ and the meaning for the combination of R¹, R², R³, R⁴, R⁹ and R¹⁰ for each individual compound corresponds in each case to one line of Table B.

Table 83k Compounds of formula I.K in which o is 1, R⁷⁸ is 1″-C₆H₅, Y is CHF₂ and the meaning for the combination of R¹, R², R³, R⁴, R⁹ and R¹⁰ for each individual compound corresponds in each case to one line of Table B.

Table 84k Compounds of formula I.K in which o is 1, R⁷⁸ is 3″-F, Y is CHF₂ and the meaning for the combination of R¹, R², R³, R⁴, R⁹ and R¹⁰ for each individual compound corresponds in each case to one line of Table B.

Table 85k Compounds of formula I.K in which o is 1, R⁷⁸ is 3″-Cl, Y is CHF₂ and the meaning for the combination of R¹, R², R³, R⁴, R⁹ and R¹⁰ for each individual compound corresponds in each case to one line of Table B.

Table 86k Compounds of formula I.K in which o is 1, R⁷⁸ is 3″-Br, Y is CHF₂ and the meaning for the combination of R¹, R², R³, R⁴, R⁹ and R¹⁰ for each individual compound corresponds in each case to one line of Table B.

Table 87k Compounds of formula I.K in which o is 1, R⁷⁸ is 3″-CH₃, Y is CHF₂ and the meaning for the combination of R¹, R², R³, R⁴, R⁹ and R¹⁰ for each individual compound corresponds in each case to one line of Table B.

Table 88k Compounds of formula I.K in which o is 1, R⁷⁸ is 3″-OCH₃, Y is CHF₂ and the meaning for the combination of R¹, R², R³, R⁴, R⁹ and R¹⁰ for each individual compound corresponds in each case to one line of Table B.

Table 89k Compounds of formula I.K in which o is 1, R⁷⁸ is 3″-OCHF₂, Y is CHF₂ and the meaning for the combination of R¹, R², R³, R⁴, R⁹ and R¹⁰ for each individual compound corresponds in each case to one line of Table B.

Table 90k Compounds of formula I.K in which o is 1, R⁷⁸ is 3″-C₆H₅, Y is CHF₂ and the meaning for the combination of R¹, R², R³, R⁴, R⁹ and R¹⁰ for each individual compound corresponds in each case to one line of Table B.

Table 91k Compounds of formula I.K in which o is 0, Y is CN and the meaning for the combination of R¹, R², R³, R⁴, R⁹ and R¹⁰ for each individual compound corresponds in each case to one line of Table B.

Table 92k Compounds of formula I.K in which o is 1, R⁷⁸ is 1″-F, Y is CN and the meaning for the combination of R¹, R², R³, R⁴, R⁹ and R¹⁰ for each individual compound corresponds in each case to one line of Table B.

Table 93k Compounds of formula I.K in which o is 1, R⁷⁸ is 1″-Cl, Y is CN and the meaning for the combination of R¹, R², R³, R⁴, R⁹ and R¹⁰ for each individual compound corresponds in each case to one line of Table B.

Table 94k Compounds of formula I.K in which o is 1, R⁷⁸ is 1″-Br, Y is CN and the meaning for the combination of R¹, R², R³, R⁴, R⁹ and R¹⁰ for each individual compound corresponds in each case to one line of Table B.

Table 95k Compounds of formula I.K in which o is 1, R⁷⁸ is 1″-CH₃, Y is CN and the meaning for the combination of R¹, R², R³, R⁴, R⁹ and R¹⁰ for each individual compound corresponds in each case to one line of Table B.

Table 96k Compounds of formula I.K in which o is 1, R⁷⁸ is 1″-OCH₃, Y is CN and the meaning for the combination of R¹, R², R³, R⁴, R⁹ and R¹⁰ for each individual compound corresponds in each case to one line of Table B.

Table 97k Compounds of formula I.K in which o is 1, R⁷⁸ is 1″-OCHF₂, Y is CN and the meaning for the combination of R¹, R², R³, R⁴, R⁹ and R¹⁰ for each individual compound corresponds in each case to one line of Table B.

Table 98k Compounds of formula I.K in which o is 1, R⁷⁸ is 1″-C₆H₅, Y is CN and the meaning for the combination of R¹, R², R³, R⁴, R⁹ and R¹⁰ for each individual compound corresponds in each case to one line of Table B.

Table 99k Compounds of formula I.K in which o is 1, R⁷⁸ is 3″-F, Y is CN and the meaning for the combination of R¹, R², R³, R⁴, R⁹ and R¹⁰ for each individual compound corresponds in each case to one line of Table B.

Table 100k Compounds of formula I.K in which o is 1, R⁷⁸ is 3″-Cl, Y is CN and the meaning for the combination of R¹, R², R³, R⁴, R⁹ and R¹⁰ for each individual compound corresponds in each case to one line of Table B.

Table 101k Compounds of formula I.K in which o is 1, R⁷⁸ is 3″-Br, Y is CN and the meaning for the combination of R¹, R², R³, R⁴, R⁹ and R¹⁰ for each individual compound corresponds in each case to one line of Table B.

Table 102k Compounds of formula I.K in which o is 1, R⁷⁸ is 3″-CH₃, Y is CN and the meaning for the combination of R¹, R², R³, R⁴, R⁹ and R¹⁰ for each individual compound corresponds in each case to one line of Table B.

Table 103k Compounds of formula I.K in which o is 1, R⁷⁸ is 3″-OCH₃, Y is CN and the meaning for the combination of R¹, R², R³, R⁴, R⁹ and R¹⁰ for each individual compound corresponds in each case to one line of Table B.

Table 104k Compounds of formula I.K in which o is 1, R⁷⁸ is 3″-OCHF₂, Y is CN and the meaning for the combination of R¹, R², R³, R⁴, R⁹ and R¹⁰ for each individual compound corresponds in each case to one line of Table B.

Table 105k Compounds of formula I.K in which o is 1, R⁷⁸ is 3″-C₆H₅, Y is CN and the meaning for the combination of R¹, R², R³, R⁴, R⁹ and R¹⁰ for each individual compound corresponds in each case to one line of Table B.

Table 106k Compounds of formula I.K in which o is 0, Y is OCHF₂ and the meaning for the combination of R¹, R², R³, R⁴, R⁹ and R¹⁰ for each individual compound corresponds in each case to one line of Table B.

Table 107k Compounds of formula I.K in which o is 1, R⁷⁸ is 1″-F, Y is OCHF₂ and the meaning for the combination of R¹, R², R³, R⁴, R⁹ and R¹⁰ for each individual compound corresponds in each case to one line of Table B.

Table 108k Compounds of formula I.K in which o is 1, R⁷⁸ is 1″-Cl, Y is OCHF₂ and the meaning for the combination of R¹, R², R³, R⁴, R⁹ and R¹⁰ for each individual compound corresponds in each case to one line of Table B.

Table 109k Compounds of formula I.K in which o is 1, R⁷⁸ is 1″-Br, Y is OCHF₂ and the meaning for the combination of R¹, R², R³, R⁴, R⁹ and R¹⁰ for each individual compound corresponds in each case to one line of Table B.

Table 110k Compounds of formula I.K in which o is 1, R⁷⁸ is 1″-CH₃, Y is OCHF₂ and the meaning for the combination of R¹, R², R³, R⁴, R⁹ and R¹⁰ for each individual compound corresponds in each case to one line of Table B.

Table 111k Compounds of formula I.K in which o is 1, R⁷⁸ is 1″-OCH₃, Y is OCHF₂ and the meaning for the combination of R¹, R², R³, R⁴, R⁹ and R¹⁰ for each individual compound corresponds in each case to one line of Table B.

Table 112k Compounds of formula I.K in which o is 1, R⁷⁸ is 1″-OCHF₂, Y is OCHF₂ and the meaning for the combination of R¹, R², R³, R⁴, R⁹ and R¹⁰ for each individual compound corresponds in each case to one line of Table B.

Table 113k Compounds of formula I.K in which o is 1, R⁷⁸ is 1″-C₆H₅, Y is OCHF₂ and the meaning for the combination of R¹, R², R³, R⁴, R⁹ and R¹⁰ for each individual compound corresponds in each case to one line of Table B.

Table 114k Compounds of formula I.K in which o is 1, R⁷⁸ is 3″-F, Y is OCHF₂ and the meaning for the combination of R¹, R², R³, R⁴, R⁹ and R¹⁰ for each individual compound corresponds in each case to one line of Table B.

Table 115k Compounds of formula I.K in which o is 1, R⁷⁸ is 3″-Cl, Y is OCHF₂ and the meaning for the combination of R¹, R², R³, R⁴, R⁹ and R¹⁰ for each individual compound corresponds in each case to one line of Table B.

Table 116k Compounds of formula I.K in which o is 1, R⁷⁸ is 3″-Br, Y is OCHF₂ and the meaning for the combination of R¹, R², R³, R⁴, R⁹ and R¹⁰ for each individual compound corresponds in each case to one line of Table B.

Table 117k Compounds of formula I.K in which o is 1, R⁷⁸ is 3″-CH₃, Y is OCHF₂ and the meaning for the combination of R¹, R², R³, R⁴, R⁹ and R¹⁰ for each individual compound corresponds in each case to one line of Table B.

Table 118k Compounds of formula I.K in which o is 1, R⁷⁸ is 3″-OCH₃, Y is OCHF₂ and the meaning for the combination of R¹, R², R³, R⁴, R⁹ and R¹⁰ for each individual compound corresponds in each case to one line of Table B.

Table 119k Compounds of formula I.K in which o is 1, R⁷⁸ is 3″-OCHF₂, Y is OCHF₂ and the meaning for the combination of R¹, R², R³, R⁴, R⁹ and R¹⁰ for each individual compound corresponds in each case to one line of Table B.

Table 120k Compounds of formula I.K in which o is 1, R⁷⁸ is 3″-C₆H₅, Y is OCHF₂ and the meaning for the combination of R¹, R², R³, R⁴, R⁹ and R¹⁰ for each individual compound corresponds in each case to one line of Table B.

Table 121k Compounds of formula I.K in which o is 0, Y is Cl and the meaning for the combination of R¹, R², R³, R⁴, R⁹ and R¹⁰ for each individual compound corresponds in each case to one line of Table B.

Table 122k Compounds of formula I.K in which o is 1, R⁷⁸ is 1″-F, Y is Cl and the meaning for the combination of R¹, R², R³, R⁴, R⁹ and R¹⁰ for each individual compound corresponds in each case to one line of Table B.

Table 123k Compounds of formula I.K in which o is 1, R⁷⁸ is 1″-Cl, Y is Cl and the meaning for the combination of R¹, R², R³, R⁴, R⁹ and R¹⁰ for each individual compound corresponds in each case to one line of Table B.

Table 124k Compounds of formula I.K in which o is 1, R⁷⁸ is 1″-Br, Y is Cl and the meaning for the combination of R¹, R², R³, R⁴, R⁹ and R¹⁰ for each individual compound corresponds in each case to one line of Table B.

Table 125k Compounds of formula I.K in which o is 1, R⁷⁸ is 1″-CH₃, Y is Cl and the meaning for the combination of R¹, R², R³, R⁴, R⁹ and R¹⁰ for each individual compound corresponds in each case to one line of Table B.

Table 126k Compounds of formula I.K in which o is 1, R⁷⁸ is 1″-OCH₃, Y is Cl and the meaning for the combination of R¹, R², R³, R⁴, R⁹ and R¹⁰ for each individual compound corresponds in each case to one line of Table B.

Table 127k Compounds of formula I.K in which o is 1, R⁷⁸ is 1″-OCHF₂, Y is Cl and the meaning for the combination of R¹, R², R³, R⁴, R⁹ and R¹⁰ for each individual compound corresponds in each case to one line of Table B.

Table 128k Compounds of formula I.K in which o is 1, R⁷⁸ is 1″-C₆H₅, Y is Cl and the meaning for the combination of R¹, R², R³, R⁴, R⁹ and R¹⁰ for each individual compound corresponds in each case to one line of Table B.

Table 129k Compounds of formula I.K in which o is 1, R⁷⁸ is 3″-F, Y is Cl and the meaning for the combination of R¹, R², R³, R⁴, R⁹ and R¹⁰ for each individual compound corresponds in each case to one line of Table B.

Table 130k Compounds of formula I.K in which o is 1, R⁷⁸ is 3″-Cl, Y is Cl and the meaning for the combination of R¹, R², R³, R⁴, R⁹ and R¹⁰ for each individual compound corresponds in each case to one line of Table B.

Table 131k Compounds of formula I.K in which o is 1, R⁷⁸ is 3″-Br, Y is Cl and the meaning for the combination of R¹, R², R³, R⁴, R⁹ and R¹⁰ for each individual compound corresponds in each case to one line of Table B.

Table 132k Compounds of formula I.K in which o is 1, R⁷⁸ is 3″-CH₃, Y is Cl and the meaning for the combination of R¹, R², R³, R⁴, R⁹ and R¹⁰ for each individual compound corresponds in each case to one line of Table B.

Table 133k Compounds of formula I.K in which o is 1, R⁷⁸ is 3″-OCH₃, Y is Cl and the meaning for the combination of R¹, R², R³, R⁴, R⁹ and R¹⁰ for each individual compound corresponds in each case to one line of Table B.

Table 134k Compounds of formula I.K in which o is 1, R⁷⁸ is 3″-OCHF₂, Y is Cl and the meaning for the combination of R¹, R², R³, R⁴, R⁹ and R¹⁰ for each individual compound corresponds in each case to one line of Table B.

Table 135k Compounds of formula I.K in which o is 1, R⁷⁸ is 3″-C₆H₅, Y is Cl and the meaning for the combination of R¹, R², R³, R⁴, R⁹ and R¹⁰ for each individual compound corresponds in each case to one line of Table B.

Table 136k Compounds of formula I.K in which o is 0, Y is F and the meaning for the combination of R¹, R², R³, R⁴, R⁹ and R¹⁰ for each individual compound corresponds in each case to one line of Table B.

Table 137k Compounds of formula I.K in which o is 1, R⁷⁸ is 1″-F, Y is F and the meaning for the combination of R¹, R², R³, R⁴, R⁹ and R¹⁰ for each individual compound corresponds in each case to one line of Table B.

Table 138k Compounds of formula I.K in which o is 1, R⁷⁸ is 1″-Cl, Y is F and the meaning for the combination of R¹, R², R³, R⁴, R⁹ and R¹⁰ for each individual compound corresponds in each case to one line of Table B.

Table 139k Compounds of formula I.K in which o is 1, R⁷⁸ is 1″-Br, Y is F and the meaning for the combination of R¹, R², R³, R⁴, R⁹ and R¹⁰ for each individual compound corresponds in each case to one line of Table B.

Table 140k Compounds of formula I.K in which o is 1, R⁷⁸ is 1″-CH₃, Y is F and the meaning for the combination of R¹, R², R³, R⁴, R⁹ and R¹⁰ for each individual compound corresponds in each case to one line of Table B.

Table 141k Compounds of formula I.K in which o is 1, R⁷⁸ is 1″-OCH₃, Y is F and the meaning for the combination of R¹, R², R³, R⁴, R⁹ and R¹⁰ for each individual compound corresponds in each case to one line of Table B.

Table 142k Compounds of formula I.K in which o is 1, R⁷⁸ is 1″-OCHF₂, Y is F and the meaning for the combination of R¹, R², R³, R⁴, R⁹ and R¹⁰ for each individual compound corresponds in each case to one line of Table B.

Table 143k Compounds of formula I.K in which o is 1, R⁷⁸ is 1″-C₆H₅, Y is F and the meaning for the combination of R¹, R², R³, R⁴, R⁹ and R¹⁰ for each individual compound corresponds in each case to one line of Table B.

Table 144k Compounds of formula I.K in which o is 1, R⁷⁸ is 3″-F, Y is F and the meaning for the combination of R¹, R², R³, R⁴, R⁹ and R¹⁰ for each individual compound corresponds in each case to one line of Table B.

Table 145k Compounds of formula I.K in which o is 1, R⁷⁸ is 3″-Cl, Y is F and the meaning for the combination of R¹, R², R³, R⁴, R⁹ and R¹⁰ for each individual compound corresponds in each case to one line of Table B.

Table 146k Compounds of formula I.K in which o is 1, R⁷⁸ is 3″-Br, Y is F and the meaning for the combination of R¹, R², R³, R⁴, R⁹ and R¹⁰ for each individual compound corresponds in each case to one line of Table B.

Table 147k Compounds of formula I.K in which o is 1, R⁷⁸ is 3″-CH₃, Y is F and the meaning for the combination of R¹, R², R³, R⁴, R⁹ and R¹⁰ for each individual compound corresponds in each case to one line of Table B.

Table 148k Compounds of formula I.K in which o is 1, R⁷⁸ is 3″-OCH₃, Y is F and the meaning for the combination of R¹, R², R³, R⁴, R⁹ and R¹⁰ for each individual compound corresponds in each case to one line of Table B.

Table 149k Compounds of formula I.K in which o is 1, R⁷⁸ is 3″-OCHF₂, Y is F and the meaning for the combination of R¹, R², R³, R⁴, R⁹ and R¹⁰ for each individual compound corresponds in each case to one line of Table B.

Table 150k Compounds of formula I.K in which o is 1, R⁷⁸ is 3″-C₆H₅, Y is F and the meaning for the combination of R¹, R², R³, R⁴, R⁹ and R¹⁰ for each individual compound corresponds in each case to one line of Table B.

Table 1 ka Compounds of formula I.Ka in which o is 0, Y is Br and the meaning for the combination of R¹, R², R³, R⁴, R⁹ and R¹⁰ for each individual compound corresponds in each case to one line of Table B.

Table 2ka Compounds of formula I.Ka in which o is 1, R⁷⁸ is 1″-F, Y is Br and the meaning for the combination of R¹, R², R³, R⁴, R⁹ and R¹⁰ for each individual compound corresponds in each case to one line of Table B.

Table 3ka Compounds of formula I.Ka in which o is 1, R⁷⁸ is 1″-Cl, Y is Br and the meaning for the combination of R¹, R², R³, R⁴, R⁹ and R¹⁰ for each individual compound corresponds in each case to one line of Table B.

Table 4ka Compounds of formula I.Ka in which o is 1, R⁷⁸ is 1″-Br, Y is Br and the meaning for the combination of R¹, R², R³, R⁴, R⁹ and R¹⁰ for each individual compound corresponds in each case to one line of Table B.

Table 5ka Compounds of formula I.Ka in which o is 1, R⁷⁸ is 1″-CH₃, Y is Br and the meaning for the combination of R¹, R², R³, R⁴, R⁹ and R¹⁰ for each individual compound corresponds in each case to one line of Table B.

Table 6ka Compounds of formula I.Ka in which o is 1, R⁷⁸ is 1″-OCH₃, Y is Br and the meaning for the combination of R¹, R², R³, R⁴, R⁹ and R¹⁰ for each individual compound corresponds in each case to one line of Table B.

Table 7ka Compounds of formula I.Ka in which o is 1, R⁷⁸ is 1″-OCHF₂, Y is Br and the meaning for the combination of R¹, R², R³, R⁴, R⁹ and R¹⁰ for each individual compound corresponds in each case to one line of Table B.

Table 8ka Compounds of formula I.Ka in which o is 1, R⁷⁸ is 1″-C₆H₅, Y is Br and the meaning for the combination of R¹, R², R³, R⁴, R⁹ and R¹⁰ for each individual compound corresponds in each case to one line of Table B.

Table 9ka Compounds of formula I.Ka in which o is 1, R⁷⁸ is 2″-F, Y is Br and the meaning for the combination of R¹, R², R³, R⁴, R⁹ and R¹⁰ for each individual compound corresponds in each case to one line of Table B.

Table 10ka Compounds of formula I.Ka in which o is 1, R⁷⁸ is 2″-Cl, Y is Br and the meaning for the combination of R¹, R², R³, R⁴, R⁹ and R¹⁰ for each individual compound corresponds in each case to one line of Table B.

Table 11ka Compounds of formula I.Ka in which o is 1, R⁷⁸ is 2″-Br, Y is Br and the meaning for the combination of R¹, R², R³, R⁴, R⁹ and R¹⁰ for each individual compound corresponds in each case to one line of Table B.

Table 12ka Compounds of formula I.Ka in which o is 1, R⁷⁸ is 2″-CH₃, Y is Br and the meaning for the combination of R¹, R², R³, R⁴, R⁹ and R¹⁰ for each individual compound corresponds in each case to one line of Table B.

Table 13ka Compounds of formula I.Ka in which o is 1, R⁷⁸ is 2″-OCH₃, Y is Br and the meaning for the combination of R¹, R², R³, R⁴, R⁹ and R¹⁰ for each individual compound corresponds in each case to one line of Table B.

Table 14ka Compounds of formula I.Ka in which o is 1, R⁷⁸ is 2″-OCHF₂, Y is Br and the meaning for the combination of R¹, R², R³, R⁴, R⁹ and R¹⁰ for each individual compound corresponds in each case to one line of Table B.

Table 15ka Compounds of formula I.Ka in which o is 1, R⁷⁸ is 2″-C₆H₅, Y is Br and the meaning for the combination of R¹, R², R³, R⁴, R⁹ and R¹⁰ for each individual compound corresponds in each case to one line of Table B.

Table 16ka Compounds of formula I.Ka in which o is 1, R⁷⁸ is 3″-F, Y is Br and the meaning for the combination of R¹, R², R³, R⁴, R⁹ and R¹⁰ for each individual compound corresponds in each case to one line of Table B.

Table 17ka Compounds of formula I.Ka in which o is 1, R⁷⁸ is 3″-Cl, Y is Br and the meaning for the combination of R¹, R², R³, R⁴, R⁹ and R¹⁰ for each individual compound corresponds in each case to one line of Table B.

Table 18ka Compounds of formula I.Ka in which o is 1, R⁷⁸ is 3″-Br, Y is Br and the meaning for the combination of R¹, R², R³, R⁴, R⁹ and R¹⁰ for each individual compound corresponds in each case to one line of Table B.

Table 19ka Compounds of formula I.Ka in which o is 1, R⁷⁸ is 3″-CH₃, Y is Br and the meaning for the combination of R¹, R², R³, R⁴, R⁹ and R¹⁰ for each individual compound corresponds in each case to one line of Table B.

Table 20ka Compounds of formula I.Ka in which o is 1, R⁷⁸ is 3″-OCH₃, Y is Br and the meaning for the combination of R¹, R², R³, R⁴, R⁹ and R¹⁰ for each individual compound corresponds in each case to one line of Table B.

Table 21ka Compounds of formula I.Ka in which o is 1, R⁷⁸ is 3″-OCHF₂, Y is Br and the meaning for the combination of R¹, R², R³, R⁴, R⁹ and R¹⁰ for each individual compound corresponds in each case to one line of Table B.

Table 22ka Compounds of formula I.Ka in which o is 1, R⁷⁸ is 3″-C₆H₅, Y is Br and the meaning for the combination of R¹, R², R³, R⁴, R⁹ and R¹⁰ for each individual compound corresponds in each case to one line of Table B.

Table 23ka Compounds of formula I.Ka in which o is 1, R⁷⁸ is 3″-CHF₂, Y is Br and the meaning for the combination of R¹, R², R³, R⁴, R⁹ and R¹⁰ for each individual compound corresponds in each case to one line of Table B.

Table 24ka Compounds of formula I.Ka in which o is 1, R⁷⁸ is 3″-OCF₃, Y is Br and the meaning for the combination of R¹, R², R³, R⁴, R⁹ and R¹⁰ for each individual compound corresponds in each case to one line of Table B.

Table 25ka Compounds of formula I.Ka in which o is 1, R⁷⁸ is 3″-CF₃, Y is Br and the meaning for the combination of R¹, R², R³, R⁴, R⁹ and R¹⁰ for each individual compound corresponds in each case to one line of Table B.

Table 26ka Compounds of formula I.Ka in which o is 1, R⁷⁸ is 3″-CN Y is Br and the meaning for the combination of R¹, R², R³, R⁴, R⁹ and R¹⁰ for each individual compound corresponds in each case to one line of Table B.

Table 27ka Compounds of formula I.Ka in which o is 0, Y is CH₃ and the meaning for the combination of R¹, R², R³, R⁴, R⁹ and R¹⁰ for each individual compound corresponds in each case to one line of Table B.

Table 28ka Compounds of formula I.Ka in which o is 1, R⁷⁸ is 1″-F, Y is CH₃ and the meaning for the combination of R¹, R², R³, R⁴, R⁹ and R¹⁰ for each individual compound corresponds in each case to one line of Table B.

Table 29ka Compounds of formula I.Ka in which o is 1, R⁷⁸ is 1″-Cl, Y is CH₃ and the meaning for the combination of R¹, R², R³, R⁴, R⁹ and R¹⁰ for each individual compound corresponds in each case to one line of Table B.

Table 30ka Compounds of formula I.Ka in which o is 1, R⁷⁸ is 1″-Br, Y is CH₃ and the meaning for the combination of R¹, R², R³, R⁴, R⁹ and R¹⁰ for each individual compound corresponds in each case to one line of Table B.

Table 31ka Compounds of formula I.Ka in which o is 1, R⁷⁸ is 1″-CH₃, Y is CH₃ and the meaning for the combination of R¹, R², R³, R⁴, R⁹ and R¹⁰ for each individual compound corresponds in each case to one line of Table B.

Table 32ka Compounds of formula I.Ka in which o is 1, R⁷⁸ is 1″-OCH₃, Y is CH₃ and the meaning for the combination of R¹, R², R³, R⁴, R⁹ and R¹⁰ for each individual compound corresponds in each case to one line of Table B.

Table 33ka Compounds of formula I.Ka in which o is 1, R⁷⁸ is 1″-OCHF₂, Y is CH₃ and the meaning for the combination of R¹, R², R³, R⁴, R⁹ and R¹⁰ for each individual compound corresponds in each case to one line of Table B.

Table 34ka Compounds of formula I.Ka in which o is 1, R⁷⁸ is 1″-C₆H₅, Y is CH₃ and the meaning for the combination of R¹, R², R³, R⁴, R⁹ and R¹⁰ for each individual compound corresponds in each case to one line of Table B.

Table 35ka Compounds of formula I.Ka in which o is 1, R⁷⁸ is 2″-F, Y is CH₃ and the meaning for the combination of R¹, R², R³, R⁴, R⁹ and R¹⁰ for each individual compound corresponds in each case to one line of Table B.

Table 36ka Compounds of formula I.Ka in which o is 1, R⁷⁸ is 2″-Cl, Y is CH₃ and the meaning for the combination of R¹, R², R³, R⁴, R⁹ and R¹⁰ for each individual compound corresponds in each case to one line of Table B.

Table 37ka Compounds of formula I.Ka in which o is 1, R⁷⁸ is 2″-Br, Y is CH₃ and the meaning for the combination of R¹, R², R³, R⁴, R⁹ and R¹⁰ for each individual compound corresponds in each case to one line of Table B.

Table 38ka Compounds of formula I.Ka in which o is 1, R⁷⁸ is 2″-CH₃, Y is CH₃ and the meaning for the combination of R¹, R², R³, R⁴, R⁹ and R¹⁰ for each individual compound corresponds in each case to one line of Table B.

Table 39ka Compounds of formula I.Ka in which o is 1, R⁷⁸ is 2″-OCH₃, Y is CH₃ and the meaning for the combination of R¹, R², R³, R⁴, R⁹ and R¹⁰ for each individual compound corresponds in each case to one line of Table B.

Table 40ka Compounds of formula I.Ka in which o is 1, R⁷⁸ is 2″-OCHF₂, Y is CH₃ and the meaning for the combination of R¹, R², R³, R⁴, R⁹ and R¹⁰ for each individual compound corresponds in each case to one line of Table B.

Table 41ka Compounds of formula I.Ka in which o is 1, R⁷⁸ is 2″-C₆H₅, Y is CH₃ and the meaning for the combination of R¹, R², R³, R⁴, R⁹ and R¹⁰ for each individual compound corresponds in each case to one line of Table B.

Table 42ka Compounds of formula I.Ka in which o is 1, R⁷⁸ is 3″-F, Y is CH₃ and the meaning for the combination of R¹, R², R³, R⁴, R⁹ and R¹⁰ for each individual compound corresponds in each case to one line of Table B.

Table 43ka Compounds of formula I.Ka in which o is 1, R⁷⁸ is 3″-Cl, Y is CH₃ and the meaning for the combination of R¹, R², R³, R⁴, R⁹ and R¹⁰ for each individual compound corresponds in each case to one line of Table B.

Table 44ka Compounds of formula I.Ka in which o is 1, R⁷⁸ is 3″-Br, Y is CH₃ and the meaning for the combination of R¹, R², R³, R⁴, R⁹ and R¹⁰ for each individual compound corresponds in each case to one line of Table B.

Table 45ka Compounds of formula I.Ka in which o is 1, R⁷⁸ is 3″-CH₃, Y is CH₃ and the meaning for the combination of R¹, R², R³, R⁴, R⁹ and R¹⁰ for each individual compound corresponds in each case to one line of Table B. Table 46ka Compounds of formula I.Ka in which o is 1, R⁷⁸ is 3″-OCH₃, Y is CH₃ and the meaning for the combination of R¹, R², R³, R⁴, R⁹ and R¹⁰ for each individual compound corresponds in each case to one line of Table B.

Table 47ka Compounds of formula I.Ka in which o is 1, R⁷⁸ is 3″-OCHF₂, Y is CH₃ and the meaning for the combination of R¹, R², R³, R⁴, R⁹ and R¹⁰ for each individual compound corresponds in each case to one line of Table B.

Table 48ka Compounds of formula I.Ka in which o is 1, R⁷⁸ is 3″-C₆H₅, Y is CH₃ and the meaning for the combination of R¹, R², R³, R⁴, R⁹ and R¹⁰ for each individual compound corresponds in each case to one line of Table B.

Table 49ka Compounds of formula I.Ka in which o is 1, R⁷⁸ is 3″-CHF₂, Y is CH₃ and the meaning for the combination of R¹, R², R³, R⁴, R⁹ and R¹⁰ for each individual compound corresponds in each case to one line of Table B.

Table 50ka Compounds of formula I.Ka in which o is 1, R⁷⁸ is 3″-OCF₃, Y is CH₃ and the meaning for the combination of R¹, R², R³, R⁴, R⁹ and R¹⁰ for each individual compound corresponds in each case to one line of Table B.

Table 51ka Compounds of formula I.Ka in which o is 1, R⁷⁸ is 3″-CF₃, Y is CH₃ and the meaning for the combination of R¹, R², R³, R⁴, R⁹ and R¹⁰ for each individual compound corresponds in each case to one line of Table B.

Table 52ka Compounds of formula I.Ka in which o is 1, R⁷⁸ is 3″-CN Y is CH₃ and the meaning for the combination of R¹, R², R³, R⁴, R⁹ and R¹⁰ for each individual compound corresponds in each case to one line of Table B.

Table 53ka Compounds of formula I.Ka in which o is 0, Y is CF₃ and the meaning for the combination of R¹, R², R³, R⁴, R⁹ and R¹⁰ for each individual compound corresponds in each case to one line of Table B.

Table 54ka Compounds of formula I.Ka in which o is 1, R⁷⁸ is 1″-F, Y is CF₃ and the meaning for the combination of R¹, R², R³, R⁴, R⁹ and R¹⁰ for each individual compound corresponds in each case to one line of Table B.

Table 55ka Compounds of formula I.Ka in which o is 1, R⁷⁸ is 1″-Cl, Y is CF₃ and the meaning for the combination of R¹, R², R³, R⁴, R⁹ and R¹⁰ for each individual compound corresponds in each case to one line of Table B.

Table 56ka Compounds of formula I.Ka in which o is 1, R⁷⁸ is 1″-Br, Y is CF₃ and the meaning for the combination of R¹, R², R³, R⁴, R⁹ and R¹⁰ for each individual compound corresponds in each case to one line of Table B.

Table 57ka Compounds of formula I.Ka in which o is 1, R⁷⁸ is 1″-CH₃, Y is CF₃ and the meaning for the combination of R¹, R², R³, R⁴, R⁹ and R¹⁰ for each individual compound corresponds in each case to one line of Table B.

Table 58ka Compounds of formula I.Ka in which o is 1, R⁷⁸ is 1″-OCH₃, Y is CF₃ and the meaning for the combination of R¹, R², R³, R⁴, R⁹ and R¹⁰ for each individual compound corresponds in each case to one line of Table B.

Table 59ka Compounds of formula I.Ka in which o is 1, R⁷⁸ is 1″-OCHF₂, Y is CF₃ and the meaning for the combination of R¹, R², R³, R⁴, R⁹ and R¹⁰ for each individual compound corresponds in each case to one line of Table B.

Table 60ka Compounds of formula I.Ka in which o is 1, R⁷⁸ is 1″-C₆H₅, Y is CF₃ and the meaning for the combination of R¹, R², R³, R⁴, R⁹ and R¹⁰ for each individual compound corresponds in each case to one line of Table B.

Table 61ka Compounds of formula I.Ka in which o is 1, R⁷⁸ is 2″-F, Y is CF₃ and the meaning for the combination of R¹, R², R³, R⁴, R⁹ and R¹⁰ for each individual compound corresponds in each case to one line of Table B.

Table 62ka Compounds of formula I.Ka in which o is 1, R⁷⁸ is 2″-Cl, Y is CF₃ and the meaning for the combination of R¹, R², R³, R⁴, R⁹ and R¹⁰ for each individual compound corresponds in each case to one line of Table B.

Table 63ka Compounds of formula I.Ka in which o is 1, R⁷⁸ is 2″-Br, Y is CF₃ and the meaning for the combination of R¹, R², R³, R⁴, R⁹ and R¹⁰ for each individual compound corresponds in each case to one line of Table B.

Table 64ka Compounds of formula I.Ka in which o is 1, R⁷⁸ is 2″-CH₃, Y is CF₃ and the meaning for the combination of R¹, R², R³, R⁴, R⁹ and R¹⁰ for each individual compound corresponds in each case to one line of Table B.

Table 65ka Compounds of formula I.Ka in which o is 1, R⁷⁸ is 2″-OCH₃, Y is CF₃ and the meaning for the combination of R¹, R², R³, R⁴, R⁹ and R¹⁰ for each individual compound corresponds in each case to one line of Table B.

Table 66ka Compounds of formula I.Ka in which o is 1, R⁷⁸ is 2″-OCHF₂, Y is CF₃ and the meaning for the combination of R¹, R², R³, R⁴, R⁹ and R¹⁰ for each individual compound corresponds in each case to one line of Table B.

Table 67ka Compounds of formula I.Ka in which o is 1, R⁷⁸ is 2″-C₆H₅, Y is CF₃ and the meaning for the combination of R¹, R², R³, R⁴, R⁹ and R¹⁰ for each individual compound corresponds in each case to one line of Table B.

Table 68ka Compounds of formula I.Ka in which o is 1, R⁷⁸ is 3″-F, Y is CF₃ and the meaning for the combination of R¹, R², R³, R⁴, R⁹ and R¹⁰ for each individual compound corresponds in each case to one line of Table B.

Table 69ka Compounds of formula I.Ka in which o is 1, R⁷⁸ is 3″-Cl, Y is CF₃ and the meaning for the combination of R¹, R², R³, R⁴, R⁹ and R¹⁰ for each individual compound corresponds in each case to one line of Table B.

Table 70ka Compounds of formula I.Ka in which o is 1, R⁷⁸ is 3″-Br, Y is CF₃ and the meaning for the combination of R¹, R², R³, R⁴, R⁹ and R¹⁰ for each individual compound corresponds in each case to one line of Table B.

Table 71ka Compounds of formula I.Ka in which o is 1, R⁷⁸ is 3″-CH₃, Y is CF₃ and the meaning for the combination of R¹, R², R³, R⁴, R⁹ and R¹⁰ for each individual compound corresponds in each case to one line of Table B.

Table 72ka Compounds of formula I.Ka in which o is 1, R⁷⁸ is 3″-OCH₃, Y is CF₃ and the meaning for the combination of R¹, R², R³, R⁴, R⁹ and R¹⁰ for each individual compound corresponds in each case to one line of Table B.

Table 73ka Compounds of formula I.Ka in which o is 1, R⁷⁸ is 3″-OCHF₂, Y is CF₃ and the meaning for the combination of R¹, R², R³, R⁴, R⁹ and R¹⁰ for each individual compound corresponds in each case to one line of Table B.

Table 74ka Compounds of formula I.Ka in which o is 1, R⁷⁸ is 3″-C₆H₅, Y is CF₃ and the meaning for the combination of R¹, R², R³, R⁴, R⁹ and R¹⁰ for each individual compound corresponds in each case to one line of Table B.

Table 75ka Compounds of formula I.Ka in which o is 1, R⁷⁸ is 3″-CHF₂, Y is CF₃ and the meaning for the combination of R¹, R², R³, R⁴, R⁹ and R¹⁰ for each individual compound corresponds in each case to one line of Table B.

Table 76ka Compounds of formula I.Ka in which o is 1, R⁷⁸ is 3″-OCF₃, Y is CF₃ and the meaning for the combination of R¹, R², R³, R⁴, R⁹ and R¹⁰ for each individual compound corresponds in each case to one line of Table B.

Table 77ka Compounds of formula I.Ka in which o is 1, R⁷⁸ is 3″-CF₃, Y is CF₃ and the meaning for the combination of R¹, R², R³, R⁴, R⁹ and R¹⁰ for each individual compound corresponds in each case to one line of Table B.

Table 78ka Compounds of formula I.Ka in which o is 1, R⁷⁸ is 3″-CN Y is CF₃and the meaning for the combination of R¹, R², R³, R⁴, R⁹ and R¹⁰ for each individual compound corresponds in each case to one line of Table B.

Table 79ka Compounds of formula I.Ka in which o is 0, Y is CH(CH₃)₂ and the meaning for the combination of R¹, R², R³, R⁴, R⁹ and R¹⁰ for each individual compound corresponds in each case to one line of Table B.

Table 80ka Compounds of formula I.Ka in which o is 1, R⁷⁸ is 1″-F, Y is CH(CH₃)₂ and the meaning for the combination of R¹, R², R³, R⁴, R⁹ and R¹⁰ for each individual compound corresponds in each case to one line of Table B.

Table 81ka Compounds of formula I.Ka in which o is 1, R⁷⁸ is 1″-Cl, Y is CH(CH₃)₂ and the meaning for the combination of R¹, R², R³, R⁴, R⁹ and R¹⁰ for each individual compound corresponds in each case to one line of Table B.

Table 82ka Compounds of formula I.Ka in which o is 1, R⁷⁸ is 1″-Br, Y is CH(CH₃)₂ and the meaning for the combination of R¹, R², R³, R⁴, R⁹ and R¹⁰ for each individual compound corresponds in each case to one line of Table B.

Table 83ka Compounds of formula I.Ka in which o is 1, R⁷⁸ is 1″-CH₃, Y is CH(CH₃)₂ and the meaning for the combination of R¹, R², R³, R⁴, R⁹ and R¹⁰ for each individual compound corresponds in each case to one line of Table B.

Table 84ka Compounds of formula I.Ka in which o is 1, R⁷⁸ is 1″-OCH₃, Y is CH(CH₃)₂ and the meaning for the combination of R¹, R², R³, R⁴, R⁹ and R¹⁰ for each individual compound corresponds in each case to one line of Table B.

Table 85ka Compounds of formula I.Ka in which o is 1, R⁷⁸ is 1″-OCHF₂, Y is CH(CH₃)₂ and the meaning for the combination of R¹, R², R³, R⁴, R⁹ and R¹⁰ for each individual compound corresponds in each case to one line of Table B.

Table 86ka Compounds of formula I.Ka in which o is 1, R⁷⁸ is 1″-C₆H₅, Y is CH(CH₃)₂ and the meaning for the combination of R¹, R², R³, R⁴, R⁹ and R¹⁰ for each individual compound corresponds in each case to one line of Table B.

Table 87ka Compounds of formula I.Ka in which o is 1, R⁷⁸ is 2″-F, Y is CH(CH₃)₂ and the meaning for the combination of R¹, R², R³, R⁴, R⁹ and R¹⁰ for each individual compound corresponds in each case to one line of Table B.

Table 88ka Compounds of formula I.Ka in which o is 1, R⁷⁸ is 2″-Cl, Y is CH(CH₃)₂ and the meaning for the combination of R¹, R², R³, R⁴, R⁹ and R¹⁰ for each individual compound corresponds in each case to one line of Table B.

Table 89ka Compounds of formula I.Ka in which o is 1, R⁷⁸ is 2″-Br, Y is CH(CH₃)₂ and the meaning for the combination of R¹, R², R³, R⁴, R⁹ and R¹⁰ for each individual compound corresponds in each case to one line of Table B.

Table 90ka Compounds of formula I.Ka in which o is 1, R⁷⁸ is 2″-CH₃, Y is CH(CH₃)₂ and the meaning for the combination of R¹, R², R³, R⁴, R⁹ and R¹⁰ for each individual compound corresponds in each case to one line of Table B.

Table 91ka Compounds of formula I.Ka in which o is 1, R⁷⁸ is 2″-OCH₃, Y is CH(CH₃)₂ and the meaning for the combination of R¹, R², R³, R⁴, R⁹ and R¹⁰ for each individual compound corresponds in each case to one line of Table B.

Table 92ka Compounds of formula I.Ka in which o is 1, R⁷⁸ is 2″-OCHF₂, Y is CH(CH₃)₂ and the meaning for the combination of R¹, R², R³, R⁴, R⁹ and R¹⁰ for each individual compound corresponds in each case to one line of Table B.

Table 93ka Compounds of formula I.Ka in which o is 1, R⁷⁸ is 2″-C₆H₅, Y is CH(CH₃)₂ and the meaning for the combination of R¹, R², R³, R⁴, R⁹ and R¹⁰ for each individual compound corresponds in each case to one line of Table B.

Table 94ka Compounds of formula I.Ka in which o is 1, R⁷⁸ is 3″-F, Y is CH(CH₃)₂ and the meaning for the combination of R¹, R², R³, R⁴, R⁹ and R¹⁰ for each individual compound corresponds in each case to one line of Table B.

Table 95ka Compounds of formula I.Ka in which o is 1, R⁷⁸ is 3″-Cl, Y is CH(CH₃)₂ and the meaning for the combination of R¹, R², R³, R⁴, R⁹ and R¹⁰ for each individual compound corresponds in each case to one line of Table B.

Table 96ka Compounds of formula I.Ka in which o is 1, R⁷⁸ is 3″-Br, Y is CH(CH₃)₂ and the meaning for the combination of R¹, R², R³, R⁴, R⁹ and R¹⁰ for each individual compound corresponds in each case to one line of Table B.

Table 97ka Compounds of formula I.Ka in which o is 1, R⁷⁸ is 3″-CH₃, Y is CH(CH₃)₂ and the meaning for the combination of R¹, R², R³, R⁴, R⁹ and R¹⁰ for each individual compound corresponds in each case to one line of Table B.

Table 98ka Compounds of formula I.Ka in which o is 1, R⁷⁸ is 3″-OCH₃, Y is CH(CH₃)₂ and the meaning for the combination of R¹, R², R³, R⁴, R⁹ and R¹⁰ for each individual compound corresponds in each case to one line of Table B.

Table 99ka Compounds of formula I.Ka in which o is 1, R⁷⁸ is 3″-OCHF₂, Y is CH(CH₃)₂ and the meaning for the combination of R¹, R², R³, R⁴, R⁹ and R¹⁰ for each individual compound corresponds in each case to one line of Table B.

Table 100ka Compounds of formula I.Ka in which o is 1, R⁷⁸ is 3″-C₆H₅, Y is CH(CH₃)₂ and the meaning for the combination of R¹, R², R³, R⁴, R⁹ and R¹⁰ for each individual compound corresponds in each case to one line of Table B.

Table 101ka Compounds of formula I.Ka in which o is 0, Y is C₂H₅ and the meaning for the combination of R¹, R², R³, R⁴, R⁹ and R¹⁰ for each individual compound corresponds in each case to one line of Table B.

Table 102ka Compounds of formula I.Ka in which o is 1, R⁷⁸ is 1″-F, Y is C₂H₅ and the meaning for the combination of R¹, R², R³, R⁴, R⁹ and R¹⁰ for each individual compound corresponds in each case to one line of Table B.

Table 103ka Compounds of formula I.Ka in which o is 1, R⁷⁸ is 1″-Cl, Y is C₂H₅ and the meaning for the combination of R¹, R², R³, R⁴, R⁹ and R¹⁰ for each individual compound corresponds in each case to one line of Table B.

Table 104ka Compounds of formula I.Ka in which o is 1, R⁷⁸ is 1″-Br, Y is C₂H₅ and the meaning for the combination of R¹, R², R³, R⁴, R⁹ and R¹⁰ for each individual compound corresponds in each case to one line of Table B.

Table 105ka Compounds of formula I.Ka in which o is 1, R⁷⁸ is 1″-CH₃, Y is C₂H₅ and the meaning for the combination of R¹, R², R³, R⁴, R⁹ and R¹⁰ for each individual compound corresponds in each case to one line of Table B.

Table 106ka Compounds of formula I.Ka in which o is 1, R⁷⁸ is 1″-OCH₃, Y is C₂H₅ and the meaning for the combination of R¹, R², R³, R⁴, R⁹ and R¹⁰ for each individual compound corresponds in each case to one line of Table B.

Table 107ka Compounds of formula I.Ka in which o is 1, R⁷⁸ is 1″-OCHF₂, Y is C₂H₅ and the meaning for the combination of R¹, R², R³, R⁴, R⁹ and R¹⁰ for each individual compound corresponds in each case to one line of Table B.

Table 108ka Compounds of formula I.Ka in which o is 1, R⁷⁸ is 1″-C₆H₅, Y is C₂H₅ and the meaning for the combination of R¹, R², R³, R⁴, R⁹ and R¹⁰ for each individual compound corresponds in each case to one line of Table B.

Table 109ka Compounds of formula I.Ka in which o is 1, R⁷⁸ is 2″-F, Y is C₂H₅ and the meaning for the combination of R¹, R², R³, R⁴, R⁹ and R¹⁰ for each individual compound corresponds in each case to one line of Table B.

Table 110ka Compounds of formula I.Ka in which o is 1, R⁷⁸ is 2″-Cl, Y is C₂H₅ and the meaning for the combination of R¹, R², R³, R⁴, R⁹ and R¹⁰ for each individual compound corresponds in each case to one line of Table B.

Table 111ka Compounds of formula I.Ka in which o is 1, R⁷⁸ is 2″-Br, Y is C₂H₅ and the meaning for the combination of R¹, R², R³, R⁴, R⁹ and R¹⁰ for each individual compound corresponds in each case to one line of Table B.

Table 112ka Compounds of formula I.Ka in which o is 1, R⁷⁸ is 2″-CH₃, Y is C₂H₅ and the meaning for the combination of R¹, R², R³, R⁴, R⁹ and R¹⁰ for each individual compound corresponds in each case to one line of Table B.

Table 113ka Compounds of formula I.Ka in which o is 1, R⁷⁸ is 2″-OCH₃, Y is C₂H₅ and the meaning for the combination of R¹, R², R³, R⁴, R⁹ and R¹⁰ for each individual compound corresponds in each case to one line of Table B.

Table 114ka Compounds of formula I.Ka in which o is 1, R⁷⁸ is 2″-OCHF₂, Y is C₂H₅ and the meaning for the combination of R¹, R², R³, R⁴, R⁹ and R¹⁰ for each individual compound corresponds in each case to one line of Table B.

Table 115ka Compounds of formula I.Ka in which o is 1, R⁷⁸ is 2″-C₆H₅, Y is C₂H₅ and the meaning for the combination of R¹, R², R³, R⁴, R⁹ and R¹⁰ for each individual compound corresponds in each case to one line of Table B.

Table 116ka Compounds of formula I.Ka in which o is 1, R⁷⁸ is 3″-F, Y is C₂H₅ and the meaning for the combination of R¹, R², R³, R⁴, R⁹ and R¹⁰ for each individual compound corresponds in each case to one line of Table B.

Table 117ka Compounds of formula I.Ka in which o is 1, R⁷⁸ is 3″-Cl, Y is C₂H₅ and the meaning for the combination of R¹, R², R³, R⁴, R⁹ and R¹⁰ for each individual compound corresponds in each case to one line of Table B.

Table 118ka Compounds of formula I.Ka in which o is 1, R⁷⁸ is 3″-Br, Y is C₂H₅ and the meaning for the combination of R¹, R², R³, R⁴, R⁹ and R¹⁰ for each individual compound corresponds in each case to one line of Table B.

Table 119ka Compounds of formula I.Ka in which o is 1, R⁷⁸ is 3″-CH₃, Y is C₂H₅ and the meaning for the combination of R¹, R², R³, R⁴, R⁹ and R¹⁰ for each individual compound corresponds in each case to one line of Table B.

Table 120ka Compounds of formula I.Ka in which o is 1, R⁷⁸ is 3″-OCH₃, Y is C₂H₅ and the meaning for the combination of R¹, R², R³, R⁴, R⁹ and R¹⁰ for each individual compound corresponds in each case to one line of Table B.

Table 121ka Compounds of formula I.Ka in which o is 1, R⁷⁸ is 3″-OCHF₂, Y is C₂H₅ and the meaning for the combination of R¹, R², R³, R⁴, R⁹ and R¹⁰ for each individual compound corresponds in each case to one line of Table B.

Table 122ka Compounds of formula I.Ka in which o is 1, R⁷⁸ is 3″-C₆H₅, Y is C₂H₅ and the meaning for the combination of R¹, R², R³, R⁴, R⁹ and R¹⁰ for each individual compound corresponds in each case to one line of Table B.

le 123ka Compounds of formula I.Ka in which o is 0, Y is CHF₂ and the meaning for the combination of R¹, R², R³, R⁴, R⁹ and R¹⁰ for each individual compound corresponds in each case to one line of Table B.

Table 124ka Compounds of formula I.Ka in which o is 1, R⁷⁸ is 1″-F, Y is CHF₂ and the meaning for the combination of R¹, R², R³, R⁴, R⁹ and R¹⁰ for each individual compound corresponds in each case to one line of Table B.

Table 125ka Compounds of formula I.Ka in which o is 1, R⁷⁸ is 1″-Cl, Y is CHF₂ and the meaning for the combination of R¹, R², R³, R⁴, R⁹ and R¹⁰ for each individual compound corresponds in each case to one line of Table B.

Table 126ka Compounds of formula I.Ka in which o is 1, R⁷⁸ is 1″-Br, Y is CHF₂ and the meaning for the combination of R¹, R², R³, R⁴, R⁹ and R¹⁰ for each individual compound corresponds in each case to one line of Table B.

Table 127ka Compounds of formula I.Ka in which o is 1, R⁷⁸ is 1″-CH₃, Y is CHF₂ and the meaning for the combination of R¹, R², R³, R⁴, R⁹ and R¹⁰ for each individual compound corresponds in each case to one line of Table B.

Table 128ka Compounds of formula I.Ka in which o is 1, R⁷⁸ is 1″-OCH₃, Y is CHF₂ and the meaning for the combination of R¹, R², R³, R⁴, R⁹ and R¹⁰ for each individual compound corresponds in each case to one line of Table B.

Table 129ka Compounds of formula I.Ka in which o is 1, R⁷⁸ is 1″-OCHF₂, Y is CHF₂ and the meaning for the combination of R¹, R², R³, R⁴, R⁹ and R¹⁰ for each individual compound corresponds in each case to one line of Table B.

Table 130ka Compounds of formula I.Ka in which o is 1, R⁷⁸ is 1″-C₆H₅, Y is CHF₂ and the meaning for the combination of R¹, R², R³, R⁴, R⁹ and R¹⁰ for each individual compound corresponds in each case to one line of Table B.

Table 131ka Compounds of formula I.Ka in which o is 1, R⁷⁸ is 2″-F, Y is CHF₂ and the meaning for the combination of R¹, R², R³, R⁴, R⁹ and R¹⁰ for each individual compound corresponds in each case to one line of Table B.

Table 132ka Compounds of formula I.Ka in which o is 1, R⁷⁸ is 2″-Cl, Y is CHF₂ and the meaning for the combination of R¹, R², R³, R⁴, R⁹ and R¹⁰ for each individual compound corresponds in each case to one line of Table B.

Table 133ka Compounds of formula I.Ka in which o is 1, R⁷⁸ is 2″-Br, Y is CHF₂ and the meaning for the combination of R¹, R², R³, R⁴, R⁹ and R¹⁰ for each individual compound corresponds in each case to one line of Table B.

Table 134ka Compounds of formula I.Ka in which o is 1, R⁷⁸ is 2″-CH₃, Y is CHF₂ and the meaning for the combination of R¹, R², R³, R⁴, R⁹ and R¹⁰ for each individual compound corresponds in each case to one line of Table B.

Table 135ka Compounds of formula I.Ka in which o is 1, R⁷⁸ is 2″-OCH₃, Y is CHF₂ and the meaning for the combination of R¹, R², R³, R⁴, R⁹ and R¹⁰ for each individual compound corresponds in each case to one line of Table B.

Table 136ka Compounds of formula I.Ka in which o is 1, R⁷⁸ is 2″-OCHF₂, Y is CHF₂ and the meaning for the combination of R¹, R², R³, R⁴, R⁹ and R¹⁰ for each individual compound corresponds in each case to one line of Table B.

Table 137ka Compounds of formula I.Ka in which o is 1, R⁷⁸ is 2″-C₆H₅, Y is CHF₂ and the meaning for the combination of R¹, R², R³, R⁴, R⁹ and R¹⁰ for each individual compound corresponds in each case to one line of Table B.

Table 138ka Compounds of formula I.Ka in which o is 1, R⁷⁸ is 3″-F, Y is CHF₂ and the meaning for the combination of R¹, R², R³, R⁴, R⁹ and R¹⁰ for each individual compound corresponds in each case to one line of Table B.

Table 139ka Compounds of formula I.Ka in which o is 1, R⁷⁸ is 3″-Cl, Y is CHF₂ and the meaning for the combination of R¹, R², R³, R⁴, R⁹ and R¹⁰ for each individual compound corresponds in each case to one line of Table B.

Table 140ka Compounds of formula I.Ka in which o is 1, R⁷⁸ is 3″-Br, Y is CHF₂ and the meaning for the combination of R¹, R², R³, R⁴, R⁹ and R¹⁰ for each individual compound corresponds in each case to one line of Table B.

Table 141ka Compounds of formula I.Ka in which o is 1, R⁷⁸ is 3″-CH₃, Y is CHF₂ and the meaning for the combination of R¹, R², R³, R⁴, R⁹ and R¹⁰ for each individual compound corresponds in each case to one line of Table B.

Table 142ka Compounds of formula I.Ka in which o is 1, R⁷⁸ is 3″-OCH₃, Y is CHF₂ and the meaning for the combination of R¹, R², R³, R⁴, R⁹ and R¹⁰ for each individual compound corresponds in each case to one line of Table B.

Table 143ka Compounds of formula I.Ka in which o is 1, R⁷⁸ is 3″-OCHF₂, Y is CHF₂ and the meaning for the combination of R¹, R², R³, R⁴, R⁹ and R¹⁰ for each individual compound corresponds in each case to one line of Table B.

Table 144ka Compounds of formula I.Ka in which o is 1, R⁷⁸ is 3″-C₆H₅, Y is CHF₂ and the meaning for the combination of R¹, R², R³, R⁴, R⁹ and R¹⁰ for each individual compound corresponds in each case to one line of Table B.

Table 145ka Compounds of formula I.Ka in which o is 0, Y is CN and the meaning for the combination of R¹, R², R³, R⁴, R⁹ and R¹⁰ for each individual compound corresponds in each case to one line of Table B.

Table 146ka Compounds of formula I.Ka in which o is 1, R⁷⁸ is 3″-CHF₂, Y is CHF₂ and the meaning for the combination of R¹, R², R³, R⁴, R⁹ and R¹⁰ for each individual compound corresponds in each case to one line of Table B.

Table 147ka Compounds of formula I.Ka in which o is 1, R⁷⁸ is 3″-OCF₃, Y is CHF₂ and the meaning for the combination of R¹, R², R³, R⁴, R⁹ and R¹⁰ for each individual compound corresponds in each case to one line of Table B.

Table 148ka Compounds of formula I.Ka in which o is 1, R⁷⁸ is 3″-CF₃, Y is CHF₂ and the meaning for the combination of R¹, R², R³, R⁴, R⁹ and R¹⁰ for each individual compound corresponds in each case to one line of Table B.

Table 149ka Compounds of formula I.Ka in which o is 1, R⁷⁸ is 3″-CN Y is CHF₂ and the meaning for the combination of R¹, R², R³, R⁴, R⁹ and R¹⁰ for each individual compound corresponds in each case to one line of Table B.

Table 150ka Compounds of formula I.Ka in which o is 1, R⁷⁸ is 1″-F, Y is CN and the meaning for the combination of R¹, R², R³, R⁴, R⁹ and R¹⁰ for each individual compound corresponds in each case to one line of Table B.

Table 151ka Compounds of formula I.Ka in which o is 1, R⁷⁸ is 1″-Cl, Y is CN and the meaning for the combination of R¹, R², R³, R⁴, R⁹ and R¹⁰ for each individual compound corresponds in each case to one line of Table B.

Table 152ka Compounds of formula I.Ka in which o is 1, R⁷⁸ is 1″-Br, Y is CN and the meaning for the combination of R¹, R², R³, R⁴, R⁹ and R¹⁰ for each individual compound corresponds in each case to one line of Table B.

Table 153ka Compounds of formula I.Ka in which o is 1, R⁷⁸ is 1″-CH₃, Y is CN and the meaning for the combination of R¹, R², R³, R⁴, R⁹ and R¹⁰ for each individual compound corresponds in each case to one line of Table B.

Table 154ka Compounds of formula I.Ka in which o is 1, R⁷⁸ is 1″-OCH₃, Y is CN and the meaning for the combination of R¹, R², R³, R⁴, R⁹ and R¹⁰ for each individual compound corresponds in each case to one line of Table B.

Table 155ka Compounds of formula I.Ka in which o is 1, R⁷⁸ is 1″-OCHF₂, Y is CN and the meaning for the combination of R¹, R², R³, R⁴, R⁹ and R¹⁰ for each individual compound corresponds in each case to one line of Table B.

Table 156ka Compounds of formula I.Ka in which o is 1, R⁷⁸ is 1″-C₆H₅, Y is CN and the meaning for the combination of R¹, R², R³, R⁴, R⁹ and R¹⁰ for each individual compound corresponds in each case to one line of Table B.

Table 157ka Compounds of formula I.Ka in which o is 1, R⁷⁸ is 2″-F, Y is CN and the meaning for the combination of R¹, R², R³, R⁴, R⁹ and R¹⁰ for each individual compound corresponds in each case to one line of Table B.

Table 158ka Compounds of formula I.Ka in which o is 1, R⁷⁸ is 2″-Cl, Y is CN and the meaning for the combination of R¹, R², R³, R⁴, R⁹ and R¹⁰ for each individual compound corresponds in each case to one line of Table B.

Table 159ka Compounds of formula I.Ka in which o is 1, R⁷⁸ is 2″-Br, Y is CN and the meaning for the combination of R¹, R², R³, R⁴, R⁹ and R¹⁰ for each individual compound corresponds in each case to one line of Table B.

Table 160ka Compounds of formula I.Ka in which o is 1, R⁷⁸ is 2″-CH₃, Y is CN and the meaning for the combination of R¹, R², R³, R⁴, R⁹ and R¹⁰ for each individual compound corresponds in each case to one line of Table B.

Table 161ka Compounds of formula I.Ka in which o is 1, R⁷⁸ is 2″-OCH₃, Y is CN and the meaning for the combination of R¹, R², R³, R⁴, R⁹ and R¹⁰ for each individual compound corresponds in each case to one line of Table B.

Table 162ka Compounds of formula I.Ka in which o is 1, R⁷⁸ is 2″-OCHF₂, Y is CN and the meaning for the combination of R¹, R², R³, R⁴, R⁹ and R¹⁰ for each individual compound corresponds in each case to one line of Table B.

Table 163ka Compounds of formula I.Ka in which o is 1, R⁷⁸ is 2″-C₆H₅, Y is CN and the meaning for the combination of R¹, R², R³, R⁴, R⁹ and R¹⁰ for each individual compound corresponds in each case to one line of Table B.

Table 164ka Compounds of formula I.Ka in which o is 1, R⁷⁸ is 3″-F, Y is CN and the meaning for the combination of R¹, R², R³, R⁴, R⁹ and R¹⁰ for each individual compound corresponds in each case to one line of Table B.

Table 165ka Compounds of formula I.Ka in which o is 1, R⁷⁸ is 3″-Cl, Y is CN and the meaning for the combination of R¹, R², R³, R⁴, R⁹ and R¹⁰ for each individual compound corresponds in each case to one line of Table B.

Table 166ka Compounds of formula I.Ka in which o is 1, R⁷⁸ is 3″-Br, Y is CN and the mean-ing for the combination of R¹, R², R³, R⁴, R⁹ and R¹⁰ for each individual compound corresponds in each case to one line of Table B.

Table 167ka Compounds of formula I.Ka in which o is 1, R⁷⁸ is 3″-CH₃, Y is CN and the meaning for the combination of R¹, R², R³, R⁴, R⁹ and R¹⁰ for each individual compound corresponds in each case to one line of Table B.

Table 168ka Compounds of formula I.Ka in which o is 1, R⁷⁸ is 3″-OCH₃, Y is CN and the meaning for the combination of R¹, R², R³, R⁴, R⁹ and R¹⁰ for each individual compound corresponds in each case to one line of Table B.

Table 169ka Compounds of formula I.Ka in which o is 1, R⁷⁸ is 3″-OCHF₂, Y is CN and the meaning for the combination of R¹, R², R³, R⁴, R⁹ and R¹⁰ for each individual compound corresponds in each case to one line of Table B.

Table 170ka Compounds of formula I.Ka in which o is 1, R⁷⁸ is 3″-C₆H₅, Y is CN and the meaning for the combination of R¹, R², R³, R⁴, R⁹ and R¹⁰ for each individual compound corresponds in each case to one line of Table B.

Table 171ka Compounds of formula I.Ka in which o is 0, Y is OCHF₂ and the meaning for the combination of R¹, R², R³, R⁴, R⁹ and R¹⁰ for each individual compound corresponds in each case to one line of Table B.

Table 172ka Compounds of formula I.Ka in which o is 1, R⁷⁸ is 1″-F, Y is OCHF₂ and the meaning for the combination of R¹, R², R³, R⁴, R⁹ and R¹⁰ for each individual compound corresponds in each case to one line of Table B.

Table 173ka Compounds of formula I.Ka in which o is 1, R⁷⁸ is 1″-Cl, Y is OCHF₂ and the meaning for the combination of R¹, R², R³, R⁴, R⁹ and R¹⁰ for each individual compound corresponds in each case to one line of Table B.

Table 174ka Compounds of formula I.Ka in which o is 1, R⁷⁸ is 1″-Br, Y is OCHF₂ and the meaning for the combination of R¹, R², R³, R⁴, R⁹ and R¹⁰ for each individual compound corresponds in each case to one line of Table B.

Table 175ka Compounds of formula I.Ka in which o is 1, R⁷⁸ is 1″-CH₃, Y is OCHF₂ and the meaning for the combination of R¹, R², R³, R⁴, R⁹ and R¹⁰ for each individual compound corresponds in each case to one line of Table B.

Table 176ka Compounds of formula I.Ka in which o is 1, R⁷⁸ is 1″-OCH₃, Y is OCHF₂ and the meaning for the combination of R¹, R², R³, R⁴, R⁹ and R¹⁰ for each individual compound corresponds in each case to one line of Table B.

Table 177ka Compounds of formula I.Ka in which o is 1, R⁷⁸ is 1″-OCHF₂, Y is OCHF₂ and the meaning for the combination of R¹, R², R³, R⁴, R⁹ and R¹⁰ for each individual compound corresponds in each case to one line of Table B.

Table 178ka Compounds of formula I.Ka in which o is 1, R⁷⁸ is 1″-C₆H₅, Y is OCHF₂ and the meaning for the combination of R¹, R², R³, R⁴, R⁹ and R¹⁰ for each individual compound corresponds in each case to one line of Table B.

Table 179ka Compounds of formula I.Ka in which o is 1, R⁷⁸ is 2″-F, Y is OCHF₂ and the meaning for the combination of R¹, R², R³, R⁴, R⁹ and R¹⁰ for each individual compound corresponds in each case to one line of Table B.

Table 180ka Compounds of formula I.Ka in which o is 1, R⁷⁸ is 2″-Cl, Y is OCHF₂ and the meaning for the combination of R¹, R², R³, R⁴, R⁹ and R¹⁰ for each individual compound corresponds in each case to one line of Table B.

Table 181ka Compounds of formula I.Ka in which o is 1, R⁷⁸ is 2″-Br, Y is OCHF₂ and the meaning for the combination of R¹, R², R³, R⁴, R⁹ and R¹⁰ for each individual compound corresponds in each case to one line of Table B.

Table 182ka Compounds of formula I.Ka in which o is 1, R⁷⁸ is 2″-CH₃, Y is OCHF₂ and the meaning for the combination of R¹, R², R³, R⁴, R⁹ and R¹⁰ for each individual compound corre-sponds in each case to one line of Table B.

Table 183ka Compounds of formula I.Ka in which o is 1, R⁷⁸ is 2″-OCH₃, Y is OCHF₂ and the meaning for the combination of R¹, R², R³, R⁴, R⁹ and R¹⁰ for each individual compound corresponds in each case to one line of Table B.

Table 184ka Compounds of formula I.Ka in which o is 1, R⁷⁸ is 2″-OCHF₂, Y is OCHF₂ and the meaning for the combination of R¹, R², R³, R⁴, R⁹ and R¹⁰ for each individual compound corresponds in each case to one line of Table B.

Table 185ka Compounds of formula I.Ka in which o is 1, R⁷⁸ is 2″-C₆H₅, Y is OCHF₂ and the meaning for the combination of R¹, R², R³, R⁴, R⁹ and R¹⁰ for each individual compound corresponds in each case to one line of Table B.

Table 186ka Compounds of formula I.Ka in which o is 1, R⁷⁸ is 3″-F, Y is OCHF₂ and the meaning for the combination of R¹, R², R³, R⁴, R⁹ and R¹⁰ for each individual compound corresponds in each case to one line of Table B.

Table 187ka Compounds of formula I.Ka in which o is 1, R⁷⁸ is 3″-Cl, Y is OCHF₂ and the meaning for the combination of R¹, R², R³, R⁴, R⁹ and R¹⁰ for each individual compound corresponds in each case to one line of Table B.

Table 188ka Compounds of formula I.Ka in which o is 1, R⁷⁸ is 3″-Br, Y is OCHF₂ and the meaning for the combination of R¹, R², R³, R⁴, R⁹ and R¹⁰ for each individual compound corresponds in each case to one line of Table B.

Table 189ka Compounds of formula I.Ka in which o is 1, R⁷⁸ is 3″-CH₃, Y is OCHF₂ and the meaning for the combination of R¹, R², R³, R⁴, R⁹ and R¹⁰ for each individual compound corresponds in each case to one line of Table B.

Table 190ka Compounds of formula I.Ka in which o is 1, R⁷⁸ is 3″-OCH₃, Y is OCHF₂ and the meaning for the combination of R¹, R², R³, R⁴, R⁹ and R¹⁰ for each individual compound corresponds in each case to one line of Table B.

Table 191ka Compounds of formula I.Ka in which o is 1, R⁷⁸ is 3″-OCHF₂, Y is OCHF₂ and the meaning for the combination of R¹, R², R³, R⁴, R⁹ and R¹⁰ for each individual compound corresponds in each case to one line of Table B.

Table 192ka Compounds of formula I.Ka in which o is 1, R⁷⁸ is 3″-C₆H₅, Y is OCHF₂ and the meaning for the combination of R¹, R², R³, R⁴, R⁹ and R¹⁰ for each individual compound corresponds in each case to one line of Table B.

Table 193ka Compounds of formula I.Ka in which o is 1, R⁷⁸ is 3″-CHF₂, Y is OCHF₂ and the meaning for the combination of R¹, R², R³, R⁴, R⁹ and R¹⁰ for each individual compound corresponds in each case to one line of Table B.

Table 194ka Compounds of formula I.Ka in which o is 1, R⁷⁸ is 3″-OCF₃, Y is OCHF₂ and the meaning for the combination of R¹, R², R³, R⁴, R⁹ and R¹⁰ for each individual compound corresponds in each case to one line of Table B.

Table 195ka Compounds of formula I.Ka in which o is 1, R⁷⁸ is 3″-CF₃, Y is OCHF₂ and the meaning for the combination of R¹, R², R³, R⁴, R⁹ and R¹⁰ for each individual compound corresponds in each case to one line of Table B.

Table 196ka Compounds of formula I.Ka in which o is 1, R⁷⁸ is 3″-CN Y is OCHF₂ and the meaning for the combination of R¹, R², R³, R⁴, R⁹ and R¹⁰ for each individual compound corresponds in each case to one line of Table B.

Table 197ka Compounds of formula I.Ka in which o is 0, Y is Cl and the meaning for the combination of R¹, R², R³, R⁴, R⁹ and R¹⁰ for each individual compound corresponds in each case to one line of Table B.

Table 198ka Compounds of formula I.Ka in which o is 1, R⁷⁸ is 1″-F, Y is Cl and the meaning for the combination of R¹, R², R³, R⁴, R⁹ and R¹⁰ for each individual compound corresponds in each case to one line of Table B.

Table 199ka Compounds of formula I.Ka in which o is 1, R⁷⁸ is 1″-Cl, Y is Cl and the meaning for the combination of R¹, R², R³, R⁴, R⁹ and R¹⁰ for each individual compound corresponds in each case to one line of Table B.

Table 200ka Compounds of formula I.Ka in which o is 1, R⁷⁸ is 1″-Br, Y is Cl and the meaming for the combination of R¹, R², R³, R⁴, R⁹ and R¹⁰ for each individual compound corresponds in each case to one line of Table B.

Table 201ka Compounds of formula I.Ka in which o is 1, R⁷⁸ is 1″-CH₃, Y is Cl and the meaning for the combination of R¹, R², R³, R⁴, R⁹ and R¹⁰ for each individual compound corresponds in each case to one line of Table B.

Table 202ka Compounds of formula I.Ka in which o is 1, R⁷⁸ is 1″-OCH₃, Y is Cl and the meaning for the combination of R¹, R², R³, R⁴, R⁹ and R¹⁰ for each individual compound corresponds in each case to one line of Table B.

Table 203ka Compounds of formula I.Ka in which o is 1, R⁷⁸ is 1″-OCHF₂, Y is Cl and the meaning for the combination of R¹, R², R³, R⁴, R⁹ and R¹⁰ for each individual compound corresponds in each case to one line of Table B.

Table 204ka Compounds of formula I.Ka in which o is 1, R⁷⁸ is 1″-C₆H₅, Y is Cl and the meaning for the combination of R¹, R², R³, R⁴, R⁹ and R¹⁰ for each individual compound corresponds in each case to one line of Table B.

Table 205ka Compounds of formula I.Ka in which o is 1, R⁷⁸ is 2″-F, Y is Cl and the meaning for the combination of R¹, R², R³, R⁴, R⁹ and R¹⁰ for each individual compound corresponds in each case to one line of Table B.

Table 206ka Compounds of formula I.Ka in which o is 1, R⁷⁸ is 2″-Cl, Y is Cl and the meaning for the combination of R¹, R², R³, R⁴, R⁹ and R¹⁰ for each individual compound corresponds in each case to one line of Table B.

Table 207ka Compounds of formula I.Ka in which o is 1, R⁷⁸ is 2″-Br, Y is Cl and the meaning for the combination of R¹, R², R³, R⁴, R⁹ and R¹⁰ for each individual compound corresponds in each case to one line of Table B.

Table 208ka Compounds of formula I.Ka in which o is 1, R⁷⁸ is 2″-CH₃, Y is Cl and the meaning for the combination of R¹, R², R³, R⁴, R⁹ and R¹⁰ for each individual compound corresponds in each case to one line of Table B.

Table 209ka Compounds of formula I.Ka in which o is 1, R⁷⁸ is 2″-OCH₃, Y is Cl and the meaning for the combination of R¹, R², R³, R⁴, R⁹ and R¹⁰ for each individual compound corresponds in each case to one line of Table B.

Table 210ka Compounds of formula I.Ka in which o is 1, R⁷⁸ is 2″-OCHF₂, Y is Cl and the meaning for the combination of R¹, R², R³, R⁴, R⁹ and R¹⁰ for each individual compound corresponds in each case to one line of Table B.

Table 211ka Compounds of formula I.Ka in which o is 1, R⁷⁸ is 2″-C₆H₅, Y is Cl and the meaning for the combination of R¹, R², R³, R⁴, R⁹ and R¹⁰ for each individual compound corresponds in each case to one line of Table B.

Table 212ka Compounds of formula I.Ka in which o is 1, R⁷⁸ is 3″-F, Y is Cl and the meaning for the combination of R¹, R², R³, R⁴, R⁹ and R¹⁰ for each individual compound corresponds in each case to one line of Table B.

Table 213ka Compounds of formula I.Ka in which o is 1, R⁷⁸ is 3″-Cl, Y is Cl and the meaning for the combination of R¹, R², R³, R⁴, R⁹ and R¹⁰ for each individual compound corresponds in each case to one line of Table B.

Table 214ka Compounds of formula I.Ka in which o is 1, R⁷⁸ is 3″-Br, Y is Cl and the meaning for the combination of R¹, R², R³, R⁴, R⁹ and R¹⁰ for each individual compound corresponds in each case to one line of Table B.

Table 215ka Compounds of formula I.Ka in which o is 1, R⁷⁸ is 3″-CH₃, Y is Cl and the meaning for the combination of R¹, R², R³, R⁴, R⁹ and R¹⁰ for each individual compound corresponds in each case to one line of Table B.

Table 216ka Compounds of formula I.Ka in which o is 1, R⁷⁸ is 3″-OCH₃, Y is Cl and the meaning for the combination of R¹, R², R³, R⁴, R⁹ and R¹⁰ for each individual compound corresponds in each case to one line of Table B.

Table 217ka Compounds of formula I.Ka in which o is 1, R⁷⁸ is 3″-OCHF₂, Y is Cl and the meaning for the combination of R¹, R², R³, R⁴, R⁹ and R¹⁰ for each individual compound corresponds in each case to one line of Table B.

Table 218ka Compounds of formula I.Ka in which o is 1, R⁷⁸ is 3″-C₆H₅, Y is Cl and the meaning for the combination of R¹, R², R³, R⁴, R⁹ and R¹⁰ for each individual compound corresponds in each case to one line of Table B.

Table 219ka Compounds of formula I.Ka in which o is 1, R⁷⁸ is 3″-CHF₂, Y is Cl and the meaning for the combination of R¹, R², R³, R⁴, R⁹ and R¹⁰ for each individual compound corresponds in each case to one line of Table B.

Table 220ka Compounds of formula I.Ka in which o is 1, R⁷⁸ is 3″-OCF₃, Y is Cl and the meaning for the combination of R¹, R², R³, R⁴, R⁹ and R¹⁰ for each individual compound corresponds in each case to one line of Table B.

Table 221ka Compounds of formula I.Ka in which o is 1, R⁷⁸ is 3″-CF₃, Y is Cl and the meaning for the combination of R¹, R², R³, R⁴, R⁹ and R¹⁰ for each individual compound corresponds in each case to one line of Table B.

Table 222ka Compounds of formula I.Ka in which o is 1, R⁷⁸ is 3″-CN Y is Cl and the meaning for the combination of R¹, R², R³, R⁴, R⁹ and R¹⁰ for each individual compound corresponds in each case to one line of Table B.

Table 223ka Compounds of formula I.Ka in which o is 0, Y is OMe and the meaning for the combination of R¹, R², R³, R⁴, R⁹ and R¹⁰ for each individual compound corresponds in each case to one line of Table B.

Table 224ka Compounds of formula I.Ka in which o is 1, R⁷⁸ is 1″-F, Y is OMe and the meaning for the combination of R¹, R², R³, R⁴, R⁹ and R¹⁰ for each individual compound corresponds in each case to one line of Table B.

Table 225ka Compounds of formula I.Ka in which o is 1, R⁷⁸ is 1″-Cl, Y is OMe and the meaning for the combination of R¹, R², R³, R⁴, R⁹ and R¹⁰ for each individual compound corresponds in each case to one line of Table B.

Table 226ka Compounds of formula I.Ka in which o is 1, R⁷⁸ is 1″-Br, Y is OMe and the meaning for the combination of R¹, R², R³, R⁴, R⁹ and R¹⁰ for each individual compound corresponds in each case to one line of Table B.

Table 227ka Compounds of formula I.Ka in which o is 1, R⁷⁸ is 1″-CH₃₇ Y is OMe and the meaning for the combination of R¹, R², R³, R⁴, R⁹ and R¹⁰ for each individual compound corresponds in each case to one line of Table B.

Table 228ka Compounds of formula I.Ka in which o is 1, R⁷⁸ is 1″-OCH₃, Y is OMe and the meaning for the combination of R¹, R², R³, R⁴, R⁹ and R¹⁰ for each individual compound corresponds in each case to one line of Table B.

Table 229ka Compounds of formula I.Ka in which o is 1, R⁷⁸ is 1″-OCHF₂, Y is OMe and the meaning for the combination of R¹, R², R³, R⁴, R⁹ and R¹⁰ for each individual compound corresponds in each case to one line of Table B.

Table 230ka Compounds of formula I.Ka in which o is 1, R⁷⁸ is 1″-C₆H₅, Y is OMe and the meaning for the combination of R¹, R², R³, R⁴, R⁹ and R¹⁰ for each individual compound corresponds in each case to one line of Table B.

Table 231ka Compounds of formula I.Ka in which o is 1, R⁷⁸ is 2″-F, Y is OMe and the meaning for the combination of R¹, R², R³, R⁴, R⁹ and R¹⁰ for each individual compound corresponds in each case to one line of Table B.

Table 232ka Compounds of formula I.Ka in which o is 1, R⁷⁸ is 2″-Cl, Y is OMe and the meaning for the combination of R¹, R², R³, R⁴, R⁹ and R¹⁰ for each individual compound corresponds in each case to one line of Table B.

Table 233ka Compounds of formula I.Ka in which o is 1, R⁷⁸ is 2″-Br, Y is OMe and the meaning for the combination of R¹, R², R³, R⁴, R⁹ and R¹⁰ for each individual compound corresponds in each case to one line of Table B.

Table 234ka Compounds of formula I.Ka in which o is 1, R⁷⁸ is 2″-CH₃₇ Y is OMe and the meaning for the combination of R¹, R², R3_{,} R4, R⁹ and R¹⁰ for each individual compound corresponds in each case to one line of Table B.

Table 235ka Compounds of formula I.Ka in which o is 1, R⁷⁸ is 2″-OCH₃, Y is OMe and the meaning for the combination of R¹, R², R3_{,} R4, R⁹ and R¹⁰ for each individual compound corresponds in each case to one line of Table B.

Table 236ka Compounds of formula I.Ka in which o is 1, R⁷⁸ is 2″-OCH F₂, Y is OMe and the meaning for the combination of R¹, R², R3_{,} R4, R⁹ and R¹⁰ for each individual compound corresponds in each case to one line of Table B.

Table 237ka Compounds of formula I.Ka in which o is 1, R⁷⁸ is 2″-C₆H₅, Y is OMe and the meaning for the combination of R¹, R², R3_{,} R4, R⁹ and R¹⁰ for each individual compound corresponds in each case to one line of Table B.

Table 238ka Compounds of formula I.Ka in which o is 1, R⁷⁸ is 3″-F, Y is OMe and the meaning for the combination of R¹, R², R3_{,} R4, R⁹ and R¹⁰ for each individual compound corresponds in each case to one line of Table B.

Table 239ka Compounds of formula I.Ka in which o is 1, R⁷⁸ is 3″-Cl, Y is OMe and the meaning for the combination of R¹, R², R3_{,} R4, R⁹ and R¹⁰ for each individual compound corresponds in each case to one line of Table B.

Table 240ka Compounds of formula I.Ka in which o is 1, R⁷⁸ is 3″-Br, Y is OMe and the meaning for the combination of R¹, R², R3_{,} R4, R⁹ and R¹⁰ for each individual compound corresponds in each case to one line of Table B.

Table 241ka Compounds of formula I.Ka in which o is 1, R⁷⁸ is 3″-CH₃, Y is OMe and the meaning for the combination of R¹, R², R3_{,} R4, R⁹ and R¹⁰ for each individual compound corresponds in each case to one line of Table B.

Table 242ka Compounds of formula I.Ka in which o is 1, R⁷⁸ is 3″-OCH₃, Y is OMe and the meaning for the combination of R¹, R², R3_{,} R4, R⁹ and R¹⁰ for each individual compound corresponds in each case to one line of Table B.

Table 243ka Compounds of formula I.Ka in which o is 1, R⁷⁸ is 3″-OCH F₂, Y is OMe and the meaning for the combination of R¹, R², R3_{,} R4, R⁹ and R¹⁰ for each individual compound corresponds in each case to one line of Table B.

Table 244ka Compounds of formula I.Ka in which o is 1, R⁷⁸ is 3″-C₆H₅, Y is OMe and the meaning for the combination of R¹, R², R3_{,} R4, R⁹ and R¹⁰ for each individual compound corresponds in each case to one line of Table B.

Table 245ka Compounds of formula I.Ka in which o is 1, R⁷⁸ is 3″-CHF₂, Y is OMe and the meaning for the combination of R¹, R², R3_{,} R4, R⁹ and R¹⁰ for each individual compound corresponds in each case to one line of Table B.

Table 246ka Compounds of formula I.Ka in which o is 1, R⁷⁸ is 3″-OCF₃, Y is OMe and the meaning for the combination of R¹, R², R3_{,} R4, R⁹ and R¹⁰ for each individual compound corresponds in each case to one line of Table B.

Table 247ka Compounds of formula I.Ka in which o is 1, R⁷⁸ is 3″-CF₃, Y is OMe and the meaning for the combination of R¹, R², R3_{,} R4, R⁹ and R¹⁰ for each individual compound corresponds in each case to one line of Table B.

Table 248ka Compounds of formula I.Ka in which o is 1, R⁷⁸ is 3″-CN Y is OMe and the meaning for the combination of R¹, R², R3_{,} R4, R⁹ and R¹⁰ for each individual compound corresponds in each case to one line of Table B.

In Table B the following abbreviations are used:

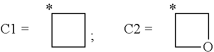

TABLE B

| line | R¹ | R² | R³ | R⁴ | R⁹ | R¹⁰ |
|---|---|---|---|---|---|---|
| B-1 | H | H | CH₃ | CH₃ | CH₃ | CH₃ |
| B-2 | H | H | CH₃ | CH₃ | CH₃ | CHF₂ |
| B-3 | H | H | CH₃ | CH₃ | CHF₂ | CH₃ |
| B-4 | H | H | CH₃ | CH₃ | CH₃ | C≡CH |
| B-5 | H | H | CH₃ | CH₃ | C≡CH | CH₃ |
| B-6 | H | H | CH₃ | CH₃ | CH₂F | CH₃ |
| B-7 | H | H | CH₃ | CH₃ | CH₃ | CH₂F |
| B-8 | H | H | CH₃ | CH₃ | CH₂F | Br |
| B-9 | H | H | CH₃ | CH₃ | Br | CH₂F |
| B-10 | H | H | CH₃ | CH₃ | OCH₃ | CHF₂ |
| B-11 | H | H | CH₃ | CH₃ | CHF₂ | OCH₃ |
| B-12 | H | H | CHF₂ | CH₃ | CH₃ | CH₃ |
| B-13 | H | H | CHF₂ | CH₃ | CH₃ | CHF₂ |
| B-14 | H | H | CHF₂ | CH₃ | CHF₂ | CH₃ |
| B-15 | H | H | CHF₂ | CH₃ | CH₃ | C≡CH |
| B-16 | H | H | CHF₂ | CH₃ | C≡CH | CH₃ |
| B-17 | H | H | CHF₂ | CH₃ | CH₂F | CH₃ |
| B-18 | H | H | CHF₂ | CH₃ | CH₃ | CH₂F |
| B-19 | H | H | CHF₂ | CH₃ | CH₂F | Br |
| B-20 | H | H | CHF₂ | CH₃ | Br | CH₂F |
| B-21 | H | H | CHF₂ | CH₃ | OCH₃ | CHF₂ |
| B-22 | H | H | CHF₂ | CH₃ | CHF₂ | OCH₃ |
| B-23 | H | H | CF₃ | CH₃ | CH₃ | CH₃ |
| B-24 | H | H | CF₃ | CH₃ | CH₃ | CHF₂ |
| B-25 | H | H | CF₃ | CH₃ | CHF₂ | CH₃ |
| B-26 | H | H | CF₃ | CH₃ | CH₃ | C≡CH |
| B-27 | H | H | CF₃ | CH₃ | C≡CH | CH₃ |
| B-28 | H | H | CF₃ | CH₃ | CH₂F | CH₃ |
| B-29 | H | H | CF₃ | CH₃ | CH₃ | CH₂F |
| B-30 | H | H | CF₃ | CH₃ | CH₂F | Br |
| B-31 | H | H | CF₃ | CH₃ | Br | CH₂F |
| B-32 | H | H | CF₃ | CH₃ | OCH₃ | CHF₂ |
| B-33 | H | H | CF₃ | CH₃ | CHF₂ | OCH₃ |
| B-34 | H | H | CH₃ | CF₃ | CH₃ | CH₃ |
| B-35 | H | H | CH₃ | CF₃ | CH₃ | CHF₂ |
| B-36 | H | H | CH₃ | CF₃ | CHF₂ | CH₃ |
| B-37 | H | H | CH₃ | CF₃ | CH₃ | C≡CH |
| B-38 | H | H | CH₃ | CF₃ | C≡CH | CH₃ |
| B-39 | H | H | CH₃ | CF₃ | CH₂F | CH₃ |
| B-40 | H | H | CH₃ | CF₃ | CH₃ | CH₂F |
| B-41 | H | H | CH₃ | CF₃ | CH₂F | Br |
| B-42 | H | H | CH₃ | CF₃ | Br | CH₂F |
| B-43 | H | H | CH₃ | CF₃ | OCH₃ | CHF₂ |
| B-44 | H | H | CH₃ | CF₃ | CHF₂ | OCH₃ |
| B-45 | H | H | CHF₂ | CF₃ | CH₃ | CH₃ |
| B-46 | H | H | CHF₂ | CF₃ | CH₃ | CHF₂ |
| B-47 | H | H | CHF₂ | CF₃ | CHF₂ | CH₃ |
| B-48 | H | H | CHF₂ | CF₃ | CH₃ | C≡CH |
| B-49 | H | H | CHF₂ | CF₃ | C≡CH | CH₃ |
| B-50 | H | H | CHF₂ | CF₃ | CH₂F | CH₃ |
| B-51 | H | H | CHF₂ | CF₃ | CH₃ | CH₂F |
| B-52 | H | H | CHF₂ | CF₃ | CH₂F | Br |
| B-53 | H | H | CHF₂ | CF₃ | Br | CH₂F |
| B-54 | H | H | CHF₂ | CF₃ | OCH₃ | CHF₂ |
| B-55 | H | H | CHF₂ | CF₃ | CHF₂ | OCH₃ |
| B-56 | H | H | CF₃ | CF₃ | CH₃ | CH₃ |
| B-57 | H | H | CF₃ | CF₃ | CH₃ | CHF₂ |
| B-58 | H | H | CF₃ | CF₃ | CHF₂ | CH₃ |
| B-59 | H | H | CF₃ | CF₃ | CH₃ | C≡CH |
| B-60 | H | H | CF₃ | CF₃ | C≡CH | CH₃ |
| B-61 | H | H | CF₃ | CF₃ | CH₂F | CH₃ |
| B-62 | H | H | CF₃ | CF₃ | CH₃ | CH₂F |
| B-63 | H | H | CF₃ | CF₃ | CH₂F | Br |
| B-64 | H | H | CF₃ | CF₃ | Br | CH₂F |
| B-65 | H | H | CF₃ | CF₃ | OCH₃ | CHF₂ |
| B-66 | H | H | CF₃ | CF₃ | CHF₂ | OCH₃ |
| B-67 | H | H | CH₃ | CHF₂ | CH₃ | CH₃ |
| B-68 | H | H | CH₃ | CHF₂ | CH₃ | CHF₂ |
| B-69 | H | H | CH₃ | CHF₂ | CHF₂ | CH₃ |
| B-70 | H | H | CH₃ | CHF₂ | CH₃ | C≡CH |
| B-71 | H | H | CH₃ | CHF₂ | C≡CH | CH₃ |
| B-72 | H | H | CH₃ | CHF₂ | CH₂F | CH₃ |
| B-73 | H | H | CH₃ | CHF₂ | CH₃ | CH₂F |
| B-74 | H | H | CH₃ | CHF₂ | CH₂F | Br |
| B-75 | H | H | CH₃ | CHF₂ | Br | CH₂F |
| B-76 | H | H | CH₃ | CHF₂ | OCH₃ | CHF₂ |
| B-77 | H | H | CH₃ | CHF₂ | CHF₂ | OCH₃ |
| B-78 | H | H | CHF₂ | CHF₂ | CH₃ | CH₃ |
| B-79 | H | H | CHF₂ | CHF₂ | CH₃ | CHF₂ |
| B-80 | H | H | CHF₂ | CHF₂ | CHF₂ | CH₃ |
| B-81 | H | H | CHF₂ | CHF₂ | CH₃ | C≡CH |
| B-82 | H | H | CHF₂ | CHF₂ | C≡CH | CH₃ |
| B-83 | H | H | CHF₂ | CHF₂ | CH₂F | CH₃ |
| B-84 | H | H | CHF₂ | CHF₂ | CH₃ | CH₂F |
| B-85 | H | H | CHF₂ | CHF₂ | CH₂F | Br |
| B-86 | H | H | CHF₂ | CHF₂ | Br | CH₂F |
| B-87 | H | H | CHF₂ | CHF₂ | OCH₃ | CHF₂ |
| B-88 | H | H | CHF₂ | CHF₂ | CHF₂ | OCH₃ |
| B-89 | H | H | CF₃ | CHF₂ | CH₃ | CH₃ |
| B-90 | H | H | CF₃ | CHF₂ | CH₃ | CHF₂ |
| B-91 | H | H | CF₃ | CHF₂ | CHF₂ | CH₃ |
| B-92 | H | H | CF₃ | CHF₂ | CH₃ | C≡CH |
| B-93 | H | H | CF₃ | CHF₂ | C≡CH | CH₃ |
| B-94 | H | H | CF₃ | CHF₂ | CH₂F | CH₃ |
| B-95 | H | H | CF₃ | CHF₂ | CH₃ | CH₂F |
| B-96 | H | H | CF₃ | CHF₂ | CH₂F | Br |
| B-97 | H | H | CF₃ | CHF₂ | Br | CH₂F |
| B-98 | H | H | CF₃ | CHF₂ | OCH₃ | CHF₂ |
| B-99 | H | H | CF₃ | CHF₂ | CHF₂ | OCH₃ |
| B-100 | H | H | CH₃ | CH₂F | CH₃ | CH₃ |
| B-101 | H | H | CH₃ | CH₂F | CH₃ | CHF₂ |
| B-102 | H | H | CH₃ | CH₂F | CHF₂ | CH₃ |
| B-103 | H | H | CH₃ | CH₂F | CH₃ | C≡CH |
| B-104 | H | H | CH₃ | CH₂F | C≡CH | CH₃ |
| B-105 | H | H | CH₃ | CH₂F | CH₂F | CH₃ |
| B-106 | H | H | CH₃ | CH₂F | CH₃ | CH₂F |
| B-107 | H | H | CH₃ | CH₂F | CH₂F | Br |
| B-108 | H | H | CH₃ | CH₂F | Br | CH₂F |
| B-109 | H | H | CH₃ | CH₂F | OCH₃ | CHF₂ |
| B-110 | H | H | CH₃ | CH₂F | CHF₂ | OCH₃ |
| B-111 | H | H | CHF₂ | CH₂F | CH₃ | CH₃ |
| B-112 | H | H | CHF₂ | CH₂F | CH₃ | CHF₂ |
| B-113 | H | H | CHF₂ | CH₂F | CHF₂ | CH₃ |
| B-114 | H | H | CHF₂ | CH₂F | CH₃ | C≡CH |
| B-115 | H | H | CHF₂ | CH₂F | C≡CH | CH₃ |
| B-116 | H | H | CHF₂ | CH₂F | CH₂F | CH₃ |
| B-117 | H | H | CHF₂ | CH₂F | CH₃ | CH₂F |
| B-118 | H | H | CHF₂ | CH₂F | CH₂F | Br |
| B-119 | H | H | CHF₂ | CH₂F | Br | CH₂F |
| B-120 | H | H | CHF₂ | CH₂F | OCH₃ | CHF₂ |
| B-121 | H | H | CHF₂ | CH₂F | CHF₂ | OCH₃ |
| B-122 | H | H | CF₃ | CH₂F | CH₃ | CH₃ |
| B-123 | H | H | CF₃ | CH₂F | CH₃ | CHF₂ |
| B-124 | H | H | CF₃ | CH₂F | CHF₂ | CH₃ |
| B-125 | H | H | CF₃ | CH₂F | CH₃ | C≡CH |
| B-126 | H | H | CF₃ | CH₂F | C≡CH | CH₃ |
| B-127 | H | H | CF₃ | CH₂F | CH₂F | CH₃ |
| B-128 | H | H | CF₃ | CH₂F | CH₃ | CH₂F |
| B-129 | H | H | CF₃ | CH₂F | CH₂F | Br |
| B-130 | H | H | CF₃ | CH₂F | Br | CH₂F |
| B-131 | H | H | CF₃ | CH₂F | OCH₃ | CHF₂ |
| B-132 | H | H | CF₃ | CH₂F | CHF₂ | OCH₃ |
| B-133 | H | H | CH₃ | CN | CH₃ | CH₃ |
| B-134 | H | H | CH₃ | CN | CH₃ | CHF₂ |
| B-135 | H | H | CH₃ | CN | CHF₂ | CH₃ |
| B-136 | H | H | CH₃ | CN | CH₃ | C≡CH |
| B-137 | H | H | CH₃ | CN | C≡CH | CH₃ |
| B-138 | H | H | CH₃ | CN | CH₂F | CH₃ |
| B-139 | H | H | CH₃ | CN | CH₃ | CH₂F |
| B-140 | H | H | CH₃ | CN | CH₂F | Br |
| B-141 | H | H | CH₃ | CN | Br | CH₂F |
| B-142 | H | H | CH₃ | CN | OCH₃ | CHF₂ |
| B-143 | H | H | CH₃ | CN | CHF₂ | OCH₃ |
| B-144 | H | H | CHF₂ | CN | CH₃ | CH₃ |
| B-145 | H | H | CHF₂ | CN | CH₃ | CHF₂ |
| B-146 | H | H | CHF₂ | CN | CHF₂ | CH₃ |
| B-147 | H | H | CHF₂ | CN | CH₃ | C≡CH |
| B-148 | H | H | CHF₂ | CN | C≡CH | CH₃ |
| B-149 | H | H | CHF₂ | CN | CH₂F | CH₃ |
| B-150 | H | H | CHF₂ | CN | CH₃ | CH₂F |
| B-151 | H | H | CHF₂ | CN | CH₂F | Br |
| B-152 | H | H | CHF₂ | CN | Br | CH₂F |
| B-153 | H | H | CHF₂ | CN | OCH₃ | CHF₂ |
| B-154 | H | H | CHF₂ | CN | CHF₂ | OCH₃ |
| B-155 | H | H | CF₃ | CN | CH₃ | CH₃ |
| B-156 | H | H | CF₃ | CN | CH₃ | CHF₂ |
| B-157 | H | H | CF₃ | CN | CHF₂ | CH₃ |
| B-158 | H | H | CF₃ | CN | CH₃ | C≡CH |
| B-159 | H | H | CF₃ | CN | C≡CH | CH₃ |
| B-160 | H | H | CF₃ | CN | CH₂F | CH₃ |
| B-161 | H | H | CF₃ | CN | CH₃ | CH₂F |
| B-162 | H | H | CF₃ | CN | CH₂F | Br |
| B-163 | H | H | CF₃ | CN | Br | CH₂F |
| B-164 | H | H | CF₃ | CN | OCH₃ | CHF₂ |
| B-165 | H | H | CF₃ | CN | CHF₂ | OCH₃ |
| B-166 | H | H | CH₃ | CH₂CN | CH₃ | CH₃ |
| B-167 | H | H | CH₃ | CH₂CN | CH₃ | CHF₂ |
| B-168 | H | H | CH₃ | CH₂CN | CHF₂ | CH₃ |
| B-169 | H | H | CH₃ | CH₂CN | CH₃ | C≡CH |
| B-170 | H | H | CH₃ | CH₂CN | C≡CH | CH₃ |
| B-171 | H | H | CH₃ | CH₂CN | CH₂F | CH₃ |
| B-172 | H | H | CH₃ | CH₂CN | CH₃ | CH₂F |
| B-173 | H | H | CH₃ | CH₂CN | CH₂F | Br |
| B-174 | H | H | CH₃ | CH₂CN | Br | CH₂F |
| B-175 | H | H | CH₃ | CH₂CN | OCH₃ | CHF₂ |
| B-176 | H | H | CH₃ | CH₂CN | CHF₂ | OCH₃ |
| B-177 | H | H | CHF₂ | CH₂CN | CH₃ | CH₃ |
| B-178 | H | H | CHF₂ | CH₂CN | CH₃ | CHF₂ |
| B-179 | H | H | CHF₂ | CH₂CN | CHF₂ | CH₃ |
| B-180 | H | H | CHF₂ | CH₂CN | CH₃ | C≡CH |
| B-181 | H | H | CHF₂ | CH₂CN | C≡CH | CH₃ |
| B-182 | H | H | CHF₂ | CH₂CN | CH₂F | CH₃ |
| B-183 | H | H | CHF₂ | CH₂CN | CH₃ | CH₂F |
| B-184 | H | H | CHF₂ | CH₂CN | CH₂F | Br |
| B-185 | H | H | CHF₂ | CH₂CN | Br | CH₂F |
| B-186 | H | H | CHF₂ | CH₂CN | OCH₃ | CHF₂ |
| B-187 | H | H | CHF₂ | CH₂CN | CHF₂ | OCH₃ |
| B-188 | H | H | CF₃ | CH₂CN | CH₃ | CH₃ |
| B-189 | H | H | CF₃ | CH₂CN | CH₃ | CHF₂ |
| B-190 | H | H | CF₃ | CH₂CN | CHF₂ | CH₃ |
| B-191 | H | H | CF₃ | CH₂CN | CH₃ | C≡CH |
| B-192 | H | H | CF₃ | CH₂CN | C≡CH | CH₃ |
| B-193 | H | H | CF₃ | CH₂CN | CH₂F | CH₃ |
| B-194 | H | H | CF₃ | CH₂CN | CH₃ | CH₂F |
| B-195 | H | H | CF₃ | CH₂CN | CH₂F | Br |
| B-196 | H | H | CF₃ | CH₂CN | Br | CH₂F |
| B-197 | H | H | CF₃ | CH₂CN | OCH₃ | CHF₂ |
| B-198 | H | H | CF₃ | CH₂CN | CHF₂ | OCH₃ |
| B-199 | H | H | CH₃ | CH₂OCH₃ | CH₃ | CH₃ |
| B-200 | H | H | CH₃ | CH₂OCH₃ | CH₃ | CHF₂ |
| B-201 | H | H | CH₃ | CH₂OCH₃ | CHF₂ | CH₃ |
| B-202 | H | H | CH₃ | CH₂OCH₃ | CH₃ | C≡CH |
| B-203 | H | H | CH₃ | CH₂OCH₃ | C≡CH | CH₃ |
| B-204 | H | H | CH₃ | CH₂OCH₃ | CH₂F | CH₃ |
| B-205 | H | H | CH₃ | CH₂OCH₃ | CH₃ | CH₂F |
| B-206 | H | H | CH₃ | CH₂OCH₃ | CH₂F | Br |
| B-207 | H | H | CH₃ | CH₂OCH₃ | Br | CH₂F |
| B-208 | H | H | CH₃ | CH₂OCH₃ | OCH₃ | CHF₂ |
| B-209 | H | H | CH₃ | CH₂OCH₃ | CHF₂ | OCH₃ |
| B-210 | H | H | CHF₂ | CH₂OCH₃ | CH₃ | CH₃ |
| B-211 | H | H | CHF₂ | CH₂OCH₃ | CH₃ | CHF₂ |
| B-212 | H | H | CHF₂ | CH₂OCH₃ | CHF₂ | CH₃ |
| B-213 | H | H | CHF₂ | CH₂OCH₃ | CH₃ | C≡CH |
| B-214 | H | H | CHF₂ | CH₂OCH₃ | C≡CH | CH₃ |
| B-215 | H | H | CHF₂ | CH₂OCH₃ | CH₂F | CH₃ |
| B-216 | H | H | CHF₂ | CH₂OCH₃ | CH₃ | CH₂F |
| B-217 | H | H | CHF₂ | CH₂OCH₃ | CH₂F | Br |
| B-218 | H | H | CHF₂ | CH₂OCH₃ | Br | CH₂F |
| B-219 | H | H | CHF₂ | CH₂OCH₃ | OCH₃ | CHF₂ |
| B-220 | H | H | CHF₂ | CH₂OCH₃ | CHF₂ | OCH₃ |
| B-221 | H | H | CF₃ | CH₂OCH₃ | CH₃ | CH₃ |
| B-222 | H | H | CF₃ | CH₂OCH₃ | CH₃ | CHF₂ |
| B-223 | H | H | CF₃ | CH₂OCH₃ | CHF₂ | CH₃ |
| B-224 | H | H | CF₃ | CH₂OCH₃ | CH₃ | C≡CH |
| B-225 | H | H | CF₃ | CH₂OCH₃ | C≡CH | CH₃ |
| B-226 | H | H | CF₃ | CH₂OCH₃ | CH₂F | CH₃ |
| B-227 | H | H | CF₃ | CH₂OCH₃ | CH₃ | CH₂F |
| B-228 | H | H | CF₃ | CH₂OCH₃ | CH₂F | Br |
| B-229 | H | H | CF₃ | CH₂OCH₃ | Br | CH₂F |
| B-230 | H | H | CF₃ | CH₂OCH₃ | OCH₃ | CHF₂ |
| B-231 | H | H | CF₃ | CH₂OCH₃ | CHF₂ | OCH₃ |
| B-232 | H | H | CH₃ | CH₂NMe₂ | CH₃ | CH₃ |
| B-233 | H | H | CH₃ | CH₂NMe₂ | CH₃ | CHF₂ |
| B-234 | H | H | CH₃ | CH₂NMe₂ | CHF₂ | CH₃ |
| B-235 | H | H | CH₃ | CH₂NMe₂ | CH₃ | C≡CH |
| B-236 | H | H | CH₃ | CH₂NMe₂ | C≡CH | CH₃ |
| B-237 | H | H | CH₃ | CH₂NMe₂ | CH₂F | CH₃ |
| B-238 | H | H | CH₃ | CH₂NMe₂ | CH₃ | CH₂F |
| B-239 | H | H | CH₃ | CH₂NMe₂ | CH₂F | Br |
| B-240 | H | H | CH₃ | CH₂NMe₂ | Br | CH₂F |
| B-241 | H | H | CH₃ | CH₂NMe₂ | OCH₃ | CHF₂ |
| B-242 | H | H | CH₃ | CH₂NMe₂ | CHF₂ | OCH₃ |
| B-243 | H | H | CHF₂ | CH₂NMe₂ | CH₃ | CH₃ |
| B-244 | H | H | CHF₂ | CH₂NMe₂ | CH₃ | CHF₂ |
| B-245 | H | H | CHF₂ | CH₂NMe₂ | CHF₂ | CH₃ |
| B-246 | H | H | CHF₂ | CH₂NMe₂ | CH₃ | C≡CH |
| B-247 | H | H | CHF₂ | CH₂NMe₂ | C≡CH | CH₃ |
| B-248 | H | H | CHF₂ | CH₂NMe₂ | CH₂F | CH₃ |
| B-249 | H | H | CHF₂ | CH₂NMe₂ | CH₃ | CH₂F |
| B-250 | H | H | CHF₂ | CH₂NMe₂ | CH₂F | Br |
| B-251 | H | H | CHF₂ | CH₂NMe₂ | Br | CH₂F |
| B-252 | H | H | CHF₂ | CH₂NMe₂ | OCH₃ | CHF₂ |
| B-253 | H | H | CHF₂ | CH₂NMe₂ | CHF₂ | OCH₃ |
| B-254 | H | H | CF₃ | CH₂NMe₂ | CH₃ | CH₃ |
| B-255 | H | H | CF₃ | CH₂NMe₂ | CH₃ | CHF₂ |
| B-256 | H | H | CF₃ | CH₂NMe₂ | CHF₂ | CH₃ |
| B-257 | H | H | CF₃ | CH₂NMe₂ | CH₃ | C≡CH |
| B-258 | H | H | CF₃ | CH₂NMe₂ | C≡CH | CH₃ |
| B-259 | H | H | CF₃ | CH₂NMe₂ | CH₂F | CH₃ |
| B-260 | H | H | CF₃ | CH₂NMe₂ | CH₃ | CH₂F |
| B-261 | H | H | CF₃ | CH₂NMe₂ | CH₂F | Br |
| B-262 | H | H | CF₃ | CH₂NMe₂ | Br | CH₂F |
| B-263 | H | H | CF₃ | CH₂NMe₂ | OCH₃ | CHF₂ |
| B-264 | H | H | CF₃ | CH₂NMe₂ | CHF₂ | OCH₃ |
| B-265 | H | H | CH₃ | 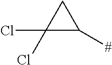 | CH₃ | CH₃ |
| B-266 | H | H | CH₃ | 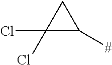 | CH₃ | CHF₂ |
| B-267 | H | H | CH₃ | 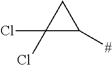 | CHF₂ | CH₃ |
| B-268 | H | H | CH₃ | 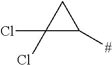 | CH₃ | C≡CH |
| B-269 | H | H | CH₃ | 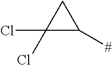 | C≡CH | CH₃ |
| B-270 | H | H | CH₃ | 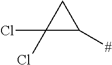 | CH₂F | CH₃ |
| B-271 | H | H | CH₃ | 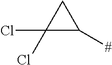 | CH₃ | CH₂F |
| B-272 | H | H | CH₃ | 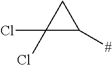 | CH₂F | Br |
| B-273 | H | H | CH₃ | 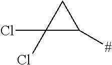 | Br | CH₂F |
| B-274 | H | H | CH₃ | 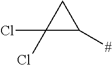 | OCH₃ | CHF₂ |
| B-275 | H | H | CH₃ | 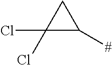 | CHF₂ | OCH₃ |
| B-276 | H | H | CHF₂ | 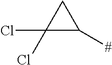 | CH₃ | CH₃ |
| B-277 | H | H | CHF₂ | 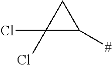 | CH₃ | CHF₂ |
| B-278 | H | H | CHF₂ | 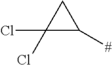 | CHF₂ | CH₃ |
| B-279 | H | H | CHF₂ | 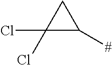 | CH₃ | C≡CH |
| B-280 | H | H | CHF₂ | 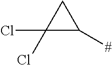 | C≡CH | CH₃ |
| B-281 | H | H | CHF₂ | 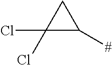 | CH₂F | CH₃ |
| B-282 | H | H | CHF₂ | 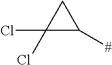 | CH₃ | CH₂F |
| B-283 | H | H | CHF₂ | 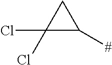 | CH₂F | Br |
| B-284 | H | H | CHF₂ | 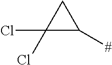 | Br | CH₂F |
| B-285 | H | H | CHF₂ | 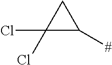 | OCH₃ | CHF₂ |
| B-286 | H | H | CHF₂ | 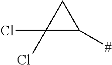 | CHF₂ | OCH₃ |
| B-287 | H | H | CF₃ | 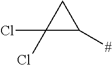 | CH₃ | CH₃ |
| B-288 | H | H | CF₃ | 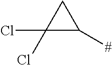 | CH₃ | CHF₂ |
| B-289 | H | H | CF₃ | 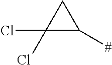 | CHF₂ | CH₃ |
| B-290 | H | H | CF₃ | 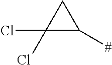 | CH₃ | C≡CH |
| B-291 | H | H | CF₃ | 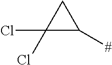 | C≡CH | CH₃ |
| B-292 | H | H | CF₃ | 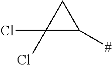 | CH₂F | CH₃ |
| B-293 | H | H | CF₃ | 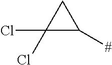 | CH₃ | CH₂F |
| B-294 | H | H | CF₃ | 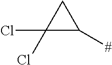 | CH₂F | Br |
| B-295 | H | H | CF₃ | 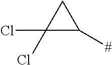 | Br | CH₂F |
| B-296 | H | H | CF₃ | 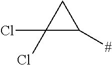 | OCH₃ | CHF₂ |
| B-297 | H | H | CF₃ | 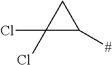 | CHF₂ | OCH₃ |
| B-298 | H | H | CH₃ | 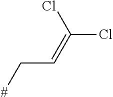 | CH₃ | CH₃ |
| B-299 | H | H | CH₃ | 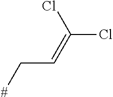 | CH₃ | CHF₂ |
| B-300 | H | H | CH₃ | 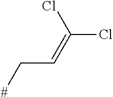 | CHF₂ | CH₃ |
| B-301 | H | H | CH₃ | 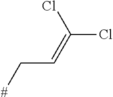 | CH₃ | C≡CH |
| B-302 | H | H | CH₃ | 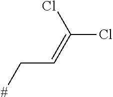 | C≡CH | CH₃ |
| B-303 | H | H | CH₃ | 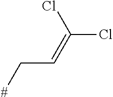 | CH₂F | CH₃ |
| B-304 | H | H | CH₃ | 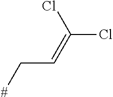 | CH₃ | CH₂F |
| B-305 | H | H | CH₃ | 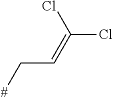 | CH₂F | Br |
| B-306 | H | H | CH₃ | 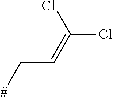 | Br | CH₂F |
| B-307 | H | H | CH₃ | 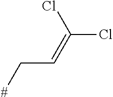 | OCH₃ | CHF₂ |
| B-308 | H | H | CH₃ | 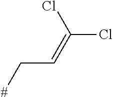 | CHF₂ | OCH₃ |
| B-309 | H | H | CHF₂ | 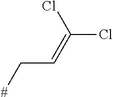 | CH₃ | CH₃ |
| B-310 | H | H | CHF₂ | 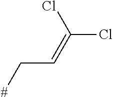 | CH₃ | CHF₂ |
| B-311 | H | H | CHF₂ | 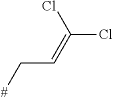 | CHF₂ | CH₃ |
| B-312 | H | H | CHF₂ | 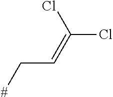 | CH₃ | C≡CH |
| B-313 | H | H | CHF₂ | 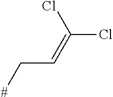 | C≡CH | CH₃ |
| B-314 | H | H | CHF₂ | 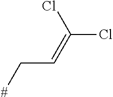 | CH₂F | CH₃ |
| B-315 | H | H | CHF₂ | 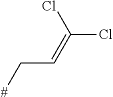 | CH₃ | CH₂F |
| B-316 | H | H | CHF₂ | 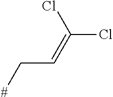 | CH₂F | Br |
| B-317 | H | H | CHF₂ | 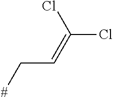 | Br | CH₂F |
| B-318 | H | H | CHF₂ | 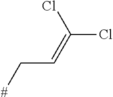 | OCH₃ | CHF₂ |
| B-319 | H | H | CHF₂ | 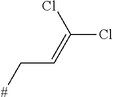 | CHF₂ | OCH₃ |
| B-320 | H | H | CF₃ | 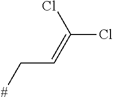 | CH₃ | CH₃ |
| B-321 | H | H | CF₃ | 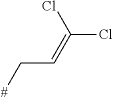 | CH₃ | CHF₂ |
| B-322 | H | H | CF₃ | 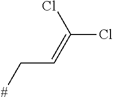 | CHF₂ | CH₃ |
| B-323 | H | H | CF₃ | 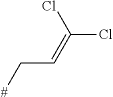 | CH₃ | C≡CH |
| B-324 | H | H | CF₃ | 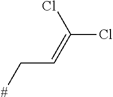 | C≡CH | CH₃ |
| B-325 | H | H | CF₃ | 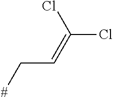 | CH₂F | CH₃ |
| B-326 | H | H | CF₃ | 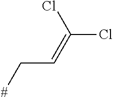 | CH₃ | CH₂F |
| B-327 | H | H | CF₃ | 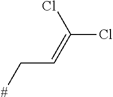 | CH₂F | Br |
| B-328 | H | H | CF₃ | 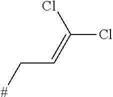 | Br | CH₂F |
| B-329 | H | H | CF₃ | 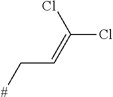 | OCH₃ | CHF₂ |
| B-330 | H | H | CF₃ | 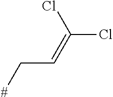 | CHF₂ | OCH₃ |
| B-331 | H | H | CH₃ | 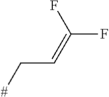 | CH₃ | CH₃ |
| B-332 | H | H | CH₃ | 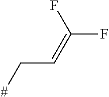 | CH₃ | CHF₂ |
| B-333 | H | H | CH₃ | 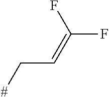 | CHF₂ | CH₃ |
| B-334 | H | H | CH₃ | 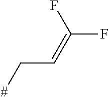 | CH₃ | C≡CH |
| B-335 | H | H | CH₃ | 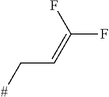 | C≡CH | CH₃ |
| B-336 | H | H | CH₃ | 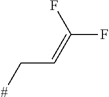 | CH₂F | CH₃ |
| B-337 | H | H | CH₃ | 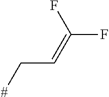 | CH₃ | CH₂F |
| B-338 | H | H | CH₃ | 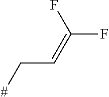 | CH₂F | Br |
| B-339 | H | H | CH₃ | 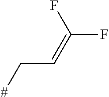 | Br | CH₂F |
| B-340 | H | H | CH₃ | 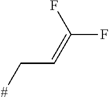 | OCH₃ | CHF₂ |
| B-341 | H | H | CH₃ | 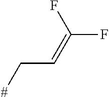 | CHF₂ | OCH₃ |
| B-342 | H | H | CHF₂ | 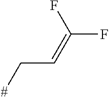 | CH₃ | CH₃ |
| B-343 | H | H | CHF₂ | 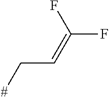 | CH₃ | CHF₂ |
| B-344 | H | H | CHF₂ | 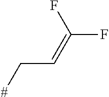 | CHF₂ | CH₃ |
| B-345 | H | H | CHF₂ | 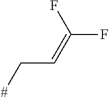 | CH₃ | C≡CH |
| B-346 | H | H | CHF₂ | 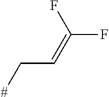 | C≡CH | CH₃ |
| B-347 | H | H | CHF₂ | 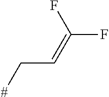 | CH₂F | CH₃ |
| B-348 | H | H | CHF₂ | 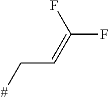 | CH₃ | CH₂F |
| B-349 | H | H | CHF₂ | 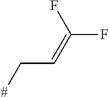 | CH₂F | Br |
| B-350 | H | H | CHF₂ | 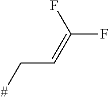 | Br | CH₂F |
| B-351 | H | H | CHF₂ | 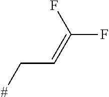 | OCH₃ | CHF₂ |
| B-352 | H | H | CHF₂ | 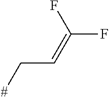 | CHF₂ | OCH₃ |
| B-353 | H | H | CF₃ | 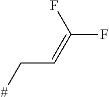 | CH₃ | CH₃ |
| B-354 | H | H | CF₃ | 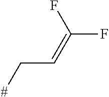 | CH₃ | CHF₂ |
| B-355 | H | H | CF₃ | 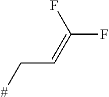 | CHF₂ | CH₃ |
| B-356 | H | H | CF₃ | 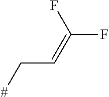 | CH₃ | C≡CH |
| B-357 | H | H | CF₃ | 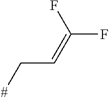 | C≡CH | CH₃ |
| B-358 | H | H | CF₃ | 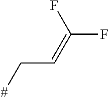 | CH₂F | CH₃ |
| B-359 | H | H | CF₃ | 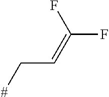 | CH₃ | CH₂F |
| B-360 | H | H | CF₃ | 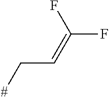 | CH₂F | Br |
| B-361 | H | H | CF₃ | 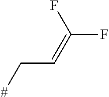 | Br | CH₂F |
| B-362 | H | H | CF₃ | 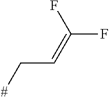 | OCH₃ | CHF₂ |
| B-363 | H | H | CF₃ | 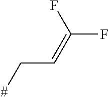 | CHF₂ | OCH₃ |
| B-364 | H | H | CH₃ | 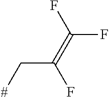 | CH₃ | CH₃ |
| B-365 | H | H | CH₃ | 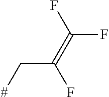 | CH₃ | CHF₂ |
| B-366 | H | H | CH₃ | 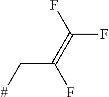 | CHF₂ | CH₃ |
| B-367 | H | H | CH₃ | 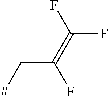 | CH₃ | C≡CH |
| B-368 | H | H | CH₃ | 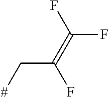 | C≡CH | CH₃ |
| B-369 | H | H | CH₃ | 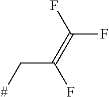 | CH₂F | CH₃ |
| B-370 | H | H | CH₃ | 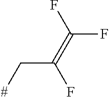 | CH₃ | CH₂F |
| B-371 | H | H | CH₃ | 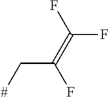 | CH₂F | Br |
| B-372 | H | H | CH₃ | 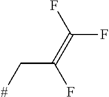 | Br | CH₂F |
| B-373 | H | H | CH₃ | 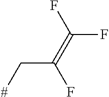 | OCH₃ | CHF₂ |
| B-374 | H | H | CH₃ | 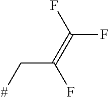 | CHF₂ | OCH₃ |
| B-375 | H | H | CHF₂ | 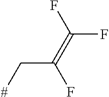 | CH₃ | CH₃ |
| B-376 | H | H | CHF₂ | 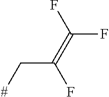 | CH₃ | CHF₂ |
| B-377 | H | H | CHF₂ | 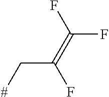 | CHF₂ | CH₃ |
| B-378 | H | H | CHF₂ | 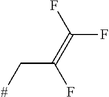 | CH₃ | C≡CH |
| B-379 | H | H | CHF₂ | 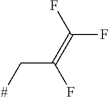 | C≡CH | CH₃ |
| B-380 | H | H | CHF₂ | 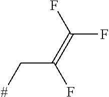 | CH₂F | CH₃ |
| B-381 | H | H | CHF₂ | 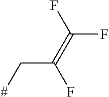 | CH₃ | CH₂F |
| B-382 | H | H | CHF₂ | 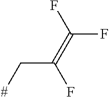 | CH₂F | Br |
| B-383 | H | H | CHF₂ | 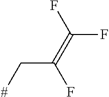 | Br | CH₂F |
| B-384 | H | H | CHF₂ | 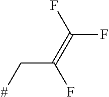 | OCH₃ | CHF₂ |
| B-385 | H | H | CHF₂ | 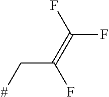 | CHF₂ | OCH₃ |
| B-386 | H | H | CF₃ | 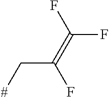 | CH₃ | CH₃ |
| B-387 | H | H | CF₃ | 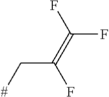 | CH₃ | CHF₂ |
| B-388 | H | H | CF₃ | 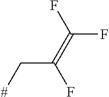 | CHF₂ | CH₃ |
| B-389 | H | H | CF₃ | 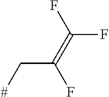 | CH₃ | C≡CH |
| B-390 | H | H | CF₃ | 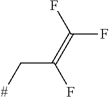 | C≡CH | CH₃ |
| B-391 | H | H | CF₃ | 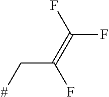 | CH₂F | CH₃ |
| B-392 | H | H | CF₃ | 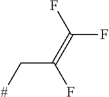 | CH₃ | CH₂F |
| B-393 | H | H | CF₃ | 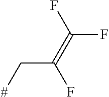 | CH₂F | Br |
| B-394 | H | H | CF₃ | 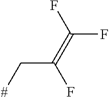 | Br | CH₂F |
| B-395 | H | H | CF₃ | 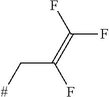 | OCH₃ | CHF₂ |
| B-396 | H | H | CF₃ | 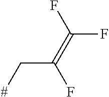 | CHF₂ | OCH₃ |
| B-397 | H | H | CH₃ | 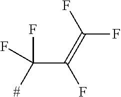 | CH₃ | CH₃ |
| B-398 | H | H | CH₃ | 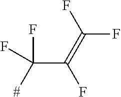 | CH₃ | CHF₂ |
| B-399 | H | H | CH₃ | 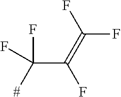 | CHF₂ | CH₃ |
| B-400 | H | H | CH₃ | 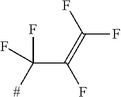 | CH₃ | C≡CH |
| B-401 | H | H | CH₃ | 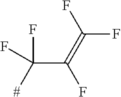 | C≡CH | CH₃ |
| B-402 | H | H | CH₃ | 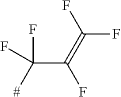 | CH₂F | CH₃ |
| B-403 | H | H | CH₃ | 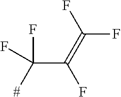 | CH₃ | CH₂F |
| B-404 | H | H | CH₃ | 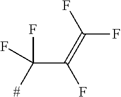 | CH₂F | Br |
| B-405 | H | H | CH₃ | 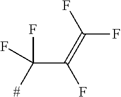 | Br | CH₂F |
| B-406 | H | H | CH₃ | 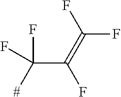 | OCH₃ | CHF₂ |
| B-407 | H | H | CH₃ | 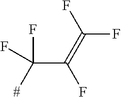 | CHF₂ | OCH₃ |
| B-408 | H | H | CHF₂ | 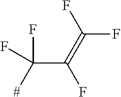 | CH₃ | CH₃ |
| B-409 | H | H | CHF₂ | 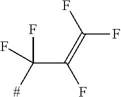 | CH₃ | CHF₂ |
| B-410 | H | H | CHF₂ | 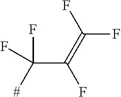 | CHF₂ | CH₃ |
| B-411 | H | H | CHF₂ | 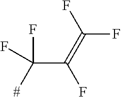 | CH₃ | C≡CH |
| B-412 | H | H | CHF₂ | 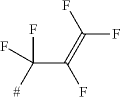 | C≡CH | CH₃ |
| B-413 | H | H | CHF₂ | 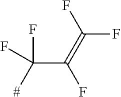 | CH₂F | CH₃ |
| B-414 | H | H | CHF₂ | 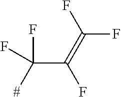 | CH₃ | CH₂F |
| B-415 | H | H | CHF₂ | 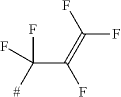 | CH₂F | Br |
| B-416 | H | H | CHF₂ | 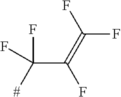 | Br | CH₂F |
| B-417 | H | H | CHF₂ | 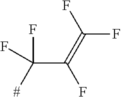 | OCH₃ | CHF₂ |
| B-418 | H | H | CHF₂ | 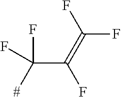 | CHF₂ | OCH₃ |
| B-419 | H | H | CF₃ | 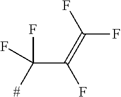 | CH₃ | CH₃ |
| B-420 | H | H | CF₃ | 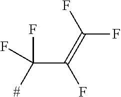 | CH₃ | CHF₂ |
| B-421 | H | H | CF₃ | 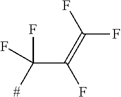 | CHF₂ | CH₃ |
| B-422 | H | H | CF₃ | 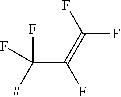 | CH₃ | C≡CH |
| B-423 | H | H | CF₃ | 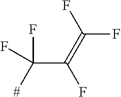 | C≡CH | CH₃ |
| B-424 | H | H | CF₃ | 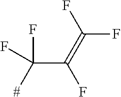 | CH₂F | CH₃ |
| B-425 | H | H | CF₃ | 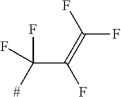 | CH₃ | CH₂F |
| B-426 | H | H | CF₃ | 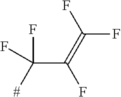 | CH₂F | Br |
| B-427 | H | H | CF₃ | 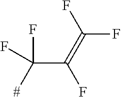 | Br | CH₂F |
| B-428 | H | H | CF₃ | 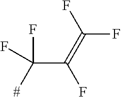 | OCH₃ | CHF₂ |
| B-429 | H | H | CF₃ | 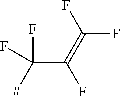 | CHF₂ | OCH₃ |
| B-430 | H | H | CH₃ | 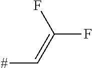 | CH₃ | CH₃ |
| B-431 | H | H | CH₃ | 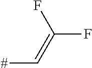 | CH₃ | CHF₂ |
| B-432 | H | H | CH₃ | 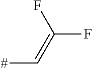 | CHF₂ | CH₃ |
| B-433 | H | H | CH₃ | 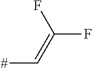 | CH₃ | C≡CH |
| B-434 | H | H | CH₃ | 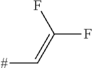 | C≡CH | CH₃ |
| B-435 | H | H | CH₃ | 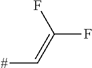 | CH₂F | CH₃ |
| B-436 | H | H | CH₃ | 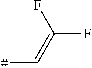 | CH₃ | CH₂F |
| B-437 | H | H | CH₃ | 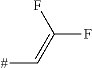 | CH₂F | Br |
| B-438 | H | H | CH₃ | 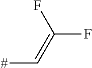 | Br | CH₂F |
| B-439 | H | H | CH₃ | 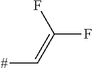 | OCH₃ | CHF₂ |
| B-440 | H | H | CH₃ | 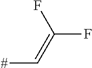 | CHF₂ | OCH₃ |
| B-441 | H | H | CHF₂ | 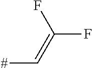 | CH₃ | CH₃ |
| B-442 | H | H | CHF₂ | 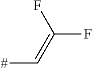 | CH₃ | CHF₂ |
| B-443 | H | H | CHF₂ | 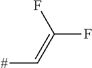 | CHF₂ | CH₃ |
| B-444 | H | H | CHF₂ | 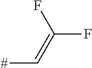 | CH₃ | C≡CH |
| B-445 | H | H | CHF₂ | 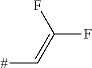 | C≡CH | CH₃ |
| B-446 | H | H | CHF₂ | 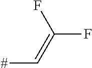 | CH₂F | CH₃ |
| B-447 | H | H | CHF₂ | 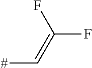 | CH₃ | CH₂F |
| B-448 | H | H | CHF₂ | 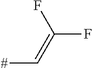 | CH₂F | Br |
| B-449 | H | H | CHF₂ | 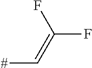 | Br | CH₂F |
| B-450 | H | H | CHF₂ | 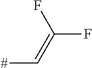 | OCH₃ | CHF₂ |
| B-451 | H | H | CHF₂ | 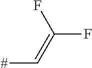 | CHF₂ | OCH₃ |
| B-452 | H | H | CF₃ | 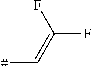 | CH₃ | CH₃ |
| B-453 | H | H | CF₃ | 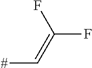 | CH₃ | CHF₂ |
| B-454 | H | H | CF₃ | 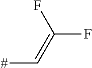 | CHF₂ | CH₃ |
| B-455 | H | H | CF₃ | 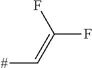 | CH₃ | C≡CH |
| B-456 | H | H | CF₃ | 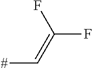 | C≡CH | CH₃ |
| B-457 | H | H | CF₃ | 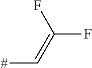 | CH₂F | CH₃ |
| B-458 | H | H | CF₃ | 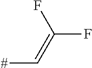 | CH₃ | CH₂F |
| B-459 | H | H | CF₃ | 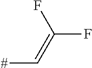 | CH₂F | Br |
| B-460 | H | H | CF₃ | 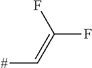 | Br | CH₂F |
| B-461 | H | H | CF₃ | 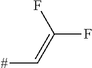 | OCH₃ | CHF₂ |
| B-462 | H | H | CF₃ | 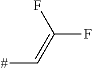 | CHF₂ | OCH₃ |
| B-463 | H | H | CH₃ | 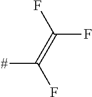 | CH₃ | CH₃ |
| B-464 | H | H | CH₃ | 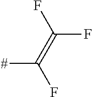 | CH₃ | CHF₂ |
| B-465 | H | H | CH₃ | 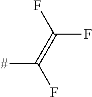 | CHF₂ | CH₃ |
| B-466 | H | H | CH₃ | 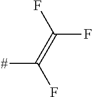 | CH₃ | C≡CH |
| B-467 | H | H | CH₃ | 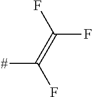 | C≡CH | CH₃ |
| B-468 | H | H | CH₃ | 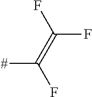 | CH₂F | CH₃ |
| B-469 | H | H | CH₃ | 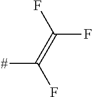 | CH₃ | CH₂F |
| B-470 | H | H | CH₃ | 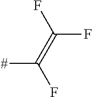 | CH₂F | Br |
| B-471 | H | H | CH₃ | 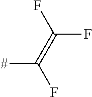 | Br | CH₂F |
| B-472 | H | H | CH₃ | 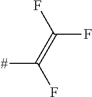 | OCH₃ | CHF₂ |
| B-473 | H | H | CH₃ | 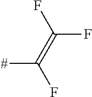 | CHF₂ | OCH₃ |
| B-474 | H | H | CHF₂ | 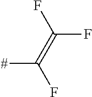 | CH₃ | CH₃ |
| B-475 | H | H | CHF₂ | 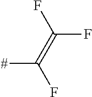 | CH₃ | CHF₂ |
| B-476 | H | H | CHF₂ | 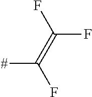 | CHF₂ | CH₃ |
| B-477 | H | H | CHF₂ | 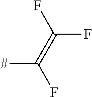 | CH₃ | C≡CH |
| B-478 | H | H | CHF₂ | 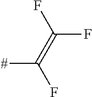 | C≡CH | CH₃ |
| B-479 | H | H | CHF₂ | 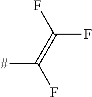 | CH₂F | CH₃ |
| B-480 | H | H | CHF₂ | 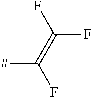 | CH₃ | CH₂F |
| B-481 | H | H | CHF₂ | 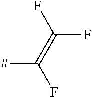 | CH₂F | Br |
| B-482 | H | H | CHF₂ | 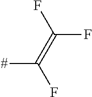 | Br | CH₂F |
| B-483 | H | H | CHF₂ | 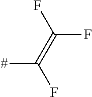 | OCH₃ | CHF₂ |
| B-484 | H | H | CHF₂ | 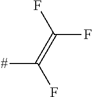 | CHF₂ | OCH₃ |
| B-485 | H | H | CF₃ | 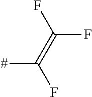 | CH₃ | CH₃ |
| B-486 | H | H | CF₃ | 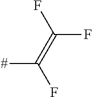 | CH₃ | CHF₂ |
| B-487 | H | H | CF₃ | 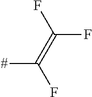 | CHF₂ | CH₃ |
| B-488 | H | H | CF₃ | 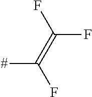 | CH₃ | C≡CH |
| B-489 | H | H | CF₃ | 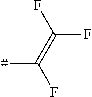 | C≡CH | CH₃ |
| B-490 | H | H | CF₃ | 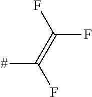 | CH₂F | CH₃ |
| B-491 | H | H | CF₃ | 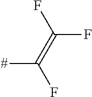 | CH₃ | CH₂F |
| B-492 | H | H | CF₃ | 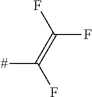 | CH₂F | Br |
| B-493 | H | H | CF₃ | 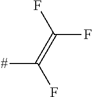 | Br | CH₂F |
| B-494 | H | H | CF₃ | 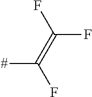 | OCH₃ | CHF₂ |
| B-495 | H | H | CF₃ | 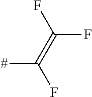 | CHF₂ | OCH₃ |
| B-496 | H | H | CH₃ | 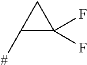 | CH₃ | CH₃ |
| B-497 | H | H | CH₃ | 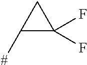 | CH₃ | CHF₂ |
| B-498 | H | H | CH₃ | 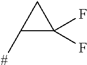 | CHF₂ | CH₃ |
| B-499 | H | H | CH₃ | 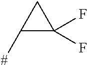 | CH₃ | C≡CH |
| B-500 | H | H | CH₃ | 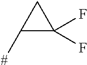 | C≡CH | CH₃ |
| B-501 | H | H | CH₃ | 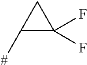 | CH₂F | CH₃ |
| B-502 | H | H | CH₃ | 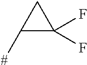 | CH₃ | CH₂F |
| B-503 | H | H | CH₃ | 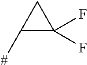 | CH₂F | Br |
| B-504 | H | H | CH₃ | 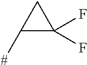 | Br | CH₂F |
| B-505 | H | H | CH₃ | 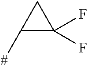 | OCH₃ | CHF₂ |
| B-506 | H | H | CH₃ | 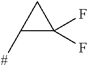 | CHF₂ | OCH₃ |
| B-507 | H | H | CHF₂ | 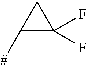 | CH₃ | CH₃ |
| B-508 | H | H | CHF₂ | 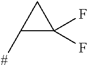 | CH₃ | CHF₂ |
| B-509 | H | H | CHF₂ | 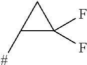 | CHF₂ | CH₃ |
| B-510 | H | H | CHF₂ | 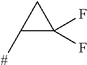 | CH₃ | C≡CH |
| B-511 | H | H | CHF₂ | 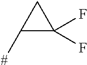 | C≡CH | CH₃ |
| B-512 | H | H | CHF₂ | 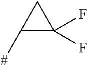 | CH₂F | CH₃ |
| B-513 | H | H | CHF₂ | 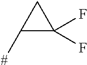 | CH₃ | CH₂F |
| B-514 | H | H | CHF₂ | 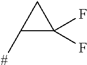 | CH₂F | Br |
| B-515 | H | H | CHF₂ | 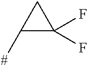 | Br | CH₂F |
| B-516 | H | H | CHF₂ | 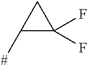 | OCH₃ | CHF₂ |
| B-517 | H | H | CHF₂ | 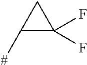 | CHF₂ | OCH₃ |
| B-518 | H | H | CF₃ | 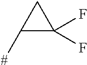 | CH₃ | CH₃ |
| B-519 | H | H | CF₃ | 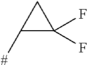 | CH₃ | CHF₂ |
| B-520 | H | H | CF₃ | 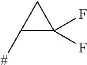 | CHF₂ | CH₃ |
| B-521 | H | H | CF₃ | 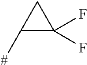 | CH₃ | C≡CH |
| B-522 | H | H | CF₃ | 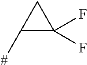 | C≡CH | CH₃ |
| B-523 | H | H | CF₃ | 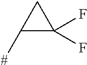 | CH₂F | CH₃ |
| B-524 | H | H | CF₃ | 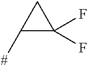 | CH₃ | CH₂F |
| B-525 | H | H | CF₃ | 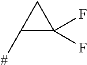 | CH₂F | Br |
| B-526 | H | H | CF₃ | 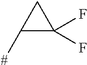 | Br | CH₂F |
| B-527 | H | H | CF₃ | 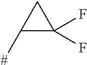 | OCH₃ | CHF₂ |
| B-528 | H | H | CF₃ | 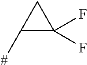 | CHF₂ | OCH₃ |
| B-529 | H | H | CH₃ | 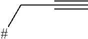 | CH₃ | CH₃ |
| B-530 | H | H | CH₃ | 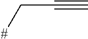 | CH₃ | CHF₂ |
| B-531 | H | H | CH₃ | 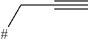 | CHF₂ | CH₃ |
| B-532 | H | H | CH₃ | 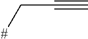 | CH₃ | C≡CH |
| B-533 | H | H | CH₃ | 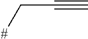 | C≡CH | CH₃ |
| B-534 | H | H | CH₃ | 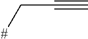 | CH₂F | CH₃ |
| B-535 | H | H | CH₃ | 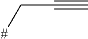 | CH₃ | CH₂F |
| B-536 | H | H | CH₃ | 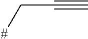 | CH₂F | Br |
| B-537 | H | H | CH₃ | 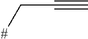 | Br | CH₂F |
| B-538 | H | H | CH₃ | 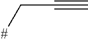 | OCH₃ | CHF₂ |
| B-539 | H | H | CH₃ | 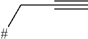 | CHF₂ | OCH₃ |
| B-540 | H | H | CHF₂ | 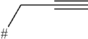 | CH₃ | CH₃ |
| B-541 | H | H | CHF₂ | 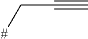 | CH₃ | CHF₂ |
| B-542 | H | H | CHF₂ | 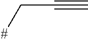 | CHF₂ | CH₃ |
| B-543 | H | H | CHF₂ | 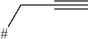 | CH₃ | C≡CH |
| B-544 | H | H | CHF₂ | 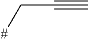 | C≡CH | CH₃ |
| B-545 | H | H | CHF₂ | 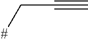 | CH₂F | CH₃ |
| B-546 | H | H | CHF₂ | 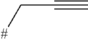 | CH₃ | CH₂F |
| B-547 | H | H | CHF₂ | 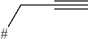 | CH₂F | Br |
| B-548 | H | H | CHF₂ | 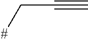 | Br | CH₂F |
| B-549 | H | H | CHF₂ | 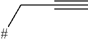 | OCH₃ | CHF₂ |
| B-550 | H | H | CHF₂ | 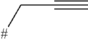 | CHF₂ | OCH₃ |
| B-551 | H | H | CF₃ | 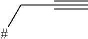 | CH₃ | CH₃ |
| B-552 | H | H | CF₃ | 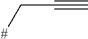 | CH₃ | CH F₂ |
| B-553 | H | H | CF₃ | 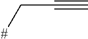 | CHF₂ | CH ₃ |
| B-554 | H | H | CF₃ | 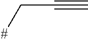 | CH₃ | C≡CH |
| B-555 | H | H | CF₃ | 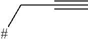 | C≡CH | CH₃ |
| B-556 | H | H | CF₃ | 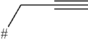 | CH₂F | CH₃ |
| B-557 | H | H | CF₃ | 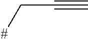 | CH₃ | CH₂F |
| B-558 | H | H | CF₃ | 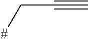 | CH₂F | Br |
| B-559 | H | H | CF₃ | 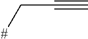 | Br | CH₂F |
| B-560 | H | H | CF₃ | 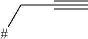 | OCH₃ | CHF₂ |
| B-561 | H | H | CF₃ | 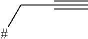 | CHF₂ | OCH₃ |
| B-562 | H | H | CH₃ | 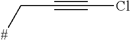 | CH₃ | CH₃ |
| B-563 | H | H | CH₃ | 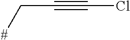 | CH₃ | CHF₂ |
| B-564 | H | H | CH₃ | 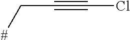 | CHF₂ | CH₃ |
| B-565 | H | H | CH₃ | 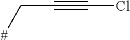 | CH₃ | C≡CH |
| B-566 | H | H | CH₃ | 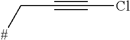 | C≡CH | CH₃ |
| B-567 | H | H | CH₃ | 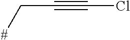 | CH₂F | CH₃ |
| B-568 | H | H | CH₃ | 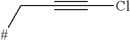 | CH₃ | CH₂F |
| B-569 | H | H | CH₃ | 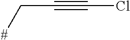 | CH₂F | Br |
| B-570 | H | H | CH₃ | 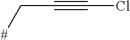 | Br | CH₂F |
| B-571 | H | H | CH₃ | 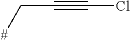 | OCH₃ | CHF₂ |
| B-572 | H | H | CH₃ | 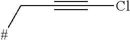 | CHF₂ | OCH₃ |
| B-573 | H | H | CHF₂ | 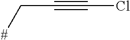 | CH₃ | CH₃ |
| B-574 | H | H | CHF₂ | 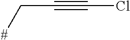 | CH₃ | CHF₂ |
| B-575 | H | H | CHF₂ | 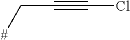 | CHF₂ | CH₃ |
| B-576 | H | H | CHF₂ | 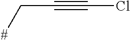 | CH₃ | C≡CH |
| B-577 | H | H | CHF₂ | 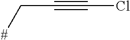 | C≡CH | CH₃ |
| B-578 | H | H | CHF₂ | 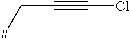 | CH₂F | CH₃ |
| B-579 | H | H | CHF₂ | 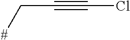 | CH₃ | CH₂F |
| B-580 | H | H | CHF₂ | 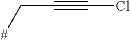 | CH₂F | Br |
| B-581 | H | H | CHF₂ | 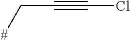 | Br | CH₂F |
| B-582 | H | H | CHF₂ | 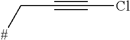 | OCH₃ | CHF₂ |
| B-583 | H | H | CHF₂ | 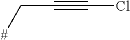 | CHF₂ | OCH₃ |
| B-584 | H | H | CF₃ | 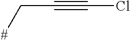 | CH₃ | CH₃ |
| B-585 | H | H | CF₃ | 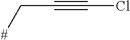 | CH₃ | CHF₂ |
| B-586 | H | H | CF₃ | 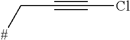 | CHF₂ | CH₃ |
| B-587 | H | H | CF₃ | 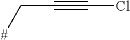 | CH₃ | C≡CH |
| B-588 | H | H | CF₃ | 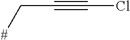 | C≡CH | CH₃ |
| B-589 | H | H | CF₃ | 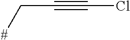 | CH₂F | CH₃ |
| B-590 | H | H | CF₃ | 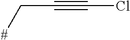 | CH₃ | CH₂F |
| B-591 | H | H | CF₃ | 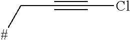 | CH₂F | Br |
| B-592 | H | H | CF₃ | 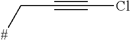 | Br | CH₂F |
| B-593 | H | H | CF₃ | 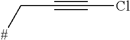 | OCH₃ | CHF₂ |
| B-594 | H | H | CF₃ | 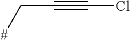 | CHF₂ | OCH₃ |
| B-595 | H | H | CH₃ | 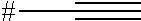 | CH₃ | CH₃ |
| B-596 | H | H | CH₃ | 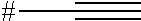 | CH₃ | CHF₂ |
| B-597 | H | H | CH₃ | 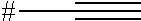 | CHF₂ | CH₃ |
| B-598 | H | H | CH₃ | 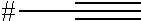 | CH₃ | C≡CH |
| B-599 | H | H | CH₃ | 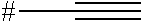 | C≡CH | CH₃ |
| B-600 | H | H | CH₃ | 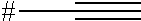 | CH₂F | CH₃ |
| B-601 | H | H | CH₃ | 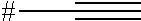 | CH₃ | CH₂F |
| B-602 | H | H | CH₃ | 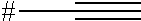 | CH₂F | Br |
| B-603 | H | H | CH₃ | 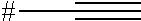 | Br | CH₂F |
| B-604 | H | H | CH₃ | 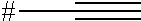 | OCH₃ | CHF₂ |
| B-605 | H | H | CH₃ | 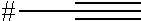 | CHF₂ | OCH₃ |
| B-606 | H | H | CHF₂ | 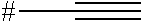 | CH₃ | CH₃ |
| B-607 | H | H | CHF₂ | 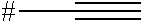 | CH₃ | CHF₂ |
| B-608 | H | H | CHF₂ | 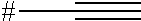 | CHF₂ | CH₃ |
| B-609 | H | H | CHF₂ | 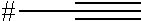 | CH₃ | C≡CH |
| B-610 | H | H | CHF₂ | 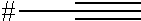 | C≡CH | CH₃ |
| B-611 | H | H | CHF₂ | 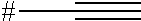 | CH₂F | CH₃ |
| B-612 | H | H | CHF₂ | 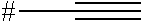 | CH₃ | CH₂F |
| B-613 | H | H | CHF₂ | 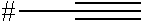 | CH₂F | Br |
| B-614 | H | H | CHF₂ | 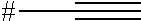 | Br | CH₂F |
| B-615 | H | H | CHF₂ | 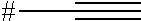 | OCH₃ | CHF₂ |
| B-616 | H | H | CHF₂ | 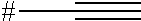 | CHF₂ | OCH₃ |
| B-617 | H | H | CF₃ | 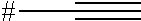 | CH₃ | CH₃ |
| B-618 | H | H | CF₃ | 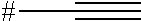 | CH₃ | CHF₂ |
| B-619 | H | H | CF₃ | 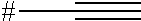 | CHF₂ | CH₃ |
| B-620 | H | H | CF₃ | 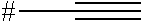 | CH₃ | C≡CH |
| B-621 | H | H | CF₃ | 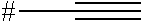 | C≡CH | CH₃ |
| B-622 | H | H | CF₃ | 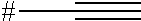 | CH₂F | CH₃ |
| B-623 | H | H | CF₃ | 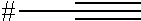 | CH₃ | CH₂F |
| B-624 | H | H | CF₃ | 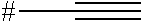 | CH₂F | Br |
| B-625 | H | H | CF₃ | 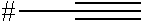 | Br | CH₂F |
| B-626 | H | H | CF₃ | 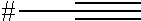 | OCH₃ | CHF₂ |
| B-627 | H | H | CF₃ | 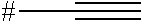 | CHF₂ | OCH₃ |
| B-628 | H | H | CH₃ | 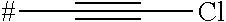 | CH₃ | CH₃ |
| B-629 | H | H | CH₃ | 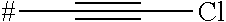 | CH₃ | CHF₂ |
| B-630 | H | H | CH₃ | 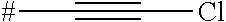 | CHF₂ | CH₃ |
| B-631 | H | H | CH₃ | 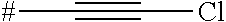 | CH₃ | C≡CH |
| B-632 | H | H | CH₃ | 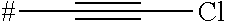 | C≡CH | CH₃ |
| B-633 | H | H | CH₃ | 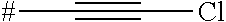 | CH₂F | CH₃ |
| B-634 | H | H | CH₃ | 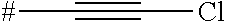 | CH₃ | CH₂F |
| B-635 | H | H | CH₃ | 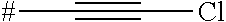 | CH₂F | Br |
| B-636 | H | H | CH₃ | 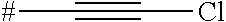 | Br | CH₂F |
| B-637 | H | H | CH₃ | 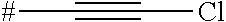 | OCH₃ | CHF₂ |
| B-638 | H | H | CH₃ | 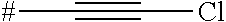 | CHF₂ | OCH₃ |
| B-639 | H | H | CHF₂ | 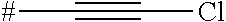 | CH₃ | CH₃ |
| B-640 | H | H | CHF₂ | 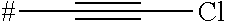 | CH₃ | CHF₂ |
| B-641 | H | H | CHF₂ | 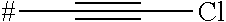 | CHF₂ | CH₃ |
| B-642 | H | H | CHF₂ | 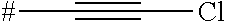 | CH₃ | C≡CH |
| B-643 | H | H | CHF₂ | 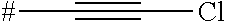 | C≡CH | CH₃ |
| B-644 | H | H | CHF₂ | 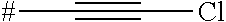 | CH₂F | CH₃ |
| B-645 | H | H | CHF₂ | 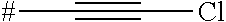 | CH₃ | CH₂F |
| B-646 | H | H | CHF₂ | 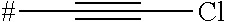 | CH₂F | Br |
| B-647 | H | H | CHF₂ | 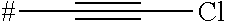 | Br | CH₂F |
| B-648 | H | H | CHF₂ | 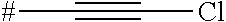 | OCH₃ | CHF₂ |
| B-649 | H | H | CHF₂ | 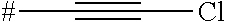 | CHF₂ | OCH₃ |
| B-650 | H | H | CF₃ | 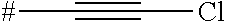 | CH₃ | CH₃ |
| B-651 | H | H | CF₃ | 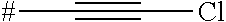 | CH₃ | CHF₂ |
| B-652 | H | H | CF₃ | 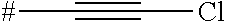 | CHF₂ | CH₃ |
| B-653 | H | H | CF₃ | 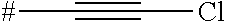 | CH₃ | C≡CH |
| B-654 | H | H | CF₃ | 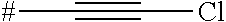 | C≡CH | CH₃ |
| B-655 | H | H | CF₃ | 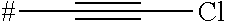 | CH₂F | CH₃ |
| B-656 | H | H | CF₃ | 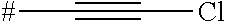 | CH₃ | CH₂F |
| B-657 | H | H | CF₃ | 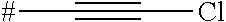 | CH₂F | Br |
| B-658 | H | H | CF₃ | 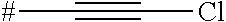 | Br | CH₂F |
| B-659 | H | H | CF₃ | 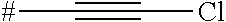 | OCH₃ | CHF₂ |
| B-660 | H | H | CF₃ | 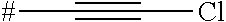 | CHF₂ | OCH₃ |
| B-661 | H | H | CH₃ | C₆H₅ | CH₃ | CH₃ |
| B-662 | H | H | CH₃ | C₆H₅ | CH₃ | CHF₂ |
| B-663 | H | H | CH₃ | C₆H₅ | CHF₂ | CH₃ |
| B-664 | H | H | CH₃ | C₆H₅ | CH₃ | C≡CH |
| B-665 | H | H | CH₃ | C₆H₅ | C≡CH | CH₃ |
| B-666 | H | H | CH₃ | C₆H₅ | CH₂F | CH₃ |
| B-667 | H | H | CH₃ | C₆H₅ | CH₃ | CH₂F |
| B-668 | H | H | CH₃ | C₆H₅ | CH₂F | Br |
| B-669 | H | H | CH₃ | C₆H₅ | Br | CH₂F |
| B-670 | H | H | CH₃ | C₆H₅ | OCH₃ | CHF₂ |
| B-671 | H | H | CH₃ | C₆H₅ | CHF₂ | OCH₃ |
| B-672 | H | H | CHF₂ | C₆H₅ | CH₃ | CH₃ |
| B-673 | H | H | CHF₂ | C₆H₅ | CH₃ | CHF₂ |
| B-674 | H | H | CHF₂ | C₆H₅ | CHF₂ | CH₃ |
| B-675 | H | H | CHF₂ | C₆H₅ | CH₃ | C≡CH |
| B-676 | H | H | CHF₂ | C₆H₅ | C≡CH | CH₃ |
| B-677 | H | H | CHF₂ | C₆H₅ | CH₂F | CH₃ |
| B-678 | H | H | CHF₂ | C₆H₅ | CH₃ | CH₂F |
| B-679 | H | H | CHF₂ | C₆H₅ | CH₂F | Br |
| B-680 | H | H | CHF₂ | C₆H₅ | Br | CH₂F |
| B-681 | H | H | CHF₂ | C₆H₅ | OCH₃ | CHF₂ |
| B-682 | H | H | CHF₂ | C₆H₅ | CHF₂ | OCH₃ |
| B-683 | H | H | CF₃ | C₆H₅ | CH₃ | CH₃ |
| B-684 | H | H | CF₃ | C₆H₅ | CH₃ | CHF₂ |
| B-685 | H | H | CF₃ | C₆H₅ | CHF₂ | CH₃ |
| B-686 | H | H | CF₃ | C₆H₅ | CH₃ | C≡CH |
| B-687 | H | H | CF₃ | C₆H₅ | C≡CH | CH₃ |
| B-688 | H | H | CF₃ | C₆H₅ | CH₂F | CH₃ |
| B-689 | H | H | CF₃ | C₆H₅ | CH₃ | CH₂F |
| B-690 | H | H | CF₃ | C₆H₅ | CH₂F | Br |
| B-691 | H | H | CF₃ | C₆H₅ | Br | CH₂F |
| B-692 | H | H | CF₃ | C₆H₅ | OCH₃ | CHF₂ |
| B-693 | H | H | CF₃ | C₆H₅ | CHF₂ | OCH₃ |
| B-694 | H | H | CH₃ | 4-Cl—C₆H₄ | CH₃ | CH₃ |
| B-695 | H | H | CH₃ | 4-Cl—C₆H₄ | CH₃ | CHF₂ |
| B-696 | H | H | CH₃ | 4-Cl—C₆H₄ | CHF₂ | CH₃ |
| B-697 | H | H | CH₃ | 4-Cl—C₆H₄ | CH₃ | C≡CH |
| B-698 | H | H | CH₃ | 4-Cl—C₆H₄ | C≡CH | CH₃ |
| B-699 | H | H | CH₃ | 4-Cl—C₆H₄ | CH₂F | CH₃ |
| B-700 | H | H | CH₃ | 4-Cl—C₆H₄ | CH₃ | CH₂F |
| B-701 | H | H | CH₃ | 4-Cl—C₆H₄ | CH₂F | Br |
| B-702 | H | H | CH₃ | 4-Cl—C₆H₄ | Br | CH₂F |
| B-703 | H | H | CH₃ | 4-Cl—C₆H₄ | OCH₃ | CHF₂ |
| B-704 | H | H | CH₃ | 4-Cl—C₆H₄ | CHF₂ | OCH₃ |
| B-705 | H | H | CHF₂ | 4-Cl—C₆H₄ | CH₃ | CH₃ |
| B-706 | H | H | CHF₂ | 4-Cl—C₆H₄ | CH₃ | CHF₂ |
| B-707 | H | H | CHF₂ | 4-Cl—C₆H₄ | CHF₂ | CH₃ |
| B-708 | H | H | CHF₂ | 4-Cl—C₆H₄ | CH₃ | C≡CH |
| B-709 | H | H | CHF₂ | 4-Cl—C₆H₄ | C≡CH | CH₃ |
| B-710 | H | H | CHF₂ | 4-Cl—C₆H₄ | CH₂F | CH₃ |
| B-711 | H | H | CHF₂ | 4-Cl—C₆H₄ | CH₃ | CH₂F |
| B-712 | H | H | CHF₂ | 4-Cl—C₆H₄ | CH₂F | Br |
| B-713 | H | H | CHF₂ | 4-Cl—C₆H₄ | Br | CH₂F |
| B-714 | H | H | CHF₂ | 4-Cl—C₆H₄ | OCH₃ | CHF₂ |
| B-715 | H | H | CHF₂ | 4-Cl—C₆H₄ | CHF₂ | OCH₃ |
| B-716 | H | H | CF₃ | 4-Cl—C₆H₄ | CH₃ | CH₃ |
| B-717 | H | H | CF₃ | 4-Cl—C₆H₄ | CH₃ | CHF₂ |
| B-718 | H | H | CF₃ | 4-Cl—C₆H₄ | CHF₂ | CH₃ |
| B-719 | H | H | CF₃ | 4-Cl—C₆H₄ | CH₃ | C≡CH |
| B-720 | H | H | CF₃ | 4-Cl—C₆H₄ | C≡CH | CH₃ |
| B-721 | H | H | CF₃ | 4-Cl—C₆H₄ | CH₂F | CH₃ |
| B-722 | H | H | CF₃ | 4-Cl—C₆H₄ | CH₃ | CH₂F |
| B-723 | H | H | CF₃ | 4-Cl—C₆H₄ | CH₂F | Br |
| B-724 | H | H | CF₃ | 4-Cl—C₆H₄ | Br | CH₂F |
| B-725 | H | H | CF₃ | 4-Cl—C₆H₄ | OCH₃ | CHF₂ |
| B-726 | H | H | CF₃ | 4-Cl—C₆H₄ | CHF₂ | OCH₃ |
| B-727 | H | H | CH₃ | 4-F—C₆H₄ | CH₃ | CH₃ |
| B-728 | H | H | CH₃ | 4-F—C₆H₄ | CH₃ | CHF₂ |
| B-729 | H | H | CH₃ | 4-F—C₆H₄ | CHF₂ | CH₃ |
| B-730 | H | H | CH₃ | 4-F—C₆H₄ | CH₃ | C≡CH |
| B-731 | H | H | CH₃ | 4-F—C₆H₄ | C≡CH | CH₃ |
| B-732 | H | H | CH₃ | 4-F—C₆H₄ | CH₂F | CH₃ |
| B-733 | H | H | CH₃ | 4-F—C₆H₄ | CH₃ | CH₂F |
| B-734 | H | H | CH₃ | 4-F—C₆H₄ | CH₂F | Br |
| B-735 | H | H | CH₃ | 4-F—C₆H₄ | Br | CH₂F |
| B-736 | H | H | CH₃ | 4-F—C₆H₄ | OCH₃ | CHF₂ |
| B-737 | H | H | CH₃ | 4-F—C₆H₄ | CHF₂ | OCH₃ |
| B-738 | H | H | CHF₂ | 4-F—C₆H₄ | CH₃ | CH₃ |
| B-739 | H | H | CHF₂ | 4-F—C₆H₄ | CH₃ | CHF₂ |
| B-740 | H | H | CHF₂ | 4-F—C₆H₄ | CHF₂ | CH₃ |
| B-741 | H | H | CHF₂ | 4-F—C₆H₄ | CH₃ | C≡CH |
| B-742 | H | H | CHF₂ | 4-F—C₆H₄ | C≡CH | CH₃ |
| B-743 | H | H | CHF₂ | 4-F—C₆H₄ | CH₂F | CH₃ |
| B-744 | H | H | CHF₂ | 4-F—C₆H₄ | CH₃ | CH₂F |
| B-745 | H | H | CHF₂ | 4-F—C₆H₄ | CH₂F | Br |
| B-746 | H | H | CHF₂ | 4-F—C₆H₄ | Br | CH₂F |
| B-747 | H | H | CHF₂ | 4-F—C₆H₄ | OCH₃ | CHF₂ |
| B-748 | H | H | CHF₂ | 4-F—C₆H₄ | CHF₂ | OCH₃ |
| B-749 | H | H | CF₃ | 4-F—C₆H₄ | CH₃ | CH₃ |
| B-750 | H | H | CF₃ | 4-F—C₆H₄ | CH₃ | CHF₂ |
| B-751 | H | H | CF₃ | 4-F—C₆H₄ | CHF₂ | CH₃ |
| B-752 | H | H | CF₃ | 4-F—C₆H₄ | CH₃ | C≡CH |
| B-753 | H | H | CF₃ | 4-F—C₆H₄ | C≡CH | CH₃ |
| B-754 | H | H | CF₃ | 4-F—C₆H₄ | CH₂F | CH₃ |
| B-755 | H | H | CF₃ | 4-F—C₆H₄ | CH₃ | CH₂F |
| B-756 | H | H | CF₃ | 4-F—C₆H₄ | CH₂F | Br |
| B-757 | H | H | CF₃ | 4-F—C₆H₄ | Br | CH₂F |
| B-758 | H | H | CF₃ | 4-F—C₆H₄ | OCH₃ | CHF₂ |
| B-759 | H | H | CF₃ | 4-F—C₆H₄ | CHF₂ | OCH₃ |
| B-760 | H | H | CH₃ | 4-CH₃O—C₆H₄ | CH₃ | CH₃ |
| B-761 | H | H | CH₃ | 4-CH₃O—C₆H₄ | CH₃ | CHF₂ |
| B-762 | H | H | CH₃ | 4-CH₃O—C₆H₄ | CHF₂ | CH₃ |
| B-763 | H | H | CH₃ | 4-CH₃O—C₆H₄ | CH₃ | C≡CH |
| B-764 | H | H | CH₃ | 4-CH₃O—C₆H₄ | C≡CH | CH₃ |
| B-765 | H | H | CH₃ | 4-CH₃O—C₆H₄ | CH₂F | CH₃ |
| B-766 | H | H | CH₃ | 4-CH₃O—C₆H₄ | CH₃ | CH₂F |
| B-767 | H | H | CH₃ | 4-CH₃O—C₆H₄ | CH₂F | Br |
| B-768 | H | H | CH₃ | 4-CH₃O—C₆H₄ | Br | CH₂F |
| B-769 | H | H | CH₃ | 4-CH₃O—C₆H₄ | OCH₃ | CHF₂ |
| B-770 | H | H | CH₃ | 4-CH₃O—C₆H₄ | CHF₂ | OCH₃ |
| B-771 | H | H | CHF₂ | 4-CH₃O—C₆H₄ | CH₃ | CH₃ |
| B-772 | H | H | CHF₂ | 4-CH₃O—C₆H₄ | CH₃ | CHF₂ |
| B-773 | H | H | CHF₂ | 4-CH₃O—C₆H₄ | CHF₂ | CH₃ |
| B-774 | H | H | CHF₂ | 4-CH₃O—C₆H₄ | CH₃ | C≡CH |
| B-775 | H | H | CHF₂ | 4-CH₃O—C₆H₄ | C≡CH | CH₃ |
| B-776 | H | H | CHF₂ | 4-CH₃O—C₆H₄ | CH₂F | CH₃ |
| B-777 | H | H | CHF₂ | 4-CH₃O—C₆H₄ | CH₃ | CH₂F |
| B-778 | H | H | CHF₂ | 4-CH₃O—C₆H₄ | CH₂F | Br |
| B-779 | H | H | CHF₂ | 4-CH₃O—C₆H₄ | Br | CH₂F |
| B-780 | H | H | CHF₂ | 4-CH₃O—C₆H₄ | OCH₃ | CHF₂ |
| B-781 | H | H | CHF₂ | 4-CH₃O—C₆H₄ | CHF₂ | OCH₃ |
| B-782 | H | H | CF₃ | 4-CH₃O—C₆H₄ | CH₃ | CH₃ |
| B-783 | H | H | CF₃ | 4-CH₃O—C₆H₄ | CH₃ | CHF₂ |
| B-784 | H | H | CF₃ | 4-CH₃O—C₆H₄ | CHF₂ | CH₃ |
| B-785 | H | H | CF₃ | 4-CH₃O—C₆H₄ | CH₃ | C≡CH |
| B-786 | H | H | CF₃ | 4-CH₃O—C₆H₄ | C≡CH | CH₃ |
| B-787 | H | H | CF₃ | 4-CH₃O—C₆H₄ | CH₂F | CH₃ |
| B-788 | H | H | CF₃ | 4-CH₃O—C₆H₄ | CH₃ | CH₂F |
| B-789 | H | H | CF₃ | 4-CH₃O—C₆H₄ | CH₂F | Br |
| B-790 | H | H | CF₃ | 4-CH₃O—C₆H₄ | Br | CH₂F |
| B-791 | H | H | CF₃ | 4-CH₃O—C₆H₄ | OCH₃ | CHF₂ |
| B-792 | H | H | CF₃ | 4-CH₃O—C₆H₄ | CHF₂ | OCH₃ |
| B-793 | H | H | CH₃ | CH₂—C₆H₅ | CH₃ | CH₃ |
| B-794 | H | H | CH₃ | CH₂—C₆H₅ | CH₃ | CHF₂ |
| B-795 | H | H | CH₃ | CH₂—C₆H₅ | CHF₂ | CH₃ |
| B-796 | H | H | CH₃ | CH₂—C₆H₅ | CH₃ | C≡CH |
| B-797 | H | H | CH₃ | CH₂—C₆H₅ | C≡CH | CH₃ |
| B-798 | H | H | CH₃ | CH₂—C₆H₅ | CH₂F | CH₃ |
| B-799 | H | H | CH₃ | CH₂—C₆H₅ | CH₃ | CH₂F |
| B-800 | H | H | CH₃ | CH₂—C₆H₅ | CH₂F | Br |
| B-801 | H | H | CH₃ | CH₂—C₆H₅ | Br | CH₂F |
| B-802 | H | H | CH₃ | CH₂—C₆H₅ | OCH₃ | CHF₂ |
| B-803 | H | H | CH₃ | CH₂—C₆H₅ | CHF₂ | OCH₃ |
| B-804 | H | H | CHF₂ | CH₂—C₆H₅ | CH₃ | CH₃ |
| B-805 | H | H | CHF₂ | CH₂—C₆H₅ | CH₃ | CHF₂ |
| B-806 | H | H | CHF₂ | CH₂—C₆H₅ | CHF₂ | CH₃ |
| B-807 | H | H | CHF₂ | CH₂—C₆H₅ | CH₃ | C≡CH |
| B-808 | H | H | CHF₂ | CH₂—C₆H₅ | C≡CH | CH₃ |
| B-809 | H | H | CHF₂ | CH₂—C₆H₅ | CH₂F | CH₃ |
| B-810 | H | H | CHF₂ | CH₂—C₆H₅ | CH₃ | CH₂F |
| B-811 | H | H | CHF₂ | CH₂—C₆H₅ | CH₂F | Br |
| B-812 | H | H | CHF₂ | CH₂—C₆H₅ | Br | CH₂F |
| B-813 | H | H | CHF₂ | CH₂—C₆H₅ | OCH₃ | CHF₂ |
| B-814 | H | H | CHF₂ | CH₂—C₆H₅ | CHF₂ | OCH₃ |
| B-815 | H | H | CF₃ | CH₂—C₆H₅ | CH₃ | CH₃ |
| B-816 | H | H | CF₃ | CH₂—C₆H₅ | CH₃ | CHF₂ |
| B-817 | H | H | CF₃ | CH₂—C₆H₅ | CHF₂ | CH₃ |
| B-818 | H | H | CF₃ | CH₂—C₆H₅ | CH₃ | C≡CH |
| B-819 | H | H | CF₃ | CH₂—C₆H₅ | C≡CH | CH₃ |
| B-820 | H | H | CF₃ | CH₂—C₆H₅ | CH₂F | CH₃ |
| B-821 | H | H | CF₃ | CH₂—C₆H₅ | CH₃ | CH₂F |
| B-822 | H | H | CF₃ | CH₂—C₆H₅ | CH₂F | Br |
| B-823 | H | H | CF₃ | CH₂—C₆H₅ | Br | CH₂F |
| B-824 | H | H | CF₃ | CH₂—C₆H₅ | OCH₃ | CHF₂ |
| B-825 | H | H | CF₃ | CH₂—C₆H₅ | CHF₂ | OCH₃ |
| B-826 | H | H | CH₃ | CH₂—C₆H₄-4-F | CH₃ | CH₃ |
| B-827 | H | H | CH₃ | CH₂—C₆H₄-4-F | CH₃ | CHF₂ |
| B-828 | H | H | CH₃ | CH₂—C₆H₄-4-F | CHF₂ | CH₃ |
| B-829 | H | H | CH₃ | CH₂—C₆H₄-4-F | CH₃ | C≡CH |
| B-830 | H | H | CH₃ | CH₂—C₆H₄-4-F | C≡CH | CH₃ |
| B-831 | H | H | CH₃ | CH₂—C₆H₄-4-F | CH₂F | CH₃ |
| B-832 | H | H | CH₃ | CH₂—C₆H₄-4-F | CH₃ | CH₂F |
| B-833 | H | H | CH₃ | CH₂—C₆H₄-4-F | CH₂F | Br |
| B-834 | H | H | CH₃ | CH₂—C₆H₄-4-F | Br | CH₂F |
| B-835 | H | H | CH₃ | CH₂—C₆H₄-4-F | OCH₃ | CHF₂ |
| B-836 | H | H | CH₃ | CH₂—C₆H₄-4-F | CHF₂ | OCH₃ |
| B-837 | H | H | CHF₂ | CH₂—C₆H₄-4-F | CH₃ | CH₃ |
| B-838 | H | H | CHF₂ | CH₂—C₆H₄-4-F | CH₃ | CHF₂ |
| B-839 | H | H | CHF₂ | CH₂—C₆H₄-4-F | CHF₂ | CH₃ |
| B-840 | H | H | CHF₂ | CH₂—C₆H₄-4-F | CH₃ | C≡CH |
| B-841 | H | H | CHF₂ | CH₂—C₆H₄-4-F | C≡CH | CH₃ |
| B-842 | H | H | CHF₂ | CH₂—C₆H₄-4-F | CH₂F | CH₃ |
| B-843 | H | H | CHF₂ | CH₂—C₆H₄-4-F | CH₃ | CH₂F |
| B-844 | H | H | CHF₂ | CH₂—C₆H₄-4-F | CH₂F | Br |
| B-845 | H | H | CHF₂ | CH₂—C₆H₄-4-F | Br | CH₂F |
| B-846 | H | H | CHF₂ | CH₂—C₆H₄-4-F | OCH₃ | CHF₂ |
| B-847 | H | H | CHF₂ | CH₂—C₆H₄-4-F | CHF₂ | OCH₃ |
| B-848 | H | H | CF₃ | CH₂—C₆H₄-4-F | CH₃ | CH₃ |
| B-849 | H | H | CF₃ | CH₂—C₆H₄-4-F | CH₃ | CHF₂ |
| B-850 | H | H | CF₃ | CH₂—C₆H₄-4-F | CHF₂ | CH₃ |
| B-851 | H | H | CF₃ | CH₂—C₆H₄-4-F | CH₃ | C≡CH |
| B-852 | H | H | CF₃ | CH₂—C₆H₄-4-F | C≡CH | CH₃ |
| B-853 | H | H | CF₃ | CH₂—C₆H₄-4-F | CH₂F | CH₃ |
| B-854 | H | H | CF₃ | CH₂—C₆H₄-4-F | CH₃ | CH₂F |
| B-855 | H | H | CF₃ | CH₂—C₆H₄-4-F | CH₂F | Br |
| B-856 | H | H | CF₃ | CH₂—C₆H₄-4-F | Br | CH₂F |
| B-857 | H | H | CF₃ | CH₂—C₆H₄-4-F | OCH₃ | CHF₂ |
| B-858 | H | H | CF₃ | CH₂—C₆H₄-4-F | CHF₂ | OCH₃ |
| B-859 | H | H | CH₃ | CH₂—C₆H₄-4-Cl | CH₃ | CH₃ |
| B-860 | H | H | CH₃ | CH₂—C₆H₄-4-Cl | CH₃ | CHF₂ |
| B-861 | H | H | CH₃ | CH₂—C₆H₄-4-Cl | CHF₂ | CH₃ |
| B-862 | H | H | CH₃ | CH₂—C₆H₄-4-Cl | CH₃ | C≡CH |
| B-863 | H | H | CH₃ | CH₂—C₆H₄-4-Cl | C≡CH | CH₃ |
| B-864 | H | H | CH₃ | CH₂—C₆H₄-4-Cl | CH₂F | CH₃ |
| B-865 | H | H | CH₃ | CH₂—C₆H₄-4-Cl | CH₃ | CH₂F |
| B-866 | H | H | CH₃ | CH₂—C₆H₄-4-Cl | CH₂F | Br |
| B-867 | H | H | CH₃ | CH₂—C₆H₄-4-Cl | Br | CH₂F |
| B-868 | H | H | CH₃ | CH₂—C₆H₄-4-Cl | OCH₃ | CHF₂ |
| B-869 | H | H | CH₃ | CH₂—C₆H₄-4-Cl | CHF₂ | OCH₃ |
| B-870 | H | H | CHF₂ | CH₂—C₆H₄-4-Cl | CH₃ | CH₃ |
| B-871 | H | H | CHF₂ | CH₂—C₆H₄-4-Cl | CH₃ | CHF₂ |
| B-872 | H | H | CHF₂ | CH₂—C₆H₄-4-Cl | CHF₂ | CH₃ |
| B-873 | H | H | CHF₂ | CH₂—C₆H₄-4-Cl | CH₃ | C≡CH |
| B-874 | H | H | CHF₂ | CH₂—C₆H₄-4-Cl | C≡CH | CH₃ |
| B-875 | H | H | CHF₂ | CH₂—C₆H₄-4-Cl | CH₂F | CH₃ |
| B-876 | H | H | CHF₂ | CH₂—C₆H₄-4-Cl | CH₃ | CH₂F |
| B-877 | H | H | CHF₂ | CH₂—C₆H₄-4-Cl | CH₂F | Br |
| B-878 | H | H | CHF₂ | CH₂—C₆H₄-4-Cl | Br | CH₂F |
| B-879 | H | H | CHF₂ | CH₂—C₆H₄-4-Cl | OCH₃ | CHF₂ |
| B-880 | H | H | CHF₂ | CH₂—C₆H₄-4-Cl | CHF₂ | OCH₃ |
| B-881 | H | H | CF₃ | CH₂—C₆H₄-4-Cl | CH₃ | CH₃ |
| B-882 | H | H | CF₃ | CH₂—C₆H₄-4-Cl | CH₃ | CHF₂ |
| B-883 | H | H | CF₃ | CH₂—C₆H₄-4-Cl | CHF₂ | CH₃ |
| B-884 | H | H | CF₃ | CH₂—C₆H₄-4-Cl | CH₃ | C≡CH |
| B-885 | H | H | CF₃ | CH₂—C₆H₄-4-Cl | C≡CH | CH₃ |
| B-886 | H | H | CF₃ | CH₂—C₆H₄-4-Cl | CH₂F | CH₃ |
| B-887 | H | H | CF₃ | CH₂—C₆H₄-4-Cl | CH₃ | CH₂F |
| B-888 | H | H | CF₃ | CH₂—C₆H₄-4-Cl | CH₂F | Br |
| B-889 | H | H | CF₃ | CH₂—C₆H₄-4-Cl | Br | CH₂F |
| B-890 | H | H | CF₃ | CH₂—C₆H₄-4-Cl | OCH₃ | CHF₂ |
| B-891 | H | H | CF₃ | CH₂—C₆H₄-4-Cl | CHF₂ | OCH₃ |
| B-892 | H | H | CH₃ | CH₂-C₆H₄-4- | CH₃ | CH₃ |
| | | | | OCH₃ | | |
| B-893 | H | H | CH₃ | CH₂-C₆H₄-4- | CH₃ | CHF₂ |
| | | | | OCH₃ | | |
| B-894 | H | H | CH₃ | CH₂-C₆H₄-4- | CHF₂ | CH₃ |
| | | | | OCH₃ | | |
| B-895 | H | H | CH₃ | CH₂-C₆H₄-4- | CH₃ | C≡CH |
| | | | | OCH₃ | | |
| B-896 | H | H | CH₃ | CH₂-C₆H₄-4- | C≡CH | CH₃ |
| | | | | OCH₃ | | |
| B-897 | H | H | CH₃ | CH₂-C₆H₄-4- | CH₂F | CH₃ |
| | | | | OCH₃ | | |
| B-898 | H | H | CH₃ | CH₂-C₆H₄-4- | CH₃ | CH₂F |
| | | | | OCH₃ | | |
| B-899 | H | H | CH₃ | CH₂-C₆H₄-4- | CH₂F | Br |
| | | | | OCH₃ | | |
| B-900 | H | H | CH₃ | CH₂-C₆H₄-4- | Br | CH₂F |
| | | | | OCH₃ | | |
| B-901 | H | H | CH₃ | CH₂-C₆H₄-4- | OCH₃ | CHF₂ |
| | | | | OCH₃ | | |
| B-902 | H | H | CH₃ | CH₂-C₆H₄-4- | CHF₂ | OCH₃ |
| | | | | OCH₃ | | |
| B-903 | H | H | CHF₂ | CH₂-C₆H₄-4- | CH₃ | CH₃ |
| | | | | OCH₃ | | |
| B-904 | H | H | CHF₂ | CH₂-C₆H₄-4- | CH₃ | CHF₂ |
| | | | | OCH₃ | | |
| B-905 | H | H | CHF₂ | CH₂-C₆H₄-4- | CHF₂ | CH₃ |
| | | | | OCH₃ | | |
| B-906 | H | H | CHF₂ | CH₂-C₆H₄-4- | CH₃ | C≡CH |
| | | | | OCH₃ | | |
| B-907 | H | H | CHF₂ | CH₂-C₆H₄-4- | C≡CH | CH₃ |
| | | | | OCH₃ | | |
| B-908 | H | H | CHF₂ | CH₂-C₆H₄-4- | CH₂F | CH₃ |
| | | | | OCH₃ | | |
| B-909 | H | H | CHF₂ | CH₂-C₆H₄-4- | CH₃ | CH₂F |
| | | | | OCH₃ | | |
| B-910 | H | H | CHF₂ | CH₂-C₆H₄-4- | CH₂F | Br |
| | | | | OCH₃ | | |
| B-911 | H | H | CHF₂ | CH₂-C₆H₄-4- | Br | CH₂F |
| | | | | OCH₃ | | |
| B-912 | H | H | CHF₂ | CH₂-C₆H₄-4- | OCH₃ | CHF₂ |
| | | | | OCH₃ | | |
| B-913 | H | H | CHF₂ | CH₂-C₆H₄-4- | CHF₂ | OCH₃ |
| | | | | OCH₃ | | |
| B-914 | H | H | CF₃ | CH₂-C₆H₄-4- | CH₃ | CH₃ |
| | | | | OCH₃ | | |
| B-915 | H | H | CF₃ | CH₂-C₆H₄-4- | CH₃ | CHF₂ |
| | | | | OCH₃ | | |
| B-916 | H | H | CF₃ | CH₂-C₆H₄-4- | CHF₂ | CH₃ |
| | | | | OCH₃ | | |
| B-917 | H | H | CF₃ | CH₂-C₆H₄-4- | CH₃ | C≡CH |
| | | | | OCH₃ | | |
| B-918 | H | H | CF₃ | CH₂-C₆H₄-4- | C≡CH | CH₃ |
| | | | | OCH₃ | | |
| B-919 | H | H | CF₃ | CH₂-C₆H₄-4- | CH₂F | CH₃ |
| | | | | OCH₃ | | |
| B-920 | H | H | CF₃ | CH₂-C₆H₄-4- | CH₃ | CH₂F |
| | | | | OCH₃ | | |
| B-921 | H | H | CF₃ | CH₂-C₆H₄-4- | CH₂F | Br |
| | | | | OCH₃ | | |
| B-922 | H | H | CF₃ | CH₂-C₆H₄-4- | Br | CH₂F |
| | | | | OCH₃ | | |
| B-923 | H | H | CF₃ | CH₂-C₆H₄-4- | OCH₃ | CHF₂ |
| | | | | OCH₃ | | |
| B-924 | H | H | CF₃ | CH₂-C₆H₄-4- | CHF₂ | OCH₃ |
| | | | | OCH₃ | | |
| B-925 | H | H | CH₃ | 2-py | CH₃ | CH₃ |
| B-926 | H | H | CH₃ | 2-py | CH₃ | CHF₂ |
| B-927 | H | H | CH₃ | 2-py | CHF₂ | CH₃ |
| B-928 | H | H | CH₃ | 2-py | CH₃ | C≡CH |
| B-929 | H | H | CH₃ | 2-py | C≡CH | CH₃ |
| B-930 | H | H | CH₃ | 2-py | CH₂F | CH₃ |
| B-931 | H | H | CH₃ | 2-py | CH₃ | CH₂F |
| B-932 | H | H | CH₃ | 2-py | CH₂F | Br |
| B-933 | H | H | CH₃ | 2-py | Br | CH₂F |
| B-934 | H | H | CH₃ | 2-py | OCH₃ | CHF₂ |
| B-935 | H | H | CH₃ | 2-py | CHF₂ | OCH₃ |
| B-936 | H | H | CHF₂ | 2-py | CH₃ | CH₃ |
| B-937 | H | H | CHF₂ | 2-py | CH₃ | CHF₂ |
| B-938 | H | H | CHF₂ | 2-py | CHF₂ | CH₃ |
| B-939 | H | H | CHF₂ | 2-py | CH₃ | C≡CH |
| B-940 | H | H | CHF₂ | 2-py | C≡CH | CH₃ |
| B-941 | H | H | CHF₂ | 2-py | CH₂F | CH₃ |
| B-942 | H | H | CHF₂ | 2-py | CH₃ | CH₂F |
| B-943 | H | H | CHF₂ | 2-py | CH₂F | Br |
| B-944 | H | H | CHF₂ | 2-py | Br | CH₂F |
| B-945 | H | H | CHF₂ | 2-py | OCH₃ | CHF₂ |
| B-946 | H | H | CHF₂ | 2-py | CHF₂ | OCH₃ |
| B-947 | H | H | CF₃ | 2-py | CH₃ | CH₃ |
| B-948 | H | H | CF₃ | 2-py | CH₃ | CHF₂ |
| B-949 | H | H | CF₃ | 2-py | CHF₂ | CH₃ |
| B-950 | H | H | CF₃ | 2-py | CH₃ | C≡CH |
| B-951 | H | H | CF₃ | 2-py | C≡CH | CH₃ |
| B-952 | H | H | CF₃ | 2-py | CH₂F | CH₃ |
| B-953 | H | H | CF₃ | 2-py | CH₃ | CH₂F |
| B-954 | H | H | CF₃ | 2-py | CH₂F | Br |
| B-955 | H | H | CF₃ | 2-py | Br | CH₂F |
| B-956 | H | H | CF₃ | 2-py | OCH₃ | CHF₂ |
| B-957 | H | H | CF₃ | 2-py | CHF₂ | OCH₃ |
| B-958 | H | H | CH₃ | 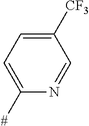 | CH₃ | CH₃ |
| B-959 | H | H | CH₃ | 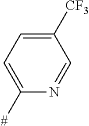 | CH₃ | CHF₂ |
| B-960 | H | H | CH₃ | 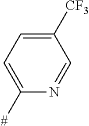 | CHF₂ | CH₃ |
| B-961 | H | H | CH₃ | 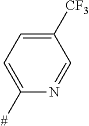 | CH₃ | C≡CH |
| B-962 | H | H | CH₃ | 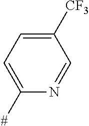 | C≡CH | CH₃ |
| B-963 | H | H | CH₃ | 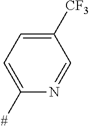 | CH₂F | CH₃ |
| B-964 | H | H | CH₃ | 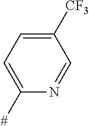 | CH₃ | CH₂F |
| B-965 | H | H | CH₃ | 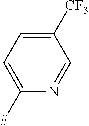 | CH₂F | Br |
| B-966 | H | H | CH₃ | 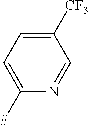 | Br | CH₂F |
| B-967 | H | H | CH₃ | 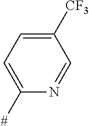 | OCH₃ | CHF₂ |
| B-968 | H | H | CH₃ | 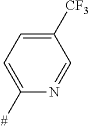 | CHF₂ | OCH₃ |
| B-969 | H | H | CHF₂ | 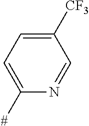 | CH₃ | CH₃ |
| B-970 | H | H | CHF₂ | 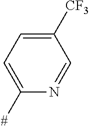 | CH₃ | CHF₂ |
| B-971 | H | H | CHF₂ | 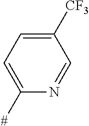 | CHF₂ | CH₃ |
| B-972 | H | H | CHF₂ | 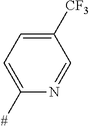 | CH₃ | C≡CH |
| B-973 | H | H | CHF₂ | 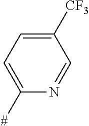 | C≡CH | CH₃ |
| B-974 | H | H | CHF₂ | 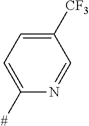 | CH₂F | CH₃ |
| B-975 | H | H | CHF₂ | 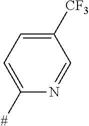 | CH₃ | CH₂F |
| B-976 | H | H | CHF₂ | 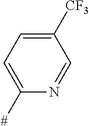 | CH₂F | Br |
| B-977 | H | H | CHF₂ | 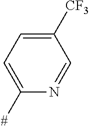 | Br | CH₂F |
| B-978 | H | H | CHF₂ | 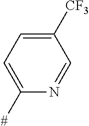 | OCH₃ | CHF₂ |
| B-979 | H | H | CHF₂ | 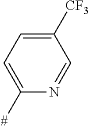 | CHF₂ | OCH₃ |
| B-980 | H | H | CF₃ | 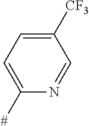 | CH₃ | CH₃ |
| B-981 | H | H | CF₃ | 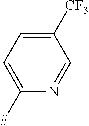 | CH₃ | CHF₂ |
| B-982 | H | H | CF₃ | 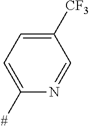 | CHF₂ | CH₃ |
| B-983 | H | H | CF₃ | 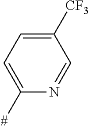 | CH₃ | C≡CH |
| B-984 | H | H | CF₃ | 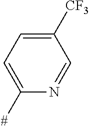 | C≡CH | CH₃ |
| B-985 | H | H | CF₃ | 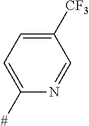 | CH₂F | CH₃ |
| B-986 | H | H | CF₃ | 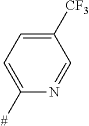 | CH₃ | CH₂F |
| B-987 | H | H | CF₃ | 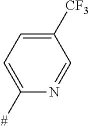 | CH₂F | Br |
| B-988 | H | H | CF₃ | 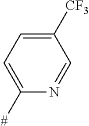 | Br | CH₂F |
| B-989 | H | H | CF₃ | 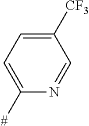 | OCH₃ | CHF₂ |
| B-990 | H | H | CF₃ | 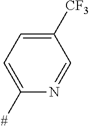 | CHF₂ | OCH₃ |
| B-991 | H | H | CH₃ | 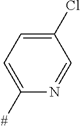 | CH₃ | CH₃ |
| B-992 | H | H | CH₃ | 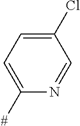 | CH₃ | CHF₂ |
| B-993 | H | H | CH₃ | 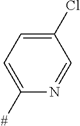 | CHF₂ | CH₃ |
| B-994 | H | H | CH₃ | 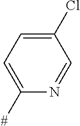 | CH₃ | C≡CH |
| B-995 | H | H | CH₃ | 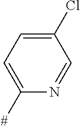 | C≡CH | CH₃ |
| B-996 | H | H | CH₃ | 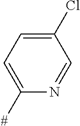 | CH₂F | CH₃ |
| B-997 | H | H | CH₃ | 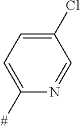 | CH₃ | CH₂F |
| B-998 | H | H | CH₃ | 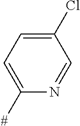 | CH₂F | Br |
| B-999 | H | H | CH₃ | 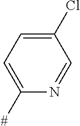 | Br | CH₂F |
| B-1000 | H | H | CH₃ | 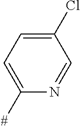 | OCH₃ | CHF₂ |
| B-1001 | H | H | CH₃ | 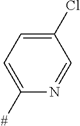 | CHF₂ | OCH₃ |
| B-1002 | H | H | CHF₂ | 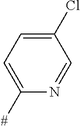 | CH₃ | CH₃ |
| B-1003 | H | H | CHF₂ | 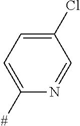 | CH₃ | CHF₂ |
| B-1004 | H | H | CHF₂ | 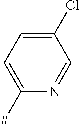 | CHF₂ | CH₃ |
| B-1005 | H | H | CHF₂ | 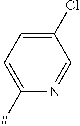 | CH₃ | C≡CH |
| B-1006 | H | H | CHF₂ | 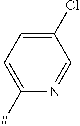 | C≡CH | CH₃ |
| B-1007 | H | H | CHF₂ | 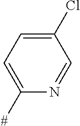 | CH₂F | CH₃ |
| B-1008 | H | H | CHF₂ | 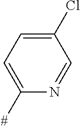 | CH₃ | CH₂F |
| B-1009 | H | H | CHF₂ | 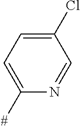 | CH₂F | Br |
| B-1010 | H | H | CHF₂ | 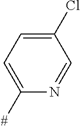 | Br | CH₂F |
| B-1011 | H | H | CHF₂ | 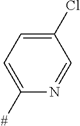 | OCH₃ | CHF₂ |
| B-1012 | H | H | CHF₂ | 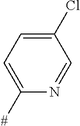 | CHF₂ | OCH₃ |
| B-1013 | H | H | CF₃ | 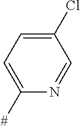 | CH₃ | CH₃ |
| B-1014 | H | H | CF₃ | 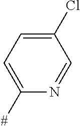 | CH₃ | CHF₂ |
| B-1015 | H | H | CF₃ | 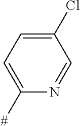 | CHF₂ | CH₃ |
| B-1016 | H | H | CF₃ | 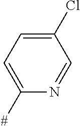 | CH₃ | C≡CH |
| B-1017 | H | H | CF₃ | 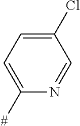 | C≡CH | CH₃ |
| B-1018 | H | H | CF₃ | 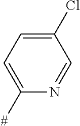 | CH₂F | CH₃ |
| B-1019 | H | H | CF₃ | 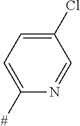 | CH₃ | CH₂F |
| B-1020 | H | H | CF₃ | 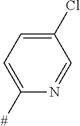 | CH₂F | Br |
| B-1021 | H | H | CF₃ | 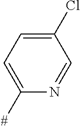 | Br | CH₂F |
| B-1022 | H | H | CF₃ | 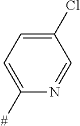 | OCH₃ | CHF₂ |
| B-1023 | H | H | CF₃ | 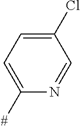 | CHF₂ | OCH₃ |
| B-1024 | H | H | CH₃ | 3-py | CH₃ | CH₃ |
| B-1025 | H | H | CH₃ | 3-py | CH₃ | CHF₂ |
| B-1026 | H | H | CH₃ | 3-py | CHF₂ | CH₃ |
| B-1027 | H | H | CH₃ | 3-py | CH₃ | C≡CH |
| B-1028 | H | H | CH₃ | 3-py | C≡CH | CH₃ |
| B-1029 | H | H | CH₃ | 3-py | CH₂F | CH₃ |
| B-1030 | H | H | CH₃ | 3-py | CH₃ | CH₂F |
| B-1031 | H | H | CH₃ | 3-py | CH₂F | Br |
| B-1032 | H | H | CH₃ | 3-py | Br | CH₂F |
| B-1033 | H | H | CH₃ | 3-py | OCH₃ | CHF₂ |
| B-1034 | H | H | CH₃ | 3-py | CHF₂ | OCH₃ |
| B-1035 | H | H | CHF₂ | 3-py | CH₃ | CH₃ |
| B-1036 | H | H | CHF₂ | 3-py | CH₃ | CHF₂ |
| B-1037 | H | H | CHF₂ | 3-py | CHF₂ | CH₃ |
| B-1038 | H | H | CHF₂ | 3-py | CH₃ | C≡CH |
| B-1039 | H | H | CHF₂ | 3-py | C≡CH | CH₃ |
| B-1040 | H | H | CHF₂ | 3-py | CH₂F | CH₃ |
| B-1041 | H | H | CHF₂ | 3-py | CH₃ | CH₂F |
| B-1042 | H | H | CHF₂ | 3-py | CH₂F | Br |
| B-1043 | H | H | CHF₂ | 3-py | Br | CH₂F |
| B-1044 | H | H | CHF₂ | 3-py | OCH₃ | CHF₂ |
| B-1045 | H | H | CHF₂ | 3-py | CHF₂ | OCH₃ |
| B-1046 | H | H | CF₃ | 3-py | CH₃ | CH₃ |
| B-1047 | H | H | CF₃ | 3-py | CH₃ | CHF₂ |
| B-1048 | H | H | CF₃ | 3-py | CHF₂ | CH₃ |
| B-1049 | H | H | CF₃ | 3-py | CH₃ | C≡CH |
| B-1050 | H | H | CF₃ | 3-py | C≡CH | CH₃ |
| B-1051 | H | H | CF₃ | 3-py | CH₂F | CH₃ |
| B-1052 | H | H | CF₃ | 3-py | CH₃ | CH₂F |
| B-1053 | H | H | CF₃ | 3-py | CH₂F | Br |
| B-1054 | H | H | CF₃ | 3-py | Br | CH₂F |
| B-1055 | H | H | CF₃ | 3-py | OCH₃ | CHF₂ |
| B-1056 | H | H | CF₃ | 3-py | CHF₂ | OCH₃ |
| B-1057 | H | H | CH₃ | 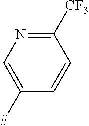 | CH₃ | CH₃ |
| B-1058 | H | H | CH₃ | 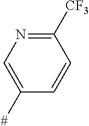 | CH₃ | CHF₂ |
| B-1059 | H | H | CH₃ | 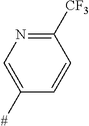 | CHF₂ | CH₃ |
| B-1060 | H | H | CH₃ | 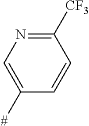 | CH₃ | C≡CH |
| B-1061 | H | H | CH₃ | 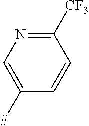 | C≡CH | CH₃ |
| B-1062 | H | H | CH₃ | 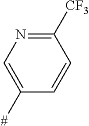 | CH₂F | CH₃ |
| B-1063 | H | H | CH₃ | 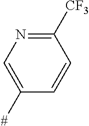 | CH₃ | CH₂F |
| B-1064 | H | H | CH₃ | 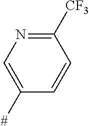 | CH₂F | Br |
| B-1065 | H | H | CH₃ | 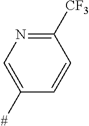 | Br | CH₂F |
| B-1066 | H | H | CH₃ | 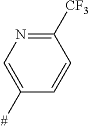 | OCH₃ | CHF₂ |
| B-1067 | H | H | CH₃ | 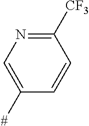 | CHF₂ | OCH₃ |
| B-1068 | H | H | CHF₂ | 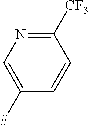 | CH₃ | CH₃ |
| B-1069 | H | H | CHF₂ | 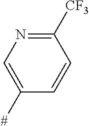 | CH₃ | CHF₂ |
| B-1070 | H | H | CHF₂ | 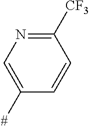 | CHF₂ | CH₃ |
| B-1071 | H | H | CHF₂ | 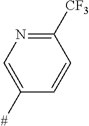 | CH₃ | C≡CH |
| B-1072 | H | H | CHF₂ | 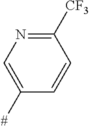 | C≡CH | CH₃ |
| B-1073 | H | H | CHF₂ | 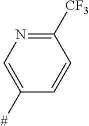 | CH₂F | CH₃ |
| B-1074 | H | H | CHF₂ | 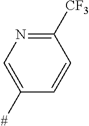 | CH₃ | CH₂F |
| B-1075 | H | H | CHF₂ | 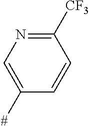 | CH₂F | Br |
| B-1076 | H | H | CHF₂ | 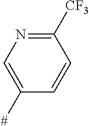 | Br | CH₂F |
| B-1077 | H | H | CHF₂ | 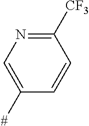 | OCH₃ | CHF₂ |
| B-1078 | H | H | CHF₂ | 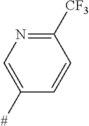 | CHF₂ | OCH₃ |
| B-1079 | H | H | CF₃ | 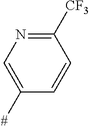 | CH₃ | CH₃ |
| B-1080 | H | H | CF₃ | 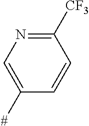 | CH₃ | CHF₂ |
| B-1081 | H | H | CF₃ | 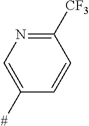 | CHF₂ | CH₃ |
| B-1082 | H | H | CF₃ | 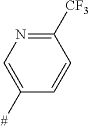 | CH₃ | C≡CH |
| B-1083 | H | H | CF₃ | 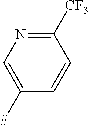 | C≡CH | CH₃ |
| B-1084 | H | H | CF₃ | 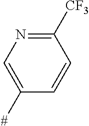 | CH₂F | CH₃ |
| B-1085 | H | H | CF₃ | 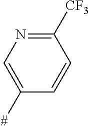 | CH₃ | CH₂F |
| B-1086 | H | H | CF₃ | 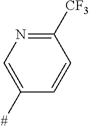 | CH₂F | Br |
| B-1087 | H | H | CF₃ | 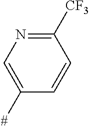 | Br | CH₂F |
| B-1088 | H | H | CF₃ | 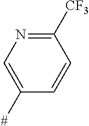 | OCH₃ | CHF₂ |
| B-1089 | H | H | CF₃ | 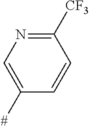 | CHF₂ | OCH₃ |
| B-1090 | H | H | CH₃ | 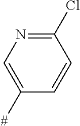 | CH₃ | CH₃ |
| B-1091 | H | H | CH₃ | 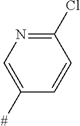 | CH₃ | CHF₂ |
| B-1092 | H | H | CH₃ | 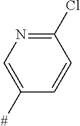 | CHF₂ | CH₃ |
| B-1093 | H | H | CH₃ | 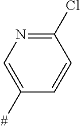 | CH₃ | C≡CH |
| B-1094 | H | H | CH₃ | 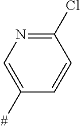 | C≡CH | CH₃ |
| B-1095 | H | H | CH₃ | 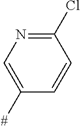 | CH₂F | CH₃ |
| B-1096 | H | H | CH₃ | 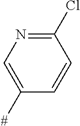 | CH₃ | CH₂F |
| B-1097 | H | H | CH₃ | 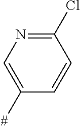 | CH₂F | Br |
| B-1098 | H | H | CH₃ | 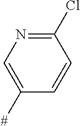 | Br | CH₂F |
| B-1099 | H | H | CH₃ | 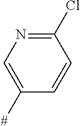 | OCH₃ | CHF₂ |
| B-1100 | H | H | CH₃ | 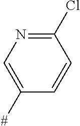 | CHF₂ | OCH₃ |
| B-1101 | H | H | CHF₂ | 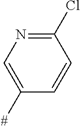 | CH₃ | CH₃ |
| B-1102 | H | H | CHF₂ | 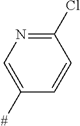 | CH₃ | CHF₂ |
| B-1103 | H | H | CHF₂ | 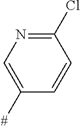 | CHF₂ | CH₃ |
| B-1104 | H | H | CHF₂ | 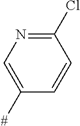 | CH₃ | C≡CH |
| B-1105 | H | H | CHF₂ | 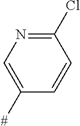 | C≡CH | CH₃ |
| B-1106 | H | H | CHF₂ | 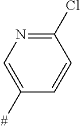 | CH₂F | CH₃ |
| B-1107 | H | H | CHF₂ | 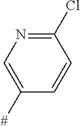 | CH₃ | CH₂F |
| B-1108 | H | H | CHF₂ | 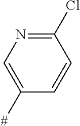 | CH₂F | Br |
| B-1109 | H | H | CHF₂ | 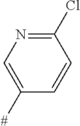 | Br | CH₂F |
| B-1110 | H | H | CHF₂ | 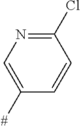 | OCH₃ | CHF₂ |
| B-1111 | H | H | CHF₂ | 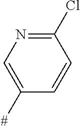 | CHF₂ | OCH₃ |
| B-1112 | H | H | CF₃ | 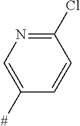 | CH₃ | CH₃ |
| B-1113 | H | H | CF₃ | 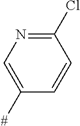 | CH₃ | CHF₂ |
| B-1114 | H | H | CF₃ | 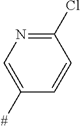 | CHF₂ | CH₃ |
| B-1115 | H | H | CF₃ | 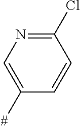 | CH₃ | C≡CH |
| B-1116 | H | H | CF₃ | 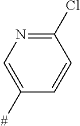 | C≡CH | CH₃ |
| B-1117 | H | H | CF₃ | 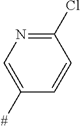 | CH₂F | CH₃ |
| B-1118 | H | H | CF₃ | 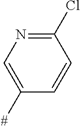 | CH₃ | CH₂F |
| B-1119 | H | H | CF₃ | 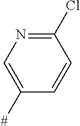 | CH₂F | Br |
| B-1120 | H | H | CF₃ | 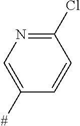 | Br | CH₂F |
| B-1121 | H | H | CF₃ | 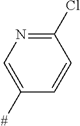 | OCH₃ | CHF₂ |
| B-1122 | H | H | CF₃ | 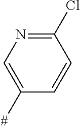 | CHF₂ | OCH₃ |
| B-1123 | H | H | CH₃ | ₄-py | CH₃ | CH₃ |
| B-1124 | H | H | CH₃ | ₄-py | CH₃ | CHF₂ |
| B-1125 | H | H | CH₃ | ₄-py | CHF₂ | CH₃ |
| B-1126 | H | H | CH₃ | ₄-py | CH₃ | C≡CH |
| B-1127 | H | H | CH₃ | ₄-py | C≡CH | CH₃ |
| B-1128 | H | H | CH₃ | ₄-py | CH₂F | CH₃ |
| B-1129 | H | H | CH₃ | ₄-py | CH₃ | CH₂F |
| B-1130 | H | H | CH₃ | ₄-py | CH₂F | Br |
| B-1131 | H | H | CH₃ | ₄-py | Br | CH₂F |
| B-1132 | H | H | CH₃ | 4-py | OCH₃ | CHF₂ |
| B-1133 | H | H | CH₃ | 4-py | CHF₂ | OCH₃ |
| B-1134 | H | H | CHF₂ | 4-py | CH₃ | CH₃ |
| B-1135 | H | H | CHF₂ | 4-py | CH₃ | CHF₂ |
| B-1136 | H | H | CHF₂ | 4-py | CHF₂ | CH₃ |
| B-1137 | H | H | CHF₂ | 4-py | CH₃ | C≡CH |
| B-1138 | H | H | CHF₂ | 4-py | C≡CH | CH₃ |
| B-1139 | H | H | CHF₂ | 4-py | CH₂F | CH₃ |
| B-1140 | H | H | CHF₂ | 4-py | CH₃ | CH₂F |
| B-1141 | H | H | CHF₂ | 4-py | CH₂F | Br |
| B-1142 | H | H | CHF₂ | 4-py | Br | CH₂F |
| B-1143 | H | H | CHF₂ | 4-py | OCH₃ | CHF₂ |
| B-1144 | H | H | CHF₂ | 4-py | CHF₂ | OCH₃ |
| B-1145 | H | H | CF₃ | 4-py | CH₃ | CH₃ |
| B-1146 | H | H | CF₃ | 4-py | CH₃ | CHF₂ |
| B-1147 | H | H | CF₃ | 4-py | CHF₂ | CH₃ |
| B-1148 | H | H | CF₃ | 4-py | CH₃ | C≡CH |
| B-1149 | H | H | CF₃ | 4-py | C≡CH | CH₃ |
| B-1150 | H | H | CF₃ | 4-py | CH₂F | CH₃ |
| B-1151 | H | H | CF₃ | 4-py | CH₃ | CH₂F |
| B-1152 | H | H | CF₃ | 4-py | CH₂F | Br |
| B-1153 | H | H | CF₃ | 4-py | Br | CH₂F |
| B-1154 | H | H | CF₃ | 4-py | OCH₃ | CHF₂ |
| B-1155 | H | H | CF₃ | 4-py | CHF₂ | OCH₃ |
| B-1156 | H | H | CH₃ | 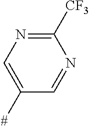 | CH₃ | CH₃ |
| B-1157 | H | H | CH₃ | 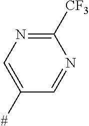 | CH₃ | CHF₂ |
| B-1158 | H | H | CH₃ | 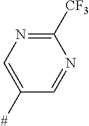 | CHF₂ | CH₃ |
| B-1159 | H | H | CH₃ | 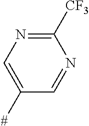 | CH₃ | C≡CH |
| B-1160 | H | H | CH₃ | 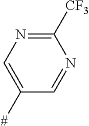 | C≡CH | CH₃ |
| B-1161 | H | H | CH₃ | 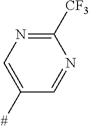 | CH₂F | CH₃ |
| B-1162 | H | H | CH₃ | 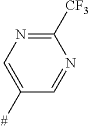 | CH₃ | CH₂F |
| B-1163 | H | H | CH₃ | 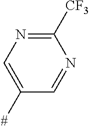 | CH₂F | Br |
| B-1164 | H | H | CH₃ | 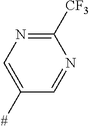 | Br | CH₂F |
| B-1165 | H | H | CH₃ | 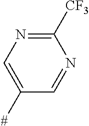 | OCH₃ | CHF₂ |
| B-1166 | H | H | CH₃ | 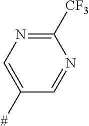 | CHF₂ | OCH₃ |
| B-1167 | H | H | CHF₂ | 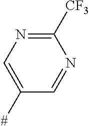 | CH₃ | CH₃ |
| B-1168 | H | H | CHF₂ | 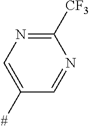 | CH₃ | CHF₂ |
| B-1169 | H | H | CHF₂ | 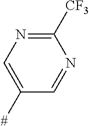 | CHF₂ | CH₃ |
| B-1170 | H | H | CHF₂ | 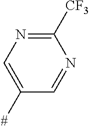 | CH₃ | C≡CH |
| B-1171 | H | H | CHF₂ | 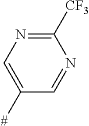 | C≡CH | CH₃ |
| B-1172 | H | H | CHF₂ | 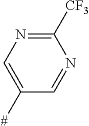 | CH₂F | CH₃ |
| B-1173 | H | H | CHF₂ | 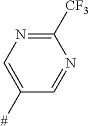 | CH₃ | CH₂F |
| B-1174 | H | H | CHF₂ | 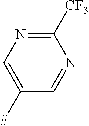 | CH₂F | Br |
| B-1175 | H | H | CHF₂ | 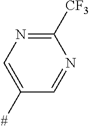 | Br | CH₂F |
| B-1176 | H | H | CHF₂ | 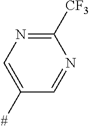 | OCH₃ | CHF₂ |
| B-1177 | H | H | CHF₂ | 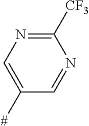 | CHF₂ | OCH₃ |
| B-1178 | H | H | CF₃ | 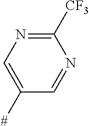 | CH₃ | CH₃ |
| B-1179 | H | H | CF₃ | 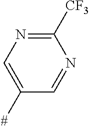 | CH₃ | CHF₂ |
| B-1180 | H | H | CF₃ | 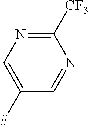 | CHF₂ | CH₃ |
| B-1181 | H | H | CF₃ | 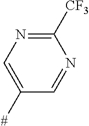 | CH₃ | C≡CH |
| B-1182 | H | H | CF₃ | 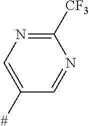 | C≡CH | CH₃ |
| B-1183 | H | H | CF₃ | 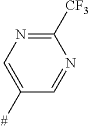 | CH₂F | CH₃ |
| B-1184 | H | H | CF₃ | 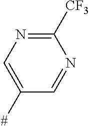 | CH₃ | CH₂F |
| B-1185 | H | H | CF₃ | 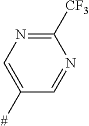 | CH₂F | Br |
| B-1186 | H | H | CF₃ | 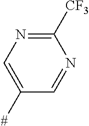 | Br | CH₂F |
| B-1187 | H | H | CF₃ | 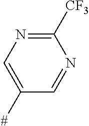 | OCH₃ | CHF₂ |
| B-1188 | H | H | CF₃ | 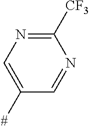 | CHF₂ | OCH₃ |
| B-1189 | H | H | CH₃ | 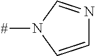 | CH₃ | CH₃ |
| B-1190 | H | H | CH₃ | 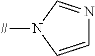 | CH₃ | CHF₂ |
| B-1191 | H | H | CH₃ | 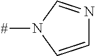 | CHF₂ | CH₃ |
| B-1192 | H | H | CH₃ | 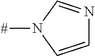 | CH₃ | C≡CH |
| B-1193 | H | H | CH₃ | 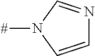 | C≡CH | CH₃ |
| B-1194 | H | H | CH₃ | 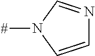 | CH₂F | CH₃ |
| B-1195 | H | H | CH₃ | 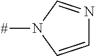 | CH₃ | CH₂F |
| B-1196 | H | H | CH₃ | 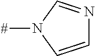 | CH₂F | Br |
| B-1197 | H | H | CH₃ | 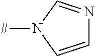 | Br | CH₂F |
| B-1198 | H | H | CH₃ | 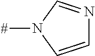 | OCH₃ | CHF₂ |
| B-1199 | H | H | CH₃ | 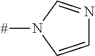 | CHF₂ | OCH₃ |
| B-1200 | H | H | CHF₂ | 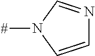 | CH₃ | CH₃ |
| B-1201 | H | H | CHF₂ | 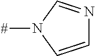 | CH₃ | CHF₂ |
| B-1202 | H | H | CHF₂ | 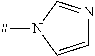 | CHF₂ | CH₃ |
| B-1203 | H | H | CHF₂ | 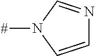 | CH₃ | C≡CH |
| B-1204 | H | H | CHF₂ | 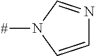 | C≡CH | CH₃ |
| B-1205 | H | H | CHF₂ | 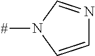 | CH₂F | CH₃ |
| B-1206 | H | H | CHF₂ | 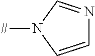 | CH₃ | CH₂F |
| B-1207 | H | H | CHF₂ | 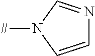 | CH₂F | Br |
| B-1208 | H | H | CHF₂ | 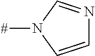 | Br | CH₂F |
| B-1209 | H | H | CHF₂ | 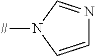 | OCH₃ | CHF₂ |
| B-1210 | H | H | CHF₂ | 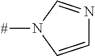 | CHF₂ | OCH₃ |
| B-1211 | H | H | CF₃ | 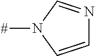 | CH₃ | CH₃ |
| B-1212 | H | H | CF₃ | 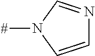 | CH₃ | CHF₂ |
| B-1213 | H | H | CF₃ | 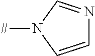 | CHF₂ | CH₃ |
| B-1214 | H | H | CF₃ | 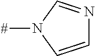 | CH₃ | C≡CH |
| B-1215 | H | H | CF₃ | 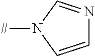 | C≡CH | CH₃ |
| B-1216 | H | H | CF₃ | 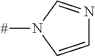 | CH₂F | CH₃ |
| B-1217 | H | H | CF₃ | 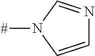 | CH₃ | CH₂F |
| B-1218 | H | H | CF₃ | 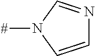 | CH₂F | Br |
| B-1219 | H | H | CF₃ | 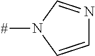 | Br | CH₂F |
| B-1220 | H | H | CF₃ | 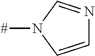 | OCH₃ | CHF₂ |
| B-1221 | H | H | CF₃ | 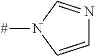 | CHF₂ | OCH₃ |
| B-1222 | H | H | CH₃ | 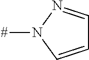 | CH₃ | CH₃ |
| B-1223 | H | H | CH₃ | 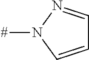 | CH₃ | CHF₂ |
| B-1224 | H | H | CH₃ | 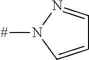 | CHF₂ | CH₃ |
| B-1225 | H | H | CH₃ | 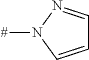 | CH₃ | C≡CH |
| B-1226 | H | H | CH₃ | 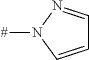 | C≡CH | CH₃ |
| B-1227 | H | H | CH₃ | 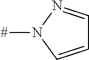 | CH₂F | CH₃ |
| B-1228 | H | H | CH₃ | 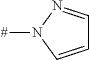 | CH₃ | CH₂F |
| B-1229 | H | H | CH₃ | 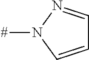 | CH₂F | Br |
| B-1230 | H | H | CH₃ | 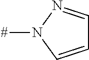 | Br | CH₂F |
| B-1231 | H | H | CH₃ | 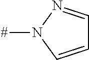 | OCH₃ | CHF₂ |
| B-1232 | H | H | CH₃ | 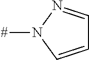 | CHF₂ | OCH₃ |
| B-1233 | H | H | CHF₂ | 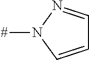 | CH₃ | CH₃ |
| B-1234 | H | H | CHF₂ | 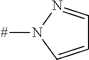 | CH₃ | CH F₂ |
| B-1235 | H | H | CHF₂ | 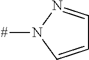 | CHF₂ | CH₃ |
| B-1236 | H | H | CHF₂ | 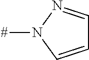 | CH₃ | C≡CH |
| B-1237 | H | H | CHF₂ | 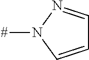 | C≡CH | CH₃ |
| B-1238 | H | H | CHF₂ | 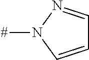 | CH₂F | CH₃ |
| B-1239 | H | H | CHF₂ | 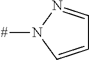 | CH₃ | CH₂F |
| B-1240 | H | H | CHF₂ | 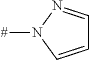 | CH₂F | Br |
| B-1241 | H | H | CHF₂ | 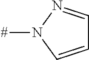 | Br | CH₂F |
| B-1242 | H | H | CHF₂ | 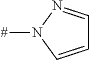 | OCH₃ | CHF₂ |
| B-1243 | H | H | CHF₂ | 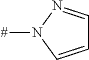 | CHF₂ | OCH₃ |
| B-1244 | H | H | CF₃ | 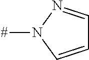 | CH₃ | CH₃ |
| B-1245 | H | H | CF₃ | 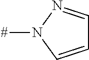 | CH₃ | CHF₂ |
| B-1246 | H | H | CF₃ | 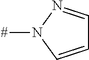 | CHF₂ | CH₃ |
| B-1247 | H | H | CF₃ | 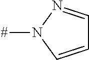 | CH₃ | C≡CH |
| B-1248 | H | H | CF₃ | 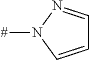 | C≡CH | CH₃ |
| B-1249 | H | H | CF₃ | 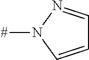 | CH₂F | CH₃ |
| B-1250 | H | H | CF₃ | 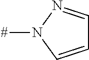 | CH₃ | CH₂F |
| B-1251 | H | H | CF₃ | 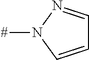 | CH₂F | Br |
| B-1252 | H | H | CF₃ | 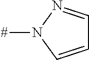 | Br | CH₂F |
| B-1253 | H | H | CF₃ | 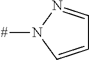 | OCH₃ | CHF₂ |
| B-1254 | H | H | CF₃ | 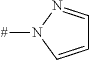 | CHF₂ | OCH₃ |
| B-1255 | H | H | CH₃ | 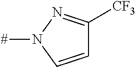 | CH₃ | CH₃ |
| B-1256 | H | H | CH₃ | 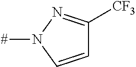 | CH₃ | CHF₂ |
| B-1257 | H | H | CH₃ | 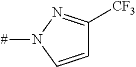 | CHF₂ | CH₃ |
| B-1258 | H | H | CH₃ | 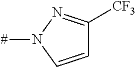 | CH₃ | C≡CH |
| B-1259 | H | H | CH₃ | 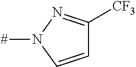 | C≡CH | CH₃ |
| B-1260 | H | H | CH₃ | 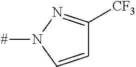 | CH₂F | CH₃ |
| B-1261 | H | H | CH₃ | 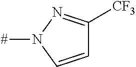 | CH₃ | CH₂F |
| B-1262 | H | H | CH₃ | 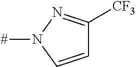 | CH₂F | Br |
| B-1263 | H | H | CH₃ | 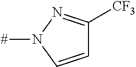 | Br | CH₂F |
| B-1264 | H | H | CH₃ | 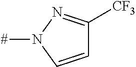 | OCH₃ | CHF₂ |
| B-1265 | H | H | CH₃ | 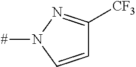 | CHF₂ | OCH₃ |
| B-1266 | H | H | CHF₂ | 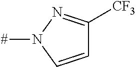 | CH₃ | CH₃ |
| B-1267 | H | H | CHF₂ | 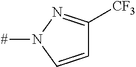 | CH₃ | CHF₂ |
| B-1268 | H | H | CHF₂ | 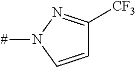 | CHF₂ | CH₃ |
| B-1269 | H | H | CHF₂ | 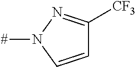 | CH₃ | C≡CH |
| B-1270 | H | H | CHF₂ | 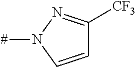 | C≡CH | CH₃ |
| B-1271 | H | H | CHF₂ | 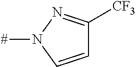 | CH₂F | CH₃ |
| B-1272 | H | H | CHF₂ | 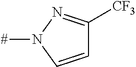 | CH₃ | CH₂F |
| B-1273 | H | H | CHF₂ | 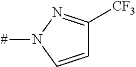 | CH₂F | Br |
| B-1274 | H | H | CHF₂ | 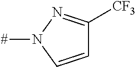 | Br | CH₂F |
| B-1275 | H | H | CHF₂ | 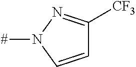 | OCH₃ | CHF₂ |
| B-1276 | H | H | CHF₂ | 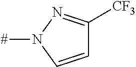 | CHF₂ | OCH₃ |
| B-1277 | H | H | CF₃ | 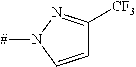 | CH₃ | CH₃ |
| B-1278 | H | H | CF₃ | 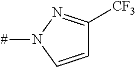 | CH₃ | CHF₂ |
| B-1279 | H | H | CF₃ | 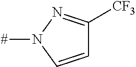 | CHF₂ | CH₃ |
| B-1280 | H | H | CF₃ | 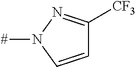 | CH₃ | C≡CH |
| B-1281 | H | H | CF₃ | 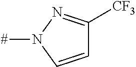 | C≡CH | CH₃ |
| B-1282 | H | H | CF₃ | 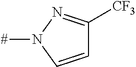 | CH₂F | CH₃ |
| B-1283 | H | H | CF₃ | 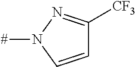 | CH₃ | CH₂F |
| B-1284 | H | H | CF₃ | 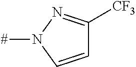 | CH₂F | Br |
| B-1285 | H | H | CF₃ | 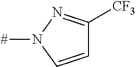 | Br | CH₂F |
| B-1286 | H | H | CF₃ | 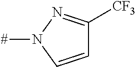 | OCH₃ | CHF₂ |
| B-1287 | H | H | CF₃ | 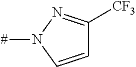 | CHF₂ | OCH₃ |
| B-1288 | H | H | CH₃ | 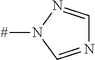 | CH₃ | CH₃ |
| B-1289 | H | H | CH₃ | 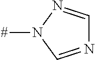 | CH₃ | CHF₂ |
| B-1290 | H | H | CH₃ | 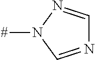 | CHF₂ | CH₃ |
| B-1291 | H | H | CH₃ | 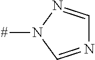 | CH₃ | C≡CH |
| B-1292 | H | H | CH₃ | 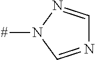 | C≡CH | CH₃ |
| B-1293 | H | H | CH₃ | 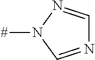 | CH₂F | CH₃ |
| B-1294 | H | H | CH₃ | 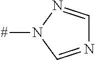 | CH₃ | CH₂F |
| B-1295 | H | H | CH₃ | 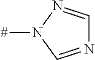 | CH₂F | Br |
| B-1296 | H | H | CH₃ | 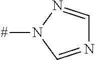 | Br | CH₂F |
| B-1297 | H | H | CH₃ | 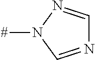 | OCH₃ | CHF₂ |
| B-1298 | H | H | CH₃ | 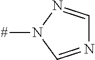 | CHF₂ | OCH₃ |
| B-1299 | H | H | CHF₂ | 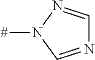 | CH₃ | CH₃ |
| B-1300 | H | H | CHF₂ | 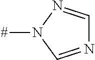 | CH₃ | CHF₂ |
| B-1301 | H | H | CHF₂ | 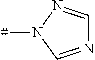 | CHF₂ | CH₃ |
| B-1302 | H | H | CHF₂ | 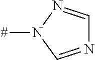 | CH₃ | C≡CH |
| B-1303 | H | H | CHF₂ | 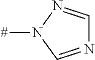 | C≡CH | CH₃ |
| B-1304 | H | H | CHF₂ | 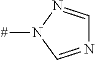 | CH₂F | CH₃ |
| B-1305 | H | H | CHF₂ | 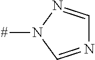 | CH₃ | CH₂F |
| B-1306 | H | H | CHF₂ | 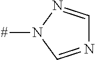 | CH₂F | Br |
| B-1307 | H | H | CHF₂ | 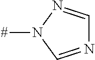 | Br | CH₂F |
| B-1308 | H | H | CHF₂ | 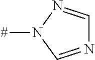 | OCH₃ | CHF₂ |
| B-1309 | H | H | CHF₂ | 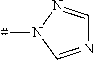 | CHF₂ | OCH₃ |
| B-1310 | H | H | CF₃ | 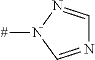 | CH₃ | CH₃ |
| B-1311 | H | H | CF₃ | 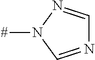 | CH₃ | CHF₂ |
| B-1312 | H | H | CF₃ | 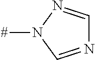 | CHF₂ | CH₃ |
| B-1313 | H | H | CF₃ | 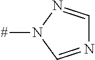 | CH₃ | C≡CH |
| B-1314 | H | H | CF₃ | 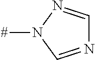 | C≡CH | CH₃ |
| B-1315 | H | H | CF₃ | 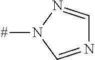 | CH₂F | CH₃ |
| B-1316 | H | H | CF₃ | 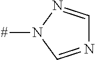 | CH₃ | CH₂F |
| B-1317 | H | H | CF₃ | 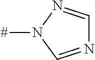 | CH₂F | Br |
| B-1318 | H | H | CF₃ | 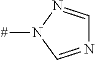 | Br | CH₂F |
| B-1319 | H | H | CF₃ | 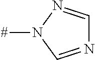 | OCH₃ | CHF₂ |
| B-1320 | H | H | CF₃ | 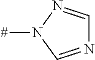 | CHF₂ | OC≡1₃ |
| B-1321 | H | H | CH₃ | 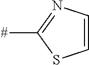 | CH₃ | CH₃ |
| B-1322 | H | H | CH₃ | 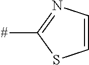 | CH₃ | CHF₂ |
| B-1323 | H | H | CH₃ | 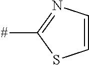 | CHF₂ | CH₃ |
| B-1324 | H | H | CH₃ | 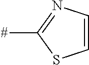 | CH₃ | C≡CH |
| B-1325 | H | H | CH₃ | 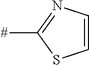 | C≡CH | CH₃ |
| B-1326 | H | H | CH₃ | 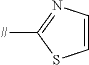 | CH₂F | CH₃ |
| B-1327 | H | H | CH₃ | 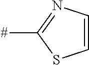 | CH₃ | CH₂F |
| B-1328 | H | H | CH₃ | 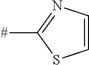 | CH₂F | Br |
| B-1329 | H | H | CH₃ | 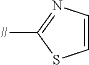 | Br | CH₂F |
| B-1330 | H | H | CH₃ | 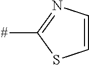 | OCH₃ | CHF₂ |
| B-1331 | H | H | CH₃ | 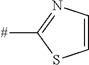 | CHF₂ | OCH₃ |
| B-1332 | H | H | CHF₂ | 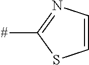 | CH₃ | CH₃ |
| B-1333 | H | H | CHF₂ | 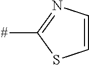 | CH₃ | CHF₂ |
| B-1334 | H | H | CHF₂ | 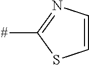 | CHF₂ | CH₃ |
| B-1335 | H | H | CHF₂ | 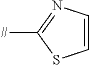 | CH₃ | C≡CH |
| B-1336 | H | H | CHF₂ | 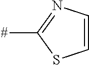 | C≡CH | CH₃ |
| B-1337 | H | H | CHF₂ | 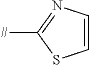 | CH₂F | CH₃ |
| B-1338 | H | H | CHF₂ | 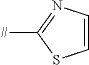 | CH₃ | CH₂F |
| B-1339 | H | H | CHF₂ | 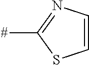 | CH₂F | Br |
| B-1340 | H | H | CHF₂ | 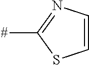 | Br | CH₂F |
| B-1341 | H | H | CHF₂ | 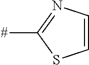 | OCH₃ | CHF₂ |
| B-1342 | H | H | CHF₂ | 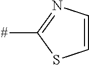 | CHF₂ | OCH₃ |
| B-1343 | H | H | CF₃ | 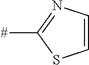 | CH₃ | CH₃ |
| B-1344 | H | H | CF₃ | 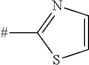 | CH₃ | CHF₂ |
| B-1345 | H | H | CF₃ | 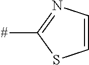 | CHF₂ | CH₃ |
| B-1346 | H | H | CF₃ | 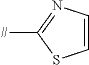 | CH₃ | C≡CH |
| B-1347 | H | H | CF₃ | 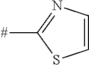 | C≡CH | CH₃ |
| B-1348 | H | H | CF₃ | 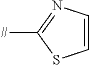 | CH₂F | CH₃ |
| B-1349 | H | H | CF₃ | 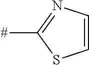 | CH₃ | CH₂F |
| B-1350 | H | H | CF₃ | 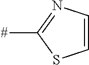 | CH₂F | Br |
| B-1351 | H | H | CF₃ | 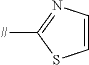 | Br | CH₂F |
| B-1352 | H | H | CF₃ | 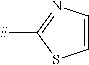 | OCH₃ | CHF₂ |
| B-1353 | H | H | CF₃ | 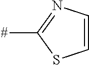 | CHF₂ | OCH₃ |
| B-1354 | H | H | CH₃ | 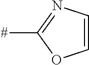 | CH₃ | CH₃ |
| B-1355 | H | H | CH₃ | 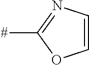 | CH₃ | CHF₂ |
| B-1356 | H | H | CH₃ | 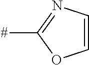 | CHF₂ | CH₃ |
| B-1357 | H | H | CH₃ | 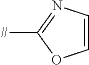 | CH₃ | C≡CH |
| B-1358 | H | H | CH₃ | 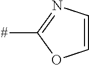 | C≡CH | CH₃ |
| B-1359 | H | H | CH₃ | 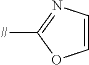 | CH₂F | CH₃ |
| B-1360 | H | H | CH₃ | 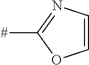 | CH₃ | CH₂F |
| B-1361 | H | H | CH₃ | 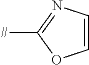 | CH₂F | Br |
| B-1362 | H | H | CH₃ | 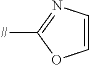 | Br | CH₂F |
| B-1363 | H | H | CH₃ | 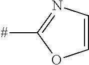 | OCH₃ | CHF₂ |
| B-1364 | H | H | CH₃ | 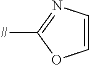 | CHF₂ | OCH₃ |
| B-1365 | H | H | CHF₂ | 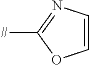 | CH₃ | CH₃ |
| B-1366 | H | H | CHF₂ | 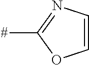 | CH₃ | CHF₂ |
| B-1367 | H | H | CHF₂ | 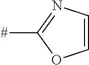 | CHF₂ | CH₃ |
| B-1368 | H | H | CHF₂ | 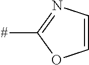 | CH₃ | C≡CH |
| B-1369 | H | H | CHF₂ | 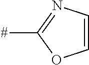 | C≡CH | CH₃ |
| B-1370 | H | H | CHF₂ | 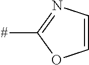 | CH₂F | CH₃ |
| B-1371 | H | H | CHF₂ | 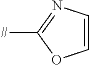 | CH₃ | CH₂F |
| B-1372 | H | H | CHF₂ | 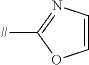 | CH₂F | Br |
| B-1373 | H | H | CHF₂ | 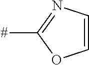 | Br | CH₂F |
| B-1374 | H | H | CHF₂ | 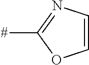 | OCH₃ | CHF₂ |
| B-1375 | H | H | CHF₂ | 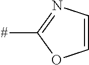 | CHF₂ | OCH₃ |
| B-1376 | H | H | CF₃ | 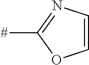 | CH₃ | CH₃ |
| B-1377 | H | H | CF₃ | 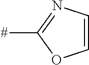 | CH₃ | CHF₂ |
| B-1378 | H | H | CF₃ | 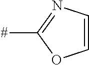 | CHF₂ | CH₃ |
| B-1379 | H | H | CF₃ | 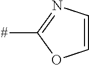 | CH₃ | C≡CH |
| B-1380 | H | H | CF₃ | 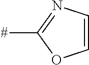 | C≡CH | CH₃ |
| B-1381 | H | H | CF₃ | 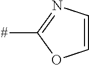 | CH₂F | CH₃ |
| B-1382 | H | H | CF₃ | 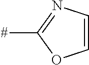 | CH₃ | CH₂F |
| B-1383 | H | H | CF₃ | 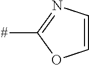 | CH₂F | Br |
| B-1384 | H | H | CF₃ | 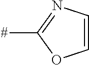 | Br | CH₂F |
| B-1385 | H | H | CF₃ | 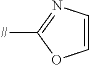 | OCH₃ | CHF₂ |
| B-1386 | H | H | CF₃ | 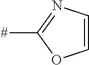 | CHF₂ | OCH₃ |
| B-1387 | H | H | CH₃ | 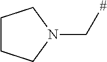 | CH₃ | CH₃ |
| B-1388 | H | H | CH₃ | 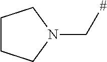 | CH₃ | CHF₂ |
| B-1389 | H | H | CH₃ | 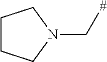 | CHF₂ | CH₃ |
| B-1390 | H | H | CH₃ | 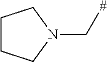 | CH₃ | C≡CH |
| B-1391 | H | H | CH₃ | 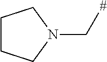 | C≡CH | CH₃ |
| B-1392 | H | H | CH₃ | 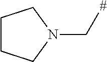 | CH₂F | CH₃ |
| B-1393 | H | H | CH₃ | 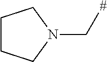 | CH₃ | CH₂F |
| B-1394 | H | H | CH₃ | 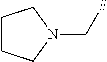 | CH₂F | Br |
| B-1395 | H | H | CH₃ | 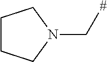 | Br | CH₂F |
| B-1396 | H | H | CH₃ | 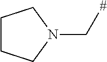 | OCH₃ | CHF₂ |
| B-1397 | H | H | CH₃ | 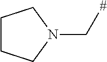 | CHF₂ | OCH₃ |
| B-1398 | H | H | CHF₂ | 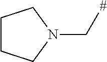 | CH₃ | CH₃ |
| B-1399 | H | H | CHF₂ | 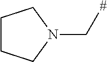 | CH₃ | CHF₂ |
| B-1400 | H | H | CHF₂ | 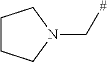 | CHF₂ | CH₃ |
| B-1401 | H | H | CHF₂ | 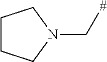 | CH₃ | C≡CH |
| B-1402 | H | H | CHF₂ | 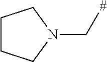 | C≡CH | CH₃ |
| B-1403 | H | H | CHF₂ | 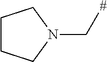 | CH₂F | CH₃ |
| B-1404 | H | H | CHF₂ | 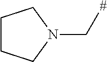 | CH₃ | CH₂F |
| B-1405 | H | H | CHF₂ | 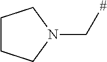 | CH₂F | Br |
| B-1406 | H | H | CHF₂ | 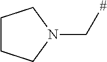 | Br | CH₂F |
| B-1407 | H | H | CHF₂ | 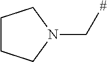 | OCH₃ | CHF₂ |
| B-1408 | H | H | CHF₂ | 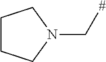 | CHF₂ | OCH₃ |
| B-1409 | H | H | CF₃ | 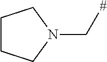 | CH₃ | CH₃ |
| B-1410 | H | H | CF₃ | 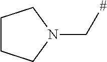 | CH₃ | CHF₂ |
| B-1411 | H | H | CF₃ | 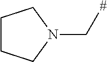 | CHF₂ | CH₃ |
| B-1412 | H | H | CF₃ | 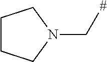 | CH₃ | C≡CH |
| B-1413 | H | H | CF₃ | 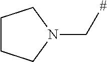 | C≡CH | CH₃ |
| B-1414 | H | H | CF₃ | 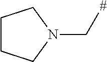 | CH₂F | CH₃ |
| B-1415 | H | H | CF₃ | 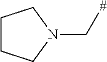 | CH₃ | CH₂F |
| B-1416 | H | H | CF₃ | 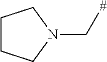 | CH₂F | Br |
| B-1417 | H | H | CF₃ | 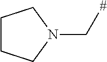 | Br | CH₂F |
| B-1418 | H | H | CF₃ | 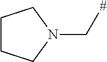 | OCH₃ | CHF₂ |
| B-1419 | H | H | CF₃ | 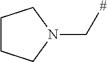 | CHF₂ | OCH₃ |
| B-1420 | H | H | CH₃ | 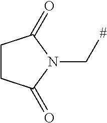 | CH₃ | CH₃ |
| B-1421 | H | H | CH₃ | 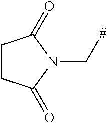 | CH₃ | CHF₂ |
| B-1422 | H | H | CH₃ | 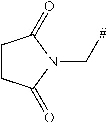 | CHF₂ | CH₃ |
| B-1423 | H | H | CH₃ | 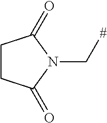 | CH₃ | C≡CH |
| B-1424 | H | H | CH₃ | 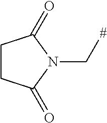 | C≡CH | CH₃ |
| B-1425 | H | H | CH₃ | 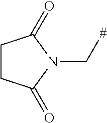 | CH₂F | CH₃ |
| B-1426 | H | H | CH₃ | 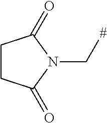 | CH₃ | CH₂F |
| B-1427 | H | H | CH₃ | 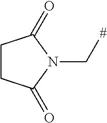 | CH₂F | Br |
| B-1428 | H | H | CH₃ | 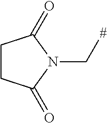 | Br | CH₂F |
| B-1429 | H | H | CH₃ | 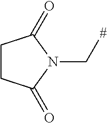 | OCH₃ | CHF₂ |
| B-1430 | H | H | CH₃ | 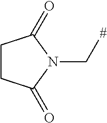 | CHF₂ | OCH₃ |
| B-1431 | H | H | CHF₂ | 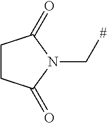 | CH₃ | CH₃ |
| B-1432 | H | H | CHF₂ | 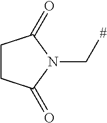 | CH₃ | CHF₂ |
| B-1433 | H | H | CHF₂ | 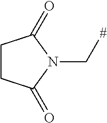 | CHF₂ | CH₃ |
| B-1434 | H | H | CHF₂ | 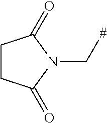 | CH₃ | C≡CH |
| B-1435 | H | H | CHF₂ | 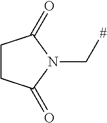 | C≡CH | CH₃ |
| B-1436 | H | H | CHF₂ | 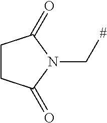 | CH₂F | CH₃ |
| B-1437 | H | H | CHF₂ | 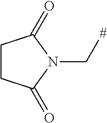 | CH₃ | CH₂F |
| B-1438 | H | H | CHF₂ | 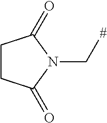 | CH₂F | Br |
| B-1439 | H | H | CHF₂ | 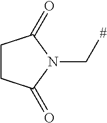 | Br | CH₂F |
| B-1440 | H | H | CHF₂ | 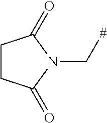 | OCH₃ | CHF₂ |
| B-1441 | H | H | CHF₂ | 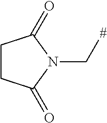 | CHF₂ | OCH₃ |
| B-1442 | H | H | CF₃ | 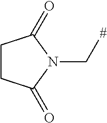 | CH₃ | CH₃ |
| B-1443 | H | H | CF₃ | 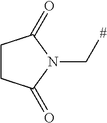 | CH₃ | CHF₂ |
| B-1444 | H | H | CF₃ | 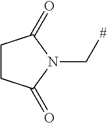 | CHF₂ | CH₃ |
| B-1445 | H | H | CF₃ | 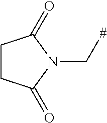 | CH₃ | C≡CH |
| B-1446 | H | H | CF₃ | 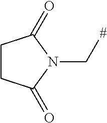 | C≡CH | CH₃ |
| B-1447 | H | H | CF₃ | 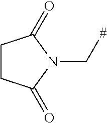 | CH₂F | CH₃ |
| B-1448 | H | H | CF₃ | 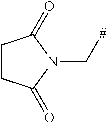 | CH₃ | CH₂F |
| B-1449 | H | H | CF₃ | 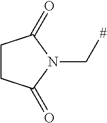 | CH₂F | Br |
| B-1450 | H | H | CF₃ | 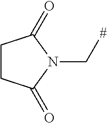 | Br | CH₂F |
| B-1451 | H | H | CF₃ | 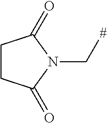 | OCH₃ | CHF₂ |
| B-1452 | H | H | CF₃ | 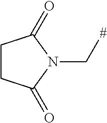 | CHF₂ | OCH₃ |

| | | | | | |
|---|---|---|---|---|---|
| B-1453 | H | H | 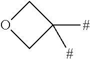 | CH₃ | CH₃ |
| B-1454 | H | H | 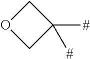 | CH₃ | CHF₂ |
| B-1455 | H | H | 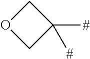 | CHF₂ | CH₃ |
| B-1456 | H | H | 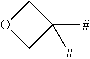 | CH₃ | C≡CH |
| B-1457 | H | H | 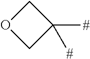 | C≡CH | CH₃ |
| B-1458 | H | H | 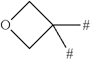 | CH₂F | CH₃ |
| B-1459 | H | H | 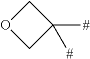 | CH₃ | CH₂F |
| B-1460 | H | H | 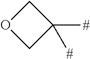 | CH₂F | Br |
| B-1461 | H | H | 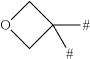 | Br | CH₂F |
| B-1462 | H | H | 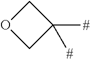 | OCH₃ | CHF₂ |
| B-1463 | H | H | 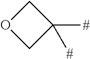 | CHF₂ | OCH₃ |
| B-1464 | H | H | 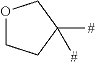 | CH₃ | CH₃ |
| B-1465 | H | H | 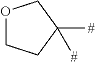 | CH₃ | CHF₂ |
| B-1466 | H | H | 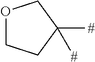 | CHF₂ | CH₃ |
| B-1467 | H | H | 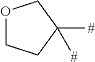 | CH₃ | C≡CH |
| B-1468 | H | H | 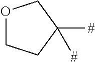 | C≡CH | CH₃ |
| B-1469 | H | H | 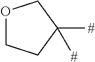 | CH₂F | CH₃ |
| B-1470 | H | H | 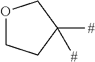 | CH₃ | CH₂F |
| B-1471 | H | H | 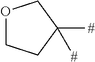 | CH₂F | Br |
| B-1472 | H | H | 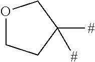 | Br | CH₂F |
| B-1473 | H | H | 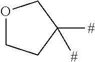 | OCH₃ | CHF₂ |
| B-1474 | H | H | 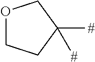 | CHF₂ | OCH₃ |
| B-1475 | H | H | 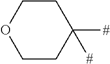 | CH₃ | CH₃ |
| B-1476 | H | H | 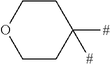 | CH₃ | CHF₂ |
| B-1477 | H | H | 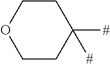 | CHF₂ | CH₃ |
| B-1478 | H | H | 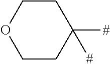 | CH₃ | C≡CH |
| B-1479 | H | H | 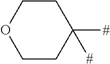 | C≡CH | CH₃ |
| B-1480 | H | H | 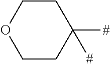 | CH₂F | CH₃ |
| B-1481 | H | H | 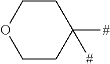 | CH₃ | CH₂F |
| B-1482 | H | H | 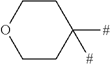 | CH₂F | Br |
| B-1483 | H | H | 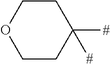 | Br | CH₂F |
| B-1484 | H | H | 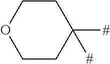 | OCH₃ | CHF₂ |
| B-1485 | H | H | 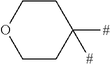 | CHF₂ | OCH₃ |
| B-1486 | H | H | 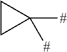 | CH₃ | CH₃ |
| B-1487 | H | H | 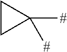 | CH₃ | CHF₂ |
| B-1488 | H | H | 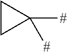 | CHF₂ | CH₃ |
| B-1489 | H | H | 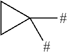 | CH₃ | C≡CH |
| B-1490 | H | H | 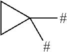 | C≡CH | CH₃ |
| B-1491 | H | H | 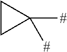 | CH₂F | CH₃ |
| B-1492 | H | H | 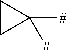 | CH₃ | CH₂F |
| B-1493 | H | H | 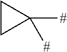 | CH₂F | Br |
| B-1494 | H | H | 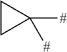 | Br | CH₂F |
| B-1495 | H | H | 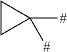 | OCH₃ | CHF₂ |
| B-1496 | H | H | 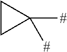 | CHF₂ | OCH₃ |
| B-1497 | H | H | 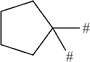 | CH₃ | CH₃ |
| B-1498 | H | H | 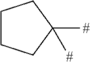 | CH₃ | CHF₂ |
| B-1499 | H | H | 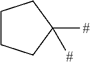 | CHF₂ | CH₃ |
| B-1500 | H | H | 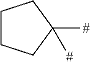 | CH₃ | C≡CH |
| B-1501 | H | H | 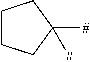 | C≡CH | CH₃ |
| B-1502 | H | H | 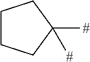 | CH₂F | CH₃ |
| B-1503 | H | H | 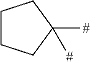 | CH₃ | CH₂F |
| B-1504 | H | H | 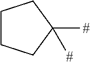 | CH₂F | Br |
| B-1505 | H | H | 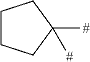 | Br | CH₂F |
| B-1506 | H | H | 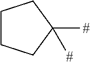 | OCH₃ | CHF₂ |
| B-1507 | H | H | 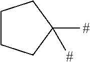 | CHF₂ | OCH₃ |
| B-1508 | H | H | 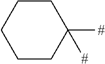 | CH₃ | CH₃ |
| B-1509 | H | H | 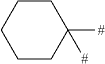 | CH₃ | CHF₂ |
| B-1510 | H | H | 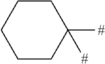 | CHF₂ | CH₃ |
| B-1511 | H | H | 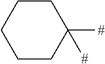 | CH₃ | C≡CH |
| B-1512 | H | H | 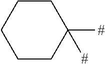 | C≡CH | CH₃ |
| B-1513 | H | H | 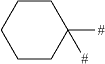 | CH₂F | CH₃ |
| B-1514 | H | H | 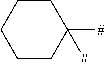 | CH₃ | CH₂F |
| B-1515 | H | H | 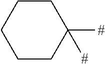 | CH₂F | Br |
| B-1516 | H | H | 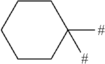 | Br | CH₂F |
| B-1517 | H | H | 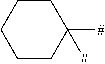 | OCH₃ | CHF₂ |
| B-1518 | H | H | 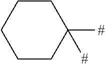 | CHF₂ | OCH₃ |

Particular embodiments of the compounds I are the following compounds I*. In this formula, the substituents R⁹, R¹⁰, R⁷⁸ and o are independently as defined or preferably defined herein:

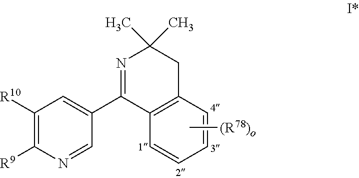

Each of the groups mentioned for a substituent in the tables is furthermore per se, independently of the combination in which it is mentioned, a particularly preferred aspect of the substituent in question.

Particularly preferred embodiments of combinations of R⁹ and R¹⁰ in according to the invention are as compiled in Table B*-1 to B*-8, wherein lines of B*-1 to B*-8 are also in any combination with one another a preferred embodiment of the present invention.

Table B*1 Compounds of formula I* in which o is 1, R⁷⁸ is 4″-CH₃ and the meaning for the combination of R⁹ and R¹⁰ for each individual compound corresponds in each case to one line of Table B*.

Table B*2 Compounds of formula I* in which o is 1, R⁷⁸ is 4″-Cl and the meaning for the combination R⁹ and R¹⁰ for each individual compound corresponds in each case to one line of Table B*.

Table B*3 Compounds of formula I* in which o is 1, R⁷⁸ is 4″-Br and the meaning for the combination R⁹ and R¹⁰ for each individual compound corresponds in each case to one line of Table B*.

Table B*4 Compounds of formula I* in which o is 1, R⁷⁸ is 4″-OCH₃ and the meaning for the combination R⁹ and R¹⁰ for each individual compound corresponds in each case to one line of Table B*.

Table B*5 Compounds of formula I* in which o is 1, R⁷⁸ is 4″-OCF₃ and the meaning for the combination R⁹ and R¹⁰ for each individual compound corresponds in each case to one line of Table B*.

Table B*6 Compounds of formula I* in which o is 1, R⁷⁸ is 4″-OCHF₂ and the meaning for the combination R⁹ and R¹⁰ for each individual compound corresponds in each case to one line of Table B*.

Table B*7 Compounds of formula I* in which o is 1, R⁷⁸ is 4″-CHF₂ and the meaning for the combination R⁹ and R¹⁰ for each individual compound corresponds in each case to one line of Table B*.

Table B*8 Compounds of formula I* in which o is 1, R⁷⁸ is 4″-CF₃ and the meaning for the combination R⁹ and R¹⁰ for each individual compound corresponds in each case to one line of Table B*.

TABLE B*

| line | R⁹ | R¹⁰ |
|---|---|---|
| B*-1 | H | H |
| B*-2 | H | CH₃ |
| B*-3 | H | C₂H₅ |
| B*-4 | H | F |
| B*-5 | H | Cl |
| B*-6 | H | Br |
| B*-7 | H | CN |
| B*-8 | H | OCH₃ |
| B*-9 | H | OCHF₂ |
| B*-10 | H | CHF₂ |
| B*-11 | H | CF₃ |
| B*-12 | H | S—CH₃ |
| B*-13 | H | SO—CH₃ |
| B*-14 | H | SO₂—CH₃ |
| B*-15 | H | CO—NH₂ |
| B*-16 | H | CO—NH(CH₃) |
| B*-17 | H | CO—N(CH₃)₂ |
| B*-18 | CH₃ | H |
| B*-19 | CH₃ | CH₃ |
| B*-20 | CH₃ | C₂H₅ |
| B*-21 | CH₃ | F |
| B*-22 | CH₃ | Cl |
| B*-23 | CH₃ | Br |
| B*-24 | CH₃ | CN |
| B*-25 | CH₃ | OCH₃ |
| B*-26 | CH₃ | OCHF₂ |
| B*-27 | CH₃ | CHF₂ |
| B*-28 | CH₃ | CF₃ |
| B*-29 | CH₃ | S—CH₃ |
| B*-30 | CH₃ | SO—CH₃ |
| B*-31 | CH₃ | SO₂—CH₃ |
| B*-32 | CH₃ | CO—NH₂ |
| B*-33 | CH₃ | CO—NH(CH₃) |
| B*-34 | CH₃ | CO—N(CH₃)₂ |
| B*-35 | C₂H₅ | H |
| B*-36 | C₂H₅ | CH₃ |
| B*-37 | C₂H₅ | C₂H₅ |
| B*-38 | C₂H₅ | F |
| B*-39 | C₂H₅ | Cl |
| B*-40 | C₂H₅ | Br |
| B*-41 | C₂H₅ | CN |
| B*-42 | C₂H₅ | OCH₃ |
| B*-43 | C₂H₅ | OCHF₂ |
| B*-44 | C₂H₅ | CHF₂ |
| B*-45 | C₂H₅ | CF₃ |
| B*-46 | C₂H₅ | S—CH₃ |
| B*-47 | C₂H₅ | SO—CH₃ |
| B*-48 | C₂H₅ | SO₂—CH₃ |
| B*-49 | C₂H₅ | CO—NH₂ |
| B*-50 | C₂H₅ | CO—NH(CH₃) |
| B*-51 | C₂H₅ | CO—N(CH₃)₂ |
| B*-52 | F | H |
| B*-53 | F | CH₃ |
| B*-54 | F | C₂H₅ |
| B*-55 | F | F |
| B*-56 | F | Cl |
| B*-57 | F | Br |
| B*-58 | F | CN |
| B*-59 | F | OCH₃ |
| B*-60 | F | OCHF₂ |
| B*-61 | F | CHF₂ |
| B*-62 | F | CF₃ |
| B*-63 | F | S—CH₃ |
| B*-64 | F | SO—CH₃ |
| B*-65 | F | SO₂—CH₃ |
| B*-66 | F | CO—NH₂ |
| B*-67 | F | CO—NH(CH₃) |
| B*-68 | F | CO—N(CH₃)₂ |
| B*-69 | Cl | H |
| B*-70 | Cl | CH₃ |
| B*-71 | Cl | C₂H₅ |
| B*-72 | Cl | F |
| B*-73 | Cl | Cl |
| B*-74 | Cl | Br |
| B*-75 | Cl | CN |
| B*-76 | Cl | OCH₃ |
| B*-77 | Cl | OCHF₂ |
| B*-78 | Cl | CHF₂ |
| B*-79 | Cl | CF₃ |
| B*-80 | Cl | S—CH₃ |
| B*-81 | Cl | SO—CH₃ |
| B*-82 | Cl | SO₂—CH₃ |
| B*-83 | Cl | CO—NH₂ |
| B*-84 | Cl | CO—NH(CH₃) |
| B*-85 | Cl | CO—N(CH₃)₂ |
| B*-86 | Br | H |
| B*-87 | Br | CH₃ |
| B*-88 | Br | C₂H₅ |
| B*-89 | Br | F |
| B*-90 | Br | Cl |
| B*-91 | Br | Br |
| B*-92 | Br | CN |
| B*-93 | Br | OCH₃ |
| B*-94 | Br | OCHF₂ |
| B*-95 | Br | CHF₂ |
| B*-96 | Br | CF₃ |
| B*-97 | Br | S—CH₃ |
| B*-98 | Br | SO—CH₃ |
| B*-99 | Br | SO₂—CH₃ |
| B*-100 | Br | CO—NH₂ |
| B*-101 | Br | CO—NH(CH₃) |
| B*-102 | Br | CO—N(CH₃)₂ |
| B*-103 | CN | H |
| B*-104 | CN | CH₃ |
| B*-105 | CN | C₂H₅ |
| B*-106 | CN | F |
| B*-107 | CN | Cl |
| B*-108 | CN | Br |
| B*-109 | CN | CN |
| B*-110 | CN | OCH₃ |
| B*-111 | CN | OCHF₂ |
| B*-112 | CN | CHF₂ |
| B*-113 | CN | CF₃ |
| B*-114 | CN | S—CH₃ |
| B*-115 | CN | SO—CH₃ |
| B*-116 | CN | SO₂—CH₃ |
| B*-117 | CN | CO—NH₂ |
| B*-118 | CN | CO—NH(CH₃) |
| B*-119 | CN | CO—N(CH₃)₂ |
| B*-120 | OCH₃ | H |
| B*-121 | OCH₃ | CH₃ |
| B*-122 | OCH₃ | C₂H₅ |
| B*-123 | OCH₃ | F |
| B*-124 | OCH₃ | Cl |
| B*-125 | OCH₃ | Br |
| B*-126 | OCH₃ | CN |
| B*-127 | OCH₃ | OCH₃ |
| B*-128 | OCH₃ | OCHF₂ |
| B*-129 | OCH₃ | CHF₂ |
| B*-130 | OCH₃ | CF₃ |
| B*-131 | OCH₃ | S—CH₃ |
| B*-132 | OCH₃ | SO—CH₃ |
| B*-133 | OCH₃ | SO₂—CH₃ |
| B*-134 | OCH₃ | CO—NH₂ |
| B*-135 | OCH₃ | CO—NH(CH₃) |
| B*-136 | OCH₃ | CO—N(CH₃)₂ |
| B*-137 | OCHF₂ | H |
| B*-138 | OCHF₂ | CH₃ |
| B*-139 | OCHF₂ | C₂H₅ |
| B*-140 | OCHF₂ | F |
| B*-141 | OCHF₂ | Cl |
| B*-142 | OCHF₂ | Br |
| B*-143 | OCHF₂ | CN |
| B*-144 | OCHF₂ | OCH₃ |
| B*-145 | OCHF₂ | OCHF₂ |
| B*-146 | OCHF₂ | CHF₂ |
| B*-147 | OCHF₂ | CF₃ |
| B*-148 | OCHF₂ | S—CH₃ |
| B*-149 | OCHF₂ | SO—CH₃ |
| B*-150 | OCHF₂ | SO₂—CH₃ |
| B*-151 | OCHF₂ | CO—NH₂ |
| B*-152 | OCHF₂ | CO—NH(CH₃) |
| B*-153 | OCHF₂ | CO—N(CH₃)₂ |
| B*-154 | CHF₂ | H |
| B*-155 | CHF₂ | CH₃ |
| B*-156 | CHF₂ | C₂H₅ |
| B*-157 | CHF₂ | F |
| B*-158 | CHF₂ | Cl |
| B*-159 | CHF₂ | Br |
| B*-160 | CHF₂ | CN |
| B*-161 | CHF₂ | OCH₃ |
| B*-162 | CHF₂ | OCHF₂ |
| B*-163 | CHF₂ | CHF₂ |
| B*-164 | CHF₂ | CF₃ |
| B*-165 | CHF₂ | S—CH₃ |
| B*-166 | CHF₂ | SO—CH₃ |
| B*-167 | CHF₂ | SO₂—CH₃ |
| B*-168 | CHF₂ | CO—NH₂ |
| B*-169 | CHF₂ | CO—NH(CH₃) |
| B*-170 | CHF₂ | CO—N(CH₃)₂ |
| B*-171 | CF₃ | H |
| B*-172 | CF₃ | CH₃ |
| B*-173 | CF₃ | C₂H₅ |
| B*-174 | CF₃ | F |
| B*-175 | CF₃ | Cl |
| B*-176 | CF₃ | Br |
| B*-177 | CF₃ | CN |
| B*-178 | CF₃ | OCH₃ |
| B*-179 | CF₃ | OCHF₂ |
| B*-180 | CF₃ | CHF₂ |
| B*-181 | CF₃ | CF₃ |
| B*-182 | CF₃ | S—CH₃ |
| B*-183 | CF₃ | SO—CH₃ |
| B*-184 | CF₃ | SO₂—CH₃ |
| B*-185 | CF₃ | CO—NH₂ |
| B*-186 | CF₃ | CO—NH(CH₃) |
| B*-187 | CF₃ | CO—N(CH₃)₂ |
| B*-188 | S—CH₃ | H |
| B*-189 | S—CH₃ | CH₃ |
| B*-190 | S—CH₃ | C₂H₅ |
| B*-191 | S—CH₃ | F |
| B*-192 | S—CH₃ | Cl |
| B*-193 | S—CH₃ | Br |
| B*-194 | S—CH₃ | CN |
| B*-195 | S—CH₃ | OCH₃ |
| B*-196 | S—CH₃ | OCHF₂ |
| B*-197 | S—CH₃ | CHF₂ |
| B*-198 | S—CH₃ | CF₃ |
| B*-199 | S—CH₃ | S—CH₃ |
| B*-200 | S—CH₃ | SO—CH₃ |
| B*-201 | S—CH₃ | SO₂—CH₃ |
| B*-202 | S—CH₃ | CO—NH₂ |
| B*-203 | S—CH₃ | CO—NH(CH₃) |
| B*-204 | S—CH₃ | CO—N(CH₃)₂ |
| B*-205 | SO—CH₃ | H |
| B*-206 | SO—CH₃ | CH₃ |
| B*-207 | SO—CH₃ | C₂H₅ |
| B*-208 | SO—CH₃ | F |
| B*-209 | SO—CH₃ | Cl |
| B*-210 | SO—CH₃ | Br |
| B*-211 | SO—CH₃ | CN |
| B*-212 | SO—CH₃ | OCH₃ |
| B*-213 | SO—CH₃ | OCHF₂ |
| B*-214 | SO—CH₃ | CHF₂ |
| B*-215 | SO—CH₃ | CF₃ |
| B*-216 | SO—CH₃ | S—CH₃ |
| B*-217 | SO—CH₃ | SO—CH₃ |
| B*-218 | SO—CH₃ | SO₂—CH₃ |
| B*-219 | SO—CH₃ | CO—NH₂ |
| B*-220 | SO—CH₃ | CO—NH(CH₃) |
| B*-221 | SO—CH₃ | CO—N(CH₃)₂ |
| B*-222 | SO₂—CH₃ | H |
| B*-223 | SO₂—CH₃ | CH₃ |
| B*-224 | SO₂—CH₃ | C₂H₅ |
| B*-225 | SO₂—CH₃ | F |
| B*-226 | SO₂—CH₃ | Cl |
| B*-227 | SO₂—CH₃ | Br |
| B*-228 | SO₂—CH₃ | CN |
| B*-229 | SO₂—CH₃ | OCH₃ |
| B*-230 | SO₂—CH₃ | OCHF₂ |
| B*-231 | SO₂—CH₃ | CHF₂ |
| B*-232 | SO₂—CH₃ | CF₃ |
| B*-233 | SO₂—CH₃ | S—CH₃ |
| B*-234 | SO₂—CH₃ | SO—CH₃ |
| B*-235 | SO₂—CH₃ | SO₂—CH₃ |
| B*-236 | SO₂—CH₃ | CO—NH₂ |
| B*-237 | SO₂—CH₃ | CO—NH(CH₃) |
| B*-238 | SO₂—CH₃ | CO—N(CH₃)₂ |
| B*-239 | CO—NH₂ | H |
| B*-240 | CO—NH₂ | CH₃ |
| B*-241 | CO—NH₂ | C₂H₅ |
| B*-242 | CO—NH₂ | F |
| B*-243 | CO—NH₂ | Cl |
| B*-244 | CO—NH₂ | Br |
| B*-245 | CO—NH₂ | CN |
| B*-246 | CO—NH₂ | OCH₃ |
| B*-247 | CO—NH₂ | OCHF₂ |
| B*-248 | CO—NH₂ | CHF₂ |
| B*-249 | CO—NH₂ | CF₃ |
| B*-250 | CO—NH₂ | S—CH₃ |
| B*-251 | CO—NH₂ | SO—CH₃ |
| B*-252 | CO—NH₂ | SO₂—CH₃ |
| B*-253 | CO—NH₂ | CO—NH₂ |
| B*-254 | CO—NH₂ | CO—NH(CH₃) |
| B*-255 | CO—NH₂ | CO—N(CH₃)₂ |
| B*-256 | CO—NH(CH₃) | H |
| B*-257 | CO—NH(CH₃) | CH₃ |
| B*-258 | CO—NH(CH₃) | C₂H₅ |
| B*-259 | CO—NH(CH₃) | F |
| B*-260 | CO—NH(CH₃) | Cl |
| B*-261 | CO—NH(CH₃) | Br |
| B*-262 | CO—NH(CH₃) | CN |
| B*-263 | CO—NH(CH₃) | OCH₃ |
| B*-264 | CO—NH(CH₃) | OCHF₂ |
| B*-265 | CO—NH(CH₃) | CHF₂ |
| B*-266 | CO—NH(CH₃) | CF₃ |
| B*-267 | CO—NH(CH₃) | S—CH₃ |
| B*-268 | CO—NH(CH₃) | SO—CH₃ |
| B*-269 | CO—NH(CH₃) | SO₂—CH₃ |
| B*-270 | CO—NH(CH₃) | CO—NH₂ |
| B*-271 | CO—NH(CH₃) | CO—NH(CH₃) |
| B*-272 | CO—NH(CH₃) | CO—N(CH₃)₂ |
| B*-273 | CO—N(CH₃)₂ | H |
| B*-274 | CO—N(CH₃)₂ | CH₃ |
| B*-275 | CO—N(CH₃)₂ | C₂H₅ |
| B*-276 | CO—N(CH₃)₂ | F |
| B*-277 | CO—N(CH₃)₂ | Cl |
| B*-278 | CO—N(CH₃)₂ | Br |
| B*-279 | CO—N(CH₃)₂ | CN |
| B*-280 | CO—N(CH₃)₂ | OCH₃ |
| B*-281 | CO—N(CH₃)₂ | OCHF₂ |
| B*-282 | CO—N(CH₃)₂ | CHF₂ |
| B*-283 | CO—N(CH₃)₂ | CF₃ |
| B*-284 | CO—N(CH₃)₂ | S—CH₃ |
| B*-285 | CO—N(CH₃)₂ | SO—CH₃ |
| B*-286 | CO—N(CH₃)₂ | SO₂—CH₃ |
| B*-287 | CO—N(CH₃)₂ | CO—NH₂ |
| B*-288 | CO—N(CH₃)₂ | CO—NH(CH₃) |
| B*-289 | CO—N(CH₃)₂ | CO—N(CH₃)₂ |

R⁹, R¹⁰ are independently selected from H, halogen, CN, NO₂, N(R⁹¹)(R⁹²), S(R⁹³), S(P)_{z94}(R⁹⁴), O(R⁹⁵), C₁-C₆-alkyl, C₂-C₆-alkenyl, C₂-C₆-alkynyl, cycloalkyl, CO—(R⁹⁶) and CS—(R⁹⁶);

R⁹¹, R⁹² are independently selected from H, alkyl, alkenyl, alkynyl, cycloalkyl, carbonyl-R(⁹¹¹), S(O)_{z91}R⁹¹²;

R⁹¹¹ is H or R⁹¹²;

R⁹¹² is independently selected from alkyl, alkenyl, alkynyl, cycloalkyl, O—R⁹¹¹¹, N(R⁹¹¹²)(R⁹¹¹³);

R⁹¹¹¹ is alkyl, alkenyl, alkynyl or cycloalkyl;

R⁹¹¹² R⁹¹¹³ are independently selected from H, alkyl, alkenyl, alkynyl and cycloalkyl; ₉₁ is 1 or 2;

R⁹³ is independently selected from H, alkyl, alkenyl, alkynyl, cycloalkyl;

R⁹⁴ is independently selected from alkyl, alkenyl, alkynyl, cycloalkyl, O—R⁹⁴¹, N(R⁹⁴²)(R⁹⁴³);

R⁹⁴², R⁹⁴³ are independently selected from H or R⁹⁴¹;

R⁹⁴¹ is independently selected from alkyl, alkenyl, alkynyl, cycloalkyl;

_{z94} is1 or 2;

R⁹⁵ is independently selected from H, alkyl, alkenyl, alkynyl, cycloalkyl, carbonyl-R(⁹⁵¹), S(O)_{z95}(R⁹⁵²);

R⁹⁵¹ is H or R⁹⁵²;

R⁹⁵² is independently selected from alkyl, alkenyl, alkynyl, cycloalkyl, O—R⁹⁵²¹, N(R⁹⁵²²)(R⁹⁵²³);

R⁹⁵²¹ is independently selected from alkyl, alkenyl, alkynyl, cycloalkyl;

R⁸⁹⁵²², R⁸⁹⁵²³ is independently selected from H and R⁹⁵²¹;

_{z95} is 1 or 2;

R⁹⁶ is independently selected from H, alkyl, alkenyl, alkynyl, cycloalkyl, O—R⁹⁶¹, N(R⁹⁶²)(R⁹⁶³);

R⁹⁶¹ is independently selected from H, alkyl, alkenyl, alkynyl, cycloalkyl;

R⁹⁶², R⁹⁶³ are independently selected from H, alkyl, alkenyl, alkynyl, cycloalkyl;

wherein the acyclic moieties of R⁹ and R¹⁰ are independently unsubstituted or substituted; and with identical or different groups R^{9a} or R^{10a}, respectively, which independently of one another are selected from:

R^{9a}, R^{10a} halogen, OH, CN, C₁-C₆-alkoxy, alkenyloxy, alkynyloxy, C₃-C₆-cycloalkyl, C₃-C₆-halocycloalky, C₁-C₄-haloalkoxy, C₁-C₆-alkylthio; or

R⁹ and R¹⁰ together with the carbon atoms to which they are bound form a five- , six-, or seven-membered carbo- and heterocyclic or heteroaromatic ring; wherein the ring contains one, two, three or four heteroatoms selected from N, O and S, wherein the heteroatom N may carry one substituent R^{N} selected from C₁-C₄-alkyl, C₁-C₄-haloalkyl and SO₂Ph, wherein Ph is unsubstituted phenyl or phenyl that is substituted by one, two or three substituents selected from C₁-C₄-alkyl, and wherein the heteroatom S may be in the form of its oxide SO or SO₂; and wherein in each case one or two CH₂ groups of the carbo- or heterocycle may be replaced by a group independently selected from C(═O) and C(═S); and wherein the carbo- and heterocyclic ring is substituted by (R¹¹)ₘ, wherein m is 0, 1, 2, 3 or 4;

R¹¹ is in each case independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from N, O and S; and wherein

R^{x} is C₁-C₄-alkyl, C₁-C₄-haloalkyl, unsubstituted aryl or aryl that is substituted by one, two, three, four or five substituents R^{x1} independently selected from C₁-C₄-alkyl;

wherein in each case one or two CH₂ groups of cabocycle or heterocycle of R¹¹ may be replaced by a group independently selected from C(═O) and C(═S); and wherein the acyclic moieties of R¹¹ are unsubstituted or substituted with identical or different groups R^{11a} which independently of one another are selected from:

R^{11a} halogen, OH, CN, C₁-C₆-alkoxy, C₃-C₆-cycloalkyl, C₃-C₆-halocycloalkyl, C₁-C₄-haloalkoxy, C₁-C₆-alkylthio and phenoxy, wherein the phenyl group is unsubstituted or unsubstituted or substituted with R¹¹a selected from the group consisting of halogen, OH, C₁-C₄-alkyl, C₁-C₄-haloalkyl, C₁-C₄-alkoxy and C₁-C₄-haloalkoxy;

wherein the cycloalkyl, heteroaryl and aryl moieties of R¹¹ are unsubstituted or substituted with identical or different groups R^{11b} which independently of one another are selected from:

R^{11b} halogen, OH, CN, C₁-C₄-alkyl, C₁-C₄-alkoxy, C₁-C₄-haloalkyl, C₃-C₆-cycloalkyl, C₃-C₆-halocycloalky, C₁-C₄-haloalkoxy and C₁-C₆-alkylthio.

In still another embodiment of formula I, R⁹ is selected from H, halogen, OH, CN, SH, C₁-C₆-alkylthio, NH₂, NH(C₁-C₄-alkyl), N(C₁-C₄-alkyl)₂, NH—SO₂—R⁹¹², C₁-C₆-alkyl, C₂-C₆-alkenyl, C₂-C₆-alkynyl, and C₁-C₆-alkoxy, in particular halogen, OH, CN, C₁-C₄-alkylthio, C₁-C₆-alkyl, C₂-C₆-alkenyl, C₂-C₆-alkynyl, C₁-C₆-alkoxy and, wherein R⁹¹² is defined above; and wherein the acyclic moieties of R⁹ are unsubstituted or substituted with identical or different groups R⁹a as defined above.

In still another embodiment of formula I, R⁹ is selected from H, OH, CN, SH, C₁-C₆-alkylthio, NH₂, NH(C₁-C₄-alkyl), N(C₁-C₄-alkyl)₂, NH—SO₂—R⁹¹², C₁-C₆-alkyl, C₂-C₆-alkenyl, C₂-C₆-alkynyl, and C₁-C₆-alkoxy, in particular OH, CN, C₁-C₄-alkylthio, C₁-C₆-alkyl, C₂-C₆-alkenyl, C₂-C₆-alkynyl, and C₁-C₆-alkoxy , wherein R⁹¹² is defined below; and wherein the acyclic moieties of R⁹ are unsubstituted or substituted with identical or different groups R⁹a as defined above.

In still another embodiment of formula I, R⁹ is halogen or C₁-C₆-alkyl, such as Cl, F, Br, methyl, ethyl, n-propyl, i-propyl, n-butyl, i-butyl, tert-butyl, n-pentyl or i-pentyl.

In still another embodiment of formula I, R⁹ is C₁-C₆-alkyl, such as methyl, ethyl, n-propyl, i-propyl, n-butyl, i-butyl, tert-butyl, n-pentyl or i-pentyl, more specificall methyl and ethyl.

In still another embodiment of formula I, R⁹ is C₁-C₆-alkyl, such as methyl.

In still another embodiment of formula I, R⁹ is CN, S-alkyl, S-alkynyl or S-alkenyl.

In still another embodiment of formula I, R⁹ is NO₂, NH₂, SO₂, OH, OCO-alkyl, OCO-alkenyl or OCO-alkynyl.

In still another embodiment of formula I, R⁹ is halogen, CN, NO₂, NH₂, CO-alkyl, CS-alkyl, CO—NH₂, CO—NH(CH₃); CO—N(CH₃)₂, CS—NH₂, CS—NH(CH₃), CS—N(CH₃)₂, CO—N(CH₃)₂, SO₂, OH, OCO-alkyl, OCO-alkenyl or OCO-alkynyl, S-(alkyl)₃, SO-(alkyl)₂, SO₂-alkyl, SO-(alkenyl)₂, SO₂-alkenyl, SO-(alkynyl)₂ or SO₂-alkynyl.

In still another embodiment of formula I, R⁹ is S-(alkyl)₃ such as S—CH₃.

In still another embodiment of formula I, R⁹ is SO-(alkyl)₂ such as SO—CH₃.

In still another embodiment of formula I, R⁹ is SO₂alkyl such as SO₂—CH₃.

In still another embodiment of formula I, R⁹ is alkoxy or halogen substituted alkoxy such as OCH₃, OCF₃, OCHF₂, OCH₂F, OCCl₃, OCHCl₂ or OCH₂Cl.

In still another embodiment of formula I, R⁹ is alkyl or a halogen substituted alkyl such as CH₃, C₂H₅, CHF₂, CF₃, CH₂CF₃ or CF₂CF₃.

In still another embodiment of formula I, R⁹ is alkenyl or a halogen substituted alkenyl, such as CH═CH₂, C(CH₃)═CH₂, CH₂CH═CH₂, CH═CHF, CH═CHCl, CH═CF₂, CH═CCl₂, CH₂CH═CHF, CH₂CH═CHCl, CH₂CH═CF₂, CH₂CH═CCl₂, CF₂CH═CF₂, CCl₂CH═CCl₂, CF₂CF═CF₂, CCl₂CCl=CCl₂.

In still another embodiment of formula I, R⁹ is alkynyl or a halogen substituted alkynyl, such as C≡CH, CH₂C≡CH.

In still another embodiment of formula I, R⁹ is CN.

In still another embodiment of formula I, R⁹ is H.

In still another embodiment of formula I, R⁹ is CN, cycloalkyl such as cyclopropyl, cyclobutyl, cyclopentyl, cyclohexyl, cycloheptyl wherin the cycloalkyl may contain one or two heteroatoms such as O, S and N.

In still another embodiment of formula I, R⁹ is cycloalkyl such as cyclopropyl.

In still another embodiment of formula I, R⁹ is C₃-C₆-halocycloalkyl. In a special embodiment R^{9b} is fully or partially halogenated cyclopropyl, such as 1-F-cyclopropyl, 1-Cl-cyclopropyl.

In still another embodiment of formula I, R⁹ is halogen such as F, Br, Cl or I.

In still another embodiment of formula I, R⁹ is F.

In still another embodiment of formula I, R⁹ is Cl.

In still another embodiment of formula I, R⁹ is Br.

In still another embodiment of formula I, R⁹ is OH.

In still another embodiment of formula I, R⁹ is NO₂.

In still another embodiment of formula I, R⁹ is is S(R⁹³) such as SH.

In still another embodiment of formula I, R⁹ is S(R⁹³) such as S—C₁-C₆-alkyl such as SCH₃ or SCH₂CH₃.

In still another embodiment of formula I, R⁹ is S(R⁹³) scuh as S—C₂-C₆-alkenyl such as SCH═CH₂, SCH₂CH═CH₂.

According to still another embodiment of formula I, R⁹ is S(R⁹³) such as S C₂-C₆-alkynyl such as SC≡CH, SCH₂C≡CH.

In still another embodiment of formula I, R⁹ is N(R⁹¹)(R⁹²) such as NH₂.

In still another embodiment of formula I, R⁹ is NH-C₁-C₆-alkyl in particular NH—CH₂.

In still another embodiment of formula I, R⁹ is ,N(R⁹¹)(R⁹²) such as N(C₁-C₄-alkyl)₂, in particular NH(CH₃)₂, NH(C₂H₅)₂.

In still another embodiment of formula I, R⁹ is N(R⁹¹)(R⁹²) such as, NH(C₂-C₄-alkenyl), in particular NH(CH═CH₂), NH(CH₂CH═CH₂).

In still another embodiment of formula I, R⁹ is N(R⁹¹)(R⁹²) such as N(C₂-C₄-alkenyl)₂, in particular N(CH═CH₂)₂, N(CH₂CH═CH₂)₂.

In still another embodiment of formula I, R⁹ is N(R⁹¹)(R⁹²) such as NH(C₂-C₄-alkynyl), in particular NH(C≡CH), NH(CH₂C≡CH).

In still another embodiment of formula I, R⁹ is , N(R⁹¹)(R⁹²) such as N(C₂-C₄-alkynyl)₂, in particular N(C≡CH)₂, N(CH₂C≡CH)₂.

In still another embodiment of formula I, R⁹ is N(R⁹¹)(R⁹²) such as NH(C₃-C₆-cycloalkyl), in particular NH(C₃H₇), NH(C₄H₉).

In still another embodiment of formula I, R⁹ is N(R⁹¹)(R⁹²) such as, N(C₃-C₆-cycloalkyl)₂, in particular N(C₃H₇)₂, N(C₄H₉)₂.

In still another embodiment of formula I, R⁹ is N(R⁹¹)(R⁹²) such as N(C₁-C₄-alkyl)(C₂-C₄-alkenyl), in particular N(CH₃)(CH═CH₂), N(CH₃)(CH₂CH═CH₂), N(C₂H₅)(CH═CH₂), N(C₂H₅)(CH₂CH═CH₂).

In still another embodiment of formula I, R⁹ is N(R⁹¹)(R⁹²) such as N(C₁-C₄-alkyl)(C₂-C₄-alkynyl), in particular N(CH₃)(C≡CH), N(CH₃)(CH₂C≡CH), N(C₂H₅)(C≡CH), N(C₂H₅)(CH₂C≡CH).

In still another embodiment of formula I, R⁹ is N(R⁹¹)(R⁹²) such as N(C₁-C₄-alkyl)(C3-C₆-cycloalkyl), in particular N(CH₃)(C₃H₇), N(CH₃)(C₄H₉), N(C₂H₅)(C₃H₇), N(CH₃)(C₄H₉).

In still another embodiment of formula I, R⁹ is N(R⁹¹)(R⁹²) such as N(C₂-C₄-alkenyl) (C₂-C₄-alkynyl), in particular N(CH═CH₂)(C≡CH), N(CH₂CH═CH₂)(CH₂C≡CH), N(CH═CH₂)(C≡CH), N(CH₂CH═CH₂)(CH₂C≡CH).

In still another embodiment of formula I, R⁹ is N(R⁹¹)(R⁹²) such as N(C2-C₄-alkenyl)(C3-C₆-cycloalkyl), in particular N(CH═CH₂)(C₃H₇), N(CH₂CH═CH₂)(C₄H₉), N(CH═CH₂)(C₃H₇), N(CH₂CH═CH₂)(C₄H₉).

In still another embodiment of formula I, R⁹ is N(R⁹¹)(R⁹²) such as N(C₂-C₄-alkynyl)(C₃-C₆-cycloalkyl), in particular N(C≡CH)(C₃H₇), N(CH₂C≡CH)(C₄H₉), N(C≡CH)(C₃H₇), N(CH₂C≡CH)(C₄H₉).

In still another embodiment of formula I, R⁹ is N(R⁹¹)(R⁹²) such as NH(C(═O)(C₁-C₄-alkyl), in particular NH(C(═O)(CH₃), NH(C(═O)(C₂H₅).

In still another embodiment of formula I, R⁹ is N(R⁹¹)(R⁹²) such as N(C(═O)(C₁-C₄-alkyl)2, in particular N(C(═O)(CH₃)₂, N(C(═O)(C2H5)₂.

In a further specific embodiment of formula I, R⁹ is N(R⁹¹)(R⁹²) such as NH—SO₂—R^{x} such as NH—SO₂—CH₃, NH—SO₂—CH₂—CH₃, NH—SO₂—CF₃, NH—SO₂-Ts.

In still another embodiment of formula I, R⁹ is S(O)_{z94}(R⁹⁴) such as S(O)_{z94}—C₁-C₆-alkyl such as S(═O) CH₃, S(O)₂CH₃.

In still another embodiment of formula I, R⁹ is S(O)_{z94}(R⁹⁴) scuh as S(O)_{z94}—C₂-C₆-alkenyl such as S(═O)CH═CH₂, S(O)₂CH═CH₂, S(═O)CH₂CH═CH₂, S(O)₂CH₂CH═CH₂.

According to still another embodiment of formula I, R⁹ is S(O)_{z94}(R⁹⁴) such as S(O)_{z94}—C₂-C₆-alkynyl such as S(═O)C≡CH, S(O)₂C≡CH, S(═O)CH₂C≡CH, S(O)₂CH₂C≡CH.

In a further specific embodiment of formula I, R⁹ is CO(R⁹⁶) such as CH(═O).

In a further specific embodiment of formula I, R⁹ is CO(R⁹⁶) such as C(═O)C₁-C₆-alkyl, C(═O)O(C₁-C₆-alkyl) or C(═O)NH(C₁-C₆-alkyl), wherein alkyl is CH₃, C₂H₅, n-propyl, i-propyl, n-butyl, i-butyl, tert-butyl, n-pentyl or i-pentyl.

In a further specific embodiment of formula I, R⁹ is CO(R⁹⁶) such as C(═O)C₂-C₆-alkenyl, C(═O)O(C₂-C₆-alkenyl) or C(═O)NH(C₂-C₆-alkenyl), wherein alkenyl is CH═CH₂, CH₂CH═CH₂.

In a further specific embodiment of formula I, R⁹ is CO(R⁹⁶) such as C(═O)C₂-C₆-alkynyl, C(═O)O(C₂-C₆-alkynyl) or C(═O)NH(C₂-C₆-alkynyl), wherein alkynyl is C≡CH, CH₂C≡CH.

In a further specific embodiment of formula I, R⁹ is CO(R⁹⁶) such as C(═O)C₃-C₆-cycloalkyl, C(═O)O(C₃-C₆-cycloalkyl) or C(═O)NH(C₃-C₆-cycloalkyl), wherein cycloalkyl is cyclopropyl (C₃H₇) or cyclobutyl (C₄H₉).

In a further specific embodiment of formula I, R⁹ is CS(R⁹⁶) such as CH(═S).

According to a further specific embodiment of formula I, R⁹ is CS(R⁹⁶) such as C(═S)C₁-C₆-alkyl, wherein alkyl is CH₃, C₂H₅, n-propyl, i-propyl, n-butyl, i-butyl, tert-butyl, n-pentyl or i-pentyl.

In a further specific embodiment of formula I, R⁹ is CS(R⁹⁶) such as C(═S)C₂-C₆-alkenyl, wherein alkenyl is CH═CH₂, CH₂CH═CH₂.

In a further specific embodiment of formula I, R⁹ is CS(R⁹⁶) such as C(═S)C₂-C₆-alkynyl, wherein alkynyl is C≡CH, CH₂C≡CH.

In a further specific embodiment of formula I, R⁹ is CS(R⁹⁶) such as C(═S)C₃-C₆-cycloalkyl, wherein cycloalkyl is cyclopropyl (C₃H₇) or cyclobutyl (C₄H₉).

In a further specific embodiment of formula I, R⁹ is CS(R⁹⁶) such as C(═S)NHC₁-C₆-alkyl, wherein alkyl is CH₃, C₂H₅, n-propyl, i-propyl, n-butyl, i-butyl, tert-butyl, n-pentyl or i-pentyl.

In still another embodiment of formula I, R⁹ is C₁-C₆-alkoxy, such as OCH₃, OC₂H₅, propoxy, n-butoxy and tert-butoxy, more specifically OCH₃.

In a further specific embodiment of formula I, R⁹ is OR⁹⁵, wherein R⁹⁵ is C₂-C₆-alkenyl, in particular C₂-C₄-alkenyl, more specifically C₁-C₂-alkenyl. R⁹ is such as OCH═CH₂, OCH₂CH═CH₂.

In a further specific embodiment of formula I, R⁹ is OR⁹⁵, wherein R⁹⁵ is C₂-C₆-alkynyl, in particular C₂-C₆-alkynyl, in particular C₂-C₄-alkynyl, more specifically C₁-C₂-alkynyl. R⁹ is such as OC≡CH

In still another embodiment of formula I R⁹ is OR⁹⁵, wherein R⁹⁵ is C₃-C₆-cycloalkyl, in particular cyclopropyl.

In still another embodiment of formula I, R⁹ is OR⁹⁵, wherein R⁹⁵ is C₃-C₆-halocycloalkyl. In a special embodiment R¹ is fully or partially halogenated cyclopropyl.

In still another embodiment of formula I, R⁹ is is OR⁹⁵, wherein R⁹⁵ C₃-C₆-cycloalkenyl, in particular cyclopropenyl.

In still another embodiment of formula I, R⁹ is an amide goup such as CO—N H₂, CO—NH(CH₃); CO—N(CH₃)₂.

Particularly preferred embodiments of R⁹ according to the invention are in Table P13 below, wherein each line of lines P13-1 to P3-40 corresponds to one particular embodiment of the invention, wherein P13-1 to P3-40 are also in any combination with one another a preferred embodiment of the present invention. The connection point to the carbon atom, to which R⁹ is bound is marked with “#” in the drawings.

TABLE P13

| No. | R⁹ |
|---|---|
| P13-1 | H |
| P13-2 | CH₃ |
| P13-3 | CHF₂ |
| P13-4 | CF₃ |
| P13-5 | C₂H₅ |
| P13-6 | CH(CH₃)₂ |
| P13-7 | CH₂CH₂CH₃ |
| P13-8 | CH₂CH₂CH₂CH₃ |
| P13-9 | CH₂CH(CH₃)₂ |
| P13-10 | C(CH₃)₃ |
| P13-11 | CH₂CH₂CH₂CH₂CH₃ |
| P13-12 | CH₂CH₂CH(CH₃)₂ |
| P13-13 | SO(CH₃)₂ |
| P13-14 | OH |
| P13-15 | OCH₃ |
| P13-16 | OCHF₂ |
| P13-17 | OC₂H₅ |
| P13-18 | CN |
| P13-19 | F |
| P13-20 | Cl |
| P13-21 | Br |
| P13-22 | NO₂ |
| P13-23 | NH₂ |
| P13-24 | CO—NH₂ |
| P13-25 | CO—NH(CH₃) |
| P13-26 | CO—N(CH₃)₂ |
| P13-27 | HNCH₃ |
| P13-28 | HNC₂H₅ |
| P13-29 | C≡CH |
| P13-30 | SO₂ |
| P13-31 | SO₂—CH₃ |
| P13-32 | SO—CH₃ |
| P13-33 | S—CH₃ |
| P13-34 | 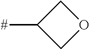 |
| | (C1) |
| P13-35 | 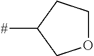 |
| | (C2) |
| P13-36 | 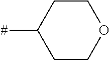 |
| | (C3) |
| P13-37 | 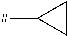 |
| | (C4) |
| P13-38 | 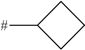 |
| | (C5) |
| P13-39 | 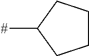 |
| | (C6) |
| P13-40 | 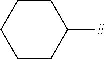 |
| | (C7 |

In still another embodiment of formula I, R¹⁰ is selected from H, halogen, OH, CN, SH, C₁-C₆-alkylthio, NH₂, NH(C₁-C₄-alkyl), N(C₁-C₄-alkyl)₂, NH—SO₂—R⁹¹², C₁-C₆-alkyl, C₂-C₆-alkenyl, C₂-C₆-alkynyl, and C₁-C₆-alkoxy, in particular halogen, OH, CN, C₁-C₄-alkylthio, C₁-C₆-alkyl, C₂-C₆-alkenyl, C₂-C₆-alkynyl, C₁-C₆-alkoxy and, wherein R⁹¹² is defined above; and wherein the acyclic moieties of R¹⁰ are unsubstituted or substituted with identical or different groups R^{10a} as defined above.

In still another embodiment of formula I, R¹⁰ is selected from H, OH, CN, SH, C₁-C₆-alkylthio, NH₂, NH(C₁-C₄-alkyl), N(C₁-C₄-alkyl)₂, NH—SO₂-R⁹¹², C₁-C₆-alkyl, C₂-C₆-alkenyl, C₂-C₆-alkynyl, and C₁-C₆-alkoxy, in particular OH, CN, C₁-C₄-alkylthio, C₁-C₆-alkyl, C₂-C₆-alkenyl, C₂-C₆-alkynyl, and C₁-C₆-alkoxy , wherein R⁹¹² is defined above; and wherein the acyclic moieties of R¹⁰ are unsubstituted or substituted with identical or different groups R^{10a} as defined above.

In still another embodiment of formula I, R¹⁰ is halogen or C₁-C₆-alkyl, such as Cl, F, Br, CH₃, C₂H₅, n-propyl, i-propyl, n-butyl, i-butyl, tert-butyl, n-pentyl or i-pentyl.

In still another embodiment of formula I, R¹⁰ is C₁-C₆-alkyl, such as CH₃, C₂H₅, n-propyl, propyl, n-butyl, i-butyl, tert-butyl, n-pentyl or i-pentyl, more specificall methyl and ethyl.

In still another embodiment of formula I, R⁹ is C₁-C₆-alkyl, such as methyl.

In still another embodiment of formula I, R¹⁰ is CN, S-alkyl, S-alkynyl or S-alkenyl.

In still another embodiment of formula I, R¹⁰ is NO₂, NH₂, SO₂, OH, OCO-alkyl, OCO-alkenyl or OCO-alkynyl.

In still another embodiment of formula I, R¹⁰ is halogen, CN, NO₂, NH₂, CO-alkyl, CS-alkyl, CO—NH₂, CO—NH(CH₃); CO—N(CH₃)₂, CS—NH₂, CS—NH(CH₃); CS—N(CH₃)₂, SO₂, OH, OCO-alkyl, OCO-alkenyl or OCO-alkynyl, S-(alkyl)₃, SO-(alkyl)₂, SO₂-alkyl, SO-(alkenyl)₂, SO₂-alkenyl, SO-(alkynyl)₂ or SO₂-alkynyl.

In still another embodiment of formula I, R¹⁰ is S-(alkyl)₃ such as S—CH₃.

In still another embodiment of formula I, R¹⁰ is SO-(alkyl)₂ such as SO—CH₃.

In still another embodiment of formula I, R¹⁰is SO2 alkyl such as SO₂—CH₃.

In still another embodiment of formula I, R¹⁰ is alkoxy or halogen substituted alkoxy such as OCH₃ or OCHF₂.

In still another embodiment of formula I, R¹⁰ is alkyl or a halogen substituted alkyl such as CH₃, C₂H₅, CHF₂, CF₃, CH₂CF₃ or CF₂CF₃.

In still another embodiment of formula I, R¹⁰ is alkenyl or a halogen substituted alkenyl, such as CH═CH₂, C(CH₃)═CH₂, CH₂CH═CH₂, CH═CHF, CH═CHCl, CH═CF₂, CH═CCl₂, CH₂CH═CHF, CH₂CH═CHCl, CH₂CH═CF₂, CH₂CH═CCl₂, CF₂CH═CF₂, CCl₂CH═CCl₂, CF₂CF═CF₂, CCl₂CCl=CCl₂.

In still another embodiment of formula I, R¹⁰ is alkynyl or a halogen substituted alkynyl, such as C≡CH or CH₂C≡CH.

In still another embodiment of formula I, R¹⁰ is CN.

In still another embodiment of formula I, R¹⁰ is H.

In still another embodiment of formula I, R¹⁰ is CN, cycloalkyl such as cyclopropyl, cyclobutyl, cyclopentyl, cyclohexyl, cycloheptyl wherin the cycloalkyl may contain one or two heteroatoms such as O, S and N.

In still another embodiment of formula I, R¹⁰ is cycloalkyl such as cyclopropyl.

In still another embodiment of formula I, R¹⁰ is C₃-C₆-halocycloalkyl. In a special embodiment R^{9b} is fully or partially halogenated cyclopropyl, such as 1-F-cyclopropyl, 1-Cl-cyclopropyl.

In still another embodiment of formula I, R⁹ is halogen such as F, Br, Cl or I.

In still another embodiment of formula I, R¹⁰ is F.

In still another embodiment of formula I, R¹⁰ is Cl.

In still another embodiment of formula I, R¹⁰ is Br.

In still another embodiment of formula I, R¹⁰ is OH.

In still another embodiment of formula I, R¹⁰ is NO₂.

In still another embodiment of formula I, R¹⁰ is S(R⁹³) such as SH.

In still another embodiment of formula I, R¹⁰ is N(R⁹¹)(R⁹²) such as NH₂.

In still another embodiment of formula I, R¹⁰ is S(R⁹³) such as S—C₁-C₆-alkyl such as SCH₃ or SCH₂CH₃.

In still another embodiment of formula I, R¹⁰ is S(R⁹³) scuh as S—C₂-C₆-alkenyl such as SCH═CH₂, SCH₂CH═CH₂.

According to still another embodiment of formula I, R¹⁰ is S(R⁹³) such as S C₂-C₆-alkynyl such as SC≡CH, SCH₂C≡CH.

In still another embodiment of formula I, R¹⁰ is NH—C₁-C₆-alkyl or NH—CH₂.

In still another embodiment of formula I, R¹⁰ is N(R⁹¹)(R⁹²) such as, N(C₁-C₄-alkyl)₂, in particular NH(CH₃)₂, NH(C₂H₅)₂.

In still another embodiment of formula I, R¹⁰ is N(R⁹¹)(R⁹²) such as NH(C₂-C₄-alkenyl), in particular NH(CH═CH₂), NH(CH₂CH═CH₂).

In still another embodiment of formula I, R¹⁰ is N(R⁹¹)(R⁹²) such as N(C₂-C₄-alkenyl)₂, in particular N(CH═CH₂)₂, N(CH₂CH═CH₂)₂.

In still another embodiment of formula I, R¹⁰ is N(R⁹¹)(R⁹²) such as NH(C₂-C₄-alkynyl), in particular NH(C≡CH), NH(CH₂C≡CH).

In still another embodiment of formula I, R¹⁰ is N(R⁹¹)(R⁹²) such as N(C₂-C₄-alkynyl)₂, in particular N(C≡CH)₂, N(CH₂C≡CH)₂.

In still another embodiment of formula I, R¹⁰ is N(R⁹¹)(R⁹²) such as NH(C₃-C₆-cycloalkyl), in particular NH(C₃H₇), NH(C₄H₉).

In still another embodiment of formula I, R¹⁰ is N(R⁹¹)(R⁹²) such as N(C₃-C₆-cycloalkyl)₂, in particular N(C₃H₇)₂, N(C₄H₉)₂.

In still another embodiment of formula I, R¹⁰ is N(R⁹¹)(R⁹²) such as N(C₁-C₄-alkyl)(C2-C₄-alkenyl), in particular N(CH₃)(CH═CH₂), N(CH₃)(CH₂CH═CH₂), N(C₂H₅)(CH═CH₂), N(C₂H₅)(CH₂CH═CH₂).

In still another embodiment of formula I, R¹⁰ is N(R⁹¹)(R⁹²) such as N(C₁-C₄-alkyl)(C₂-C₄-alkynyl), in particular N(CH₃)(C≡CH), N(CH₃)(CH₂C≡CH), N(C₂H₅)(C≡CH), N(C₂H₅)(CH₂C≡CH).

In still another embodiment of formula I, R¹⁰ is N(R⁹¹)(R⁹²) such as N(C₁-C₄-alkyl)(C3-C₆-cycloalkyl), in particular N(CH₃)(C₃H₇), N(CH₃)(C₄H₉), N(C₂H₅)(C₃H₇), N(CH₃)(C₄H₉).

In still another embodiment of formula I, R¹⁰ is N(R⁹¹)(R⁹²) such as N(C2-C₄-alkenyl) (C2-C₄-alkynyl), in particular N(CH═CH₂)(C≡CH), N(CH₂CH═CH₂)(CH₂C≡CH), N(CH═CH₂)(C≡CH), N(CH₂CH═CH₂)(CH₂C≡CH).

In still another embodiment of formula I, R¹⁰ is N(R⁹¹)(R⁹²) such as N(C₂-C₄-alkenyl)(C3-C₆-cycloalkyl), in particular N(CH═CH₂)(C₃H₇), N(CH₂CH═CH₂)(C₄H₉), N(CH═CH₂)(C₃H₇), N(CH₂CH═CH₂)(C₄H₉).

In still another embodiment of formula I, R⁹ is N(R⁹¹)(R⁹²) such as N(C₂-C₄-alkynyl)(C₃-C₆-cycloalkyl), in particular N(C≡CH)(C₃H₇), N(CH₂C≡CH)(C₄H₉), N(C≡CH)(C₃H₇), N(CH₂C≡CH)(C₄H₉).

In still another embodiment of formula I, R¹⁰ is ,N(R⁹¹)(R⁹²) such as NH(C(═O)(C₁-C₄-alkyl), in particular NH(C(═O)(CH₃), NH(C(═O)(C₂H₅).

In still another embodiment of formula I, R¹⁰ is N(R⁹¹)(R⁹²) such as N(C(═O)(C₁-C₄-alkyl)₂, in particular N(C(═O)(CH₃)₂, N(C(═O)(C₂H₅)₂.

In a further specific embodiment of formula I, R¹⁰ is N(R⁹¹)(R⁹²) such as NH—SO₂—R^{x} such as NH—SO₂—CH₃, NH—SO₂—CH₂—CH₃, NH—SO₂—CF₃, NH—SO₂-Ts.

In still another embodiment of formula I, R⁹ is an amide goup such as CO—N H2, CO—NH(CH₃); CO—N(CH₃)₂.

In still another embodiment of formula I, R¹⁰ is S(O)_{z94}(R⁹⁴) such as S(O)_{z94}-C₁-C₆-alkyl such as S(═O) CH₃, S(O)₂CH₃.

In still another embodiment of formula I, R¹⁰ is S(O)_{z94}(R⁹⁴) such as S(O)_{z94}-C₂-C₆-alkenyl such as S(═O)CH═CH₂, S(O)₂CH═CH₂, S(═O)CH₂CH═CH₂, S(O)₂CH₂CH═CH₂.

According to still another embodiment of formula I, R¹⁰ is S(O)_{z94}(R⁹⁴) such as S(O)_{z94}-C₂-C₆-alkynyl such as S(═O)C≡CH, S(O)₂C≡CH, S(═O)CH₂C≡CH, S(O)₂CH₂C≡CH.

In a further specific embodiment of formula I, R¹⁰ is CO(R⁹⁶) such as CH(═O).

In a further specific embodiment of formula I, R¹⁰ is CO(R⁹⁶) such as C(═O)C₁-C₆-alkyl, C(═O)O(C₁-C₆-alkyl) or C(═O)NH(C₁-C₆-alkyl), wherein alkyl is CH₃, C₂H₅, n-propyl, i-propyl, n-butyl, i-butyl, tert-butyl, n-pentyl or i-pentyl.

In a further specific embodiment of formula I, R¹⁰ is CO(R⁹⁶) such as C(═O)C₂-C₆-alkenyl, C(═O)O(C₂-C₆-alkenyl) or C(═O)NH(C₂-C₆-alkenyl), wherein alkenyl is CH═CH₂, CH₂CH═CH₂.

In a further specific embodiment of formula I, R¹⁰ is CO(R⁹⁶) such as C(═O)C₂-C₆-alkynyl, C(═O)O(C₂-C₆-alkynyl) or C(═O)NH(C₂-C₆-alkynyl), wherein alkynyl is C≡CH, CH₂C≡CH.

In a further specific embodiment of formula I, R¹⁰ is CO(R⁹⁶) such as C(═O)C₃-C₆-cycloalkyl, C(═O)O(C₃-C₆-cycloalkyl) or C(═O)NH(C₃-C₆-cycloalkyl), wherein cycloalkyl is cyclopropyl (C₃H₇) or cyclobutyl (C₄H₉).

In a further specific embodiment of formula I, R¹⁰ is CS(R⁹⁶) such as CH(═S).

According to a further specific embodiment of formula I, R¹⁰ is CS(R⁹⁶) such as C(═S)C₁-C₆-alkyl, wherein alkyl is CH₃, C₂H₅, n-propyl, i-propyl, n-butyl, i-butyl, tert-butyl, n-pentyl or i-pentyl.

In a further specific embodiment of formula I, R¹⁰ is CS(R⁹⁶) such as C(═S)C₂-C₆-alkenyl, wherein alkenyl is CH═CH₂, CH₂CH═CH₂.

In a further specific embodiment of formula I, R¹⁰ is CS(R⁹⁶) such as C(═S)C₂-C₆-alkynyl, wherein alkynyl is C≡CH,CH₂C≡CH.

In a further specific embodiment of formula I, R¹⁰ is CS(R⁹⁶) such as C(═S)C₃-C₆-cycloalkyl, wherein cycloalkyl is cyclopropyl (C₃H₇) or cyclobutyl (C₄H₉).

In a further specific embodiment of formula I, R¹⁰ is CS(R⁹⁶) such as C(═S)NHC₁-C₆-alkyl, wherein alkyl is CH₃, C₂H₅, n-propyl, i-propyl, n-butyl, i-butyl, tert-butyl, n-pentyl or i-pentyl.

In still another embodiment of formula I, R⁹ is C₁-C₆-alkoxy, such as OCH₃, OC₂H₅, propoxy, n-butoxy and tert-butoxy, more specifically OCH₃.

In still another embodiment of formula I, R¹⁰ is C₁-C₆-alkoxy, such as OCH₃, OC₂H₅, propoxy, n-butoxy and tert-butoxy, more specifically OCH₃.

In still another embodiment of formula I, R¹⁰ is OR⁹⁵, wherein R⁹⁵ is C₁-C₆-alkoxy, such as OCH₃, OC₂H₅, propoxy, n-butoxy and tert-butoxy, more specifically OCH₃.

In a further specific embodiment of formula I, R¹⁰ is OR⁹⁵, wherein R⁹⁵ C₂-C₆-alkenyl, in particular C₂-C₄-alkenyl, more specifically C₁-C₂-alkenyl. R⁹ is such as OCH═CH₂, OCH₂CH═CH₂.

In a further specific embodiment of formula I, R¹⁰ is OR⁹⁵, wherein R⁹⁵ C₂-C₆-alkynyl, in particular C₂-C₆-alkynyl, in particular C₂-C₄-alkynyl, more specifically C₁-C₂-alkynyl. R⁹ is such as OC≡CH

In still another embodiment of formula I R¹⁰ is OR⁹⁵, wherein R⁹⁵ is C₃-C₆-cycloalkyl, in particular cyclopropyl.

In still another embodiment of formula I, R¹⁰ is OR⁹⁵, wherein R⁹⁵ is C₃-C₆-halocycloalkyl. In a special embodiment R¹ is fully or partially halogenated cyclopropyl.

In still another embodiment of formula I, R¹⁰ is is OR⁹⁵, wherein R⁹⁵ C₃-C₆-cycloalkenyl, in particular cyclopropenyl.

Particularly preferred embodiments of R¹⁰ according to the invention are in Table P14 below, wherein each line of lines P14-1 to P14-40 corresponds to one particular embodiment of the invention, wherein P14-1 to P14-40 are also in any combination with one another a preferred embodiment of the present invention. The connection point to the carbon atom, to which R¹⁰ is bound is marked with “#” in the drawings.

TABLE P14

| No. | R¹⁰ |
|---|---|
| P14-1 | H |
| P14-2 | CH₃ |
| P14-3 | CHF₂ |
| P14-4 | CF₃ |
| P14-5 | C₂H₅ |
| P14-6 | CH₂CH₂CH₃ |
| P14-7 | CH(CH₃)₂ |
| P14-8 | CH₂CH₂CH₂CH₃ |
| P14-9 | CH₂CH(CH₃)₂ |
| P14-10 | C(CH₃)₃ |
| P14-11 | CH₂CH₂CH₂CH₂CH₃ |
| P14-12 | CH₂CH₂CH(CH₃)₂ |
| P14-13 | SO(CH₃)₂ |
| P14-14 | OH |
| P14-15 | OCH₃ |
| P14-16 | OCHF₂ |
| P14-17 | OC₂H₅ |
| P14-18 | CN |
| P14-19 | F |
| P14-20 | Cl |
| P14-21 | Br |
| P14-22 | NO₂ |
| P14-23 | NH₂ |
| P14-24 | CO—NH₂ |
| P14-25 | CO—NH(CH₃) |
| P14-26 | CO—N(CH₃)₂ |
| P14-27 | HNCH₃ |
| P14-28 | HNC₂H₅ |
| P14-29 | C≡CH |
| P14-30 | SO₂ |
| P14-31 | SO₂—CH₃ |
| P14-32 | SO—CH₃ |
| P14-33 | S—CH₃ |
| P14-34 | 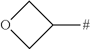 |
| | (C1) |
| P14-35 | 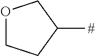 |
| | (C2) |
| P14-36 | 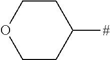 |
| | (C3) |
| P14-37 | 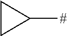 |
| | (C4) |
| P14-38 | 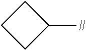 |
| | (C5) |
| P14-39 | 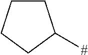 |
| | (C6) |
| P14-40 | 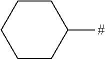 |
| | (C7) |

In still another embodiment of formula I, R⁹ and R¹⁰ together with the carbon atoms to which they are bound form a saturated or partially unsaturated five-, six- or seven-, membered carbocycle, heterocycle or heteroaromatic ring; wherein the heterocycle and heteroaromatic ring contains one, two, three or four heteroatoms selected from N, O and S, wherein the heteroatom N may carry one substituent RN selected from C₁-C₄-alkyl, C₁-C₄-haloalkyl and SO₂Ph, wherein Ph is unsubstituted phenyl or phenyl that is substituted by one, two or three substituents selected from C₁-C₄-alkyl, and wherein the heteroatom S may be in the form of its oxide SO or SO₂; and wherein in each case one or two CH₂ groups of the carbo- or heterocycle may be replaced by a group independently selected from C(═O) and C(═S); and wherein the carbocycle or heterocycle is unsubstituted or substituted by (R¹¹)ₘ, independently selected from halogen, OH, CN, NO₂, SH, NH₂, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₄-alkoxy-C₁-C₄-alkyl, phenyl and phenoxy.

In one embodiment, R⁹ and R¹⁰ form a 3-membered saturated carbocycle. According to one embodiment thereof, the carbocycle is unsubstituted, i.e. it does not carry any substituent R¹¹. In still another embodiment of formula I, it is substituted by R¹¹.

In one embodiment, R⁹ and R¹⁰ form a 4-membered saturated carbocycle. According to one embodiment thereof, the carbocycle is unsubstituted, i.e. it does not carry any substituent R¹¹. In still another embodiment of formula I, it is substituted by R¹¹.

In one embodiment, R⁹ and R¹⁰ form a 5-membered saturated carbocycle. According to one embodiment thereof, the carbocycle is unsubstituted, i.e. it does not carry any substituent R¹¹. In still another embodiment of formula I, it is substituted by R¹¹.

In one embodiment, R⁹ and R¹⁰ form a 6-membered saturated carbocycle. According to one embodiment thereof, the carbocycle is unsubstituted, i.e. it does not carry any substituent R¹¹. In still another embodiment of formula I, it is substituted by R¹¹.

In one embodiment, R⁹ and R¹⁰ form a 7-membered saturated carbocycle. According to one embodiment thereof, the carbocycle is unsubstituted, i.e. it does not carry any substituent R¹¹. In still another embodiment of formula I, it is substituted by R¹¹.

In still another embodiment of formula I,R⁹ and R¹⁰ together with the carbon atoms to which they are bound form a saturated or partially unsaturated five-, six-or seven -membered heterocycle that is unsubstituted or substituted.

In still another embodiment of formula I, the heterocycle formed by R⁹ and R¹⁰ is saturated.

In still another embodiment of formula I, the heterocycle formed by R⁹ and R¹⁰ is a saturated unsubstituted or substituted heterocycle, wherein the heterocycle contains one, two or three, more particularly one or two, specifically one, heteroatom(s) selected from NH, NR^{N}, O, S, S(═O) and S(═O)₂, wherein R″ is defined and preferably defined above. In still another embodiment of formula I, this saturated heterocycle is unsubstituted. In still another embodiment of formula I, the saturated heterocycle is substituted by R¹¹. In one further particular embodiment, said heterocycle is four- or six-membered.

In still another embodiment of formula I, the unsubstituted or substituted and saturated or partially unsaturated heterocycle is three-, four-, five- or six-membered and contains one, two or three, more particularly one or two, heteroatoms selected from NH, NR^{N}, O, S, S(═O) and S(═O)₂, wherein R^{N} is as defined above or preferably selected from C₁-C₂-alkyl, C₁-C₂-haloalkyl and SO₂Ph, wherein Ph is unsubstituted phenyl or phenyl that is substituted by one C₁-C₂-alkyl.

In one further particular embodiment, said heterocycle is four- or six-membered. In still another embodiment of formula I, the heterocycle formed by R⁹ and R¹⁰ contains one, two or three, more specifically one or two, heteroatoms selected from NH and NR^{N}, wherein R^{N} is as defined and preferably defined below, more particularly selected from C₁-C₂-alkyl, C₁-C₂-haloalkyl and SO₂Ph, wherein Ph is unsubstituted phenyl or phenyl that is substituted by one methyl. In one embodiment thereof, it contains one or two heteroatoms NH, in particular one NH. In another embodiment, it contains one or two heteroatoms NR^{N}, in particular one NR^{N}, wherein R^{N} in each case is as defined and preferably defined above.

In still another embodiment of formula I, the heterocycle formed by R⁹ and R¹⁰ contains one, two or three, more specifically one or two, in particular one, heteroatom(s) selected from S, S(═O) and S(═O)₂. In one embodiment thereof, it contains one or two heteroatoms S, in particular one S. In another embodiment, it contains one or two heteroatoms S(═O), in particular one S(═O). In still another embodiment of formula I, it contains one or two heteroatoms S(═O)₂, in particular one S(═O)₂.

In still another embodiment of formula I, the heterocycle formed by R⁹ and R¹⁰contains one or two heteroatoms O. In one embodiment thereof, it contains one heteroatom O. In another embodiment, it contains two heteroatoms O.

In still another embodiment of formula I, the heterocycle formed by R⁹ and R¹⁰ is unsubstituted, i.e. it does not carry any substituent R¹¹. In still another embodiment of formula I, it is substituted by R¹¹.

In one embodiment, R⁹ and R¹⁰ together form a 4-membered saturated heterocycle which contains 1 or 2 heteroatoms, in particular 1 heteroatom, from the group consisting of NH, NR^{N}, O, S, S(═O) and S(═O)₂, as ring members, wherein R^{N} is defined and preferably defined above. In one embodiment, the heterocycle contains one 0 as heteroatom. For example, the formed heterocycle is oxetane. According to one embodiment thereof, the heterocycle is unsubstituted, i.e. it does not carry any substituent R¹¹. In still another embodiment of formula I, it is substituted R¹¹.

In still another embodiment of formula I, R⁹ and R¹⁰ together form a 5-membered saturated heterocycle which contains 1, 2 or 3, in particular 1 or 2, heteroatoms from the group consisting of NH, NR^{N}, O, S, S(═O) and S(═O)₂, as ring members, wherein R^{N} is as defined and preferably defined above. According to one embodiment thereof, the heterocycle is unsubstituted, i.e. it does not carry any substituent R¹¹. In still another embodiment of formula I, it is substituted by R¹¹.

In still another embodiment of formula I, R⁹ and R¹⁰ together form a 6-membered saturated heterocycle which contains 1, 2 or 3, in particular 1 or 2, heteroatoms from the group consisting of NH, NR^{N}, O, S, S(═O) and S(═O)₂, as ring members, wherein R^{N} is as defined and preferably defined below. According to one embodiment thereof, the heterocycle is unsubstituted, i.e. it does not carry any substituent R¹¹. In still another embodiment of formula I, it is substituted by R¹¹. According to one specific embodiment thereof, said 6-membered saturated heterocycle contains 1 or 2 heteroatoms selected from NH and NR^{N}. According to a further specific embodiment thereof, said 6-membered saturated heterocycle contains 1 or 2 heteroatoms O. According to a further specific embodiment thereof, said 6-membered saturated heterocycle contains 1 or 2 heteroatoms selected from S, S(═O) and S(═O)₂. According to one embodiment thereof, the respective 6-membered heterocycle is unsubstituted, i.e. it does not carry any substituent R¹¹. In still another embodiment of formula I, it is substituted by R¹¹.

In still another embodiment of formula I, R⁹ together with R¹⁰ and with the carbon atom to which they are bound form a saturated three-, four-, five-, six- or seven-membered, in particular five- or six-membered carbocycle, that is unsubstituted or is substituted by R¹¹ as defined below. According to one embodiment thereof, R⁹ and R¹⁰ form a cyclopropyl, that is unsubstituted or substituted by R¹¹ as defined below. In still another embodiment thereof, R⁹ and R¹⁰ form a cyclobutyl, that is unsubstituted or is substituted by R¹¹ as defined below. According to still a further embodiment thereof, R⁹ and R¹⁰ form a cyclopentyl, that is unsubstituted or substituted by R¹¹ as defined below. According to still a further embodiment thereof, R⁹ and R¹⁰ form a cyclohexyl, that is unsubstituted or substituted by R¹¹ as defined below. According to still a further embodiment thereof, R⁹ and R¹⁰ form a cycloheptyl, that is unsubstituted or is substituted by R¹¹ as defined below.

R¹¹ are the possible substituents for the carbo- or heterocycle formed by R⁹ and R¹⁰ and is in each case independently selected from halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein

wherein the aliphatic moieties of R¹¹ are unsubstituted or substituted with identical or different groups R¹¹a which independently of one another are selected from:

R^{11a} halogen, OH, CN, C₁-C₆-alkoxy, C₃-C₆-cycloalkyl, C₃-C₆-halocycloalkyl, C₁-C₄-halogenalkoxy, C₁-C₆-alkylthio and phenoxy, wherein the phenyl group is unsubstituted or unsubstituted or substituted with R^{111a} selected from the group consisting of halogen, OH, C₁-C₄-alkyl, C₁-C₄-haloalkyl, C₁-C₄-alkoxy, C₁-C₄-halonalkoxy, CN, C₃-C₆-cycloalkyl, C₃-C₆-halocycloalkyl, C₁-C₄-alkylthio;

wherein the cycloalkyl, heteroaryl and aryl of R¹¹ are unsubstituted or substituted with identical or different groups R^{11b} which independently of one another are selected from:

R^{11b} halogen, OH, CN, C₁-C₄-alkyl, C₁-C₄-alkoxy, C₁-C₄-halogenalkyl, C₃-C₆-cycloalkyl, C₃-C₆-halocycloalkyl, C₁-C₄-haloalkoxy and C₁-C₆-alkylthio.

For every R¹¹ that is present in the inventive compounds, the following embodiments and preferences apply independently of the meaning of any other R¹¹ that may be present in the ring.

In one preferred embodiment, R¹¹ is in each case independently selected from halogen, OH, CN, SH, C₁-C₆-alkyl, C₁-C₆-haloalkyl, C₁-C₆-alkoxy, C₁-C₆-haloalkoxy and C₁-C₆-alkylthio. In one further preferred embodiment, R¹¹ is in each case independently selected from halogen, C₁-C₆-alkyl and C₁-C₆-haloalkyl. In one further particular embodiment, R¹¹ is in each case independently selected from C₁-C₆-alkyl, such as CH₃ and C₂H₅.

According to another embodiment of formula I, R¹¹ is hydrogen.

According to still another embodiment of formula I, R¹¹ is halogen, in particular Br, F or Cl, more specifically F or Cl.

According to still another embodiment of formula I, R¹¹ is OH.

According to still another embodiment of formula I, R¹¹ is CN.

According to still another embodiment of formula I R¹¹ is NH₂, NH(C₁-C₄-alkyl), N(C₁-C₄-alkyl)₂ or NH—SO₂—R^{x}, wherein R^{x} is C₁-C₄-alkyl, C₁-C₄-halogenalkyl, unsubstituted aryl or aryl that is substituted by one, two, three, four or five substituents Rx¹¹ independently selected from C₁-C₄-alkyl.

According to still another embodiment of formula I, R¹¹ is C₁-C₆-alkyl, in particular C₁-C₄-alkyl, such as CH₃.

According to still another embodiment of formula I, R¹¹ is C₁-C₆-haloalkyl, in particular C₁C₄-haloalkyl, such as CF₃, CH F₂, CH₂F, CCl₃, CHCl₂ or CH₂Cl.

According to still another embodiment of formula I, R¹¹ is C₂-C₆-alkenyl or C₂-C₆-haloalkenyl, in particular C₂-C₄-alkenyl or C₂-C₄-haloalkenyl, such as CH═CH₂, C(CH₃)═CH₂, CH₂CH═CH₂.

According to still another embodiment of formula I, R¹¹ is C₂-C₆-alkynyl or C₂-C₆-haloalkynyl, in particular C₂-C₄-alkynyl or C₂-C₄-haloalkynyl, such as C≡CH, CH₂C≡CH.

According to still another embodiment of formula I, R¹¹ is C₁-C₆-alkoxy, in particular C₁-C₄-alkoxy, more specifically C₁-C₂-alkoxy such as OCH₃ or OCH₂CH₃.

According to still another embodiment of formula I, R¹¹ is C₁-C₆-haloalkoxy, in particular C₁-C₄-haloalkoxy, more specifically C₁-C₂-haloalkoxy such as OCF₃, OCHF₂, OCH₂F, OCCl₃, OCHCl₂ or OCH₂Cl, in particular OCF₃, OCHF₂, OCCl₃ or OCHCl₂.

According to still another embodiment of formula I R¹¹ is C₃-C₆-cycloalkyl, in particular cyclopropyl.

According to still another embodiment of formula I, R¹¹ is C₃-C₆-cycloalkyl, for example cyclopropyl, substituted by one, two, three or up to the maximum possible number of identical or different groups R^{11b} as defined and preferably herein.

According to still another embodiment of formula I, R¹¹ is C₃-C₆-halocycloalkyl. In a special embodiment R¹¹ is fully or partially halogenated cyclopropyl.

According to still another embodiment of formula I, R¹¹ is unsubstituted aryl or aryl that is substituted by one, two, three or four R^{11b}, as defined herein. In particular, R¹¹ is unsubstituted phenyl or phenyl that is substituted by one, two, three or four R^{11b}, as defined herein.

According to still another embodiment of formula I, R¹¹ is unsubstituted 5- or 6-membered heteroaryl. According to still a further embodiment, R¹¹ is 5- or 6-membered heteroaryl that is substituted by one, two or three R^{11b}, as defined herein.

R^{N} is the substituent of the heteroatom NR^{N} that is contained in the heterocycle formed by R⁹ and R¹⁰ in some of the inventive compounds. R^{N} is selected from C₁-C₄-alkyl, C₁-C₄-haloalkyl and SO₂Ph, wherein Ph is unsubstituted phenyl or phenyl that is substituted by one, two or three substituents selected from C₁-C₄-alkyl. In one preferred embodiment, R^{N} is in each case independently selected from C₁-C₂-alkyl, C₁-C₂-haloalkyl and SO₂Ph, wherein Ph is unsubstituted phenyl or phenyl that is substituted by one methyl substituents. In one particular embodiment, R^{N} is in each case independently selected from C₁-C₂-alkyl, more particularly methyl. In one particular embodiment, R″ is in each case independently selected from SO₂Ph, wherein Ph is unsubstituted phenyl or phenyl that is substituted by one methyl.

According to still another embodiment of formula I, R¹¹ is in each case independently selected from hydrogen, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy and C₃-C₆-cycloalkyl; wherein the aliphatic moieties of R¹¹ are not further substituted or carry one, two, three, four or five identical or different groups R^{11a} as defined below and wherein the cycloalkyl, heteroaryl and aryl moieties of R¹¹ are not further substituted or carry one, two, three, four or five identical or different groups R^{11b} as defined below.

According to still another embodiment of formula I, R¹¹ is independently selected from hydrogen, halogen, OH, C₁-C₆-alkyl, C₁-C₆-halogenalkyl, C₁-C₆-alkoxy and C₁-C₆-halogenalkoxy, in particular independently selected from F, Cl, Br, CN, OH, C₁-C₄-alkyl, C₁-C₄-halogenalkyl, C₁-C₄-alkoxy and C₁-C₄-halogenalkoxy.

R^{11a} are the possible substituents for the acyclic moieties of R¹¹.

R^{11a} according to the invention is independently selected from halogen, OH, CN, C₁-C₆-alkoxy, C₃-C₆-cycloalkyl, C₃-C₆-halocycloalkyl, C₁-C₄-haloalkoxy, C₁-C₆-alkylthio and phenoxy, wherein the phenyl group is unsubstituted or unsubstituted or substituted with R¹¹¹a selected from the group consisting of halogen, OH, C₁-C₄-alkyl, C₁-C₄-halogenalkyl, C₁-C₄-alkoxy and C₁-C₄-halogenalkoxy, in particular selected from halogen, C₁-C₂-alkyl, C₁-C₂-halogenalkyl, C₁-C₂-alkoxy, C₁-C₂-halogenalkoxy, more specifically selected from halogen, such as F, Cl and Br.

In to one embodiment R^{11a} is independently selected from halogen, OH, CN, C₁-C₂-alkoxy, C₃-C₆-cycloalkyl, C₃-C₆-halocycloalkyl and C₁-C₂-haloalkoxy. Specifically, R^{11a} is independently selected from F, Cl, OH, CN, C₁-C₂-alkoxy, cyclopropyl, 1-F-cyclopropyl, 1-Cl-cyclopropyl and C₁-C₂-halogenalkoxy.

According to one embodiment R^{11a} is independently selected from halogen, such as F, Cl, Br and I, more specifically F, Cl and Br.

According to still another embodiment of formula I, R^{11a} is independently selected from OH, C₃-C₆-cycloalkyl, C₃-C₆-halocycloalkyl and C₁-C₂-haloalkoxy. Specifically, R^{11a} is independently selected from OH, cyclopropyl and C₁-C₂-haloalkoxy.

R^{11b} are the possible substituents for the cycloalkyl, heteroaryl and aryl moieties of R¹¹.

R^{11b} according to the invention is independently selected from halogen, OH, CN, C₁-C₄-alkyl, C₁-C₄-alkoxy, C₁-C₄-haloalkyl, C₃-C₆-cycloalkyl, C₃-C₆-halocycloalkyl and C₁-C₄-haloalkoxy.

According to one embodiment thereof R^{11b} is independently selected from halogen, CN, C₁-C₂-alkyl, C₁-C₂-alkoxy, C₁-C₂-halogalkyl, C₃-C₆-cycloalkyl, C₃-C₆-halocycloalky and C₁-C₂-haloalkoxy. Specifically, R^{11b} is independently selected from F, Cl, OH, CN, CH₃, OCH₃, cyclopropyl, 1-F-cyclopropyl, 1-Cl-cyclopropyl and halogenmethoxy.

According to still another embodiment thereof R^{11b} is independently selected from C₁-C₂-alkyl, C₁-C₂-alkoxy, C₁-C₂-haloalkyl, C₃-C₆-cycloalkyl, C₃-C₆-halocycloalkyl and C₁-C₂-halogenalkoxy. Specifically, R^{11b} is independently selected from OH, CH₃, OCH₃, cyclopropyl, 1-F-cyclopropyl, 1-Cl-cyclopropyl and halomethoxy, more specifically independently selected from OH, CH₃, OCH₃, cyclopropyl, 1-F-cyclopropyl, 1-Cl-cyclopropyl and OCHF₂.

Particularly preferred embodiments of combinations of R⁹ and R¹⁰ according to the invention are in Table P35 below, wherein each line of lines P35-1 to P35-223 corresponds to one particular embodiment of the invention, wherein P35-1 to P35-223 are also in any combination with one another a preferred embodiment of the present invention. The carbon atom, to which R⁹ bound is marked with * in the drawings and the carbon atom, to which R¹⁰ is bound is marked with # in the drawings.

TABLE P35

| line | R⁹ | R¹⁰ |
|---|---|---|
| P35-1 | H | H |
| P35-2 | H | CH₃ |
| P35-3 | H | C₂H₅ |
| P35-4 | H | F |
| P35-5 | H | Cl |
| P35-6 | H | Br |
| P35-7 | H | CN |
| P35-8 | H | OCH₃ |
| P35-9 | H | OCHF₂ |
| P35-10 | H | CHF₂ |
| P35-11 | H | CF₃ |
| P35-12 | H | S—CH₃ |
| P35-13 | H | CH₂F |
| P35-14 | H | C≡CH |
| P35-15 | H | cPr |
| P35-16 | CH₃ | H |
| P35-17 | CH₃ | CH₃ |
| P35-18 | CH₃ | C₂H₅ |
| P35-19 | CH₃ | F |
| P35-20 | CH₃ | Cl |
| P35-21 | CH₃ | Br |
| P35-22 | CH₃ | CN |
| P35-23 | CH₃ | OCH₃ |
| P35-24 | CH₃ | OCHF₂ |
| P35-25 | CH₃ | CHF₂ |
| P35-26 | CH₃ | CF₃ |
| P35-27 | CH₃ | S—CH₃ |
| P35-28 | CH₃ | CH₂F |
| P35-29 | CH₃ | C≡CH |
| P35-30 | CH₃ | cPr |
| P35-31 | C₂H₅ | H |
| P35-32 | C₂H₅ | CH₃ |
| P35-33 | C₂H₅ | C₂H₅ |
| P35-34 | C₂H₅ | F |
| P35-35 | C₂H₅ | Cl |
| P35-36 | C₂H₅ | Br |
| P35-37 | C₂H₅ | CN |
| P35-38 | C₂H₅ | OCH₃ |
| P35-39 | C₂H₅ | OCHF₂ |
| P35-40 | C₂H₅ | CHF₂ |
| P35-41 | C₂H₅ | CF₃ |
| P35-42 | C₂H₅ | S—CH₃ |
| P35-43 | C₂H₅ | CH₂F |
| P35-44 | C₂H₅ | C≡CH |
| P35-45 | C₂H₅ | cPr |
| P35-46 | F | H |
| P35-47 | F | CH₃ |
| P35-48 | F | C₂H₅ |
| P35-49 | F | F |
| P35-50 | F | Cl |
| P35-51 | F | Br |
| P35-52 | F | CN |
| P35-53 | F | OCH₃ |
| P35-54 | F | OCHF₂ |
| P35-55 | F | CHF₂ |
| P35-56 | F | CF₃ |
| P35-57 | F | S—CH₃ |
| P35-58 | F | CH₂F |
| P35-59 | F | C≡CH |
| P35-60 | F | cPr |
| P35-61 | Cl | H |
| P35-62 | Cl | CH₃ |
| P35-63 | Cl | C₂H₅ |
| P35-64 | Cl | F |
| P35-65 | Cl | Cl |
| P35-66 | Cl | Br |
| P35-67 | Cl | CN |
| P35-68 | Cl | OCH₃ |
| P35-69 | Cl | OCHF₂ |
| P35-70 | Cl | CHF₂ |
| P35-71 | Cl | CF₃ |
| P35-72 | Cl | S—CH₃ |
| P35-73 | Cl | CH₂F |
| P35-74 | Cl | C≡CH |
| P35-75 | Cl | cPr |
| P35-76 | Br | H |
| P35-77 | Br | CH₃ |
| P35-78 | Br | C₂H₅ |
| P35-79 | Br | F |
| P35-80 | Br | Cl |
| P35-81 | Br | Br |
| P35-82 | Br | CN |
| P35-83 | Br | OCH₃ |
| P35-84 | Br | OCHF₂ |
| P35-85 | Br | CHF₂ |
| P35-86 | Br | CF₃ |
| P35-87 | Br | S—CH₃ |
| P35-88 | Br | CH₂F |
| P35-89 | Br | C≡CH |
| P35-90 | Br | cPr |
| P35-91 | CN | H |
| P35-92 | CN | CH₃ |
| P35-93 | CN | C₂H₅ |
| P35-94 | CN | F |
| P35-95 | CN | Cl |
| P35-96 | CN | Br |
| P35-97 | CN | CN |
| P35-98 | CN | OCH₃ |
| P35-99 | CN | OCHF₂ |
| P35-100 | CN | CHF₂ |
| P35-101 | CN | CF₃ |
| P35-102 | CN | S—CH₃ |
| P35-103 | OCH₃ | H |
| P35-104 | OCH₃ | CH₂F |
| P35-105 | OCH₃ | C≡CH |
| P35-106 | OCH₃ | cPr |
| P35-107 | OCH₃ | CH₃ |
| P35-108 | OCH₃ | C₂H₅ |
| P35-109 | OCH₃ | F |
| P35-110 | OCH₃ | Cl |
| P35-111 | OCH₃ | Br |
| P35-112 | OCH₃ | CN |
| P35-113 | OCH₃ | OCH₃ |
| P35-114 | OCH₃ | OCHF₂ |
| P35-115 | OCH₃ | CHF₂ |
| P35-116 | OCH₃ | CF₃ |
| P35-117 | OCH₃ | S—CH₃ |
| P35-118 | OCH₃ | CH₂F |
| P35-119 | OCH₃ | C≡CH |
| P35-120 | OCH₃ | cPr |
| P35-121 | OCHF₂ | H |
| P35-122 | OCHF₂ | CH₃ |
| P35-123 | OCHF₂ | C₂H₅ |
| P35-124 | OCHF₂ | F |
| P35-125 | OCHF₂ | Cl |
| P35-126 | OCHF₂ | Br |
| P35-127 | OCHF₂ | CN |
| P35-128 | OCHF₂ | OCH₃ |
| P35-129 | OCHF₂ | OCHF₂ |
| P35-130 | OCHF₂ | CHF₂ |
| P35-131 | OCHF₂ | CF₃ |
| P35-132 | OCHF₂ | S—CH₃ |
| P35-133 | OCHF₂ | CH₂F |
| P35-134 | OCHF₂ | C≡CH |
| P35-135 | OCHF₂ | cPr |
| P35-136 | CHF₂ | H |
| P35-137 | CHF₂ | CH₃ |
| P35-138 | CHF₂ | C₂H₅ |
| P35-139 | CHF₂ | F |
| P35-140 | CHF₂ | Cl |
| P35-141 | CHF₂ | Br |
| P35-142 | CHF₂ | CN |
| P35-143 | CHF₂ | OCH₃ |
| P35-144 | CHF₂ | OCHF₂ |
| P35-145 | CHF₂ | CHF₂ |
| P35-146 | CHF₂ | CF₃ |
| P35-147 | CHF₂ | S—CH₃ |
| P35-148 | CHF₂ | CH₂F |
| P35-149 | CHF₂ | C≡CH |
| P35-150 | CHF₂ | cPr |
| P35-151 | CF₃ | H |
| P35-152 | CF₃ | CH₃ |
| P35-153 | CF₃ | C₂H₅ |
| P35-154 | CF₃ | F |
| P35-155 | CF₃ | Cl |
| P35-156 | CF₃ | Br |
| P35-157 | CF₃ | CN |
| P35-158 | CF₃ | OCH₃ |
| P35-159 | CF₃ | OCHF₂ |
| P35-160 | CF₃ | CHF₂ |
| P35-161 | CF₃ | CF₃ |
| P35-162 | CF₃ | S—CH₃ |
| P35-163 | CF₃ | CH₂F |
| P35-164 | CF₃ | C≡CH |
| P35-165 | CF₃ | cPr |
| P35-166 | CH₂F | H |
| P35-167 | CH₂F | CH₃ |
| P35-168 | CH₂F | C₂H₅ |
| P35-169 | CH₂F | F |
| P35-170 | CH₂F | Cl |
| P35-171 | CH₂F | Br |
| P35-172 | CH₂F | CN |
| P35-173 | CH₂F | OCH₃ |
| P35-174 | CH₂F | OCHF₂ |
| P35-175 | CH₂F | CHF₂ |
| P35-176 | CH₂F | CF₃ |
| P35-177 | CH₂F | S—CH₃ |
| P35-178 | CH₂F | CH₂F |
| P35-179 | CH₂F | C≡CH |
| P35-180 | CH₂F | cPr |
| P35-181 | C≡CH | H |
| P35-182 | C≡CH | CH₃ |
| P35-183 | C≡CH | C₂H₅ |
| P35-184 | C≡CH | F |
| P35-185 | C≡CH | Cl |
| P35-186 | C≡CH | Br |
| P35-187 | C≡CH | CN |
| P35-188 | C≡CH | OCH₃ |
| P35-189 | C≡CH | OCHF₂ |
| P35-190 | C≡CH | CHF₂ |
| P35-191 | C≡CH | CF₃ |
| P35-192 | C≡CH | S—CH₃ |
| P35-193 | C≡CH | CH₂F |
| P35-194 | C≡CH | C≡CH |
| P35-195 | C≡CH | cPr |
| P35-196 | cPr | H |
| P35-197 | cPr | CH₃ |
| P35-198 | cPr | C₂H₅ |
| P35-199 | cPr | F |
| P35-200 | cPr | Cl |
| P35-201 | cPr | Br |
| P35-202 | cPr | CN |
| P35-203 | cPr | OCH₃ |
| P35-204 | cPr | OCHF₂ |
| P35-205 | cPr | CHF₂ |
| P35-206 | cPr | CF₃ |
| P35-207 | cPr | S—CH₃ |
| P35-208 | cPr | CH₂F |
| P35-209 | cPr | C≡CH |
| P35-210 | cPr | cPr |

| | |
|---|---|
| P35-211 | 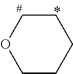 |
| P35-212 | 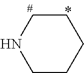 |
| P35-213 | 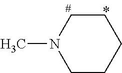 |
| P35-214 | 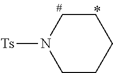 |
| P35-215 | 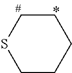 |
| P35-216 | 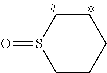 |
| P35-217 | 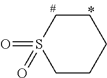 |
| P35-218 | 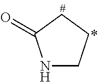 |
| P35-219 | 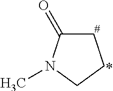 |
| P35-220 | 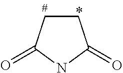 |
| P35-221 | 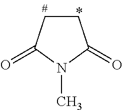 |
| P35-222 | 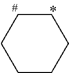 |
| P35-223 | 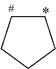 |

Another prefer embodiment are following structures:

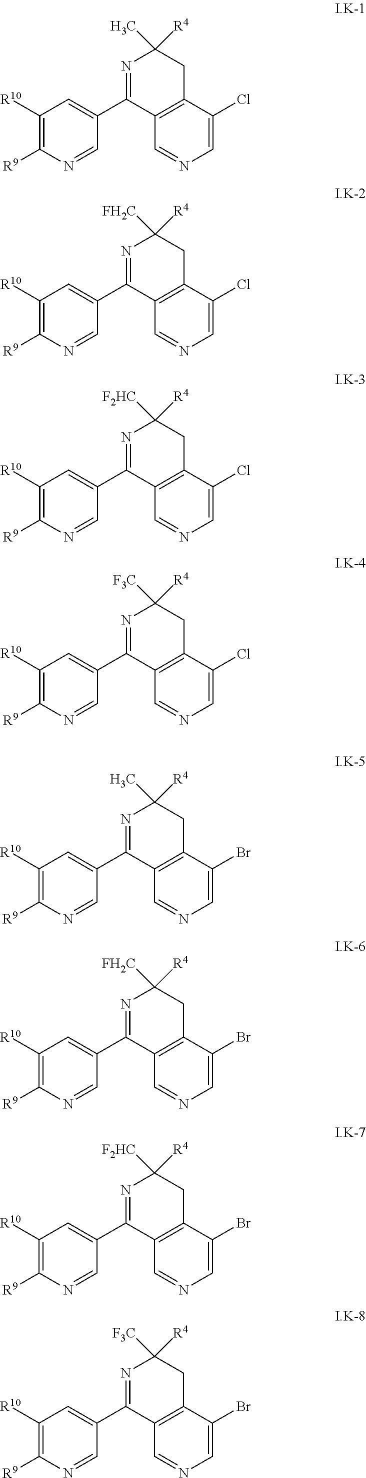

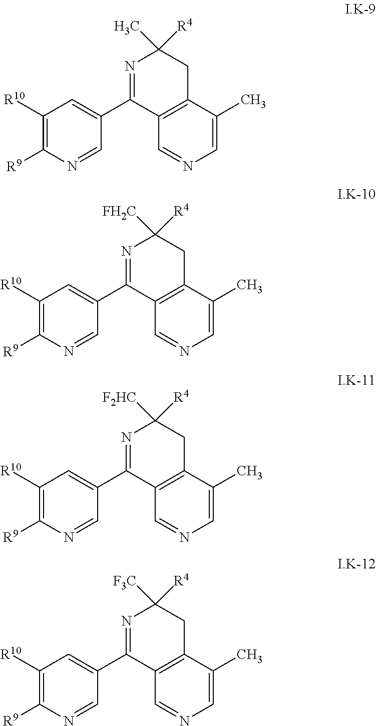

In particular with a view to their use, according to one embodiment, preference is given to the compounds of the formulae I.K-1, I.K-2, I.K-3, I.K-4, I.K-5, I.K-6, I.K-7, I.K-8, I.K-9, I.K-10, I.K-11 and I.K-12 that are compiled in the Tables 1-1 to 1-7, Tables 2-1 to 2-7, Tables 3-1 to 3-7, Tables 4-1 to 4-7, Tables 5-1 to 5-7, Tables 6-1 to 6-7, Tables 7-1 to 7-7, Tables 8-1 to 8-7, Tables 9-1 to 9-7, Tables 10-1 to 10-7, Tables 11-1 to 11-7 and Tables 12-1 to 12-7. Each of the groups mentioned for a substituent in the tables is furthermore per se, independently of the combination in which it is mentioned, a particularly preferred aspect of the substituent in question.

Table 1-1 Compounds of the formula I.K-1 in which R⁹ is CH₃, R¹⁰ is CH₃ and the meaning of R⁴for each individual compound corresponds in each case to one line of Table A (compounds I.K-1.1-1.A-1 to I.K-1.1-1.A-35).

Table 1-2 Compounds of the formula I.K-1 in which R⁹ is CHF₂, R¹⁰ is CH₃ and the meaning of R⁴for each individual compound corresponds in each case to one line of Table A (compounds I.K-1.1-2.A-1 to I.K-1.1-2.A-35).

Table 1-3 Compounds of the formula I.K-1 in which R⁹ is CH₃, R¹⁰ is C≡CH and the meaning of R⁴for each individual compound corresponds in each case to one line of Table A (compounds I.K-1.1-3.A-1 to I.K-1.1-3.A-35).

Table 1-4 Compounds of the formula I.K-1 in which R⁹ is CH₂F, R¹⁰ is CH₃ and the meaning of R⁴ for each individual compound corresponds in each case to one line of Table A (compounds I.K-1.1-4.A-1 to I.K-1.1-4.A-35).

Table 1-5 Compounds of the formula I.K-1 in which R⁹ is CHF₂, R¹⁰ is Br and the meaning of R⁴ for each individual compound corresponds in each case to one line of Table A (compounds I.K-1.1-5.A-1 to I.K-1.1-5.A-35).

Table 1-6 Compounds of the formula I.K-1 in which R⁹ is CH₃, R¹⁰ is CH₂F and the meaning of R⁴for each individual compound corresponds in each case to one line of Table A (compounds I.K-1.1-6.A-1 to I.K-1.1-6.A-35).

Table 1-7 Compounds of the formula I.K-1 in which R⁹ is CHF₂, R¹⁰ is OCH₃ and the meaning of R⁴for each individual compound corresponds in each case to one line of Table A (compounds I.K-1.1-7.A-1 to I.K-1.1-7.A-35).

Table 2-1 Compounds of the formula I.K-2 in which R⁹ is CH₃, R¹⁰ is CH₃ and the meaning of R⁴for each individual compound corresponds in each case to one line of Table A (compounds I.K-2.2-1.A-1 to I.K-2.2-1.A-35).

Table 2-2 Compounds of the formula I.K-2 in which R⁹ is CHF₂, R¹⁰ is CH₃ and the meaning of R⁴for each individual compound corresponds in each case to one line of Table A (compounds I.K-2.2-2.A-1 to I.K-2.2-2.A-35).

Table 2-3 Compounds of the formula I.K-2 in which R⁹ is CH₃, R¹⁰ is C≡CH and the meaning of R⁴for each individual compound corresponds in each case to one line of Table A (compounds I.K-2.2-3.A-1 to I.K-2.2-3.A-35).

Table 2-4 Compounds of the formula I.K-2 in which R⁹ is CH₂F, R¹⁰ is CH₃ and the meaning of R⁴for each individual compound corresponds in each case to one line of Table A (compounds I.K-2.2-4.A-1 to I.K-2.2-4.A-35).

Table 2-5 Compounds of the formula I.K-2 in which R⁹ is CHF₂, R¹⁰ is Br and the meaning of R⁴ for each individual compound corresponds in each case to one line of Table A (compounds I.K-2.2-5.A-1 to I.K-2.2-5.A-35).

Table 2-6 Compounds of the formula I.K-2 in which R⁹ is CH₃, R¹⁰ is CH₂F and the meaning of R⁴for each individual compound corresponds in each case to one line of Table A (compounds I.K-2.2-6.A-1 to I.K-2.2-6.A-35).

Table 2-7 Compounds of the formula I.K-2 in which R⁹ is CHF₂, R¹⁰ is OCH₃ and the meaning of R⁴for each individual compound corresponds in each case to one line of Table A (compounds I.K-2.2-7.A-1 to I.K-2.2-7.A-35).

Table 3-1 Compounds of the formula I.K-3 in which R⁹ is CH₃, R¹⁰ is CH₃ and the meaning of R⁴for each individual compound corresponds in each case to one line of Table A (compounds I.K-3.3-1.A-1 to I.K-3.3-1.A-35).

Table 3-2 Compounds of the formula I.K-3 in which R⁹ is CHF₂, R¹⁰ is CH₃ and the meaning of R⁴for each individual compound corresponds in each case to one line of Table A (compounds I.K-3.3-2.A-1 to I.K-3.3-2.A-35).

Table 3-3 Compounds of the formula I.K-3 in which R⁹ is CH₃, R¹⁰ is C≡CH and the meaning of R⁴for each individual compound corresponds in each case to one line of Table A (compounds I.K-3.3-3.A-1 to I.K-3.3-3.A-35).

Table 3-4 Compounds of the formula I.K-3 in which R⁹ is CH₂F, R¹⁰ is CH₃ and the meaning of R⁴for each individual compound corresponds in each case to one line of Table A (compounds I.K-3.3-4.A-1 to I.K-3.3-4.A-35).

Table 3-5 Compounds of the formula I.K-3 in which R⁹ is CHF₂, R¹⁰ is Br and the meaning of R⁴ for each individual compound corresponds in each case to one line of Table A (compounds I.K-3.3-5.A-1 to I.K-3.3-5.A-35).

Table 3-6 Compounds of the formula I.K-3 in which R⁹ is CH₃, R¹⁰ is CH₂F and the meaning of R⁴for each individual compound corresponds in each case to one line of Table A (compounds I.K-3.3-6.A-1 to I.K-3.3-6.A-35).

Table 3-7 Compounds of the formula I.K-3 in which R⁹ is CHF₂, R¹⁰ is OCH₃ and the meaning of R⁴for each individual compound corresponds in each case to one line of Table A (compounds I.K-3.3-7.A-1 to I.K-3.3-7.A-35).

Table 4-1 Compounds of the formula I.K-4 in which R⁹ is CH₃, R¹⁰ is CH₃ and the meaning of R⁴for each individual compound corresponds in each case to one line of Table A (compounds I.K-4.4-1.A-1 to I.K-4.4-1.A-35).

Table 4-2 Compounds of the formula I.K-4 in which R⁹ is CHF₂, R¹⁰ is CH₃ and the meaning of R⁴for each individual compound corresponds in each case to one line of Table A (compounds I.K-4.4-2.A-1 to I.K-4.4-2.A-35).

Table 4-3 Compounds of the formula I.K-4 in which R⁹ is CH₃, R¹⁰ is C≡CH and the meaning of R⁴for each individual compound corresponds in each case to one line of Table A (compounds I.K-4.4-3.A-1 to I.K-4.4-3.A-35).

Table 4-4 Compounds of the formula I.K-4 in which R⁹ is CH₂F, R¹⁰ is CH₃ and the meaning of R⁴for each individual compound corresponds in each case to one line of Table A (compounds I.K-4.4-4.A-1 to I.K-4.4-4.A-35).

Table 4-5 Compounds of the formula I.K-4 in which R⁹ is CHF₂, R¹⁰ is Br and the meaning of R⁴ for each individual compound corresponds in each case to one line of Table A (compounds I.K-4.4-5.A-1 to I.K-4.4-5.A-35).

Table 4-6 Compounds of the formula I.K-4 in which R⁹ is CH₃, R¹⁰ is CH₂F and the meaning of

⁴for each individual compound corresponds in each case to one line of Table A (compounds I.K-4.4-6.A-1 to I.K-4.4-6.A-35).

Table 4-7 Compounds of the formula I.K-4 in which R⁹ is CHF₂, R¹⁰ is OCH₃ and the meaning of R⁴for each individual compound corresponds in each case to one line of Table A (compounds I.K-4.4-7.A-1 to I.K-4.4-7.A-35).

Table 5-1 Compounds of the formula I.K-5 in which R⁹ is CH₃, R¹⁰ is CH₃ and the meaning of R⁴for each individual compound corresponds in each case to one line of Table A (compounds I.K-5.5-1.A-1 to I.K-5.5-1.A-35).

Table 5-2 Compounds of the formula I.K-5 in which R⁹ is CHF₂, R¹⁰ is CH₃ and the meaning of R⁴for each individual compound corresponds in each case to one line of Table A (compounds I.K-5.5-2.A-1 to I.K-5.5-2.A-35).

Table 5-3 Compounds of the formula I.K-5 in which R⁹ is CH₃, R¹⁰ is C≡CH and the meaning of R⁴for each individual compound corresponds in each case to one line of Table A (compounds I.K-5.5-3.A-1 to I.K-5.5-3.A-35).

Table 5-4 Compounds of the formula I.K-5 in which R⁹ is CH₂F, R¹⁰ is CH₃ and the meaning of R⁴for each individual compound corresponds in each case to one line of Table A (compounds I.K-5.5-4.A-1 to I.K-5.5-4.A-35).

Table 5-5 Compounds of the formula I.K-5 in which R⁹ is CHF₂, R¹⁰ is Br and the meaning of R⁴ for each individual compound corresponds in each case to one line of Table A (compounds I.K-5.5-5.A-1 to I.K-5.5-5.A-35).

Table 5-6 Compounds of the formula I.K-5 in which R⁹ is CH₃, R¹⁰ is CH₂F and the meaning of R⁴for each individual compound corresponds in each case to one line of Table A (compounds I.K-5.5-6.A-1 to I.K-5.5-6.A-35).

Table 5-7 Compounds of the formula I.K-5 in which R⁹ is CHF₂, R¹⁰ is OCH₃ and the meaning of R⁴for each individual compound corresponds in each case to one line of Table A (compounds I.K-5.5-7.A-1 to I.K-5.5-7.A-35).

Table 6-1 Compounds of the formula I.K-6 in which R⁹ is CH₃, R¹⁰ is CH₃ and the meaning of R⁴for each individual compound corresponds in each case to one line of Table A (compounds I.K-6.6-1.A-1 to I.K-6.6-1.A-35).

Table 6-2 Compounds of the formula I.K-6 in which R⁹ is CHF₂, R¹⁰ is CH₃ and the meaning of R⁴for each individual compound corresponds in each case to one line of Table A (compounds I.K-6.6-2.A-1 to I.K-6.6-2.A-35).

Table 6-3 Compounds of the formula I.K-6 in which R⁹ is CH₃, R¹⁰ is C≡CH and the meaning of R⁴for each individual compound corresponds in each case to one line of Table A (compounds I.K-6.6-3.A-1 to I.K-6.6-3.A-35).

Table 6-4 Compounds of the formula I.K-6 in which R⁹ is CH₂F, R¹⁰ is CH₃ and the meaning of R⁴for each individual compound corresponds in each case to one line of Table A (compounds I.K-6.6-4.A-1 to I.K-6.6-4.A-35).

Table 6-5 Compounds of the formula I.K-6 in which R⁹ is CHF₂, R¹⁰ is Br and the meaning of R⁴ for each individual compound corresponds in each case to one line of Table A (compounds I.K-6.6-5.A-1 to I.K-6.6-5.A-35).

Table 6-6 Compounds of the formula I.K-6 in which R⁹ is CH₃, R¹⁰ is CH₂F and the meaning of R⁴for each individual compound corresponds in each case to one line of Table A (compounds I.K-6.6-6.A-1 to I.K-6.6-6.A-35).

Table 6-7 Compounds of the formula I.K-6 in which R⁹ is CHF₂, R¹⁰ is OCH₃ and the meaning of R⁴for each individual compound corresponds in each case to one line of Table A (compounds I.K-6.6-7.A-1 to I.K-6.6-7.A-35).

Table 7-1 Compounds of the formula I.K-7 in which R⁹ is CH₃, R¹⁰ is CH₃ and the meaning of R⁴for each individual compound corresponds in each case to one line of Table A (compounds I.K-7.7-1.A-1 to I.K-7.7-1.A-35).

Table 7-2 Compounds of the formula I.K-7 in which R⁹ is CHF₂, R¹⁰ is CH₃ and the meaning of R⁴for each individual compound corresponds in each case to one line of Table A (compounds I.K-7.7-2.A-1 to I.K-7.7-2.A-35).

Table 7-3 Compounds of the formula I.K-7 in which R⁹ is CH₃, R¹⁰ is C≡CH and the meaning of R⁴for each individual compound corresponds in each case to one line of Table A (compounds I.K-7.7-3.A-1 to I.K-7.7-3.A-35).

Table 7-4 Compounds of the formula I.K-7 in which R⁹ is CH₂F, R¹⁰ is CH₃ and the meaning of R⁴for each individual compound corresponds in each case to one line of Table A (compounds I.K-7.7-4.A-1 to I.K-7.7-4.A-35).

Table 7-5 Compounds of the formula I.K-7 in which R⁹ is CHF₂, R¹⁰ is Br and the meaning of R⁴ for each individual compound corresponds in each case to one line of Table A (compounds I.K-7.7-5.A-1 to I.K-7.7-5.A-35).

Table 7-6 Compounds of the formula I.K-7 in which R⁹ is CH₃, R¹⁰ is CH₂F and the meaning of R⁴for each individual compound corresponds in each case to one line of Table A (compounds I.K-7.7-6.A-1 to I.K-7.7-6.A-35).

Table 7-7 Compounds of the formula I.K-7 in which R⁹ is CHF₂, R¹⁰ is OCH₃ and the meaning of R⁴for each individual compound corresponds in each case to one line of Table A (compounds I.K-7.7-7.A-1 to I.K-7.7-7.A-35).

Table 8-1 Compounds of the formula I.K-8 in which R⁹ is CH₃, R¹⁰ is CH₃ and the meaning of R⁴for each individual compound corresponds in each case to one line of Table A (compounds I.K-8.8-1.A-1 to I.K-8.8-1.A-35).

Table 8-2 Compounds of the formula I.K-8 in which R⁹ is CHF₂, R¹⁰ is CH₃ and the meaning of R⁴for each individual compound corresponds in each case to one line of Table A (compounds I.K-8.8-2.A-1 to I.K-8.8-2.A-35).

Table 8-3 Compounds of the formula I.K-8 in which R⁹ is CH₃, R¹⁰ is C≡CH and the meaning of R⁴for each individual compound corresponds in each case to one line of Table A (compounds I.K-8.8-3.A-1 to I.K-8.8-3.A-35).

Table 8-4 Compounds of the formula I.K-8 in which R⁹ is CH₂F, R¹⁰ is CH₃ and the meaning of R⁴for each individual compound corresponds in each case to one line of Table A (compounds I.K-8.8-4.A-1 to I.K-8.8-4.A-35).

Table 8-5 Compounds of the formula I.K-8 in which R⁹ is CHF₂, R¹⁰ is Br and the meaning of R⁴ for each individual compound corresponds in each case to one line of Table A (compounds I.K-8.8-5.A-1 to I.K-8.8-5.A-35).

Table 8-6 Compounds of the formula I.K-8 in which R⁹ is CH₃, R¹⁰ is CH₂F and the meaning of R⁴for each individual compound corresponds in each case to one line of Table A (compounds I.K-8.8-6.A-1 to I.K-8.8-6.A-35).

Table 8-7 Compounds of the formula I.K-8 in which R⁹ is CHF₂, R¹⁰ is OCH₃ and the meaning of R⁴for each individual compound corresponds in each case to one line of Table A (compounds I.K-8.8-7.A-1 to I.K-8.8-7.A-35).

Table 9-1 Compounds of the formula I.K-9 in which R⁹ is CH₃, R¹⁰ is CH₃ and the meaning of R⁴for each individual compound corresponds in each case to one line of Table A (compounds I.K-9.9-1.A-1 to I.K-9.9-1.A-35).

Table 9-2 Compounds of the formula I.K-9 in which R⁹ is CHF₂, R¹⁰ is CH₃ and the meaning of R⁴for each individual compound corresponds in each case to one line of Table A (compounds I.K-9.9-2.A-1 to I.K-9.9-2.A-35).

Table 9-3 Compounds of the formula I.K-9 in which R⁹ is CH₃, R¹⁰ is C≡CH and the meaning of R⁴for each individual compound corresponds in each case to one line of Table A (compounds I.K-9.9-3.A-1 to I.K-9.9-3.A-35).

Table 9-4 Compounds of the formula I.K-9 in which R⁹ is CH₂F, R¹⁰ is CH₃ and the meaning of R⁴for each individual compound corresponds in each case to one line of Table A (compounds I.K-9.9-4.A-1 to I.K-9.9-4.A-35).

Table 9-5 Compounds of the formula I.K-9 in which R⁹ is CHF₂, R¹⁰ is Br and the meaning of R⁴ for each individual compound corresponds in each case to one line of Table A (compounds I.K-9.9-5.A-1 to I.K-9.9-5.A-35).

Table 9-6 Compounds of the formula I.K-9 in which R⁹ is CH₃, R¹⁰ is CH₂F and the meaning of R⁴for each individual compound corresponds in each case to one line of Table A (compounds I.K-9.9-6.A-1 to I.K-9.9-6.A-35).

Table 9-7 Compounds of the formula I.K-9 in which R⁹ is CHF₂, R¹⁰ is OCH₃ and the meaning of R⁴for each individual compound corresponds in each case to one line of Table A (compounds I.K-9.9-7.A-1 to I.K-9.9-7.A-35).

Table 10-1 Compounds of the formula I.K-10 in which R⁹ is CH₃, R¹⁰ is CH₃ and the meaning of R⁴for each individual compound corresponds in each case to one line of Table A (compounds I.K-10.10-1.A-1 to I.K-10.10-1.A-35).

Table 10-2 Compounds of the formula I.K-10 in which R⁹ is CHF₂, R¹⁰ is CH₃ and the meaning of R⁴for each individual compound corresponds in each case to one line of Table A (compounds I.K-10.10-2.A-1 to I.K-10.10-2.A-35).

Table 10-3 Compounds of the formula I.K-10 in which R⁹ is CH₃, R¹⁰ is C≡CH and the meaning of R⁴for each individual compound corresponds in each case to one line of Table A (compounds I.K-10.10-3.A-1 to I.K-10.10-3.A-35).

Table 10-4 Compounds of the formula I.K-10 in which R⁹ is CH₂F, R¹⁰ is CH₃ and the meaning of R⁴for each individual compound corresponds in each case to one line of Table A (compounds I.K-10.10-4.A-1 to I.K-10.10-4.A-35).

Table 10-5 Compounds of the formula I.K-10 in which R⁹ is CHF₂, R¹⁰ is Br and the meaning of R⁴ for each individual compound corresponds in each case to one line of Table A (compounds I.K-10.10-5.A-1 to I.K-10.10-5.A-35).

Table 10-6 Compounds of the formula I.K-10 in which R⁹ is CH₃, R¹⁰ is CH₂F and the meaning of R⁴for each individual compound corresponds in each case to one line of Table A (compounds I.K-10.10-6.A-1 to I.K-10.10-6.A-35).

Table 10-7 Compounds of the formula I.K-10 in which R⁹ is CHF₂, R¹⁰ is OCH₃ and the meaning of R⁴for each individual compound corresponds in each case to one line of Table A (compounds I.K-10.10-7.A-1 to I.K-10.10-7.A-35).

Table 11-1 Compounds of the formula I.K-11 in which R⁹ is CH₃, R¹⁰ is CH₃ and the meaning of R⁴for each individual compound corresponds in each case to one line of Table A (compounds I.K-11.11-1.A-1 to I.K-11.11-1.A-35).

Table 11-2 Compounds of the formula I.K-11 in which R⁹ is CHF₂, R¹⁰ is CH₃ and the meaning of R⁴for each individual compound corresponds in each case to one line of Table A (compounds I.K-11.11-2.A-1 to I.K-11.11-2.A-35).

Table 11-3 Compounds of the formula I.K-11 in which R⁹ is CH₃, R¹⁰ is C≡CH and the meaning of R⁴for each individual compound corresponds in each case to one line of Table A (compounds I.K-11.11-3.A-1 to I.K-11.11-3.A-35).

Table 11-4 Compounds of the formula I.K-11 in which R⁹ is CH₂F, R¹⁰ is CH₃ and the meaning of R⁴for each individual compound corresponds in each case to one line of Table A (compounds I.K-11.11-4.A-1 to I.K-11.11-4.A-35).

Table 11-5 Compounds of the formula I.K-11 in which R⁹ is CHF₂, R¹⁰ is Br and the meaning of R⁴ for each individual compound corresponds in each case to one line of Table A (compounds I.K-11.11-5.A-1 to I.K-11.11-5.A-35).

Table 11-6 Compounds of the formula I.K-11 in which R⁹ is CH₃, R¹⁰ is CH₂F and the meaning of R⁴for each individual compound corresponds in each case to one line of Table A (compounds I.K-11.11-6.A-1 to I.K-11.11-6.A-35).

Table 11-7 Compounds of the formula I.K-11 in which R⁹ is CHF₂, R¹⁰ is OCH₃ and the meaning of R⁴for each individual compound corresponds in each case to one line of Table A (compounds I.K-11.11-7.A-1 to I.K-11.11-7.A-35).

Table 12-1 Compounds of the formula I.K-12 in which R⁹ is CH₃, R¹⁰ is CH₃ and the meaning of R⁴for each individual compound corresponds in each case to one line of Table A (compounds I.K-12.12-1.A-1 to I.K-12.12-1.A-35).

Table 12-2 Compounds of the formula I.K-12 in which R⁹ is CHF₂, R¹⁰ is CH₃ and the meaning of R⁴for each individual compound corresponds in each case to one line of Table A (compounds I.K-12.12-2.A-1 to I.K-12.12-2.A-35).

Table 12-3 Compounds of the formula I.K-12 in which R⁹ is CH₃, R¹⁰ is C≡CH and the meaning of R⁴for each individual compound corresponds in each case to one line of Table A (compounds I.K-12.12-3.A-1 to I.K-12.12-3.A-35).

Table 12-4 Compounds of the formula I.K-12 in which R⁹ is CH₂F, R¹⁰ is CH₃ and the meaning of R⁴for each individual compound corresponds in each case to one line of Table A (compounds I.K-12.12-4.A-1 to I.K-12.12-4.A-35).

Table 12-5 Compounds of the formula I.K-12 in which R⁹ is CHF₂, R¹⁰ is Br and the meaning of R⁴ for each individual compound corresponds in each case to one line of Table A (compounds I.K-12.12-5.A-1 to I.K-12.12-5.A-35).

Table 12-6 Compounds of the formula I.K-12 in which R⁹ is CH₃, R¹⁰ is CH₂F and the meaning of R⁴for each individual compound corresponds in each case to one line of Table A (compounds I.K-12.12-6.A-1 to I.K-12.12-6.A-35).

Table 12-7 Compounds of the formula I.K-12 in which R⁹ is CHF₂, R¹⁰ is OCH₃ and the meaning of R⁴for each individual compound corresponds in each case to one line of Table A (compounds I.K-12.12-7.A-1 to I.K-12.12-7.A-35).

Another prefer embodiments are following intermediates X:

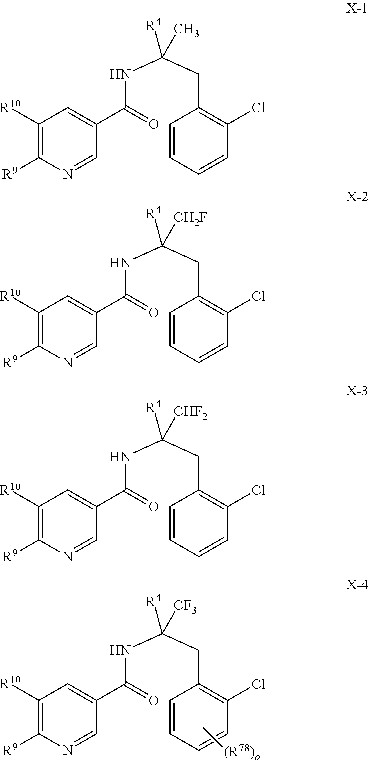

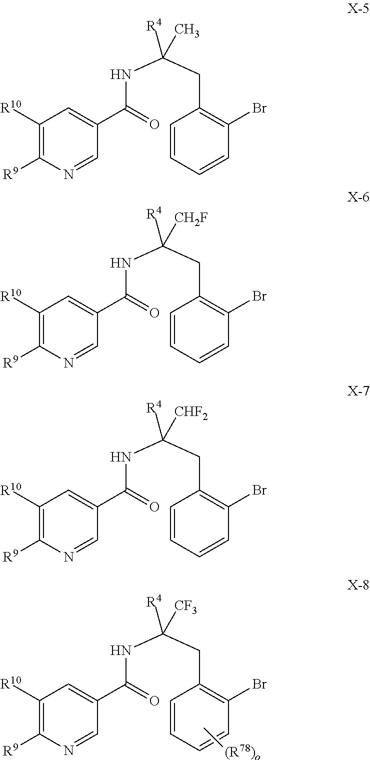

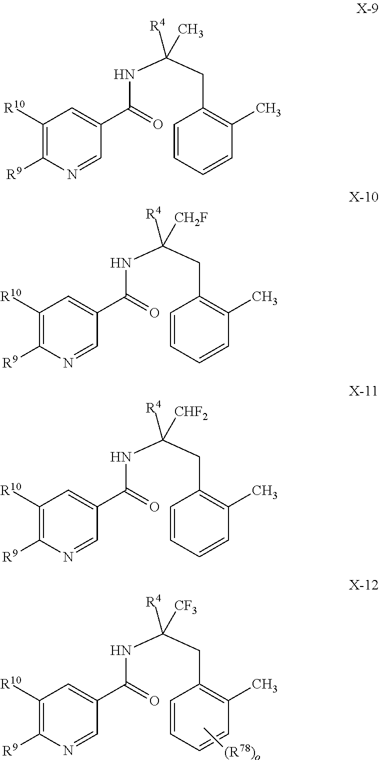

In particular with a view to their use, according to one embodiment, preference is given to the compounds of the formulae X-1, X-2, X-3, X-4, X-5, X-6, X-7, X-8, X-9, X-10, X-11 and X-12 that are compiled in the Tables 1-1 to 1-7, Tables 2-1 to 2-7, Tables 3-1 to 3-7, Tables 4-1 to 4-7, Tables 5-1 to 5-7, Tables 6-1 to 6-7, Tables 7-1 to 7-7, Tables 8-1 to 8-7, Tables 9-1 to 9-7, Tables 10-1 to 10-7, Tables 11-1 to 11-7 and Tables 12-1 to 12-7. Each of the groups mentioned for a substituent in the tables is furthermore per se, independently of the combination in which it is mentioned, a particularly preferred aspect of the substituent in question.

Table 1-1 Compounds of the formula X-1 in which R⁹ is CH₃, R¹⁰ is CH₃ and the meaning of R⁴for each individual compound corresponds in each case to one line of Table A (compounds X-1.1-1.A-1 to X-1.1-1.A-35).

Table 1-2 Compounds of the formula X-1 in which R⁹ is CHF₂, R¹⁰ is CH₃ and the meaning of R⁴for each individual compound corresponds in each case to one line of Table A (compounds X-1.1-2.A-1 to X-1.1-2.A-35).

Table 1-3 Compounds of the formula X-1 in which R⁹ is CH₃, R¹⁰ is C≡CH and the meaning of R⁴for each individual compound corresponds in each case to one line of Table A (compounds X-1.1-3.A-1 to X-1.1-3.A-35).

Table 1-4 Compounds of the formula X-1 in which R⁹ is CH₂F, R¹⁰ is CH₃ and the meaning of R⁴for each individual compound corresponds in each case to one line of Table A (compounds X-1.1-4.A-1 to X-1.1-4.A-35).

Table 1-5 Compounds of the formula X-1 in which R⁹ is CHF₂, R¹⁰ is Br and the meaning of R⁴ for each individual compound corresponds in each case to one line of Table A (compounds X-1.1-5.A-1 to X-1.1-5.A-35).

Table 1-6 Compounds of the formula X-1 in which R⁹ is CH₃, R¹⁰ is CH₂F and the meaning of R⁴for each individual compound corresponds in each case to one line of Table A (compounds X-1.1-6.A-1 to X-1.1-6.A-35).

Table 1-7 Compounds of the formula X-1 in which R⁹ is CHF₂, R¹⁰ is OCH₃ and the meaning of R⁴for each individual compound corresponds in each case to one line of Table A (compounds X-1.1-7.A-1 to X-1.1-7.A-35).

Table 2-1 Compounds of the formula X-2 in which R⁹ is CH₃, R¹⁰ is CH₃ and the meaning of R⁴for each individual compound corresponds in each case to one line of Table A (compounds X-2.2-1.A-1 to X-2.2-1.A-35).

Table 2-2 Compounds of the formula X-2 in which R⁹ is CHF₂, R¹⁰ is CH₃ and the meaning of R⁴for each individual compound corresponds in each case to one line of Table A (compounds X-2.2-2.A-1 to X-2.2-2.A-35).

Table 2-3 Compounds of the formula X-2 in which R⁹ is CH₃, R¹⁰ is C≡CH and the meaning of R⁴for each individual compound corresponds in each case to one line of Table A (compounds X-2.2-3.A-1 to X-2.2-3.A-35).

Table 2-4 Compounds of the formula X-2 in which R⁹ is CH₂F, R¹⁰ is CH₃ and the meaning of R⁴for each individual compound corresponds in each case to one line of Table A (compounds X-2.2-4.A-1 to X-2.2-4.A-35).

Table 2-5 Compounds of the formula X-2 in which R⁹ is CHF₂, R¹⁰ is Br and the meaning of R⁴ for each individual compound corresponds in each case to one line of Table A (compounds X-2.2-5.A-1 to X-2.2-5.A-35).

Table 2-6 Compounds of the formula X-2 in which R⁹ is CH₃, R¹⁰ is CH₂F and the meaning of R⁴for each individual compound corresponds in each case to one line of Table A (compounds X-2.2-6.A-1 to X-2.2-6.A-35).

Table 2-7 Compounds of the formula X-2 in which R⁹ is CHF₂, R¹⁰ is OCH₃ and the meaning of R⁴for each individual compound corresponds in each case to one line of Table A (compounds X-2.2-7.A-1 to X-2.2-7.A-35).

Table 3-1 Compounds of the formula X-3 in which R⁹ is CH₃, R¹⁰ is CH₃ and the meaning of R⁴for each individual compound corresponds in each case to one line of Table A (compounds X-3.3-1.A-1 to X-3.3-1.A-35).

Table 3-2 Compounds of the formula X-3 in which R⁹ is CHF₂, R¹⁰ is CH₃ and the meaning of R⁴for each individual compound corresponds in each case to one line of Table A (compounds X-3.3-2.A-1 to X-3.3-2.A-35).

Table 3-3 Compounds of the formula X-3 in which R⁹ is CH₃, R¹⁰ is C≡CH and the meaning of

R⁴for each individual compound corresponds in each case to one line of Table A (compounds X-3.3-3.A-1 to X-3.3-3.A-35).

Table 3-4 Compounds of the formula X-3 in which R⁹ is CH₂F, R¹⁰ is CH₃ and the meaning of R⁴for each individual compound corresponds in each case to one line of Table A (compounds X-3.3-4.A-1 to X-3.3-4.A-35).

Table 3-5 Compounds of the formula X-3 in which R⁹ is CHF₂, R¹⁰ is Br and the meaning of R⁴ for each individual compound corresponds in each case to one line of Table A (compounds X-3.3-5.A-1 to X-3.3-5.A-35).

Table 3-6 Compounds of the formula X-3 in which R⁹ is CH₃, R¹⁰ is CH₂F and the meaning of R⁴for each individual compound corresponds in each case to one line of Table A (compounds X-3.3-6.A-1 to X-3.3-6.A-35).

Table 3-7 Compounds of the formula X-3 in which R⁹ is CHF₂, R¹⁰ is OCH₃ and the meaning of R⁴for each individual compound corresponds in each case to one line of Table A (compounds X-3.3-7.A-1 to X-3.3-7.A-35).

Table 4-1 Compounds of the formula X-4 in which R⁹ is CH₃, R¹⁰ is CH₃ and the meaning of R⁴for each individual compound corresponds in each case to one line of Table A (compounds X-4.4-1.A-1 to X-4.4-1.A-35).

Table 4-2 Compounds of the formula X-4 in which R⁹ is CHF₂, R¹⁰ is CH₃ and the meaning of R⁴for each individual compound corresponds in each case to one line of Table A (compounds X-4.4-2.A-1 to X-4.4-2.A-35).

Table 4-3 Compounds of the formula X-4 in which R⁹ is CH₃, R¹⁰ is C≡CH and the meaning of R⁴for each individual compound corresponds in each case to one line of Table A (compounds X-4.4-3.A-1 to X-4.4-3.A-35).

Table 4-4 Compounds of the formula X-4 in which R⁹ is CH₂F, R¹⁰ is CH₃ and the meaning of R⁴for each individual compound corresponds in each case to one line of Table A (compounds X-4.4-4.A-1 to X-4.4-4.A-35).

Table 4-5 Compounds of the formula X-4 in which R⁹ is CHF₂, R¹⁰ is Br and the meaning of R⁴ for each individual compound corresponds in each case to one line of Table A (compounds X-4.4-5.A-1 to X-4.4-5.A-35).

Table 4-6 Compounds of the formula X-4 in which R⁹ is CH₃, R¹⁰ is CH₂F and the meaning of R⁴for each individual compound corresponds in each case to one line of Table A (compounds X-4.4-6.A-1 to X-4.4-6.A-35).

Table 4-7 Compounds of the formula X-4 in which R⁹ is CHF₂, R¹⁰ is OCH₃ and the meaning of R⁴for each individual compound corresponds in each case to one line of Table A (compounds X-4.4-7.A-1 to X-4.4-7.A-35).

Table 5-1 Compounds of the formula X-5 in which R⁹ is CH₃, R¹⁰ is CH₃ and the meaning of R⁴for each individual compound corresponds in each case to one line of Table A (compounds X-5.5-1.A-1 to X-5.5-1.A-35).

Table 5-2 Compounds of the formula X-5 in which R⁹ is CHF₂, R¹⁰ is CH₃ and the meaning of R⁴for each individual compound corresponds in each case to one line of Table A (compounds X-5.5-2.A-1 to X-5.5-2.A-35).

Table 5-3 Compounds of the formula X-5 in which R⁹ is CH₃, R¹⁰ is C≡CH and the meaning of R⁴for each individual compound corresponds in each case to one line of Table A (compounds X-5.5-3.A-1 to X-5.5-3.A-35).

Table 5-4 Compounds of the formula X-5 in which R⁹ is CH₂F, R¹⁰ is CH₃ and the meaning of R⁴for each individual compound corresponds in each case to one line of Table A (compounds X-5.5-4.A-1 to X-5.5-4.A-35).

Table 5-5 Compounds of the formula X-5 in which R⁹ is CHF₂, R¹⁰ is Br and the meaning of R⁴ for each individual compound corresponds in each case to one line of Table A (compounds X-5.5-5.A-1 to X-5.5-5.A-35).

Table 5-6 Compounds of the formula X-5 in which R⁹ is CH₃, R¹⁰ is CH₂F and the meaning of R⁴for each individual compound corresponds in each case to one line of Table A (compounds X-5.5-6.A-1 to X-5.5-6.A-35).

Table 5-7 Compounds of the formula X-5 in which R⁹ is CHF₂, R¹⁰ is OCH₃ and the meaning of R⁴for each individual compound corresponds in each case to one line of Table A (compounds X-5.5-7.A-1 to X-5.5-7.A-35).

Table 6-1 Compounds of the formula X-6 in which R⁹ is CH₃, R¹⁰ is CH₃ and the meaning of R⁴for each individual compound corresponds in each case to one line of Table A (compounds X-6.6-1.A-1 to X-6.6-1.A-35).

Table 6-2 Compounds of the formula X-6 in which R⁹ is CHF₂, R¹⁰ is CH₃ and the meaning of R⁴for each individual compound corresponds in each case to one line of Table A (compounds X-6.6-2.A-1 to X-6.6-2.A-35).

Table 6-3 Compounds of the formula X-6 in which R⁹ is CH₃, R¹⁰ is C≡CH and the meaning of R⁴for each individual compound corresponds in each case to one line of Table A (compounds X-6.6-3.A-1 to X-6.6-3.A-35).

Table 6-4 Compounds of the formula X-6 in which R⁹ is CH₂F, R¹⁰ is CH₃ and the meaning of R⁴for each individual compound corresponds in each case to one line of Table A (compounds X-6.6-4.A-1 to X-6.6-4.A-35).

Table 6-5 Compounds of the formula X-6 in which R⁹ is CHF₂, R¹⁰ is Br and the meaning of R⁴ for each individual compound corresponds in each case to one line of Table A (compounds X-6.6-5.A-1 to X-6.6-5.A-35).

Table 6-6 Compounds of the formula X-6 in which R⁹ is CH₃, R¹⁰ is CH₂F and the meaning of R⁴for each individual compound corresponds in each case to one line of Table A (compounds X-6.6-6.A-1 to X-6.6-6.A-35).

Table 6-7 Compounds of the formula X-6 in which R⁹ is CHF₂, R¹⁰ is OCH₃ and the meaning of R⁴for each individual compound corresponds in each case to one line of Table A (compounds X-6.6-7.A-1 to X-6.6-7.A-35).

Table 7-1 Compounds of the formula X-7 in which R⁹ is CH₃, R¹⁰ is CH₃ and the meaning of R⁴for each individual compound corresponds in each case to one line of Table A (compounds X-7.7-1.A-1 to X-7.7-1.A-35).

Table 7-2 Compounds of the formula X-7 in which R⁹ is CHF₂, R¹⁰ is CH₃ and the meaning of R⁴for each individual compound corresponds in each case to one line of Table A (compounds X-7.7-2.A-1 to X-7.7-2.A-35).

Table 7-3 Compounds of the formula X-7 in which R⁹ is CH₃, R¹⁰ is C≡CH and the meaning of R⁴for each individual compound corresponds in each case to one line of Table A (compounds X-7.7-3.A-1 to X-7.7-3.A-35).

Table 7-4 Compounds of the formula X-7 in which R⁹ is CH₂F, R¹⁰ is CH₃ and the meaning of R⁴for each individual compound corresponds in each case to one line of Table A (compounds X-7.7-4.A-1 to X-7.7-4.A-35).

Table 7-5 Compounds of the formula X-7 in which R⁹ is CHF₂, R¹⁰ is Br and the meaning of R⁴ for each individual compound corresponds in each case to one line of Table A (compounds X-7.7-5.A-1 to X-7.7-5.A-35).

Table 7-6 Compounds of the formula X-7 in which R⁹ is CH₃, R¹⁰ is CH₂F and the meaning of R⁴for each individual compound corresponds in each case to one line of Table A (compounds X-7.7-6.A-1 to X-7.7-6.A-35).

Table 7-7 Compounds of the formula X-7 in which R⁹ is CHF₂, R¹⁰ is OCH₃ and the meaning of R⁴for each individual compound corresponds in each case to one line of Table A (compounds X-7.7-7.A-1 to X-7.7-7.A-35).

Table 8-1 Compounds of the formula X-8 in which R⁹ is CH₃, R¹⁰ is CH₃ and the meaning of R⁴for each individual compound corresponds in each case to one line of Table A (compounds X-8.8-1.A-1 to X-8.8-1.A-35).

Table 8-2 Compounds of the formula X-8 in which R⁹ is CHF₂, R¹⁰ is CH₃ and the meaning of R⁴for each individual compound corresponds in each case to one line of Table A (compounds X-8.8-2.A-1 to X-8.8-2.A-35).

Table 8-3 Compounds of the formula X-8 in which R⁹ is CH₃, R¹⁰ is C≡CH and the meaning of R⁴for each individual compound corresponds in each case to one line of Table A (compounds X-8.8-3.A-1 to X-8.8-3.A-35).

Table 8-4 Compounds of the formula X-8 in which R⁹ is CH₂F, R¹⁰ is CH₃ and the meaning of R⁴for each individual compound corresponds in each case to one line of Table A (compounds X-8.8-4.A-1 to X-8.8-4.A-35).

Table 8-5 Compounds of the formula X-8 in which R⁹ is CHF₂, R¹⁰ is Br and the meaning of R⁴ for each individual compound corresponds in each case to one line of Table A (compounds X-8.8-5.A-1 to X-8.8-5.A-35).

Table 8-6 Compounds of the formula X-8 in which R⁹ is CH₃, R¹⁰ is CH₂F and the meaning of R⁴for each individual compound corresponds in each case to one line of Table A (compounds X-8.8-6.A-1 to X-8.8-6.A-35).

Table 8-7 Compounds of the formula X-8 in which R⁹ is CHF₂, R¹⁰ is OCH₃ and the meaning of R⁴for each individual compound corresponds in each case to one line of Table A (compounds X-8.8-7.A-1 to X-8.8-7.A-35).

Table 9-1 Compounds of the formula X-9 in which R⁹ is CH₃, R¹⁰ is CH₃ and the meaning of R⁴for each individual compound corresponds in each case to one line of Table A (compounds X-9.9-1.A-1 to X-9.9-1.A-35).

Table 9-2 Compounds of the formula X-9 in which R⁹ is CHF₂, R¹⁰ is CH₃ and the meaning of R⁴for each individual compound corresponds in each case to one line of Table A (compounds X-9.9-2.A-1 to X-9.9-2.A-35).

Table 9-3 Compounds of the formula X-9 in which R⁹ is CH₃, R¹⁰ is C≡CH and the meaning of R⁴for each individual compound corresponds in each case to one line of Table A (compounds X-9.9-3.A-1 to X-9.9-3.A-35).

Table 9-4 Compounds of the formula X-9 in which R⁹ is CH₂F, R¹⁰ is CH₃ and the meaning of R⁴for each individual compound corresponds in each case to one line of Table A (compounds X-9.9-4.A-1 to X-9.9-4.A-35).

Table 9-5 Compounds of the formula X-9 in which R⁹ is CHF₂, R¹⁰ is Br and the meaning of R⁴ for each individual compound corresponds in each case to one line of Table A (compounds X-9.9-5.A-1 to X-9.9-5.A-35).

Table 9-6 Compounds of the formula X-9 in which R⁹ is CH₃, R¹⁰ is CH₂F and the meaning of R⁴for each individual compound corresponds in each case to one line of Table A (compounds X-9.9-6.A-1 to X-9.9-6.A-35).

Table 9-7 Compounds of the formula X-9 in which R⁹ is CHF₂, R¹⁰ is OCH₃ and the meaning of R⁴for each individual compound corresponds in each case to one line of Table A (compounds X-9.9-7.A-1 to X-9.9-7.A-35).

Table 10-1 Compounds of the formula X-10 in which R⁹ is CH₃, R¹⁰ is CH₃ and the meaning of R⁴for each individual compound corresponds in each case to one line of Table A (compounds X-10.10-1.A-1 to X-10.10-1.A-35).

Table 10-2 Compounds of the formula X-10 in which R⁹ is CHF₂, R¹⁰ is CH₃ and the meaning of R⁴for each individual compound corresponds in each case to one line of Table A (compounds X-10.10-2.A-1 to X-10.10-2.A-35).

Table 10-3 Compounds of the formula X-10 in which R⁹ is CH₃, R¹⁰ is C≡CH and the meaning of R⁴for each individual compound corresponds in each case to one line of Table A (compounds X-10.10-3.A-1 to X-10.10-3.A-35).

Table 10-4 Compounds of the formula X-10 in which R⁹ is CH₂F, R¹⁰ is CH₃ and the meaning of R⁴for each individual compound corresponds in each case to one line of Table A (compounds X-10.10-4.A-1 to X-10.10-4.A-35).

Table 10-5 Compounds of the formula X-10 in which R⁹ is CHF₂, R¹⁰ is Br and the meaning of R⁴ for each individual compound corresponds in each case to one line of Table A (compounds X-10.10-5.A-1 to X-10.10-5.A-35).

Table 10-6 Compounds of the formula X-10 in which R⁹ is CH₃, R¹⁰ is CH₂F and the meaning of

R⁴for each individual compound corresponds in each case to one line of Table A (compounds X-10.10-6.A-1 to X-10.10-6.A-35).

Table 10-7 Compounds of the formula X-10 in which R⁹ is CHF₂, R¹⁰ is OCH₃ and the meaning of R⁴for each individual compound corresponds in each case to one line of Table A (compounds X-10.10-7.A-1 to X-10.10-7.A-35).

Table 11-1 Compounds of the formula X-11 in which R⁹ is CH₃, R¹⁰ is CH₃ and the meaning of R⁴for each individual compound corresponds in each case to one line of Table A (compounds X-11.11-1.A-1 to X-11.11-1.A-35).

Table 11-2 Compounds of the formula X-11 in which R⁹ is CHF₂, R¹⁰ is CH₃ and the meaning of R⁴for each individual compound corresponds in each case to one line of Table A (compounds X-11.11-2.A-1 to X-11.11-2.A-35).

Table 11-3 Compounds of the formula X-11 in which R⁹ is CH₃, R¹⁰ is C≡CH and the meaning of R⁴for each individual compound corresponds in each case to one line of Table A (compounds X-11.11-3.A-1 to X-11.11-3.A-35).

Table 11-4 Compounds of the formula X-11 in which R⁹ is CH₂F, R¹⁰ is CH₃ and the meaning of R⁴for each individual compound corresponds in each case to one line of Table A (compounds X-11.11-4.A-1 to X-11.11-4.A-35).

Table 11-5 Compounds of the formula X-11 in which R⁹ is CHF₂, R¹⁰ is Br and the meaning of R⁴ for each individual compound corresponds in each case to one line of Table A (compounds X-11.11-5.A-1 to X-11.11-5.A-35).

Table 11-6 Compounds of the formula X-11 in which R⁹ is CH₃, R¹⁰ is CH₂F and the meaning of R⁴for each individual compound corresponds in each case to one line of Table A (compounds X-11.11-6.A-1 to X-11.11-6.A-35).

Table 11-7 Compounds of the formula X-11 in which R⁹ is CHF₂, R¹⁰ is OCH₃ and the meaning of R⁴for each individual compound corresponds in each case to one line of Table A (compounds X-11.11-7.A-1 to X-11.11-7.A-35).

Table 12-1 Compounds of the formula X-12 in which R⁹ is CH₃, R¹⁰ is CH₃ and the meaning of R⁴for each individual compound corresponds in each case to one line of Table A (compounds X-12.12-1.A-1 to X-12.12-1.A-35).

Table 12-2 Compounds of the formula X-12 in which R⁹ is CHF₂, R¹⁰ is CH₃ and the meaning of R⁴for each individual compound corresponds in each case to one line of Table A (compounds X-12.12-2.A-1 to X-12.12-2.A-35).

Table 12-3 Compounds of the formula X-12 in which R⁹ is CH₃, R¹⁰ is C≡CH and the meaning of R⁴for each individual compound corresponds in each case to one line of Table A (compounds X-12.12-3.A-1 to X-12.12-3.A-35).

Table 12-4 Compounds of the formula X-12 in which R⁹ is CH₂F, R¹⁰ is CH₃ and the meaning of R⁴for each individual compound corresponds in each case to one line of Table A (compounds X-12.12-4.A-1 to X-12.12-4.A-35).

Table 12-5 Compounds of the formula X-12 in which R⁹ is CHF₂, R¹⁰ is Br and the meaning of R⁴ for each individual compound corresponds in each case to one line of Table A (compounds X-12.12-5.A-1 to X-12.12-5.A-35).

Table 12-6 Compounds of the formula X-12 in which R⁹ is CH₃, R¹⁰ is CH₂F and the meaning of R⁴for each individual compound corresponds in each case to one line of Table A (compounds X-12.12-6.A-1 to X-12.12-6.A-35).

Table 12-7 Compounds of the formula X-12 in which R⁹ is CHF₂, R¹⁰ is OCH₃ and the meaning of R⁴for each individual compound corresponds in each case to one line of Table A (compounds X-12.12-7.A-1 to X-12.12-7.A-35).

TABLE A

| No. | R⁴ |
|---|---|
| A-1 | CF₃ |
| A-2 | CHF₂ |
| A-3 | CH₂F |
| A-4 | CN |
| A-5 | CH₂CN |
| A-6 | CH₂OCH₃ |
| A-7 | CH₂NMe₂ |
| A-8 | 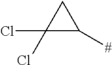 |
| | (C8) |
| A-9 | 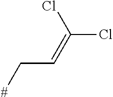 |
| A-10 | 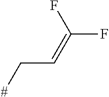 |
| A-11 | 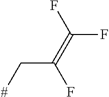 |
| A-12 | 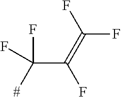 |
| A-13 | 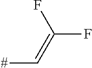 |
| A-14 | 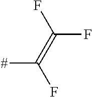 |
| A-15 | 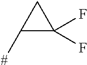 |
| A-16 | 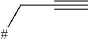 |
| A-17 | 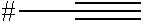 |
| A-18 | 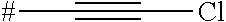 |
| A-19 | C₆H₅ |
| A-20 | 4-F—C₆H₄ |
| A-21 | CH₂—C₆H₅ |
| A-22 | CH₂—C₆H₄-4-F |
| A-23 | 2-py |
| A-24 | 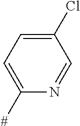 |
| A-25 | 3-py |
| A-26 | 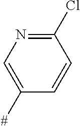 |
| A-27 | 4-py |
| A-28 | 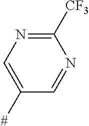 |
| A-29 | 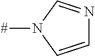 |
| A-30 | 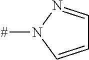 |

The compounds I and the compositions according to the invention, respectively, are suitable as fungicides. They are distinguished by an outstanding effectiveness against a broad spectrum of phytopathogenic fungi, including soil-borne fungi, which derive especially from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Some are systemically effective and they can be used in crop protection as foliar fungicides, fungicides for seed dressing and soil fungicides. Moreover, they are suitable for controlling harmful fungi, which inter alia occur in wood or roots of plants.

The compounds I and the compositions according to the invention are particularly important in the control of a multitude of phytopathogenic fungi on various cultivated plants, such as cereals, e.g. wheat, rye, barley, triticale, oats or rice; beet, e.g. sugar beet or fodder beet; fruits, such as pomes, stone fruits or soft fruits, e.g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; sweet leaf (also called Stevia); natural rubber plants or ornamental and forestry plants, such as flowers, shrubs, broad-leaved trees or evergreens, e.g. conifers; and on the plant propagation material, such as seeds, and the crop material of these plants.

Preferably, compounds I and compositions thereof, respectively are used for controlling a multitude of fungi on field crops, such as potatoes sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. These young plants may also be protected before transplantation by a total or partial treatment by immersion or pouring.

Preferably, treatment of plant propagation materials with compounds I and compositions thereof, respectively, is used for controlling a multitude of fungi on cereals, such as wheat, rye, barley and oats; rice, corn, cotton and soybeans.

The term “cultivated plants” is to be understood as including plants which have been modified by breeding, mutagenesis or genetic engineering including but not limiting to agricultural biotech products on the market or in development (cf. http://cera-gmc.org/, see GM crop database therein). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e.g. by glycosylation or polymer additions such as prenylated, acetylated or farnesylated moieties or PEG moieties.

Plants that have been modified by breeding, mutagenesis or genetic engineering, e.g. have been rendered tolerant to applications of specific classes of herbicides, such as auxin herbicides such as dicamba or 2,4-D; bleacher herbicides such as hydroxylphenylpyruvate dioxygenase (HPPD) inhibitors or phytoene desaturase (PDS) inhibitors; acetolactate synthase (ALS) inhibitors such as sulfonyl ureas or imidazolinones; enolpyruvylshikimate-3-phosphate synthase (EPSPS) inhibitors, such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i. e. bromoxynil or ioxynil) herbicides as a result of conventional methods of breeding or genetic engineering. Furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are e.g. described in Pest Managem. Sci. 61, 2005, 246; 61, 2005, 258; 61, 2005, 277; 61, 2005, 269; 61, 2005, 286; 64, 2008, 326; 64, 2008, 332; Weed Sci. 57, 2009, 108; Austral. J. Agricult. Res. 58, 2007, 708; Science 316, 2007, 1185; and references quoted therein. Several cultivated plants have been rendered tolerant to herbicides by conventional methods of breeding (mutagenesis), e.g. Clearfield® summer rape (Canola, BASF SE, Germany) being tolerant to imidazolinones, e.g. imazamox, or ^{ExpressSun}® sunflowers (DuPont, USA) being tolerant to sulfonyl ureas, e.g. tribenuron. Genetic engineering methods have been used to render cultivated plants such as soybean, cotton, corn, beets and rape, tolerant to herbicides such as glyphosate and glufosinate, some of which are commercially available under the trade names RoundupReady® (glyphosate-tolerant, Monsanto, U.S.A.), Cultivance® (imidazolinone tolerant, BASF SE, Germany) and LibertyLink® (glufosinate-tolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as 6-endotoxins, e.g. CryIA(b), CryIA(c), CryIF, CryIF(a2), CryIIA(b), CryIIIA, CryIIIB(b1) or Cry9c; vegetative insecticidal proteins (VIP), e.g. VIP1, VIP2, VIP3 or VIP3A; insecticidal proteins of bacteria colonizing nematodes, e.g. Photorhabdusspp. or Xenorhabdusspp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdyster-oid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilbene synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e.g. WO 02/015701). Further examples of such toxins or genetically modified plants capable of synthesizing such toxins are disclosed, e.g., in EP-A 374 753, WO 93/007278, WO 95/34656, EP-A 427 529, EP-A 451 878, WO 03/18810 and WO 03/52073. The methods for producing such genetically modified plants are generally known to the person skilled in the art and are described, e.g. in the publications mentioned above. These insecticidal proteins contained in the genetically modified plants impart to the plants producing these proteins tolerance to harmful pests from all taxonomic groups of arthropods, especially to beetles (Coeloptera), two-winged insects (Diptera), and moths (Lepidoptera) and to nematodes (Nematoda). Genetically modified plants capable to synthesize one or more insecticidal proteins are, e.g., described in the publications mentioned above, and some of which are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme phosphinothricin-N-acetyltransferase [PAT]); NuCOTN® 33B (cotton cultivars producing the CrylAc toxin), Bollgard® I (cotton cultivars producing the Cry1Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Bt11 (e.g. Agrisure® CB) and Bt176 from Syngenta Seeds SAS, France, (corn cultivars producing the Cry1Ab toxin and PAT enyzme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1F toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e.g. EP-A 392 225), plant disease resistance genes (e.g. potato cultivars, which express resistance genes acting against Phytophthora infestans derived from the Mexican wild potato Solanum bulbocastanum) or T4-lysozym (e.g. potato cultivars capable of synthesizing these proteins with increased resistance against bacteria such as Erwinia amylvora). The methods for producing such genetically modified plants are generally known to the person skilled in the art and are described, e.g. in the publications mentioned above.

Furthermore, plants are also covered that are by the use of recombinant DNA techniques ca-pable to synthesize one or more proteins to increase the productivity (e.g. bio mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of substances of content or new substances of content, specifically to improve human or animal nutrition, e.g. oil crops that produce health-promoting long-chain ome-ga-3 fatty acids or unsaturated omega-9 fatty acids (e.g. Nexera® rape, DOW Agro Sciences, Canada).

Furthermore, plants are also covered that contain by the use of recombinant DNA techniques a modified amount of substances of content or new substances of content, specifically to improve raw material production, e.g. potatoes that produce increased amounts of amylopectin (e.g. Amflora® potato, BASF SE, Germany).

The compounds I and compositions thereof, respectively, are particularly suitable for controlling the following plant diseases:

Albugo spp. (white rust) on ornamentals, vegetables (e.g. A. candida) and sunflowers (e.g. A. tragopogonis); Altemaria spp. (Alternaria leaf spot) on vegetables, rape (A. brassicola or brassicae), sugar beets (A. tenuis), fruits, rice, soybeans, potatoes (e.g. A. solani or A. alternata), tomatoes (e.g. A. solani or A. altemata) and wheat; Aphanomyces spp. on sugar beets and vegetables; Ascochyta spp. on cereals and vegetables, e.g. A. tritici (anthracnose) on wheat and A. hordei on barley; Bipolaris and Drechslera spp. (teleomorph: Cochliobolus spp.), e.g. Southern leaf blight (D. maydis) or Northern leaf blight (B. zeicola) on corn, e.g. spot blotch (B. sorokiniana) on cereals and e.g. B. oryzae on rice and turfs; Blumeria (formerly Erysiphe) graminis (powdery mildew) on cereals (e.g. on wheat or barley); Botrytis cinerea (teleomorph: Botryotinia fuckeliana: grey mold) on fruits and berries (e.g. strawberries), vegetables (e.g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e.g. C. ulmi (Dutch elm disease) on elms; Cercospora spp. (Cercospora leaf spots) on corn (e.g. Gray leaf spot: C. zeae-maydis), rice, sugar beets (e.g. C. beticola), sugar cane, vegetables, coffee, soybeans (e.g. C. sojina or C. kikuchii) and rice; Cladosporium spp. on tomatoes (e.g. C. fulvum: leaf mold) and cereals, e.g. C. herbarum (black ear) on wheat; Claviceps purpurea (ergot) on cereals; Cochliobolus (anannorph: Helminthosporium of Bipolaris) spp. (leaf spots) on corn (C. carbonum), cereals (e.g. C. sativus, anamorph: B. sorokiniana) and rice (e.g. C. miyabeanus, anamorph: H. oryzae); Colletotrichum (teleomorph: Glomerella) spp. (anthracnose) on cotton (e.g. C. gossypii), corn (e.g. C. graminicola: Anthracnose stalk rot), soft fruits, potatoes (e.g. C. coccodes, black dot), beans (e.g. C. lindemuthianum) and soybeans (e.g. C. truncatum or C. gloeosporioides); Corticium spp., e.g. C. sasakil (sheath blight) on rice; Corynespora cassiicola (leaf spots) on soybeans and ornamentals; Cycloconium spp., e.g. C. oleaginum on olive trees; Cylindrocarpon spp. (e.g. fruit tree canker or young vine decline, teleomorph: Nectria or Neonectria spp.) on fruit trees, vines (e.g. C. liriodendri, teleomorph: Neonectria liriodendri, Black Foot Disease) and ornamentals; Dematophora (teleomorph: Rosellinia) necatrix (root and stem rot) on soybeans; Diaporthe spp., e.g. D. phaseolorum (damping off) on soybeans; Drechslera (syn. Helminthosporium, teleomorph: Pyrenophora) spp. on corn, cereals, such as barley (e.g. D. teres, net blotch) and wheat (e.g. D. tritici-repentis: tan spot), rice and turf; Esca (dieback, apoplexy) on vines, caused by Formitipona (syn. Phellinus) punctata, F. mediterranea, Phaeomoniella chlamydospora (earlier Phaeoacremonium chlamydosporum), Phaeoacremonium aleophilum and/or Botryosphaeria obtusa; Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e.g. E. pisi), such as cucurbits (e.g. E. cichoracearum), cabbages, rape (e.g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e.g. E. turcicum); Fusarium (teleomorph: Gibberella) spp. (wilt, root or stem rot) on various plants, such as F. graminearum or F. culmorum (root rot, scab or head blight) on cereals (e.g. wheat or barley), F. oxysporum on tomatoes, F. solani (f. sp. glycines now syn. F. virguliforme) and F. tucumaniae and F. brasiliense each causing sudden death syndrome on soybeans, and F. verticillioides on corn; Gaeumannomyces graminis (take-all) on cereals (e.g. wheat or barley) and corn; Gibberella spp. on cereals (e.g. G. zeae) and rice (e.g. G. fujikuror: Bakanae disease); Glomerella cingulata on vines, pome fruits and other plants and G. gossypii on cotton; Grainstaining complex on rice; Guignardia bidwellii (black rot) on vines; Gymnosporangium spp. on rosaceous plants and junipers, e.g. G. sabinae (rust) on pears; Helminthosporium spp. (syn. Drechslera, teleomorph: Cochliobolus) on corn, cereals and rice; Hemileia spp., e.g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e.g. wheat or barley); Microsphaera diffusa (powdery mildew) on soybeans; Monilinia spp., e.g. M. laxa, M. fructicola and M. fructigena (bloom and twig blight, brown rot) on stone fruits and other rosaceous plants; Mycosphaerella spp. on cereals, bananas, soft fruits and ground nuts, such as e.g. M. graminicola (anamorph: Septoria tritici, Septoria blotch) on wheat or M. fijiensis (black Sigatoka disease) on bananas; Peronospora spp. (downy mildew) on cabbage (e.g. P. brassicae), rape (e.g. P. parasitica), onions (e.g. P. destructor), tobacco (P. tabacina) and soybeans (e.g. P. manshurica); Phakopsora pachyrhizi and P. meibomiae (soybean rust) on soybeans; Phialophora spp. e.g. on vines (e.g. P. tracheiphila and P. tetraspora) and soybeans (e.g. P. gregata: stem rot); Phoma lingam (root and stem rot) on rape and cabbage and P. betae (root rot, leaf spot and damping-off) on sugar beets; Phomopsis spp. on sunflowers, vines (e.g. P. viticola: can and leaf spot) and soybeans (e.g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e.g. P. capsici), soybeans (e.g. P. megasperma, syn. P. sojae), potatoes and tomatoes (e.g. P. infestans late blight) and broad-leaved trees (e.g. P. ramorum: sudden oak death); Plasmodiophora brassicae (club root) on cabbage, rape, radish and other plants; Plasmopara spp., e.g. P. viticola (grapevine downy mildew) on vines and P. halstedii on sunflowers; Podosphaera spp. (powdery mildew) on rosaceous plants, hop, pome and soft fruits, e.g. P. leucotricha on apples; Polymyxa spp., e.g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e.g. wheat or barley; Pseudoperonospora (downy mildew) on various plants, e.g. P. cubensis on cucurbits or P. humili on hop; Pseudopezicula tracheiphlla (red fire disease or rotbrenner', anamorph: Phialophora) on vines; Puccinia spp. (rusts) on various plants, e.g. P. triticina (brown or leaf rust), P. striiformis (stripe or yellow rust), P. hordei (dwarf rust), P. graminis (stem or black rust) or P. recondita (brown or leaf rust) on cereals, such as e.g. wheat, barley or rye, P. kuehnii (orange rust) on sugar cane and P. asparagi on asparagus; Pyrenophora (anamorph: Drechslera) tritici-repentis (tan spot) on wheat or P. teres (net blotch) on barley; Pyricularia spp., e.g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e.g. P. ultimum or P. aphanidermatu); Ramularia spp., e.g. R. collo-cygni (Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e.g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotinia spp. (stem rot or white mold) on vegetables and field crops, such as rape, sunflowers (e.g. S. sclerotiorum) and soybeans (e.g. S. rolfsii or S. sclerotiorum); Septoria spp. on various plants, e.g. S. glycines (brown spot) on soybeans, S. tritici (Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setospaeria spp. (leaf blight) on corn (e.g. S. turcicum, syn. Helminthosporium turcicum) and turf; Sphacelotheca spp. (smut) on corn, (e.g. S. reliana: head smut), sorghum and sugar cane; Sphaerotheca fuliginea (powdery mildew) on cucurbits; Spongospora subterranea (powdery scab) on potatoes and thereby transmitted viral diseases; Stagonospora spp. on cereals, e.g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e.g. T. deformans (leaf curl disease) on peaches and T. pruni (plum pocket) on plums; Thielaviopsis spp. (black root rot) on tobacco, pome fruits, vegetables, soybeans and cotton, e.g. T. basicola (syn. Chalara elegans); Tilletia spp. (common bunt or stinking smut) on cereals, such as e.g. T. tritici (syn. T. caries, wheat bunt) and T. controversa (dwarf bunt) on wheat; Typhula incarnata (grey snow mold) on barley or wheat; Urocystis spp., e.g. U. occulta (stem smut) on rye; Uromyces spp. (rust) on vegetables, such as beans (e.g. U. appendiculatus, syn. U. phaseoli) and sugar beets (e.g. U. betae); Ustilago spp. (loose smut) on cereals (e.g. U. nuda and U. avaenae), corn (e.g. U. maydis: corn smut) and sugar cane; Venturia spp. (scab) on apples (e.g. V. inaequalis) and pears; and Verticillium spp. (wilt) on various plants, such as fruits and ornamentals, vines, soft fruits, vegetables and field crops, e.g. V. dahliae on strawberries, rape, potatoes and tomatoes.

The compounds I and compositions thereof, respectively, are also suitable for controlling harmful fungi in the protection of stored products or harvest and in the protection of materials. The term “protection of materials” is to be understood to denote the protection of technical and non-living materials, such as adhesives, glues, wood, paper and paperboard, textiles, leather, paint dispersions, plastics, cooling lubricants, fiber or fabrics, against the infestation and destruction by harmful microorganisms, such as fungi and bacteria. As to the protection of wood and other materials, the particular attention is paid to the following harmful fungi: Ascomycetes such as Ophiostoma spp., Ceratocystis spp., Aureobasidium pullulans, Scierophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichoderma spp., Alternaria spp., Paecilomyces spp. and Zygomycetes such as Mucor spp., and in addition in the protection of stored products and harvest the following yeast fungi are worthy of note: Candida spp. and Saccharomyces cerevisae.

The method of treatment according to the invention can also be used in the field of protecting stored products or harvest against attack of fungi and microorganisms. According to the present invention, the term “stored products” is understood to denote natural substances of plant or animal origin and their processed forms, which have been taken from the natural life cycle and for which long-term protection is desired. Stored products of crop plant origin, such as plants or parts thereof, for example stalks, leafs, tubers, seeds, fruits or grains, can be protected in the freshly harvested state or in processed form, such as pre-dried, moistened, comminuted, ground, pressed or roasted, which process is also known as post-harvest treatment. Also falling under the definition of stored products is timber, whether in the form of crude timber, such as construction timber, electricity pylons and barriers, or in the form of finished articles, such as furniture or objects made from wood. Stored products of animal origin are hides, leather, furs, hairs and the like. The combinations according the present invention can prevent disadvantageous effects such as decay, discoloration or mold. Preferably “stored products” is understood to denote natural substances of plant origin and their processed forms, more preferably fruits and their processed forms, such as pomes, stone fruits, soft fruits and citrus fruits and their processed forms.

The compounds I and compositions thereof, respectively, may be used for improving the health of a plant. The invention also relates to a method for improving plant health by treating a plant, its propagation material and/or the locus where the plant is growing or is to grow with an effective amount of compounds I and compositions thereof, respectively.

The term “plant health” is to be understood to denote a condition of the plant and/or its products which is determined by several indicators alone or in combination with each other such as yield (e.g. increased biomass and/or increased content of valuable ingredients), plant vigor (e.g. improved plant growth and/or greener leaves (“greening effect”)), quality (e.g. improved content or composition of certain ingredients) and tolerance to abiotic and/or biotic stress. The above identified indicators for the health condition of a plant may be interdependent or may result from each other.

The compounds of formula I can be present in different crystal modifications whose biological activity may differ. They are likewise subject matter of the present invention.

The compounds I are employed as such or in form of compositions by treating the fungi or the plants, plant propagation materials, such as seeds, soil, surfaces, materials or rooms to be protected from fungal attack with a fungicidally effective amount of the active substances. The application can be carried out both before and after the infection of the plants, plant propagation materials, such as seeds, soil, surfaces, materials or rooms by the fungi.

Plant propagation materials may be treated with compounds I as such or a composition comprising at least one compound I prophylactically either at or before planting or transplanting.

The invention also relates to agrochemical compositions comprising an auxiliary and at least one compound I according to the invention.

An agrochemical composition comprises a fungicidally effective amount of a compound I. The term “effective amount” denotes an amount of the composition or of the compounds I, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific compound I used.

The compounds I, their N-oxides and salts can be converted into customary types of agrochemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Monograph No. 2, 6^{th} Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e.g. toluene, paraffin, tetrahydronaphthalene, alkylated naphthalenes; alcohols, e.g. ethanol, propanol, butanol, benzyl alcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methyl pyrrolidone, fatty acid dimethyl amides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol.1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylaryl sulfonates, diphenyl sulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyl naphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinyl pyrrolidone, vinyl alcohols, or vinyl acetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinyl amines or polyethylene amines.

Suitable adjuvants are compounds, which have a negligible or even no pesticidal activity themselves, and which improve the biological performance of the compound I on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e.g. xanthan gum, carboxymethyl cellulose), inorganic clays (organically modified or unmodified), polycarboxylates, and silicates. Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinyl pyrrolidones, polyvinyl acetates, polyvinyl alcohols, polyacrylates, biological or synthetic waxes, and cellulose ethers.

Examples for composition types and their preparation are:

i) Water-Soluble Concentrates (SL, LS)

10-60 wt % of a compound I and 5-15 wt % wetting agent (e.g. alcohol alkoxylates) are dissolved in water and/or in a water-soluble solvent (e.g. alcohols) ad 100 wt %. The active substance dissolves upon dilution with water.

ii) Dispersible Concentrates (DC)

5-25 wt % of a compound I and 1-10 wt % dispersant (e.g. polyvinyl pyrrolidone) are dissolved in organic solvent (e.g. cyclohexanone) ad 100 wt %. Dilution with water gives a dispersion.

iii) Emulsifiable Concentrates (EC)

15-70 wt % of a compound I and 5-10 wt % emulsifiers (e.g. calcium dodecylbenzenesulfonate and castor oil ethoxylate) are dissolved in water-insoluble organic solvent (e.g. aromatic hydro-carbon) ad 100 wt %. Dilution with water gives an emulsion.

vi) Emulsions (EW, EO, ES)

5-40 wt % of a compound I and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into water ad 100 wt % by means of an emulsifying machine and made into a homogeneous emulsion. Dilution with water gives an emulsion.

v) Suspensions (SC, OD, FS)

In an agitated ball mill, 20-60 wt % of a compound I are comminuted with addition of 2-10 wt % dispersants and wetting agents (e.g. sodium lignosulfonate and alcohol ethoxylate), 0.1-2 wt % thickener (e.g. xanthan gum) and water ad 100 wt % to give a fine active substance suspension. Dilution with water gives a stable suspension of the active substance. For FS type composition up to 40 wt % binder (e.g. polyvinyl alcohol) is added.

vi) Water-Dispersible Granules and Water-Soluble Granules (WG, SG)

50-80 wt % of a compound I are ground finely with addition of dispersants and wetting agents (e.g. sodium lignosulfonate and alcohol ethoxylate) ad 100 wt % and prepared as water-dispersible or water-soluble granules by means of technical appliances (e.g. extrusion, spray tower, fluidized bed). Dilution with water gives a stable dispersion or solution of the active substance.

vii) Water-Dispersible Powders and Water-Soluble Powders (WP, SP, WS)

50-80 wt % of a compound I are ground in a rotor-stator mill with addition of 1-5 wt % dispersants (e.g. sodium lignosulfonate), 1-3 wt % wetting agents (e.g. alcohol ethoxylate) and solid carrier (e.g. silica gel) ad 100 wt %. Dilution with water gives a stable dispersion or solution of the active substance.

viii) Gel (GW, GF)

In an agitated ball mill, 5-25 wt % of a compound I are comminuted with addition of 3-10 wt % dispersants (e.g. sodium lignosulfonate), 1-5 wt % thickener (e.g. carboxymethyl cellulose) and water ad 100 wt % to give a fine suspension of the active substance. Dilution with water gives a stable suspension of the active substance.

ix) Microemulsion (ME)

5-20 wt % of a compound I are added to 5-30 wt % organic solvent blend (e.g. fatty acid dimethyl amide and cyclohexanone), 10-25 wt % surfactant blend (e.g. alcohol ethoxylate and arylphenol ethoxylate), and water ad 100%. This mixture is stirred for 1 h to produce spontaneously a thermodynamically stable microemulsion.

x) Microcapsules (CS)

An oil phase comprising 5-50 wt % of a compound I, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization results in the formation of poly(meth)acrylate microcapsules. Alternatively, an oil phase comprising 5-50 wt % of a compound I according to the invention, 0-40 wt % water insoluble organic solvent (e.g. aromatic hydrocarbon), and an isocyanate monomer (e.g. diphenylmethene-4,4′-diisocyanatae) are dispersed into an aqueous solution of a protective colloid (e.g. polyvinyl alcohol). The addition of a polyamine (e.g. hexamethylenediamine) results in the formation of polyurea microcapsules. The monomers amount to 1-10 wt %. The wt % relate to the total CS composition.

xi) Dustable Powders (DP, DS)

1-10 wt % of a compound I are ground finely and mixed intimately with solid carrier (e.g. finely divided kaolin) ad 100 wt %.

xii) Granules (GR, FG)

0.5-30 wt % of a compound I is ground finely and associated with solid carrier (e.g. silicate) ad 100 wt %. Granulation is achieved by extrusion, spray-drying or fluidized bed.

xiii) Ultra-Low Volume Liquids (UL)

1-50 wt % of a compound I are dissolved in organic solvent (e.g. aromatic hydrocarbon) ad 100 wt %.

The compositions types i) to xiii) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably be-tween 0.1 and 90%, more preferably between 1 and 70%, and in particular between 10 and 60%, by weight of active substance. The active substances are employed in a purity of from 90% to 100%, preferably from 95% to 100% (according to NMR spectrum).

For the purposes of treatment of plant propagation materials, particularly seeds, solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC), and gels (GF) are usually employed. The compositions in question give, after two-to-tenfold dilution, active substance concentrations of from 0.01 to 60% by weight, preferably from 0.1 to 40%, in the ready-to-use preparations. Application can be carried out before or during sowing. Methods for applying compound I and compositions thereof, respectively, onto plant propagation material, especially seeds, include dressing, coating, pelleting, dusting, and soaking as well as in-furrow application methods. Preferably, compound I or the compositions thereof, respectively, are applied on to the plant propagation material by a method such that germination is not induced, e.g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha. In treatment of plant propagation materials such as seeds, e.g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propegation material (preferably seeds) are generally required.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e. g. herbicides, insecticides, fungicides, growth regulators, safeners, biopesticides) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

A pesticide is generally a chemical or biological agent (such as pestidal active ingredient, compound, composition, virus, bacterium, antimicrobial or disinfectant) that through its effect deters, incapacitates, kills or otherwise discourages pests. Target pests can include insects, plant pathogens, weeds, mollusks, birds, mammals, fish, nematodes (roundworms), and microbes that destroy property, cause nuisance, spread disease or are vectors for disease. The term “pesticide” includes also plant growth regulators that alter the expected growth, flowering, or reproduction rate of plants; defoliants that cause leaves or other foliage to drop from a plant, usually to facilitate harvest; desiccants that promote drying of living tissues, such as unwanted plant tops; plant activators that activate plant physiology for defense of against certain pests; safeners that reduce unwanted herbicidal action of pesticides on crop plants; and plant growth promoters that affect plant physiology e.g. to increase plant growth, biomass, yield or any other quality parameter of the harvestable goods of a crop plant.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

In still another embodiment of formula I, individual components of the composition according to the invention such as parts of a kit or parts of a binary or ternary mixture may be mixed by the user himself in a spray tank or any other kind of vessel used for applications (e.g. seed treater drums, seed pelleting machinery, knapsack sprayer) and further auxiliaries may be added, if appropriate.

Consequently, one embodiment of the invention is a kit for preparing a usable pesticidal composition, the kit comprising a) a composition comprising component 1) as defined herein and at least one auxiliary; and b) a composition comprising component 2) as defined herein and at least one auxiliary; and optionally c) a composition comprising at least one auxiliary and optionally a further active component 3) as defined herein.

Mixing the compounds I or the compositions comprising them in the use form as fungicides with other fungicides results in many cases in an expansion of the fungicidal spectrum of activity being obtained or in a prevention of fungicide resistance development. Furthermore, in many cases, synergistic effects are obtained.

The following list of pesticides II (e.g. pesticidally-active substances and biopesticides), in conjunction with which the compounds I can be used, is intended to illustrate the possible combinations but does not limit them:

A) Respiration Inhibitors

Inhibitors of complex III at Qₒ site (e.g. strobilurins): azoxystrobin (A.1.1), coumethoxy-strobin (A.1.2), coumoxystrobin (A.1.3), dimoxystrobin (A.1.4), enestroburin (A.1.5), fenamin-strobin (A.1.6), fenoxystrobin/flufenoxystrobin (A.1.7), fluoxastrobin (A.1.8), kresoxim-methyl (A.1.9), mandestrobin (A.1.10), metominostrobin (A.1.11), orysastrobin (A.1.12), picoxystrobin (A.1.13), pyraclostrobin (A.1.14), pyrametostrobin (A.1.15), pyraoxystrobin (A.1.16), trifloxystrobin (A.1.17), 2-(2-(3-(2,6-dichlorophenyl)-1-methyl-allylidenea-minooxy-methyl)-phenyl)-2-methoxyi-mino-N-methyl-acetamide (A.1.18), pyribencarb (A.1.19), triclopyricarb/chlorodincarb (A.1.20), famoxadone (A.1.21), fenamidone (A.1.21), methyl-N-[2-[(1,4-dimethyl-5-phenyl-pyrazol-3-yl)oxyl-methyl]phenyl]-N-methoxy-carbamate (A.1.22), 1-[3-chloro-2-[[1-(4-chlorophenyl)-1H-pyrazol-3-yl]oxymethyl]phenyl]-4-methyl-tetrazol-5-one (A.1.23), 1-[3-bromo-2[[1-(4-chlorophenyl)pyrazol-3-yl]oxymethyl]phenyl]-4-methyl-tetrazol-5-one (A.1.24), 1-[2-[[1-(4-chlorophenyl)pyrazol-3-yl]oxymethyl]-3-methyl-phenyl]-4-methyl-tetrazol-5-one (A.1.25), 1-[2-[[1-(4-chlorophenyl)pyrazol-3-yl]oxymethyl]-3-fluoro-phenyl]-4-methyl-tetrazol-5-one (A.1.26), 1-[2-[[1-(2,4-dichlorophenyl)pyrazol-3-yl]oxymethyl]-3-fluoro-phenyl]-4-methyl-tetrazol-5-one (A.1.27),

1-[2-[[4-(4-chlorophenyl)thiazol-2-yl]oxymethyl]-3-methyl-phenyl]-4-methyl-tetrazol-5-one (A.1.28), 1-[3-chloro-2-[[4-(p-tolyl)thiazol-2-yl]oxymethyl]phenyl]-4-methyl-tetrazol-5-one (A.1.29), 1-[3-cyclopropyl-2-[[2-methyl-4-(1-methylpyrazol-3-yl)phenoxy]methyl]phenyl]-4-methyl-tetrazol-5-one (A.1.30), 1-1-[3-(difluoromethoxy)-2-[[2-methyl-4-(1-methylpyrazol-3-yl)phenoxy]methyl]phenyl]-4-methyl-tetrazol-5-one (A.1.31), 1-methyl-4-[3-methyl-2-[[2-methyl-4-(1-methylpyrazol-3yl)phenoxy]methyl]phenyl]petrazol-5-one (A.1.32), 1-methyl-4-[3-methyl-2-[[1-[3-(trifluoromethyl)phenyl]-ethylideneamino]oxymethyl]phenyl]tetrazol-5-one (A.1.33), (Z2E)-5-[1-(2,4-dichlorophenyl)pyrazol-3-yl]-oxy-2-methoxyimino-N,3-dimethyl-pent-3-enamide (A.1.34), (Z,2E)-5-[1-(4-chlorophenyl)pyrazol-3-yl]oxy-2-methoxyimino-N,3-dimethyl-pent-3-enamide (A.1.35), (Z,2E)-5-[1-(4-chloro-2-fluoro-phenyl)pyrazol-3-yl]oxy-2-methoxyimino-N,3-dimethyl-pent-3-enamide (A.1.36),

inhibitors of complex III at Qᵢ site: cyazofamid (A.2.1), amisulbrom (A.2.2), [(3S,6S,7R,8R)-8-benzyl-3-[(3-acetoxy-4-methoxy-pyridine-2-carbonyl)annino]-6-methyl-4,9-dioxo-1,5-dioxonan-7-yl] 2-methylpropanoate (A.2.3), [(3S,6S,7R,8R)-8-benzyl-3-[[3-(acetoxy-methoxy)-4-methoxy-pyridine-2-carbonyl]amino]-6-methyl-4,9-dioxo-1,5-dioxonan-7-yl] 2-methylpropanoate (A.2.4), [(3S,6S,7R,8R)-8-benzyl-3-[(3-isobutoxycarbonyloxy-4-methoxy-pyridine-2-carbonyl)amino]-6-methyl-4,9-dioxo-1,5-dioxonan-7-yl] 2-methylpropanoate (A.2.5), [(3S,6S,7R,8R)-8-benzyl-3-[[3-(1,3-benzodioxol-5-ylmethoxy)-4-methoxy-pyridine-2-carbonyl]annino]-6-methyl-4,9-dioxo-1,5-dioxonan-7-yl] 2-methylpropanoate (A.2.6); (3S,6S,7R,8R)-3-[[(3-hydroxy-4-methoxy-2-pyridinyl)carbonyl]amino]-6-methyl-4,9-dioxo-8-(phenylmethyl)-1,5-dioxonan-7-yl 2-methylpropanoate (A.2.7), (3S,6S,7R,8R)-8-benzyl-3-[3-[(isobutyryloxy)methoxy]-4-methoxypicolinamido]-6-methyl-4,9-dioxo-1,5-dioxonan-7-yl isobutyrate (A.2.8);

inhibitors of complex II (e.g. carboxamides): benodanil (A.3.1), benzovindiflupyr (A.3.2), bixafen (A.3.3), boscalid (A.3.4), carboxin (A.3.5), fenfuram (A.3.6), fluopyram (A.3.7), flutolanil (A.3.8), fluxapyroxad (A.3.9), furametpyr (A.3.10), isofetamid (A.3.11), isopyrazam (A.3.12), mepronil (A.3.13), oxycarboxin (A.3.14), penflufen (A.3.14), penthiopyrad (A.3.15), sedaxane (A.3.16), tecloftalam (A.3.17), thifluzamide (A.3.18), N-(4′-trifluoro-methylthiobiphenyl-2-yl)-3-difluoromethyl-1-methyl-1H-pyrazole-4-carboxamide (A.3.19), N-(2-(1,3,3-trimethyl-butyl)-phenyl)-1,3-dimethyl-5-fluoro-1H-pyrazole-4-carboxamide (A.3.20), 3-(difluoromethyl)-1-methyl-N-(1,1,3-trimethylindan-4-yl)pyrazole-4-carboxamide (A.3.21), 3-(trifluoro-methyl)-1-methyl-N-(1,1,3-trimethylindan-4-yl)pyrazole-4-carboxamide (A.3.22), 1,3-dimethyl-N-(1,1,3-trimethylindan-4-yl)pyrazole-4-carboxamide (A.3.23), 3-(trifluoromethyl)-1,5-dimethyl-N-(1,1,3-trimethylindan-4-yl)pyrazole-4-carboxamide (A.3.24), 1,3,5-trimethyl-N-(1,1,3-trimethylindan-4-yl)pyrazole-4-carboxamide (A.3.25), N-(7-fluoro-1,1,3-trimethyl-indan-4-yl)-1,3-dimethyl-pyrazole-4-carboxamide (A.3.26), N-[2-(2,4-dichlorophenyl)-2-methoxy-1-methyl-ethyl]-3-(difluoromethyl)-1-methyl-pyrazole-4-carboxamide (A.3.27);

other respiration inhibitors (e.g. complex I, uncouplers): diflumetorim (A.4.1), (5,8-difluoro-quinazolin-4-yl)-{2-[2-fluoro-4-(4-trifluoro-methylpyridin-2-yloxy)-phenyl]-ethyl}-amine (A.4.2); nitrophenyl derivates: binapacryl (A.4.3), dinobuton (A.4.4), dinocap (A.4.5), fluazinam (A.4.6); ferimzone (A.4.7); organometal compounds: fentin salts, such as fentin-acetate (A.4.8), fentin chloride (A.4.9) or fentin hydroxide (A.4.10); ametoctradin (A.4.11); and silthiofam (A.4.12);

B) Sterol Biosynthesis Inhibitors (SBI Fungicides)

C14 demethylase inhibitors (DMI fungicides): triazoles: azaconazole (B.1.1), bitertanol (B.1.2), bromuconazole (B.1.3), cyproconazole (B.1.4), difenoconazole (B.1.5), diniconazole (B.1.6), diniconazole-M (B.1.7), epoxiconazole (B.1.8), fenbuconazole (B.1.9), fluquinconazole (B.1.10), flusilazole (B.1.11), flutriafol (B.1.12), hexaconazole (B.1.13), imibenconazole (B.1.14), ipconazole (B.1.15), metconazole (B.1.17), myclobutanil (B.1.18), oxpoconazole (B.1.19), paclobutrazole (B.1.20), penconazole (B.1.21), propiconazole (B.1.22), prothioconazole (B.1.23), simeconazole (B.1.24), tebuconazole (B.1.25), tetraconazole (B.1.26), triadimefon (B.1.27), triadimenol (B.1.28), triticonazole (B.1.29), uniconazole (B.1.30), 1-[rel-(2S,3R)-3-(2-chlorophenyl)-2-(2,4-difluorophenyl)-oxiranylmethyl]-5-thiocyanato-1H-[1,2,4]triazolo (B.1.31), 2-[rel-k(2S;3A)-3-(2-chlorophenyl)-2-(2,4-difluorophenyl)-oxiranylmethyl]-2H-[1 ,2,4]triazole-3-thiol (B.1.32), 2-[2-chloro-4-(4-chlorophenoxy)phenyl]-1-(1,2,4-triazol-1-yl)pentan-2-ol (B.1.33), 1-[4-(4-chlorophenoxy)-2-(trifluoromethyl)phenyl]-1-cyclopropyl-2-(1,2,4-triazol-1-yl)ethanol (B.1.34), 2-[4-(4-chlorophenoxy)-2-(trifluoromethyl)phenyl]-1-(1,2,4-triazol-1-yl)butan-2-ol (B.1.35), 2-[2-chloro-4-(4-chlorophenoxy)phenyl]-1-(1,2,4-triazol-1-yl)butan-2-ol (B.1.36), 2-[4-(4-chloro-phenoxy)-2-(trifluoromethyl)phenyl]-3-methyl-1-(1,2,4-triazol-1-yl)butan-2-ol (B.1.37), 2-[4-(4-chlorophenoxy)-2-(trifluoromethyl)phenyl]-1-(1,2,4-triazol-1-yl)propan-2-ol (B.1.38), 2-[2-chloro-4-(4-chlorophenoxy)phenyl]-3-methyl-1-(1,2,4-triazol-1-yl)butan-2-ol (B.1.39), 2-[4-(4-chlorophenoxy)-2-(trifluoromethyl)phenyl]-1-(1,2,4-triazol-1-yl)pentan-2-ol (B.1.40), 2-[4-(4-fluorophenoxy)-2-(trifluoromethyl)phenyl]-1-(1,2,4-triazol-1-yl)propan-2-ol (B.1.41), 2-[2-chloro-4-(4-chlorophenoxy)phenyl]-1-(1,2,4-triazol-1-yl)pent-3-yn-2-ol (B.1.51); imidazoles: imazalil (B.1.42), pefurazoate (B.1.43), prochloraz (B.1.44), triflumizol (B.1.45); pyrimidines, pyridines and piperazines: fenarimol (B.1.46), nuarimol (B.1.47), pyrifenox (B.1.48), triforine (B.1.49), [3-(4-chloro-2-fluoro-phenyl)-5-(2,4-difluorophenyl)isoxazol-4-yl]-(3-pyridyl)-methanol (B.1.50);

Delta 14-reductase inhibitors: aldimorph (B.2.1), dodemorph (B.2.2), dodemorph-acetate (B.2.3), fenpropimorph (B.2.4), tridemorph (B.2.5), fenpropidin (B.2.6), piperalin (B.2.7), spirox-amine (B.2.8);

Inhibitors of 3-keto reductase: fenhexamid (B.3.1);

C) Nucleic Acid Synthesis Inhibitors

phenylamides or acyl amino acid fungicides: benalaxyl (C.1.1), benalaxyl-M (C.1.2), kiralaxyl (C.1.3), metalaxyl (C.1.4), metalaxyl-M (mefenoxam, C.1.5), ofurace (C.1.6), oxadixyl (C.1.7);

others: hymexazole (C.2.1), octhilinone (C.2.2), oxolinic acid (C.2.3), bupirimate (C.2.4), 5-fluorocytosine (C.2.5), 5-fluoro-2-(p-tolylmethoxy)pyrimidin-4-amine (C.2.6), 5-fluoro-2-(4-fluorophenylmethoxy)pyrimidin-4-amine (C.2.7);

D) Inhibitors of Cell Division and Cytoskeleton

tubulin inhibitors, such as benzimidazoles, thiophanates: benomyl (D1.1), carbendazim (D1.2), fuberidazole (D1.3), thiabendazole (D1.4), thiophanate-methyl (D1.5); triazolopyrimidines: 5-chloro-7-(4-methylpiperidin-1-yl)-6-(2,4,6-trifluorophenyl)-[1,2,4]triazolo[1,5-a]pyrimidine (D1.6);

other cell division inhibitors: diethofencarb (D2.1), ethaboxam (D2.2), pencycuron (D2.3), fluopicolide (D2.4), zoxamide (D2.5), metrafenone (D2.6), pyriofenone (D2.7);

E) Inhibitors of Amino Acid and Protein Synthesis

methionine synthesis inhibitors (anilino-pyrimidines): cyprodinil (E.1.1), mepanipyrim (E.1.2), pyrimethanil (E.1.3);

protein synthesis inhibitors: blasticidin-S (E.2.1), kasugamycin (E.2.2), kasugamycin hydrochloride-hydrate (E.2.3), mildiomycin (E.2.4), streptomycin (E.2.5), oxytetracyclin (E.2.6), polyoxine (E.2.7), validamycin A (E.2.8);

F) Signal Transduction Inhibitors

MAP/histidine kinase inhibitors: fluoroimid (F.1.1), iprodione (F.1.2), procy-midone (F.1.3), vinclozolin (F.1.4), fenpiclonil (F.1.5), fludioxonil (F.1.6);

G protein inhibitors: quinoxyfen (F.2.1);

G) Lipid and Membrane Synthesis Ihibitors

Phospholipid biosynthesis inhibitors: edifenphos (G.1.1), iprobenfos (G.1.2), pyrazophos (G.1.3), isoprothiolane (G.1.4);

lipid peroxidation: dicloran (G.2.1), quintozene (G.2.2), tecnazene (G.2.3), tolclofos-methyl (G.2.4), biphenyl (G.2.5), chloroneb (G.2.6), etridiazole (G.2.7);

phospholipid biosynthesis and cell wall deposition: dimethomorph (G.3.1), flumorph (G.3.2), mandipropamid (G.3.3), pyrimorph (G.3.4), benthiavalicarb (G.3.5), iprovalicarb (G.3.6), valifenalate (G.3.7) and N-(1-(1-(4-cyano-phenyl)ethanesulfonyl)-but-2-yl) carbamic acid-(4-fluorophenyl) ester (G.3.8);

compounds affecting cell membrane permeability and fatty acides: propamocarb (G.4.1);

fatty acid amide hydrolase inhibitors: oxathiapiprolin (G.5.1), 2-{3-[2-(1-{[3,5-bis(di-fluoro-methyl-1H-pyrazol-1-yl]acetyl}piperidin-4-yl)-1,3-thiazol-4-yl]-4,5-dihydro-1,2-oxazol-5-yl}phenyl methanesulfonate (G.5.2), 2-{3-[2-(1-{[3,5-bis(difluoro-methyl)-1H-pyrazol-1-yl]acetyl}piperidin-4-yl) 1,3-thiazol-4-yl]-4,5-dihydro-1,2-oxazol-5-yl}-3-chlorophenyl methanesulfonate (G.5.3);

H) Inhibitors With Multi Site Action

inorganic active substances: Bordeaux mixture (H.1.1), copper acetate (H.1.2), copper hydroxide (H.1.3), copper oxychloride (H.1.4), basic copper sulfate (H.1.5), sulfur (H.1.6);

thio- and dithiocarbamates: ferbam (H.2.1), mancozeb (H.2.2), maneb (H.2.3), metam (H.2.4), metiram (H.2.5), propineb (H.2.6), thiram (H.2.7), zineb (H.2.8), ziram (H.2.9);

organochlorine compounds (e.g. phthalimides, sulfamides, chloronitriles): anilazine (H.3.1), chlorothalonil (H.3.2), captafol (H.3.3), captan (H.3.4), folpet (H.3.5), dichlofluanid (H.3.6), dichlorophen (H.3.7), hexachlorobenzene (H.3.8), pentachlorphenole (H.3.9) and its salts, phthalide (H.3.10), tolylfluanid (H.3.11), N-(4-chloro-2-nitro-phenyl)-N-ethyl-4-methyl-benzenesulfonamide (H.3.12);

guanidines and others: guanidine (H.4.1), dodine (H.4.2), dodine free base (H.4.3), guazatine (H.4.4), guazatine-acetate (H.4.5), iminoctadine (H.4.6), iminoctadine-triacetate (H.4.7), iminoctadine-tris(albesilate) (H.4.8), dithianon (H.4.9), 2,6-dimethyl-1H,5H-[1,4]dithiino[2,3-c:5,6-c′]dipyrrole-1,3,5,7(2H,6H)-tetraone (H.4.10);

I) Cell Wall Synthesis Inhibitors

inhibitors of glucan synthesis: validamycin (1.1.1), polyoxin B (1.1.2);

melanin synthesis inhibitors: pyroquilon (1.2.1), tricyclazole (1.2.2), carpropamid (1.2.3), dicyclomet (1.2.4), fenoxanil (1.2.5);

J) Plant Defence Inducers

acibenzolar-S-methyl (J.1.1), probenazole (J.1.2), isotianil (J.1.3), tiadinil (J.1.4), prohexadione-calcium (J.1.5); phosphonates: fosetyl (J.1.6), fosetyl-aluminum (J.1.7), phosphorous acid and its salts (J.1.8), potassium or sodium bicarbonate (J.1.9);

K) Unknown Mode of Action

bronopol (K.1.1), chino-methionat (K.1.2), cyflufenamid (K.1.3), cymoxanil (K.1.4), dazomet (K.1.5), debacarb (K.1.6), diclomezine (K.1.7), difenzoquat (K.1.8), difenzoquat-methylsulfate (K.1.9), diphenylamin (K.1.10), fenpyrazamine (K.1.11), flu-metover (K.1.12), flusulfamide (K.1.13), flutianil (K.1.14), methasulfocarb (K.1.15), nitrapyrin (K.1.16), nitrothal-isopropyl (K.1.18), oxathiapiprolin (K.1.19), tolprocarb (K.1.20), oxin-copper (K.1.21), proquinazid (K.1.22), tebufloquin (K.1.23), tecloftalam (K.1.24), triazoxide (K.1.25), 2-butoxy-6-iodo-3-propylchromen-4-one (K.1.26), 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone (K.1.27), 2-[3,5-bis(difluoro-methyl)-1H-pyrazol-1-yl]-1-[4-(4-{5-[2-fluoro-6-(prop-2-yn-1-yl-oxy)phenyl]-4,5-dihydro-1,2-oxazol-3-yl}-1,3-thiazol-2-yl)piperidin-1-yl]ethanone (K.1.28), 2-[3,5-bis(difluoro-methyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone (K.1.29), N-(cyclo-propyl-methoxyimino-(6-difluoro-methoxy-2,3-difluoro-phenyl)-methyl)-2-phenyl acetamide (K.1.30), N′-(4-(4-chloro-3-trifluoromethyl-phenoxy)-2,5-dimethyl-phenyl)-N-ethyl-N-methyl formamidine (K.1.31), N′-(4-(4-fluoro-3-trifluoromethyl-phenoxy)-2,5-dimethyl-phenyl)-N-ethyl-N-methyl formamidine (K.1.32), N′-(2-methyl-5-trifluoromethyl-4-(3-tri-methylsilanyl-propoxy)-phenyl)-N-ethyl-N-methyl formamidine (K.1.33), N′-(5-difluoromethyl-2-methyl-4-(3-trimethylsilanyl-propoxy)-phenyl)-N-ethyl-N-methyl formamidine (K.1.34), methoxy-acetic acid 6-tert-butyl-8-fluoro-2,3-dimethyl-quinolin-4-ylester (K.1.35), 3-[5-(4-methylphenyl)-2,3-dimethyl-isoxazolidin-3-yl]-pyridine (K.1.36), 3-[5-(4-chloro-phenyl)-2,3-dimethyl-isoxazolidin-3-yl]-pyridine (pyrisoxazole) (K.1.37), N-(6-methoxy-pyridin-3-yl) cyclopropanecarboxylic acid amide (K.1.38), 5-chloro-1-(4,6-dinnethoxy-pyri-midin-2-yl)-2-methyl-1H-benzoi-midazole (K.1.39), 2-(4-chloro-phenyl)-N-[4-(3,4-dinnethoxy-phenyl)-isoxazol-5-yl]-2-prop-2-ynyloxy-acetamide, ethyl (Z)-3-amino-2-cyano-3-phenyl-prop-2-enoate (K.1.40), picarbutrazox (K.1.41), pentyl N-[6-[[(Z)-[(1-methyltetrazol-5-yl)-phenyl-methylene]amino]oxy-methyl]-2-pyridyl]carbamate (K.1.42), 2-[2-[(7,8-difluoro-2-methyl-3-quinolyl)oxy]-6-fluoro-phenyl]propan-2-ol (K.1.43), 2-[2-fluoro-6-[(8-fluoro-2-methyl-3-quinolyl)oxy]phen-yl]propan-2-ol (K.1.44), 3-(5-fluoro-3,3,4,4-tetra-methyl-3,4-dihydroisoquinolin1-yl)quinoline (K.1.45), 3-(4,4-difluoro-3,3-dimethyl-3,4-dihydroisoquinolin-1-yl)quinoline (K.1.46), 3-(4,4,5-trifluoro-3,3-dimethyl-3,4-dihydroisoquinolin-1-yl)quinoline (K.1.47), 9-fluoro-2,2-dimethyl-5-(3-quinolyl)-3H-1,4-benzoxazepine (K.1.48);

M) Growth Regulators

abscisic acid (M.1.1), amidochlor, ancymidol, 6-benzylaminopurine, brassinolide, butralin, chlormequat, chlormequat chloride, choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid , maleic hydrazide, mefluidide, mepiquat, mepiquat chloride, naphthaleneacetic acid, N-6-benzyladenine, paclobutrazol, prohexadione, prohexadione-calcium, prohydrojasmon, thidiazuron, triapenthenol, tributyl phosphorotrithioate, 2,3,5-tri-iodobenzoic acid , trinexapac-ethyl and uniconazole;

N) Herbicides

acetamides: acetochlor (N.1.1), alachlor, butachlor, dimethachlor, dimethenamid (N.1.2), flufenacet (N.1.3), mefenacet (N.1.4), metolachlor (N.1.5), metazachlor (N.1.6), napropamide, naproanilide, pethoxamid, pretilachlor, propachlor, thenylchlor;

amino acid derivatives: bilanafos, glyphosate (N.2.1), glufosinate (N.2.2), sulfosate (N.2.3);

aryloxyphenoxypropionates: clodinafop (N.3.1), cyhalofop-butyl, fenoxaprop (N.3.2), fluazifop (N.3.3), haloxyfop (N.3.4), metamifop, propaquizafop, quizalofop, quizalofop-P-tefuryl;

Bipyridyls: diquat, paraquat (N.4.1);

(thio)carbamates: asulam, butylate, carbetamide, desmedipham, dimepiperate, eptam (EPTC), esprocarb, molinate, orbencarb, phenmedipham (N.5.1), prosulfocarb, pyributicarb, thiobencarb, triallate;

cyclohexanediones: butroxydim, clethodim (N.6.1), cycloxydim (N.6.2), profoxydim (N.6.3), sethoxydim (N.6.4), tepraloxydim (N.6.5), tralkoxydim;

dinitroanilines: benfluralin, ethalfluralin, oryzalin, pendimethalin (N.7.1), prodiamine (N.7.2), trifluralin (N.7.3);

diphenyl ethers: acifluorfen (N.8.1), aclonifen, bifenox, diclofop, ethoxyfen, fomesafen, lactofen, oxyfluorfen;

hydroxybenzonitriles: bomoxynil (N.9.1), dichlobenil, ioxynil;

imidazolinones: imazamethabenz, imazamox (N.10.1), imazapic (N.10.2), imazapyr (N.10.3), imazaquin (N.10.4), imazethapyr (N.10.5);

phenoxy acetic acids: clomeprop, 2,4-dichlorophenoxyacetic acid (2,4-D) (N.11.1), 2,4-DB, dichlorprop, MCPA, MCPA-thioethyl, MCPB, Mecoprop;

pyrazines: chloridazon (N.11.1), flufenpyr-ethyl, fluthiacet, norflurazon, pyridate;

pyridines: aminopyralid, clopyralid (N.12.1), diflufenican, dithiopyr, fluridone, fluroxypyr (N.12.2), picloram (N.12.3), picolinafen (N.12.4), thiazopyr;

sulfonyl ureas: amidosulfuron, azimsulfuron, bensulfuron (N.13.1), chlorimuron-ethyl (N.13.2), chlorsulfuron, cinosulfuron, cyclosulfamuron (N.13.3), ethoxysulfuron, flazasulfuron, flucetosulfuron, flupyrsulfuron, foramsulfuron, halosulfuron, imazosulfuron, iodosulfuron (N.13.4), mesosulfuron (N.13.5), metazosulfuron, metsulfuron-methyl (N.13.6), nicosulfuron (N.13.7), oxasulfuron, primisulfuron, prosulfuron, pyrazosulfuron, rimsulfuron (N.13.8), sulfometuron, sulfosulfuron, thifensulfuron, triasulfuron, tribenuron, trifloxysulfuron, triflusulfuron (N.13.9), tritosulfuron, 1-((2-chloro-6-propyl-i-midazo[1,2-b]pyridazin-3-yOsulfonyl)-3-(4,6-dimethoxy-pyrimidin-2-yl)urea;

triazines: ametryn, atrazine (N.14.1), cyanazine, dimethametryn, ethiozin, hexazinone (N.14.2), metamitron, metribuzin, prometryn, simazine, terbuthylazine, terbutryn, triaziflam, trifludimoxazin (N14.3);

ureas: chlorotoluron, daimuron, diuron (N.15.1), fluometuron, isoproturon, linuron, methabenzthiazuron, tebuthiuron;

other acetolactate synthase inhibitors: bispyribac-sodium, cloransulam-methyl, diclosulam, florasulam (N.16.1), flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone (N.16.2), pyroxsulam;

others: amicarbazone, aminotriazole, anilofos, beflubutamid, benazolin, bencarbazone, benfluresate, benzofenap, bentazone (N.17.1), benzobicyclon, bicyclopyrone, bromacil, bromobutide, butafenacil, butamifos, cafenstrole, carfentrazone, cinidon-ethyl (N.17.2), chlorthal, cinmethylin (N.17.3), clomazone (N.17.4), cumyluron, cyprosulfamide, dicamba (N.17.5), difenzoquat, diflufenzopyr (N.17.6), Drechslera monoceras, endothal, ethofumesate, etobenzanid, fenoxasulfone, fentrazamide, flumiclorac-pentyl, flumioxazin, flupoxam, flurochloridone, flurtamone, indanofan, isoxaben, isoxaflutole, lenacil, propanil, propyzamide, quinclorac (N.17.7), quinmerac (N.17.8), mesotrione (N.17.9), methyl arsonic acid, naptalam, oxadiargyl, oxadiazon, oxaziclomefone, pentoxazone, pinoxaden, pyraclonil, pyraflufen-ethyl, pyrasulfotole, pyrazoxyfen, pyrazolynate, quinoclamine, saflufenacil (N.17.10), sulcotrione (N.17.11), sulfentrazone, terbacil, tefuryltrione, tembotrione, thiencarbazone, topramezone (N.17.12), (3-[2-chloro-4-fluoro-5-(3-methyl-2,6-dioxo-4-trifluoromethyl-3,6-dihydro-2H-pyrimidin-1-yl)-phenoxy]-pyridin-2-yloxy)-acetic acid ethyl ester, 6-amino-5-chloro-2-cyclopropyl-pyrimidine-4-carboxylic acid methyl ester, 6-chloro-3-(2-cyclopropyl-6-methyl-phenoxy)-pyridazin-4-ol, 4-amino-3-chloro-6-(4-chloro-phenyl)-5-fluoro-pyridine-2-carboxylic acid, 4-amino-3-chloro-6-(4-chloro-2-fluoro-3-methoxy-phenyl)-pyridine-2-carboxylic acid methyl ester, and 4-amino-3-chloro-6-(4-chloro-3-di-methyla-mino-2-fluoro-phenyl)-pyridine-2-carboxylic acid methyl ester;

O) Insecticides

organo(thio)phosphates: acephate (O.1.1), azamethiphos (O.1.2), azinphos-methyl (O.1.3), chlorpyrifos (O.1.4), chlorpyrifos-methyl (O.1.5), chlorfenvinphos (O.1.6), diazinon (O.1.7), dichlorvos (O.1.8), dicrotophos (O.1.9), dimethoate (O.1.10), disulfoton (O.1.11), ethion (O.1.12), fenitrothion (O.1.13), fenthion (O.1.14), isoxathion (O.1.15), malathion (O.1.16), methamidophos (O.1.17), methidathion (O.1.18), methyl-parathion (O.1.19), mevinphos (O.1.20), monocrotophos (O.1.21), oxydemeton-methyl (O.1.22), paraoxon (O.1.23), parathion (O.1.24), phenthoate (O.1.25), phosalone (O.1.26), phosmet (O.1.27), phosphamidon (O.1.28), phorate (O.1.29), phoxim (O.1.30), pirimiphos-methyl (O.1.31), profenofos (O.1.32), prothiofos (O.1.33), sulprophos (O.1.34), tetrachlorvinphos (O.1.35), terbufos (O.1.36), triazophos (O.1.37), trichlorfon (O.1.38);

carbamates: alanycarb (O2.1), aldicarb (O2.2), bendiocarb (O2.3), benfuracarb (O2.4), carbaryl (O2.5), carbofuran (O2.6), carbosulfan (O2.7), fenoxycarb (O2.8), furathiocarb (O2.9), methiocarb (O2.10), methomyl (O2.11), oxamyl (O2.12), pirimicarb (O2.13), propoxur (O2.14), thiodicarb (O2.15), triazamate (O2.16);

pyrethroids: allethrin (O.3.1), bifenthrin (O.3.2), cyfluthrin (O.3.3), cyhalothrin (O.3.4), cyphenothrin (O.3.5), cypermethrin (O.3.6), alpha-cypermethrin (O.3.7), beta-cypermethrin (O.3.8), zeta-cypermethrin (O.3.9), deltamethrin (O.3.10), esfenvalerate (O.3.11), etofenprox (O.3.11), fenpropathrin (O.3.12), fenvalerate (O.3.13), imiprothrin (O.3.14), lambda-cyhalothrin (O.3.15), permethrin (O.3.16), prallethrin (O.3.17), pyrethrin I and II (O.3.18), resmethrin (O.3.19), silafluofen (O.3.20), tau-fluvalinate (O.3.21), tefluthrin (O.3.22), tetramethrin (O.3.23), tralomethrin (O.3.24), transfluthrin (O.3.25), profluthrin (O.3.26), dimefluthrin (O.3.27);

insect growth regulators: a) chitin synthesis inhibitors: benzoylureas: chlorfluazuron (0.4.1), cyramazin (0.4.2), diflubenzuron (0.4.3), flucycloxuron (0.4.4), flufenoxuron (0.4.5), hexaflumuron (0.4.6), lufenuron (0.4.7), novaluron (0.4.8), teflubenzuron (0.4.9), triflumuron (0.4.10); buprofezin (0.4.11), diofenolan (0.4.12), hexythiazox (0.4.13), etoxazole (0.4.14), clofentazine (0.4.15); b) ecdysone antagonists: halofenozide (0.4.16), methoxyfenozide (0.4.17), tebufenozide (0.4.18), azadirachtin (0.4.19); c) juvenoids: pyriproxyfen (0.4.20), methoprene (0.4.21), fenoxycarb (0.4.22); d) lipid biosynthesis inhibitors: spirodiclofen (0.4.23), spiromesifen (0.4.24), spirotetramat (0.4.24);

nicotinic receptor agonists/antagonists compounds: clothianidin (0.5.1), dinotefuran (0.5.2), flupyradifurone (0.5.3), imidacloprid (0.5.4), thiamethoxam (0.5.5), nitenpyram (0.5.6), acetamiprid (0.5.7), thiacloprid (0.5.8), 1-2-chloro-thiazol-5-ylmethyl)-2-nitrimino-3,5-dimethyl-[1,3,5]triazinane (0.5.9);

GABA antagonist compounds: endosulfan (0.6.19, ethiprole (0.6.2), fipronil (0.6.3), vaniliprole (0.6.4), pyrafluprole (0.6.5), pyriprole (0.6.6), 5-amino-1-(2,6-dichloro-4-methylphenyl)-4-sulfina-moyl-1H-pyrazole-3-carbothioic acid amide (0.6.7);

macrocyclic lactone insecticides: abamectin (0.7.1), emamectin (0.7.2), milbemectin (0.7.3), lepi-mectin (0.7.4), spinosad (0.7.5), spinetoram (0.7.6);

mitochondrial electron transport inhibitor (METI) I acaricides: fenazaquin (0.8.1), pyridaben (0.8.2), tebufenpyrad (0.8.3), tolfenpyrad (0.8.4), flufenerim (0.8.5);

METI II and III compounds: acequinocyl (0.9.1), fluacyprim (0.9.2), hydramethylnon (0.9.3);

Uncouplers: Chlorfenapyr (O.10.1);

oxidative phosphorylation inhibitors: cyhexatin (O.11.1), diafenthiuron (O.11.2), fenbutatin oxide (O.11.3), propargite (O.11.4);

moulting disruptor compounds: cryomazine (O.12.1);

mixed function oxidase inhibitors: piperonyl butoxide (O.13.1);

sodium channel blockers: indoxacarb (O.14.1), metaflumizone (O.14.2);

ryanodine receptor inhibitors: chlorantraniliprole (O.15.1), cyantraniliprole (O.15.2), flubendiamide (O.15.3), N-[4,6-dichloro-2-[(diethyl-lannbda-4-sulfanylidene)carbannoyl]-phenyl]-2-(3-chloro-2-pyridyl)-5-(trifluoro-methyl)pyrazole-3-carboxannide (O.15.4); N-[4-chloro-2-[(diethyl-lambda-4-sulfanylidene)carbannoyl]-6-methyl-phenyl]-2-(3-chloro-2-pyridyl)-5-(trifluoro-methyl)pyrazole-3-carboxamide (O.15.5); N-[4-chloro-2-[(di-2-propyl-lannbda-4-sulfanylidene)carbannoyl]-6-methyl-phenyl]-2-(3-chloro-2-pyridyl)-5-(trifluoro-methyl)pyrazole-3-carboxa-mide (O.15.6); N-[4,6-dichloro-2-[(di-2-propyl-lannbda-4-sulfanylidene)carbannoyl]-phenyl]-2-(3-chloro-2-pyridyl)-5-(trifluoromethyl)pyrazole-3-carboxannide (O.15.7); N-[4,6-dichloro-2-[(diethyl-lambda-4-sulfanylidene)carbannoyl]-phenyl]-2-(3-chloro-2-pyridyl)-5-(difluoro-methyl)pyrazole-3-carboxamide (O.15.8); N-[4,6-dibromo-2-[(di-2-propyl-lannbda-4-sulfanylidene)carbamoyl]-phenyl]-2-(3-chloro-2-pyridyl)-5-(trifluoro-methyl)pyrazole-3-carboxannide (O.15.9); N-[4-chloro-2-[(di-2-propyl-lambda-4-su lfanylidene)carbamoyl]-6-cyano-phenyl]-2-(3-chloro-2-pyridyl)-5-(trifluoromethyl)pyrazole-3-carboxamide (O.15.10); N-[4,6-dibromo-2-[(diethyl-lambda-4-sulfanylidene)carbamoyl]-phenyl]-2-(3-chloro-2-pyridyl)-5-(trifluoro-methyl)pyrazole-3-carboxamide (O.15.11);

others: benclothiaz (O.16.1), bifenazate (O.16.2), artap (O.16.3), flonicamid (O.16.4), pyridalyl (O.16.5), pymetrozine (O.16.6), sulfur (O.16.7), thiocyclam (O.16.8), cyenopyrafen (O.16.9), flupyrazofos (O.16.10), cyflumetofen (O.16.11), amidoflumet (O.16.12), imicyafos (O.16.13), bistrifluron (O.16.14), pyrifluquinazon (O.16.15),

1,1′-[(3S,4R,4aR,6S,6aS,12R,12aS,12bS)-4-[[(2-cyclopropylacetyl)oxy]-methyl]-1,3,4,4a,5,6,6a,12,12a,12b-decahydro-12-hydroxy-4,6a,12b-trimethyl-11-oxo-9-(3-pyridinyl)-2H,11H-naphtho[2,1-b]pyrano[3,4-e]pyran-3,6-diyl] cyclopropaneacetic acid ester (O.16.16), tioxazafen (016.7).

Preferred two-component compositions comprising a compound of formula I according to the present invention are compiled in the following Table D:

TABLE D
Individual preferred pesticidal compositions D-1 to D-432 comprising a particular
compound of formula I, namely one of I.Ka-1 to I.Ka-11 as component I (from Table C above)
and a particular second pesticidal compound as component II:

| | component I | |
|---|---|---|
| | compound | |
| | from Table C | |
| Composition | above | component II |
| D-1 | I.Ka-1 | Pyraclostrobin |
| D-2 | I.Ka-1 | Azoxystrobin |
| D-3 | I.Ka-1 | Trifloxystrobin |
| D-4 | I.Ka-1 | Picoxystrobin |
| D-5 | I.Ka-1 | Fluoxastrobin |
| D-6 | I.Ka-1 | Dimoxystrobin |
| D-7 | I.Ka-1 | Kresoxim-methyl |
| D-8 | I.Ka-1 | (2E,3Z)-5-[[1-(2,4-dichlorophenyl)-1H-pyrazol-3-yl]oxy]-2- |
| | | (methoxyimino)-N,3-dimethyl-pent-3-enamide |
| D-9 | I.Ka-1 | (2E,3Z)-5-[[1-(4-chlorophenyl)-1H-pyrazol-3-yl]oxy]-2- |
| | | (methoxyimino)-N,3-dimethyl-pent-3-enamide |
| D-10 | I.Ka-1 | [(3S,6S,7R,8R)-8-benzyl-3-[(3-acetoxy-4 methoxy-pyridine-2- |
| | | carbonyl)amino]-6-methyl-4,9-dioxo-1,5-dioxonan-7-yl] 2 |
| | | methylpropanoate |
| D-11 | I.Ka-1 | [(3S,6S,7R,8R)-8-benzyl-3-[[3-(acetoxymethoxy)-4-methoxy- |
| | | pyridine-2 carbonyl]amino]-6-methyl-4,9-dioxo-1,5-dioxonan-7-yl] 2 |
| | | methylpropanoate |
| D-12 | I.Ka-1 | [(3S,6S,7R,8R)-8-benzyl-3-[(3-isobutoxycarbonyloxy-4-methoxy- |
| | | pyridine-2 carbonyl)amino]-6-methyl-4,9-dioxo-1,5-dioxonan-7-yl] 2- |
| | | methylpropanoate |
| D-13 | I.Ka-1 | [(3S,6S,7R,8R)-8-benzyl-3-[[3-(1,3-benzodioxol-5-ylmethoxy)-4- |
| | | methoxy-pyridine-2-carbonyl]amino]-6-methyl-4,9-dioxo-1,5- |
| | | dioxonan-7-yl] 2-methylpropanoate |
| D-14 | I.Ka-1 | (3S,6S,7R,8R)-3-[[(3-hydroxy-4-methoxy-2- |
| | | pyridinyl)carbonyl]amino]-6 methyl-4,9-dioxo-8-(phenylmethyl)-1,5- |
| | | dioxonan-7-yl 2-methylpropanoate |
| D-15 | I.Ka-1 | Fluxapyroxad |
| D-16 | I.Ka-1 | Boscalid |
| D-17 | I.Ka-1 | Bixafen |
| D-18 | I.Ka-1 | Isopyrazam |
| D-19 | I.Ka-1 | Benzovindiflupyr |
| D-20 | I.Ka-1 | Fluopyram |
| D-21 | I.Ka-1 | N-[(5-chloro-2-isopropyl-phenyl)methyl]-N-cyclopropyl-5-fluoro-1,3- |
| | | dimethyl-pyrazole-4-carboxamide |
| D-22 | I.Ka-1 | Sedaxane |
| D-23 | I.Ka-1 | Penflufen |
| D-24 | I.Ka-1 | N-[2-(2,4-dichlorophenyl)-2-methoxy-1-methyl-ethyl]-3- |
| | | (difluoromethyl)-1-methyl-pyrazole-4-carboxamide |
| D-25 | I.Ka-1 | 3 (difluoromethyl)-1-methyl-N-(1,1,3-trimethylindan-4-yl)pyrazole-4- |
| | | carboxamide |
| D-26 | I.Ka-1 | 3 (trifluoromethyl)-1-methyl-N-(1,1,3-trimethylindan-4-yl)pyrazole-4- |
| | | carboxamide |
| D-27 | I.Ka-1 | 1,3-dimethyl-N-(1,1,3-trimethylindan-4-yl)pyrazole-4-carboxamide |
| D-28 | I.Ka-1 | 3-(trifluorometh¬yl)-1,5-dimethyl-N-(1,1,3-trimethylindan-4- |
| | | yl)pyrazole-4-carboxamide |
| D-29 | I.Ka-1 | 1,3,5-trimethyl-N-(1,1,3-trimethylindan-4-yl)pyrazole-4-carboxamide |
| D-30 | I.Ka-1 | 1-[3-chloro-2-[[[1-(4-chlorophenyl)-1H-pyrazol-3- |
| | | yl]oxy]methyl]phenyl]-1,4-dihydro-4-methyl-5H-tetrazol-5-one |
| D-31 | I.Ka-1 | Ametoctradin |
| D-32 | I.Ka-1 | epoxiconazole |
| D-33 | I.Ka-1 | metconazole |
| D-34 | I.Ka-1 | prothioconazole |
| D-35 | I.Ka-1 | difenoconazole |
| D-36 | I.Ka-1 | fluquinconazole |
| D-37 | I.Ka-1 | propiconazole |
| D-38 | I.Ka-1 | tebuconazole |
| D-39 | I.Ka-1 | 2-[2-chloro-4-(4-chlorophenoxy)phenyl]-1 (1,2,4-triazol-1-yl)pentan- |
| | | 2-ol |
| D-40 | I.Ka-1 | 1-[4-(4-chlorophenoxy)-2-(trifluoromethyl)phenyl]-1 cyclopropyl-2- |
| | | (1,2,4-triazol-1-yl)ethanol |
| D-41 | I.Ka-1 | 2-[4-(4-chlorophenoxy)-2-(trifluoromethyl)phenyl]-1-(1,2,4-triazol-1- |
| | | yl)butan-2-ol |
| D-42 | I.Ka-1 | 2-[2-chloro-4-(4-chlorophenoxy)phenyl]-1-(1,2,4-triazol-1-yl)butan- |
| | | 2-ol |
| D-43 | I.Ka-1 | 2-[4-(4-chlorophenoxy)-2-(trifluoromethyl)phenyl]-3-methyl-1-(1,2,4- |
| | | triazol-1-yl)butan-2-ol |
| D-44 | I.Ka-1 | 2-[4-(4-chlorophenoxy)-2-(trifluoromethyl)phenyl]-1-(1,2,4-triazol-1- |
| | | yl)propan-2-ol |
| D-45 | I.Ka-1 | 2-[2-chloro-4-(4-chlorophenoxy)phenyl]-3-methyl-1-(1,2,4-triazol-1- |
| | | yl)butan-2-ol |
| D-46 | I.Ka-1 | 2-[4-(4-chlorophenoxy)-2-(trifluoromethyl)phenyl]-1-(1,2,4-triazol-1- |
| | | yl)pentan-2-ol |
| D-47 | I.Ka-1 | 2-[4-(4-fluorophenoxy)-2-(trifluoromethyl)phenyl]-1-(1,2,4-triazol-1- |
| | | yl)propan-2-ol |
| D-48 | I.Ka-1 | 2-[2-chloro-4-(4-chlorophenoxy)phenyl]-1-(1,2,4-triazol-1-yl)pent-3- |
| | | yn-2-ol |
| D-49 | I.Ka-1 | prochloraz |
| D-50 | I.Ka-1 | [3-(4-chloro-2-fluoro-phenyl)-5-(2,4-difluorophenyl)isoxazol-4-yl]-(3- |
| | | pyridyl)methanol |
| D-51 | I.Ka-1 | fenpropimorph |
| D-52 | I.Ka-1 | Metalaxyl |
| D-53 | I.Ka-1 | Benalaxyl |
| D-54 | I.Ka-1 | Thiophanate-methyl |
| D-55 | I.Ka-1 | Carbendazim |
| D-56 | I.Ka-1 | Metrafenone |
| D-57 | I.Ka-1 | Pyrimethanil |
| D-58 | I.Ka-1 | Iprodione |
| D-59 | I.Ka-1 | Vinclozolin |
| D-60 | I.Ka-1 | Fludioxonil |
| D-61 | I.Ka-1 | dimethomorph |
| D-62 | I.Ka-1 | oxathiapiprolin |
| D-63 | I.Ka-1 | metiram |
| D-64 | I.Ka-1 | mancozeb |
| D-65 | I.Ka-1 | chlorothalonil |
| D-66 | I.Ka-1 | dithianon |
| D-67 | I.Ka-1 | Dipymetitrone |
| D-68 | I.Ka-1 | prohexadione-calcium |
| D-69 | I.Ka-1 | 3-(5-fluoro-3,3,4,4-tetramethyl-3,4-dihydroisoquinolin-1-yl)quinoline |
| D-70 | I.Ka-1 | 3-(4,4-difluoro-3,3-dimethyl-3,4-dihydroisoquinolin-1-yl)quinoline |
| D-71 | I.Ka-1 | 3-(4,4,5-trifluoro-3,3-dimethyl-3,4-dihydroisoquinolin-1-yl)quinoline |
| D-72 | I.Ka-1 | 9-fluoro-2,2-dimethyl-5-(3-quinolyl)-3H 1,4-benzoxazepine |
| D-73 | I.Ka-2 | Pyraclostrobin |
| D-74 | I.Ka-2 | Azoxystrobin |
| D-75 | I.Ka-2 | Trifloxystrobin |
| D-76 | I.Ka-2 | Picoxystrobin |
| D-77 | I.Ka-2 | Fluoxastrobin |
| D-78 | I.Ka-2 | Dimoxystrobin |
| D-79 | I.Ka-2 | Kresoxim-methyl |
| D-80 | I.Ka-2 | (2E,3Z)-5-[[1-(2,4-dichlorophenyl)-1H-pyrazol-3-yl]oxy]-2- |
| | | (methoxyimino)-N,3-dimethyl-pent-3-enamide |
| D-81 | I.Ka-2 | (2E,3Z)-5-[[1-(4-chlorophenyl)-1H-pyrazol-3-yl]oxy]-2- |
| | | (methoxyimino)-N,3-dimethyl-pent-3-enamide |
| D-82 | I.Ka-2 | [(3S,6S,7R,8R)-8-benzyl-3-[(3-acetoxy-4 methoxy-pyridine-2- |
| | | carbonyl)amino]-6-methyl-4,9-dioxo-1,5-dioxonan-7-yl] 2 |
| | | methylpropanoate |
| D-83 | I.Ka-2 | [(3S,6S,7R,8R)-8-benzyl-3-[[3-(acetoxymethoxy)-4-methoxy- |
| | | pyridine-2 carbonyl]amino]-6-methyl-4,9-dioxo-1,5-dioxonan-7-yl] 2 |
| | | methylpropanoate |
| D-84 | I.Ka-2 | [(3S,6S,7R,8R)-8-benzyl-3-[(3-isobutoxycarbonyloxy-4-methoxy- |
| | | pyridine-2 carbonyl)amino]-6-methyl-4,9-dioxo-1,5-dioxonan-7-yl] 2- |
| | | methylpropanoate |
| D-85 | I.Ka-2 | [(3S,6S,7R,8R)-8-benzyl-3-[[3-(1,3-benzodioxol-5-ylmethoxy)-4- |
| | | methoxy-pyridine-2-carbonyl]amino]-6-methyl-4,9-dioxo-1,5- |
| | | dioxonan-7-yl] 2-methylpropanoate |
| D-86 | I.Ka-2 | (3S,6S,7R,8R)-3-[[(3-hydroxy-4-methoxy-2- |
| | | pyridinyl)carbonyl]amino]-6 methyl-4,9-dioxo-8-(phenylmethyl)-1,5- |
| | | dioxonan-7-yl 2-methylpropanoate |
| D-87 | I.Ka-2 | Fluxapyroxad |
| D-88 | I.Ka-2 | Boscalid |
| D-89 | I.Ka-2 | Bixafen |
| D-90 | I.Ka-2 | Isopyrazam |
| D-91 | I.Ka-2 | Benzovindiflupyr |
| D-92 | I.Ka-2 | Fluopyram |
| D-93 | I.Ka-2 | N-[(5-chloro-2-isopropyl-phenyl)methyl]-N-cyclopropyl-5-fluoro-1,3- |
| | | dimethyl-pyrazole-4-carboxamide |
| D-94 | I.Ka-2 | Sedaxane |
| D-95 | I.Ka-2 | Penflufen |
| D-96 | I.Ka-2 | N-[2-(2,4-dichlorophenyl)-2-methoxy-1-methyl-ethyl]-3- |
| | | (difluoromethyl)-1-methyl-pyrazole-4-carboxamide |
| D-97 | I.Ka-2 | 3 (difluoromethyl)-1-methyl-N-(1,1,3-trimethylindan-4-yl)pyrazole-4- |
| | | carboxamide |
| D-98 | I.Ka-2 | 3 (trifluoromethyl)-1-methyl-N-(1,1,3-trimethylindan-4-yl)pyrazole-4- |
| | | carboxamide |
| D-99 | I.Ka-2 | 1,3-dimethyl-N-(1,1,3-trimethylindan-4-yl)pyrazole-4-carboxamide |
| D-100 | I.Ka-2 | 3-(trifluorometh¬yl)-1,5-dimethyl-N-(1,1,3-trimethylindan-4- |
| | | yl)pyrazole-4-carboxamide |
| D-101 | I.Ka-2 | 1,3,5-trimethyl-N-(1,1,3-trimethylindan-4-yl)pyrazole-4-carboxamide |
| D-102 | I.Ka-2 | 1-[3-chloro-2-[[[1-(4-chlorophenyl)-1H-pyrazol-3- |
| | | yl]oxy]methyl]phenyl]-1,4-dihydro-4-methyl-5H-tetrazol-5-one |
| D-103 | I.Ka-2 | Ametoctradin |
| D-104 | I.Ka-2 | epoxiconazole |
| D-105 | I.Ka-2 | metconazole |
| D-106 | I.Ka-2 | prothioconazole |
| D-107 | I.Ka-2 | difenoconazole |
| D-108 | I.Ka-2 | fluquinconazole |
| D-109 | I.Ka-2 | propiconazole |
| D-110 | I.Ka-2 | tebuconazole |
| D-111 | I.Ka-2 | 2-[2-chloro-4-(4-chlorophenoxy)phenyl]-1 (1,2,4-triazol-1-yl)pentan- |
| | | 2-ol |
| D-112 | I.Ka-2 | 1-[4-(4-chlorophenoxy)-2-(trifluoromethyl)phenyl]-1 cyclopropyl-2- |
| | | (1,2,4-triazol-1-yl)ethanol |
| D-113 | I.Ka-2 | 2-[4-(4-chlorophenoxy)-2-(trifluoromethyl)phenyl]-1-(1,2,4-triazol-1- |
| | | yl)butan-2-ol |
| D-114 | I.Ka-2 | 2-[2-chloro-4-(4-chlorophenoxy)phenyl]-1-(1,2,4-triazol-1-yl)butan- |
| | | 2-ol |
| D-115 | I.Ka-2 | 2-[4-(4-chlorophenoxy)-2-(trifluoromethyl)phenyl]-3-methyl-1-(1,2,4- |
| | | triazol-1-yl)butan-2-ol |
| D-116 | I.Ka-2 | 2-[4-(4-chlorophenoxy)-2-(trifluoromethyl)phenyl]-1-(1,2,4-triazol-1- |
| | | yl)propan-2-ol |
| D-117 | I.Ka-2 | 2-[2-chloro-4-(4-chlorophenoxy)phenyl]-3-methyl-1-(1,2,4-triazol-1- |
| | | yl)butan-2-ol |
| D-118 | I.Ka-2 | 2-[4-(4-chlorophenoxy)-2-(trifluoromethyl)phenyl]-1-(1,2,4-triazol-1- |
| | | yl)pentan-2-ol |
| D-119 | I.Ka-2 | 2-[4-(4-fluorophenoxy)-2-(trifluoromethyl)phenyl]-1-(1,2,4-triazol-1- |
| | | yl)propan-2-ol |
| D-120 | I.Ka-2 | 2-[2-chloro-4-(4-chlorophenoxy)phenyl]-1-(1,2,4-triazol-1-yl)pent-3- |
| | | yn-2-ol |
| D-121 | I.Ka-2 | prochloraz |
| D-122 | I.Ka-2 | [3-(4-chloro-2-fluoro-phenyl)-5-(2,4-difluorophenyl)isoxazol-4-yl]-(3- |
| | | pyridyl)methanol |
| D-123 | I.Ka-2 | fenpropimorph |
| D-124 | I.Ka-2 | Metalaxyl |
| D-125 | I.Ka-2 | Benalaxyl |
| D-126 | I.Ka-2 | Thiophanate-methyl |
| D-127 | I.Ka-2 | Carbendazim |
| D-128 | I.Ka-2 | Metrafenone |
| D-129 | I.Ka-2 | Pyrimethanil |
| D-130 | I.Ka-2 | Iprodione |
| D-131 | I.Ka-2 | Vinclozolin |
| D-132 | I.Ka-2 | Fludioxonil |
| D-133 | I.Ka-2 | dimethomorph |
| D-134 | I.Ka-2 | oxathiapiprolin |
| D-135 | I.Ka-2 | metiram |
| D-136 | I.Ka-2 | mancozeb |
| D-137 | I.Ka-2 | chlorothalonil |
| D-138 | I.Ka-2 | dithianon |
| D-139 | I.Ka-2 | Dipymetitrone |
| D-140 | I.Ka-2 | prohexadione-calcium |
| D-141 | I.Ka-2 | 3-(5-fluoro-3,3,4,4-tetramethyl-3,4-dihydroisoquinolin-1-yl)quinoline |
| D-142 | I.Ka-2 | 3-(4,4-difluoro-3,3-dimethyl-3,4-dihydroisoquinolin-1-yl)quinoline |
| D-143 | I.Ka-2 | 3-(4,4,5-trifluoro-3,3-dimethyl-3,4-dihydroisoquinolin-1-yl)quinoline |
| D-144 | I.Ka-2 | 9-fluoro-2,2-dimethyl-5-(3-quinolyl)-3H 1,4-benzoxazepine |
| D-145 | I.Ka-3 | Pyraclostrobin |
| D-146 | I.Ka-3 | Azoxystrobin |
| D-147 | I.Ka-3 | Trifloxystrobin |
| D-148 | I.Ka-3 | Picoxystrobin |
| D-149 | I.Ka-3 | Fluoxastrobin |
| D-150 | I.Ka-3 | Dimoxystrobin |
| D-151 | I.Ka-3 | Kresoxim-methyl |
| D-152 | I.Ka-3 | (2E,3Z)-5-[[1-(2,4-dichlorophenyl)-1H-pyrazol-3-yl]oxy]-2- |
| | | (methoxyimino)-N,3-dimethyl-pent-3-enamide |
| D-153 | I.Ka-3 | (2E,3Z)-5-[[1-(4-chlorophenyl)-1H-pyrazol-3-yl]oxy]-2- |
| | | (methoxyimino)-N,3-dimethyl-pent-3-enamide |
| D-154 | I.Ka-3 | [(3S,6S,7R,8R)-8-benzyl-3-[(3-acetoxy-4 methoxy-pyridine-2- |
| | | carbonyl)amino]-6-methyl-4,9-dioxo-1,5-dioxonan-7-yl] 2 |
| | | methylpropanoate |
| D-155 | I.Ka-3 | [(3S,6S,7R,8R)-8-benzyl-3-[[3-(acetoxymethoxy)-4-methoxy- |
| | | pyridine-2 carbonyl]amino]-6-methyl-4,9-dioxo-1,5-dioxonan-7-yl] 2 |
| | | methylpropanoate |
| D-156 | I.Ka-3 | [(3S,6S,7R,8R)-8-benzyl-3-[(3-isobutoxycarbonyloxy-4-methoxy- |
| | | pyridine-2 carbonyl)amino]-6-methyl-4,9-dioxo-1,5-dioxonan-7-yl] 2- |
| | | methylpropanoate |
| D-157 | I.Ka-3 | [(3S,6S,7R,8R)-8-benzyl-3-[[3-(1,3-benzodioxol-5-ylmethoxy)-4- |
| | | methoxy-pyridine-2-carbonyl]amino]-6-methyl-4,9-dioxo-1,5- |
| | | dioxonan-7-yl] 2-methylpropanoate |
| D-158 | I.Ka-3 | (3S,6S,7R,8R)-3-[[(3-hydroxy-4-methoxy-2- |
| | | pyridinyl)carbonyl]amino]-6 methyl-4,9-dioxo-8-(phenylmethyl)-1,5- |
| | | dioxonan-7-yl 2-methylpropanoate |
| D-159 | I.Ka-3 | Fluxapyroxad |
| D-160 | I.Ka-3 | Boscalid |
| D-161 | I.Ka-3 | Bixafen |
| D-162 | I.Ka-3 | Isopyrazam |
| D-163 | I.Ka-3 | Benzovindiflupyr |
| D-164 | I.Ka-3 | Fluopyram |
| D-165 | I.Ka-3 | N-[(5-chloro-2-isopropyl-phenyl)methyl]-N-cyclopropyl-5-fluoro-1,3- |
| | | dimethyl-pyrazole-4-carboxamide |
| D-166 | I.Ka-3 | Sedaxane |
| D-167 | I.Ka-3 | Penflufen |
| D-168 | I.Ka-3 | N-[2-(2,4-dichlorophenyl)-2-methoxy-1-methyl-ethyl]-3- |
| | | (difluoromethyl)-1-methyl-pyrazole-4-carboxamide |
| D-169 | I.Ka-3 | 3 (difluoromethyl)-1-methyl-N-(1,1,3-trimethylindan-4-yl)pyrazole-4- |
| | | carboxamide |
| D-170 | I.Ka-3 | 3 (trifluoromethyl)-1-methyl-N-(1,1,3-trimethylindan-4-yl)pyrazole-4- |
| | | carboxamide |
| D-171 | I.Ka-3 | 1,3-dimethyl-N-(1,1,3-trimethylindan-4-yl)pyrazole-4-carboxamide |
| D-172 | I.Ka-3 | 3-(trifluorometh¬yl)-1,5-dimethyl-N-(1,1,3-trimethylindan-4- |
| | | yl)pyrazole-4-carboxamide |
| D-173 | I.Ka-3 | 1,3,5-trimethyl-N-(1,1,3-trimethylindan-4-yl)pyrazole-4-carboxamide |
| D-174 | I.Ka-3 | 1-[3-chloro-2-[[[1-(4-chlorophenyl)-1H-pyrazol-3- |
| | | yl]oxy]methyl]phenyl]-1,4-dihydro-4-methyl-5H-tetrazol-5-one |
| D-175 | I.Ka-3 | Ametoctradin |
| D-176 | I.Ka-3 | epoxiconazole |
| D-177 | I.Ka-3 | metconazole |
| D-178 | I.Ka-3 | prothioconazole |
| D-179 | I.Ka-3 | difenoconazole |
| D-180 | I.Ka-3 | fluquinconazole |
| D-181 | I.Ka-3 | propiconazole |
| D-182 | I.Ka-3 | tebuconazole |
| D-183 | I.Ka-3 | 2-[2-chloro-4-(4-chlorophenoxy)phenyl]-1 (1,2,4-triazol-1-yl)pentan- |
| | | 2-ol |
| D-184 | I.Ka-3 | 1-[4-(4-chlorophenoxy)-2-(trifluoromethyl)phenyl]-1 cyclopropyl-2- |
| | | (1,2,4-triazol-1-yl)ethanol |
| D-185 | I.Ka-3 | 2-[4-(4-chlorophenoxy)-2-(trifluoromethyl)phenyl]-1-(1,2,4-triazol-1- |
| | | yl)butan-2-ol |
| D-186 | I.Ka-3 | 2-[2-chloro-4-(4-chlorophenoxy)phenyl]-1-(1,2,4-triazol-1-yl)butan- |
| | | 2-ol |
| D-187 | I.Ka-3 | 2-[4-(4-chlorophenoxy)-2-(trifluoromethyl)phenyl]-3-methyl-1-(1,2,4- |
| | | triazol-1-yl)butan-2-ol |
| D-188 | I.Ka-3 | 2-[4-(4-chlorophenoxy)-2-(trifluoromethyl)phenyl]-1-(1,2,4-triazol-1- |
| | | yl)propan-2-ol |
| D-189 | I.Ka-3 | 2-[2-chloro-4-(4-chlorophenoxy)phenyl]-3-methyl-1-(1,2,4-triazol-1- |
| | | yl)butan-2-ol |
| D-190 | I.Ka-3 | 2-[4-(4-chlorophenoxy)-2-(trifluoromethyl)phenyl]-1-(1,2,4-triazol-1- |
| | | yl)pentan-2-ol |
| D-191 | I.Ka-3 | 2-[4-(4-fluorophenoxy)-2-(trifluoromethyl)phenyl]-1-(1,2,4-triazol-1- |
| | | yl)propan-2-ol |
| D-192 | I.Ka-3 | 2-[2-chloro-4-(4-chlorophenoxy)phenyl]-1-(1,2,4-triazol-1-yl)pent-3- |
| | | yn-2-ol |
| D-193 | I.Ka-3 | prochloraz |
| D-194 | I.Ka-3 | [3-(4-chloro-2-fluoro-phenyl)-5-(2,4-difluorophenyl)isoxazol-4-yl]-(3- |
| | | pyridyl)methanol |
| D-195 | I.Ka-3 | fenpropimorph |
| D-196 | I.Ka-3 | Metalaxyl |
| D-197 | I.Ka-3 | Benalaxyl |
| D-198 | I.Ka-3 | Thiophanate-methyl |
| D-199 | I.Ka-3 | Carbendazim |
| D-200 | I.Ka-3 | Metrafenone |
| D-201 | I.Ka-3 | Pyrimethanil |
| D-202 | I.Ka-3 | Iprodione |
| D-203 | I.Ka-3 | Vinclozolin |
| D-204 | I.Ka-3 | Fludioxonil |
| D-205 | I.Ka-3 | dimethomorph |
| D-206 | I.Ka-3 | oxathiapiprolin |
| D-207 | I.Ka-3 | metiram |
| D-208 | I.Ka-3 | mancozeb |
| D-209 | I.Ka-3 | chlorothalonil |
| D-210 | I.Ka-3 | dithianon |
| D-211 | I.Ka-3 | Dipymetitrone |
| D-212 | I.Ka-3 | prohexadione-calcium |
| D-213 | I.Ka-3 | 3-(5-fluoro-3,3,4,4-tetramethyl-3,4-dihydroisoquinolin-1-yl)quinoline |
| D-214 | I.Ka-3 | 3-(4,4-difluoro-3,3-dimethyl-3,4-dihydroisoquinolin-1-yl)quinoline |
| D-215 | I.Ka-3 | 3-(4,4,5-trifluoro-3,3-dimethyl-3,4-dihydroisoquinolin-1-yl)quinoline |
| D-216 | I.Ka-3 | 9-fluoro-2,2-dimethyl-5-(3-quinolyl)-3H 1,4-benzoxazepine |
| D-217 | I.Ka-4 | Pyraclostrobin |
| D-218 | I.Ka-4 | Azoxystrobin |
| D-219 | I.Ka-4 | Trifloxystrobin |
| D-220 | I.Ka-4 | Picoxystrobin |
| D-221 | I.Ka-4 | Fluoxastrobin |
| D-222 | I.Ka-4 | Dimoxystrobin |
| D-223 | I.Ka-4 | Kresoxim-methyl |
| D-224 | I.Ka-4 | (2E,3Z)-5-[[1-(2,4-dichlorophenyl)-1H-pyrazol-3-yl]oxy]-2- |
| | | (methoxyimino)-N,3-dimethyl-pent-3-enamide |
| D-225 | I.Ka-4 | (2E,3Z)-5-[[1-(4-chlorophenyl)-1H-pyrazol-3-yl]oxy]-2- |
| | | (methoxyimino)-N,3-dimethyl-pent-3-enamide |
| D-226 | I.Ka-4 | [(3S,6S,7R,8R)-8-benzyl-3-[(3-acetoxy-4 methoxy-pyridine-2- |
| | | carbonyl)amino]-6-methyl-4,9-dioxo-1,5-dioxonan-7-yl] 2 |
| | | methylpropanoate |
| D-227 | I.Ka-4 | [(3S,6S,7R,8R)-8-benzyl-3-[[3-(acetoxymethoxy)-4-methoxy- |
| | | pyridine-2 carbonyl]amino]-6-methyl-4,9-dioxo-1,5-dioxonan-7-yl] 2 |
| | | methylpropanoate |
| D-228 | I.Ka-4 | [(3S,6S,7R,8R)-8-benzyl-3-[(3-isobutoxycarbonyloxy-4-methoxy- |
| | | pyridine-2 carbonyl)amino]-6-methyl-4,9-dioxo-1,5-dioxonan-7-yl] 2- |
| | | methylpropanoate |
| D-229 | I.Ka-4 | [(3S,6S,7R,8R)-8-benzyl-3-[[3-(1,3-benzodioxol-5-ylmethoxy)-4- |
| | | methoxy-pyridine-2-carbonyl]amino]-6-methyl-4,9-dioxo-1,5- |
| | | dioxonan-7-yl] 2-methylpropanoate |
| D-230 | I.Ka-4 | (3S,6S,7R,8R)-3-[[(3-hydroxy-4-methoxy-2- |
| | | pyridinyl)carbonyl]amino]-6 methyl-4,9-dioxo-8-(phenylmethyl)-1,5- |
| | | dioxonan-7-yl 2-methylpropanoate |
| D-231 | I.Ka-4 | Fluxapyroxad |
| D-232 | I.Ka-4 | Boscalid |
| D-233 | I.Ka-4 | Bixafen |
| D-234 | I.Ka-4 | Isopyrazam |
| D-235 | I.Ka-4 | Benzovindiflupyr |
| D-236 | I.Ka-4 | Fluopyram |
| D-237 | I.Ka-4 | N-[(5-chloro-2-isopropyl-phenyl)methyl]-N-cyclopropyl-5-fluoro-1,3- |
| | | dimethyl-pyrazole-4-carboxamide |
| D-238 | I.Ka-4 | Sedaxane |
| D-239 | I.Ka-4 | Penflufen |
| D-240 | I.Ka-4 | N-[2-(2,4-dichlorophenyl)-2-methoxy-1-methyl-ethyl]-3- |
| | | (difluoromethyl)-1-methyl-pyrazole-4-carboxamide |
| D-241 | I.Ka-4 | 3 (difluoromethyl)-1-methyl-N-(1,1,3-trimethylindan-4-yl)pyrazole-4- |
| | | carboxamide |
| D-242 | I.Ka-4 | 3 (trifluoromethyl)-1-methyl-N-(1,1,3-trimethylindan-4-yl)pyrazole-4- |
| | | carboxamide |
| D-243 | I.Ka-4 | 1,3-dimethyl-N-(1,1,3-trimethylindan-4-yl)pyrazole-4-carboxamide |
| D-244 | I.Ka-4 | 3-(trifluorometh¬yl)-1,5-dimethyl-N-(1,1,3-trimethylindan-4- |
| | | yl)pyrazole-4-carboxamide |
| D-245 | I.Ka-4 | 1,3,5-trimethyl-N-(1,1,3-trimethylindan-4-yl)pyrazole-4-carboxamide |
| D-246 | I.Ka-4 | 1-[3-chloro-2-[[[1-(4-chlorophenyl)-1H-pyrazol-3- |
| | | yl]oxy]methyl]phenyl]-1,4-dihydro-4-methyl-5H-tetrazol-5-one |
| D-247 | I.Ka-4 | Ametoctradin |
| D-248 | I.Ka-4 | epoxiconazole |
| D-249 | I.Ka-4 | metconazole |
| D-250 | I.Ka-4 | prothioconazole |
| D-251 | I.Ka-4 | difenoconazole |
| D-252 | I.Ka-4 | fluquinconazole |
| D-253 | I.Ka-4 | propiconazole |
| D-254 | I.Ka-4 | tebuconazole |
| D-255 | I.Ka-4 | 2-[2-chloro-4-(4-chlorophenoxy)phenyl]-1 (1,2,4-triazol-1-yl)pentan- |
| | | 2-ol |
| D-256 | I.Ka-4 | 1-[4-(4-chlorophenoxy)-2-(trifluoromethyl)phenyl]-1 cyclopropyl-2- |
| | | (1,2,4-triazol-1-yl)ethanol |
| D-257 | I.Ka-4 | 2-[4-(4-chlorophenoxy)-2-(trifluoromethyl)phenyl]-1-(1,2,4-triazol-1- |
| | | yl)butan-2-ol |
| D-258 | I.Ka-4 | 2-[2-chloro-4-(4-chlorophenoxy)phenyl]-1-(1,2,4-triazol-1-yl)butan- |
| | | 2-ol |
| D-259 | I.Ka-4 | 2-[4-(4-chlorophenoxy)-2-(trifluoromethyl)phenyl]-3-methyl-1-(1,2,4- |
| | | triazol-1-yl)butan-2-ol |
| D-260 | I.Ka-4 | 2-[4-(4-chlorophenoxy)-2-(trifluoromethyl)phenyl]-1-(1,2,4-triazol-1- |
| | | yl)propan-2-ol |
| D-261 | I.Ka-4 | 2-[2-chloro-4-(4-chlorophenoxy)phenyl]-3-methyl-1-(1,2,4-triazol-1- |
| | | yl)butan-2-ol |
| D-262 | I.Ka-4 | 2-[4-(4-chlorophenoxy)-2-(trifluoromethyl)phenyl]-1-(1,2,4-triazol-1- |
| | | yl)pentan-2-ol |
| D-263 | I.Ka-4 | 2-[4-(4-fluorophenoxy)-2-(trifluoromethyl)phenyl]-1-(1,2,4-triazol-1- |
| | | yl)propan-2-ol |
| D-264 | I.Ka-4 | 2-[2-chloro-4-(4-chlorophenoxy)phenyl]-1-(1,2,4-triazol-1-yl)pent-3- |
| | | yn-2-ol |
| D-265 | I.Ka-4 | prochloraz |
| D-266 | I.Ka-4 | [3-(4-chloro-2-fluoro-phenyl)-5-(2,4-difluorophenyl)isoxazol-4-yl]-(3- |
| | | pyridyl)methanol |
| D-267 | I.Ka-4 | fenpropimorph |
| D-268 | I.Ka-4 | Metalaxyl |
| D-269 | I.Ka-4 | Benalaxyl |
| D-270 | I.Ka-4 | Thiophanate-methyl |
| D-271 | I.Ka-4 | Carbendazim |
| D-272 | I.Ka-4 | Metrafenone |
| D-273 | I.Ka-4 | Pyrimethanil |
| D-274 | I.Ka-4 | Iprodione |
| D-275 | I.Ka-4 | Vinclozolin |
| D-276 | I.Ka-4 | Fludioxonil |
| D-277 | I.Ka-4 | dimethomorph |
| D-278 | I.Ka-4 | oxathiapiprolin |
| D-279 | I.Ka-4 | metiram |
| D-280 | I.Ka-4 | mancozeb |
| D-281 | I.Ka-4 | chlorothalonil |
| D-282 | I.Ka-4 | dithianon |
| D-283 | I.Ka-4 | Dipymetitrone |
| D-284 | I.Ka-4 | prohexadione-calcium |
| D-285 | I.Ka-4 | 3-(5-fluoro-3,3,4,4-tetramethyl-3,4-dihydroisoquinolin-1-yl)quinoline |
| D-286 | I.Ka-4 | 3-(4,4-difluoro-3,3-dimethyl-3,4-dihydroisoquinolin-1-yl)quinoline |
| D-287 | I.Ka-4 | 3-(4,4,5-trifluoro-3,3-dimethyl-3,4-dihydroisoquinolin-1-yl)quinoline |
| D-288 | I.Ka-4 | 9-fluoro-2,2-dimethyl-5-(3-quinolyl)-3H 1,4-benzoxazepine |
| D-289 | I.Ka-5 | Pyraclostrobin |
| D-290 | I.Ka-5 | Azoxystrobin |
| D-291 | I.Ka-5 | Trifloxystrobin |
| D-292 | I.Ka-5 | Picoxystrobin |
| D-293 | I.Ka-5 | Fluoxastrobin |
| D-294 | I.Ka-5 | Dimoxystrobin |
| D-295 | I.Ka-5 | Kresoxim-methyl |
| D-296 | I.Ka-5 | (2E,3Z)-5-[[1-(2,4-dichlorophenyl)-1H-pyrazol-3-yl]oxy]-2- |
| | | (methoxyimino)-N,3-dimethyl-pent-3-enamide |
| D-297 | I.Ka-5 | (2E,3Z)-5-[[1-(4-chlorophenyl)-1H-pyrazol-3-yl]oxy]-2- |
| | | (methoxyimino)-N,3-dimethyl-pent-3-enamide |
| D-298 | I.Ka-5 | [(3S,6S,7R,8R)-8-benzyl-3-[(3-acetoxy-4 methoxy-pyridine-2- |
| | | carbonyl)amino]-6-methyl-4,9-dioxo-1,5-dioxonan-7-yl] 2 |
| | | methylpropanoate |
| D-299 | I.Ka-5 | [(3S,6S,7R,8R)-8-benzyl-3-[[3-(acetoxymethoxy)-4-methoxy- |
| | | pyridine-2 carbonyl]amino]-6-methyl-4,9-dioxo-1,5-dioxonan-7-yl] 2 |
| | | methylpropanoate |
| D-300 | I.Ka-5 | [(3S,6S,7R,8R)-8-benzyl-3-[(3-isobutoxycarbonyloxy-4-methoxy- |
| | | pyridine-2 carbonyl)amino]-6-methyl-4,9-dioxo-1,5-dioxonan-7-yl] 2- |
| | | methylpropanoate |
| D-301 | I.Ka-5 | [(3S,6S,7R,8R)-8-benzyl-3-[[3-(1,3-benzodioxol-5-ylmethoxy)-4- |
| | | methoxy-pyridine-2-carbonyl]amino]-6-methyl-4,9-dioxo-1,5- |
| | | dioxonan-7-yl] 2-methylpropanoate |
| D-302 | I.Ka-5 | (3S,6S,7R,8R)-3-[[(3-hydroxy-4-methoxy-2- |
| | | pyridinyl)carbonyl]amino]-6 methyl-4,9-dioxo-8-(phenylmethyl)-1,5- |
| | | dioxonan-7-yl 2-methylpropanoate |
| D-303 | I.Ka-5 | Fluxapyroxad |
| D-304 | I.Ka-5 | Boscalid |
| D-305 | I.Ka-5 | Bixafen |
| D-306 | I.Ka-5 | Isopyrazam |
| D-307 | I.Ka-5 | Benzovindiflupyr |
| D-308 | I.Ka-5 | Fluopyram |
| D-309 | I.Ka-5 | N-[(5-chloro-2-isopropyl-phenyl)methyl]-N-cyclopropyl-5-fluoro-1,3- |
| | | dimethyl-pyrazole-4-carboxamide |
| D-310 | I.Ka-5 | Sedaxane |
| D-311 | I.Ka-5 | Penflufen |
| D-312 | I.Ka-5 | N-[2-(2,4-dichlorophenyl)-2-methoxy-1-methyl-ethyl]-3- |
| | | (difluoromethyl)-1-methyl-pyrazole-4-carboxamide |
| D-313 | I.Ka-5 | 3 (difluoromethyl)-1-methyl-N-(1,1,3-trimethylindan-4-yl)pyrazole-4- |
| | | carboxamide |
| D-314 | I.Ka-5 | 3 (trifluoromethyl)-1-methyl-N-(1,1,3-trimethylindan-4-yl)pyrazole-4- |
| | | carboxamide |
| D-315 | I.Ka-5 | 1,3-dimethyl-N-(1,1,3-trimethylindan-4-yl)pyrazole-4-carboxamide |
| D-316 | I.Ka-5 | 3-(trifluorometh¬yl)-1,5-dimethyl-N-(1,1,3-trimethylindan-4- |
| | | yl)pyrazole-4-carboxamide |
| D-317 | I.Ka-5 | 1,3,5-trimethyl-N-(1,1,3-trimethylindan-4-yl)pyrazole-4-carboxamide |
| D-318 | I.Ka-5 | 1-[3-chloro-2-[[[1-(4-chlorophenyl)-1H-pyrazol-3- |
| | | yl]oxy]methyl]phenyl]-1,4-dihydro-4-methyl-5H-tetrazol-5-one |
| D-319 | I.Ka-5 | Ametoctradin |
| D-320 | I.Ka-5 | epoxiconazole |
| D-321 | I.Ka-5 | metconazole |
| D-322 | I.Ka-5 | prothioconazole |
| D-323 | I.Ka-5 | difenoconazole |
| D-324 | I.Ka-5 | fluquinconazole |
| D-325 | I.Ka-5 | propiconazole |
| D-326 | I.Ka-5 | tebuconazole |
| D-327 | I.Ka-5 | 2-[2-chloro-4-(4-chlorophenoxy)phenyl]-1 (1,2,4-triazol-1-yl)pentan- |
| | | 2-ol |
| D-328 | I.Ka-5 | 1-[4-(4-chlorophenoxy)-2-(trifluoromethyl)phenyl]-1 cyclopropyl-2- |
| | | (1,2,4-triazol-1-yl)ethanol |
| D-329 | I.Ka-5 | 2-[4-(4-chlorophenoxy)-2-(trifluoromethyl)phenyl]-1-(1,2,4-triazol-1- |
| | | yl)butan-2-ol |
| D-330 | I.Ka-5 | 2-[2-chloro-4-(4-chlorophenoxy)phenyl]-1-(1,2,4-triazol-1-yl)butan- |
| | | 2-ol |
| D-331 | I.Ka-5 | 2-[4-(4-chlorophenoxy)-2-(trifluoromethyl)phenyl]-3-methyl-1-(1,2,4- |
| | | triazol-1-yl)butan-2-ol |
| D-332 | I.Ka-5 | 2-[4-(4-chlorophenoxy)-2-(trifluoromethyl)phenyl]-1-(1,2,4-triazol-1- |
| | | yl)propan-2-ol |
| D-333 | I.Ka-5 | 2-[2-chloro-4-(4-chlorophenoxy)phenyl]-3-methyl-1-(1,2,4-triazol-1- |
| | | yl)butan-2-ol |
| D-334 | I.Ka-5 | 2-[4-(4-chlorophenoxy)-2-(trifluoromethyl)phenyl]-1-(1,2,4-triazol-1- |
| | | yl)pentan-2-ol |
| D-335 | I.Ka-5 | 2-[4-(4-fluorophenoxy)-2-(trifluoromethyl)phenyl]-1-(1,2,4-triazol-1- |
| | | yl)propan-2-ol |
| D-336 | I.Ka-5 | 2-[2-chloro-4-(4-chlorophenoxy)phenyl]-1-(1,2,4-triazol-1-yl)pent-3- |
| | | yn-2-ol |
| D-337 | I.Ka-5 | prochloraz |
| D-338 | I.Ka-5 | [3-(4-chloro-2-fluoro-phenyl)-5-(2,4-difluorophenyl)isoxazol-4-yl]-(3- |
| | | pyridyl)methanol |
| D-339 | I.Ka-5 | fenpropimorph |
| D-340 | I.Ka-5 | Metalaxyl |
| D-341 | I.Ka-5 | Benalaxyl |
| D-342 | I.Ka-5 | Thiophanate-methyl |
| D-343 | I.Ka-5 | Carbendazim |
| D-344 | I.Ka-5 | Metrafenone |
| D-345 | I.Ka-5 | Pyrimethanil |
| D-346 | I.Ka-5 | Iprodione |
| D-347 | I.Ka-5 | Vinclozolin |
| D-348 | I.Ka-5 | Fludioxonil |
| D-349 | I.Ka-5 | dimethomorph |
| D-350 | I.Ka-5 | oxathiapiprolin |
| D-351 | I.Ka-5 | metiram |
| D-352 | I.Ka-5 | mancozeb |
| D-353 | I.Ka-5 | chlorothalonil |
| D-354 | I.Ka-5 | dithianon |
| D-355 | I.Ka-5 | Dipymetitrone |
| D-356 | I.Ka-5 | prohexadione-calcium |
| D-357 | I.Ka-5 | 3-(5-fluoro-3,3,4,4-tetramethyl-3,4-dihydroisoquinolin-1-yl)quinoline |
| D-358 | I.Ka-5 | 3-(4,4-difluoro-3,3-dimethyl-3,4-dihydroisoquinolin-1-yl)quinoline |
| D-359 | I.Ka-5 | 3-(4,4,5-trifluoro-3,3-dimethyl-3,4-dihydroisoquinolin-1-yl)quinoline |
| D-360 | I.Ka-5 | 9-fluoro-2,2-dimethyl-5-(3-quinolyl)-3H 1,4-benzoxazepine |
| D-361 | I.Ka-6 | Pyraclostrobin |
| D-362 | I.Ka-6 | Azoxystrobin |
| D-363 | I.Ka-6 | Trifloxystrobin |
| D-364 | I.Ka-6 | Picoxystrobin |
| D-365 | I.Ka-6 | Fluoxastrobin |
| D-366 | I.Ka-6 | Dimoxystrobin |
| D-367 | I.Ka-6 | Kresoxim-methyl |
| D-368 | I.Ka-6 | (2E,3Z)-5-[[1-(2,4-dichlorophenyl)-1H-pyrazol-3-yl]oxy]-2- |
| | | (methoxyimino)-N,3-dimethyl-pent-3-enamide |
| D-369 | I.Ka-6 | (2E,3Z)-5-[[1-(4-chlorophenyl)-1H-pyrazol-3-yl]oxy]-2- |
| | | (methoxyimino)-N,3-dimethyl-pent-3-enamide |
| D-370 | I.Ka-6 | [(3S,6S,7R,8R)-8-benzyl-3-[(3-acetoxy-4 methoxy-pyridine-2- |
| | | carbonyl)amino]-6-methyl-4,9-dioxo-1,5-dioxonan-7-yl] 2 |
| | | methylpropanoate |
| D-371 | I.Ka-6 | [(3S,6S,7R,8R)-8-benzyl-3-[[3-(acetoxymethoxy)-4-methoxy- |
| | | pyridine-2 carbonyl]amino]-6-methyl-4,9-dioxo-1,5-dioxonan-7-yl] 2 |
| | | methylpropanoate |
| D-372 | I.Ka-6 | [(3S,6S,7R,8R)-8-benzyl-3-[(3-isobutoxycarbonyloxy-4-methoxy- |
| | | pyridine-2 carbonyl)amino]-6-methyl-4,9-dioxo-1,5-dioxonan-7-yl] 2- |
| | | methylpropanoate |
| D-373 | I.Ka-6 | [(3S,6S,7R,8R)-8-benzyl-3-[[3-(1,3-benzodioxol-5-ylmethoxy)-4- |
| | | methoxy-pyridine-2-carbonyl]amino]-6-methyl-4,9-dioxo-1,5- |
| | | dioxonan-7-yl] 2-methylpropanoate |
| D-374 | I.Ka-6 | (3S,6S,7R,8R)-3-[[(3-hydroxy-4-methoxy-2- |
| | | pyridinyl)carbonyl]amino]-6 methyl-4,9-dioxo-8-(phenylmethyl)-1,5- |
| | | dioxonan-7-yl 2-methylpropanoate |
| D-375 | I.Ka-6 | Fluxapyroxad |
| D-376 | I.Ka-6 | Boscalid |
| D-377 | I.Ka-6 | Bixafen |
| D-378 | I.Ka-6 | Isopyrazam |
| D-379 | I.Ka-6 | Benzovindiflupyr |
| D-380 | I.Ka-6 | Fluopyram |
| D-381 | I.Ka-6 | N-[(5-chloro-2-isopropyl-phenyl)methyl]-N-cyclopropyl-5-fluoro-1,3- |
| | | dimethyl-pyrazole-4-carboxamide |
| D-382 | I.Ka-6 | Sedaxane |
| D-383 | I.Ka-6 | Penflufen |
| D-384 | I.Ka-6 | N-[2-(2,4-dichlorophenyl)-2-methoxy-1-methyl-ethyl]-3- |
| | | (difluoromethyl)-1-methyl-pyrazole-4-carboxamide |
| D-385 | I.Ka-6 | 3 (difluoromethyl)-1-methyl-N-(1,1,3-trimethylindan-4-yl)pyrazole-4- |
| | | carboxamide |
| D-386 | I.Ka-6 | 3 (trifluoromethyl)-1-methyl-N-(1,1,3-trimethylindan-4-yl)pyrazole-4- |
| | | carboxamide |
| D-387 | I.Ka-6 | 1,3-dimethyl-N-(1,1,3-trimethylindan-4-yl)pyrazole-4-carboxamide |
| D-388 | I.Ka-6 | 3-(trifluorometh¬yl)-1,5-dimethyl-N-(1,1,3-trimethylindan-4- |
| | | yl)pyrazole-4-carboxamide |
| D-389 | I.Ka-6 | 1,3,5-trimethyl-N-(1,1,3-trimethylindan-4-yl)pyrazole-4-carboxamide |
| D-390 | I.Ka-6 | 1-[3-chloro-2-[[[1-(4-chlorophenyl)-1H-pyrazol-3- |
| | | yl]oxy]methyl]phenyl]-1,4-dihydro-4-methyl-5H-tetrazol-5-one |
| D-391 | I.Ka-6 | Ametoctradin |
| D-392 | I.Ka-6 | epoxiconazole |
| D-393 | I.Ka-6 | metconazole |
| D-394 | I.Ka-6 | prothioconazole |
| D-395 | I.Ka-6 | difenoconazole |
| D-396 | I.Ka-6 | fluquinconazole |
| D-397 | I.Ka-6 | propiconazole |
| D-398 | I.Ka-6 | tebuconazole |
| D-399 | I.Ka-6 | 2-[2-chloro-4-(4-chlorophenoxy)phenyl]-1 (1,2,4-triazol-1-yl)pentan- |
| | | 2-ol |
| D-400 | I.Ka-6 | 1-[4-(4-chlorophenoxy)-2-(trifluoromethyl)phenyl]-1 cyclopropyl-2- |
| | | (1,2,4-triazol-1-yl)ethanol |
| D-401 | I.Ka-6 | 2-[4-(4-chlorophenoxy)-2-(trifluoromethyl)phenyl]-1-(1,2,4-triazol-1- |
| | | yl)butan-2-ol |
| D-402 | I.Ka-6 | 2-[2-chloro-4-(4-chlorophenoxy)phenyl]-1-(1,2,4-triazol-1-yl)butan- |
| | | 2-ol |
| D-403 | I.Ka-6 | 2-[4-(4-chlorophenoxy)-2-(trifluoromethyl)phenyl]-3-methyl-1-(1,2,4- |
| | | triazol-1-yl)butan-2-ol |
| D-404 | I.Ka-6 | 2-[4-(4-chlorophenoxy)-2-(trifluoromethyl)phenyl]-1-(1,2,4-triazol-1- |
| | | yl)propan-2-ol |
| D-405 | I.Ka-6 | 2-[2-chloro-4-(4-chlorophenoxy)phenyl]-3-methyl-1-(1,2,4-triazol-1- |
| | | yl)butan-2-ol |
| D-406 | I.Ka-6 | 2-[4-(4-chlorophenoxy)-2-(trifluoromethyl)phenyl]-1-(1,2,4-triazol-1- |
| | | yl)pentan-2-ol |
| D-407 | I.Ka-6 | 2-[4-(4-fluorophenoxy)-2-(trifluoromethyl)phenyl]-1-(1,2,4-triazol-1- |
| | | yl)propan-2-ol |
| D-408 | I.Ka-6 | 2-[2-chloro-4-(4-chlorophenoxy)phenyl]-1-(1,2,4-triazol-1-yl)pent-3- |
| | | yn-2-ol |
| D-409 | I.Ka-6 | prochloraz |
| D-410 | I.Ka-6 | [3-(4-chloro-2-fluoro-phenyl)-5-(2,4-difluorophenyl)isoxazol-4-yl]-(3- |
| | | pyridyl)methanol |
| D-411 | I.Ka-6 | fenpropimorph |
| D-412 | I.Ka-6 | Metalaxyl |
| D-413 | I.Ka-6 | Benalaxyl |
| D-414 | I.Ka-6 | Thiophanate-methyl |
| D-415 | I.Ka-6 | Carbendazim |
| D-416 | I.Ka-6 | Metrafenone |
| D-417 | I.Ka-6 | Pyrimethanil |
| D-418 | I.Ka-6 | Iprodione |
| D-419 | I.Ka-6 | Vinclozolin |
| D-420 | I.Ka-6 | Fludioxonil |
| D-421 | I.Ka-6 | dimethomorph |
| D-422 | I.Ka-6 | oxathiapiprolin |
| D-423 | I.Ka-6 | metiram |
| D-424 | I.Ka-6 | mancozeb |
| D-425 | I.Ka-6 | chlorothalonil |
| D-426 | I.Ka-6 | dithianon |
| D-427 | I.Ka-6 | Dipymetitrone |
| D-428 | I.Ka-6 | prohexadione-calcium |
| D-429 | I.Ka-6 | 3-(5-fluoro-3,3,4,4-tetramethyl-3,4-dihydroisoquinolin-1-yl)quinoline |
| D-430 | I.Ka-6 | 3-(4,4-difluoro-3,3-dimethyl-3,4-dihydroisoquinolin-1-yl)quinoline |
| D-431 | I.Ka-6 | 3-(4,4,5-trifluoro-3,3-dimethyl-3,4-dihydroisoquinolin-1-yl)quinoline |
| D-432 | I.Ka-6 | 9-fluoro-2,2-dimethyl-5-(3-quinolyl)-3H 1,4-benzoxazepine |
| D-433 | I.Ka-7 | Pyraclostrobin |
| D-434 | I.Ka-7 | Azoxystrobin |
| D-435 | I.Ka-7 | Trifloxystrobin |
| D-436 | I.Ka-7 | Picoxystrobin |
| D-437 | I.Ka-7 | Fluoxastrobin |
| D-438 | I.Ka-7 | Dimoxystrobin |
| D-439 | I.Ka-7 | Kresoxim-methyl |
| D-440 | I.Ka-7 | (2E,3Z)-5-[[1-(2,4-dichlorophenyl)-1H-pyrazol-3-yl]oxy]-2- |
| | | (methoxyimino)-N,3-dimethyl-pent-3-enamide |
| D-441 | I.Ka-7 | (2E,3Z)-5-[[1-(4-chlorophenyl)-1H-pyrazol-3-yl]oxy]-2- |
| | | (methoxyimino)-N,3-dimethyl-pent-3-enamide |
| D-442 | I.Ka-7 | [(3S,6S,7R,8R)-8-benzyl-3-[(3-acetoxy-4 methoxy-pyridine-2- |
| | | carbonyl)amino]-6-methyl-4,9-dioxo-1,5-dioxonan-7-yl] 2 |
| | | methylpropanoate |
| D-443 | I.Ka-7 | [(3S,6S,7R,8R)-8-benzyl-3-[[3-(acetoxymethoxy)-4-methoxy- |
| | | pyridine-2 carbonyl]amino]-6-methyl-4,9-dioxo-1,5-dioxonan-7-yl] 2 |
| | | methylpropanoate |
| D-444 | I.Ka-7 | [(3S,6S,7R,8R)-8-benzyl-3-[(3-isobutoxycarbonyloxy-4-methoxy- |
| | | pyridine-2 carbonyl)amino]-6-methyl-4,9-dioxo-1,5-dioxonan-7-yl] 2- |
| | | methylpropanoate |
| D-445 | I.Ka-7 | [(3S,6S,7R,8R)-8-benzyl-3-[[3-(1,3-benzodioxol-5-ylmethoxy)-4- |
| | | methoxy-pyridine-2-carbonyl]amino]-6-methyl-4,9-dioxo-1,5- |
| | | dioxonan-7-yl] 2-methylpropanoate |
| D-446 | I.Ka-7 | (3S,6S,7R,8R)-3-[[(3-hydroxy-4-methoxy-2- |
| | | pyridinyl)carbonyl]amino]-6 methyl-4,9-dioxo-8-(phenylmethyl)-1,5- |
| | | dioxonan-7-yl 2-methylpropanoate |
| D-447 | I.Ka-7 | Fluxapyroxad |
| D-448 | I.Ka-7 | Boscalid |
| D-449 | I.Ka-7 | Bixafen |
| D-450 | I.Ka-7 | Isopyrazam |
| D-451 | I.Ka-7 | Benzovindiflupyr |
| D-452 | I.Ka-7 | Fluopyram |
| D-453 | I.Ka-7 | N-[(5-chloro-2-isopropyl-phenyl)methyl]-N-cyclopropyl-5-fluoro-1,3- |
| | | dimethyl-pyrazole-4-carboxamide |
| D-454 | I.Ka-7 | Sedaxane |
| D-455 | I.Ka-7 | Penflufen |
| D-456 | I.Ka-7 | N-[2-(2,4-dichlorophenyl)-2-methoxy-1-methyl-ethyl]-3- |
| | | (difluoromethyl)-1-methyl-pyrazole-4-carboxamide |
| D-457 | I.Ka-7 | 3 (difluoromethyl)-1-methyl-N-(1,1,3-trimethylindan-4-yl)pyrazole-4- |
| | | carboxamide |
| D-458 | I.Ka-7 | 3 (trifluoromethyl)-1-methyl-N-(1,1,3-trimethylindan-4-yl)pyrazole-4- |
| | | carboxamide |
| D-459 | I.Ka-7 | 1,3-dimethyl-N-(1,1,3-trimethylindan-4-yl)pyrazole-4-carboxamide |
| D-460 | I.Ka-7 | 3-(trifluorometh¬yl)-1,5-dimethyl-N-(1,1,3-trimethylindan-4- |
| | | yl)pyrazole-4-carboxamide |
| D-461 | I.Ka-7 | 1,3,5-trimethyl-N-(1,1,3-trimethylindan-4-yl)pyrazole-4-carboxamide |
| D-462 | I.Ka-7 | 1-[3-chloro-2-[[[1-(4-chlorophenyl)-1H-pyrazol-3- |
| | | yl]oxy]methyl]phenyl]-1,4-dihydro-4-methyl-5H-tetrazol-5-one |
| D-463 | I.Ka-7 | Ametoctradin |
| D-464 | I.Ka-7 | epoxiconazole |
| D-465 | I.Ka-7 | metconazole |
| D-466 | I.Ka-7 | prothioconazole |
| D-467 | I.Ka-7 | difenoconazole |
| D-468 | I.Ka-7 | fluquinconazole |
| D-469 | I.Ka-7 | propiconazole |
| D-470 | I.Ka-7 | tebuconazole |
| D-471 | I.Ka-7 | 2-[2-chloro-4-(4-chlorophenoxy)phenyl]-1 (1,2,4-triazol-1-yl)pentan- |
| | | 2-ol |
| D-472 | I.Ka-7 | 1-[4-(4-chlorophenoxy)-2-(trifluoromethyl)phenyl]-1 cyclopropyl-2- |
| | | (1,2,4-triazol-1-yl)ethanol |
| D-473 | I.Ka-7 | 2-[4-(4-chlorophenoxy)-2-(trifluoromethyl)phenyl]-1-(1,2,4-triazol-1- |
| | | yl)butan-2-ol |
| D-474 | I.Ka-7 | 2-[2-chloro-4-(4-chlorophenoxy)phenyl]-1-(1,2,4-triazol-1-yl)butan- |
| | | 2-ol |
| D-475 | I.Ka-7 | 2-[4-(4-chlorophenoxy)-2-(trifluoromethyl)phenyl]-3-methyl-1-(1,2,4- |
| | | triazol-1-yl)butan-2-ol |
| D-476 | I.Ka-7 | 2-[4-(4-chlorophenoxy)-2-(trifluoromethyl)phenyl]-1-(1,2,4-triazol-1- |
| | | yl)propan-2-ol |
| D-477 | I.Ka-7 | 2-[2-chloro-4-(4-chlorophenoxy)phenyl]-3-methyl-1-(1,2,4-triazol-1- |
| | | yl)butan-2-ol |
| D-478 | I.Ka-7 | 2-[4-(4-chlorophenoxy)-2-(trifluoromethyl)phenyl]-1-(1,2,4-triazol-1- |
| | | yl)pentan-2-ol |
| D-479 | I.Ka-7 | 2-[4-(4-fluorophenoxy)-2-(trifluoromethyl)phenyl]-1-(1,2,4-triazol-1- |
| | | yl)propan-2-ol |
| D-480 | I.Ka-7 | 2-[2-chloro-4-(4-chlorophenoxy)phenyl]-1-(1,2,4-triazol-1-yl)pent-3- |
| | | yn-2-ol |
| D-481 | I.Ka-7 | prochloraz |
| D-482 | I.Ka-7 | [3-(4-chloro-2-fluoro-phenyl)-5-(2,4-difluorophenyl)isoxazol-4-yl]-(3- |
| | | pyridyl)methanol |
| D-483 | I.Ka-7 | fenpropimorph |
| D-484 | I.Ka-7 | Metalaxyl |
| D-485 | I.Ka-7 | Benalaxyl |
| D-486 | I.Ka-7 | Thiophanate-methyl |
| D-487 | I.Ka-7 | Carbendazim |
| D-488 | I.Ka-7 | Metrafenone |
| D-489 | I.Ka-7 | Pyrimethanil |
| D-490 | I.Ka-7 | Iprodione |
| D-491 | I.Ka-7 | Vinclozolin |
| D-492 | I.Ka-7 | Fludioxonil |
| D-493 | I.Ka-7 | dimethomorph |
| D-494 | I.Ka-7 | oxathiapiprolin |
| D-495 | I.Ka-7 | metiram |
| D-496 | I.Ka-7 | mancozeb |
| D-497 | I.Ka-7 | chlorothalonil |
| D-498 | I.Ka-7 | dithianon |
| D-499 | I.Ka-7 | Dipymetitrone |
| D-500 | I.Ka-7 | prohexadione-calcium |
| D-501 | I.Ka-7 | 3-(5-fluoro-3,3,4,4-tetramethyl-3,4-dihydroisoquinolin-1-yl)quinoline |
| D-502 | I.Ka-7 | 3-(4,4-difluoro-3,3-dimethyl-3,4-dihydroisoquinolin-1-yl)quinoline |
| D-503 | I.Ka-7 | 3-(4,4,5-trifluoro-3,3-dimethyl-3,4-dihydroisoquinolin-1-yl)quinoline |
| D-504 | I.Ka-7 | 9-fluoro-2,2-dimethyl-5-(3-quinolyl)-3H 1,4-benzoxazepine |
| D-505 | I.Ka-8 | Pyraclostrobin |
| D-506 | I.Ka-8 | Azoxystrobin |
| D-507 | I.Ka-8 | Trifloxystrobin |
| D-508 | I.Ka-8 | Picoxystrobin |
| D-509 | I.Ka-8 | Fluoxastrobin |
| D-510 | I.Ka-8 | Dimoxystrobin |
| D-511 | I.Ka-8 | Kresoxim-methyl |
| D-512 | I.Ka-8 | (2E,3Z)-5-[[1-(2,4-dichlorophenyl)-1H-pyrazol-3-yl]oxy]-2- |
| | | (methoxyimino)-N,3-dimethyl-pent-3-enamide |
| D-513 | I.Ka-8 | (2E,3Z)-5-[[1-(4-chlorophenyl)-1H-pyrazol-3-yl]oxy]-2- |
| | | (methoxyimino)-N,3-dimethyl-pent-3-enamide |
| D-514 | I.Ka-8 | [(3S,6S,7R,8R)-8-benzyl-3-[(3-acetoxy-4 methoxy-pyridine-2- |
| | | carbonyl)amino]-6-methyl-4,9-dioxo-1,5-dioxonan-7-yl] 2 |
| | | methylpropanoate |
| D-515 | I.Ka-8 | [(3S,6S,7R,8R)-8-benzyl-3-[[3-(acetoxymethoxy)-4-methoxy- |
| | | pyridine-2 carbonyl]amino]-6-methyl-4,9-dioxo-1,5-dioxonan-7-yl] 2 |
| | | methylpropanoate |
| D-516 | I.Ka-8 | [(3S,6S,7R,8R)-8-benzyl-3-[(3-isobutoxycarbonyloxy-4-methoxy- |
| | | pyridine-2 carbonyl)amino]-6-methyl-4,9-dioxo-1,5-dioxonan-7-yl] 2- |
| | | methylpropanoate |
| D-517 | I.Ka-8 | [(3S,6S,7R,8R)-8-benzyl-3-[[3-(1,3-benzodioxol-5-ylmethoxy)-4- |
| | | methoxy-pyridine-2-carbonyl]amino]-6-methyl-4,9-dioxo-1,5- |
| | | dioxonan-7-yl] 2-methylpropanoate |
| D-518 | I.Ka-8 | (3S,6S,7R,8R)-3-[[(3-hydroxy-4-methoxy-2- |
| | | pyridinyl)carbonyl]amino]-6 methyl-4,9-dioxo-8-(phenylmethyl)-1,5- |
| | | dioxonan-7-yl 2-methylpropanoate |
| D-519 | I.Ka-8 | Fluxapyroxad |
| D-520 | I.Ka-8 | Boscalid |
| D-521 | I.Ka-8 | Bixafen |
| D-522 | I.Ka-8 | Isopyrazam |
| D-523 | I.Ka-8 | Benzovindiflupyr |
| D-524 | I.Ka-8 | Fluopyram |
| D-525 | I.Ka-8 | N-[(5-chloro-2-isopropyl-phenyl)methyl]-N-cyclopropyl-5-fluoro-1,3- |
| | | dimethyl-pyrazole-4-carboxamide |
| D-526 | I.Ka-8 | Sedaxane |
| D-527 | I.Ka-8 | Penflufen |
| D-528 | I.Ka-8 | N-[2-(2,4-dichlorophenyl)-2-methoxy-1-methyl-ethyl]-3- |
| | | (difluoromethyl)-1-methyl-pyrazole-4-carboxamide |
| D-529 | I.Ka-8 | 3 (difluoromethyl)-1-methyl-N-(1,1,3-trimethylindan-4-yl)pyrazole-4- |
| | | carboxamide |
| D-530 | I.Ka-8 | 3 (trifluoromethyl)-1-methyl-N-(1,1,3-trimethylindan-4-yl)pyrazole-4- |
| | | carboxamide |
| D-531 | I.Ka-8 | 1,3-dimethyl-N-(1,1,3-trimethylindan-4-yl)pyrazole-4-carboxamide |
| D-532 | I.Ka-8 | 3-(trifluorometh¬yl)-1,5-dimethyl-N-(1,1,3-trimethylindan-4- |
| | | yl)pyrazole-4-carboxamide |
| D-533 | I.Ka-8 | 1,3,5-trimethyl-N-(1,1,3-trimethylindan-4-yl)pyrazole-4-carboxamide |
| D-534 | I.Ka-8 | 1-[3-chloro-2-[[[1-(4-chlorophenyl)-1H-pyrazol-3- |
| | | yl]oxy]methyl]phenyl]-1,4-dihydro-4-methyl-5H-tetrazol-5-one |
| D-535 | I.Ka-8 | Ametoctradin |
| D-536 | I.Ka-8 | epoxiconazole |
| D-537 | I.Ka-8 | metconazole |
| D-538 | I.Ka-8 | prothioconazole |
| D-539 | I.Ka-8 | difenoconazole |
| D-540 | I.Ka-8 | fluquinconazole |
| D-541 | I.Ka-8 | propiconazole |
| D-542 | I.Ka-8 | tebuconazole |
| D-543 | I.Ka-8 | 2-[2-chloro-4-(4-chlorophenoxy)phenyl]-1 (1,2,4-triazol-1-yl)pentan- |
| | | 2-ol |
| D-544 | I.Ka-8 | 1-[4-(4-chlorophenoxy)-2-(trifluoromethyl)phenyl]-1 cyclopropyl-2- |
| | | (1,2,4-triazol-1-yl)ethanol |
| D-545 | I.Ka-8 | 2-[4-(4-chlorophenoxy)-2-(trifluoromethyl)phenyl]-1-(1,2,4-triazol-1- |
| | | yl)butan-2-ol |
| D-546 | I.Ka-8 | 2-[2-chloro-4-(4-chlorophenoxy)phenyl]-1-(1,2,4-triazol-1-yl)butan- |
| | | 2-ol |
| D-547 | I.Ka-8 | 2-[4-(4-chlorophenoxy)-2-(trifluoromethyl)phenyl]-3-methyl-1-(1,2,4- |
| | | triazol-1-yl)butan-2-ol |
| D-548 | I.Ka-8 | 2-[4-(4-chlorophenoxy)-2-(trifluoromethyl)phenyl]-1-(1,2,4-triazol-1- |
| | | yl)propan-2-ol |
| D-549 | I.Ka-8 | 2-[2-chloro-4-(4-chlorophenoxy)phenyl]-3-methyl-1-(1,2,4-triazol-1- |
| | | yl)butan-2-ol |
| D-550 | I.Ka-8 | 2-[4-(4-chlorophenoxy)-2-(trifluoromethyl)phenyl]-1-(1,2,4-triazol-1- |
| | | yl)pentan-2-ol |
| D-551 | I.Ka-8 | 2-[4-(4-fluorophenoxy)-2-(trifluoromethyl)phenyl]-1-(1,2,4-triazol-1- |
| | | yl)propan-2-ol |
| D-552 | I.Ka-8 | 2-[2-chloro-4-(4-chlorophenoxy)phenyl]-1-(1,2,4-triazol-1-yl)pent-3- |
| | | yn-2-ol |
| D-553 | I.Ka-8 | prochloraz |
| D-554 | I.Ka-8 | [3-(4-chloro-2-fluoro-phenyl)-5-(2,4-difluorophenyl)isoxazol-4-yl]-(3- |
| | | pyridyl)methanol |
| D-555 | I.Ka-8 | fenpropimorph |
| D-556 | I.Ka-8 | Metalaxyl |
| D-557 | I.Ka-8 | Benalaxyl |
| D-558 | I.Ka-8 | Thiophanate-methyl |
| D-559 | I.Ka-8 | Carbendazim |
| D-560 | I.Ka-8 | Metrafenone |
| D-561 | I.Ka-8 | Pyrimethanil |
| D-562 | I.Ka-8 | Iprodione |
| D-563 | I.Ka-8 | Vinclozolin |
| D-564 | I.Ka-8 | Fludioxonil |
| D-565 | I.Ka-8 | dimethomorph |
| D-566 | I.Ka-8 | oxathiapiprolin |
| D-567 | I.Ka-8 | metiram |
| D-568 | I.Ka-8 | mancozeb |
| D-569 | I.Ka-8 | chlorothalonil |
| D-570 | I.Ka-8 | dithianon |
| D-571 | I.Ka-8 | Dipymetitrone |
| D-572 | I.Ka-8 | prohexadione-calcium |
| D-573 | I.Ka-8 | 3-(5-fluoro-3,3,4,4-tetramethyl-3,4-dihydroisoquinolin-1-yl)quinoline |
| D-574 | I.Ka-8 | 3-(4,4-difluoro-3,3-dimethyl-3,4-dihydroisoquinolin-1-yl)quinoline |
| D-575 | I.Ka-8 | 3-(4,4,5-trifluoro-3,3-dimethyl-3,4-dihydroisoquinolin-1-yl)quinoline |
| D-576 | I.Ka-8 | 9-fluoro-2,2-dimethyl-5-(3-quinolyl)-3H 1,4-benzoxazepine |
| D-577 | I.Ka-9 | Pyraclostrobin |
| D-578 | I.Ka-9 | Azoxystrobin |
| D-579 | I.Ka-9 | Trifloxystrobin |
| D-580 | I.Ka-9 | Picoxystrobin |
| D-581 | I.Ka-9 | Fluoxastrobin |
| D-582 | I.Ka-9 | Dimoxystrobin |
| D-583 | I.Ka-9 | Kresoxim-methyl |
| D-584 | I.Ka-9 | (2E,3Z)-5-[[1-(2,4-dichlorophenyl)-1H-pyrazol-3-yl]oxy]-2- |
| | | (methoxyimino)-N,3-dimethyl-pent-3-enamide |
| D-585 | I.Ka-9 | (2E,3Z)-5-[[1-(4-chlorophenyl)-1H-pyrazol-3-yl]oxy]-2- |
| | | (methoxyimino)-N,3-dimethyl-pent-3-enamide |
| D-586 | I.Ka-9 | [(3S,6S,7R,8R)-8-benzyl-3-[(3-acetoxy-4 methoxy-pyridine-2- |
| | | carbonyl)amino]-6-methyl-4,9-dioxo-1,5-dioxonan-7-yl] 2 |
| | | methylpropanoate |
| D-587 | I.Ka-9 | [(3S,6S,7R,8R)-8-benzyl-3-[[3-(acetoxymethoxy)-4-methoxy- |
| | | pyridine-2 carbonyl]amino]-6-methyl-4,9-dioxo-1,5-dioxonan-7-yl] 2 |
| | | methylpropanoate |
| D-588 | I.Ka-9 | [(3S,6S,7R,8R)-8-benzyl-3-[(3-isobutoxycarbonyloxy-4-methoxy- |
| | | pyridine-2 carbonyl)amino]-6-methyl-4,9-dioxo-1,5-dioxonan-7-yl] 2- |
| | | methylpropanoate |
| D-589 | I.Ka-9 | [(3S,6S,7R,8R)-8-benzyl-3-[[3-(1,3-benzodioxol-5-ylmethoxy)-4- |
| | | methoxy-pyridine-2-carbonyl]amino]-6-methyl-4,9-dioxo-1,5- |
| | | dioxonan-7-yl] 2-methylpropanoate |
| D-590 | I.Ka-9 | (3S,6S,7R,8R)-3-[[(3-hydroxy-4-methoxy-2- |
| | | pyridinyl)carbonyl]amino]-6 methyl-4,9-dioxo-8-(phenylmethyl)-1,5- |
| | | dioxonan-7-yl 2-methylpropanoate |
| D-591 | I.Ka-9 | Fluxapyroxad |
| D-592 | I.Ka-9 | Boscalid |
| D-593 | I.Ka-9 | Bixafen |
| D-594 | I.Ka-9 | Isopyrazam |
| D-595 | I.Ka-9 | Benzovindiflupyr |
| D-596 | I.Ka-9 | Fluopyram |
| D-597 | I.Ka-9 | N-[(5-chloro-2-isopropyl-phenyl)methyl]-N-cyclopropyl-5-fluoro-1,3- |
| | | dimethyl-pyrazole-4-carboxamide |
| D-598 | I.Ka-9 | Sedaxane |
| D-599 | I.Ka-9 | Penflufen |
| D-600 | I.Ka-9 | N-[2-(2,4-dichlorophenyl)-2-methoxy-1-methyl-ethyl]-3- |
| | | (difluoromethyl)-1-methyl-pyrazole-4-carboxamide |
| D-601 | I.Ka-9 | 3 (difluoromethyl)-1-methyl-N-(1,1,3-trimethylindan-4-yl)pyrazole-4- |
| | | carboxamide |
| D-602 | I.Ka-9 | 3 (trifluoromethyl)-1-methyl-N-(1,1,3-trimethylindan-4-yl)pyrazole-4- |
| | | carboxamide |
| D-603 | I.Ka-9 | 1,3-dimethyl-N-(1,1,3-trimethylindan-4-yl)pyrazole-4-carboxamide |
| D-604 | I.Ka-9 | 3-(trifluorometh¬yl)-1,5-dimethyl-N-(1,1,3-trimethylindan-4- |
| | | yl)pyrazole-4-carboxamide |
| D-605 | I.Ka-9 | 1,3,5-trimethyl-N-(1,1,3-trimethylindan-4-yl)pyrazole-4-carboxamide |
| D-606 | I.Ka-9 | 1-[3-chloro-2-[[[1-(4-chlorophenyl)-1H-pyrazol-3- |
| | | yl]oxy]methyl]phenyl]-1,4-dihydro-4-methyl-5H-tetrazol-5-one |
| D-607 | I.Ka-9 | Ametoctradin |
| D-608 | I.Ka-9 | epoxiconazole |
| D-609 | I.Ka-9 | metconazole |
| D-610 | I.Ka-9 | prothioconazole |
| D-611 | I.Ka-9 | difenoconazole |
| D-612 | I.Ka-9 | fluquinconazole |
| D-613 | I.Ka-9 | propiconazole |
| D-614 | I.Ka-9 | tebuconazole |
| D-615 | I.Ka-9 | 2-[2-chloro-4-(4-chlorophenoxy)phenyl]-1 (1,2,4-triazol-1-yl)pentan- |
| | | 2-ol |
| D-616 | I.Ka-9 | 1-[4-(4-chlorophenoxy)-2-(trifluoromethyl)phenyl]-1 cyclopropyl-2- |
| | | (1,2,4-triazol-1-yl)ethanol |
| D-617 | I.Ka-9 | 2-[4-(4-chlorophenoxy)-2-(trifluoromethyl)phenyl]-1-(1,2,4-triazol-1- |
| | | yl)butan-2-ol |
| D-618 | I.Ka-9 | 2-[2-chloro-4-(4-chlorophenoxy)phenyl]-1-(1,2,4-triazol-1-yl)butan- |
| | | 2-ol |
| D-619 | I.Ka-9 | 2-[4-(4-chlorophenoxy)-2-(trifluoromethyl)phenyl]-3-methyl-1-(1,2,4- |
| | | triazol-1-yl)butan-2-ol |
| D-620 | I.Ka-9 | 2-[4-(4-chlorophenoxy)-2-(trifluoromethyl)phenyl]-1-(1,2,4-triazol-1- |
| | | yl)propan-2-ol |
| D-621 | I.Ka-9 | 2-[2-chloro-4-(4-chlorophenoxy)phenyl]-3-methyl-1-(1,2,4-triazol-1- |
| | | yl)butan-2-ol |
| D-622 | I.Ka-9 | 2-[4-(4-chlorophenoxy)-2-(trifluoromethyl)phenyl]-1-(1,2,4-triazol-1- |
| | | yl)pentan-2-ol |
| D-623 | I.Ka-9 | 2-[4-(4-fluorophenoxy)-2-(trifluoromethyl)phenyl]-1-(1,2,4-triazol-1- |
| | | yl)propan-2-ol |
| D-624 | I.Ka-9 | 2-[2-chloro-4-(4-chlorophenoxy)phenyl]-1-(1,2,4-triazol-1-yl)pent-3- |
| | | yn-2-ol |
| D-625 | I.Ka-9 | prochloraz |
| D-626 | I.Ka-9 | [3-(4-chloro-2-fluoro-phenyl)-5-(2,4-difluorophenyl)isoxazol-4-yl]-(3- |
| | | pyridyl)methanol |
| D-627 | I.Ka-9 | fenpropimorph |
| D-628 | I.Ka-9 | Metalaxyl |
| D-629 | I.Ka-9 | Benalaxyl |
| D-630 | I.Ka-9 | Thiophanate-methyl |
| D-631 | I.Ka-9 | Carbendazim |
| D-632 | I.Ka-9 | Metrafenone |
| D-633 | I.Ka-9 | Pyrimethanil |
| D-634 | I.Ka-9 | Iprodione |
| D-635 | I.Ka-9 | Vinclozolin |
| D-636 | I.Ka-9 | Fludioxonil |
| D-637 | I.Ka-9 | dimethomorph |
| D-638 | I.Ka-9 | oxathiapiprolin |
| D-639 | I.Ka-9 | metiram |
| D-640 | I.Ka-9 | mancozeb |
| D-641 | I.Ka-9 | chlorothalonil |
| D-642 | I.Ka-9 | dithianon |
| D-643 | I.Ka-9 | Dipymetitrone |
| D-644 | I.Ka-9 | prohexadione-calcium |
| D-645 | I.Ka-9 | 3-(5-fluoro-3,3,4,4-tetramethyl-3,4-dihydroisoquinolin-1-yl)quinoline |
| D-646 | I.Ka-9 | 3-(4,4-difluoro-3,3-dimethyl-3,4-dihydroisoquinolin-1-yl)quinoline |
| D-647 | I.Ka-9 | 3-(4,4,5-trifluoro-3,3-dimethyl-3,4-dihydroisoquinolin-1-yl)quinoline |
| D-648 | I.Ka-9 | 9-fluoro-2,2-dimethyl-5-(3-quinolyl)-3H 1,4-benzoxazepine |
| D-649 | I.Ka-10 | Pyraclostrobin |
| D-650 | I.Ka-10 | Azoxystrobin |
| D-651 | I.Ka-10 | Trifloxystrobin |
| D-652 | I.Ka-10 | Picoxystrobin |
| D-653 | I.Ka-10 | Fluoxastrobin |
| D-654 | I.Ka-10 | Dimoxystrobin |
| D-655 | I.Ka-10 | Kresoxim-methyl |
| D-656 | I.Ka-10 | (2E,3Z)-5-[[1-(2,4-dichlorophenyl)-1H-pyrazol-3-yl]oxy]-2- |
| | | (methoxyimino)-N,3-dimethyl-pent-3-enamide |
| D-657 | I.Ka-10 | (2E,3Z)-5-[[1-(4-chlorophenyl)-1H-pyrazol-3-yl]oxy]-2- |
| | | (methoxyimino)-N,3-dimethyl-pent-3-enamide |
| D-658 | I.Ka-10 | [(3S,6S,7R,8R)-8-benzyl-3-[(3-acetoxy-4 methoxy-pyridine-2- |
| | | carbonyl)amino]-6-methyl-4,9-dioxo-1,5-dioxonan-7-yl] 2 |
| | | methylpropanoate |
| D-659 | I.Ka-10 | [(3S,6S,7R,8R)-8-benzyl-3-[[3-(acetoxymethoxy)-4-methoxy- |
| | | pyridine-2 carbonyl]amino]-6-methyl-4,9-dioxo-1,5-dioxonan-7-yl] 2 |
| | | methylpropanoate |
| D-660 | I.Ka-10 | [(3S,6S,7R,8R)-8-benzyl-3-[(3-isobutoxycarbonyloxy-4-methoxy- |
| | | pyridine-2 carbonyl)amino]-6-methyl-4,9-dioxo-1,5-dioxonan-7-yl] 2- |
| | | methylpropanoate |
| D-661 | I.Ka-10 | [(3S,6S,7R,8R)-8-benzyl-3-[[3-(1,3-benzodioxol-5-ylmethoxy)-4- |
| | | methoxy-pyridine-2-carbonyl]amino]-6-methyl-4,9-dioxo-1,5- |
| | | dioxonan-7-yl] 2-methylpropanoate |
| D-662 | I.Ka-10 | (3S,6S,7R,8R)-3-[[(3-hydroxy-4-methoxy-2- |
| | | pyridinyl)carbonyl]amino]-6 methyl-4,9-dioxo-8-(phenylmethyl)-1,5- |
| | | dioxonan-7-yl 2-methylpropanoate |
| D-663 | I.Ka-10 | Fluxapyroxad |
| D-664 | I.Ka-10 | Boscalid |
| D-665 | I.Ka-10 | Bixafen |
| D-666 | I.Ka-10 | Isopyrazam |
| D-667 | I.Ka-10 | Benzovindiflupyr |
| D-668 | I.Ka-10 | Fluopyram |
| D-669 | I.Ka-10 | N-[(5-chloro-2-isopropyl-phenyl)methyl]-N-cyclopropyl-5-fluoro-1,3- |
| | | dimethyl-pyrazole-4-carboxamide |
| D-670 | I.Ka-10 | Sedaxane |
| D-671 | I.Ka-10 | Penflufen |
| D-672 | I.Ka-10 | N-[2-(2,4-dichlorophenyl)-2-methoxy-1-methyl-ethyl]-3- |
| | | (difluoromethyl)-1-methyl-pyrazole-4-carboxamide |
| D-673 | I.Ka-10 | 3 (difluoromethyl)-1-methyl-N-(1,1,3-trimethylindan-4-yl)pyrazole-4- |
| | | carboxamide |
| D-674 | I.Ka-10 | 3 (trifluoromethyl)-1-methyl-N-(1,1,3-trimethylindan-4-yl)pyrazole-4- |
| | | carboxamide |
| D-675 | I.Ka-10 | 1,3-dimethyl-N-(1,1,3-trimethylindan-4-yl)pyrazole-4-carboxamide |
| D-676 | I.Ka-10 | 3-(trifluorometh¬yl)-1,5-dimethyl-N-(1,1,3-trimethylindan-4- |
| | | yl)pyrazole-4-carboxamide |
| D-677 | I.Ka-10 | 1,3,5-trimethyl-N-(1,1,3-trimethylindan-4-yl)pyrazole-4-carboxamide |
| D-678 | I.Ka-10 | 1-[3-chloro-2-[[[1-(4-chlorophenyl)-1H-pyrazol-3- |
| | | yl]oxy]methyl]phenyl]-1,4-dihydro-4-methyl-5H-tetrazol-5-one |
| D-679 | I.Ka-10 | Ametoctradin |
| D-680 | I.Ka-10 | epoxiconazole |
| D-681 | I.Ka-10 | metconazole |
| D-682 | I.Ka-10 | prothioconazole |
| D-683 | I.Ka-10 | difenoconazole |
| D-684 | I.Ka-10 | fluquinconazole |
| D-685 | I.Ka-10 | propiconazole |
| D-686 | I.Ka-10 | tebuconazole |
| D-687 | I.Ka-10 | 2-[2-chloro-4-(4-chlorophenoxy)phenyl]-1 (1,2,4-triazol-1-yl)pentan- |
| | | 2-ol |
| D-688 | I.Ka-10 | 1-[4-(4-chlorophenoxy)-2-(trifluoromethyl)phenyl]-1 cyclopropyl-2- |
| | | (1,2,4-triazol-1-yl)ethanol |
| D-689 | I.Ka-10 | 2-[4-(4-chlorophenoxy)-2-(trifluoromethyl)phenyl]-1-(1,2,4-triazol-1- |
| | | yl)butan-2-ol |
| D-690 | I.Ka-10 | 2-[2-chloro-4-(4-chlorophenoxy)phenyl]-1-(1,2,4-triazol-1-yl)butan- |
| | | 2-ol |
| D-691 | I.Ka-10 | 2-[4-(4-chlorophenoxy)-2-(trifluoromethyl)phenyl]-3-methyl-1-(1,2,4- |
| | | triazol-1-yl)butan-2-ol |
| D-692 | I.Ka-10 | 2-[4-(4-chlorophenoxy)-2-(trifluoromethyl)phenyl]-1-(1,2,4-triazol-1- |
| | | yl)propan-2-ol |
| D-693 | I.Ka-10 | 2-[2-chloro-4-(4-chlorophenoxy)phenyl]-3-methyl-1-(1,2,4-triazol-1- |
| | | yl)butan-2-ol |
| D-694 | I.Ka-10 | 2-[4-(4-chlorophenoxy)-2-(trifluoromethyl)phenyl]-1-(1,2,4-triazol-1- |
| | | yl)pentan-2-ol |
| D-695 | I.Ka-10 | 2-[4-(4-fluorophenoxy)-2-(trifluoromethyl)phenyl]-1-(1,2,4-triazol-1- |
| | | yl)propan-2-ol |
| D-696 | I.Ka-10 | 2-[2-chloro-4-(4-chlorophenoxy)phenyl]-1-(1,2,4-triazol-1-yl)pent-3- |
| | | yn-2-ol |
| D-697 | I.Ka-10 | prochloraz |
| D-698 | I.Ka-10 | [3-(4-chloro-2-fluoro-phenyl)-5-(2,4-difluorophenyl)isoxazol-4-yl]-(3- |
| | | pyridyl)methanol |
| D-699 | I.Ka-10 | fenpropimorph |
| D-700 | I.Ka-10 | Metalaxyl |
| D-701 | I.Ka-10 | Benalaxyl |
| D-702 | I.Ka-10 | Thiophanate-methyl |
| D-703 | I.Ka-10 | Carbendazim |
| D-704 | I.Ka-10 | Metrafenone |
| D-705 | I.Ka-10 | Pyrimethanil |
| D-706 | I.Ka-10 | Iprodione |
| D-707 | I.Ka-10 | Vinclozolin |
| D-708 | I.Ka-10 | Fludioxonil |
| D-709 | I.Ka-10 | dimethomorph |
| D-710 | I.Ka-10 | oxathiapiprolin |
| D-711 | I.Ka-10 | metiram |
| D-712 | I.Ka-10 | mancozeb |
| D-713 | I.Ka-10 | chlorothalonil |
| D-714 | I.Ka-10 | dithianon |
| D-715 | I.Ka-10 | Dipymetitrone |
| D-716 | I.Ka-10 | prohexadione-calcium |
| D-717 | I.Ka-10 | 3-(5-fluoro-3,3,4,4-tetramethyl-3,4-dihydroisoquinolin-1-yl)quinoline |
| D-718 | I.Ka-10 | 3-(4,4-difluoro-3,3-dimethyl-3,4-dihydroisoquinolin-1-yl)quinoline |
| D-719 | I.Ka-10 | 3-(4,4,5-trifluoro-3,3-dimethyl-3,4-dihydroisoquinolin-1-yl)quinoline |
| D-720 | I.Ka-10 | 9-fluoro-2,2-dimethyl-5-(3-quinolyl)-3H 1,4-benzoxazepine |
| D-721 | I.Ka-11 | Pyraclostrobin |
| D-722 | I.Ka-11 | Azoxystrobin |
| D-723 | I.Ka-11 | Trifloxystrobin |
| D-724 | I.Ka-11 | Picoxystrobin |
| D-725 | I.Ka-11 | Fluoxastrobin |
| D-726 | I.Ka-11 | Dimoxystrobin |
| D-727 | I.Ka-11 | Kresoxim-methyl |
| D-728 | I.Ka-11 | (2E,3Z)-5-[[1-(2,4-dichlorophenyl)-1H-pyrazol-3-yl]oxy]-2- |
| | | (methoxyimino)-N,3-dimethyl-pent-3-enamide |
| D-729 | I.Ka-11 | (2E,3Z)-5-[[1-(4-chlorophenyl)-1H-pyrazol-3-yl]oxy]-2- |
| | | (methoxyimino)-N,3-dimethyl-pent-3-enamide |
| D-730 | I.Ka-11 | [(3S,6S,7R,8R)-8-benzyl-3-[(3-acetoxy-4 methoxy-pyridine-2- |
| | | carbonyl)amino]-6-methyl-4,9-dioxo-1,5-dioxonan-7-yl] 2 |
| | | methylpropanoate |
| D-731 | I.Ka-11 | [(3S,6S,7R,8R)-8-benzyl-3-[[3-(acetoxymethoxy)-4-methoxy- |
| | | pyridine-2 carbonyl]amino]-6-methyl-4,9-dioxo-1,5-dioxonan-7-yl] 2 |
| | | methylpropanoate |
| D-732 | I.Ka-11 | [(3S,6S,7R,8R)-8-benzyl-3-[(3-isobutoxycarbonyloxy-4-methoxy- |
| | | pyridine-2 carbonyl)amino]-6-methyl-4,9-dioxo-1,5-dioxonan-7-yl] 2- |
| | | methylpropanoate |
| D-733 | I.Ka-11 | [(3S,6S,7R,8R)-8-benzyl-3-[[3-(1,3-benzodioxol-5-ylmethoxy)-4- |
| | | methoxy-pyridine-2-carbonyl]amino]-6-methyl-4,9-dioxo-1,5- |
| | | dioxonan-7-yl] 2-methylpropanoate |
| D-734 | I.Ka-11 | (3S,6S,7R,8R)-3-[[(3-hydroxy-4-methoxy-2- |
| | | pyridinyl)carbonyl]amino]-6 methyl-4,9-dioxo-8-(phenylmethyl)-1,5- |
| | | dioxonan-7-yl 2-methylpropanoate |
| D-735 | I.Ka-11 | Fluxapyroxad |
| D-736 | I.Ka-11 | Boscalid |
| D-737 | I.Ka-11 | Bixafen |
| D-738 | I.Ka-11 | Isopyrazam |
| D-739 | I.Ka-11 | Benzovindiflupyr |
| D-740 | I.Ka-11 | Fluopyram |
| D-741 | I.Ka-11 | N-[(5-chloro-2-isopropyl-phenyl)methyl]-N-cyclopropyl-5-fluoro-1,3- |
| | | dimethyl-pyrazole-4-carboxamide |
| D-742 | I.Ka-11 | Sedaxane |
| D-743 | I.Ka-11 | Penflufen |
| D-744 | I.Ka-11 | N-[2-(2,4-dichlorophenyl)-2-methoxy-1-methyl-ethyl]-3- |
| | | (difluoromethyl)-1-methyl-pyrazole-4-carboxamide |
| D-745 | I.Ka-11 | 3 (difluoromethyl)-1-methyl-N-(1,1,3-trimethylindan-4-yl)pyrazole-4- |
| | | carboxamide |
| D-746 | I.Ka-11 | 3 (trifluoromethyl)-1-methyl-N-(1,1,3-trimethylindan-4-yl)pyrazole-4- |
| | | carboxamide |
| D-747 | I.Ka-11 | 1,3-dimethyl-N-(1,1,3-trimethylindan-4-yl)pyrazole-4-carboxamide |
| D-748 | I.Ka-11 | 3-(trifluorometh¬yl)-1,5-dimethyl-N-(1,1,3-trimethylindan-4- |
| | | yl)pyrazole-4-carboxamide |
| D-749 | I.Ka-11 | 1,3,5-trimethyl-N-(1,1,3-trimethylindan-4-yl)pyrazole-4-carboxamide |
| D-750 | I.Ka-11 | 1-[3-chloro-2-[[[1-(4-chlorophenyl)-1H-pyrazol-3- |
| | | yl]oxy]methyl]phenyl]-1,4-dihydro-4-methyl-5H-tetrazol-5-one |
| D-751 | I.Ka-11 | Ametoctradin |
| D-752 | I.Ka-11 | epoxiconazole |
| D-753 | I.Ka-11 | metconazole |
| D-754 | I.Ka-11 | prothioconazole |
| D-755 | I.Ka-11 | difenoconazole |
| D-756 | I.Ka-11 | fluquinconazole |
| D-757 | I.Ka-11 | propiconazole |
| D-758 | I.Ka-11 | tebuconazole |
| D-759 | I.Ka-11 | 2-[2-chloro-4-(4-chlorophenoxy)phenyl]-1 (1,2,4-triazol-1-yl)pentan- |
| | | 2-ol |
| D-760 | I.Ka-11 | 1-[4-(4-chlorophenoxy)-2-(trifluoromethyl)phenyl]-1 cyclopropyl-2- |
| | | (1,2,4-triazol-1-yl)ethanol |
| D-761 | I.Ka-11 | 2-[4-(4-chlorophenoxy)-2-(trifluoromethyl)phenyl]-1-(1,2,4-triazol-1- |
| | | yl)butan-2-ol |
| D-762 | I.Ka-11 | 2-[2-chloro-4-(4-chlorophenoxy)phenyl]-1-(1,2,4-triazol-1-yl)butan- |
| | | 2-ol |
| D-763 | I.Ka-11 | 2-[4-(4-chlorophenoxy)-2-(trifluoromethyl)phenyl]-3-methyl-1-(1,2,4- |
| | | triazol-1-yl)butan-2-ol |
| D-764 | I.Ka-11 | 2-[4-(4-chlorophenoxy)-2-(trifluoromethyl)phenyl]-1-(1,2,4-triazol-1- |
| | | yl)propan-2-ol |
| D-765 | I.Ka-11 | 2-[2-chloro-4-(4-chlorophenoxy)phenyl]-3-methyl-1-(1,2,4-triazol-1- |
| | | yl)butan-2-ol |
| D-766 | I.Ka-11 | 2-[4-(4-chlorophenoxy)-2-(trifluoromethyl)phenyl]-1-(1,2,4-triazol-1- |
| | | yl)pentan-2-ol |
| D-767 | I.Ka-11 | 2-[4-(4-fluorophenoxy)-2-(trifluoromethyl)phenyl]-1-(1,2,4-triazol-1- |
| | | yl)propan-2-ol |
| D-768 | I.Ka-11 | 2-[2-chloro-4-(4-chlorophenoxy)phenyl]-1-(1,2,4-triazol-1-yl)pent-3- |
| | | yn-2-ol |
| D-769 | I.Ka-11 | prochloraz |
| D-770 | I.Ka-11 | [3-(4-chloro-2-fluoro-phenyl)-5-(2,4-difluorophenyl)isoxazol-4-yl]-(3- |
| | | pyridyl)methanol |
| D-771 | I.Ka-11 | fenpropimorph |
| D-772 | I.Ka-11 | Metalaxyl |
| D-773 | I.Ka-11 | Benalaxyl |
| D-774 | I.Ka-11 | Thiophanate-methyl |
| D-775 | I.Ka-11 | Carbendazim |
| D-776 | I.Ka-11 | Metrafenone |
| D-777 | I.Ka-11 | Pyrimethanil |
| D-778 | I.Ka-11 | Iprodione |
| D-779 | I.Ka-11 | Vinclozolin |
| D-780 | I.Ka-11 | Fludioxonil |
| D-781 | I.Ka-11 | dimethomorph |
| D-782 | I.Ka-11 | oxathiapiprolin |
| D-783 | I.Ka-11 | metiram |
| D-784 | I.Ka-11 | mancozeb |
| D-785 | I.Ka-11 | chlorothalonil |
| D-786 | I.Ka-11 | dithianon |
| D-787 | I.Ka-11 | Dipymetitrone |
| D-788 | I.Ka-11 | prohexadione-calcium |
| D-789 | I.Ka-11 | 3-(5-fluoro-3,3,4,4-tetramethyl-3,4-dihydroisoquinolin-1-yl)quinoline |
| D-790 | I.Ka-11 | 3-(4,4-difluoro-3,3-dimethyl-3,4-dihydroisoquinolin-1-yl)quinoline |
| D-791 | I.Ka-11 | 3-(4,4,5-trifluoro-3,3-dimethyl-3,4-dihydroisoquinolin-1-yl)quinoline |
| D-792 | I.Ka-11 | 9-fluoro-2,2-dimethyl-5-(3-quinolyl)-3H 1,4-benzoxazepine |

The active substances referred to as component 2, their preparation and their activity e.g. against harmful fungi is known (cf.: http://www.alanwood.net/pesticides/); these substances are commercially available. The compounds described by IUPAC nomenclature, their preparation and their pesticidal activity are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 11/028657, WO2012/168188, WO 2007/006670, WO 2011/77514; WO13/047749, WO 10/069882, WO 13/047441, WO 03/16303, WO 09/90181, WO 13/007767, WO 13/010862, WO 13/127704, WO 13/024009, WO 13/024010 and WO 13/047441, WO 13/162072, WO 13/092224, WO 11/135833).

The present invention furthermore relates to agrochemical compositions comprising a mixture of at least one compound I (component 1) and at least one further active substance useful for plant protection, e.g. selected from the groups A) to O) (component 2), in particular one further fungicide, e.g. one or more fungicide from the groups A) to K), as described above, and if desired one suitable solvent or solid carrier. Those mixtures are of particular interest, since many of them at the same application rate show higher efficiencies against harmful fungi. Furthermore, combating harmful fungi with a mixture of compounds I and at least one fungicide from groups A) to K), as described above, is more efficient than combating those fungi with individual compounds I or individual fungicides from groups A) to K).

By applying compounds I together with at least one active substance from groups A) to O) a synergistic effect can be obtained, i.e. more then simple addition of the individual effects is obtained (synergistic mixtures).

This can be obtained by applying the compounds I and at least one further active substance simultaneously, either jointly (e.g. as tank-mix) or seperately, or in succession, wherein the time interval between the individual applications is selected to ensure that the active substance applied first still occurs at the site of action in a sufficient amount at the time of application of the further active substance(s). The order of application is not essential for working of the present invention.

When applying compound I and a pesticide II sequentially the time between both applications may vary e.g. between 2 hours to 7 days. Also a broader range is possible ranging from 0.25 hour to 30 days, preferably from 0.5 hour to 14 days, particularly from 1 hour to 7 days or from 1.5 hours to 5 days, even more preferred from 2 hours to 1 day.

In the binary mixtures and compositions according to the invention the weight ratio of the component 1) and the component 2) generally depends from the properties of the active components used, usually it is in the range of from 1:10,000 to 10,000:1, often it is in the range of from 1:100 to 100:1, regularly in the range of from 1:50 to 50:1, preferably in the range of from 1:20 to 20:1, more preferably in the range of from 1:10 to 10:1, even more preferably in the range of from 1:4 to 4:1 and in particular in the range of from 1:2 to 2:1.

In still another embodiments of the binary mixtures and compositions, the weight ratio of the component 1) and the component 2) usually is in the range of from 1000:1 to 1:1, often in the range of from 100: 1 to 1:1, regularly in the range of from 50:1 to 1:1, preferably in the range of from 20:1 to 1:1, more preferably in the range of from 10:1 to 1:1, even more preferably in the range of from 4:1 to 1:1 and in particular in the range of from 2:1 to 1:1.

In still another embodiments of the binary mixtures and compositions, the weight ratio of the component 1) and the component 2) usually is in the range of from 1:1 to 1:1000, often in the range of from 1:1 to 1:100, regularly in the range of from 1:1 to 1:50, preferably in the range of from 1:1 to 1:20, more preferably in the range of from 1:1 to 1:10, even more preferably in the range of from 1:1 to 1:4 and in particular in the range of from 1:1 to 1:2.

In the ternary mixtures, i.e. compositions according to the invention comprising the component 1) and component 2) and a compound III (component 3), the weight ratio of component 1) and component 2) depends from the properties of the active substances used, usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:4 to 4:1, and the weight ratio of component 1) and component 3) usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:4 to 4:1.

Any further active components are, if desired, added in a ratio of from 20:1 to 1:20 to the component 1).

These ratios are also suitable for inventive mixtures applied by seed treatment.

### I. SYNTHESIS EXAMPLE

With due modification of the starting compounds, the procedures shown in the synthesis examples below were used to obtain further compounds I. The resulting compounds, together with physical data, are listed in Table I below.

HPLC-MS: HPLC-column Kinetex XB C18 1.7μ (50×2.1 mm); eluent: acetonitrile/water+0.1% TFA (5 gradient from 5:95 to 100:0 in 1.5 min at 60° C., flow gradient from 0.8 to 1.0 ml/min in 1.5 min). MS: Quadrupol Electrospray Ionisation, 80 V (positive mode).

### 1) 1-(5,6-dimethyl-3-pyridyl)-3,5-dimethyl-3-phenyl-4H-isoquinoline

2.41 g of trifluoromethane sulfonic acid was added dropwise to a mixture of 0.22 g (1.61 mmol) 2,3-dimethylpyridine-5-carbonitrile and 0.5 g (1.77 mmol) 2-(o-tolyl)-1-phenyl-propan-2-ol in 50 ml dichloroethane at 0-5° C. After 1 days at room temperature the reaction mixture was poured onto sodium carbonate solution, the organic layer was separated and the aqueous layer was extracted with dichloromethane. The combined organic extracts were extracted with sodium hydrogen carbonate and water, evaporated and the residue was purified via silica gel column chromatography with EtOAc/n-heptan mixtures to yield 0.133 g (80%) of the title compound as a brown oil.

¹H-NMR (CDCl3, δ in ppm): 8.6 (s, 1H); 7.8 (s, 1H); 7,5 (m, 2H); 7.5-7.2 (m, 4H); 7.0 (m 2H); 3.2 (d, 1H); 3.0, (d, 1H); 2.6 (s, 3H); 2.4-2.3 (m, 3H+3H); 1.6 (s, 3H). *HPLC-MS: Rt=0.926 min; m⁺+H=341.2

### 2) 5-Bromo-1-(5,6-dimethyl-3-pyridyl)-3,3-dimethyl-4H-isoquinoline

3.2 g of trifluoromethane sulfonic acid was added dropwise to a mixture of 0.56 g (4.2 mmol) 2,3-dimethylpyridine-5-carbonitrile and 0.9 g (4.2 mmol) 1-bromo-2-(2-methylprop-1-enyl)benzene in 20 ml dichloroethane at 0-5° C. After 90 minutes at room temperature the reaction mixture was poured onto sodium carbonate solution, the organic layer was separated and the aqueous layer was extracted with dichloromethane. The combined organic extracts were extracted with sodium hydrogen carbonate and water, evaporated and the residue was purified via silica gel chromatography with cycohexane/ethyl acetate mixtures to yield 0.65 g (45%) of the title compound as a light yellow oil.

¹H-NMR (CDCl₃, δ in ppm): 8.4 (s, 1H); 7.6 (m, 2H); 7.1 (m, 2H); 2.9 (s, 2H); 2.55 (s, 3H); 2.3 (s, 3H); 1.3 (s, 6H).

*HPLC-MS: Rₜ=0.840 min; m⁺+H=343

### 3) 5-Cyano-1-(5,6-dimethyl-3-pyridyl)-3,3-dimethyl-4H-isoquinoline

A mixture of 0.28 g (0.82 mmol) 5-bromo-1-(5,6-dimethyl-3-pyridyl)-3,3-dimethyl-4H-isoquinoline (ex. 1) and 0.15 g (1.63 mmol) CuCN in 20 ml N-methyl pyrrolidine was stirred over night at 130° C. Afterwards the reaction mixture was cooled to room temperature and diluted with water and methyl-t-butyl ether. A solid precipated and was filtered off. Subsequently the phases were separated and the aqueous layer was extracted twice with methyl-t-butylether. The combined organic layers were dried over magnesium sulfate and concentrated. The residue was purified via silica gel chromatography with cycohexane/ethyl acetate mixtures to yield 0.04 g (17%) of the title compound as a orange oil.

¹H-NMR (CDCl₃, 6 in ppm): 8.45 (s, 1H); 7.7 (d, 1H); 7.65 (s, 1H);7.45 (d, 1H); 7.35 (t, 1H); 3.05 (s, 2H); 2.55 (s, 3H); 2.35 (s, 3H); 1.3 (s, 6H).

*HPLC-MS: Rₜ=0.753 min; m⁺+H =290.1

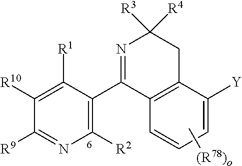

TABLE I

| | | | | | | | | | Mp [° C.]; HPLC-MS (Rt |
|---|---|---|---|---|---|---|---|---|---|
| No. | R¹ | R² | R³ | R⁴ | R⁹ | R¹⁰ | o | Y | [min], M⁺ + H); |
| I-1 | H | H | CH₃ | CH₃ | CH₃ | CH₃ | 0 | Br | 0.840 min; |
| | | | | | | | | | M⁺ + H = 343 |
| I-2 | H | H | CH₃ | CH₃ | CH₃ | CH₃ | 0 | CH₃ | 0.791 min; |
| | | | | | | | | | M⁺ + H = 279.1 |
| I-3 | H | H | CH₃ | CH₃ | CH₃ | CH₃ | 0 | CF₃ | 0.886 min; |
| | | | | | | | | | M⁺ + H = 333.1 |
| I-4 | H | H | CH₃ | CH₃ | CH₃ | CH₃ | 0 | CH(CH₃)₂ | 0.881 min; |
| | | | | | | | | | M⁺ + H = 307.3 |
| I-5 | H | H | CH₃ | CH₃ | CH₃ | CH₃ | 0 | C₂H₅ | 0.818 min; |
| | | | | | | | | | M⁺ + H = 293.1 |
| I-6 | H | H | CH₃ | CH₃ | CH₃ | CH₃ | 0 | CHF₂ | 0.814 min; |
| | | | | | | | | | M⁺ + H = 314.9 |
| I-7 | H | H | CH₃ | CH₃ | CH₃ | CH₃ | 0 | CN | 0.753 min; |
| | | | | | | | | | M⁺ + H = 290.1 |
| I-8 | H | H | CH₃ | CH₃ | CH₃ | CH₃ | 0 | OCHF₂ | 0.810 min; |
| | | | | | | | | | M⁺ + H = 331.2 |
| I-9 | H | H | CH₃ | C₅H₆ | CH₃ | CH₃ | 0 | CH₃ | 0.926 min; |
| | | | | | | | | | M⁺ + H = 341.2 |
| I-10 | H | H | 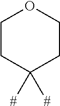 | | CH₃ | CH₃ | 0 | Br | 0.847 min; M⁺ + H = 385 |
| I-11 | H | H | CH₃ | CH₃ | Br | Br | 0 | Br | 1.086 min; |
| | | | | | | | | | M⁺ + H = 474.8 |
| I-12 | H | H | CH₃ | CH₃ | CHF₂ | Br | 0 | Br | 1.071 min; |
| | | | | | | | | | M⁺ + H = 444.9 |
| I-13 | H | H | CH₃ | CH₃ | Cl | Br | 0 | Br | 0.940 min; |
| | | | | | | | | | M⁺ + H = 379.0 |
| I-14 | H | H | CH₃ | CH₃ | CHF₂ | OCH₃ | 0 | Br | 0.918 min; |
| | | | | | | | | | M⁺ + H = 397.0 |
| I-15 | H | H | CH₃ | 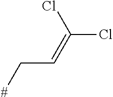 | CH₃ | CH₃ | 0 | Cl | 8.4 (br s, 1H); 7.6 (s, 1H); 7.1-7.0 (dd, 1H); 6.0 (dd, 1H); 2.8 (s, 2H); 2.5 (s, 3H); 2.4-2.3 (m, 2H); 2.25 (s, 3H); 1.3 (s, 3H) |
| I-16 | H | H | CH₃ | CH₃ | 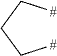 | | 0 | Br | 0.849 min; M⁺ + H = 357.1 |
| I-17 | H | H | CH₃ | CH₃ | CH₃ | CH₃ | 0 | Cl | 0.791 min; |
| | | | | | | | | | M⁺ + H = 299.1 |

### II. BIOLOGICAL TESTS

Microtest

The active compounds were formulated separately as a stock solution having a concentration of 10000 ppm in dimethyl sulfoxide.

### EXAMPLE 1

### Activity Against the Grey Mold Botrytis cinerea in the Microtiterplate Test

The stock solutions were mixed according to the ratio, pipetted onto a micro titer plate (MTP) and diluted with water to the stated concentrations. A spore suspension of Botrci cinerea in an aqueous biomalt or yeast-bactopeptone-sodiumacetate solution was then added. The plates were placed in a water vapor-saturated chamber at a temperature of 18° C. Using an absorption photometer, the MTPs were measured at 405 nm 7 days after the inoculation.

In this test, the samples which had been treated with 32 ppm of the active substance from examples 1-1, 1-2, 1-3, 1-5, 1-6, 1-9 and 1-16 respectively, showed up to at most 18% growth of the pathogen.

### EXAMPLE 2

### Activity Against Rice Blast Pyricularia oryzae in the Microtiterplate Test

The stock solutions were mixed according to the ratio, pipetted onto a micro titer plate (MTP) and diluted with water to the stated concentrations. A spore suspension of Pyricularia oryzae in an aqueous biomalt or yeast-bactopeptone-glycerine solution was then added. The plates were placed in a water vapor-saturated chamber at a temperature of 18° C. Using an absorption photometer, the MTPs were measured at 405 nm 7 days after the inoculation.

In this test, the samples which had been treated with 32 ppm of the active substance from examples 1-1, 1-2, 1-3, 1-4, 1-5, 1-6, 1-8 and 1-9 respectively, showed up to at most 10% growth of the pathogen.

### EXAMPLE 3

### Activity Against Leaf Blotch on Wheat Caused by Septoria tritici

The stock solutions were mixed according to the ratio, pipetted onto a micro titer plate (MTP) and diluted with water to the stated concentrations. A spore suspension of Septoria tritici in an aqueous biomalt or yeast-bactopeptone-glycerine solution was then added. The plates were placed in a water vapor-saturated chamber at a temperature of 18° C. Using an absorption photometer, the MTPs were measured at 405 nm 7 days after the inoculation.

In this test, the samples which had been treated with 32 ppm of the active substance from examples 1-1, 1-2, 1-3, 1-4, 1-5, 1-6 and 1-16 respectively, showed up to at most 15% growth of the pathogen.

The measured parameters were compared to the growth of the active compound-free control variant (100%) and the fungus-free and active compound-free blank value to determine the relative growth in % of the pathogens in the respective active compounds.

Green House

The spray solutions were prepared in several steps:

The stock solution were prepared: a mixture of acetone and/or dimethylsulfoxide and the wetting agent/emulsifier Wettol, which is based on ethoxylated alkylphenoles, in a relation (volume) solvent-emulsifier of 99 to 1 was added to 25 mg of the compound to give a total of 5 ml. Water was then added to total volume of 100 ml.

This stock solution was diluted with the described solvent-emulsifier-water mixture to the given concentration.

### EXAMPLE 1

### Preventative Fungicidal Control of Apple Scrab Venturia inaequalis

Young seedlings of apple plants were sprayed to run-off with an aqueous suspension, containing the concentration of active ingredient or their mixture mentioned in the table below. The next day the plants were inoculated with an aqueous solution containing the spore suspension of Venturia inaequalis. Then the plants were immediately transferred to a humid chamber. After 1 day at 22 to 24° C. and a relative humidity close to 100% the plants were transferred to a chamber with 22-24° C. and a relative humidity of 70%. After 12 days the extent of fungal attack on the leaves was visually assessed as % diseased leaf area.

In this test, plants which had been treated with 63 ppm of the active substance from examples I-1, I-3, I-7, I-12, I-14 and I-16 respectively, showed up to at most 10% diseased leaf area compated to 90% diseased leaf area by untreated plants.

### III. COMPARATIVE EXAMPLES

M icrotests

### EXAMPLE 1

### Activity Against the Grey Mold Botrytis cinerea in the Microtiter Plate Test

The stock solutions were mixed according to the ratio, pipetted onto a micro titer plate (MTP) and diluted with water to the stated concentrations. A spore suspension of Botrci cinerea in an aqueous biomalt or yeast-bactopeptone-sodiumacetate solution was then added. The plates were placed in a water vapor-saturated chamber at a temperature of 18° C. Using an absorption photometer, the MTPs were measured at 405 nm 7 days after the inoculation.

### EXAMPLE 2

### Activity Against Leaf Blotch on Wheat Caused by Septoria tritici

The stock solutions were mixed according to the ratio, pipetted onto a micro titer plate (MTP) and diluted with water to the stated concentrations. A spore suspension of Septoria tritici in an aqueous biomalt or yeast-bactopeptone-glycerine solution was then added. The plates were placed in a water vapor-saturated chamber at a temperature of 18° C. Using an absorption photometer, the MTPs were measured at 405 nm 7 days after the inoculation.

| | Growth (%) at | Growth (%) at |
|---|---|---|
| | 32 ppm | 32 ppm |
| Nr. | Botottis cinerea | Septona triad |
| 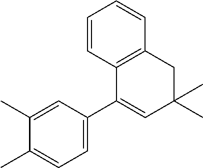 | 84 | 93 |
| (SdT) | | |
| I-1 | 0 | 0 |
| I-5 | 4 | 1 |
| I-6 | 4 | 13 |
| I-2 | 18 | 15 |

### EXAMPLE 3

### Activity Against Rice Blast Pyricularia oryzae in the Microtiter Plate Test

The stock solutions were mixed according to the ratio, pipetted onto a micro titer plate (MTP) and diluted with water to the stated concentrations. A spore suspension of Pyricularia oryzae in an aqueous biomalt or yeast-bactopeptone-glycerine solution was then added. The plates were placed in a water vapor-saturated chamber at a temperature of 18° C. Using an absorption photometer, the MTPs were measured at 405 nm 7 days after the inoculation.

### EXAMPLE 4

### Activity Against Early Blight Caused by Alternaria solani

The stock solutions were mixed according to the ratio, pipetted onto a micro titer plate (MTP) and diluted with water to the stated concentrations. A spore suspension of Alternaria solani in an aqueous biomalt or yeast-bactopeptone-glycerine solution was then added. The plates were placed in a water vapor-saturated chamber at a temperature of 18° C. Using an absorption photometer, the MTPs were measured at 405 nm 7 days after the inoculation.

### EXAMPLE 5

### Activity Against Wheat Leaf Spots Caused by Leptosphaeria nodorum

The stock solutions were mixed according to the ratio, pipetted onto a micro titer plate (MTP) and diluted with water to the stated concentrations. A spore suspension of Leptosphaena nodorum in an aqueous biomalt or yeast-bactopeptone-glycerine solution was then added. The plates were placed in a water vapor-saturated chamber at a temperature of 18° C. Using an absorption photometer, the MTPs were measured at 405 nm 7 days after the inoculation.

The measured parameters were compared to the growth of the active compound-free control variant (100%) and the fungus-free and active compound-free blank value to determine the relative growth in % of the pathogens in the respective active compounds.

| | Growth (%) | Growth (%) at | |
|---|---|---|---|
| | at 31 ppm | 31 ppm | Growth (%) at 31 ppm |
| Structure | Pyricularia | Alternaria solanioryzae | Leptosphaeria nodorum |
| 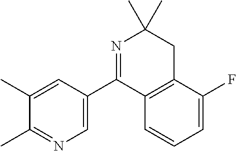 [SMILES: Cc1cc(C2=NC(C)(C)Cc3c(F)cccc32)cnc1C] | | | 39 |
| 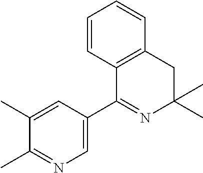 | 23 | 47 | 72 |
| I-17 | 2 | 0 | 10 |

Green House

### EXAMPLE 1

### Control of Culm Rot on Pearl Millet Caused by Fusarium culmorum

Pot-grown pearl millet seedlings were sprayed to run-off with an aqueous suspension, containing the concentration of active ingredient or their mixture as described below.The plants were allowed to air-dry. Seven days later the plants were inoculated with an spore suspension of Fusarium culmorum in a aqueous biomalt solution. Then the trial plants were immediately transferred to a humid chamber. After 6 days at 23-25° C. and a relative humidity close to 100% the extent of fungal attack on the leaves was visually assessed as % diseased leaf area.

| Nr. | Disease (%) at 63 ppm |
|---|---|
| 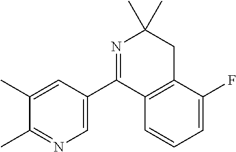 [SMILES: Cc1cc(C2=NC(C)(C)Cc3c(F)cccc32)cnc1C] | 88 |
| (SdT) | |
| I-1 | 53 |
| I-5 | 30 |
| I-6 | 36 |
| I-2 | 65 |
| Untreated control | 90 |

1-15. (canceled)

16. A compound of formula I 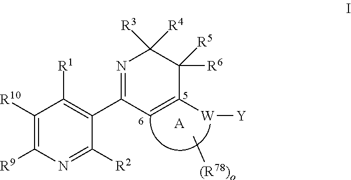
wherein
R¹ is in each case independently selected from the group consisting of H, halogen, OH, CN, NO₂, SH, NH₂, NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains one, two or three heteroatoms selected from the group consisting of N, O and S; and wherein
R^{x} is selected from the group consisting of C₁-C₄-alkyl, unsubstituted aryl and aryl that is substituted with substituents R^{x1} independently selected from C₁-C₄-alkyl;
wherein the acyclic moieties of R¹ are unsubstituted or substituted by groups R^{1a} which independently of one another are selected from the group consisting of:
R^{1a} is selected from the group consisting of halogen, OH, CN, C₁-C₆-alkoxy, C₃-C₆-cycloalkyl, C₃-C₆-halocycloalkyl, C₁-C₄-haloalkoxy, C₁-C₆-alkylthio and phenoxy, wherein the phenyl group is unsubstituted or substituted by substituents R^{11a} selected from the group consisting of halogen, OH, C₁-C₄-alkyl, C₁-C₄-haloalkyl, C₁-C₄-alkoxy and C₁-C₄-haloalkoxy;
wherein the cycloalkyl, heteroaryl and aryl moieties of R¹ are unsubstituted or substituted by groups R^{1b} which independently of one another are selected from the group consisting of:
R^{1b} is selected from the group consisting of halogen, OH, CN, C₁-C₄-alkyl, C₁-C₄-alkoxy, C₁-C₄-haloalkyl, C₃-C₆-cycloalkyl, C₃-C₆-halocycloalkyl, C₁-C₄-haloalkoxy and C₁-C₆-alkylthio;
R² is in each case independently selected from the substituents as defined for R¹, wherein the possible substituents for R² are R^{e}a and R^{2b}, respectively, which correspond to R^{1a} and R^{1b}, respectively;
R³,R⁴ are independently selected from the group consisting of halogen, OH, CN, NO₂, SH, C₁-C₆-alkylthio, C₁-C₆-haloalkylthio, NH₂, NH(C₁-C₄-alkyl), N(C₁-C₄-alkyl)₂, NH—SO₂—R^{x}, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, CH(═O), C(═O)C₁-C₆-alkyl, C(═O)OC₁-C₆-alkyl, C(═O)NHC₁-C₆-alkyl, C(═O)N(C₁-C₆-alkyl)₂, CR^{x}═NR^{x}, saturated or partially unsaturated three-, four-, five-, six-, seven-, eight-, nine-, or ten-membered carbocycle or heterocycle, wherein in each case one or two CH₂ groups of the carbo- and heterocycle may be replaced by a group independently selected from the group consisting of C(═O) and C(═S), five- or six-membered heteroaryl and aryl; wherein the heterocycle and the heteroaryl contain independently one, two, three or four heteroatoms selected from N, O and S; or
R³, R⁴ together with the carbon atom to which they are bound form saturated, partially unsaturated three-, four-, five-, six-, seven-, eight-, nine-, or ten-membered carbo- or heterocycle, wherein the heterocycle contains one, two, three or four heteroatoms selected from the group consisting of N, O and S, wherein the heteroatom N may carry one substituent selected from the group consisting of C₁-C₄-alkyl, C₁-C₄-haloalkyl and SO₂Ph, wherein Ph is unsubstituted phenyl or phenyl that is substituted by one, two or three substituents selected from the group consisting of C₁-C₄-alkyl, halogen, C₁-C₄-haloalkyl, C₁-C₄-alkoxy and C₁-C₄-haloalkoxy, and CN; and wherein the heteroatom S may be in the form of its oxide SO or SO₂, and wherein the carbo- or heterocycle is unsubstituted or carries one, two, three or four substituents R³⁴ independently selected from the group consisting of halogen, OH, CN, NO₂, SH, NH₂, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₄-alkoxy-C₁-C₄-alkyl, phenyl and phenoxy, wherein the phenyl groups are unsubstituted or carry one, two, three, four or five substituents R^{34a} selected from the group consisting of halogen, OH, C₁-C₄-alkyl, C₁-C₄-haloalkyl, C₁-C₄-alkoxy, haloalkoxy and CN; and wherein in each case one or two CH₂ groups of the carbo- or heterocycle may be replaced by a group independently selected from the group consisting of C(═O) and C(═S); and
wherein the acyclic moieties of R³ and R⁴ are independently unsubstituted or substituted by groups R^{3a} or R^{4a}, respectively, which independently of one another are selected from the group consisting of:
R^{3a}, R^{4a} is selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, C₁-C₆-alkoxy, C₃-C₆-cycloalkyl, C₃-C₆-halocycloalkyl, C₁-C₄-haloalkoxy, C₁-C₆-alkylthio, CH(═O), C(═O)C₁-C₆-alkyl, C(═O)OC₁-C₆-alkyl, C(═O)NHC₁-C₆-alkyl, C(═O)N(C₁-C₆-alkyl)₂, CR^{x}═NR^{x}, a saturated or partially unsaturated three-, four-, five-, six-, seven-, eight-, nine-, or ten-membered carbo or heterocycle, wherein in each case one or two CH₂ groups of the carbo- and heterocycle may be replaced by a group independently selected from the group consisting of C(═O) and C(═S), an aryl and phenoxy, wherein the aryl and phenyl groups are independently unsubstituted or substituted with substituents selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₆-alkylthio, C₁-C₄-alkyl, C₁-C₄-haloalkyl, C₁-C₄-alkoxy and C₁-C₄-haloalkoxy;
wherein the carbocyclic, heterocyclic, heteroaryl and aryl moieties of R³ and R⁴ are independently unsubstituted or substituted with identical or different groups R^{3b} or R^{4b}, respectively, which independently of one another are selected from:
R^{3b}, R^{4b} is selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, C₁-C₄-alkyl, C₁-C₄-alkoxy, C₁-C₄-haloalkyl, C₃-C₆-cycloalkyl, C₃-C₆-halocycloalkyl, C₁-C₄-haloalkoxy, C₁-C₆-alkylthio, C₁-C₆-haloalkylthio, C₁-C₄-alkoxy-C₁-C₄-haloalkyl, phenyl and phenoxy, wherein the phenyl groups are unsubstituted or carry one, two, three, four or five substituents selected from the group consisting of halogen, OH, C₁-C₄-alkyl, C₁-C₄-haloalkyl, C₁-C₄-alkoxy and C₁-C₄-haloalkoxy;
and wherein R^{x} is as defined above; and
R⁵ is H;
R⁶ is H;
ring A-W—Y is selected from below group: 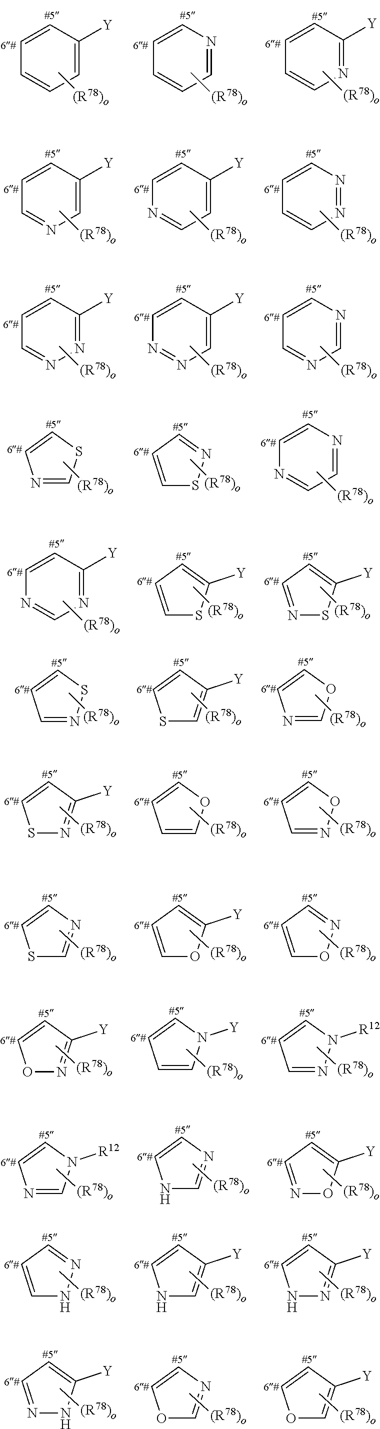 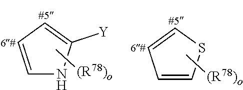
wherein, the positions of the rings marked with “#” represents the connection points (carbon atoms 5″ and 6″ in formula I) with the remaining skeleton of the compounds of formula I; wherein
o is 0, 1, 2 or 3; and
R⁷⁸ are independently selected from the group consisting of halogen, OH, CN, NO₂, SH, NH₂, NH(C₁-C₄-alkyl), N(C₁-C₄-alkyl)₂, NH(C(═O)C₁-C₄-alkyl), N(C(═O)C₁-C₄-alkyl)₂, NH—SO₂—R^{x}, CH(═O), C(═O)C₁-C₆-alkyl, C(═O)NH(C₁-C₆-alkyl), CR′═NOR″, C₁-C₆-alkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₂-C₆-alkenyloxy, C₂-C₆-alkynyloxy, C₃-C₆-cycloalkyl, C₃-C₆-cycloalkenyl, three-, four-, five- or six-membered saturated or partially unsaturated heterocycle, five- or six-membered heteroaryl and phenyl; wherein the heterocycle or heteroaryl contains one, two or three heteroatoms selected from the group consisting of N, O and S; wherein R′ and R″ are independently selected from the group consisting of H, C₂-C₆-alkenyl, C₂-C₆-alkynyl, C₃-C₆-cycloalkyl, saturated or partially unsaturated three-, four-, five-, six-, seven-, eight-, nine-, or ten-membered heterocycle, five- or six-membered heteroaryl and aryl; wherein the heterocycle or heteroaryl contains one, two or three heteroatoms selected from the group consisting of N, O and S, and wherein R′ and R″ are independently unsubstituted or substituted by R′″ which is independently selected from the group consisting of halogen, OH, CN, NO₂, SH, NH₂, NH(C₁-C₄-alkyl), N(C₁-C₄-alkyl)₂, NH—SO₂—R^{x}, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₃-C₆-cycloalkyl, C₃-C₆-halogencycloalkyl and phenyl; and wherein R^{x} is defined above; and
wherein the acyclic moieties of R⁷⁸ are unsubstituted or substituted by R^{78a} which independently of one another are selected from the group consisting of:
R^{78a} is selected from the group consisting of halogen, OH, CN, C₁-C₆-alkoxy, C₃-C₆-cycloalkyl, C₃-C₆-cycloalkenyl, C₃-C₆-halocycloalkyl, C₃-C₆-halocycloalkenyl, C₁-C₄-haloalkoxy, C₁-C₆-alkylthio, five- or six-membered heteroaryl, phenyl and phenoxy, wherein the heteroaryl, phenyl and phenoxy group is unsubstituted or substituted by R^{78aa} selected from the group consisting of halogen, OH, C₁-C₄-alkyl, C₁-C₄-haloalkyl, C₁-C₄-alkoxy and C₁-C₄-haloalkoxy;
wherein the alicyclic, phenyl, heterocyclic and heteroaryl moieties of R⁷⁸ are unsubstituted or substituted by R^{78b} which independently of one another are selected from the group consisting of:
R^{78b} is selected from the group consisting of halogen, OH, CN, C₁-C₄-alkyl, C₁-C₄-alkoxy, C₁-C₄-haloalkyl, C₃-C₆-cycloalkyl, C₃-C₆-halocycloalkyl, C₁-C₄-haloalkoxy, and C₁-C₆-alkylthio;
Y is independently selected from the group consisting of F, Cl, Br, I, cyano, nitro, C₁-C₆-alkyl, C₁-C₆-haloalkyl, C₂-C₆-alkenyl, C₁-C₆-haloalkoxy, C₂-C₆-alkynyl, C₃-C₆-cycloalkyl, C₃-C₆-cycloalkenyl, —O(Y¹), —S(O)_{z}(Y²), —N(Y³)(Y⁴), C₁-C₆-haloalkyl, C₂-C₆-haloalkenyl, C₂-C₆-haloalkynyl, —CO(Y⁵), and —C(Y⁶)═NO(Y⁷); wherein the acyclic moieties of Y are unsubstituted or substituted by R^{78a} and the alicyclic, phenyl and heteroaryl moieties of Y are unsubstituted or substituted by R^{78b};
z is 0, 1 or 2;
Y¹ is selected from the group consisting of H, C₁-C₆-alkyl, C₂-C₆-alkenyl, C₂-C₆-alkynyl, C₃-C₆-cycloalkyl, cycloalkenyl, C₁-C₆-haloalkyl, C₂-C₆-haloalkenyl, C₂-C₆-haloalkynyl, phenyl and heteroaryl;
Y² is selected from the group consisting of H, C₁-C₆-alkyl, C₂-C₆-alkenyl, C₂-C₆-alkynyl, cycloalkyl, cycloalkenyl, C₁-C₆-haloalkyl, C₂-C₆-haloalkenyl, C₂-C₆-haloalkynyl, phenyl and heteroaryl;
Y³, Y⁴ are selected from the group consisting of H, C₁-C₆-alkyl, C₂-C₆-alkenyl, C₂-C₆-alkynyl, cycloalkyl, cycloalkenyl, C₁-C₆-haloalkyl, C₂-C₆-haloalkenyl, C₂-C₆-haloalkynyl, phenyl, heteroaryl and CO(Y³¹);
Y³¹ is selected from the group consisting of H, C₁-C₆-alkyl, C₂-C₆-alkenyl, C₂-C₆-alkynyl, cycloalkyl, cycloalkenyl, C₁-C₆-haloalkyl, C₂-C₆-haloalkenyl, C₂-C₆-haloalkynyl, phenyl, heteroaryl, —O(Y³¹¹) and —N(Y³¹²)(Y³¹³);
Y³¹¹ is selected from the group consisting of C₁-C₆-alkyl, C₂-C₆-alkenyl, C₂-C₆-alkynyl, cycloalkyl, cycloalkenyl, C₁-C₆-haloalkyl, C₂-C₆-haloalkenyl, C₂-C₆-haloalkynyl, phenyl and heteroaryl;
Y³¹², Y³¹³ are selected from the group consisting of H, C₁-C₆-alkyl, C₂-C₆-alkenyl, C₂-C₆-alkynyl, cycloalkyl, cycloalkenyl, C₁-C₆-haloalkyl, C₂-C₆-haloalkenyl, C₂-C₆-haloalkynyl, phenyl and heteroaryl;
Y⁵ is selected from the group consisting of H, C₁-C₆-alkyl, C₂-C₆-alkenyl, C₂-C₆-alkynyl, cycloalkyl, cycloalkenyl, C₁-C₆-haloalkyl, C₂-C₆-haloalkenyl, C₂-C₆-haloalkynyl, phenyl and heteroaryl;
Y⁶ is selected from the group consisting of H, C₁-C₆-alkyl, C₂-C₆-alkenyl, C₂-C₆-alkynyl, cycloalkyl, cycloalkenyl, C₁-C₆-haloalkyl, C₂-C₆-haloalkenyl, C₂-C₆-haloalkynyl, phenyl and heteroaryl;
Y⁷ is selected from the group consisting of H, C₁-C₆-alkyl, C₂-C₆-alkenyl, C₂-C₆-alkynyl, cycloalkyl, cycloalkenyl, C₁-C₆-haloalkyl, C₂-C₆-haloalkenyl, C₂-C₆-haloalkynyl, phenyl and heteroaryl;
R⁹, R¹⁰ are independently selected from the group consisting of H, halogen, CN, NO₂, N(R⁹¹)(R⁹²), S(R⁹³), S(O)_{z94}(R⁹⁴), 0(R⁹⁵), C₁-C₆-alkyl, C₂-C₆-alkenyl, C₂-C₆-alkynyl, cycloalkyl, CO-(R⁹⁶) and CS—(R⁹⁶);
R⁹¹, R⁹² are independently selected from the group consisting of H, alkyl, alkenyl, alkynyl , cycloalkyl, CO—R(⁹¹¹), and S(O)_{z91}R⁹¹²;
R⁹¹¹ is selected from the group the group consisting of H and R⁹¹²;
R⁹¹² is independently selected from the group consisting of alkyl, alkenyl, alkynyl, cycloalkyl, O—R⁹¹¹¹, and N(R9112)(R⁹¹¹³);
R⁹¹¹¹ is selected from the group consisting of alkyl, alkenyl, alkynyl alkenyl, and cycloalkyl;
R⁹¹¹², R⁹¹¹³ are independently selected from the group consisting of H, alkyl, alkenyl, alkynyl, and cycloalkyl;
z⁹¹ is 1 or 2;
R⁹³ is selected from the group consisting of H, alkyl, alkenyl, alkynyl, and cycloalkyl;
R⁹⁴ is independently selected from the group consisting of alkyl, alkenyl, alkynyl, cycloalkyl, O—R⁹⁴¹, and N(R942)(R943);
R⁹⁴¹ is independently selected from the group consisting of alkyl, alkenyl, alkynyl, and cycloalkyl;
R⁹⁴², R⁹⁴³ are independently selected from the group consisting of H and R⁹⁴¹;
z⁹⁴ is 1 or 2;
R⁹⁵ is independently selected from the group consisting of H, alkyl, alkenyl, alkynyl, cycloalkyl, carbonyl-R⁹⁵¹, and S(O)_{z95}R⁹⁵²;
R⁹⁵¹ is selected from the group consisting of H and R⁹⁵²;
R⁹⁵² is independently selected from the group consisting of alkyl, alkenyl, alkynyl, cycloalkyl, O—R⁹⁵²¹, and N(R⁹⁵²²)(R⁹⁵²³);
R⁹⁵²¹ is independently selected from the group consisting of alkyl, alkenyl, alkynyl, and cycloalkyl;
R9522, R⁹⁵²³ is independently selected from the group consisting of H and R⁹⁵²¹;
z⁹⁵ is 1 or 2;
R⁹⁶ is independently selected from the group consisting of H, alkyl, alkenyl, alkynyl, cycloalkyl, O—R⁹⁶¹, and N(R⁹⁶²)(R⁹⁶³);
R⁹⁶¹ is independently selected from the group consisting of H, alkyl, alkenyl, alkynyl, and cycloalkyl;
R⁹⁶², R⁹⁶³ are independently selected from the group consisting of H, alkyl, alkenyl, alkynyl, and cycloalkyl;
wherein the acyclic moieties of R⁹, R¹⁰ are unsubstituted or substituted by identical or different groups R^{9a}, wherein:
R^{9a} independently of one another are selected from the group consisting of halogen, OH, CN, C₁-C₆-alkoxy, alkenyloxy, alkynyloxy, C₃-C₆-cycloalkyl, C₃-C₆-halogencycloalkyl, C₁-C₄-haloalkoxy, C₁-C₆-alkylthio, C(═O) and C(═S); or
R⁹ & R¹⁰ together with the carbon atoms to which they are bound form a five-, six-, or seven-membered carbo- and heterocyclic or heteroaromatic ring; wherein the heterocyclic or heteroaromatic ring contains 1, 2, 3 or 4 heteroatoms selected from the group consisting of N, O and S, wherein N may carry one substituent R^{N} selected from the group consisting of C₁-C₄-alkyl, C₁-C₄-haloalkyl and SO₂Ph, wherein Ph is unsubstituted or substituted by substituents selected from C₁-C₄-alkyl, and wherein S may be in the form of its oxide SO or SO_{2;} and wherein in each case one or two CH₂ groups of the carbo- or heterocycle may be replaced by a group independently selected from the group consisting of C(═O) and C(═S); and wherein the carbo- and heterocyclic or heteroaromatic ring is substituent by (R^{H}), wherein m is 0, 1, 2, 3 or 4;
R¹¹ is in each case independently selected from the group consisting of halogen, OH, CN, NO₂, SH, NH₂,NH(C₁-C₄-alkyl), N(C₁-C₄-alkyl)₂, NH—SO₂—R^{x}, C₁-C₆-alkyl, C₂-C₆-alkenyl, C₂-C₆-alkynyl, C₁-C₆-alkoxy, C₃-C₆-cycloalkyl, five- or six-membered heteroaryl and aryl; wherein the heteroaryl contains 1, 2 or 3 heteroatoms selected from the group consisting of N, O and S; and wherein in each case one or two CH₂ groups of the carbo- or heterocycle may be replaced by a group independently selected from the group consisting of C(═O) and C(═S); and wherein
R^{x} is selected from the group consisting of C₁-C₄-alkyl, C₁-C₄-haloalkyl, aryl which is unsubstituted or substituted by substituents R^{d} independently selected from C₁-C₄-alkyl;
wherein the acyclic moieties of R¹¹ are unsubstituted or substituted with identical or different groups R^{11a} which independently of one another are selected from the group consisting of:
R^{11a} is selected from the group consisting of halogen, OH, CN, C₁-C₆-alkoxy, C₃-C₆-cycloalkyl, C₃-C₆-halocycloalkyl, C₁-C₄-haloalkoxy, C₁-C₆-alkylthio and phenoxy, wherein the phenyl group is unsubstituted or unsubstituted or substituted with R^{11a} selected from the group consisting of halogen, OH, C₁-C₄-alkyl, C₁-C₄-haloalkyl, C₁-C₄-alkoxy and C₁-C₄-haloalkoxy; wherein the cycloalkyl, heteroaryl and aryl moieties of R^{H} unsubstituted or substituted with identical or different groups R^{11b} which independently of one another are selected from the group consisting of:
R^{11b} is selected from the group consisting of halogen, OH, CN, C₁-C₄-alkyl, C₁-C₄-alkoxy, C₁-C₄-haloalkyl, C₃-C₆-cycloalkyl, C₃-C₆-halocycloalkyl, C₁-C₄-haloalkoxy, and C₁-C₆-alkylthio;
wherein if ring A is phenyl, Y is not F;
or an N-oxide or a agriculturally acceptable salt thereof.

17. The compound of claim 16, wherein ring A-W—Y is selected from below group: 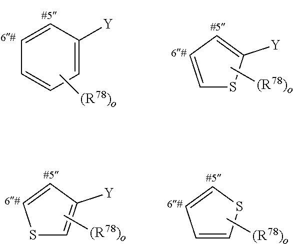
wherein, the positions of the rings marked with “#” represents the connection points (carbon atoms 5″ and 6″ in formula I) with the remaining skeleton of the compounds of formula I.

18. The compound of claim 16, wherein R³, and R⁴ are independently selected from the group consisting of C₁-C₄-alkyl and C₁-C₄-haloalkyl.

19. The compound of claim 16, wherein R³ is selected from the group consisting of CH₃, CH₂F, CHF₂ and CF₃.

20. The compound of claim 16, wherein R⁴ is selected from the group consisting of CN, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₁-C₆-alkoxy, C₁-C₆-haloalkoxy, CH(═O), C(═O)C₁-C₆-alkyl, C(═O)OC₁-C₆-alkyl, CR^{x}═NR^{x}, a saturated three-, four-, five-, six-, membered carbocycle or heterocycle, a five- or six-membered heteroaryl or aryl; and C₁-C₆-alkyl substituted by CN, C₁-C₆-alkoxy, C₁-C₄-haloalkoxy, C₁-C₆-alkylthio, N(C₁-C₆-alkyl)₂, CH(═O), C(═O)C₁-C₆-alkyl, C(═O)OC₁-C₆-alkyl, CR^{x}═NR^{x}, a saturated three-, four-, five-, six-, membered carbocycle or heterocycle, and aryl.

21. The compound of claim 16, wherein R⁹ and R¹⁰ independently are selected from the group consisting of C₁-C₄-alkyl, H, CN, halogen, C₁-C₆-haloalkyl, C₂-C₆-alkynyl, O(R⁹⁵), and C₃-C₆-cycloalkyl, wherein
R^{Y} is selected from the group consisting of C₁-C₆-alkyl, and C₁-C₆-haloalkyl.

22. The compound of claim 16, wherein R¹ and R² independently are selected from the group consisting of hydrogen, halogen, C₁-C₄-alkyl and C₁-C₄-alkenyl.

23. The compound of claim 16, wherein Y is independently of one another selected from the group consisting of halogen, C₁-C₄-alkoxy, C₁-C₄-haloalkoxy, C₁-C₄-alkyl and C₁-C₄-haloalkyl.

24. A process for the synthesis of compound I of claim 16, comprising the step of:
a) reacting a compound X

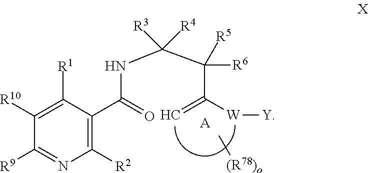

25. The intermediate compound of formula X: 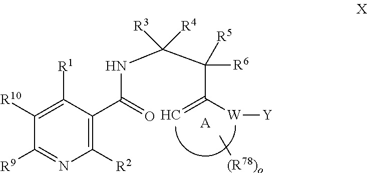
wherein the substituents are defined as in claim 16 and wherein if R¹ is H, both R⁹ and R¹⁰ cannot be H.

26. A composition comprising the compound of formula I, as defined in claim 16, an N-oxide or an agriculturally acceptable salt thereof.

27. The composition of claim 26, additionally comprising a further active substance.

28. A method for combating phytopathogenic fungi comprising treating the fungi or the materials, plants, the soil or seeds to be protected against fungal attack with an effective amount of at least one compound of formula I, as defined in claim 16.

29. A seed coated with at least one compound of formula I, as defined in claim 16, and/or an agriculturally acceptable salt thereof, in an amount of from 0.1 to 10 kg per 100 kg of seed.

30. A method for combating phytopathogenic fungi comprising treating the fungi or the materials, plants, the soil or seeds to be protected against fungal attach with an effective amount of the composition of claim 26.

31. A seed coated with the composition of claim 26 in an amount of from 0.1 to 10 kg per 100 kg of seed.
